(19) United States
(12) Patent Application Publication
(10) Pub. No.: US 2022/0290164 A1
(43) Pub. Date: Sep. 15, 2022

(54) RECOMBINANT RABIES VIRUSES FOR GENE THERAPY

(71) Applicant: BEAM THERAPEUTICS INC,, Cambridge, MA (US)

(72) Inventors: Fei Ran, Cambridge, MA (US); ChieYu Lin, Cambridge, MA (US)

(21) Appl. No.: 17/651,738

(22) Filed: Feb. 18, 2022

Related U.S. Application Data
(60) Provisional application No. 63/241,989, filed on Sep. 8, 2021.
(60) Provisional application No. 63/151,542, filed on Feb. 19, 2021.

(51) Int. Cl. C12N 15/66; C12N 9/78; C12N 9/22; C12N 15/90; A61K 31/7088

(52) U.S. Cl. CPC C12N 15/66; C12N 9/78; C12N 9/22; C12Y 305/04004; C12Y 305/04005; C12N 15/90; A61K 31/7088; C12N 2310/20; C07K 2319/81; C07K 2319/09

(57) ABSTRACT

Provided herein are recombinant rabies virus genomes and recombinant rabies viruses and methods for their use in delivering a transgene into a target cell. Also provided are packaging systems and methods of using the packaging systems to produce recombinant rabies viruses.

### RELATED APPLICATIONS

This application claims the benefit of U.S. Provisional Patent Application Ser. No. 63/151,542, filed Feb. 19, 2021, and U.S. Provisional Patent Application Ser. No. 63/241,989, filed Sep. 8, 2021, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII copy, created on May 31, 2022, is named 725423_BEAM9-002_SL.txt and is 1,908,736 bytes in size.

### BACKGROUND

Gene therapies largely involve the use of viral gene delivery systems in order to transduce a cell of interest and express a transgene. Viral systems that are commonly used for gene therapy are derived from viruses and suffer from significant disadvantages. The challenges in using current viral systems include: limited, small packaging capacities; unintended resultant consequences such as genomic integration; limited tissue tropism; pre-existing immunity and/or immune responses in the target population, limited ability for re-dosing; and limited durability due to genome instability, immune clearance, and cellular toxicity. For example, while adenoviral vector-mediated gene therapy demonstrates high transduction efficiency and can be used to infect many different cell types, certain disadvantages include non-integration and high immunogenicity. Disadvantages of adeno-associated viral vector-mediated gene therapy include high immunogenicity, and limited packaging capacity. As another example, retroviral vector-mediated gene therapy suffers from low transduction efficiency and the inactivation by complement.

Accordingly, there is a need for novel viral gene delivery systems that are advantages over current viral systems.

### SUMMARY

Provided herein are recombinant viral vectors and recombinant viruses derived from the rabies virus, which can be used to transduce a target cell and express a transgene therein. The recombinant rabies vectors and viruses provided by the present disclosure find use as effective viral gene delivery systems. Also provided are viral packaging systems and methods of producing the recombinant viruses described herein.

In one aspect, a recombinant rabies virus genome, comprising a nucleic acid encoding a therapeutic transgene, wherein: the genome lacks a G gene encoding for a rabies virus glycoprotein or a functional variant thereof; and/or the genome lacks an L gene encoding for a rabies virus polymerase or a functional variant thereof, is provided.

In certain exemplary embodiments, the genome lacks a G gene encoding for a rabies virus glycoprotein or a functional variant thereof. In certain exemplary embodiments, the genome lacks a G gene encoding for a rabies virus glycoprotein or a functional variant thereof, and wherein the genome lacks an L gene encoding for a rabies virus polymerase or a functional variant thereof.

In certain exemplary embodiments, the genome comprises: an N gene encoding for a rabies virus nucleoprotein or a functional variant thereof; a P gene encoding for a rabies virus phosphoprotein or a functional variant thereof; and an M gene encoding for a rabies virus matrix protein or a functional variant thereof.

In another aspect, a recombinant rabies virus particle, comprising a rabies virus glycoprotein and a recombinant rabies virus genome as described herein, is provided.

In another aspect, a recombinant rabies virus particle, comprising: a rabies virus glycoprotein; and a recombinant rabies virus genome comprising a nucleic acid encoding a therapeutic transgene, wherein: the genome lacks a G gene encoding for a rabies virus glycoprotein or a functional variant thereof; and/or the genome lacks an L gene encoding for a rabies virus polymerase or a functional variant thereof, is provided.

In certain exemplary embodiments, the genome lacks a G gene encoding for a rabies virus glycoprotein or a functional variant thereof. In certain exemplary embodiments, the genome lacks a G gene encoding for a rabies virus glycoprotein or a functional variant thereof, and wherein the genome lacks an L gene encoding for a rabies virus polymerase or a functional variant thereof.

In certain exemplary embodiments, the genome lacks:

a G gene encoding for a rabies virus glycoprotein or a functional variant thereof;

an L gene encoding for a rabies virus polymerase or a functional variant thereof; and

an M gene encoding for a rabies virus matrix protein or a functional variant thereof.

In certain exemplary embodiments, the genome lacks:

a G gene encoding for a rabies virus glycoprotein or a functional variant thereof;

an L gene encoding for a rabies virus polymerase or a functional variant thereof;

an M gene encoding for a rabies virus matrix protein or a functional variant thereof; and

a P gene encoding for a rabies virus phosphoprotein or a functional variant thereof.

In certain exemplary embodiments, the genome lacks:

a G gene encoding for a rabies virus glycoprotein or a functional variant thereof;

an L gene encoding for a rabies virus polymerase or a functional variant thereof;

an M gene encoding for a rabies virus matrix protein or a functional variant thereof;

a P gene encoding for a rabies virus phosphoprotein or a functional variant thereof; and

an N gene encoding for a rabies virus nucleoprotein or a functional variant thereof.

In certain exemplary embodiments, the genome only encodes a therapeutic transgene.

In certain exemplary embodiments, the genome comprises: an N gene encoding for a rabies virus nucleoprotein or a functional variant thereof; a P gene encoding for a rabies virus phosphoprotein or a functional variant thereof; and an M gene encoding for a rabies virus matrix protein or a functional variant thereof.

In other exemplary embodiments, the genome lacks: an N gene encoding for a rabies virus nucleoprotein or a functional variant thereof; a P gene encoding for a rabies virus phosphoprotein or a functional variant thereof; and/or an M gene encoding for a rabies virus matrix protein or a functional variant thereof.

In certain exemplary embodiments, the recombinant rabies virus is replication incompetent. In other exemplary embodiments, the recombinant rabies virus is replication deficient.

In certain exemplary embodiments, each of the genes are operably linked to a transcriptional regulatory element. In certain exemplary embodiments, the transcriptional regulatory element comprises a transcription initiation signal. In certain exemplary embodiments, the transcription initiation signal is exogenous to the rabies virus. In certain exemplary embodiments, the transcription initiation signal is endogenous to the rabies virus.

In certain exemplary embodiments, each of the genes are operably linked to a transcription termination polyadenylation signal.

In certain exemplary embodiments, the therapeutic transgene comprises a sequence that encodes a nucleic acid editing system or a component thereof. In certain exemplary embodiments, the nucleic acid editing system comprises a Clustered Regulatory Interspaced Short Palindromic Repeat (CRISPR) system, a zinc finger nuclease (ZFN), a meganuclease, and a Transcription Activator-Like Effector-based Nucleases (TALEN). In certain exemplary embodiments, the nucleic acid editing system comprises a CRISPR system. In certain exemplary embodiments, the CRISPR-system comprises a nucleobase editor comprising a polynucleotide programmable nucleotide binding domain and a nucleobase editing domain.

In certain exemplary embodiments, the nucleobase editing domain is an adenosine deaminase, cytidine deaminase, or a functional variant thereof. In certain exemplary embodiments, the nucleobase editing domain is an adenosine deaminase. In certain exemplary embodiments the adenosine deaminase comprises a TadA deaminase from any of the adenosine base editors recited in Table 10, Table 11, Table 12, Table 13, Table 14, or Table 15.

In certain exemplary embodiments the nucleobase editing domain comprises a adenosine deaminase from any one of the adenosine base editors of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58.

In certain exemplary embodiments the nucleobase editing domain comprises a adenosine base editor selected from the group consisting of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58. In certain exemplary embodiments, the adenosine deaminase is ABE7.10 or ABE8.20.

In certain exemplary embodiments the nucleobase editing domain comprises a cytidine deaminase. In certain exemplary embodiments the cytidine deaminase is selected from the group consisting of: Petromyzon marinus cytosine deaminase 1 (PmCDA1), Activation-induced cytidine deaminase (AID), and APOBEC.

In certain exemplary embodiments, the polynucleotide programmable nucleotide binding domain comprises a Cas9 polypeptide, a Cas12 polypeptide, or a functional variant thereof.

In certain exemplary embodiments, the CRISPR-system further comprises a guide RNA (gRNA) or a nucleic acid sequence encoding a gRNA.

In certain exemplary embodiments, the therapeutic transgene comprises a sequence encoding a uracil glycosylase inhibitor (UGI).

In certain exemplary embodiments, the therapeutic transgene comprises a sequence encoding a nuclear localization signal (NLS).

In certain exemplary embodiments, the polynucleotide programmable nucleotide binding domain and/or the nucleobase editing domain further comprises a nuclear localization signal (NLS).

In certain exemplary embodiments, the cytidine deaminase further comprises a uracil glycosylase inhibitor (UGI).

In certain exemplary embodiments, the therapeutic transgene comprises a therapeutic polypeptide and/or a therapeutic nucleic acid. In certain exemplary embodiments, the therapeutic polypeptide and/or therapeutic nucleic acid is secreted from a cell.

In certain exemplary embodiments, the nucleic acid encoding the therapeutic transgene is greater than: about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, about 2,000 bp, about 2,100 bp, about 2,200 bp, about 2,300 bp, about 2,400 bp, about 2,500 bp, about 2,600 bp, about 2,700 bp, about 2,800 bp, about 2,900 bp, or about 3,000 bp. In certain exemplary embodiments, the nucleic acid encoding the therapeutic transgene is greater than about 300 bp. In certain exemplary embodiments, the nucleic acid encoding the therapeutic transgene is greater than about 650 bp. In certain exemplary embodiments, the nucleic acid encoding the therapeutic transgene is greater than about 1,000 bp. In certain exemplary embodiments, the nucleic acid encoding the therapeutic transgene is greater than about 3,000 bp. In certain exemplary embodiments, the nucleic acid encoding the therapeutic transgene is greater than about 4,500 bp. In certain exemplary embodiments, the nucleic acid encoding the therapeutic transgene is greater than about 8,500 bp. In certain exemplary embodiments, the nucleic acid encoding the therapeutic transgene is greater than about 10,000 bp.

In certain exemplary embodiments, the therapeutic transgene is operably linked to a transcriptional regulatory element. In certain exemplary embodiments, the transcriptional regulatory element comprises a transcription initiation signal. In certain exemplary embodiments, the transcription initiation signal is exogenous to the rabies virus. In certain exemplary embodiments, the transcription initiation signal is endogenous to the rabies virus.

In certain exemplary embodiments, the therapeutic transgene is operably linked to a transcription termination polyadenylation signal.

In another aspect, a pharmaceutical composition comprising a recombinant virus particle as described herein, is provided.

In another aspect, a method for expressing a therapeutic transgene in a target cell, comprising transducing a target cell with a recombinant virus particle as described herein, is provided.

In another aspect, a method for expressing a nucleobase editor in a target cell, comprising transducing a target cell with a recombinant rabies virus particle, wherein the recombinant virus particle comprises: a rabies virus glycoprotein; and a recombinant rabies virus genome comprising a nucleic acid encoding a nucleobase editor comprising a polynucleotide programmable nucleotide binding domain and a nucleobase editing domain, wherein: the genome lacks a G gene encoding for a rabies virus glycoprotein or a functional variant thereof; and/or the genome lacks an L gene encoding for a rabies virus polymerase or a functional variant thereof, is provided.

In certain exemplary embodiments, the genome further lacks:

an M gene encoding for a rabies virus matrix protein or a functional variant thereof.

In certain exemplary embodiments, the genome further lacks:

an M gene encoding for a rabies virus matrix protein or a functional variant thereof; and

a P gene encoding for a rabies virus phosphoprotein or a functional variant thereof.

In certain exemplary embodiments, the genome further lacks:

an M gene encoding for a rabies virus matrix protein or a functional variant thereof;

a P gene encoding for a rabies virus phosphoprotein or a functional variant thereof; and

an N gene encoding for a rabies virus nucleoprotein or a functional variant thereof.

In certain exemplary embodiments, the genome only encodes a nucleobase editor.

In certain exemplary embodiments, the genome comprises: an N gene encoding for a rabies virus nucleoprotein or a functional variant thereof; a P gene encoding for a rabies virus phosphoprotein or a functional variant thereof; and an M gene encoding for a rabies virus matrix protein or a functional variant thereof.

In certain exemplary embodiments, each of the genes are operably linked to a transcriptional regulatory element. In certain exemplary embodiments, the transcriptional regulatory element comprises a transcription initiation signal. In certain exemplary embodiments, the transcription initiation signal is exogenous to the rabies virus. In certain exemplary embodiments, the transcription initiation signal is endogenous to the rabies virus.

In certain exemplary embodiments, each of the genes are operably linked to a transcription termination polyadenylation signal.

In certain exemplary embodiments, the nucleobase editing domain comprises an adenosine deaminase, cytidine deaminase, or a functional variant thereof.

In certain exemplary embodiments, the nucleobase editing domain comprises an adenosine deaminase. In certain exemplary embodiments, the adenosine deaminase comprises a TadA deaminase from any of the adenosine base editors recited in Table 10, Table 11, Table 12, Table 13, Table 14, or Table 15.

In certain exemplary embodiments, the nucleobase editing domain comprises a adenosine deaminase from any one of the adenosine base editors of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58.

In certain exemplary embodiments, the nucleobase editing domain comprises a adenosine base editor selected from the group consisting of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58.

In certain exemplary embodiments, the nucleobase editing domain comprises a adenosine base editor of ABE7.10 or ABE8.20.

In certain exemplary embodiments, the nucleobase editing domain is a cytidine deaminase. In certain exemplary embodiments, the cytidine deaminase is selected from the group consisting of: Petromyzon marinus cytosine deaminase 1 (PmCDA1), Activation-induced cytidine deaminase (AID), and APOBEC.

In certain exemplary embodiments, the polynucleotide programmable nucleotide binding domain comprises a Cas9 polypeptide, a Cas12 polypeptide, or a functional variant thereof.

In certain exemplary embodiments, the recombinant genome further comprises a guide RNA (gRNA). In certain exemplary embodiments, the guide RNA (gRNA) is provided to the target cell exogenously. In certain exemplary embodiments, the gRNA is capable of targeting a genomic locus of the target cell.

In certain exemplary embodiments, the target cell is transduced ex vivo. In certain exemplary embodiments, the target cell is a human cell. In certain exemplary embodiments, the target cell is obtained from a human. In certain exemplary embodiments, the target cell is autologous to the human. In certain exemplary embodiments, the target cell is allogeneic to the human.

In certain exemplary embodiments, the target cell is transduced in vivo. In certain exemplary embodiments, the target cell is a human cell. In certain exemplary embodiments, the target cell is a neuronal cell, an epithelial cell, or a hepatocyte. In certain exemplary embodiments, the target cell is in a human.

In another aspect, a method for delivering a therapeutic transgene to a subject, comprising administering to the subject a recombinant virus particle as described herein, or a pharmaceutical composition as described herein, is provided.

In another aspect, a method for delivering a nucleobase editor to a subject, comprising administering to the subject a recombinant rabies virus particle, wherein the recombinant virus particle comprises: a rabies virus glycoprotein; and a recombinant rabies virus genome comprising a nucleic acid encoding a nucleobase editor comprising a polynucleotide programmable nucleotide binding domain and a nucleobase editing domain, wherein: the genome lacks a G gene encoding for a rabies virus glycoprotein or a functional variant thereof;

and/or the genome lacks an L gene encoding for a rabies virus polymerase or a functional variant thereof, is provided.

In certain exemplary embodiments, wherein the genome comprises: an N gene encoding for a rabies virus nucleoprotein or a functional variant thereof; a P gene encoding for a rabies virus phosphoprotein or a functional variant thereof; and an M gene encoding for a rabies virus matrix protein or a functional variant thereof.

In certain exemplary embodiments, wherein the nucleobase editing domain is an adenosine deaminase, cytidine deaminase, or a functional variant thereof. In certain exemplary embodiments, the base editor is an adenosine deaminase. In certain exemplary embodiments, the adenosine deaminase is ABE7.10.

In certain exemplary embodiments, the polynucleotide programmable nucleotide binding domain comprises a Cas9 polypeptide, a Cas12 polypeptide, or a functional variant thereof.

In certain exemplary embodiments, the recombinant genome further comprises a nucleic acid sequence encoding a guide RNA (gRNA). In certain exemplary embodiments, the guide RNA (gRNA) is provided to the target cell exogenously. In certain exemplary embodiments, the gRNA is capable of targeting a genomic locus of the target cell.

In certain exemplary embodiments, the subject is a mammal. In certain exemplary embodiments, the subject is a human.

In another aspect, a packaging system for the recombinant preparation of a rabies virus particle, wherein the packaging system comprises: an N gene encoding for a rabies virus nucleoprotein or a functional variant thereof; a P gene encoding for a rabies virus phosphoprotein or a functional variant thereof; an L gene encoding for a rabies virus polymerase or a functional variant thereof; and a recombinant rabies virus genome, wherein: the genome lacks a G gene encoding for a rabies virus glycoprotein or a functional variant thereof; and/or the genome lacks an L gene encoding for a rabies virus polymerase or a functional variant thereof, is provided.

In certain exemplary embodiments, the recombinant rabies virus genome comprises a nucleic acid encoding a transgene or therapeutic transgene.

In certain exemplary embodiments, the recombinant rabies virus genome is comprised within a virus genome vector.

In certain exemplary embodiments, the N, P, and L genes are each comprised within a separate vector. In certain exemplary embodiments, each of the N, P, and L genes are operably linked to a transcriptional regulatory element. In certain exemplary embodiments, the transcriptional regulatory element comprises a promoter and/or enhancer. In certain exemplary embodiments, the promoter is a constitutive promoter. In certain exemplary embodiments, the promoter is an elongation factor 1a promoter. In certain exemplary embodiments, the separate vectors are each contained within a separate transfecting plasmid.

In certain exemplary embodiments, the N, P, and L genes are comprised within a single vector. In certain exemplary embodiments, the single vector comprises a first expression cassette comprising the N and P genes, and a second expression cassette comprising the L gene. In certain exemplary embodiments, the first expression cassette comprises from 5′ to 3′: a transcriptional regulatory element; the P gene; and the N gene. In certain exemplary embodiments, the first expression cassette comprises from 5′ to 3′: a transcriptional regulatory element; the P gene; a ribosomal skipping element; and the N gene. In certain exemplary embodiments, the ribosomal skipping element is an IRES element. In certain exemplary embodiments, the ribosomal skipping element is a 2A element. In certain exemplary embodiments, the second expression cassette comprises from 5′ to 3′: a transcriptional regulatory element; and the L gene. In certain exemplary embodiments, the transcriptional regulatory element comprises a promoter and/or enhancer. In certain exemplary embodiments, the promoter is a constitutive promoter. In certain exemplary embodiments, the promoter is an elongation factor 1α promoter. In certain exemplary embodiments, the first and the second expression cassettes are in opposite orientations in the vector. In certain exemplary embodiments, the single vector is contained within a single transfecting plasmid.

In certain exemplary embodiments, the packaging system further comprises an M gene encoding for a rabies virus matrix protein or a functional variant thereof. In certain exemplary embodiments, the M gene is comprised within a vector. In certain exemplary embodiments, the M gene is operably linked to a transcriptional regulatory element. In certain exemplary embodiments, the transcriptional regulatory element comprises a promoter and/or enhancer. In certain exemplary embodiments, the vector comprising the M gene is contained within a transfecting plasmid.

In certain exemplary embodiments, the packaging system further comprises a G gene encoding for a rabies virus glycoprotein or a functional variant thereof. In certain exemplary embodiments, the G gene is comprised within a vector. In certain exemplary embodiments, the G gene is operably linked to a transcriptional regulatory element. In certain exemplary embodiments, the transcriptional regulatory element comprises a promoter and/or enhancer.

In certain exemplary embodiments, the vector comprising the G gene is contained within a transfecting plasmid.

In another aspect, a method for producing a recombinant rabies virus particle, the method comprising introducing a packaging system as described herein into a cell under conditions operative for enveloping the recombinant rabies virus genome to form the recombinant rabies virus particle, is provided.

In certain exemplary embodiments, the introducing is mediated by electroporation, nucleofection, or lipofection.

In another aspect, a recombinant rabies virus particle packaging cell comprising a packaging system as described herein, is provided.

In another aspect, a recombinant rabies virus particle packaging cell, comprising: an N gene encoding for a rabies virus nucleoprotein or a functional variant thereof; a P gene encoding for a rabies virus phosphoprotein or a functional variant thereof; and an L gene encoding for a rabies virus polymerase or a functional variant thereof, is provided.

In certain exemplary embodiments, a first expression cassette comprises the N and P genes. In certain exemplary embodiments, the first expression cassette comprises from 5′ to 3′: a transcriptional regulatory element; the P gene; and the N gene. In certain exemplary embodiments, the first expression cassette comprises from 5′ to 3′: a transcriptional regulatory element; the P gene; a ribosomal skipping element; and the N gene. In certain exemplary embodiments, the ribosomal skipping element is an IRES element. In certain exemplary embodiments, the ribosomal skipping element is a 2A element.

In certain exemplary embodiments, a second expression cassette comprises the L gene. In certain exemplary embodiments, the second expression cassette comprises from 5′ to 3′: a transcriptional regulatory element; and the L gene. In certain exemplary embodiments, the transcriptional regulatory element comprises a promoter and/or enhancer. In certain exemplary embodiments, the promoter is a constitutive promoter. In certain exemplary embodiments, the promoter is an elongation factor 1α promoter.

In certain exemplary embodiments, the first and the second expression cassettes are in opposite orientations in the vector.

In certain exemplary embodiments, the recombinant rabies virus particle packaging cell further comprises an M gene encoding for a rabies virus matrix protein and/or a G gene encoding for a rabies virus glycoprotein. In certain exemplary embodiments, the M gene and/or the G gene are operably linked to a transcriptional regulatory element. In certain exemplary embodiments, the transcriptional regulatory element comprises a promoter and/or enhancer. In certain exemplary embodiments, a third expression and/or fourth cassette comprises the rabies virus G gene and/or rabies virus M gene.

In certain exemplary embodiments, the packaging cell is of a mammalian, a bacterial, or an insect origin. In certain exemplary embodiments, the packaging cell is selected from the group consisting of a HEK293 cell, a VERO cell, a BHK cell, and a BSR cell.

In another aspect, a method for producing a recombinant rabies virus, the method comprising introducing into a packaging cell as described herein, a nucleic acid comprising a recombinant rabies virus genome comprising a nucleic acid comprising a therapeutic transgene, under conditions operative for enclosing the recombinant rabies virus genome in the glycoprotein to form the recombinant rabies virus, wherein: the genome lacks an endogenous G gene encoding for a rabies virus glycoprotein; and the genome lacks an endogenous L gene encoding for a rabies virus polymerase, is provided.

In certain exemplary embodiments, the recombinant genome comprises: an N gene encoding for a rabies virus nucleoprotein or a functional variant thereof; a P gene encoding for a rabies virus phosphoprotein or a functional variant thereof; and an M gene encoding for a rabies virus matrix protein or a functional variant thereof.

In certain exemplary embodiments, each of the genes are operably linked to a transcriptional regulatory element. In certain exemplary embodiments, the transcriptional regulatory element comprises a transcription initiation signal. In certain exemplary embodiments, the transcription initiation signal is exogenous to the rabies virus. In certain exemplary embodiments, the transcription initiation signal is endogenous to the rabies virus.

In certain exemplary embodiments, each of the genes are operably linked to a transcription termination polyadenylation signal.

In certain exemplary embodiments, the recombinant rabies virus titer is greater than about 1E8 TU/mL. In certain exemplary embodiments, the recombinant rabies virus titer is from about 1E8 TU/mL to about 1E9 TU/mL.

In another aspect, a method of treating a disease or disorder in a subject, the method comprising administering a recombinant rabies virus particle as described herein, or a pharmaceutical composition as described herein to the subject, is provided.

In certain exemplary embodiments, the disease or disorder is a neurologic disease or disorder. In certain exemplary embodiments, the disease or disorder is an ophthalmic disease or disorder.

In another aspect, use of a recombinant rabies virus as described herein, or a pharmaceutical composition as described herein, in the manufacture of a medicament for treating a disease or disorder in a subject, is provided.

### BRIEF DESCRIPTION OF THE DRAWINGS

FIG. 1 is a chart showing relative infectivity on 293T cells from equal volumes of virus-containing supernatant harvested on the indicated days from various stable cell lines.

FIG. 2A is a schematic depicting the VIR218 replicon.

FIG. 2B is a schematic depicting the production and infection scheme for recombinant rabies virus particle mediated gene delivery.

FIG. 2C is a chart depicting that a recombinant rabies virus particle comprising a recombinant rabies virus genome encoding a nucleobase editor can effect gene editing of a target sequence.

FIG. 3A is a schematic depicting the organization of a recombinant rabies viral genome comprising a gRNA, polynucleotide programmable nucleotide binding domain, and nucleobase editors.

FIG. 3B is a schematic depicting a gRNA-tRNA expression cassette encoding a gRNA between two tRNA sequences with arrows indicating cleavage sites of the RNA.

FIG. 3C is a schematic depicting a gRNA-tRNA expression cassette encoding gRNAs (a first gRNA and a second gRNA), wherein the first gRNA is between a first tRNA and a second tRNA, followed by the second gRNA.

FIG. 3D is a schematic depicting a gRNA-tRNA expression cassette encoding gRNAs (a first gRNA and a second gRNA), wherein the first gRNA is between a first tRNA and a second tRNA, and the second gRNA is between a second tRNA and a third tRNA.

FIG. 3E is a chart depicting % infection and % A>G base editing in HEK cells transduced with a recombinant rabies virus particle comprising a recombinant rabies virus genome encoding a nucleobase editor and gRNAs encoded between multiple tRNAs. The base editing was measured at a Hek2 site and IEDG site targeted by a Hek2-targeting gRNA and a IEDG-targeting gRNA.

FIG. 4A is a schematic depicting a ΔG, ΔGL, and ΔALL (i.e., ΔGLNPM) rabies virus replicon encoding a Cre recombinase-2A-GFP reporter gene.

FIG. 4B depicts flourescent images of cells transfected with rabies virus vectors in a ΔG, ΔGL, or ΔALL (i.e., ΔGLNPM) background, each vector encoding a Cre recombinase-2A-GFP reporter gene. Images were taken 6 days post-transfection under a standard transfection and an optimized transfection as depicted in FIG. 2B.

FIG. 4C depicts bar graphs representing % viral entry into reporter cells, as measured by GFP flourescence.

FIG. 5 is a chart depicting percent A to G base editing in 293T cells tranduced with RABV having a RABV genome encoding the ABE 8.20 base editor. A codon-optimized version of the RABV SAD B19 ΔG virus, referred to as SynV, was used as the RABV genome. The base editor gene replaced the G gene in the RABV genome. U6-driven guide RNAs (gRNAs), targeting HEK2 and ABCA4 loci, were transfected in trans into target cells at the time of infection. Multiplicity of infection (MOI) based on functional viral titers is indicated.

FIG. 6 depicts two charts depicting percent C to T base editing in 293T cells tranduced with RABV having a RABV genome encoding one of several different base editors. The SynV RABV genome was used. The base editor gene replaced the G gene in the RABV genome. U6-driven gRNAs, targeting HEK2 (top chart) and ABCA4 (bottom chart) loci, were transfected in trans into target cells at the time of infection. MOI based on functional viral titers is indicated.

FIG. 7 depicts two charts depicting percent A to G base editing in 293T cells tranduced with RABV having a RABV genome encoding the ABE7.10 base. A SAD B19 ΔGL RABV genome was used to encode the base editor, where the base editor replaced the G protein within the RABV genome. One set of cells was additionally transfected by a plasmid encoding RABV L protein (virus+L). U6-driven gRNAs, targeting HEK2 and ABCA4 loci, were transfected in trans into target cells at the time of infection. MOI based on functional viral titers is indicated.

FIG. 8 depicts flourescent images of cells transfected with rabies virus vectors in a ΔN, ΔP, ΔM, and ΔL background, each vector encoding a Cre recombinase-2A-GFP reporter gene. Either 293T cells or CE1.30 cells were transfected, with and without complementation of the missing gene. Images were taken 4 days post-insfection.

FIG. 9 depicts bar graphs representing % viral entry into reporter cells (293T or CE1.30), as measured by GFP flourescence. Supernantant from the cells transfected in FIG. 8 were used.

FIG. 10 depicts graphs representing % viral entry into CE1.30 cells, as measured by GFP and mScarlet flourescence. ΔGL, ΔMGL, ΔPMGL, and ΔALL rabies virus replicons encoding Cre-2a-GFP as a transgene were grown in CE1.30 cells, and titered on reporter cells to show functional delivery of both Cre and GFP mRNA.

FIG. 11 depicts flourescent images of cells transfected with rabies virus vectors in a ΔGL, ΔMGL, ΔPMGL, and ΔALL background, each vector encoding a Cre recombinase-2A-GFP reporter gene. Either 293T cells or CE1.30 cells were transfected. Images were taken 3 days post-insfection.

FIG. 12 depicts schmeatics of expression vectors used to express RABV genes. Vector VIR069 contains a first expression cassette, from 5′ to 3′, of an EF1α promoter, the RABV N gene, a 2a ribosomal skipping element, the RABV P gene, a 2a ribosomal skipping element, and the RABV M gene. VIR069 contains a second expression cassette in the reverse orientation to the first expression cassette, from 5′ to 3′, of an RPBSA promoter and a BFP-zeocin selection marker gene. Vector VIR071 contains a first expression cassette, from 5′ to 3′, of an EF1α promoter, the RABV N gene, an IRES, the RABV P gene, a 2a ribosomal skipping element, and the RABV M gene. VIR071 contains a second expression cassette in the reverse orientation to the first expression cassette, from 5′ to 3′, of an RPBSA promoter, the RABV L gene, a 2a ribosomal skipping element, and a zeocin selection marker gene. Vector VIR112 contains a first expression cassette, from 5′ to 3′, of an EF1α promoter, the RABV N gene, an IRES, the RABV P gene, an IRES, and the RABV M gene. VIR071 contains a second expression cassette in the reverse orientation to the first expression cassette, from 5′ to 3′, of an RPBSA promoter, the RABV L gene, a 2a ribosomal skipping element, and a zeocin selection marker gene.

### DETAILED DESCRIPTION

Provided herein is a recombinant rabies virus genome that comprises a nucleic acid comprising a transgene (e.g., a therapeutic transgene). In certain embodiments, the recombinant rabies virus genome lacks an endogenous G gene encoding for a rabies virus glycoprotein, and lacks an endogenous L gene encoding for a rabies virus polymerase. Also provided are methods for producing the recombinant rabies virus particles. Such methods generally comprise introducing a packaging system described herein into a suitable packaging cell.

It is to be understood that the methods described herein are not limited to particular methods and experimental conditions disclosed herein as such methods and conditions may vary. It is also to be understood that the terminology used herein is for the purpose of describing particular embodiments only, and is not intended to be limiting. The methods described herein use conventional molecular and cellular biological and immunological techniques that are well within the skill of the ordinary artisan. Such techniques are well known to the skilled artisan and are explained in the scientific literature.

### A. DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “adenosine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g. engineered adenosine deaminases, evolved adenosine deaminases) provided herein may be from any organism, such as a bacterium.

By “Adenosine Deaminase Base Editor 8 (ABE8) polypeptide” or “ABE8” is meant a base editor as defined herein comprising an adenosine deaminase variant comprising an alteration at amino acid position 82 and/or 166 of the following reference sequence:

(SEQ ID NO: 8)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD.

In some embodiments, ABE8 comprises further alterations, as described herein, relative to the reference sequence.

By “Adenosine Deaminase Base Editor 8 (ABE8) polynucleotide” is meant a polynucleotide encoding an ABE8.

“Administering” is referred to herein as providing one or more compositions described herein to a patient or a subject.

By “agent” is meant any small molecule chemical compound, antibody, nucleic acid molecule, or polypeptide, or fragments thereof.

By “alteration” is meant a change (increase or decrease) in the level, structure, or activity of an analyte, gene or polypeptide as detected by standard art known methods such as those described herein. As used herein, an alteration includes a 10% change in expression levels, a 25% change, a 40% change, and a 50% or greater change in expression levels. In some embodiments, an alteration includes an insertion, deletion, or substitution of a nucleobase or amino acid.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1) in conjunction with a guide polynucleotide (e.g., guide RNA (gRNA)). Representative nucleic acid and protein sequences of base editors are provided in the Sequence Listing as SEQ ID NOs: 274-283.

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine or adenine deaminase activity, e.g., converting A•T to G•C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

By “base editing activity” is meant acting to chemically alter a base within a polynucleotide. In one embodiment, a first base is converted to a second base. In one embodiment, the base editing activity is cytidine deaminase activity, e.g., converting target C•G to T•A. In another embodiment, the base editing activity is adenosine deaminase activity, e.g., converting A•T to G•C.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnI nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following:

Glutamine CAG→TAG Stop codon
CAA→TAA

Arginine CGA→TGA

Tryptophan TGG→TGA
TGG→TAG
TGG→TAA

By “cytidine deaminase” is meant a polypeptide or fragment thereof capable of catalyzing a deamination reaction that converts an amino group to a carbonyl group. In one embodiment, the cytidine deaminase converts cytosine to uracil or 5-methylcytosine to thymine. PmCDA1 (SEQ ID NO: 41-42), which is derived from Petromyzon marinus (Petromyzon marinus cytosine deaminase 1, “PmCDA1”), AID (Activation-induced cytidine deaminase; AICDA) (Exemplary AID polypeptide sequences are provided in the Sequence Listing as SEQ ID NOs: 43-44, 1372, and 1374-1377), which is derived from a mammal (e.g., human, swine, bovine, horse, monkey etc.), and APOBEC are exemplary cytidine deaminases (Exemplary APOBEC polypeptide sequences are provided in the Sequence Listing as SEQ ID NOs: 1378-1416, 1421, and 1422. Further exemplary cytidine deaminase (CDA) sequences are provided in the Sequence Listing as SEQ ID NOs: 1373, 1417-1420. Additional exemplary cytidine deaminase sequences, including APOBEC polypeptide sequences, are provided in the Sequence Listing as SEQ ID NOs: 1378-1422. Some aspects of the disclosure provide base editor proteins and base editor systems that are capable of deaminating a cytosine in a nucleic acid molecule (e.g., DNA or RNA). In some embodiments the base editor protein or base editor system comprises a cytidine deaminase or cytosine deaminase that is capable of deaminating a cytidine to uridine in a nucleic acid molecule (e.g., DNA or RNA). It should be appreciated that any of the cytidine or cytosine deaminases provided herein include variants of naturally-occurring cytidine and cytosine deaminases. Such variants may be engineered to increase the efficiency of on-target deaminase activity of a base editor protein or system in a nucleic acid molecule (e.g., DNA or RNA).

The term “deaminase” or “deaminase domain,” as used herein, refers to a protein or enzyme that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include neurological diseases and opthalmic diseases.

By “effective amount” is meant the amount of an agent or active compound, e.g., a base editor as described herein, that is required to ameliorate the symptoms of a disease relative to an untreated patient or an individual without disease, i.e., a healthy individual, or is the amount of the agent or active compound sufficient to elicit a desired biological response. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

The term “exonuclease” refers to a protein or polypeptide capable of digesting a nucleic acid (e.g., RNA or DNA) from free ends.

The term “endonuclease” refers to a protein or polypeptide capable of catalyzing (e.g., cleaving) internal regions in a nucleic acid (e.g., DNA or RNA).

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “guide RNA” or “gRNA” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

By “tRNA” or “transfer RNA” is meant an RNA molecule comprising a secondary and/or tertiary structure that is capable of being cleaved in a cell. Cleavage may occur, for example, though an RNase, such as RNase P or RNase Z.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 84), KRPAATKKAGQAKKKK (SEQ ID NO: 85), KKTELQTTNAENKTKKL (SEQ ID NO: 86), KRGINDRNFWRGENGRKTR (SEQ ID NO: 87), RKSGKIAAIWKRPRK (SEQ ID NO: 88), PKKKRKV (SEQ ID NO: 89), or MDSLLMNRRKFLYQFKNVRWAKGRRETYLC (SEQ ID NO: 90).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (ψ). A “nucleotide” consists of a nucleobase, a five carbon sugar (either ribose or deoxyribose), and at least one phosphate group.

The terms “nucleic acid” and “nucleic acid molecule,” as used herein, refer to a compound comprising a nucleobase and an acidic moiety, e.g., a nucleoside, a nucleotide, or a polymer of nucleotides.

As used herein, the terms “oligonucleotide” and “polynucleotide” can be used interchangeably to refer to a polymer of nucleotides.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/CpfI, Cas12b/C2cl, Cas12c/C2c3, Cas12d/CasY, Cas12e/CasX, Cas12g, Cas12h, Cas12i, and Cas12j/Casϕ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/CpfI, Cas12b/C2cl, Cas12c/C2c3, Cas12d/CasY, Cas12e/CasX, Cas12g, Cas12h, Cas12i, Cas12j/Casϕ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 223, 230-232, 235-242, 246-256, and 285-294.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

As used herein, “obtaining” as in “obtaining an agent” includes synthesizing, purchasing, or otherwise acquiring the agent.

A “patient” or “subject” as used herein refers to a mammalian subject or individual diagnosed with, at risk of having or developing, or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease casing mutation”, “disease causing variant”, “deleterious mutation”, or “predisposing mutation” refers to a genetic alteration or mutation that increases an individual's susceptibility or predisposition to a certain disease or disorder. In some embodiments, the pathogenic mutation comprises at least one wild-type amino acid substituted by at least one pathogenic amino acid in a protein encoded by a gene.

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” are used with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (CsnI) from Streptococcus pyogenes.

The term “single nucleotide polymorphism (SNP)” is a variation in a single nucleotide that occurs at a specific position in the genome, where each variation is present to some appreciable degree within a population (e.g., >1%).

By “specifically binds” is meant a nucleic acid molecule, polypeptide, polypeptide/polynucleotide complex, compound, or molecule that recognizes and binds a polypeptide and/or nucleic acid molecule of the invention, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least 60%, 80%, 85%, 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

COBALT is used, for example, with the following parameters:

a) alignment parameters: Gap penalties-11,-1 and End-Gap penalties-5,-1,

b) CDD Parameters: Use RPS BLAST on; Blast E-value 0.003; Find Conserved columns and Recompute on, and

c) Query Clustering Parameters: Use query clusters on; Word Size 4; Max cluster distance 0.8; Alphabet Regular.

EMBOSS Needle is used, for example, with the following parameters:

a) Matrix: BLOSUM62;

b) GAP OPEN: 10;

c) GAP EXTEND: 0.5;

d) OUTPUT FORMAT: pair;

e) END GAP PENALTY: false;

f) END GAP OPEN: 10; and

g) END GAP EXTEND: 0.5.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In an embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein.

The term “target site” refers to a sequence within a nucleic acid molecule that is deaminated by a deaminase (e.g., cytidine or adenine deaminase) or a fusion protein comprising a deaminase (e.g., a dCas9-adenosine deaminase fusion protein or a base editor disclosed herein).

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a compositions as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. Including an inhibitor of uracil DNA glycosylase (UGI) in the base editor prevents base excision repair which changes the U back to a C. An exemplary UGI comprises an amino acid sequence as follows:

>sp|P14739|UNGI_BPPB2 Uracil-DNA
glycosylase inhibitor
(SEQ ID NO: 106)
MTNLSDIIEKETGKQLVIQESILMLPEEVEEVIGNKPESDILVHTAYDES
TDENVMLLTSDAPEYKPWALVIQDSNGENKIKML.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, within 2-fold of a value. Where particular values are described in the application and claims, unless otherwise stated, the term “about” means within an acceptable error range for the particular value should be assumed.

Reference in the specification to “certain embodiments,” “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### B. RECOMBINANT RABIES VIRUSES

Provided herein are recombinant rabies viruses that are useful for transducing a target cell. In one aspect, a recombinant rabies virus of the present disclosure comprises a rabies virus glycoprotein and a recombinant rabies virus genome. In certain embodiments, the recombinant rabies virus genome encodes a nucleic acid comprising a transgene. In certain embodiments, the recombinant rabies virus genome encodes a nucleic acid comprising a therapeutic transgene. As such, recombinant rabies viruses of the present disclosure can be employed in a method for transducing a target cell, wherein the recombinant rabies virus comprises a rabies virus glycoprotein and a recombinant rabies virus genome comprising a nucleic acid comprising a transgene (e.g., a therapeutic transgene). Upon transduction of the target cell, the transgene comprised within the recombinant rabies virus genome is expressed and a transgene product is produced.

Also known as Rabies lyssavirus, Rabies virus is a negative sense single stranded RNA virus of the Lyssavirus genus of the Rhabdoviridae family. Rabies virus has a cylindrical morphology, and the structure includes a lipoprotein envelope composed of glygoprotein G surrounding a helical ribonucleoprotein core. The rabies virus genome contains five genes that encode for proteins that promote transcription and replication of the genome and proteins that make up the structural components of the virus. The five genes are: the N gene encoding for a rabies virus nucleoprotein; the P gene encoding for a rabies virus phosphoprotein; the M gene encoding for a rabies virus matrix protein; the G gene encoding for a rabies virus glycoprotein; and the L gene encoding for a rabies virus polymerase. Viral genome RNA and the nucleoprotein together form a ribonucleoprotein that functions as a template for replication and transcription by the rabies virus polymerase (an RNA-dependent RNA polymerase).

In certain embodiments, a recombinant rabies virus genome of the present disclosure has one or more rabies virus genes removed. For example, the N gene, the P gene, the M gene, the L gene, and/or the G gene may be absent from the recombinant rabies virus genome. In certain embodiments, the recombinant rabies virus genome lacks a G gene encoding for a rabies virus glycoprotein or a functional variant thereof. Recombinant rabies virus genomes that lack a G gene encoding for a rabies virus glycoprotein prevents the virus from being able to endogenously produce glycoprotein. Because the glycoprotein is only required for the final steps of the viral life cycle, this deletion prevents the virus from spreading beyond initially infected cells, but it does not prevent the virus from completing the entirety of its replication cycle up to that point. In certain embodiments, the recombinant rabies virus genome lacks an L gene encoding for a rabies virus polymerase or a functional variant thereof. The L gene product is required both for transcription of viral genes and for replication of the viral genome, and deletion of the L gene may result in less cytotoxicity of a target transduced cell. See, e.g., Chatterjee et al., Nat. Neurosci. (2018) 21(4): 638-646, the disclosure of which is herein incorporated by reference in its entirety. In certain embodiments, the recombinant rabies virus genome lacks a G gene encoding for a rabies virus glycoprotein or a functional variant thereof, and lacks an L gene encoding for a rabies virus polymerase or a functional variant thereof.

It is readily appreciated by those of ordinary skill in the art that a recombinant rabies virus genome that lacks a rabies virus gene, as described herein, refers to a rabies virus genome that lacks all or a portion of the rabies virus gene. For example, a recombinant rabies virus genome that lacks a G gene may lack all or a portion of the G gene, wherein the portion of the G gene is required for the function of the G gene product. In certain embodiments, lacking a portion of the G gene that is required for the function of the G gene product may result in the production of a truncated, non-functional glycoprotein. In certain embodiments, a recombinant rabies virus genome that lacks an L gene may lack all or a portion of the L gene, wherein the portion of the L gene is required for the function of the L gene product. In certain embodiments, lacking a portion of the L gene that is required for the function of the L gene product may result in the production of a truncated, non-functional RNA-dependent RNA polymerase.

In certain embodiments, a recombinant rabies virus genome of the present disclosure encodes a nucleic acid comprising a transgene. In certain embodiments, the nucleic acid comprising a transgene replaces the one or more rabies virus genes that are removed, as described herein. For example, the nucleic acid comprising a transgene may replace all or a portion of a rabies virus gene. In certain embodiments, the nucleic acid comprising a transgene replaces all or a portion of a G gene, wherein the portion of the G gene is required for the function of the G gene product. In certain embodiments, the nucleic acid comprising a transgene replaces all or a portion of an L gene, wherein the portion of the L gene is required for the function of the L gene product. In certain embodiments, the nucleic acid comprising a transgene replaces all or a portion of an L gene, wherein the portion of the L gene is required for the function of the L gene product; and all or a portion of a G gene, wherein the portion of the G gene is required for the function of the G gene product.

In certain embodiments, a recombinant rabies virus genome of the present disclosure encodes a nucleic acid comprising a transgene, wherein the transgene replaces the one or more rabies virus genes that are removed, as described herein. In certain embodiments, the recombinant rabies virus genome comprises an N gene encoding for a rabies virus nucleoprotein or a functional variant thereof, a P gene encoding for a rabies virus phosphoprotein or a functional variant thereof, and/or an M gene encoding for a rabies virus matrix protein or a functional variant thereof.

Exemplary nucleic acid sequences of the N, P, M, L, and G genes, and the amino acid sequence of the gene products thereof are provided in Table 1.

TABLE 1
Exemplary sequences for N, P, M, L, and G

| SEQ ID | |
|---|---|
| NO: | Sequence |
| SEQ ID | atggatgccgacaagattgtattcaaagtcaataatcaggtggtctctttgaagc |
| NO: 4001 | ctgagattatcgtggatcaatatgagtacaagtaccctgccatcaaagatttgaa |
| N gene | aaagccctgtataaccctaggaaaggctcccgatttaaataaagcatacaagtca |
| (nucleic | gttttgtcaggcatgagcgccgccaaacttaatcctgacgatgtatgttcctatt |
| acid) | tggcagcggcaatgcagttttttgaggggacatgtccggaagactggaccagcta |
| | tggaattgtgattgcacgaaaaggagataagatcaccccaggttctctggtggag |
| | ataaaacgtactgatgtagaagggaattgggctctgacaggaggcatggaactga |
| | caagagaccccactgtccctgagcatgcgtccttagtcggtcttctcttgagtct |
| | gtataggttgagcaaaatatccgggcaaaacactggtaactataagacaaacatt |
| | gcagacaggatagagcagatttttgagacagccccttttgttaaaatcgtggaac |
| | accatactctaatgacaactcacaaaatgtgtgctaattggagtactataccaaa |
| | cttcagatttttggccggaacctatgacatgtttttctcccggattgagcatcta |
| | tattcagcaatcagagtgggcacagttgtcactgcttatgaagactgttcaggac |
| | tggtatcatttactgggttcataaaacaaatcaatctcaccgctagagaggcaat |
| | actatatttcttccacaagaactttgaggaagagataagaagaatgtttgagcca |
| | gggcaggagacagctgttcctcactcttatttcatccacttccgttcactaggct |
| | tgagtgggaaatctccttattcatcaaatgctgttggtcacgtgttcaatctcat |
| | tcactttgtaggatgctatatgggtcaagtcagatccctaaatgcaacggttatt |
| | gctgcatgtgctcctcatgaaatgtctgttctagggggctatctgggagaggaat |
| | tcttcgggaaagggacatttgaaagaagattcttcagagatgagaaagaacttca |
| | agaatacgaggcggctgaactgacaaagactgacgtagcactggcagatgatgga |
| | actgtcaactctgacgacgaggactacttttcaggtgaaaccagaagtccggagg |
| | ctgtttatactcgaatcatgatgaatggaggtcgactaaagagatctcacatacg |
| | gagatatgtctcagtcagttccaatcatcaagcccgtccaaactcattcgccgag |
| | tttctaaacaagacatattcgagtgactca |
| SEQ ID | ATGGATGCCGACAAGATTGTATTCAAAGTCAACAACCAGGTGGTGTCCCTGAAGC |
| NO: 4013 | CTGAGATCATCGTGGACCAGTACGAGTACAAGTACCCCGCCATCAAGGACCTGAA |
| N gene | GAAGCCCTGTATCACCCTGGGCAAAGCCCCTGACCTGAACAAAGCCTACAAGAGC |
| codon | GTGCTGAGCGGCATGTCTGCCGCCAAGCTGAACCCTGATGACGTGTGCTCTTATC |
| optimzed | TGGCCGCTGCCATGCAGTTCTTCGAGGGCACATGTCCCGAGGACTGGACCAGCTA |
| (nucleic | TGGAATCGTGATCGCCCGGAAGGGCGATAAGATCACACCTGGCAGCCTGGTGGAA |
| acid) | ATCAAGAGAACCGACGTGGAAGGCAACTGGGCCCTGACAGGTGGCATGGAACTGA |
| | CCAGAGATCCCACCGTGCCTGAGCACGCTTCTCTTGTTGGACTGCTGCTGAGCCT |
| | GTACCGGCTGTCTAAGATCAGCGGACAGAACACCGGCAACTACAAGACCAATATC |
| | GCCGACCGGATCGAGCAGATTTTCGAGACAGCCCCTTTCGTGAAGATCGTGGAAC |
| | ACCACACACTGATGACCACACACAAGATGTGCGCCAACTGGTCTACAATCCCCAA |
| | CTTCAGATTCCTGGCCGGCACCTACGACATGTTCTTCAGCAGAATCGAGCACCTG |
| | TACTCTGCCATCAGAGTGGGCACAGTCGTGACCGCCTACGAGGATTGCTCTGGCC |
| | TGGTGTCCTTCACCGGCTTCATCAAGCAGATCAACCTGACCGCCAGAGAGGCCAT |
| | CCTGTACTTTTTCCACAAGAACTTCGAGGAAGAGATCCGGCGGATGTTCGAGCCC |
| | GGACAAGAAACAGCCGTGCCTCACAGCTACTTCATCCACTTCAGAAGCCTGGGCC |
| | TGTCCGGCAAGAGCCCCTACTCTTCTAATGCCGTGGGCCACGTGTTCAACCTGAT |
| | CCACTTCGTGGGCTGCTACATGGGCCAAGTGCGGAGCCTGAATGCCACAGTGATT |
| | GCCGCCTGTGCTCCCCACGAAATGTCTGTGCTCGGAGGCTATCTGGGCGAAGAGT |
| | TCTTTGGCAAGGGCACCTTCGAGCGGAGATTCTTCCGGGACGAGAAAGAGCTGCA |
| | AGAGTACGAGGCCGCCGAGCTGACCAAAACAGATGTGGCCCTGGCCGATGACGGC |
| | ACCGTGAACTCTGACGACGAGGACTACTTCAGCGGCGAGACTAGAAGCCCCGAGG |
| | CCGTGTATACCCGGATTATGATGAATGGCGGCAGGCTGAAGCGGAGCCACATTCG |
| | GAGATACGTGTCCGTGTCCAGCAACCATCAGGCCAGACCTAACAGCTTCGCCGAG |
| | TTCCTGAACAAGACCTACTCGAGTGACTCATAA |
| SEQ ID | MDADKIVFKVNNQVVSLKPEIIVDQYEYKYPAIKDLKKPCITLGKAPDLNKAYKS |
| NO: 4002 | VLSGMSAAKLNPDDVCSYLAAAMQFFEGTCPEDWTSYGIVIARKGDKITPGSLVE |
| N gene | IKRTDVEGNWALTGGMELTRDPTVPEHASLVGLLLSLYRLSKISGQNTGNYKTNI |
| (amino | ADRIEQIFETAPFVKIVEHHTLMTTHKMCANWSTIPNFRFLAGTYDMFFSRIEHL |
| acid) | YSAIRVGTVVTAYEDCSGLVSFTGFIKQINLTAREAILYFFHKNFEEEIRRMFEP |
| | GQETAVPHSYFIHFRSLGLSGKSPYSSNAVGHVFNLIHFVGCYMGQVRSLNATVI |
| | AACAPHEMSVLGGYLGEEFFGKGTFERRFFRDEKELQEYEAAELTKTDVALADDG |
| | TVNSDDEDYFSGETRSPEAVYTRIMMNGGRLKRSHIRRYVSVSSNHQARPNSFAE |
| | FLNKTYSSDS |
| SEQ ID | ctcgatcctggagaggtctatgatgaccctattgacccaatcgagttagaggctg |
| NO: 4003 | aacccagaggaacccccattgtccccaacatcttgaggaactctgactacaatct |
| L gene | caactctcdttgatagaagatcctgctagactaatgttagaatggttaaaaacag |
| (nucleic | ggaatagaccttatcggatgactctaacagacaattgctccaggtctttcagagt |
| acid) | tttgaaagattatttcaagaaggtagatttgggttctctcaaggtgggcggaatg |
| | gctgcacagtcaatgatttctctctggttatatggtgcccactctgaatccaaca |
| | ggagccggagatgtataacagacttggcccatttctattccaagtcgtcccccat |
| | agagaagctgttgaatctcacgctaggaaatagagggctgagaatccccccagag |
| | ggagtgttaagttgccttgagagggttgattatgataatgcatttggaaggtatc |
| | ttgccaacacgtattcctcttacttgttcttccatgtaatcaccttatacatgaa |
| | cgccctagactgggatgaagaaaagaccatcctagcattatggaaagatttaacc |
| | tcagtggacatcgggaaggacttggtaaagttcaaagaccaaatatggggactgc |
| | tgatcgtgacaaaggactttgtttactcccaaagttccaattgtctttttgacag |
| | aaactacacacttatgctaaaagatcttttcttgtctcgcttcaactccttaatg |
| | gtcttgctctctcccccagagccccgatactcagatgacttgatatctcaactat |
| | gccagctgtacattgctggggatcaagtcttgtctatgtgtggaaactccggcta |
| | tgaagtcatcaaaatattggagccatatgtcgtgaatagtttagtccagagagca |
| | gaaaagtttaggcctctcattcattccttgggagactttcctgtatttataaaag |
| | acaaggtaagtcaacttgaagagacgttcggtccctgtgcaagaaggttctttag |
| | ggctctggatcaattcgacaacatacatgacttggtttttgtgtttggctgttac |
| | aggcattgggggcacccatatatagattatcgaaagggtctgtcaaaactatatg |
| | atcaggttcaccttaaaaaaatgatagataagtcctaccaggagtgcttagcaag |
| | cgacctagccaggaggatccttagatggggttttgataagtactccaagtggtat |
| | ctggattcaagattcctagcccgagaccacccdtgactccttatatcaaaaccca |
| | aacatggccacccaaacatattgtagacttggtgggggatacatggcacaagctc |
| | ccgatcacgcagatctttgagattcctgaatcaatggatccgtcagaaatattgg |
| | atgacaaatcacattctttcaccagaacgagactagcttcttggctgtcagaaaa |
| | ccgaggggggcctgttcctagcgaaaaagttattatcacggccctgtctaagccg |
| | cctgtcaatccccgagagtttctgaggtctatagacctcggaggattgccagatg |
| | aagacttgataattggcctcaagccaaaggaacgggaattgaagattgaaggtcg |
| | attctttgctctaatgtcatggaatctaagattgtattttgtcatcactgaaaaa |
| | ctcttggccaactacatcttgccactttttgacgcgctgactatgacagacaacc |
| | tgaacaaggtgtttaaaaagctgatcgacagggtcaccgggcaagggcttttgga |
| | ctattcaagggtcacatatgcatttcacctggactatgaaaagtggaacaaccat |
| | caaagattagagtcaacagaggatgtattttctgtcctagatcaagtgtttggat |
| | tgaagagagtgttttctagaacacacgagttttttcaaaaggcctggatctatta |
| | ttcagacagatcagacctcatcgggttacgggaggatcaaatatactgcttagat |
| | gcgtccaacggcccaacctgttggaatggccaggatggcgggctagaaggcttac |
| | ggcagaagggctggagtctagtcagcttattgatgatagatagagaatctcaaat |
| | caggaacacaagaaccaaaatactagctcaaggagacaaccaggttttatgtccg |
| | acatacatgttgtcgccagggctatctcaagaggggctcctctatgaattggaga |
| | gaatatcaaggaatgcactttcgatatacagagccgtcgaggaaggggcatctaa |
| | gctagggctgatcatcaagaaagaagagaccatgtgtagttatgacttcctcatc |
| | tatggaaaaacccctttgtttagaggtaacatattggtgcctgagtccaaaagat |
| | gggccagagtctcttgcgtctctaatgaccaaatagtcaacctcgccaatataat |
| | gtcgacagtgtccaccaatgcgctaacagtggcacaacactctcaatctttgatc |
| | aaaccgatgagggattttctgctcatgtcagtacaggcagtctttcactacctgc |
| | tatttagcccaatcttaaagggaagagtttacaagattctgagcgctgaagggga |
| | gagctttctcctagccatgtcaaggataatctatctagatccttctttgggaggg |
| | atatctggaatgtccctcggaagattccatatacgacagttctcagaccctgtct |
| | ctgaagggttatccttctggagagagatctggttaagctcccaagagtcctggat |
| | tcacgcgttgtgtcaagaggctggaaacccagatcttggagagagaacactcgag |
| | agcttcactcgccttctagaagatccgaccaccttaaatatcagaggaggggcca |
| | gtcctaccattctactcaaggatgcaatcagaaaggctttatatgacgaggtgga |
| | caaggtggaaaattcagagtttcgagaggcaatcctgttgtccaagacccataga |
| | gataattttatactcttcttaatatctgttgagcctctgtttcctcgatttctca |
| | gtgagctattcagttcgtcttttttgggaatccccgagtcaatcattggattgat |
| | acaaaactcccgaacgataagaaggcagtttagaaagagtctctcaaaaacttta |
| | gaagaatccttctacaactcagagatccacgggattagtcggatgacccagacac |
| | ctcagagggttgggggggtgtggccttgctcttcagagagggcagatctacttag |
| | ggagatctcttggggaagaaaagtggtaggcacgacagttcctcacccttctgag |
| | atgttgggattacttcccaagtcctctatttcttgcacttgtggagcaacaggag |
| | gaggcaatcctagagtttctgtatcagtactcccgtcctttgatcagtcattttt |
| | ttcacgaggccccctaaagggatacttgggctcgtccacctctatgtcgacccag |
| | ctattccatgcatgggaaaaagtcactaatgttcatgtggtgaagagagctctat |
| | cgttaaaagaatctataaactggttcattactagagattccaacttggctcaagc |
| | tctaattaggaacattatgtctctgacaggccctgatttccctctagaggaggcc |
| | cctgtcttcaaaaggacggggtcagccttgcataggttcaagtctgccagataca |
| | gcgaaggagggtattcttctgtctgcccgaacctcctctctcatatttctgttag |
| | tacagacaccatgtctgatttgacccaagacgggaagaactacgatttcatgttc |
| | cagccattgatgctttatgcacagacatggacatcagagctggtacagagagaca |
| | caaggctaagagactctacgtttcattggcacctccgatgcaacaggtgtgtgag |
| | acccattgacgacgtgaccctggagacctctcagatcttcgagtttccggatgtg |
| | tcgaaaagaatatccagaatggtttctggggctgtgcctcacttccagaggcttc |
| | ccgatatccgtctgagaccaggagattttgaatctctaagcggtagagaaaagtc |
| | tcaccatatcggatcagctcaggggctcttatactcaatcttagtggcaattcac |
| | gactcaggatacaatgatggaaccatcttccctgtcaacatatacggcaaggttt |
| | cccctagagactatttgagagggctcgcaaggggagtattgataggatcctcgat |
| | ttgcttcttgacaagaatgacaaatatcaatattaatagacctcttgaattggtc |
| | tcaggggtaatctcatatattctcctgaggctagataaccatccctccttgtaca |
| | taatgctcagagaaccgtctcttagaggagagatattttctatccctcagaaaat |
| | ccccgccgcttatccaaccactatgaaagaaggcaacagatcaatcttgtgttat |
| | ctccaacatgtgctacgctatgagcgagagataatcacggcgtctccagagaatg |
| | actggctatggatcttttcagactttagaagtgccaaaatgacgtacctatccct |
| | cattacttaccagtctcatcttctactccagagggttgagagaaacctatctaag |
| | agtatgagagataacctgcgacaattgagttctttgatgaggcaggtgctgggcg |
| | ggcacggagaagataccttagagtcagacgacaacattcaacgactgctaaaaga |
| | ctctttacgaaggacaagatgggtggatcaagaggtgcgccatgcagctagaacc |
| | atgactggagattacagccccaacaagaaggtgtcccgtaaggtaggatgttcag |
| | aatgggtctgctctgctcaacaggttgcagtctctacctcagcaaacccggcccc |
| | tgtctcggagcttgacataagggccctctctaagaggttccagaaccctttgatc |
| | tcgggcttgagagtggttcagtgggcaaccggtgctcattataagcttaagccta |
| | ttctagatgatctcaatgttttcccatctctctgccttgtagttggggacgggtc |
| | aggggggatatcaagggcagtcctcaacatgtttccagatgccaagcttgtgttc |
| | aacagtcttttagaggtgaatgacctgatggcttccggaacacatccactgcctc |
| | cttcagcaatcatgaggggaggaaatgatatcgtctccagagtgatagatcttga |
| | ctcaatctgggaaaaaccgtccgacttgagaaacttggcaacctggaaatacttc |
| | cagtcagtccaaaagcaggtcaacatgtcctatgacctcattatttgcgatgcag |
| | aagttactgacattgcatctatcaaccggatcaccctgttaatgtccgattttgc |
| | attgtctatagatggaccactctatttggtcttcaaaacttatgggactatgcta |
| | gtaaatccaaactacaaggctattcaacacctgtcaagagcgttcccctcggtca |
| | cagggtttatcacccaagtaacttcgtctttttcatctgagctctacctccgatt |
| | ctccaaacgagggaagtttttcagagatgctgagtacttgacctcttccaccctt |
| | cgagaaatgagccttgtgttattcaattgtagcagccccaagagtgagatgcaga |
| | gagctcgttccttgaactatcaggatcttgtgagaggatttcctgaagaaatcat |
| | atcaaatccttacaatgagatgatcataactctgattgacagtgatgtagaatct |
| | tttctagtccacaagatggttgatgatcttgagttacagaggggaactctgtcta |
| | aagtggctatcattatagccatcatgatagttttctccaacagagtcttcaacgt |
| | ttccaaacccctaactgacccctcgttctatccaccgtctgatcccaaaatcctg |
| | aggcacttcaacatatgttgcagtactatgatgtatctatctactgctttaggtg |
| | acgtccctagcttcgcaagacttcacgacctgtataacagacctataacttatta |
| | cttcagaaagcaagtcattcgagggaacgtttatctatcttggagttggtccaac |
| | gacacctcagtgttcaaaagggtagcctgtaattctagcctgagtctgtcatctc |
| | actggatcaggttgatttacaagatagtgaagactaccagactcgttggcagcat |
| | caaggatctatccagagaagtggaaagacaccttcataggtacaacaggtggatc |
| | accctagaggatatcagatctagatcatccctactagactacagttgcctg |
| SEQ ID | ATGCTCGATCCTGGAGAGGTCTATGATGACCCCATCGATCCTATCGAGCTGGAAG |
| NO: 4014 | CCGAGCCTAGAGGCACACCCATCGTGCCCAACATCCTGCGGAACAGCGACTACAA |
| L gene | CCTGAACAGCCCTCTGATCGAGGACCCCGCCAGACTGATGCTGGAATGGCTGAAA |
| codon | ACCGGCAACAGACCCTACCGGATGACCCTGACCGATAACTGCTCCCGGTCCTTCA |
| optimized | GGGTGCTGAAGGACTACTTCAAGAAGGTGGACCTGGGCAGCCTGAAAGTCGGAGG |
| (nucleic | AATGGCCGCTCAGAGCATGATCAGCCTGTGGCTGTATGGCGCCCACAGCGAGAGC |
| acid) | AACAGATCCAGAAGATGCATCACCGATCTGGCCCACTTCTACAGCAAGAGCAGCC |
| | CCATCGAGAAGCTGCTGAATCTGACCCTGGGCAACCGCGGCCTGAGAATACCTCC |
| | TGAAGGCGTGCTGAGCTGCCTGGAAAGAGTGGACTACGACAACGCCTTCGGCAGA |
| | TACCTGGCCAACACCTACAGCAGCTACCTGTTCTTCCACGTGATCACCCTGTATA |
| | TGAACGCCCTGGACTGGGACGAAGAGAAAACCATTCTGGCCCTGTGGAAGGACCT |
| | GACCTCTGTGGACATCGGCAAGGACCTGGTCAAGTTCAAGGACCAGATTTGGGGC |
| | CTGCTGATCGTGACCAAGGACTTCGTGTACTCCCAGAGCAGCAACTGCCTGTTCG |
| | ACCGGAACTACACCCTGATGCTGAAAGACCTGTTCCTGAGCCGGTTCAACAGCCT |
| | GATGGTGCTGCTGTCTCCTCCAGAGCCTAGATACAGCGACGACCTGATCTCCCAG |
| | CTGTGCCAGCTGTATATTGCCGGCGATCAGGTGCTGAGCATGTGCGGCAATAGCG |
| | GCTACGAAGTGATCAAGATCCTGGAACCTTACGTCGTGAACAGCCTGGTGCAGCG |
| | GGCCGAGAAGTTCAGACCACTGATTCACAGCCTGGGCGACTTCCCCGTGTTCATC |
| | AAGGACAAGGTGTCCCAGCTGGAAGAGACATTCGGCCCCTGCGCCAGAAGATTCT |
| | TCAGAGCCCTGGACCAGTTCGACAACATCCACGACCTGGTGTTCGTGTTCGGCTG |
| | CTACAGACACTGGGGACACCCCTACATCGACTACAGAAAGGGCCTGAGCAAGCTG |
| | TACGACCAGGTTCACCTGAAGAAGATGATCGACAAGAGCTACCAAGAGTGCCTGG |
| | CCAGCGACCTGGCCAGACGTATTCTGAGATGGGGCTTCGACAAGTACAGCAAGTG |
| | GTATCTGGACAGCCGGTTCCTGGCTCGGGATCACCCTCTGACTCCCTACATCAAG |
| | ACCCAGACCTGGCCTCCTAAGCACATCGTGGATCTCGTGGGCGACACCTGGCACA |
| | AGCTGCCCATCACACAGATTTTCGAGATCCCCGAGAGCATGGACCCCAGCGAGAT |
| | TCTGGACGACAAGTCCCACAGCTTCACCCGGACAAGACTGGCCTCTTGGCTGAGC |
| | GAGAATAGAGGCGGACCTGTGCCTAGCGAGAAAGTGATCATCACCGCTCTGTCCA |
| | AGCCTCCAGTGAACCCCAGAGAGTTCCTGCGGTCTATCGATCTCGGCGGCCTGCC |
| | TGATGAGGACCTGATCATTGGCCTGAAGCCTAAAGAGCGCGAGCTGAAGATCGAG |
| | GGCAGATTCTTCGCCCTGATGAGCTGGAACCTGCGGCTGTACTTTGTGATCACCG |
| | AGAAACTGCTGGCCAACTACATCCTGCCTCTGTTCGACGCCCTGACCATGACCGA |
| | CAATCTGAACAAGGTGTTCAAGAAACTGATCGACAGAGTGACCGGCCAGGGACTG |
| | CTGGACTACAGCAGAGTGACATACGCCTTCCACCTGGATTACGAGAAGTGGAACA |
| | ACCACCAGCGGCTGGAAAGCACCGAGGACGTGTTCTCTGTGCTGGACCAGGTGTT |
| | CGGCCTGAAGAGAGTGTTCAGCAGAACCCACGAGTTCTTCCAGAAAGCCTGGATC |
| | TACTACAGCGACCGCAGCGATCTGATCGGACTGAGAGAGGACCAAATCTACTGCC |
| | TGGACGCCAGCAATGGCCCTACCTGTTGGAATGGACAGGACGGCGGACTGGAAGG |
| | ACTGAGACAGAAAGGCTGGTCCCTGGTGTCCCTGCTGATGATTGACAGAGAGAGC |
| | CAGATCAGAAACACGCGGACCAAGATTCTGGCTCAGGGCGACAACCAGGTGCTGT |
| | GCCCTACCTATATGCTGAGCCCTGGCCTGTCTCAAGAGGGGCTGCTGTACGAACT |
| | GGAACGGATCAGCAGAAACGCCCTGTCCATCTATAGAGCCGTGGAAGAGGGCGCC |
| | TCTAAGCTGGGCCTGATCATCAAGAAAGAAGAGACAATGTGCAGCTACGACTTCC |
| | TGATCTACGGCAAGACCCCTCTGTTCCGGGGCAACATTCTGGTGCCCGAGTCCAA |
| | GAGATGGGCCAGAGTGTCCTGCGTGTCCAACGACCAGATCGTGAATCTGGCCAAT |
| | ATCATGTCCACCGTGTCCACCAACGCTCTGACAGTGGCCCAGCACAGCCAGTCTC |
| | TGATCAAGCCTATGCGGGACTTCCTGCTCATGTCCGTGCAGGCCGTGTTCCACTA |
| | CCTGCTGTTCAGCCCTATCCTGAAGGGCAGAGTGTATAAGATCCTGAGCGCCGAG |
| | GGCGAGAGCTTCCTGCTTGCCATGAGCCGGATCATCTATCTGGACCCTAGCCTCG |
| | GCGGCATCAGCGGCATGTCTCTGGGCAGATTTCACATCCGGCAGTTCAGCGACCC |
| | TGTGTCCGAGGGCCTGTCCTTTTGGAGAGAAATCTGGCTGTCTAGCCAAGAGAGC |
| | TGGATTCACGCCCTGTGCCAAGAAGCCGGCAATCCCGATCTGGGCGAGAGAACCC |
| | TGGAATCCTTCACCAGACTGCTTGAGGACCCCACCACTCTGAACATCAGAGGCGG |
| | AGCCTCTCCAACCATCCTGCTCAAGGACGCCATCCGCAAGGCCCTGTATGACGAG |
| | GTGGACAAAGTGGAAAACAGCGAGTTCAGAGAGGCCATTCTGCTGAGCAAGACCC |
| | ACCGGGACAACTTCATCCTGTTCCTCATCTCCGTGGAACCCCTGTTTCCTAGATT |
| | CCTGTCCGAGCTGTTCTCCAGCTCCTTCCTGGGCATCCCTGAGTCCATCATCGGC |
| | CTGATCCAGAACTCCCGGACCATCCGCAGACAGTTCAGAAAGAGCCTGTCTAAGA |
| | CCCTGGAAGAGTCCTTCTACAACAGCGAAATCCACGGCATCTCCCGGATGACACA |
| | GACCCCTCAAAGAGTCGGCGGCGTCTGGCCTTGTTCTAGCGAAAGAGCCGACCTG |
| | CTGAGAGAGATCAGCTGGGGCAGAAAGGTCGTGGGCACCACAGTGCCTCATCCAA |
| | GCGAAATGCTGGGCCTCCTGCCTAAGAGCAGCATCAGCTGTACCTGTGGCGCTAC |
| | CGGCGGAGGCAATCCTAGAGTGTCTGTGTCTGTGCTGCCCAGCTTCGACCAGAGC |
| | TTCTTCAGCAGAGGACCTCTGAAGGGCTACCTGGGCTCTAGCACCAGCATGAGCA |
| | CCCAGCTGTTTCACGCCTGGGAAAAAGTGACCAATGTGCACGTGGTCAAGAGAGC |
| | CCTGTCTCTGAAAGAGAGCATCAACTGGTTCATCACGCGGGACAGCAATCTGGCA |
| | CAGGCCCTGATTCGGAACATCATGTCCCTGACTGGCCCTGACTTTCCACTGGAAG |
| | AGGCCCCTGTGTTCAAGCGCACAGGATCTGCCCTGCACAGATTCAAGAGCGCCAG |
| | ATACTCCGAAGGCGGCTACAGCTCCGTGTGTCCCAATCTGCTGTCCCACATCTCT |
| | GTGTCCACCGACACCATGTCCGATCTGACCCAGGACGGCAAGAACTACGATTTCA |
| | TGTTCCAGCCTCTCATGCTGTACGCCCAGACATGGACAAGCGAGCTGGTGCAGAG |
| | AGACACCCGGCTGAGAGATAGCACCTTCCACTGGCACCTGAGGTGCAACAGATGC |
| | GTGCGGCCCATCGACGACGTGACACTGGAAACCTCTCAGATATTCGAGTTCCCAG |
| | ACGTGTCCAAGCGGATCAGCCGAATGGTGTCTGGCGCCGTGCCTCACTTCCAAAG |
| | ACTGCCCGACATCAGACTGCGGCCAGGCGATTTTGAGAGCCTGAGCGGCAGAGAG |
| | AAGTCCCACCACATCGGATCTGCACAGGGCCTGCTCTACTCTATCCTGGTGGCCA |
| | TCCACGATTCCGGCTACAACGACGGCACCATCTTTCCCGTGAACATCTACGGAAA |
| | AGTGTCCCCACGGGACTACCTGAGAGGACTGGCTAGAGGGGTGCTGATCGGCAGC |
| | AGCATCTGCTTTCTGACCAGAATGACCAACATCAACATCAATAGGCCCCTGGAAC |
| | TGGTGTCCGGCGTGATCAGCTATATCCTGCTGCGGCTGGACAATCACCCCAGCCT |
| | GTATATCATGCTGAGGGAACCCAGCCTGCGGGGCGAGATTTTTAGCATCCCTCAG |
| | AAGATCCCTGCCGCCTATCCTACCACCATGAAGGAAGGCAATCGGAGCATCCTGT |
| | GCTACCTCCAGCATGTGCTGAGATACGAGCGGGAAATCATTACCGCCTCTCCAGA |
| | GAACGATTGGCTGTGGATCTTTAGCGACTTCCGCAGCGCCAAGATGACCTACCTG |
| | AGCCTGATCACCTACCAGAGCCATCTGCTGCTCCAGAGAGTGGAACGGAACCTGT |
| | CCAAGAGCATGAGGGACAACCTGAGGCAGCTGTCCTCTCTGATGAGACAGGTGCT |
| | CGGAGGACACGGCGAGGATACACTGGAATCTGACGACAATATCCAGCGGCTCCTG |
| | AAAGACAGCCTGAGAAGAACCAGATGGGTTGACCAAGAAGTGCGCCACGCCGCCA |
| | GAACAATGACCGGCGATTACAGCCCCAACAAGAAAGTGTCCAGAAAAGTGGGCTG |
| | CTCCGAGTGGGTCTGCTCTGCTCAGCAAGTTGCCGTGTCTACCAGCGCCAATCCT |
| | GCACCAGTTTCCGAGCTGGACATTAGAGCCCTGAGCAAACGGTTCCAGAATCCTC |
| | TGATCTCTGGCCTGAGAGTGGTGCAGTGGGCTACAGGCGCCCACTACAAGCTGAA |
| | GCCCATCCTGGACGATCTGAACGTGTTCCCTAGCCTGTGTCTGGTCGTCGGAGAT |
| | GGATCTGGCGGAATCAGCAGAGCCGTGCTGAATATGTTCCCCGACGCCAAGCTGG |
| | TGTTCAATAGCCTGCTGGAAGTGAACGATCTGATGGCCAGCGGCACACACCCTCT |
| | GCCACCAAGCGCAATTATGAGAGGCGGCAACGACATCGTGTCCAGAGTGATCGAC |
| | CTGGACTCCATCTGGGAGAAGCCCTCCGACCTGAGAAACCTGGCCACCTGGAAGT |
| | ACTTTCAGAGCGTGCAGAAACAAGTGAACATGAGCTACGACCTCATCATCTGCGA |
| | CGCCGAAGTGACCGACATTGCCTCCATCAACAGAATCACACTGCTGATGTCCGAC |
| | TTCGCTCTGAGCATCGACGGCCCTCTGTATCTGGTGTTTAAGACCTACGGCACAA |
| | TGCTCGTGAACCCTAACTACAAGGCCATCCAGCACCTCAGCAGGGCCTTTCCAAG |
| | CGTGACCGGCTTCATCACCCAAGTGACCTCCAGCTTCAGCAGCGAGCTGTATCTG |
| | CGGTTCAGCAAGCGGGGCAAGTTCTTCAGGGACGCCGAGTACCTGACCAGCAGCA |
| | CACTGAGAGAAATGAGTCTGGTGCTGTTCAACTGCTCTAGCCCCAAGAGCGAGAT |
| | GCAGAGAGCTAGAAGCCTGAACTACCAGGACCTCGTGCGGGGCTTCCCCGAAGAG |
| | ATCATCTCTAACCCCTACAACGAGATGATCATTACCCTGATCGACTCCGACGTCG |
| | AGAGCTTTCTGGTGCACAAGATGGTGGACGACCTGGAACTTCAGAGGGGCACACT |
| | GTCCAAGGTGGCCATTATCATTGCCATTATGATCGTGTTCTCCAACCGGGTTTTC |
| | AATGTCTCCAAGCCACTGACAGACCCCAGCTTCTACCCTCCTAGCGACCCAAAGA |
| | TCCTGCGGCACTTCAACATCTGTTGTAGCACCATGATGTACCTGAGCACCGCACT |
| | GGGAGATGTGCCATCCTTTGCCAGACTGCACGACCTGTATAACAGACCCATCACC |
| | TACTACTTTCGGAAGCAAGTGATCCGCGGCAACGTGTACCTGTCCTGGTCTTGGA |
| | GCAACGACACCAGCGTGTTCAAAAGAGTGGCCTGCAACAGCTCTCTGTCCCTGGC |
| | AGCAGCCACTGGATCAGACTGATCTACAAGATCGTCAAGACCACACGGCTCGTGG |
| | AGCATCAAGGATCTGAGTAGAGAGGTGGAAAGGCATCTGCATCGGTACAATCGGT |
| | GGATCACACTTGAGGACATCCGGTCCAGATCATCCCTACTAGACTACAGTTGCCT |
| | GTGA |
| SEQ ID | LDPGEVYDDPIDPIELEAEPRGTPIVPNILRNSDYNLNSPLIEDPARLMLEWLKT |
| NO: 4004 | GNRPYRMTLTDNCSRSFRVLKDYFKKVDLGSLKVGGMAAQSMISLWLYGAHSESN |
| L gene | RSRRCITDLAHFYSKSSPIEKLLNLTLGNRGLRIPPEGVLSCLERVDYDNAFGRY |
| (amino | LANTYSSYLFFHVITLYMNALDWDEEKTILALWKDLTSVDIGKDLVKFKDQIWGL |
| acid) | LIVTKDFVYSQSSNCLFDRNYTLMLKDLFLSRFNSLMVLLSPPEPRYSDDLISQL |
| | CQLYIAGDQVLSMCGNSGYEVIKILEPYVVNSLVQRAEKFRPLIHSLGDFPVFIK |
| | DKVSQLEETFGPCARRFFRALDQFDNIHDLVFVFGCYRHWGHPYIDYRKGLSKLY |
| | DQVHLKKMIDKSYQECLASDLARRILRWGFDKYSKWYLDSRFLARDHPLTPYIKT |
| | QTWPPKHIVDLVGDTWHKLPITQIFEIPESMDPSEILDDKSHSFTRTRLASWLSE |
| | NRGGPVPSEKVIITALSKPPVNPREFLRSIDLGGLPDEDLIIGLKPKERELKIEG |
| | RFFALMSWNLRLYFVITEKLLANYILPLFDALTMTDNLNKVFKKLIDRVTGQGLL |
| | DYSRVTYAFHLDYEKWNNHQRLESTEDVFSVLDQVFGLKRVFSRTHEFFQKAWIY |
| | YSDRSDLIGLREDQIYCLDASNGPTCWNGQDGGLEGLRQKGWSLVSLLMIDRESQ |
| | IRNTRTKILAQGDNQVLCPTYMLSPGLSQEGLLYELERISRNALSIYRAVEEGAS |
| | KLGLIIKKEETMCSYDFLIYGKTPLFRGNILVPESKRWARVSCVSNDQIVNLANI |
| | MSTVSTNALTVAQHSQSLIKPMRDFLLMSVQAVFHYLLFSPILKGRVYKILSAEG |
| | ESFLLAMSRIIYLDPSLGGISGMSLGRFHIRQFSDPVSEGLSFWREIWLSSQESW |
| | IHALCQEAGNPDLGERTLESFTRLLEDPTTLNIRGGASPTILLKDAIRKALYDEV |
| | DKVENSEFREAILLSKTHRDNFILFLISVEPLFPRFLSELFSSSFLGIPESIIGL |
| | IQNSRTIRRQFRKSLSKTLEESFYNSEIHGISRMTQTPQRVGGVWPCSSERADLL |
| | REISWGRKVVGTTVPHPSEMLGLLPKSSISCTCGATGGGNPRVSVSVLPSFDQSF |
| | FSRGPLKGYLGSSTSMSTQLFHAWEKVTNVHVVKRALSLKESINWFITRDSNLAQ |
| | ALIRNIMSLTGPDFPLEEAPVFKRTGSALHRFKSARYSEGGYSSVCPNLLSHISV |
| | STDTMSDLTQDGKNYDFMFQPLMLYAQTWTSELVQRDTRLRDSTFHWHLRCNRCV |
| | RPIDDVTLETSQIFEFPDVSKRISRMVSGAVPHFQRLPDIRLRPGDFESLSGREK |
| | SHHIGSAQGLLYSILVAIHDSGYNDGTIFPVNIYGKVSPRDYLRGLARGVLIGSS |
| | ICFLTRMTNININRPLELVSGVISYILLRLDNHPSLYIMLREPSLRGEIFSIPQK |
| | IPAAYPTTMKEGNRSILCYLQHVLRYEREIITASPENDWLWIFSDFRSAKMTYLS |
| | LITYQSHLLLQRVERNLSKSMRDNLRQLSSLMRQVLGGHGEDTLESDDNIQRLLK |
| | DSLRRTRWVDQEVRHAARTMTGDYSPNKKVSRKVGCSEWVCSAQQVAVSTSANPA |
| | PVSELDIRALSKRFQNPLISGLRVVQWATGAHYKLKPILDDLNVFPSLCLVVGDG |
| | SGGISRAVLNMFPDAKLVFNSLLEVNDLMASGTHPLPPSAIMRGGNDIVSRVIDL |
| | DSIWEKPSDLRNLATWKYFQSVQKQVNMSYDLIICDAEVTDIASINRITLLMSDF |
| | ALSIDGPLYLVFKTYGTMLVNPNYKAIQHLSRAFPSVTGFITQVTSSFSSELYLR |
| | FSKRGKFFRDAEYLTSSTLREMSLVLFNCSSPKSEMQRARSLNYQDLVRGFPEEI |
| | ISNPYNEMIITLIDSDVESFLVHKMVDDLELQRGTLSKVAIIIAIMIVFSNRVFN |
| | VSKPLTDPSFYPPSDPKILRHFNICCSTMMYLSTALGDVPSFARLHDLYNRPITY |
| | YFRKQVIRGNVYLSWSWSNDTSVFKRVACNSSLSLSSHWIRLIYKIVKTTRLVGS |
| | IKDLSREVERHLHRYNRWITLEDIRSRSSLLDYSCL |
| SEQ ID | ttctagaagcagagaggaatctttgtcctcttcggacctttgtgtctgaagagac |
| NO: 4005 | atgtcagaccatagttgacatgctctcgggttcatgttgatacaccagactctgc |
| M gene | cctggatatgacactgttttgcaatcactcttatttgcaatccgacgaactcagt |
| (nucleic | atcatcatcccaagtgatctcctgagagtattccaactcctccccttcaagaggg |
| acid) | cccctggaatcagcccactggaagataaaggttctcctcaatttgtatacccagt |
| | tcaggccctcagggactggagatcctgacaaagccagtccaataaccactttgac |
| | taacccgatcatcctatgattcccagaatatatctcgtcgaatgatttcagaatg |
| | tgccgcaggatcctgaacgagtaaccattcgggctacacactttaacccttccgt |
| | tgatacaaaagttcctcatgttcttcttgcctgtaagttctttcagcgggacgta |
| | ttcagggggtggaagccacaagtcatcgtcatccagaggggctgacgcgggagag |
| | gatttttgagtgtcctcgtccctgcggtttttcactatcttacgtaggaggtt |
| SEQ ID | ATGAACCTGCTGCGGAAGATCGTGAAGAATCGGCGCGACGAGGACACCCAGAAGT |
| NO: 4023 | CTAGCCCTGCTTCTGCCCCTCTGGACGACGATGATCTGTGGCTGCCTCCTCCAGA |
| M gene | GTACGTGCCCCTGAAAGAGCTGACCGGCAAAAAGAACATGCGGAATTTCTGCATC |
| codon | AACGGCCGCGTGAAAGTGTGCAGCCCCAACGGCTACAGCTTCAGAATCCTGCGGC |
| optimized | ACATCCTGAAGTCCTTCGACGAAATCTACAGCGGCAACCACCGGATGATCGGCCT |
| (nucleic | GGTCAAGGTTGTGATTGGACTGGCCCTGAGCGGCTCTCCTGTTCCTGAAGGCCTG |
| acid) | AACTGGGTCTACAAGCTGCGGCGGACATTCATTTTTCAGTGGGCCGACAGCAGAG |
| | GCCCTCTGGAAGGCGAGGAACTGGAATACTCCCAAGAGATCACCTGGGACGACGA |
| | CACCGAGTTTGTGGGCCTGCAGATCAGAGTGATCGCCAAGCAGTGCCACATCCAG |
| | GGCAGAGTGTGGTGCATCAACATGAACCCCAGAGCCTGCCAGCTTTGGAGCGACA |
| | TGTCTCTGCAGACCCAGCGGAGCGAAGAGGACAAGGATtcctctctgcttctaga |
| | ataa |
| SEQ ID | NLLRKIVKNRRDEDTQKSSPASAPLDDDDLWLPPPEYVPLKELTGKKNMRNFCIN |
| NO: 4006 | GRVKVCSPNGYSFRILRHILKSFDEIYSGNHRMIGLVKVVIGLALSGSPVPEGLN |
| M gene | WVYKLRRTFIFQWADSRGPLEGEELEYSQEITWDDDTEFVGLQIRVIAKQCHIQG |
| (amino | RVWCINMNPRACQLWSDMSLQTQRSEEDKDSSLLLE |
| acid) | |
| SEQ ID | agcaagatctttgtcaatcctagtgctattagagccggtctggccgatcttgaga |
| NO: 4007 | tggctgaagaaactgttgatctgatcaatagaaatatcgaagacaatcaggctca |
| P gene | tctccaaggggaacccatagaggtggacaatctccctgaggatatggggcgactt |
| (nucleic | cacctggatgatggaaaatcgcccaaccatggtgagatagccaaggtgggagaag |
| acid) | gcaagtatcgagaggactttcagatggatgaaggagaggatcctagettcctgtt |
| | ccagtcatacctggaaaatgttggagtccaaatagtcagacaaatgaggtcagga |
| | gagagatttctcaagatatggtcacagaccgtagaagagattatatcctatgtcg |
| | cggtcaactttcccaaccctccaggaaagtettcagaggataaatcaacccagac |
| | tactggccgagagctcaagaaggagacaacacccactccttctcagagagaaagc |
| | caatcatcgaaagccaggatggeggctcaaattgcttctggccctccagcccttg |
| | aatggteggctaccaatgaagaggatgatctatcagtggaggctgagatcgctca |
| | ccagattgcagaaagtttctccaaaaaatataagtttccctctcgatcctcaggg |
| | atactcttgtataattttgagcaattgaaaatgaaccttgatgatatagttaaag |
| | aggcaaaaaatgtaccaggtgtgacccgtttagcccatgacgggtccaaactccc |
| | cctaagatgtgtactgggatgggtcgctttggccaactctaagaaattccagttg |
| | ttagtcgaatccgacaagctgagtaaaatcatgcaagatgacttgaatcgctata |
| | catcttgc |
| SEQ ID | ATGAGCAAGATCTTTGTCAATCCTAGTGCTATCAGAGCCGGCCTGGCTGATCTTG |
| NO: 4015 | AGATGGCCGAGGAAACCGTGGACCTGATCAACCGGAACATCGAGGACAATCAGGC |
| P gene | CCATCTGCAGGGCGAGCCTATCGAGGTTGACAATCTGCCCGAGGACATGGGCAGA |
| codon | CTGCACCTGGATGATGGCAAGAGCCCTAACCACGGCGAGATCGCCAAAGTTGGCG |
| optimized | AGGGCAAGTACCGCGAGGACTTCCAGATGGACGAGGGCGAAGATCCCAGCTTCCT |
| (nucleic | GTTCCAGTCCTACCTGGAAAACGTGGGCGTGCAGATCGTGCGGCAGATGAGAAGC |
| acid) | GGCGAGCGGTTCCTGAAGATTTGGAGCCAGACCGTGGAAGAGATCATCAGCTACG |
| | TGGCCGTGAACTTCCCCAATCCTCCAGGCAAGAGCAGCGAGGACAAGAGCACACA |
| | GACCACCGGCAGAGAGCTGAAGAAAGAGACAACCCCTACACCTAGCCAGAGAGAG |
| | AGCCAGAGCAGCAAGGCCAGAATGGCCGCTCAGATTGCCTCTGGACCTCCAGCTC |
| | TTGAGTGGAGCGCCACCAACGAAGAGGACGACCTGTCTGTGGAAGCCGAGATTGC |
| | CCACCAGATCGCCGAGAGCTTCAGCAAGAAGTACAAGTTCCCCAGCAGAAGCAGC |
| | GGCATCCTGCTGTATAACTTCGAGCAGCTGAAGATGAACCTGGACGACATCGTGA |
| | AAGAGGCCAAGAACGTCCCCGGCGTGACAAGACTGGCCCACGATGGATCTAAGCT |
| | GCCCCTGAGATGTGTGCTCGGATGGGTTGCCCTGGCCAATAGCAAGAAATTCCAG |
| | CTGCTGGTGGAAAGCGACAAGCTGTCCAAGATCATGCAGGATGACTTGAATCGCT |
| | ATACATCTTGCTAA |
| SEQ ID | SKIFVNPSAIRAGLADLEMAEETVDLINRNIEDNQAHLQGEPIEVDNLPEDMGRL |
| NO: 4008 | HLDDGKSPNHGEIAKVGEGKYREDFQMDEGEDPSFLFQSYLENVGVQIVRQMRSG |
| P gene | ERFLKIWSQTVEEIISYVAVNFPNPPGKSSEDKSTQTTGRELKKETTPTPSQRES |
| (amino | QSSKARMAAQIASGPPALEWSATNEEDDLSVEAEIAHQIAESFSKKYKFPSRSSG |
| acid) | ILLYNFEQLKMNLDDIVKEAKNVPGVTRLAHDGSKLPLRCVLGWVALANSKKFQL |
| | LVESDKLSKIMQDDLNRYTSC |
| SEQ ID | atggttcctcaggctctcctgtttgtaccccttctggtttttccattgtgttttg |
| NO: 4009 | ggaaattccctatttacacgataccagacaagcttggtccctggagtccgattga |
| G gene | catacatcacctcagctgcccaaacaatttggtagtggaggacgaaggatgcacc |
| (nucleic | aacctgtcagggttctcctacatggaacttaaagttggatacatcttagccataa |
| acid) | aagtgaacgggttcacttgcacaggcgttgtgacggaggctgaaacctacactaa |
| | cttcgttggttatgtcacaaccacgttcaaaagaaagcatttccgcccaacacca |
| | gatgcatgtagagccgcgtacaactggaagatggccggtgaccccagatatgaag |
| | agtctctacacaatccgtaccctgactaccgctggcttcgaactgtaaaaaccac |
| | caaggagtctctcgttatcatatctccaagtgtggcagatttggacccatatgac |
| | agatcccttcactcgagggtcttccctagcgggaagtgctcaggagtagaggtgt |
| | cttctacctactgctccactaaccacgattacaccatttggatgcccgagaatcc |
| | gagactagggatgtcttgtgacatttttaccaatagtagagggaagagagcatcc |
| | aaagggagtgagacttgcggctttgtagatgaaagaggcctatataagtctttaa |
| | aaggagcatgcaaactcaagttatgtggagttctaggacttagacttatggatgg |
| | aacatgggtctcgatgcaaacatcaaatgaaaccaaatggtgccctcccgataag |
| | ttggtgaacctgcacgactttcgctcagacgaaattgagcaccttgttgtagagg |
| | agttggtcaggaagagagaggagtgtctggatgcactagagtccatcatgacaac |
| | caagtcagtgagtttcagacgtctcagtcatttaagaaaacttgtccctgggttt |
| | ggaaaagcatataccatattcaacaagaccttgatggaagccgatgctcactaca |
| | agtcagtcagaacttggaatgagatcctcccttcaaaagggtgtttaagagttgg |
| | ggggaggtgtcatcctcatgtgaacggggtgtttttcaatggtataatattagga |
| | cctgacggcaatgtcttaatcccagagatgcaatcatccctcctccagcaacata |
| | tggagttgttggaatcctcggttatcccccttgtgcaccccctggcagacccgtc |
| | taccgttttcaaggacggtgacgaggctgaggattttgttgaagttcaccttccc |
| | gatgtgcacaatcaggtctcaggagttgacttgggtctcccgaactgggggaagt |
| | atgtattactgagtgcaggggccctgactgccttgatgttgataattttcctgat |
| | gacatgttgtagaagagtcaatcgatcagaacctacgcaacacaatctcagaggg |
| | acagggagggaggtgtcagtcactccccaaagcgggaagatcatatcttcatggg |
| | aatcacacaagagtgggggtgagaccagactg |
| SEQ ID | ATGGTTCCTCAGGCTCTGCTGTTCGTGCCCCTGCTGGTGTTCCCTCTGTGCTTCG |
| NO: 4016 | GCAAGTTCCCCATCTACACAATCCCCGACAAGCTCGGCCCTTGGAGCCCTATCGA |
| G gene | TATTCACCACCTGAGCTGCCCCAACAACCTGGTGGTGGAAGATGAGGGCTGCACC |
| codon | AACCTGAGCGGCTTCAGCTACATGGAACTGAAAGTGGGCTACATCCTGGCCATCA |
| optimized | AAGTGAACGGCTTCACCTGTACCGGCGTCGTGACAGAGGCCGAGACATACACCAA |
| (nucleic | CTTCGTGGGCTACGTGACCACCACCTTCAAGCGGAAGCACTTCAGACCCACACCT |
| acid) | GACGCCTGTAGAGCCGCCTACAACTGGAAAATGGCCGGCGATCCCAGATACGAGG |
| | AAAGCCTGCACAACCCCTATCCTGACTACAGATGGCTGAGAACCGTGAAAACCAC |
| | CAAAGAGAGCCTGGTCATCATCAGCCCCAGCGTGGCCGATCTGGACCCCTATGAT |
| | AGATCCCTGCACAGCAGAGTGTTCCCCAGCGGCAAATGTTCTGGCGTGGCCGTGT |
| | CTAGCACCTACTGCTCCACCAACCACGACTACACCATCTGGATGCCCGAGAATCC |
| | CAGACTGGGCATGAGCTGCGACATCTTCACCAACAGCCGGGGCAAGAGAGCCAGC |
| | AAGGGCTCTGAGACATGCGGCTTCGTGGACGAGAGGGGCCTGTATAAGTCTCTGA |
| | AGGGCGCCTGCAAGCTGAAGCTGTGCGGAGTTCTGGGCCTGAGACTGATGGATGG |
| | CACCTGGGTGTCCATGCAGACCAGCAACGAGACAAAGTGGTGCCCTCCTGACAAG |
| | CTGGTCAACCTGCACGACTTCAGAAGCGACGAGATCGAGCATCTGGTGGTCGAGG |
| | AACTCGTGCGGAAGAGGGAAGAATGCCTGGACGCCCTGGAATCCATTATGACCAC |
| | CAAGAGCGTGTCCTTCCGGCGGCTGTCTCACCTGAGAAAACTGGTGCCCGGCTTT |
| | GGCAAGGCTTACACAATCTTCAACAAGACCCTGATGGAAGCCGACGCTCACTACA |
| | AGTCTGTGCGGACCTGGAACGAGATCCTGCCTTCCAAGGGCTGCCTGAGAGTTGG |
| | CGGAAGATGTCACCCTCACGTGAACGGCGTGTTCTTCAACGGCATCATCCTGGGA |
| | CCTGACGGCAACGTGCTGATCCCTGAGATGCAGTCTAGCCTGCTCCAGCAACACA |
| | TGGAATTGCTGGAAAGCAGCGTGATCCCTCTGGTGCACCCTCTGGCCGATCCTAG |
| | CACCGTGTTTAAGGATGGCGACGAGGCCGAGGACTTCGTGGAAGTGCATCTGCCC |
| | GACGTGCACAATCAGGTGTCAGGCGTTGACCTGGGCCTGCCTAACTGGGGCAAAT |
| | ACGTGCTGCTTTCTGCCGGCGCTCTGACAGCCCTGATGCTGATCATCTTCCTGAT |
| | GACCTGCTGTCGGAGAGTGAACAGAAGCGAGCCCACACAGCACAACCTGAGAGGC |
| | ACAGGCAGAGAAGTGTCCGTGACACCTCAGAGCGGCAAGATCATCAGCAGCTGGG |
| | AGAGCCACAAGAGCGGCGGAGAAACAAGACTgTAA |
| SEQ ID | MVPQALLFVPLLVFPLCFGKFPIYTIPDKLGPWSPIDIHHLSCPNNLVVEDEGCT |
| NO: 4010 | NLSGFSYMELKVGYILAIKVNGFTCTGVVTEAETYTNFVGYVTTTFKRKHFRPTP |
| G gene | DACRAAYNWKMAGDPRYEESLHNPYPDYRWLRTVKTTKESLVIISPSVADLDPYD |
| (amino | RSLHSRVFPSGKCSGVAVSSTYCSTNHDYTIWMPENPRLGMSCDIFTNSRGKRAS |
| acid) | KGSETCGFVDERGLYKSLKGACKLKLCGVLGLRLMDGTWVSMQTSNETKWCPPDK |
| | LVNLHDFRSDEIEHLVVEELVRKREECLDALESIMTTKSVSFRRLSHLRKLVPGF |
| | GKAYTIFNKTLMEADAHYKSVRTWNEILPSKGCLRVGGRCHPHVNGVFFNGIILG |
| | PDGNVLIPEMQSSLLQQHMELLESSVIPLVHPLADPSTVFKDGDEAEDFVEVHLP |
| | DVHNQVSGVDLGLPNWGKYVLLSAGALTALMLIIFLMTCCRRVNRSEPTQHNLRG |
| | TGREVSVTPQSGKIISSWESHKSGGETRL |

In certain embodiments, the recombinant rabies virus genome comprises an N gene having a nucleic acid sequence that is about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the nucleic acid sequence set forth in SEQ ID NO: 4001. In certain embodiments, the recombinant rabies virus genome comprises an N gene having a nucleic acid sequence that is at least 60%, at least 65%, about 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the nucleic acid sequence set forth in SEQ ID NO: 4001. In certain embodiments, the recombinant rabies virus genome comprises an N gene comprising the nucleic acid sequence set forth in SEQ ID NO: 4001. In certain embodiments, the recombinant rabies virus genome comprises an N gene consisting of the nucleic acid sequence set forth in SEQ ID NO: 4001. In certain embodiments, the N gene encodes for an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the amino acid sequence set forth in SEQ ID NO: 4002. In certain embodiments, the N gene encodes for an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the amino acid sequence set forth in SEQ ID NO: 4002. In certain embodiments, the N gene encodes for an amino acid sequence comprising the amino acid sequence set forth in SEQ ID NO: 4002. In certain embodiments, the N gene encodes for an amino acid sequence consisting of the amino acid sequence set forth in SEQ ID NO: 4002.

In certain embodiments, the recombinant rabies virus genome comprises an L gene having a nucleic acid sequence that is about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the nucleic acid sequence set forth in SEQ ID NO: 4003. In certain embodiments, the recombinant rabies virus genome comprises an L gene having a nucleic acid sequence that is at least 60%, at least 65%, about 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the nucleic acid sequence set forth in SEQ ID NO: 4003. In certain embodiments, the recombinant rabies virus genome comprises an L gene comprising the nucleic acid sequence set forth in SEQ ID NO: 4003. In certain embodiments, the recombinant rabies virus genome comprises an L gene consisting of the nucleic acid sequence set forth in SEQ ID NO: 4003. In certain embodiments, the L gene encodes for an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the amino acid sequence set forth in SEQ ID NO: 4004. In certain embodiments, the L gene encodes for an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the amino acid sequence set forth in SEQ ID NO: 4004. In certain embodiments, the L gene encodes for an amino acid sequence comprising the amino acid sequence set forth in SEQ ID NO: 4004. In certain embodiments, the L gene encodes for an amino acid sequence consisting of the amino acid sequence set forth in SEQ ID NO: 4004.

In certain embodiments, the recombinant rabies virus genome comprises an M gene having a nucleic acid sequence that is about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the nucleic acid sequence set forth in SEQ ID NO: 4005. In certain embodiments, the recombinant rabies virus genome comprises an M gene having a nucleic acid sequence that is at least 60%, at least 65%, about 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the nucleic acid sequence set forth in SEQ ID NO: 4005. In certain embodiments, the recombinant rabies virus genome comprises an M gene comprising the nucleic acid sequence set forth in SEQ ID NO: 4005. In certain embodiments, the recombinant rabies virus genome comprises an M gene consisting of the nucleic acid sequence set forth in SEQ ID NO: 4005. In certain embodiments, the M gene encodes for an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the amino acid sequence set forth in SEQ ID NO: 4006. In certain embodiments, the M gene encodes for an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the amino acid sequence set forth in SEQ ID NO: 4006. In certain embodiments, the M gene encodes for an amino acid sequence comprising the amino acid sequence set forth in SEQ ID NO: 4006. In certain embodiments, the M gene encodes for an amino acid sequence consisting of the amino acid sequence set forth in SEQ ID NO: 4006.

In certain embodiments, the recombinant rabies virus genome comprises a P gene having a nucleic acid sequence that is about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the nucleic acid sequence set forth in SEQ ID NO: 4007. In certain embodiments, the recombinant rabies virus genome comprises a P gene having a nucleic acid sequence that is at least 60%, at least 65%, about 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the nucleic acid sequence set forth in SEQ ID NO: 4007. In certain embodiments, the recombinant rabies virus genome comprises a P gene comprising the nucleic acid sequence set forth in SEQ ID NO: 4007. In certain embodiments, the recombinant rabies virus genome comprises a P gene consisting of the nucleic acid sequence set forth in SEQ ID NO: 4007. In certain embodiments, the P gene encodes for an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the amino acid sequence set forth in SEQ ID NO: 4008. In certain embodiments, the P gene encodes for an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the amino acid sequence set forth in SEQ ID NO: 4008. In certain embodiments, the P gene encodes for an amino acid sequence comprising the amino acid sequence set forth in SEQ ID NO: 4008. In certain embodiments, the P gene encodes for an amino acid sequence consisting of the amino acid sequence set forth in SEQ ID NO: 4008.

In certain embodiments, the recombinant rabies virus genome comprises a G gene having a nucleic acid sequence that is about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the nucleic acid sequence set forth in SEQ ID NO: 4009. In certain embodiments, the recombinant rabies virus genome comprises a G gene having a nucleic acid sequence that is at least 60%, at least 65%, about 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the nucleic acid sequence set forth in SEQ ID NO: 4009. In certain embodiments, the recombinant rabies virus genome comprises a G gene comprising the nucleic acid sequence set forth in SEQ ID NO: 4009. In certain embodiments, the recombinant rabies virus genome comprises a G gene consisting of the nucleic acid sequence set forth in SEQ ID NO: 4009. In certain embodiments, the G gene encodes for an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% identical to the amino acid sequence set forth in SEQ ID NO: 4010. In certain embodiments, the G gene encodes for an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to the amino acid sequence set forth in SEQ ID NO: 4010. In certain embodiments, the G gene encodes for an amino acid sequence comprising the amino acid sequence set forth in SEQ ID NO: 4010. In certain embodiments, the G gene encodes for an amino acid sequence consisting of the amino acid sequence set forth in SEQ ID NO: 4010.

Each of the genes comprised within a recombinant rabies virus genome of the present disclosure may be operably linked to a transcriptional regulatory element. In certain embodiments, wherein the genes are linked on a single expression cassette, a single transcriptional regulatory element may be capable of controlling the expression of the genes. In certain embodiments, each gene is operably linked to a separate transcriptional regulatory element. In certain embodiments, the transcriptional regulatory elements for each gene may be the same. In certain embodiments, the transcriptional regulatory elements for each gene may be different.

In certain embodiments, each of the genes are operably linked to a transcriptional regulatory element, wherein the transcriptional regulatory element is capable of controlling the expression of the gene that is operably linked thereto. In certain embodiments, the transcriptional regulatory element comprises a transcription initiation signal. The transcription initiation signal can be endogenous or exogenous to the rabies virus. In certain embodiments, the transcription initiation signal is a synthetic transcription initiation signal. In certain embodiments, the nucleic acid encoding a transgene is further operably linked to a transcription termination polyadenylation signal. The transcription termination polyadenylation signal can be endogenous or exogenous to the rabies virus. In certain embodiments, the transcription termination polyadenylation signal is a synthetic transcription termination polyadenylation signal. Examples of suitable transcription initiation signals and transcriptional termination polyadenylaton signals are known to those of ordinary skill in the art, and are described in, e.g., Albertini et al., Adv. Virus. Res. (2011) 79: 1-22; Ogino and Green, Viruses (2019) 11(6): 504; Ogino et al., Nucl. Acids. Res. (2019) 47(1): 299-309; and Ogino and Green, Front. Microbiol. (2019) 10: 1490, the disclosures of which are herein incorporated by reference in their entireties.

In certain embodiments, the recombinant rabies virus is replication incompetent. As used herein, the term “replication incompetent” refers to a virus that is incapable of completing a full replication cycle in a host cell after infection. In general, viruses may be rendered replication incompetent through the deletion or inactivation of viral genes necessary for viral replication. In certain embodiments, the recombinant rabies virus is rendered replication incompetent through the deletion or inactivation of any one or more of the P gene, the L gene, the M gene, the N gene, and the G gene, in the recombinant rabies virus genome. In other embodiments, the recombinant rabies virus is replication deficient. As used herein, the term “replication deficient” refers to a modified virus that is capable of completing a full replication cycle in a host cell after infection less efficiently compared to a wild-type or unmodified virus.

### C. GUIDE RNA & RECOMBINANT RECOMBINANT RABIES VIRUS GENOMES ENCODING THE SAME

In one aspect, the disclosure provides a recombinant rabies virus genome, comprising a nucleic acid encoding a first guide RNA (gRNA) that comprises a 5′ end and a 3′ end; and a nucleic acid encoding a first transfer RNA (tRNA) positioned at one of both of upstream of the 5′ end of the nucleic acid encoding the first gRNA or downstream of the 3′ end of the nucleic acid encoding the first gRNA.

In certain embodiments, the recombinant rabies virus genome comprises a nucleic acid encoding a second tRNA. In certain embodiments, the recombinant rabies virus genome comprises a nucleic acid encoding a third tRNA. In certain embodiments, the recombinant rabies virus genome comprises a nucleic acid encoding a fourth tRNA. In certain embodiments, the recombinant rabies virus genome comprises a nucleic acid encoding a fifth tRNA.

In certain embodiments, the nucleic acid encoding the first tRNA is positioned upstream of the 5′ end of the nucleic acid encoding the first gRNA; and the nucleic acid encoding the second tRNA is positioned downstream of the 3′ end of the nucleic acid encoding the first gRNA.

In certain embodiments, the nucleotide sequence of the first tRNA and the nucleotide sequence of the second tRNA, third tRNA, fourth tRNA, and/or fifth tRNA are at least 90%, 91%, 92%, 93%, 94%, 95%, 96%, 97%, 98%, 99%, or 100% identical.

In certain embodiments, the first tRNA and the second tRNA, third tRNA, fourth tRNA, and/or fifth tRNA specify the same amino acid. For example, the first tRNA and the second tRNA possess different anti-codon loop sequences, each anti-codon loop sequence corresponding to the same amino acid (e.g., a first tRNA with an anti-codon loop sequence comprising 5′ GGC 3′ specifying Ala, and a second tRNA with an anti-codon loop sequence comprising 5′ AGC 3′, also specifying Ala).

In certain embodiments, the first tRNA and the second tRNA, third tRNA, fourth tRNA, and/or fifth tRNA specify different amino acids. For example, the first tRNA and the second tRNA possess different anti-codon loop sequences, each anti-codon loop sequence corresponding to different amino acids (e.g., a first tRNA with an anti-codon loop sequence comprising 5′ GGC 3′ specifying Ala, and a second tRNA with an anti-codon loop sequence comprising 5′ AAA 3′, specifying Phe).

In certain embodiments, the recombinant rabies virus genome comprises two or more nucleic acids encoding the first tRNA, second tRNA, third tRNA, fourth tRNA, and/or fifth tRNA. In certain embodiments, the recombinant rabies virus genome comprises two nucleic acids encoding the first tRNA, second tRNA, third tRNA, fourth tRNA, and/or fifth tRNA. In certain embodiments, the recombinant rabies virus genome comprises three nucleic acids encoding the first tRNA, second tRNA, third tRNA, fourth tRNA, and/or fifth tRNA. In certain embodiments, the recombinant rabies virus genome comprises four nucleic acids encoding the first tRNA, second tRNA, third tRNA, fourth tRNA, and/or fifth tRNA. In certain embodiments, the recombinant rabies virus genome comprises five nucleic acids encoding the first tRNA, second tRNA, third tRNA, fourth tRNA, and/or fifth tRNA.

In certain embodiments, the recombinant rabies virus genome comprises a nucleic acid encoding a second gRNA, a third gRNA, a fourth gRNA, and/or a fifth gRNA.

In certain embodiments, the two or more nucleic acids encode identical gRNA. In certain embodiments, the two or more nucleic acids encode at least one different gRNA. In certain embodiments, the nucleotide sequence of the first gRNA and the nucleotide sequence of the second gRNA, a third gRNA, a fourth gRNA, and/or a fifth gRNA are at least 90%, 91%, 92%, 93%, 94%, 95%, 96%, 97%, 98%, 99%, or 100% identical.

In certain embodiments, the first gRNA and the second gRNA, a third gRNA, a fourth gRNA, and/or a fifth gRNA specifically hybridize to the same target nucleic acid sequence. In certain embodiments, the first gRNA and the second gRNA, a third gRNA, a fourth gRNA, and/or a fifth gRNA specifically hybridize to different target nucleic acid sequence.

In certain embodiments, the first tRNA, second tRNA, third tRNA, fourth tRNA, and/or fifth tRNA is each selected from the group consisting of: tRNA-ala, tRNA-arg, tRNA-asn, tRNA-asp, tRNA-cys, tRNA-gln, tRNA-gly, tRNA-his, tRNA-ile, tRNA-leu, tRNA-lys, tRNA-met, tRNA-phe, tRNA-pro, tRNA-pyl, tRNA-sec, tRNA-ser, tRNA-thr, tRNA-trp, tRNA-tyr, and tRNA-val.

In certain embodiments, the recombinant rabies virus genome comprises a nucleic acid encoding a negative-strand RNA virus gene.

In certain embodiments, the recombinant rabies virus genome comprises a nucleic acid encoding a transgene (e.g., a nucleobase editor).

In certain embodiments, the nucleic acid encoding the first gRNA and the nucleic acid encoding the first tRNA are positioned between two nucleic acids each encoding a negative-strand RNA virus gene.

In certain embodiments, the nucleic acid encoding the first gRNA and the nucleic acid encoding the first tRNA are positioned between two nucleic acids each encoding a transgene.

In certain embodiments, the nucleic acid encoding the first gRNA and the nucleic acid encoding the first tRNA are positioned between a nucleic acid encoding a negative-strand RNA virus gene and a nucleic acid encoding a transgene.

In certain embodiments, the recombinant rabies virus genome comprises a gRNA expression cassette comprising, from 5′ to 3′, a negative-strand RNA virus transcription initiation signal, a nucleic acid encoding a tRNA, a nucleic acid encoding a gRNA, and a transcription termination polyadenylation signal.

In certain embodiments, the recombinant rabies virus genome comprises a gRNA expression cassette comprising, from 5′ to 3′, a negative-strand RNA virus transcription initiation signal, a nucleic acid encoding the first tRNA, a nucleic acid encoding the first gRNA, a nucleic acid encoding a second tRNA, and a transcription termination polyadenylation signal.

In certain embodiments, the recombinant rabies virus genome comprises a gRNA expression cassette comprising, from 5′ to 3′, a negative-strand RNA virus transcription initiation signal, a nucleic acid encoding the first tRNA, a nucleic acid encoding the first gRNA, a nucleic acid encoding a second tRNA, a nucleic acid encoding a second gRNA, and a transcription termination polyadenylation signal.

In certain embodiments, the recombinant rabies virus genome comprises a gRNA expression cassette comprising, from 5′ to 3′, a negative-strand RNA virus transcription initiation signal, a nucleic acid encoding the first tRNA, a nucleic acid encoding the first gRNA, a nucleic acid encoding a second tRNA, a nucleic acid encoding a second gRNA, and a transcription termination polyadenylation signal.

In certain embodiments, the recombinant rabies virus genome comprises a gRNA expression cassette comprising, from 5′ to 3′, a negative-strand RNA virus transcription initiation signal, a nucleic acid encoding the first tRNA, a nucleic acid encoding the first gRNA, a nucleic acid encoding a second tRNA, a nucleic acid encoding a second gRNA, a nucleic acid encoding a third tRNA, and a transcription termination polyadenylation signal.

In certain embodiments of the gRNA expression cassette, the nucleic acid encoding the first tRNA, second tRNA, and/or third tRNA are identical. In certain embodiments of the gRNA expression cassette, the nucleic acid encoding the first tRNA, second tRNA, and/or third tRNA are different. In certain embodiments of the gRNA expression cassette, the nucleotide sequence of the first tRNA and the nucleotide sequence of the second tRNA are at least 90%, 91%, 92%, 93%, 94%, 95%, 96%, 97%, 98%, 99%, or 100% identical. In certain embodiments of the gRNA expression cassette, the first tRNA and the second tRNA specify the same amino acid. In certain embodiments of the gRNA expression cassette, the first tRNA and the second tRNA specify different amino acids. In certain embodiments of the gRNA expression cassette, the nucleic acid encoding the first gRNA and/or second gRNA are identical. In certain embodiments of the gRNA expression cassette, the nucleic acid encoding the first gRNA and/or second gRNA are different. In certain embodiments of the gRNA expression cassette, the nucleotide sequence of the first gRNA and the nucleotide sequence of the second gRNA are at least 90%, 91%, 92%, 93%, 94%, 95%, 96%, 97%, 98%, 99%, or 100% identical. In certain embodiments of the gRNA expression cassette, the first gRNA and the second gRNA specifically hybridize to the same target nucleic acid sequence. In certain embodiments of the gRNA expression cassette, the first gRNA and the second gRNA specifically hybridize to different target nucleic acid sequence. In certain embodiments of the gRNA expression cassette, the transcription termination polyadenylation signal comprises an endogenous transcription termination polyadenylation signal. In certain embodiments of the gRNA expression cassette, the transcription termination polyadenylation signal comprises a heterologous transcription termination polyadenylation signal.

### D. THERAPEUTIC TRANSGENES

In certain embodiments, a recombinant rabies virus genome of the present disclosure encodes a nucleic acid comprising a therapeutic transgene. As used herein, the term “therapeutic” refers to treatment and/or prophylaxis. As used herein, the term “therapeutic transgene” refers to a transgene that encodes a transgene product that is capable of effecting treatment and/or prophylaxis to a subject in need. In certain embodiments, the therapeutic effect is accomplished by suppression, remission, or eradication of a disease state suffered by the subject. The therapeutic transgene may encode any therapeutic agent that is capable of effecting treatment and/or prophylaxis in a subject in need, resulting in suppression, remission, or eradication of a disease state in the subject. In certain embodiments, the therapeutic transgene encodes a precursor of a transgene product that is capable of effecting treatment and/or prophylaxis to a subject in need thereof once processed, e.g., processed in a cell.

In certain embodiments, the nucleic acid encoding the therapeutic transgene is greater than: about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, about 2,000 bp, about 2,100 bp, about 2,200 bp, about 2,300 bp, about 2,400 bp, about 2,500 bp, about 2,600 bp, about 2,700 bp, about 2,800 bp, about 2,900 bp, or about 3,000 bp.

In certain embodiments, the nucleic acid encoding the therapeutic transgene is greater than about 300 bp (e.g., the therapeutic transgene is about 350 bp, about 400 bp, about 450 bp, about 500 bp, about 550 bp, about 600 bp, or about 650 bp). In certain embodiments, the nucleic acid encoding the therapeutic transgene is greater than about 650 bp (e.g., the therapeutic transgene is about 700 bp, about 750 bp, about 800 bp, about 850 bp, about 900 bp, about 950 bp, or about 1,000 bp). In certain embodiments, the nucleic acid encoding the therapeutic transgene is greater than about 1,000 bp (e.g., the therapeutic transgene is about 1,500 bp, about 2,000 bp, about 2,500 bp, or about 3,000 bp). In certain embodiments, the nucleic acid encoding the therapeutic transgene is greater than about 3,000 bp (e.g., the therapeutic transgene is about 3,500 bp, about 4,000 bp, or about 4,500 bp).

In certain embodiments, the nucleic acid encoding the therapeutic transgene is greater than about 4,500 bp (e.g., the therapeutic transgene is about 5,000 bp, about 5,500 bp, about 6,000 bp, about 6,500 bp, about 7,000 bp, about 7,500 bp, about 8,000 bp, or about 8,500 bp).

In certain embodiments, the nucleic acid encoding the therapeutic transgene is greater than about 8,500 bp (e.g., the therapeutic transgene is about 9,000 bp, about 9,500 bp, or about 10,000 bp).

In certain embodiments, the nucleic acid encoding the therapeutic transgene is greater than about 10,000 bp (e.g., the therapeutic transgene is about 10,500 bp, about 11,000 bp, about 11,500 bp, about 12,000 bp, about 12,500 bp, about 13,000 bp, about 13,500 bp, about 14,000 bp, about 14,500 bp, or about 15,000 bp).

In certain embodiments, the nucleic acid encoding the therapeutic transgene is between about 4,000 bp and about 6,000 bp (e.g., the therapeutic transgene is about 4,000 bp, about 4,500 bp, about 5,000 bp, about 5,500 bp, or about 6,000 bp).

In certain embodiments, the therapeutic transgene encodes a therapeutic nucleic acid. The therapeutic transgene may encode any therapeutic nucleic acid known in the art, for example, without limitation, any antisense RNA (single-stranded RNA), any small interfering RNA (double-stranded RNA), any RNA aptamer, and/or any messenger RNA (mRNA). For example, the therapeutic transgene can encode, without limitation, a miRNA, a miRNA mimic, a siRNA, a shRNA, a gRNA, a long noncoding RNA, an enhancer RNA, a RNA aptazyme, a RNA aptamer, an antagomiR, and/or a synthetic RNA. In certain embodiments, a therapeutic nucleic acid may be a RNA binding site, e.g., a miRNA binding site. Various other types of therapeutic nucleic acids are known to those of ordinary skill in the art.

In certain embodiments, the therapeutic transgene encodes a therapeutic polypeptide. The therapeutic transgene may encode any therapeutic polypeptide known in the art, for example, without limitation, a therapeutic polypeptide that can replace a deficient or abnormal protein; a therapeutic polypeptide that can augment an existing pathway; a therapeutic polypeptide that can provide a novel function or activity (e.g., a novel function or activity beneficial to a subject suffering from the lack thereof); a therapeutic polypeptide that interferes with a molecule or an organism (e.g., an organism that is different to the organism that hosts the target cell); and/or a therapeutic polypeptide that delivers other compounds or proteins (e.g., a radionuclide, a cytotoxic drug, and/or an effector protein). For example, the therapeutic transgene can encode, without limitation, a nucleic acid modifying protein (e.g., an adenine or cytidine base editor) or system, an antibody or antibody-based drug, an anticoagulant, a blood factor, a bone morphogenetic protein, an engineered protein scaffold, an enzyme, an Fc fusion protein, a growth factor, a hormone, an interferon, an interleukin, and/or a thrombolytic. Various other types of therapeutic polypeptides are known to those of ordinary skill in the art.

In certain embodiments, the therapeutic transgene encodes a nucleic acid editing system or components thereof. In some embodiments the therapeutic transgene encodes a protein comprising a nucleic acid binding protein (e.g., a zinc finger, a TALE, or a nucleic acid programmable nucleic acid binding protein, such as Cas9). In some embodiments, the nucleic acid editing system component is a nucleic acid programmable nucleic acid binding protein (e.g., Cas9). In some embodiments, the nucleic acid editing system component is a guide RNA (gRNA).

In some embodiments, the therapeutic transgene encodes a CRISPR system. In some embodiments, the CRISPR system comprises a nucleobase editor comprising a polynucleotide programmable nucleotide binding domain and a nucleobase editing domain. In some embodiments, the nucleobase editing domain is an adenosine deaminase, cytidine deaminase, cytosine deaminase, or a functional variant thereof (e.g, a functional variant capable of deaminating a nucleobase in a nucleic acid molecule such as DNA or RNA). In some embodiments, the nucleobase editing domain is an adenosine deaminase. In some embodiments, the adenosine deaminase is ABE7.10. In some embodiments, the polynucleotide programmable nucleotide binding domain is a Cas9 polypeptide, a Cas12 polypeptide, or a functional variant thereof. In some embodiments, the CRISPR system further comprises a guide RNA (gRNA) or a nucleic acid encoding a gRNA.

In some embodiments the therapeutic transgene encodes a nucleobase modifying protein (e.g., a base editor protein). In some embodiments the therapeutic transgene encodes an adenosine base editor (e.g., ABE7.10). In some embodiments the therapeutic transgene encodes a cytidine base editor. In some embodiments the therapeutic transgene encodes a cytosine base editor capable of deaminating a cytosine in DNA or RNA.

In certain embodiments, the therapeutic transgene encodes a nucleic acid editing system, e.g., a base editor system further described herein.

It will be readily apparent to those of ordinary skill in the art that a recombinant rabies virus genome of the present disclosure described herein encodes a nucleic acid comprising a therapeutic transgene, wherein the therapeutic transgene encodes a therapeutic polypeptide and/or a therapeutic nucleic acid, e.g., in certain embodiments, the therapeutic transgene encodes a combination of the therapeutic polypeptide and the therapeutic nucleic acid. In certain embodiments, the therapeutic transgene encodes one or more therapeutic polypeptides. In certain embodiments, the therapeutic transgene encodes one or more therapeutic nucleic acids. In certain embodiments, the therapeutic transgene encodes a combination of one or more therapeutic polypeptides and one or more therapeutic nucleic acids. Delivery of a combination of a therapeutic polypeptide and therapeutic nucleic acid into a target cell may serve various purposes known to those of ordinary skill in the art. In certain embodiments, a therapeutic polypeptide may be delivered to a target cell, wherein the delivery is detargeted to certain other cell types. For example, a therapeutic transgene can encode a therapeutic polypeptide and/or therapeutic nucleic acid, and also comprise a miRNA binding site. The miRNA binding site may function for cell type detargeting. For example, miRNA122a, which is expressed exlusively in liver, can be employed for hepatocyte detargeting. See, e.g., Dhungel et al., Molecules (2018) 23(7): 1500.

In certain embodiments, the therapeutic transgene further encodes one or more reporter sequences. Reporter sequences when expressed in the target cell, produces a directly or an indirectly detectable signal. Examples of suitable reporter sequences include, without limitation, sequences encoding for fluorescent proteins (e.g., GFP, RFP, YFP), alkaline phosphatase, thymidine kinase, chloramphenicol acetyltransferase (CAT), luciferase, β-galactosidase (LacZ), and β-lactamase. Sequences encoding for cell surface membrane-bound proteins may also be suitable as reporter sequences, for example, membrane-bound proteins to which high affinity antibodies bind, e.g., influenza hemagglutinin protein (HA), CD2, CD4, CD8, and others known to those of ordinary skill in the art, including, e.g., membrane-bound proteins tagged with an antigen domain (e.g., an HA tag, a FLAG tag, a Myc tag, a polyhistidine tag).

In certain embodiments, the therapeutic transgene does not encode a reporter gene and/or a selectable marker. In certain embodiments, the therapeutic transgene does not encode a fluorescent reporter protein (e.g., GFP, YFP, RFP, tdTomato). In certain embodiments, the therapeutic transgene does not encode β-galactosidase (LacZ). In certain embodiments, the therapeutic transgene does not encode chloramphenicol acetyltransferase (CAT).

In certain embodiments, the therapeutic transgene does not encode a polymerase (e.g., DNA polymerase, DNA-directed RNA polymerase, RNA-directed DNA polumerase (RT), telomerase).

In certain embodiments, the therapeutic transgene does not encode a site-specific recombinase (e.g., Cre, FLP, Hin, or Tre recombinases).

In certain embodiments, the therapeutic transgene does not encode a viral antigen.

In certain embodiments, the therapeutic transgene does not encode a pro-apoptotic protein (e.g., cytochrome c).

In certain embodiments, the therapeutic transgene does not encode an an immunoglobulin (e.g., an immunoglobulin heavy and/or light chain.

In certain embodiments, the therapeutic transgene does not encode a neurotransmitter, a neuropeptide, a receptor, a neuronal growth factor, or a neurome gene.

In certain embodiments, the therapeutic transgene encodes for a therapeutic polypeptide and/or a therapeutic nucleic acid, wherein the therapeutic polypeptide and/or the therapeutic nucleic acid are secreted (e.g., secreted from a cell). For example, a recombinant rabies virus genome of the present disclosure described herein may be introduced into a target cell, wherein the recombinant rabies virus genome encodes a nucleic acid comprising a therapeutic transgene, and wherein the therapeutic transgene encodes a therapeutic polypeptide and/or a therapeutic nucleic acid that is secreted (e.g., a secretable therapeutic transgene and/or a secretable therapeutic nucleic acid). The therapeutic polypeptide and/or nucleic acid upon expression, may be secreted outside of the target cell. In certain embodiments, the therapeutic polypeptide and/or nucleic acid, upon expression, is secreted by virtue of endogenous elements that reside on the therapeutic polypeptide and/or nucleic acid (e.g., an endogenous signal peptide that directs extracellular secretion). In certain embodiments, the therapeutic polypeptide and/or nucleic acid, upon expression, is secreted by virtue of exogenous elements that reside on the therapeutic polypeptide and/or nucleic acid (e.g., an exogenous signal peptide that directs extracellular secretion). Delivery of secretable therapeutic polypeptides and/or nucleic acids are useful in the treatment of certain diseases. For example, lysosomal storage disorders (LSD) that result from the metabolic dysfunction of the lysosome comprise a unique cross-correction characteristic that allows specific extracellular LSD enzymes to be taken up and targeted to the lysosomes of enzyme-deficient or enzyme-abnormal cells. Cross-correction chracteristics of certain enzymes form the basis of approved therapies known as enzyme replacement therapies. See, e.g., Rastall and Amalfitano, Appl. Clin. Genet. (2015) 8: 157-169.

In certain embodiments, a recombinant rabies virus genome of the present disclosure comprises a transcriptional regulatory element operably linked to the nucleic acid encoding a transgene. The transcriptional regulatory element is capable of controlling the expression of the transgene (e.g., expression of the encoded therapeutic polypeptide and/or nucleic acid) that is operably linked thereto. In certain embodiments, the transcriptional regulatory element comprises a transcription initiation signal. The transcription initiation signal can be endogenous or exogenous to the rabies virus. In certain embodiments, the transcription initiation signal is a synthetic transcription initiation signal. In certain embodiments, the nucleic acid encoding a transgene is further operably linked to a transcription termination polyadenylation signal. The transcription termination polyadenylation signal can be endogenous or exogenous to the rabies virus. In certain embodiments, the transcription termination polyadenylation signal is a synthetic transcription termination polyadenylation signal. Examples of suitable transcription initiation signals and transcriptional termination polyadenylaton signals are known to those of ordinary skill in the art, and are described in, e.g., Albertini et al., Adv. Virus. Res. (2011) 79: 1-22; Ogino and Green, Viruses (2019) 11(6): 504; and Ogino and Green, Front. Microbiol. (2019) 10: 1490, the disclosures of which are herein incorporated by reference in their entireties.

A recombinant rabies virus genome of the present disclosure comprising a nucleic acid comprising a therapeutic transgene may further comprise any elements known to those of ordinary skill in the art that aid and/or enhance in the expression of the therapeutic transgene.

Recombinant rabies virus genomes of the present disclosure are incorporated into a recombinant rabies virus particle by methods described herein. In certain embodiments, a recombinant rabies virus particle of the present disclosure comprises a rabies virus glycoprotein and a recombinant rabies virus genome comprising a nucleic acid comprising a therapeutic transgene as described herein. In certain embodiments, the recombinant rabies virus particle comprises: a rabies virus glycoprotein; and a recombinant rabies virus genome comprising a nucleic acid comprising a therapeutic transgene, wherein the genome lacks an endogenous G gene encoding for a rabies virus glycoprotein. In certain embodiments, the recombinant rabies virus particle comprises: a rabies virus glycoprotein; and a recombinant rabies virus genome comprising a nucleic acid comprising a therapeutic transgene, wherein the genome lacks an endogenous G gene encoding for a rabies virus glycoprotein; and wherein the genome lacks an endogenous L gene encoding for a rabies virus polymerase.

### E. NUCLEOBASE EDITORS

In certain exemplary embodiments, therapeutic transgenes useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase or cytidine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain can comprise an endonuclease or an exonuclease. An endonuclease can cleave a single strand of a double-stranded nucleic acid or both strands of a double-stranded nucleic acid molecule. In some embodiments, a nuclease domain of a polynucleotide programmable nucleotide binding domain can cut zero, one, or two strands of a target polynucleotide.

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some embodiments, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein.” Accordingly, disclosed herein is a base editor comprising a polynucleotide programmable nucleotide binding domain comprising all or a portion of a CRISPR protein (i.e. a base editor comprising as a domain all or a portion of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 258), Cas12c/C2c3, Cas12d/CasY, Cas12e/CasX, Cas12g, Cas12h, Cas12i, and Cas12j/Casϕ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Cas9 nuclease sequences and structures are well known to those of skill in the art (See, e.g., “Complete genome sequence of an MI strain of Streptococcus pyogenes.” Ferretti et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607(2011); and “A programmable dual-RNA-guided DNA endonuclease in adaptive bacterial immunity.” Jinek M., et al., Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

High Fidelity Cas9 Domains

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 1423. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA have less off-target effects. In some embodiments, the Cas9 domain (e.g., a wild type Cas9 domain (SEQ ID NOs: 223 and 233)) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a D10A, N497X, a R661X, a Q695X, and/or a Q926X mutation, or a corresponding mutation in any of the amino acid sequences provided herein, wherein X is any amino acid. In some embodiments, the high fidelity Cas9 enzyme is SpCas9(K855A), eSpCas9(1.1), SpCas9-HF1, or hyper accurate Cas9 variant (HypaCas9). In some embodiments, the modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

Cas9 Domains with Reduced Exclusivity

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Exemplary polypeptide sequences for spCas9 proteins capable of binding a PAM sequence are provided in the Sequenc Listing as SEQ ID NOs: 223, 234, and 1304-1307. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

Nickases

In some embodiments, the polynucleotide programmable nucleotide binding domain can comprise a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such embodiments, the residue H840 retains catalytic activity and can thereby cleave a single strand of the nucleic acid duplex. In another example, a Cas9-derived nickase domain can comprise an H840A mutation, while the amino acid residue at position 10 remains a D. In some embodiments, a nickase can be derived from a fully catalytically active (e.g., natural) form of a polynucleotide programmable nucleotide binding domain by removing all or a portion of a nuclease domain that is not required for the nickase activity. For example, where a polynucleotide programmable nucleotide binding domain comprises a nickase domain derived from Cas9, the Cas9-derived nickase domain can comprise a deletion of all or a portion of the RuvC domain or the HNH domain.

In some embodiments, wild-type Cas9 corresponds to, or comprises the following amino acid sequence:

(SEQ ID NO: 223)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMlKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEWKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGELRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD
(single underline: HNH domain;
double underline: RuvC domain).

In some embodiments, the strand of a nucleic acid duplex target polynucleotide sequence that is cleaved by a base editor comprising a nickase domain (e.g., Cas9-derived nickase domain, Cas12-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain, Cas12-derived nickase domain) can cleave the strand of a DNA molecule which is being targeted for editing. In such embodiments, the non-targeted strand is not cleaved.

In some embodiments, a Cas9 nuclease has an inactive (e.g., an inactivated) DNA cleavage domain, that is, the Cas9 is a nickase, referred to as an “nCas9” protein (for “nickase” Cas9). The Cas9 nickase may be a Cas9 protein that is capable of cleaving only one strand of a duplexed nucleic acid molecule (e.g., a duplexed DNA molecule). In some embodiments the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

The amino acid sequence of an exemplary catalytically Cas9 nickase (nCas9) is as follows:

(SEQ ID NO: 234)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFiKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some embodiments, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some embodiments, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most embodiments, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag.

In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

### Catalyically Dead Nucleases

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

Additional suitable nuclease-inactive dCas9 domains will be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

In some embodiments, dCas9 corresponds to, or comprises in part or in whole, a Cas9 amino acid sequence having one or more mutations that inactivate the Cas9 nuclease activity. In some embodiments, the nuclease-inactive dCas9 domain comprises a D10X mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. In some embodiments, a nuclease-inactive Cas9 domain comprises the amino acid sequence set forth in Cloning vector pPlatTET-gRNA2 (Accession No. BAV54124).

In some embodiments, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some embodiments, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

As another non-limiting example, in some embodiments, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some embodiments, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some embodiments, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such embodiments, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises a N579A mutation, or a corresponding mutation in any of the amino acid sequences provided in the Sequence Listing submitted herewith.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRV PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

In some embodiments, one of the Cas9 domains present in the fusion protein may be replaced with a guide nucleotide sequence-programmable DNA-binding protein domain that has no requirements for a PAM sequence. In some embodiments, the Cas9 is an SaCas9. Residue A579 of SaCas9 can be mutated from N579 to yield a SaCas9 nickase. Residues K781, K967, and H1014 can be mutated from E781, N967, and R1014 to yield a SaKKH Cas9.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella 1 (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9.

Furthermore, Cpf1, unlike Cas9, does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins that are more similar to types I and III than type II systems. Functional Cpf1 does not require the trans-activating CRISPR RNA (tracrRNA), therefore, only CRISPR (crRNA) is required. This benefits genome editing because Cpf1 is not only smaller than Cas9, but also it has a smaller sgRNA molecule (approximately half as many nucleotides as Cas9). The Cpf1-crRNA complex cleaves target DNA or RNA by identification of a protospacer adjacent motif 5′-YTN-3′ or 5′-TTN-3′ in contrast to the G-rich PAM targeted by Cas9. After identification of PAM, Cpf1 introduces a sticky-end-like DNA double-stranded break having an overhang of 4 or 5 nucleotides.

In some embodiments, the Cas9 is a Cas9 variant having specificity for an altered PAM sequence. In some embodiments, the Additional Cas9 variants and PAM sequences are described in Miller, S. M., et al. Continuous evolution of SpCas9 variants compatible with non-G PAMs, Nat. Biotechnol. (2020), the entirety of which is incorporated herein by reference. in some embodiments, a Cas9 variate have no specific PAM requirements. In some embodiments, a Cas9 variant, e.g. a SpCas9 variant has specificity for a NRNH PAM, wherein R is A or G and H is A, C, or T. In some embodiments, the SpCas9 variant has specificity for a PAM sequence AAA, TAA, CAA, GAA, TAT, GAT, or CAC. In some embodiments, the SpCas9 variant comprises an amino acid substitution at position 1114, 1134, 1135, 1137, 1139, 1151, 1180, 1188, 1211, 1218, 1219, 1221, 1249, 1256, 1264, 1290, 1318, 1317, 1320, 1321, 1323, 1332, 1333, 1335, 1337, or 1339 or a corresponding position thereof. In some embodiments, the SpCas9 variant comprises an amino acid substitution at position 1114, 1135, 1218, 1219, 1221, 1249, 1320, 1321, 1323, 1332, 1333, 1335, or 1337 or a corresponding position thereof. In some embodiments, the SpCas9 variant comprises an amino acid substitution at position 1114, 1134, 1135, 1137, 1139, 1151, 1180, 1188, 1211, 1219, 1221, 1256, 1264, 1290, 1318, 1317, 1320, 1323, 1333 or a corresponding position thereof. In some embodiments, the SpCas9 variant comprises an amino acid substitution at position 1114, 1131, 1135, 1150, 1156, 1180, 1191, 1218, 1219, 1221, 1227, 1249, 1253, 1286, 1293, 1320, 1321, 1332, 1335, 1339 or a corresponding position thereof. In some embodiments, the SpCas9 variant comprises an amino acid substitution at position 1114, 1127, 1135, 1180, 1207, 1219, 1234, 1286, 1301, 1332, 1335, 1337, 1338, 1349 or a corresponding position thereof. Exemplary amino acid substitutions and PAM specificity of SpCas9 variants are shown in Tables 2A-2D.

TABLE 2A
SpCas9 Variants

| | SpCas9 amino acid position | | | | | | | | | | | | |
|---|---|---|---|---|---|---|---|---|---|---|---|---|---|
| | 1114 | 1135 | 1218 | 1219 | 1221 | 1249 | 1320 | 1321 | 1323 | 1332 | 1333 | 1335 | 1337 |
| SpCas9 | R | D | G | E | Q | P | A | P | A | D | R | R | T |
| AAA | | N | | V | H | | | | | | G | | |
| AAA | | N | | V | H | | | | | | G | | |
| AAA | | | | V | | | | | | | G | | |
| TAA | G | N | | V | | | | | | | I | | |
| TAA | | N | | V | | | | | | | I | | A |
| TAA | G | N | | V | | | | | | | I | | A |
| CAA | | | | V | | | | | | | K | | |
| CAA | | N | | V | | | | | | | K | | |
| CAA | | N | | V | | | | | | | K | | |
| GAA | | | | V | H | | V | | | | K | | |
| GAA | | N | | V | | | V | | | | K | | |
| GAA | | | | V | H | | V | | | | K | | |
| TAT | | | S | V | H | S | | S | | | | L | |
| TAT | | | S | V | H | S | | S | | | | L | |
| TAT | | | S | V | H | S | | S | | | | L | |
| GAT | | | | V | | | | | | | I | | |
| GAT | | | | V | | | | | D | | | Q | |
| GAT | | | | V | | | | | D | | | Q | |
| CAC | | | | V | | | | | | N | | Q | N |
| CAC | | N | | V | | | | | | | | Q | N |
| CAC | | | | V | | | | | | N | | Q | N |

TABLE 2B

| | SpCas9 amino acid position | | | | | | | | | | | | | | | | | | |
|---|---|---|---|---|---|---|---|---|---|---|---|---|---|---|---|---|---|---|---|
| | 1114 | 1134 | 1135 | 1137 | 1139 | 1151 | 1180 | 1188 | 1211 | 1219 | 1221 | 1256 | 1264 | 1290 | 1318 | 1317 | 1320 | 1323 | 1333 |
| SpCas9 | R | F | D | P | V | K | D | K | K | E | Q | Q | H | V | L | N | A | A | R |
| GAA | | | | | | | | | | V | H | | | | | | V | | K |
| GAA | | | N | S | | | | | | V | | | | | | | V | D | K |
| GAA | | | N | | | | | | | V | H | | Y | | | | V | | K |
| CAA | | | N | | | | | | | V | H | | Y | | | | V | | K |
| CAA | G | | N | S | | | | | | V | H | | Y | | | | V | | K |
| CAA | | | N | | | | | R | | V | H | | | | | | V | | K |
| CAA | | | N | | | | G | | R | V | H | | Y | | | | V | | K |
| CAA | | | N | | | | | | | V | H | | Y | | | | V | | K |
| AAA | | | N | | | | G | | | V | H | R | Y | | | | V | D | K |
| CAA | G | | N | | | | G | | | V | H | | Y | | | | V | D | K |
| CAA | | L | N | | | | G | | | V | H | | Y | | | T | V | D | K |
| TAA | G | | N | | | | G | | | V | H | | Y | G | S | | V | D | K |
| TAA | G | | N | | | E | G | | | V | H | | Y | | S | | V | | K |
| TAA | G | | N | | | | G | | | V | H | | Y | | S | | V | D | K |
| TAA | G | | N | | | | G | | R | V | H | | | | | | V | | K |
| TAA | | | N | | | | G | | R | V | H | | Y | | | | V | | K |
| TAA | G | | N | | A | | G | | | V | H | | | | | | V | | K |
| TAA | G | | N | | | | | | | V | H | | | | | | V | | K |

TABLE 2C

| | SpCas9 amino acid position | | | | | | | | | | |
|---|---|---|---|---|---|---|---|---|---|---|---|
| | 1114 | 1131 | 1135 | 1150 | 1156 | 1180 | 1191 | 1218 | 1219 | 1221 | 1227 |
| SpCas9 | R | Y | D | E | K | D | K | G | E | Q | A |
| SacB.TAT | | | N | | | | N | | V | H | |
| SacB.TAT | | | N | | | | | S | V | H | |
| AAT | | | N | | | S | | | V | H | V |
| TAT | G | | N | | | G | | S | V | H | |
| TAT | G | | N | | | G | | S | V | H | |
| TAT | G | C | N | | | G | | S | V | H | |
| TAT | G | C | N | | | G | | S | V | H | |
| TAT | G | C | N | | | G | | S | V | H | |
| TAT | G | C | N | | E | G | | S | V | H | |
| TAT | G | C | N | V | | G | | S | V | H | |
| TAT | | C | N | | | G | | S | V | H | |
| TAT | G | C | N | | | G | | S | V | H | |

| | SpCas9 amino acid position | | | | | | | | |
|---|---|---|---|---|---|---|---|---|---|
| | 1249 | 1253 | 1286 | 1293 | 1320 | 1321 | 1332 | 1335 | 1339 |
| SpCas9 | P | E | N | A | A | P | D | R | T |
| SacB.TAT | | | | | V | S | | L | |
| SacB.TAT | S | | | | | S | G | L | |
| AAT | S | | K | T | | S | G | L | I |
| TAT | S | K | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |
| TAT | S | | | | | S | G | L | |

TABLE 2D

| | SpCas9 amino acid position | | | | | | | | | | | | | |
|---|---|---|---|---|---|---|---|---|---|---|---|---|---|---|
| | 1114 | 1127 | 1135 | 1180 | 1207 | 1219 | 1234 | 1286 | 1301 | 1332 | 1335 | 1337 | 1338 | 1349 |
| SpCas9 | R | D | D | D | E | E | N | N | P | D | R | T | S | H |
| SacB.CAC | | | N | | | V | | | | N | Q | N | | |
| AAC | G | | N | | | V | | | | N | Q | N | | |
| AAC | G | | N | | | V | | | | N | Q | N | | |
| TAC | G | | N | | | V | | | | N | Q | N | | |
| TAC | G | | N | | | V | | H | | N | Q | N | | |
| TAC | G | | N | | G | V | D | H | | N | Q | N | | |
| TAC | G | | N | | | V | | | | N | Q | N | | |
| TAC | G | G | N | E | | V | | H | | N | Q | N | | |
| TAC | G | | N | | | V | | H | | N | Q | N | | |
| TAC | G | | N | | | V | | | | N | Q | N | T | R |

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

In some embodiments, the napDNAbp is a circular permutant (e.g., SEQ ID NO: 257).

The crystal structure of Alicyclobaccillus acidoterrastris Cas12b/C2c1 (AacC2c1) has been reported in complex with a chimeric single-molecule guide RNA (sgRNA). See e.g., Liu et “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

In some embodiments, a napDNAbp refers to Cas12c. In some embodiments, the Cas12c protein is a Cas12c1 (SEQ ID NO: 266) or a variant of Cas12c1. In some embodiments, the Cas12 protein is a Cas12c2 (SEQ ID NO: 267) or a variant of Cas12c2. In some embodiments, the Cas12 protein is a Cas12c protein from Oleiphilus sp. H10009 OspCas12c; SEQ ID NO: 268) or a variant of OspCas12c. These Cas12c molecules have been described in Yan et al., “Functionally Diverse Type V CRISPR-Cas Systems,” Science, 2019 Jan. 4; 363: 88-91; the entire contents of which is hereby incorporated by reference. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12c1, Cas12c2, or OspCas12c protein. In some embodiments, the napDNAbp is a naturally-occurring Cas12c1, Cas12c2, or OspCas12c protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12c1, Cas12c2, or OspCas12c protein described herein. It should be appreciated that Cas12c1, Cas12c2, or OspCas12c from other bacterial species may also be used in accordance with the present disclosure.

In some embodiments, a napDNAbp refers to Cas12g, Cas12h, or Cas12i, which have been described in, for example, Yan et al., “Functionally Diverse Type V CRISPR-Cas Systems,” Science, 2019 Jan. 4; 363: 88-91; the entire contents of each is hereby incorporated by reference. Exemplary Cas12g, Cas12h, and Cas12i polypeptide sequences are provided in the Sequence Listing as SEQ ID NOs: 269-272. By aggregating more than 10 terabytes of sequence data, new classifications of Type V Cas proteins were identified that showed weak similarity to previously characterized Class V protein, including Cas12g, Cas12h, and Cas12i. In some embodiments, the Cas12 protein is a Cas12g or a variant of Cas12g. In some embodiments, the Cas12 protein is a Cas12h or a variant of Cas12h. In some embodiments, the Cas12 protein is a Cas12i or a variant of Cas12i. It should be appreciated that other RNA-guided DNA binding proteins may be used as a napDNAbp, and are within the scope of this disclosure. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12g, Cas12h, or Cas12i protein. In some embodiments, the napDNAbp is a naturally-occurring Cas12g, Cas12h, or Cas12i protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any Cas12g, Cas12h, or Cas12i protein described herein. It should be appreciated that Cas12g, Cas12h, or Cas12i from other bacterial species may also be used in accordance with the present disclosure. In some embodiments, the Cas12i is a Cas12i1 or a Cas12i2.

In some embodiments, the nucleic acid programmable DNA binding protein (napDNAbp) of any of the fusion proteins provided herein may be a Cas12j/Casϕ protein. Cas12j/Casϕ is described in Pausch et al., “CRISPR-Casϕ from huge phages is a hypercompact genome editor,” Science, 17 Jul. 2020, Vol. 369, Issue 6501, pp. 333-337, which is incorporated herein by reference in its entirety. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12j/Casϕ protein. In some embodiments, the napDNAbp is a naturally-occurring Cas12j/Casϕ protein. In some embodiments, the napDNAbp is a nuclease inactive (“dead”) Cas12j/Casϕ protein. It should be appreciated that Cas12j/Casϕ from other species may also be used in accordance with the present disclosure.

Fusion Proteins with Internal Insertion

Provided herein are fusion proteins comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbpin some embodiments, the heterologous polypeptide is a deaminase (e.g., cytidine of adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide. In some embodiments, the cytidine deaminase is an APOBEC deaminase (e.g., APOBEC1). In some embodiments, the adenosine deaminase is a TadA (e.g., TadA*7.10 or TadA*8). In some embodiments, the TadA is a TadA*8 or a TadA*9. TadA sequences (e.g., TadA7.10 or TadA*8) as described herein are suitable deaminases for the above-described fusion proteins.

In some embodiments, the fusion protein comprises the structure:

NH2-[N-terminal fragment of a napDNAbp]-[deaminase]-[C-terminal fragment of a napDNAbp]-COOH;
NH2-[N-terminal fragment of a Cas9]-[adenosine deaminase]-[C-terminal fragment of a Cas9]-COOH;
NH2-[N-terminal fragment of a Cas12]-[adenosine deaminase]-[C-terminal fragment of a Cas12]-COOH;
NH2-[N-terminal fragment of a Cas9]-[cytidine deaminase]-[C-terminal fragment of a Cas9]-COOH;
NH2-[N-terminal fragment of a Cas12]-[cytidine deaminase]-[C-terminal fragment of a Cas12]-COOH;
wherein each instance of “]-[” is an optional linker.

The deaminase can be a circular permutant deaminase. For example, the deaminase can be a circular permutant adenosine deaminase. In some embodiments, the deaminase is a circular permutant TadA, circularly permutated at amino acid residue 116, 136, or 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one or two deaminase. The two or more deaminases in a fusion protein can be an adenosine deaminase, a cytidine deaminase, or a combination thereof. The two or more deaminases can be homodimers or heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus 1 Cas9 (St1Cas9), or fragments or variants of any of the Cas9 polypeptides described herein.

In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:

NH2-[Cas9(adenosine deaminase)]-[cytidine deaminase]-COOH;
NH2-[cytidine deaminase]-[Cas9(adenosine deaminase)]-COOH;
NH2-[Cas9(cytidine deaminase)]-[adenosine deaminase]-COOH; or
NH2-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:

NH2-[Cas9(TadA*8)]-[cytidine deaminase]-COOH;
NH2-[cytidine deaminase]-[Cas9(TadA*8)]-COOH;
NH2-[Cas9(cytidine deaminase)]-[TadA*8]-COOH; or
NH2-[TadA*8]-[Cas9(cytidine deaminase)]-COOH.

In some embodiments, the “-” used in the general architecture above indicates the presence of an optional linker.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2c1 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, a cytidine deaminase (e.g., APOBEC1) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the cytidine deaminase is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 3 below:

TABLE 3
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of: RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an deaminase can be at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein described herein can effect targeted deamination with reduced deamination at non-target sites (e.g., off-target sites), such as reduced genome wide spurious deamination. The fusion protein described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein deaminates no more than two nucleobases within the range of an R-loop. In some embodiments, the deaminase of the fusion protein deaminates no more than three nucleobases within the range of the R-loop. In some embodiments, the deaminase of the fusion protein deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA: RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein can comprise more than one heterologous polypeptide. For example, the fusion protein can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n (SEQ ID NO: 1308), (GGGGS)n (SEQ ID NO: 109), (G)n, (EAAAK)n (SEQ ID NO: 1309), (GGS)n, SGSETPGTSESATPES (SEQ ID NO: 56). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the napDNAbp in the fusion protein is a Cas12 polypeptide, e.g., Cas12b/C2c1, or a fragment thereof. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 273) or GSSGSETPGTSESATPESSG (SEQ ID NO: 1310). In other embodiments, the linker is a rigid linker. In other embodiments of the above aspects, the linker is encoded by

(SEQ ID NO: 1311)
GGAGGCTCTGGAGGAAGC
or
(SEQ ID NO: 1312)
GGCTCTTCTGGATCTGAAACACCTGGCACAAGCGAGAGCGCCACCCCTGA
GAGCTCTGGC.

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:

NH2-[Cas12(adenosine deaminase)]-[cytidine deaminase]-COOH;

NH2-[cytidine deaminase]-[Cas12(adenosine deaminase)]-COOH;

NH2-[Cas12(cytidine deaminase)]-[adenosine deaminase]-COOH; or

NH2-[adenosine deaminase]-[Cas12(cytidine deaminase)]-COOH;

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA*7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:

N-[Cas12(TadA*8)]-[cytidine deaminase]-C;

N-[cytidine deaminase]-[Cas12(TadA*8)]-C;

N-[Cas12(cytidine deaminase)]-[TadA*8]-C; or

N-[TadA*8]-[Cas12(cytidine deaminase)]-C.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, Cas12i, or Cas12j/Casϕ. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b (SEQ ID NO: 259). In other embodiments, the Cas12 polypeptide has at least about 90% amino acid sequence identity to Bacillus hisashii Cas12b (SEQ ID NO: 260), Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b (SEQ ID NO: 265), Bacillus sp. V3-13 Cas12b (SEQ ID NO: 264), or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b. In embodiments, the Cas12 polypeptide contains BvCas12b (V4), which in some embodiments is expressed as 5′ mRNA Cap-5′ UTR-bhCas12b-STOP sequence-3′ UTR 120polyA tail (SEQ ID NOs: 261-263).

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, Cas12i, or Cas12j/Casϕ. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, Cas12i, or Cas12j/Casϕ. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, Cas12i, or Cas12j/Casϕ. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA (SEQ ID NO: 1313). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 1314). In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4 below.

TABLE 4
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., TadA*8.13) may be inserted into a BhCas12b to produce a fusion protein (e.g., TadA*8.13-BhCas12b) that effectively edits a nucleic acid sequence.

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 1315-1360.

In some embodiments, adenosine deaminase base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA). In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2) or tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1363-1370.

The adenosine deaminase can be derived from any suitable organism (e.g., E. coli). In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in TadA reference sequence, or a corresponding mutation in another adenosine deaminase. It should be appreciated, however, that additional deaminases may similarly be aligned to identify homologous amino acid residues that can be mutated as provided herein.

In some embodiments, the adenosine deaminase comprises an A106X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., ecTadA).

In some embodiments, the adenosine deaminase comprises a E155X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

It should also be appreciated that any of the mutations provided herein may be made individually or in any combination in ecTadA or another adenosine deaminase. For example, an adenosine deaminase may contain a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase: D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein may be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, 195X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, 195L, V102A, F104L, A106V, R107C, or R107H, or R107P, D108G, or D108N, or D108V, or D108A, or D108Y, K110I, M118K, N127S, A138V, F149Y, M151V, R153C, Q154L, 1156D, and/or K157R mutation in TadA reference sequence, or one or more corresponding mutations in another adenosine deaminase.

In some embodiments, the adenosine deaminase comprises one or more of a H8X, D108X, and/or N127X mutation in TadA reference sequence, or one or more corresponding mutations in another adenosine deaminase, where X indicates the presence of any amino acid. In some embodiments, the adenosine deaminase comprises one or more of a H8Y, D108N, and/or N127S mutation in TadA reference sequence, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M611, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, and D108X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of H8X, R126X, L68X, D108X, N127X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, D108X, A109X, N127X, and E155X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M611, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

In some embodiments, the adenosine deaminase comprises one or more of the or one or more corresponding mutations in another adenosine deaminase. In some embodiments, the adenosine deaminase comprises a D108N, D108G, or D108V mutation in TadA reference sequence, or corresponding mutations in another adenosine deaminase. In some embodiments, the adenosine deaminase comprises a A106V and D108N mutation in TadA reference sequence, or corresponding mutations in another adenosine deaminase. In some embodiments, the adenosine deaminase comprises R107C and D108N mutations in TadA reference sequence, or corresponding mutations in another adenosine deaminase. In some embodiments, the adenosine deaminase comprises a H8Y, D108N, N127S, D147Y, and Q154H mutation in TadA reference sequence, or corresponding mutations in another adenosine deaminase. In some embodiments, the adenosine deaminase comprises a H8Y, D108N, N127S, D147Y, and E155V mutation in TadA reference sequence, or corresponding mutations in another adenosine deaminase. In some embodiments, the adenosine deaminase comprises a D108N, D147Y, and E155V mutation in TadA reference sequence, or corresponding mutations in another adenosine deaminase. In some embodiments, the adenosine deaminase comprises a H8Y, D108N, and N127S mutation in TadA reference sequence, or corresponding mutations in another adenosine deaminase. In some embodiments, the adenosine deaminase comprises a A106V, D108N, D147Y, and E155V mutation in TadA reference sequence, or corresponding mutations in another adenosine deaminase (e.g., ecTadA).

In some embodiments, the adenosine deaminase comprises one or more of S2X, H8X, I49X, L84X, H123X, N127X, I156X, and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F, and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase.

In some embodiments, the adenosine deaminase comprises an I156X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five mutations selected from the group consisting of H8X, A106X, D108X, N127X, and K160X in TadA reference sequence, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase. In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five mutations selected from the group consisting of H8Y, A106T, D108N, N127S, and K160S in TadA reference sequence, or a corresponding mutation or mutations in another adenosine deaminase.

In some embodiments, the adenosine deaminase comprises one or more of a E25X, R26X, R107X, A142X, and/or A143X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q, and/or A143R mutation in TadA reference sequence, or one or more corresponding mutations in another adenosine deaminase. In some embodiments, the adenosine deaminase comprises one or more of the mutations described herein corresponding to TadA reference sequence, or one or more corresponding mutations in another adenosine deaminase.

In some embodiments, the adenosine deaminase comprises an E25X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase, where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q, and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, 149V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase.

In some embodiments, the adenosine deaminase comprises an N37X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an N37T or N37S mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an P48T or P48L mutation in TadA reference sequence, or a corresponding mutation in another adenosine deaminase.

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H or R51L mutation in TadA reference sequence, or a corresponding mutation in another adenosine deaminase.

In some embodiments, the adenosine deaminase comprises an S146X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an S146R or S146C mutation in TadA reference sequence, or a corresponding mutation in another adenosine deaminase.

In some embodiments, the adenosine deaminase comprises an K157X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase.

In some embodiments, the adenosine deaminase comprises an P48X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase.

In some embodiments, the adenosine deaminase comprises an A142X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase.

In some embodiments, the adenosine deaminase comprises an W23X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a W23R or W23L mutation in TadA reference sequence, or a corresponding mutation in another adenosine deaminase.

In some embodiments, the adenosine deaminase comprises an R152X mutation in TadA reference sequence, or a corresponding mutation in another adenosine deaminase, where X indicates any amino acid other than the corresponding amino acid in the wild-type adenosine deaminase. In some embodiments, the adenosine deaminase comprises a R152P or R52H mutation in TadA reference sequence, or a corresponding mutation in another adenosine deaminase.

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:

### (A106V_D108N),

### (R107C_D108N),

### (H8Y_D108N_N127S_D147Y_Q154H),

### (H8Y_D108N_N127S_D147Y_E155V),

### (D108N_D147Y_E155V),

### (H8Y_D108N_N127S),

### (H8Y_D108N_N127S_D147Y_Q154H),

### (A106V_D108N_D147Y_E155V),

### (D108Q_D147Y_E155V),

### (D108M_D147Y_E155V),

### (D108L_D147Y_E155V),

### (D108K_D147Y_E155V),

### (D108I_D147Y_E155V),

### (D108F_D147Y_E155V),

### (A106V_D108N_D147Y),

### (A106V_D108M_D147Y_E155V),

### (E59A_A106V_D108N_D147Y_E155V),

(E59A cat dead_A106V_D108N_D147Y_E155V),

### (L84F_A106V_D108N_H123Y_D147Y_E155V_I156Y),

### (L84F_A106V_D108N_H123Y_D147Y_E155V_I156F),

### (D103A_D104N),

### (G22P_D103A_D104N),

### (D103A_D104N_S138A),

### (R26G_L84F_A106V_R107H_D108N_H123Y_A142N_A143D_D147Y_E155V_I156F),

### (E25G_R26G_L84F_A106V_R107H_D108N_H123Y_A142N_A143D_D147Y_E155V_I156F),

### (E25D_R26G_L84F_A106V_R107K_D108N_H123Y_A142N_A143G_D147Y_E155V_I156F),

### (R26Q_L84F_A106V_D108N_H123Y_A142N_D147Y_E155V_I156F),

### (E25M_R26G_L84F_A106V_R107P_D108N_H123Y_A142N_A143D_D147Y_E155V_I156F),

### (R26C_L84F_A106V_R107H_D108N_H123Y_A142N_D147Y_E155V_I156F),

### (L84F_A106V_D108N_H123Y_A142N_A143L_D147Y_E155V_I156F),

### (R26G_L84F_A106V_D108N_H123Y_A142N_D147Y_E155V_I156F),

### (E25A_R26G_L84F_A106V_R107N_D108N_H123Y_A142N_A143E_D147Y_E155V_I156F),

### (R26G_L84F_A106V_R107H_D108N_H123Y_A142N_A143D_D147Y_E155V_I156F),

### (A106V_D108N_A142N_D147Y_E155V),

### (R26G_A106V_D108N_A142N_D147Y_E155V),

### (E25D_R26G_A106V_R107K_D108N_A142N_A143G_D147Y_E155V),

### (R26G_A106V_D108N_R107H_A142N_A143D_D147Y_E155V),

### (E25D_R26G_A106V_D108N_A142N_D147Y_E155V),

### (A106V_R107K_D108N_A142N_D147Y_E155V),

### (A106V_D108N_A142N_A143G_D147Y_E155V),

### (A106V_D108N_A142N_A143L_D147Y_E155V),

### (H36L_R51L_L84F_A106V_D108N_H123Y_S146C_D147Y_E155V_I156F_K157N),

### (N37T_P48T_M70L_L84F_A106V_D108N_H123Y_D147Y_I49V_E155V_I156F),

### (N37S_L84F_A106V_D108N_H123Y_D147Y_E155V_I156F_K161T),

### (H36L_L84F_A106V_D108N_H123Y_D147Y_Q154H_E155V_I156F),

### (N72S_L84F_A106V_D108N_H123Y_S146R_D147Y_E155V_I156F),

### (H36L_P48L_L84F_A106V_D108N_H123Y_E134G_D147Y_E155V_I156F),

### (H36L_L84F_A106V_D108N_H123Y_D147Y_E155V_I156F_K157N)

### (H36L_L84F_A106V_D108N_H123Y_S146C_D147Y_E155V_I156F),

### (L84F_A106V_D108N_H123Y_S146R_D147Y_E155V_I156F_K161T),

### (N37S_R51H_D77G_L84F_A106V_D108N_H123Y_D147Y_E155V_I156F),

### (R51L_L84F_A106V_D108N_H123Y_D147Y_E155V_I156F_K157N),

### (D24G_Q71R_L84F_H96L_A106V_D108N_H123Y_D147Y_E155V_I156F_K160E),

### (H36L_G67V_L84F_A106V_D108N_H123Y_S146T_D147Y_E155V_I156F),

### (Q71L_L84F_A106V_D108N_H123Y_L137M_A143E_D147Y_E155V_I156F),

### (E25G_L84F_A106V_D108N_H123Y_D147Y_E155V_I156F_Q159L),

### (L84F_A91T_F1041_A106V_D108N_H123Y_D147Y_E155V_I156F),

### (N72D_L84F_A106V_D108N_H123Y_G125A_D147Y_E155V_I156F),

### (P48S_L84F_S97C_A106V_D108N_H123Y_D147Y_E155V_I156F),

### (W23G_L84F_A106V_D108N_H123Y_D147Y_E155V_I156F),

### (D24G_P48L_Q71R_L84F_A106V_D108N_H123Y_D147Y_E155V_I156F_Q159L),

### (L84F_A106V_D108N_H123Y_A142N_D147Y_E155V_I156F),

### (H36L_R51L_L84F_A106V_D108N_H123Y_A142N_S146C_D147Y_E155V_I156F_K157N),

### (N37S_L84F_A106V_D108N_H123Y_A142N_D147Y_E155V_I156F_K161T),

### (L84F_A106V_D108N_D147Y_E155V_I156F),

### (R51L_L84F_A106V_D108N_H123Y_S146C_D147Y_E155V_I156F_K157N_K161T),

### (L84F_A106V_D108N_H123Y_S146C_D147Y_E155V_I156F_K161T),

### (L84F_A106V_D108N_H123Y_S146C_D147Y_E155V_I156F_K157N_K160E_K161T),

### (L84F_A106V_D108N_H123Y_S146C_D147Y_E155V_I156F_K157N_K160E),

### (R74Q_L84F_A106V_D108N_H123Y_D147Y_E155V_I156F),

### (R74A_L84F_A106V_D108N_H123Y_D147Y_E155V_I156F),

### (L84F_A106V_D108N_H123Y_D147Y_E155V_I156F),

### (R74Q_L84F_A106V_D108N_H123Y_D147Y_E155V_I156F),

### (L84F_R98Q_A106V_D108N_H123Y_D147Y_E155V_I156F),

### (L84F_A106V_D108N_H123Y_R129Q_D147Y_E155V_I156F),

### (P48S_L84F_A106V_D108N_H123Y_A142N_D147Y_E155V_I156F),

### (P48S_A142N),

### (P48T_I49V_L84F_A106V_D108N_H123Y_A142N_D147Y_E155V_I156F_L157N),

### (P48T_I49V_A142N),

### (H36L_P48S_R51L_L84F_A106V_D108N_H123Y_S146C_D147Y_E155V_I156F_K157N),

### (H36L_P48S_R51L_L84F_A106V_D108N_H123Y_S146C_A142N_D147Y_E155V_I156F

### (H36L_P48T_I49V_R51L_L84F_A106V_D108N_H123Y_S146C_D147Y_E155V_I156F_K157N),

### (H36L_P48T_I49V_R51L_L84F_A106V_D108N_H123Y_A142N_S146C_D147Y_E155V_I156F_K157N),

### (H36L_P48A_R51L_L84F_A106V_D108N_H123Y_S146C_D147Y_E155V_I156F_K157N),

### (H36L_P48A_R51L_L84F_A106V_D108N_H123Y_A142N_S146C_D147Y_E155V_I156F_K157N),

### (H36L_P48A_R51L_L84F_A106V_D108N_H123Y_S146C_A142N_D147Y_E155V_I156F_K157N),

### (W23L_H36L_P48A_R51L_L84F_A106V_D108N_H123Y_S146C_D147Y_E155V_I156F_K157N),

### (W23R_H36L_P48A_R51L_L84F_A106V_D108N_H123Y_S146C_D147Y_E155V_I156F_K157N),

### (W23L_H36L_P48A_R51L_L84F_A106V_D108N_H123Y_S146R_D147Y_E155V_I156F_K161T),

### (H36L_P48A_R51L_L84F_A106V_D108N_H123Y_S146C_D147Y_R152H_E155V_I156F_K157N),

### (H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_A142A_S146C_D147Y_E155V_I156F_K157N),

### (W23L_H36L_P48A_R51L_L84F_A106V_D108N_H123Y_A142A_S146C_D147Y_R152P_E155V_I156F_K157N),

### (W23L_H36L_P48A_R51L_L84F_A106V_D108N_H123Y_S146R_D147Y_E155V_I156F_K161T),

### (W23R_H36L_P48A_R51L_L84F_A106V_D108N_H123Y_S146C_D147Y_R152P_E155V_I156F_K157N),

### (H36L_P48A_R51L_L84F_A106V_D108N_H123Y_A142N_S146C_D147Y_R152P_E155V_I156F_K157N).

In some embodiments, the TadA deaminase is TadA variant. In some embodiments, the TadA variant is TadA*7.10. In particular embodiments, the fusion proteins comprise a single TadA*7.10 domain (e.g., provided as a monomer). In other embodiments, the fusion protein comprises TadA*7.10 and TadA(wt), which are capable of forming heterodimers. In one embodiment, a fusion protein of the invention comprises a wild-type TadA linked to TadA*7.10, which is linked to Cas9 nickase.

In some embodiments, TadA*7.10 comprises at least one alteration. In some embodiments, the adenosine deaminase comprises an alteration in the following sequence:

TadA*7.10
(SEQ ID NO: 8)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, TadA*7.10 comprises an alteration at amino acid 82 and/or 166. In particular embodiments, TadA*7.10 comprises one or more of the following alterations: Y147T, Y147R, Q154S, Y123H, V82S, T166R, and/or Q154R. In other embodiments, a variant of TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R.

In some embodiments, an adenosine deaminase variant (e.g., TadA*8) comprises a deletion. In some embodiments, an adenosine deaminase variant comprises a deletion of the C terminus. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, an adenosine deaminase variant (e.g., TadA*8) is a monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a homodimer comprising two adenosine deaminase domains (e.g., TadA*8) each having one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a heterodimer of a wild-type adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a wild-type adenosine deaminase domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a heterodimer of a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer of a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, an adenosine deaminase is a TadA*8. In one embodiment, an adenosine deaminase is a TadA*8 that comprises or consists essentially of the following sequence or a fragment thereof having adenosine deaminase activity:

(SEQ ID NO: 12)
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RWFGVRNAKTGAAGSLMDVLHYPGMNHRVEITEGILADECAALLCTFFRM
PRQVFNAQKKAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In other embodiments, a base editor of the disclosure comprising an adenosine deaminase variant (e.g., TadA*8) monomer comprising one or more of the following alterations: R26C, V88A, A109S, T111R, D119N, H122N, Y147D, F149Y, T166I and/or D167N, relative to TadA*7.10, the TadA reference sequence, or a corresponding mutation in another TadA. In other embodiments, the adenosine deaminase variant (TadA*8) monomer comprises a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, a base editor comprises a heterodimer of a wild-type adenosine deaminase domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a wild-type adenosine deaminase domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In other embodiments, a base editor comprises a heterodimer of a TadA*7.10 domain and an adenosine deaminase variant domain (e.g., TadA*8) comprising one or more of the following alterations R26C, V88A, A109S, T111R, D119N, H122N, Y147D, F149Y, T166I and/or D167N, relative to TadA*7.10, the TadA reference sequence, or a corresponding mutation in another TadA. In other embodiments, the base editor comprises a heterodimer of a TadA*7.10 domain and an adenosine deaminase variant domain (e.g., TadA*8) comprising a combination of alterations selected from the group of: R26C+A109S+T111R+D119N+H122N+Y147D+F149Y+T166I+D167N; V88A+A109S+T111R+D119N+H122N+F149Y+T166I+D167N; R26C+A109S+T111R+D119N+H122N+F149Y+T166I+D167N; V88A+T111R+D119N+F149Y; and A109S+T111R+D119N+H122N+Y147D+F149Y+T166I+D167N, relative to TadA*7.10, the TadA reference sequence, or a corresponding mutation in another TadA.

In some embodiments, the TadA*8 is a variant as shown in Table 5. Table 5 shows certain amino acid position numbers in the TadA amino acid sequence and the amino acids present in those positions in the TadA-7.10 adenosine deaminase. Table 5 also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e.

TABLE 5
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| | TadA-8a | C | | S | R | N | N | D | Y | I | N |
| | TadA-8b | | A | S | R | N | N | | Y | I | N |
| PACE | TadA-8c | C | | S | R | N | N | | Y | I | N |
| | TadA-8d | | A | | R | N | | | Y | | |
| | TadA-8e | | | S | R | N | N | D | Y | I | N |

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the fusion protein comprises TadA*8 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| (SEQ ID NO: 8) | |
|---|---|
| | MSEVEFSHEY WMRHALTLAK RARDEREVPV GAVLVLNNRV |
| | IGEGWNRAIG ⁵⁰ LHDPTAHAEI MALRQGGLVM QNYRLIDATL |
| | YVTFEPCVMC AGAMIHSRIG ¹⁰⁰ RVVFGVRNAK TGAAGSLMDV |
| | LHYPGMNHRV EITEGILADE CAALLCYFFR ¹⁵⁰ MPRQVFAAQK |
| | KAQSSTD |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein of the invention comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

In particular embodiments, the fusion proteins comprise a single (e.g., provided as a monomer) TadA*8. In some embodiments, the TadA*8 is linked to a Cas9 nickase. In some embodiments, the fusion proteins of the invention comprise as a heterodimer of a wild-type TadA (TadA(wt)) linked to a TadA*8. In other embodiments, the fusion proteins of the invention comprise as a heterodimer of a TadA*7.10 linked to a TadA*8. In some embodiments, the base editor is ABE8 comprising a TadA*8 variant monomer. In some embodiments, the base editor is ABE8 comprising a heterodimer of a TadA*8 and a TadA(wt). In some embodiments, the base editor is ABE8 comprising a heterodimer of a TadA*8 and TadA*7.10. In some embodiments, the base editor is ABE8 comprising a heterodimer of a TadA*8. In some embodiments, the TadA*8 is selected from Table 11, 13 or 14. In some embodiments, the ABE8 is selected from Table 13, 14 or 16.

In some embodiments, the adenosine deaminase is a TadA*9 variant. In some embodiments, the adenosine deaminase is a TadA*9 variant selected from the variants described below and with reference to the following sequence (termed TadA*7.10):

(SEQ ID NO: 8)
MSEVEFSHEY WMRHALTLAK RARDEREVPV GAVLVLNNRV
IGEGWNRAIG LHDPTAHAEI MALRQGGLVM QNYRLIDATL
YVTFEPCVMC AGAMIHSRIG RVVFGVRNAK TGAAGSLMDV
LHYPGMNHRV EITEGILADE CAALLCYFFR MPRQVFNAQK
KAQSSTD.

In some embodiments, an adenosine deaminase comprises one or more of the following alterations: R21N, R23H, E25F, N38G, L51W, P54C, M70V, Q71M, N72K, Y73S, V82T, M94V, P124W, T133K, D139L, D139M, C146R, and A158K. The one or more alternations are shown in the sequence above in underlining and bold font.

In some embodiments, an adenosine deaminase comprises one or more of the following combinations of alterations: V82S+Q154R+Y147R; V82S+Q154R+Y123H; V82S+Q154R+Y147R+Y123H; Q154R+Y147R+Y123H+I76Y+V82S; V82S+I76Y; V82S+Y147R; V82S+Y147R+Y123H; V82S+Q154R+Y123H; Q154R+Y147R+Y123H+I76Y; V82S+Y147R; V82S+Y147R+Y123H; V82S+Q154R+Y123H; V82S+Q154R+Y147R; V82S+Q154R+Y147R; Q154R+Y147R+Y123H+I76Y; Q154R+Y147R+Y123H+I76Y+V82S; I76Y_V82S_Y123H_Y147R_Q154R; Y147R+Q154R+H123H; and V82S+Q154R.

In some embodiments, an adenosine deaminase comprises one or more of the following combinations of alterations: E25F+V82S+Y123H, T133K+Y147R+Q154R; E25F+V82S+Y123H+Y147R+Q154R; L51W+V82S+Y123H+C146R+Y147R+Q154R; Y73S+V82S+Y123H+Y147R+Q154R; P54C+V82S+Y123H+Y147R+Q154R; N38G+V82T+Y123H+Y147R+Q154R; N72K+V82S+Y123H+D139L+Y147R+Q154R; E25F+V82S+Y123H+D139M+Y147R+Q154R; Q71M+V82S+Y123H+Y147R+Q154R; E25F+V82S+Y123H+T133K+Y147R+Q154R; E25F+V82S+Y123H+Y147R+Q154R; V82S+Y123H+P124W+Y147R+Q154R; L51W+V82S+Y123H+C146R+Y147R+Q154R; P54C+V82S+Y123H+Y147R+Q154R; Y73S+V82S+Y123H+Y147R+Q154R; N38G+V82T+Y123H+Y147R+Q154R; R23H+V82S+Y123H+Y147R+Q154R; R21N+V82S+Y123H+Y147R+Q154R; V82S+Y123H+Y147R+Q154R+A158K; N72K+V82S+Y123H+D139L+Y147R+Q154R; E25F+V82S+Y123H+D139M+Y147R+Q154R; and M70V+V82S+M94V+Y123H+Y147R+Q154R

In some embodiments, an adenosine deaminase comprises one or more of the following combinations of alterations: Q71M+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; E25F+I76Y+V82S+Y123H+Y147R+Q154R; I76Y+V82T+Y123H+Y147R+Q154R; N38G+I76Y+V82S+Y123H+Y147R+Q154R; R23H+I76Y+V82S+Y123H+Y147R+Q154R; P54C+I76Y+V82S+Y123H+Y147R+Q154R; R21N+I76Y+V82S+Y123H+Y147R+Q154R; I76Y+V82S+Y123H+D139M+Y147R+Q154R; Y73S+I76Y+V82S+Y123H+Y147R+Q154R; and V82S+Q154R; N72K_V82S+Y123H+Y147R+Q154R; Q71M_V82S+Y123H+Y147R+Q154R; V82S+Y123H+T133K+Y147R+Q154R; V82S+Y123H+T133K+Y147R+Q154R+A158K; M70V+Q71M+N72K+V82S+Y123H+Y147R+Q154R; N72K_V82S+Y123H+Y147R+Q154R; Q71M_V82S+Y123H+Y147R+Q154R; M70V+V82S+M94V+Y123H+Y147R+Q154R; V82S+Y123H+T133K+Y147R+Q154R; V82S+Y123H+T133K+Y147R+Q154R+A158K; and M70V+Q71M+N72K+V82S+Y123H+Y147R+Q154R. In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, the TadA*9 variant comprises the alterations described in Table 17 as described herein. In some embodiments, the TadA*9 variant is a monomer. In some embodiments, the TadA*9 variant is a heterodimer with a wild-type TadA adenosine deaminase. In some embodiments, the TadA*9 variant is a heterodimer with another TadA variant (e.g., TadA*8, TadA*9). Additional details of TadA*9 adenosine deaminases are described in International PCT Application No. PCT/2020/049975, which is incorporated herein by reference for its entirety.

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### C to T Editing

In some embodiments, a base editor disclosed herein comprises a fusion protein comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion of an APOBEC1 deaminase. In some embodiments, a deaminase incorporated into a base editor comprises all or a portion of APOBEC2 deaminase. In some embodiments, a deaminase incorporated into a base editor comprises all or a portion of is an APOBEC3 deaminase. In some embodiments, a deaminase incorporated into a base editor comprises all or a portion of an APOBEC3A deaminase. In some embodiments, a deaminase incorporated into a base editor comprises all or a portion of APOBEC3B deaminase. In some embodiments, a deaminase incorporated into a base editor comprises all or a portion of APOBEC3C deaminase. In some embodiments, a deaminase incorporated into a base editor comprises all or a portion of APOBEC3D deaminase. In some embodiments, a deaminase incorporated into a base editor comprises all or a portion of APOBEC3E deaminase. In some embodiments, a deaminase incorporated into a base editor comprises all or a portion of APOBEC3F deaminase. In some embodiments, a deaminase incorporated into a base editor comprises all or a portion of APOBEC3G deaminase. In some embodiments, a deaminase incorporated into a base editor comprises all or a portion of APOBEC3H deaminase. In some embodiments, a deaminase incorporated into a base editor comprises all or a portion of APOBEC4 deaminase. In some embodiments, a deaminase incorporated into a base editor comprises all or a portion of activation-induced deaminase (AID). In some embodiments a deaminase incorporated into a base editor comprises all or a portion of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, cow, dog, rat, or mouse. In some embodiments, the deaminase domain of the base editor is derived from rat (e.g., rat APOBEC1). In some embodiments, the deaminase domain of the base editor is human APOBEC1. In some embodiments, the deaminase domain of the base editor is pmCDA1.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins described herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor can comprise one or more mutations selected from the group consisting of H121X, H122X, R126X, R126X, R118X, W90X, W90X, and R132X of rAPOBEC1, or one or more corresponding mutations in another APOBEC deaminase, wherein X is any amino acid. In some embodiments, an APOBEC deaminase incorporated into a base editor can comprise one or more mutations selected from the group consisting of H121R, H122R, R126A, R126E, R118A, W90A, W90Y, and R132E of rAPOBEC1, or one or more corresponding mutations in another APOBEC deaminase.

In some embodiments, an APOBEC deaminase incorporated into a base editor can comprise one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor can comprise a H121R and a H122R mutation of rAPOBEC1, or one or more corresponding mutations in another APOBEC deaminase. In some embodiments an APOBEC deaminase incorporated into a base editor can comprise an APOBEC deaminase comprising a R126A mutation of rAPOBEC1, or one or more corresponding mutations in another APOBEC deaminase. In some embodiments, an APOBEC deaminase incorporated into a base editor can comprise an APOBEC deaminase comprising a R126E mutation of rAPOBEC1, or one or more corresponding mutations in another APOBEC deaminase. In some embodiments, an APOBEC deaminase incorporated into a base editor can comprise an APOBEC deaminase comprising a R118A mutation of rAPOBEC1, or one or more corresponding mutations in another APOBEC deaminase. In some embodiments, an APOBEC deaminase incorporated into a base editor can comprise an APOBEC deaminase comprising a W90A mutation of rAPOBEC1, or one or more corresponding mutations in another APOBEC deaminase. In some embodiments, an APOBEC deaminase incorporated into a base editor can comprise an APOBEC deaminase comprising a W90Y mutation of rAPOBEC1, or one or more corresponding mutations in another APOBEC deaminase. In some embodiments, an APOBEC deaminase incorporated into a base editor can comprise an APOBEC deaminase comprising a R132E mutation of rAPOBEC1, or one or more corresponding mutations in another APOBEC deaminase. In some embodiments an APOBEC deaminase incorporated into a base editor can comprise an APOBEC deaminase comprising a W90Y and a R126E mutation of rAPOBEC1, or one or more corresponding mutations in another APOBEC deaminase. In some embodiments, an APOBEC deaminase incorporated into a base editor can comprise an APOBEC deaminase comprising a R126E and a R132E mutation of rAPOBEC1, or one or more corresponding mutations in another APOBEC deaminase. In some embodiments, an APOBEC deaminase incorporated into a base editor can comprise an APOBEC deaminase comprising a W90Y and a R132E mutation of rAPOBEC1, or one or more corresponding mutations in another APOBEC deaminase. In some embodiments, an APOBEC deaminase incorporated into a base editor can comprise an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor can comprise an APOBEC deaminase comprising a D316R and a D317R mutation of hAPOBEC3G, or one or more corresponding mutations in another APOBEC deaminase. In some embodiments, any of the fusion proteins provided herein comprise an APOBEC deaminase comprising a R320A mutation of hAPOBEC3G, or one or more corresponding mutations in another APOBEC deaminase. In some embodiments, an APOBEC deaminase incorporated into a base editor can comprise an APOBEC deaminase comprising a R320E mutation of hAPOBEC3G, or one or more corresponding mutations in another APOBEC deaminase. In some embodiments, an APOBEC deaminase incorporated into a base editor can comprise an APOBEC deaminase comprising a R313A mutation of hAPOBEC3G, or one or more corresponding mutations in another APOBEC deaminase. In some embodiments, an APOBEC deaminase incorporated into a base editor can comprise an APOBEC deaminase comprising a W285A mutation of hAPOBEC3G, or one or more corresponding mutations in another APOBEC deaminase. In some embodiments, an APOBEC deaminase incorporated into a base editor can comprise an APOBEC deaminase comprising a W285Y mutation of hAPOBEC3G, or one or more corresponding mutations in another APOBEC deaminase. In some embodiments, an APOBEC deaminase incorporated into a base editor can comprise an APOBEC deaminase comprising a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R320E mutation of hAPOBEC3G, or one or more corresponding mutations in another APOBEC deaminase. In some embodiments, an APOBEC deaminase incorporated into a base editor can comprise an APOBEC deaminase comprising a R320E and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion of an APOBEC1 deaminase.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Deaminases

In some embodiments, the fusion proteins of the invention comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

A fusion protein of the invention second protein comprises two or more nucleic acid editing domains.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. See e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference).

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

In an embodiment, a guide polynucleotide described herein can be RNA or DNA. In one embodiment, the guide polynucleotide is a gRNA. An RNA/Cas complex can assist in “guiding” a Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M. et al., Science 337:816-821(2012), the entire contents of which is hereby incorporated by reference.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

A guide polynucleotide may include natural or non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 224-230, 223, 3000, and 243-245. Thus, a skilled artisan can change the genomic target of the Cas protein specificity is partially determined by how specific the gRNA targeting sequence is for the genomic target compared to the rest of the genome.

In other embodiments, a guide polynucleotide can comprise both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion of multiple separate molecules that are for instance hybridized along a region of complementarity. In some embodiments, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

The guide polynucleotides can be synthesized chemically, synthesized enzymatically, or a combination thereof. For example, the gRNA can be synthesized using standard phosphoramidite-based solid-phase synthesis methods. Alternatively, the gRNA can be synthesized in vitro by operably linking DNA encoding the gRNA to a promoter control sequence that is recognized by a phage RNA polymerase. Examples of suitable phage promoter sequences include T7, T3, SP6 promoter sequences, or variations thereof. In embodiments in which the gRNA comprises two separate molecules (e.g., crRNA and tracrRNA), the crRNA can be chemically synthesized and the tracrRNA can be enzymatically synthesized.

A gRNA molecule can be transcribed in vitro.

A guide polynucleotide may be expressed, for example, by a DNA that encodes the gRNA, e.g., a DNA vector comprising a sequence encoding the gRNA. The gRNA may be encoded alone or together with an encoded base editor. Such DNA sequences may be introduced into an expression system, e.g., a cell, together or separately. For example, DNA sequences encoding a polynucleotide programmable nucleotide binding domain and a gRNA may be introduced into a cell, each DNA sequence can be part of a separate molecule (e.g., one vector containing the polynucleotide programmable nucleotide binding domain coding sequence and a second vector containing the gRNA coding sequence) or both can be part of a same molecule (e.g., one vector containing coding (and regulatory) sequence for both the polynucleotide programmable nucleotide binding domain and the gRNA). An RNA can be transcribed from a synthetic DNA molecule, e.g., a gBlocks® gene fragment.

A gRNA or a guide polynucleotide can comprise three regions: a first region at the 5′ end that can be complementary to a target site in a chromosomal sequence, a second internal region that can form a stem loop structure, and a third 3′ region that can be single-stranded. A first region of each gRNA can also be different such that each gRNA guides a fusion protein to a specific target site. Further, second and third regions of each gRNA can be identical in all gRNAs.

A first region of a gRNA or a guide polynucleotide can be complementary to sequence at a target site in a chromosomal sequence such that the first region of the gRNA can base pair with the target site. In some cases, a first region of a gRNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a gRNA and a target site in a chromosomal sequence can be or can be about 10, 11, 12, 13, 14, 15, 16, 17, 18, 19, 20, 22, 23, 24, 25, or more nucleotides in length. Sometimes, a first region of a gRNA can be or can be about 19, 20, or 21 nucleotides in length.

A gRNA or a guide polynucleotide can also comprise a second region that forms a secondary structure. For example, a secondary structure formed by a gRNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A gRNA or a guide polynucleotide can also comprise a third region at the 3′ end that can be essentially single-stranded. For example, a third region is sometimes not complementarity to any chromosomal sequence in a cell of interest and is sometimes not complementarity to the rest of a gRNA. Further, the length of a third region can vary. A third region can be more than or more than about 4 nucleotides in length. For example, the length of a third region can range from or from about 5 to 60 nucleotides in length.

A gRNA or a guide polynucleotide can target any exon or intron of a gene target. In some cases, a guide can target exon 1 or 2 of a gene, in other cases; a guide can target exon 3 or 4 of a gene. In some embodiments, a composition comprises multiple gRNAs that all target the same exon or multiple gRNAs that target different exons. An exon and/or an intron of a gene can be targeted.

A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

Methods for selecting, designing, and validating guide polynucleotides, e.g., gRNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on single strand DNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a gRNA for use with Cas9s may be identified using a DNA sequence searching algorithm. gRNA design is carried out using custom gRNA design software based on the public tool cas-offinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of gRNAs, e.g., crRNAs, are ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

A gRNA can then be introduced into a cell or embryo as an RNA molecule or a non-RNA nucleic acid molecule, e.g., DNA molecule. In one embodiment, a DNA encoding a gRNA is operably linked to promoter control sequence for expression of the gRNA in a cell or embryo of interest. A RNA coding sequence can be operably linked to a promoter sequence that is recognized by RNA polymerase III (Pol III). Plasmid vectors that can be used to express gRNA include, but are not limited to, px330 vectors and px333 vectors. In some cases, a plasmid vector (e.g., px333 vector) can comprise at least two gRNA-encoding DNA sequences. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a gRNA can also be linear. A DNA molecule encoding a gRNA or a guide polynucleotide can also be circular.

In some embodiments, a reporter system is used for detecting base-editing activity and testing candidate guide polynucleotides. In some embodiments, a reporter system comprises a reporter gene based assay where base editing activity leads to expression of the reporter gene. For example, a reporter system may include a reporter gene comprising a deactivated start codon, e.g., a mutation on the template strand from 3′-TAC-S′ to 3′-CAC-S′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide can comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

A guide polynucleotide can be transferred into a cell by transfecting the cell with an isolated gRNA or a plasmid DNA comprising a sequence coding for the guide RNA and a promoter. A gRNA or a guide polynucleotide can also be transferred into a cell in other way, such as using virus-mediated gene delivery. A gRNA or a guide polynucleotide can be isolated. For example, a gRNA can be transfected in the form of an isolated RNA into a cell or organism. A gRNA can be prepared by in vitro transcription using any in vitro transcription system known in the art. A gRNA can be transferred to a cell in the form of isolated RNA rather than in the form of plasmid comprising encoding sequence for a gRNA.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or “-end of a gRNA which can inhibit exonuclease degradation. In some cases, phosphorothioate bonds can be added throughout an entire gRNA to reduce attack by endonucleases.

In some embodiments, the guide RNA is designed to disrupt a splice site (i.e., a splice acceptor (SA) or a splice donor (SD). In some embodiments, the guide RNA is designed such that the base editing results in a premature STOP codon.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence is essential for target binding, but the exact sequence depends on a type of Cas protein. The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities.

For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 6 below.

TABLE 6
Cas9 proteins and corresponding
PAM sequences

| Variant | PAM |
|---|---|
| spCas9 | NGG |
| spCas9-VRQR | NGA |
| spCas9-VRER | NGCG |
| xCas9(sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′(TTTV) |
| SpyMac | 5′-NAA-3′ |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”).

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 7A and 7B below.

TABLE 7A
NGT PAM Variant Mutations at residues 1219,
1335, 1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 7B
NGT PAM Variant Mutations at residues
1135, 1136, 1218, 1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Table 7A and Table 7B. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 8 below.

TABLE 8
NGT PAM Variant Mutations at residues
1219, 1335, 1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 9 below.

TABLE 9
NGT PAM variants

| | NGTN | | | | | | | |
|---|---|---|---|---|---|---|---|---|
| | vari- | | | | | | | |
| | ant | D1135 | S1136 | G1218 | E1219 | A1322R | R1335 | T1337 |
| Vari- | LRKIQK | L | R | K | I | — | Q | K |
| ant 1 | | | | | | | | |
| Vari- | LRSVQK | L | R | S | V | — | Q | K |
| ant 2 | | | | | | | | |
| Vari- | LRSVQL | L | R | S | V | — | Q | L |
| ant 3 | | | | | | | | |
| Vari- | LRKIRQK | L | R | K | I | R | Q | K |
| ant 4 | | | | | | | | |
| Vari- | LRSVRQK | L | R | S | V | R | Q | K |
| ant 5 | | | | | | | | |
| Vari- | LRSVRQL | L | R | S | V | R | Q | L |
| ant 6 | | | | | | | | |

In some embodiments the NGTN variant is variant 1. In some embodiments, the NGTN variant is variant 2. In some embodiments, the NGTN variant is variant 3. In some embodiments, the NGTN variant is variant 4. In some embodiments, the NGTN variant is variant 5. In some embodiments, the NGTN variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPR1 and 5′-NGGNG for CRISPR3) and Neisseria meningitidis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

In some embodiments, engineered SpCas9 variants are capable of recognizing protospacer adjacent motif (PAM) sequences flanked by a 3′ H (non-G PAM) (see Tables 2A-2D). In some embodiments, the SpCas9 variants recognize NRNH PAMs (where R is A or G and H is A, C or T). In some embodiments, the non-G PAM is NRRH, NRTH, or NRCH (see e.g., Miller, S. M., et al. Continuous evolution of SpCas9 variants compatible with non-G PAMs, Nat. Biotechnol. (2020), the contents of which is incorporated herein by reference in its entirety).

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

The sequence of an exemplary Cas9 A homolog of Spy Cas9 in Streptococcus macacae with native 5′-NAAN-3′ PAM specificity is known in the art and described, for example, by Jakimo et al., (www.biorxiv.org/content/biorxiv/early/2018/09/27/429654.full.pdf), and is in the Sequence Listing as SEQ ID NO: 1307.

In some embodiments, a variant Cas9 protein harbors H840A, P475A, W476A, N477A, D1125A, W1126A, and D1218A mutations such that the polypeptide has a reduced ability to cleave a target DNA or RNA. In some embodiments, a variant Cas9 protein harbors H840A, P475A, W476A, N477A, D1125A, W1126A, and D1218A mutations relative to SEQ ID NO: 234. Such a Cas9 protein has a reduced ability to cleave a target DNA (e.g., a single stranded target DNA) but retains the ability to bind a target DNA (e.g., a single stranded target DNA). As another non-limiting example, in some embodiments, the variant Cas9 protein harbors D10A, H840A, P475A, W476A, N477A, D1125A, W1126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 protein harbors D10A, H840A, P475A, W476A, N477A, D1125A, W1126A, and D1218A mutations relative to SEQ ID NO: 234. In some embodiments, when a variant Cas9 protein harbors W476A and W1126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some embodiments, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

Fusion Proteins Comprising a NapDNAbp and a Cytidine Deaminase and/or Adenosine Deaminase

Some aspects of the disclosure provide fusion proteins comprising a Cas9 domain or other nucleic acid programmable DNA binding protein (e.g., Cas12) and one or more cytidine deaminase or adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion protein comprises the following domains A-C, A-D, or A-E:

NH₂-[A-B-C]-COOH;

NH₂-[A-B-C-D]-COOH; or

NH₂-[A-B-C-D-E]-COOH;

wherein A and C or A, C, and E, each comprises one or more of the following:

an adenosine deaminase domain or an active fragment thereof,

a cytidine deaminase domain or an active fragment thereof, and

wherein B or B and D, each comprises one or more domains having nucleic acid sequence specific binding activity.

In some embodiments, the fusion protein comprises the following structure:

NH₂-[Aₙ-Bₒ-Cₙ]-COOH;

NH₂-[Aₙ-Bₒ-Cₙ-Dₒ]-COOH; or

NH₂-[Aₙ-Bₒ-Cₚ-Dₒ-E_{q}]-COOH;

wherein A and C or A, C, and E, each comprises one or more of the following:

an adenosine deaminase domain or an active fragment thereof,

a cytidine deaminase domain or an active fragment thereof, and

wherein n is an integer: 1, 2, 3, 4, or 5, wherein p is an integer: 0, 1, 2, 3, 4, or 5; wherein q is an integer 0, 1, 2, 3, 4, or 5; and wherein B or B and D each comprises a domain having nucleic acid sequence specific binding activity; and wherein o is an integer: 1, 2, 3, 4, or 5.

For example, and without limitation, in some embodiments, the fusion protein comprises the structure:

NH2-[adenosine deaminase]-[Cas9 domain]-COOH;
NH2-[Cas9 domain]-[adenosine deaminase]-COOH;
NH2-[cytidine deaminase]-[Cas9 domain]-COOH;
NH2-[Cas9 domain]-[cytidine deaminase]-COOH;
NH2-[cytidine deaminase]-[Cas9 domain]-[adenosine deaminase]-COOH;
NH2-[adenosine deaminase]-[Cas9 domain]-[cytidine deaminase]-COOH;
NH2-[adenosine deaminase]-[cytidine deaminase]-[Cas9 domain]-COOH;
NH2-[cytidine deaminase]-[adenosine deaminase]-[Cas9 domain]-COOH;
NH2-[Cas9 domain]-[adenosine deaminase]-[cytidine deaminase]-COOH; or
NH2-[Cas9 domain]-[cytidine deaminase]-[adenosine deaminase]-COOH.

In some embodiments, any of the Cas12 domains or Cas12 proteins provided herein may be fused with any of the cytidine or adenosine deaminases provided herein. For example, and without limitation, in some embodiments, the fusion protein comprises the structure:

NH2-[adenosine deaminase]-[Cas12 domain]-COOH;
NH2-[Cas12 domain]-[adenosine deaminase]-COOH;
NH2-[cytidine deaminase]-[Cas12 domain]-COOH;
NH2-[Cas12 domain]-[cytidine deaminase]-COOH;
NH2-[cytidine deaminase]-[Cas12 domain]-[adenosine deaminase]-COOH;
NH2-[adenosine deaminase]-[Cas12 domain]-[cytidine deaminase]-COOH;
NH2-[adenosine deaminase]-[cytidine deaminase]-[Cas12 domain]-COOH;
NH2-[cytidine deaminase]-[adenosine deaminase]-[Cas12 domain]-COOH;
NH2-[Cas12 domain]-[adenosine deaminase]-[cytidine deaminase]-COOH; or
NH2-[Cas12 domain]-[cytidine deaminase]-[adenosine deaminase]-COOH.

In some embodiments, the adenosine deaminase is a TadA*8. Exemplary fusion protein structures include the following:

NH2-[TadA*8]-[Cas9 domain]-COOH;
NH2-[Cas9 domain]-[TadA*8]-COOH;
NH2-[TadA*8]-[Cas12 domain]-COOH; or
NH2-[Cas12 domain][TadA*8]-COOH.

In some embodiments, the adenosine deaminase of the fusion protein comprises a TadA*8 and a cytidine deaminase and/or an adenosine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:

NH2-[TadA*8]-[Cas9/Cas12]-[adenosine deaminase]-COOH;
NH2-[adenosine deaminase]-[Cas9/Cas12]-[TadA*8]-COOH;
NH2-[TadA*8]-[Cas9/Cas12]-[cytidine deaminase]-COOH; or
NH2-[cytidine deaminase]-[Cas9/Cas12]-[TadA*8]-COOH.

In some embodiments, the adenosine deaminase of the fusion protein comprises a TadA*9 and a cytidine deaminase and/or an adenosine deaminase. Exemplary fusion protein structures include the following:

NH2-[TadA*9]-[Cas9/Cas12]-[adenosine deaminase]-COOH;
NH2-[adenosine deaminase]-[Cas9/Cas12]-[TadA*9]-COOH;
NH2-[TadA*9]-[Cas9/Cas12]-[cytidine deaminase]-COOH; or
NH2-[cytidine deaminase]-[Cas9/Cas12]-[TadA*9]-COOH.

In some embodiments, the fusion protein can comprise a deaminase flanked by an N-terminal fragment and a C-terminal fragment of a Cas9 or Cas12 polypeptide. In some embodiments, the fusion protein comprises a cytidine deaminase flanked by an N-terminal fragment and a C-terminal fragment of a Cas9 or Cas12 polypeptide. In some embodiments, the fusion protein comprises an adenosine deaminase flanked by an N-terminal fragment and a C-terminal fragment of a Cas9 or Cas 12 polypeptide.

In some embodiments, the fusion proteins comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, the “-” used in the general architecture above indicates the presence of an optional linker. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Exemplary, yet nonlimiting, fusion proteins are described in International PCT Application Nos. PCT/2017/044935, PCT/US2019/044935, and PCT/US2020/016288, each of which is incorporated herein by reference for its entirety.

### Fusion Proteins Comprising a Nuclear Localization Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence

(SEQ ID NO: 83)
PKKKRKVEGADKRTADGSEFESPKKKRKV,
(SEQ ID NO: 84)
KRTADGSEFESPKKKRKV,
(SEQ ID NO: 85)
KRPAATKKAGQAKKKK,
(SEQ ID NO: 86)
KKTELQTTNAENKTKKL,
(SEQ ID NO: 87)
KRGINDRNFWRGENGRKTR,
(SEQ ID NO: 1424)
RKSGKIAAIWKRPRKPKKKRKV,
or
(SEQ ID NO: 90)
MDSLLMNRRKFLYQFKNVRWAKGRRETYLC.

In some embodiments, the fusion proteins comprising a cytidine or adenosine deaminase, a Cas9 domain, and an NLS do not comprise a linker sequence. In some embodiments, linker sequences between one or more of the domains or proteins (e.g., cytidine or adenosine deaminase, Cas9 domain or NLS) are present. In some embodiments, a linker is present between the cytidine deaminase and adenosine deaminase domains and the napDNAbp. In some embodiments, the “-” used in the general architecture below indicates the presence of an optional linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided herein.

In some embodiments, the general architecture of exemplary napDNAbp (e.g., Cas9 or Cas12) fusion proteins with a cytidine or adenosine deaminase and a napDNAbp (e.g., Cas9 or Cas12) domain comprises any one of the following structures, where NLS is a nuclear localization sequence (e.g., any NLS provided herein), NH₂ is the N-terminus of the fusion protein, and COOH is the C-terminus of the fusion protein:

NH₂—NLS-[cytidine deaminase]-[napDNAbp domain]-COOH;
NH₂—NLS [napDNAbp domain]-[cytidine deaminase]-COOH;
NH₂-[cytidine deaminase]-[napDNAbp domain]-NLS-COOH;
NH₂-[napDNAbp domain]-[cytidine deaminase]-NLS-COOH;
NH₂—NLS-[adenosine deaminase]-[napDNAbp domain]-COOH;
NH₂—NLS [napDNAbp domain]-[adenosine deaminase]-COOH;
NH₂-[adenosine deaminase]-[napDNAbp domain]-NLS-COOH;
NH₂-[napDNAbp domain]-[adenosine deaminase]-NLS-COOH;
NH₂—NLS-[cytidine deaminase]-[napDNAbp domain]-[adenosine deaminase]-COOH;
NH₂—NLS-[adenosine deaminase]-[napDNAbp domain]-[cytidine deaminase]-COOH;
NH₂—NLS-[adenosine deaminase]-[cytidine deaminase]-[napDNAbp domain]-COOH;
NH₂—NLS-[cytidine deaminase]-[adenosine deaminase]-[napDNAbp domain]-COOH;
NH₂—NLS-[napDNAbp domain]-[adenosine deaminase]-[cytidine deaminase]-COOH;
NH₂—NLS-[napDNAbp domain]-[cytidine deaminase]-[adenosine deaminase]-COOH;
NH₂-[cytidine deaminase]-[napDNAbp domain]-[adenosine deaminase]-NLS-COOH;
NH₂-[adenosine deaminase]-[napDNAbp domain]-[cytidine deaminase]-NLS-COOH;
NH₂-[adenosine deaminase]-[cytidine deaminase]-[napDNAbp domain]-NLS-COOH;
NH₂-[cytidine deaminase]-[adenosine deaminase]-[napDNAbp domain]-NLS-COOH;
NH₂-[napDNAbp domain]-[adenosine deaminase]-[cytidine deaminase]-NLS-COOH; or

NH₂-[napDNAbp domain]-[cytidine deaminase]-[adenosine deaminase]-NLS-COOH. In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK (SEQ ID NO: 85), is the prototype of the ubiquitous bipartite signal: two clusters of basic amino acids, separated by a spacer of about 10 amino acids. The sequence of an exemplary bipartite NLS follows:

(SEQ ID NO: 83)
PKKKRKVEGADKRTADGSEFESPKKKRKV

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the amino-terminus, about or more than about 1, 2, 3, 4, 5, 6, 7, 8, 9, 10 NLSs at or near the carboxy-terminus, or any combination thereof (e.g., one or more NLS at the amino-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor can comprise a nuclease, a nickase, a recombinase, a deaminase, a methyltransferase, a methylase, an acetylase, an acetyltransferase, a transcriptional activator, or a transcriptional repressor domain.

In some embodiments, a base editor can comprise an uracil glycosylase inhibitor (UGI) domain. In some embodiments, cellular DNA repair response to the presence of U: G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein comprising a UGI domain.

In some embodiments, a base editor comprises as a domain all or a portion of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitutions in any domain does not change the length of the base editor.

Non-limiting examples of such base editors, where the length of all the domains is the same as the wild type domains, can include:

NH2-[nucleobase editing domain]-Linker1-[APOBEC1]-Linker2-[nucleobase editing domain]-COOH;
NH2-[nucleobase editing domain]-Linker1-[APOBEC1]-[nucleobase editing domain]-COOH;
NH2-[nucleobase editing domain]-[APOBEC1]-Linker2-[nucleobase editing domain]-COOH;
NH2-[nucleobase editing domain]-[APOBEC1]-[nucleobase editing domain]-COOH;
NH2-[nucleobase editing domain]-Linker1-[APOBEC1]-Linker2-[nucleobase editing domain]-[UGI]-COOH;
NH2-[nucleobase editing domain]-Linker1-[APOBEC1]-[nucleobase editing domain]-[UGI]-COOH;
NH2-[nucleobase editing domain]-[APOBEC1]-Linker2-[nucleobase editing domain]-[UGI]-COOH;
NH2-[nucleobase editing domain]-[APOBEC1]-[nucleobase editing domain]-[UGI]-COOH;
NH2-[UGI]-[nucleobase editing domain]-Linker1-[APOBEC1]-Linker2-[nucleobase editing domain]-COOH;
NH2-[UGI]-[nucleobase editing domain]-Linker1-[APOBEC1]-[nucleobase editing domain]-COOH;
NH2-[UGI]-[nucleobase editing domain]-[APOBEC1]-Linker2-[nucleobase editing domain]-COOH; or
NH2-[UGI]-[nucleobase editing domain]-[APOBEC1]-[nucleobase editing domain]-COOH.

### F. BASE EDITOR SYSTEM

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

In some embodiments, a base editing system as provided herein provides a new approach to genome editing that uses a fusion protein containing a catalytically defective Streptococcus pyogenes Cas9, a deaminase (e.g., cytidine or adenosine deaminase), and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleic acid (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

In some embodiments, a single guide polynucleotide may be utilized to target a deaminase to a target nucleic acid sequence. In some embodiments, a single pair of guide polynucleotides may be utilized to target different deaminases to a target nucleic acid sequence.

The nucleobase components and the polynucleotide programmable nucleotide binding component of a base editor system may be associated with each other covalently or non-covalently. For example, in some embodiments, the deaminase domain can be targeted to a target nucleotide sequence by a polynucleotide programmable nucleotide binding domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Corn coat protein domain, a steril alpha motif, a telomerase Ku binding motif and Ku protein, a telomerase Sm7 binding motif and Sm7 protein, or an RNA recognition motif.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system, e.g., the deaminase component, can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Corn coat protein domain, a sterile alpha motif, a telomerase Ku binding motif and Ku protein, a telomerase Sm7 binding motif and Sm7 protein, or an RNA recognition motif.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain. In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component can comprise an additional heterologous portion or domain that is capable of interacting with, associating with, or capable of forming a complex with an additional heterologous portion or domain that is part of a polynucleotide programmable nucleotide binding domain. In some embodiments, the inhibitor of base excision repair can be targeted to the target nucleotide sequence by the guide polynucleotide. For example, in some embodiments, the inhibitor of base excision repair can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Corn coat protein domain, a sterile alpha motif, a telomerase Ku binding motif and Ku protein, a telomerase Sm7 binding motif and Sm7 protein, or an RNA recognition motif.

In some embodiments, the base editor inhibits base excision repair (BER) of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the base editing fusion proteins provided herein need to be positioned at a precise location, for example, where a target base is placed within a defined region (e.g., a “deamination window”). In some embodiments, a target can be within a 4 base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n(D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂ (SEQ ID NO: 1425)-XTEN-(SGGS)₂ (“(SGGS)₂” disclosed as SEQ ID NO: 1425)) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild-type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and I156F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in Table 10 below. In some embodiments, the ABE is a sixth generation ABE. In some embodiments, the ABE is ABE6.1, ABE6.2, ABE6.3, ABE6.4, ABE6.5, or ABE6.6, as shown in Table 10 below. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE 7.9, or ABE7.10, as shown in Table 10 below.

TABLE 10
Genotypes of ABEs

| | 26 | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|---|
| ABE0.1 | R | W | R | H | N | P | | R | N | L | S | A | D | H | G | A | S | D | R | E | I | K | K |
| ABE0.2 | R | W | R | H | N | P | | R | N | L | S | A | D | H | G | A | S | D | R | E | I | K | K |
| ABE1.1 | R | W | R | H | N | P | | R | N | L | S | A | N | H | G | A | S | D | R | E | I | K | K |
| ABE1.2 | R | W | R | H | N | P | | R | N | L | S | V | N | H | G | A | S | D | R | E | I | K | K |
| ABE2.1 | R | W | R | H | N | P | | R | N | L | S | V | N | H | G | A | S | Y | R | V | I | K | K |
| ABE2.2 | R | W | R | H | N | P | | R | N | L | S | V | N | H | G | A | S | Y | R | V | I | K | K |
| ABE2.3 | R | W | R | H | N | P | | R | N | L | S | V | N | H | G | A | S | Y | R | V | I | K | K |
| ABE2.4 | R | W | R | H | N | P | | R | N | L | S | V | N | H | G | A | S | Y | R | V | I | K | K |
| ABE2.5 | R | W | R | H | N | P | | R | N | L | S | V | N | H | G | A | S | Y | R | V | I | K | K |
| ABE2.6 | R | W | R | H | N | P | | R | N | L | S | V | N | H | G | A | S | Y | R | V | I | K | K |
| ABE2.7 | R | W | R | H | N | P | | R | N | L | S | V | N | H | G | A | S | Y | R | V | I | K | K |
| ABE2.8 | R | W | R | H | N | P | | R | N | L | S | V | N | H | G | A | S | Y | R | V | I | K | K |
| ABE2.9 | R | W | R | H | N | P | | R | N | L | S | V | N | H | G | A | S | Y | R | V | I | K | K |
| ABE2.10 | R | W | R | H | N | P | | R | N | L | S | V | N | H | G | A | S | Y | R | V | I | K | K |
| ABE2.11 | R | W | R | H | N | P | | R | N | L | S | V | N | H | G | A | S | Y | R | V | I | K | K |
| ABE2.12 | R | W | R | H | N | P | | R | N | L | S | V | N | H | G | A | S | Y | R | V | I | K | K |
| ABE3.1 | R | W | R | H | N | P | | R | N | F | S | V | N | Y | G | A | S | Y | R | V | F | K | K |
| ABE3.2 | R | W | R | H | N | P | | R | N | F | S | V | N | Y | G | A | S | Y | R | V | F | K | K |
| ABE3.3 | R | W | R | H | N | P | | R | N | F | S | V | N | Y | G | A | S | Y | R | V | F | K | K |
| ABE3.4 | R | W | R | H | N | P | | R | N | F | S | V | N | Y | G | A | S | Y | R | V | F | K | K |
| ABE3.5 | R | W | R | H | N | P | | R | N | F | S | V | N | Y | G | A | S | Y | R | V | F | K | K |
| ABE3.6 | R | W | R | H | N | P | | R | N | F | S | V | N | Y | G | A | S | Y | R | V | F | K | K |
| ABE3.7 | R | W | R | H | N | P | | R | N | F | S | V | N | Y | G | A | S | Y | R | V | F | K | K |
| ABE3.8 | R | W | R | H | N | P | | R | N | F | S | V | N | Y | G | A | S | Y | R | V | F | K | K |
| ABE4.1 | R | W | R | H | N | P | | R | N | L | S | V | N | H | G | N | S | Y | R | V | I | K | K |
| ABE4.2 | G | W | G | H | N | P | | R | N | L | S | V | N | H | G | N | S | Y | R | V | I | K | K |
| ABE4.3 | R | W | R | H | N | P | | R | N | F | S | V | N | Y | G | N | S | Y | R | V | F | K | K |
| ABE5.1 | R | W | R | L | N | P | | L | N | F | S | V | N | Y | G | A | C | Y | R | V | F | N | K |
| ABE5.2 | R | W | R | H | S | P | | R | N | F | S | V | N | Y | G | A | S | Y | R | V | F | K | T |
| ABE5.3 | R | W | R | L | N | P | | L | N | I | S | V | N | Y | G | A | C | Y | R | V | F | N | K |
| ABE5.4 | R | W | R | H | S | P | | R | N | F | S | V | N | Y | G | A | S | Y | R | V | F | K | T |
| ABE5.5 | R | W | R | L | N | P | | L | N | F | S | V | N | Y | G | A | C | Y | R | V | F | N | K |
| ABE5.6 | R | W | R | L | N | P | | L | N | F | S | V | N | Y | G | A | C | Y | R | V | F | N | K |
| ABE5.7 | R | W | R | L | N | P | | L | N | F | S | V | N | Y | G | A | C | Y | R | V | F | N | K |
| ABE5.8 | R | W | R | L | N | P | | L | N | F | S | V | N | Y | G | A | C | Y | R | V | F | N | K |
| ABE5.9 | R | W | R | L | N | P | | L | N | F | S | V | N | Y | G | A | C | Y | R | V | F | N | K |
| ABE5.10 | R | W | R | L | N | P | | L | N | F | S | V | N | Y | G | A | C | Y | R | V | F | N | K |
| ABE5.11 | R | W | R | L | N | P | | L | N | F | S | V | N | Y | G | A | C | Y | R | V | F | N | K |
| ABE5.12 | R | W | R | L | N | P | | L | N | F | S | V | N | Y | G | A | C | Y | R | V | F | N | K |
| ABE5.13 | R | W | R | H | N | P | | L | D | F | S | V | N | Y | A | A | S | Y | R | V | F | K | K |
| ABE5.14 | R | W | R | H | N | S | | L | N | F | C | V | N | Y | G | A | S | Y | R | V | F | K | K |
| ABE6.1 | R | W | R | H | N | S | | L | N | F | S | V | N | Y | G | N | S | Y | R | V | F | K | K |
| ABE6.2 | R | W | R | H | N | T | V | L | N | F | S | V | N | Y | G | N | S | Y | R | V | F | N | K |
| ABE6.3 | R | W | R | L | N | S | | L | N | F | S | V | N | Y | G | A | C | Y | R | V | F | N | K |
| ABE6.4 | R | W | R | L | N | S | | L | N | F | S | V | N | Y | G | N | C | Y | R | V | F | N | K |
| ABE6.5 | R | W | R | L | N | T | V | L | N | F | S | V | N | Y | G | A | C | Y | R | V | F | N | K |
| ABE6.6 | R | W | R | L | N | T | V | L | N | F | S | V | N | Y | G | N | C | Y | R | V | F | N | K |
| ABE7.1 | R | W | R | L | N | A | | L | N | F | S | V | N | Y | G | A | C | Y | R | V | F | N | K |
| ABE7.2 | R | W | R | L | N | A | | L | N | F | S | V | N | Y | G | N | C | Y | R | V | F | N | K |
| ABE7.3 | R | L | R | L | N | A | | L | N | F | S | V | N | Y | G | A | C | Y | R | V | F | N | K |
| ABE7.4 | R | R | R | L | N | A | | L | N | F | S | V | N | Y | G | A | C | Y | R | V | F | N | K |
| ABE7.5 | R | W | R | L | N | A | | L | N | F | S | V | N | Y | G | A | C | Y | H | V | F | N | K |
| ABE7.6 | R | W | R | L | N | A | | L | N | I | S | V | N | Y | G | A | C | Y | P | V | F | N | K |
| ABE7.7 | R | L | R | L | N | A | | L | N | F | S | V | N | Y | G | A | C | Y | P | V | F | N | K |
| ABE7.8 | R | L | R | L | N | A | | L | N | F | S | V | N | Y | G | N | C | Y | R | V | F | N | K |
| ABE7.9 | R | L | R | L | N | A | | L | N | F | S | V | N | Y | G | N | C | Y | P | V | F | N | K |
| ABE7.10 | R | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12).

In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24).

In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 11 below.

TABLE 11
Adenosine Deaminase Base Editor 8 (ABE8) Variants

| | Adenosine | |
|---|---|---|
| ABE8 | Deaminase | Adenosine Deaminase Description |
| ABE8.1-m | TadA*8.1 | Monomer_TadA*7.10 + Y147T |
| ABE8.2-m | TadA*8.2 | Monomer_TadA*7.10 + Y147R |
| ABE8.3-m | TadA*8.3 | Monomer_TadA*7.10 + Q154S |
| ABE8.4-m | TadA*8.4 | Monomer_TadA*7.10 + Y123H |
| ABE8.5-m | TadA*8.5 | Monomer_TadA*7.10 + V82S |
| ABE8.6-m | TadA*8.6 | Monomer_TadA*7.10 + T166R |
| ABE8.7-m | TadA*8.7 | Monomer_TadA*7.10 + Q154R |
| ABE8.8-m | TadA*8.8 | Monomer_TadA*7.10 + Y147R_Q154R_Y123H |
| ABE8.9-m | TadA*8.9 | Monomer_TadA*7.10 + Y147R_Q154R_I76Y |
| ABE8.10-m | TadA*8.10 | Monomer_TadA*7.10 + Y147R_Q154R_T166R |
| ABE8.11-m | TadA*8.11 | Monomer_TadA*7.10 + Y147T_Q154R |
| ABE8.12-m | TadA*8.12 | Monomer_TadA*7.10 + Y147T_Q154S |
| ABE8.13-m | TadA*8.13 | Monomer_TadA*7.10 + Y123H_Y147R_Q154R_I76Y |
| ABE8.14-m | TadA*8.14 | Monomer_TadA*7.10 + I76Y_V82S |
| ABE8.15-m | TadA*8.15 | Monomer_TadA*7.10 + V82S_Y147R |
| ABE8.16-m | TadA*8.16 | Monomer_TadA*7.10 + V82S_Y123H_Y147R |
| ABE8.17-m | TadA*8.17 | Monomer_TadA*7.10 + V82S_Q154R |
| ABE8.18-m | TadA*8.18 | Monomer_TadA*7.10 + V82S_Y123H_Q154R |
| ABE8.19-m | TadA*8.19 | Monomer_TadA*7.10 + V82S_Y123H_Y147R_Q154R |
| ABE8.20-m | TadA*8.20 | Monomer TadA*7.10 + |
| | | I76Y_V82S_Y123H_Y147R_Q154R |
| ABE8.21-m | TadA*8.21 | Monomer_TadA*7.10 + Y147R_Q154S |
| ABE8.22-m | TadA*8.22 | Monomer_TadA*7.10 + V82S_Q154S |
| ABE8.23-m | TadA*8.23 | Monomer_TadA*7.10 + V82S_Y123H |
| ABE8.24-m | TadA*8.24 | Monomer_TadA*7.10 + V82S_Y123H_Y147T |
| ABE8.1-d | TadA*8.1 | Heterodimer_(WT) + (TadA*7.10 + Y147T) |
| ABE8.2-d | TadA*8.2 | Heterodimer_(WT) + (TadA*7.10 + Y147R) |
| ABE8.3-d | TadA*8.3 | Heterodimer_(WT) + (TadA*7.10 + Q154S) |
| ABE8.4-d | TadA*8.4 | Heterodimer_(WT) + (TadA*7.10 + Y123H) |
| ABE8.5-d | TadA*8.5 | Heterodimer_(WT) + (TadA*7.10 + V82S) |
| ABE8.6-d | TadA*8.6 | Heterodimer_(WT) + (TadA*7.10 + T166R) |
| ABE8.7-d | TadA*8.7 | Heterodimer_(WT) + (TadA*7.10 + Q154R) |
| ABE8.8-d | TadA*8.8 | Heterodimer_(WT) + (TadA*7.10 + |
| | | Y147R_Q154R_Y123H) |
| ABE8.9-d | TadA*8.9 | Heterodimer_(WT) + (TadA*7.10 + |
| | | Y147R_Q154R_I76Y) |
| ABE8.10-d | TadA*8.10 | Heterodimer_(WT) + (TadA*7.10 + |
| | | Y147R_Q154R_T166R) |
| ABE8.11-d | TadA*8.11 | Heterodimer_(WT) + (TadA*7.10 + Y147T_Q154R) |
| ABE8.12-d | TadA*8.12 | Heterodimer_(WT) + (TadA*7.10 + Y147T_Q154S) |
| ABE8.13-d | TadA*8.13 | Heterodimer_(WT) + (TadA*7.10 + |
| | | Y123H_Y147T_Q154R_I76Y) |
| ABE8.14-d | TadA*8.14 | Heterodimer_(WT) + (TadA*7.10 + I76Y_V82S) |
| ABE8.15-d | TadA*8.15 | Heterodimer_(WT) + (TadA*7.10 + V82S_ Y147R) |
| ABE8.16-d | TadA*8.16 | Heterodimer_(WT) + (TadA*7.10 + |
| | | V82S_Y123H_Y147R) |
| ABE8.17-d | TadA*8.17 | Heterodimer_(WT) + (TadA*7.10 + V82S_Q154R) |
| ABE8.18-d | TadA*8.18 | Heterodimer_(WT) + (TadA*7.10 + |
| | | V82S_Y123H_Q154R) |
| ABE8.19-d | TadA*8.19 | Heterodimer_(WT) + (TadA*7.10 + |
| | | V82S_Y123H_Y147R_Q154R) |
| ABE8.20-d | TadA*8.20 | Heterodimer_(WT) + (TadA*7.10 + |
| | | I76Y_V82S_Y123H_Y147R_Q154R) |
| ABE8.21-d | TadA*8.21 | Heterodimer_(WT) + (TadA*7.10 + Y147R_Q154S) |
| ABE8.22-d | TadA*8.22 | Heterodimer_(WT) + (TadA*7.10 + V82S_Q154S) |
| ABE8.23-d | TadA*8.23 | Heterodimer_(WT) + (TadA*7.10 + V82S_Y123H) |
| ABE8.24-d | TadA*8.24 | Heterodimer_(WT) + (TadA*7.10 + |
| | | V82S_Y123H_Y147T) |

In some embodiments, the ABE8 is ABE8a-m, which has a monomeric construct containing TadA*7.10 with R26C, A109S, T111R, D119N, H122N, Y147D, F149Y, T166I, and D167N mutations (TadA*8a). In some embodiments, the ABE8 is ABE8b-m, which has a monomeric construct containing TadA*7.10 with V88A, A109S, T111R, D119N, H122N, F149Y, T166I, and D167N mutations (TadA*8b). In some embodiments, the ABE8 is ABE8c-m, which has a monomeric construct containing TadA*7.10 with R26C, A109S, T111R, D119N, H122N, F149Y, T166I, and D167N mutations (TadA*8c). In some embodiments, the ABE8 is ABE8d-m, which has a monomeric construct containing TadA*7.10 with V88A, T111R, D119N, and F149Y mutations (TadA*8d). In some embodiments, the ABE8 is ABE8e-m, which has a monomeric construct containing TadA*7.10 with A109S, T111R, D119N, H122N, Y147D, F149Y, T166I, and D167N mutations (TadA*8e).

In some embodiments, the ABE8 is ABE8a-d, which has a heterodimeric construct containing wild-type E. coli TadA fused to TadA*7.10 with R26C, A109S, T111R, D119, H122N, Y147D, F149Y, T166I, and D167N mutations (TadA*8a). In some embodiments, the ABE8 is ABE8b-d, which has a heterodimeric construct containing wild-type E. coli TadA fused to TadA*7.10 with V88A, A109S, T111R, D119N, H122N, F149Y, T166I, and D167N mutations (TadA*8b). In some embodiments, the ABE8 is ABE8c-d, which has a heterodimeric construct containing wild-type E. coli TadA fused to TadA*7.10 with R26C, A109S, T111R, D119N, H122N, F149Y, T166I, and D167N mutations (TadA*8c). In some embodiments, the ABE8 is ABE8d-d, which has a heterodimeric construct containing wild-type E. coli TadA fused to TadA*7.10 with V88A, T111R, D119N, and F149Y mutations (TadA*8d). In some embodiments, the ABE8 is ABE8e-d, which has a heterodimeric construct containing wild-type E. coli TadA fused to TadA*7.10 with A109S, T111R, D119N, H122N, Y147D, F149Y, T166I, and D167N mutations (TadA*8e).

In some embodiments, the ABE8 is ABE8a-7, which has a heterodimeric construct containing TadA*7.10 fused to TadA*7.10 with R26C, A109S, T111R, D119, H122N, Y147D, F149Y, T166I, and D167N mutations (TadA*8a). In some embodiments, the ABE8 is ABE8b-7, which has a heterodimeric construct containing TadA*7.10 fused to TadA*7.10 with V88A, A109S, T111R, D119N, H122N, F149Y, T166I, and D167N mutations (TadA*8b). In some embodiments, the ABE8 is ABE8c-7, which has a heterodimeric construct containing TadA*7.10 fused to TadA*7.10 with R26C, A109S, T111R, D119N, H122N, F149Y, T166I, and D167N mutations (TadA*8c). In some embodiments, the ABE8 is ABE8d-7, which has a heterodimeric construct containing TadA*7.10 fused to TadA*7.10 with V88A, T111R, D119N, and F149Y mutations (TadA*8d). In some embodiments, the ABE8 is ABE8e-7, which has a heterodimeric construct containing TadA*7.10 fused to TadA*7.10 with A109S, T111R, D119N, H122N, Y147D, F149Y, T166I, and D167N mutations (TadA*8e).

In some embodiments, the ABE is ABE8a-m, ABE8b-m, ABE8c-m, ABE8d-m, ABE8e-m, ABE8a-d, ABE8b-d, ABE8c-d, ABE8d-d, or ABE8e-d, as shown in Table 12 below. In some embodiments, the ABE is ABE8e-m or ABE8e-d. ABE8e shows efficient adenine base editing activity and low indel formation when used with Cas homologues other than SpCas9, for example, SaCas9, SaCas9-KKH, Cas12a homologues, e.g., LbCas12a, enAs-Cas12a, SpCas9-NG and circularly permuted CP1028-SpCas9 and CP1041-SpCas9. In addition to the mutations shown for ABE8e in Table 12, off-target RNA and DNA editing were reduced by introducing a V106W substitution into the TadA domain (as described in M. Richter et al., 2020, Nature Biotechnology, doi.org/10.1038/s41587-020-0453-z, the entire contents of which are incorporated by reference herein).

TABLE 12
Additional Adenosine Deaminase Base Editor 8 Variants. In the table, “monomer”
indicates an ABE comprising a single TadA*7.10 comprising the indicated alterations
and “heterodimer” indicates an ABE comprising a TadA*7.10 comprising the
indicated alterations fused to an E. coli TadA adenosine deaminase.

| ABE8 Base | Adenosine | |
|---|---|---|
| Editor | Deaminase | Adenosine Deaminase Description |
| ABE8a-m | TadA*8a | Monomer_TadA*7.10 + R26C + A109S + T111R + D119N + |
| | | H122N + Y147D + F149Y + T166I + D167N |
| ABE8b-m | TadA*8b | Monomer_TadA*7.10 + V88A + A109S + T111R + D119N + |
| | | H122N + F149Y + T166I + D167N |
| ABE8c-m | TadA*8c | Monomer_TadA*7.10 + R26C + A109S + T111R + D119N + |
| | | H122N + F149Y + T166I + D167N |
| ABE8d-m | TadA*8d | Monomer_TadA*7.10 + V88A + T111R + D119N + F149Y |
| ABE8e-m | TadA*8e | Monomer_TadA*7.10 + A109S + T111R + D119N + H122N + |
| | | Y147D + F149Y + T166I + D167N |
| ABE8a-d | TadA*8a | Heterodimer_(WT) + (TadA*7.10 + R26C + A109S + T111R + |
| | | D119N + H122N + Y147D + F149Y + T166I + D167N) |
| ABE8b-d | TadA*8b | Heterodimer_(WT) + (TadA*7.10 + V88A + A109S + T111R + |
| | | D119N + H122N + F149Y + T166I + D167N) |
| ABE8c-d | TadA*8c | Heterodimer_(WT) + (TadA*7.10 + R26C + A109S + T111R + |
| | | D119N + H122N + F149Y + T166I + D167N) |
| ABE8d-d | TadA*8d | Heterodimer_(WT) + (TadA*7.10 + V88A + T111R + D119N + |
| | | F149Y) |
| ABE8e-d | TadA*8e | Heterodimer_(WT) + (TadA*7.10 + A109S + T111R + D119N + |
| | | H122N + Y147D + F149Y + T166I + D167N) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyogenes Cas9 or spVRQR Cas9). In some embodiments, the base editor (e.g., ABE7.9, ABE7.10, or ABE8) is an AGA PAM CP5 variant (S. pyogenes Cas9 or spVRQR Cas9). In some embodiments, the base editor (e.g., ABE7.9, ABE7.10, or ABE8) is an NGC PAM CP6 variant (S. pyogenes Cas9 or spVRQR Cas9). In some embodiments, the base editor (e.g. ABE7.9, ABE7.10, or ABE8) is an AGA PAM CP6 variant (S. pyogenes Cas9 or spVRQR Cas9).

In some embodiments, the ABE has a genotype as shown in Table 13 below.

TABLE 13
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 14 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 14 below.

Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | | | S | | | | |
| ABE8.4-m | | | | | | | | | | H | | | | | | | | |
| ABE8.5-m | | | | | | S | | | | | | | | | | | | |
| ABE8.6-m | | | | | | | | | | | | | | | | | | R |
| ABE8.7-m | | | | | | | | | | | | | | R | | | | |
| ABE8.8-m | | | | | | | | | | H | | R | | R | | | | |
| ABE8.9-m | | | | | Y | | | | | | | R | | R | | | | |
| ABE8.10- | | | | | | | | | | | | R | | R | | | | R |
| m | | | | | | | | | | | | | | | | | | |
| ABE8.11- | | | | | | | | | | | | T | | R | | | | |
| m | | | | | | | | | | | | | | | | | | |
| ABE8.12- | | | | | | | | | | | | T | | S | | | | |
| m | | | | | | | | | | | | | | | | | | |
| ABE8.13- | | | | | Y | | | | | H | | R | | R | | | | |
| m | | | | | | | | | | | | | | | | | | |
| ABE8.14- | | | | | Y | S | | | | | | | | | | | | |
| m | | | | | | | | | | | | | | | | | | |
| ABE8.15- | | | | | | S | | | | | | R | | | | | | |
| m | | | | | | | | | | | | | | | | | | |
| ABE8.16- | | | | | | S | | | | H | | R | | | | | | |
| m | | | | | | | | | | | | | | | | | | |
| ABE8.17- | | | | | | S | | | | | | | | R | | | | |
| m | | | | | | | | | | | | | | | | | | |
| ABE8.18- | | | | | | S | | | | H | | | | R | | | | |
| m | | | | | | | | | | | | | | | | | | |
| ABE8.19- | | | | | | S | | | | H | | R | | R | | | | |
| m | | | | | | | | | | | | | | | | | | |
| ABE8.20- | | | | | Y | S | | | | H | | R | | R | | | | |
| m | | | | | | | | | | | | | | | | | | |
| ABE8.21- | | | | | | | | | | | | R | | S | | | | |
| m | | | | | | | | | | | | | | | | | | |
| ABE8.22- | | | | | | S | | | | | | | | S | | | | |
| m | | | | | | | | | | | | | | | | | | |
| ABE8.23- | | | | | | S | | | | H | | | | | | | | |
| m | | | | | | | | | | | | | | | | | | |
| ABE8.24- | | | | | | S | | | | H | | T | | | | | | |
| m | | | | | | | | | | | | | | | | | | |
| ABE8.1-d | | | | | | | | | | | | T | | | | | | |
| ABE8.2-d | | | | | | | | | | | | R | | | | | | |
| ABE8.3-d | | | | | | | | | | | | | | S | | | | |
| ABE8.4-d | | | | | | | | | | H | | | | | | | | |
| ABE8.5-d | | | | | | S | | | | | | | | | | | | |
| ABE8.6-d | | | | | | | | | | | | | | | | | | R |
| ABE8.7-d | | | | | | | | | | | | | | R | | | | |
| ABE8.8-d | | | | | | | | | | H | | R | | R | | | | |
| ABE8.9-d | | | | | Y | | | | | | | R | | R | | | | |
| ABE8.10-d | | | | | | | | | | | | R | | R | | | | R |
| ABE8.11-d | | | | | | | | | | | | T | | R | | | | |
| ABE8.12-d | | | | | | | | | | | | T | | S | | | | |
| ABE8.13-d | | | | | Y | | | | | H | | R | | R | | | | |
| ABE8.14-d | | | | | Y | S | | | | | | | | | | | | |
| ABE8.15-d | | | | | | S | | | | | | R | | | | | | |
| ABE8.16-d | | | | | | S | | | | H | | R | | | | | | |
| ABE8.17-d | | | | | | S | | | | | | | | R | | | | |
| ABE8.18-d | | | | | | S | | | | H | | | | R | | | | |
| ABE8.19-d | | | | | | S | | | | H | | R | | R | | | | |
| ABE8.20-d | | | | | Y | S | | | | H | | R | | R | | | | |
| ABE8.21d | | | | | | | | | | | | R | | S | | | | |
| ABE8.22-d | | | | | | S | | | | | | | | S | | | | |
| ABE8.23-d | | | | | | S | | | | H | | | | | | | | |
| ABE8.24-d | | | | | | S | | | | H | | T | | | | | | |

In some embodiments, the base editor is ABE8.1, which comprises or consists essentially of the following sequence or a fragment thereof having adenosine deaminase activity:

ABE8.1_Y147T_CP5_NGC PAM_monomer
(SEQ ID NO: 1426)
MSEVEFSHEYWMRHALTLAKRARDEREVPVGAVLV
LNNRVIGEGWNRAIGLHDPTAHAEIMALRQGGLVM
QNYRLIDATLYVTFEPCVMCAGAMIHSRIGRVVFG
VRNAKTGAAGSLMDVLHYPGMNHRVEITEGILADE
CAALLCTFFRMPRQVFNAQKKAQSSTD

[symbol]

[symbol]

EIGKATAKYFFY
SNIMNFFKTEITLANGEIRKRPLIETNGETGEIVW
DKGRDFATVRKVLSMPQVNIVKKTEVQTGGFSKES
ILPKRNSDKLIARKKDWDPKKYGGFMQPTVAYSVL
VVAKVEKGKSKKLKSVKELLGITIMERSSFEKNPI
DFLEAKGYKEVKKDLIIKLPKYSLFELENGRKRML
ASAKFLQKGNELALPSKYVNFLYLASHYEKLKGSP
EDNEQKQLFVEQHKHYLDEIIEQISEFSKRVILAD
ANLDKVLSAYNKHRDKPIREQAENIIHLFTLTNLG
APRAFKYFDTTIARKEYRSTKEVLDATLIHQSITG
LYETRIDLSQLGGD [symbol] [symbol]
DKKYSIGLAIGTNSVGWAVITDEYKVPSKKFKVLG
NTDRHSIKKNLIGALLFDSGETAEATRLKRTARRR
YTRRKNRICYLQEIFSNEMAKVDDSFFHRLEESFL
VEEDKKHERHPIFGNIVDEVAYHEKYPTIYHLRKK
LVDSTDKADLRLIYLALAHMIKFRGHFLIEGDLNP
DNSDVDKLFIQLVQTYNQLFEENPINASGVDAKAI
LSARLSKSRRLENLIAQLPGEKKNGLFGNLIALSL
GLTPNFKSNFDLAEDAKLQLSKDTYDDDLDNLLAQ
IGDQYADLFLAAKNLSDAILLSDILRVNTEITKAP
LSASMIKRYDEHHQDLTLLKALVRQQLPEKYKEIF
FDQSKNGYAGYIDGGASQEEFYKFIKPILEKMDGT
EELLVKLNREDLLRKQRTFDNGSIPHQIHLGELHA
ILRRQEDFYPFLKDNREKIEKILTFRIPYYVGPLA
RGNSRFAWMTRKSEETITPWNFEEVVDKGASAQSF
IERMTNFDKNLPNEKVLPKHSLLYEYFTVYNELTK
VKYVTEGMRKPAFLSGEQKKAIVDLLFKTNRKVTV
KQLKEDYFKKIECFDSVEISGVEDRFNASLGTYHD
LLKIIKDKDFLDNEENEDILEDIVLTLTLFEDREM
IEERLKTYAHLFDDKVMKQLKRRRYTGWGRLSRKL
INGIRDKQSGKTILDFLKSDGFANRNFMQLIHDDS
LTFKEDIQKAQVSGQGDSLHEHIANLAGSPAIKKG
ILQTVKVVDELVKVMGRHKPENIVIEMARENQTTQ
KGQKNSRERMKRIEEGIKELGSQILKEHPVENTQL
QNEKLYLYYLQNGRDMYVDQELDINRLSDYDVDHI
VPQSFLKDDSIDNKVLTRSDKNRGKSDNVPSEEVV
KKMKNYWRQLLNAKLITQRKFDNLTKAERGGLSEL
DKAGFIKRQLVETRQITKHVAQILDSRMNTKYDEN
DKLIREVKVITLKSKLVSDFRKDFQFYKVREINNY
HHAHDAYLNAVVGTALIKKYPKLESEFVYGDYKVY
DVRKMIAKSEQEGADKRTADGSEFESPKKKRKV

In the above sequence, the plain text denotes an adenosine deaminase sequence, bold sequence indicates sequence derived from Cas9, the italicized sequence denotes a linker sequence, and the underlined sequence denotes a bipartite nuclear localization sequence. Other ABE8 sequences are provided in the attached sequence listing (SEQ ID NOs: 1427-1449).

In some embodiments, the base editor is a ninth generation ABE (ABE9). In some embodiments, the ABE9 contains a TadA*9 variant. ABE9 base editors include an adenosine deaminase variant comprising an amino acid sequence, which contains alterations relative to an ABE 7*10 reference sequence, as described herein. Exemplary ABE9 variants are listed in Table 15. Details of ABE9 base editors are described in International PCT Application No. PCT/2020/049975, which is incorporated herein by reference for its entirety.

TABLE 15
Adenosine Deaminase Base Editor 9 (ABE9) Variants. In the table, “monomer”
indicates an ABE comprising a single TadA*7.10 comprising the indicated alterations
and “heterodimer” indicates an ABE comprising a TadA*7.10 comprising the
indicated alterations fused to an E. coli TadA adenosine deaminase.

| ABE9 Description | Alterations |
|---|---|
| ABE9.1_monomer | E25F, V82S, Y123H, T133K, Y147R, Q154R |
| ABE9.2_monomer | E25F, V82S, Y123H, Y147R, Q154R |
| ABE9.3_monomer | V82S, Y123H, P124W, Y147R, Q154R |
| ABE9.4_monomer | L51W, V82S, Y123H, C146R, Y147R, Q154R |
| ABE9.5_monomer | P54C, V82S, Y123H, Y147R, Q154R |
| ABE9.6_monomer | Y73S, V82S, Y123H, Y147R, Q154R |
| ABE9.7_monomer | N38G, V82T, Y123H, Y147R, Q154R |
| ABE9.8_monomer | R23H, V82S, Y123H, Y147R, Q154R |
| ABE9.9_monomer | R21N, V82S, Y123H, Y147R, Q154R |
| ABE9.10_monomer | V82S, Y123H, Y147R, Q154R, A158K |
| ABE9.11_monomer | N72K, V82S, Y123H, D139L, Y147R, Q154R, |
| ABE9.12_monomer | E25F, V82S, Y123H, D139M, Y147R, Q154R |
| ABE9.13_monomer | M70V, V82S, M94V, Y123H, Y147R, Q154R |
| ABE9.14_monomer | Q71M, V82S, Y123H, Y147R, Q154R |
| ABE9.15_heterodimer | E25F, V82S, Y123H, T133K, Y147R, Q154R |
| ABE9.16_heterodimer | E25F, V82S, Y123H, Y147R, Q154R |
| ABE9.17_heterodimer | V82S, Y123H, P124W, Y147R, Q154R |
| ABE9.18_heterodimer | L51W, V82S, Y123H, C146R, Y147R, Q154R |
| ABE9.19_heterodimer | P54C, V82S, Y123H, Y147R, Q154R |
| ABE9.2 _heterodimer | Y73S, V82S, Y123H, Y147R, Q154R |
| ABE9.21_heterodimer | N38G, V82T, Y123H, Y147R, Q154R |
| ABE9.22_heterodimer | R23H, V82S, Y123H, Y147R, Q154R |
| ABE9.23_heterodimer | R21N, V82S, Y123H, Y147R, Q154R |
| ABE9.24_heterodimer | V82S, Y123H, Y147R, Q154R, A158K |
| ABE9.25_heterodimer | N72K, V82S, Y123H, D139L, Y147R, Q154R, |
| ABE9.26_heterodimer | E25F, V82S, Y123H, D139M, Y147R, Q154R |
| ABE9.27 _heterodimer | M70V, V82S, M94V, Y123H, Y147R, Q154R |
| ABE9.28_heterodimer | Q71M, V82S, Y123H, Y147R, Q154R |
| ABE9.29_monomer | E25F_I76Y_V82S_Y123H_Y147R_Q154R |
| ABE9.30_monomer | I76Y_V82T_Y123H_Y147R_Q154R |
| ABE9.31_monomer | N38G_I76Y_V82S_Y123H_Y147R_Q154R |
| ABE9.32_monomer | N38G_I76Y_V82T_Y123H_Y147R_Q154R |
| ABE9.33_monomer | R23H_I76Y_V82S_Y123H_Y147R_Q154R |
| ABE9.34_monomer | P54C_I76Y_V82S_Y123H_Y147R_Q154R |
| ABE9.35_monomer | R21N_I76Y_V82S_Y123H_Y147R_Q154R |
| ABE9.36_monomer | I76Y_V82S_Y123H_D138M_Y147R_Q154R |
| ABE9.37_monomer | Y72S_I76Y_V82S_Y123H_Y147R_Q154R |
| ABE9.38_heterodimer | E25F_I76Y_V82S_Y123H_Y147R_Q154R |
| ABE9.39_heterodimer | I76Y_V82T_Y123H_Y147R_Q154R |
| ABE9.40_heterodimer | N38G_I76Y_V82S_Y123H_Y147R_Q154R |
| ABE9.41_heterodimer | N38G_I76Y_V82T_Y123H_Y147R_Q154R |
| ABE9.42_heterodimer | R23H_I76Y_V82S_Y123H_Y147R_Q154R |
| ABE9.43_heterodimer | P54C_I76Y_V82S_Y123H_Y147R_Q154R |
| ABE9.44_heterodimer | R21N_I76Y_V82S_Y123H_Y147R_Q154R |
| ABE9.45_heterodimer | I76Y_V82S_Y123H_D138M_Y147R_Q154R |
| ABE9.46_heterodimer | Y72S_I76Y_V82S_Y123H_Y147R_Q154R |
| ABE9.47_monomer | N72K_V82S, Y123H, Y147R, Q154R |
| ABE9.48_monomer | Q71M_V82S, Y123H, Y147R, Q154R |
| ABE9.49_monomer | M70V, V82S, M94V, Y123H, Y147R, Q154R |
| ABE9.50_monomer | V82S, Y123H, T133K, Y147R, Q154R |
| ABE9.51_monomer | V82S, Y123H, T133K, Y147R, Q154R, A158K |
| ABE9.52_monomer | M70V, Q71M, N72K, V82S, Y123H, Y147R, Q154R |
| ABE9.53_heterodimer | N72K_V82S, Y123H, Y147R, Q154R |
| ABE9.54_heterodimer | Q71M_V82S, Y123H, Y147R, Q154R |
| ABE9.55_heterodimer | M70V, V82S, M94V, Y123H, Y147R, Q154R |
| ABE9.56_heterodimer | V82S, Y123H, T133K, Y147R, Q154R |
| ABE9.57_heterodimer | V82S, Y123H, T133K, Y147R, Q154R, A158K |
| ABE9.58_heterodimer | M70V, Q71M, N72K, V82S, Y123H, Y147R, Q154R |

In some embodiments, the base editor comprises a domain comprising all or a portion of a uracil glycosylase inhibitor (UGI). In some embodiments, the base editor comprises a domain comprising all or a portion of a nucleic acid polymerase. In some embodiments, a base editor can comprise as a domain all or a portion of a nucleic acid polymerase (NAP). For example, a base editor can comprise all or a portion of a eukaryotic NAP. In some embodiments, a NAP or portion thereof incorporated into a base editor is a DNA polymerase. In some embodiments, a NAP or portion thereof incorporated into a base editor has translesion polymerase activity. In some embodiments, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase). In some embodiments, a nucleic acid polymerase or portion thereof incorporated into a base editor is a translesion DNA polymerase.

In some embodiments, a domain of the base editor can comprise multiple domains. For example, the base editor comprising a polynucleotide programmable nucleotide binding domain derived from Cas9 can comprise a REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild-type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, etc.).

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated.

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)n (SEQ ID NO: 1308), (GGGGS)n (SEQ ID NO: 109), and (G)n to more rigid linkers of the form (EAAAK)n (SEQ ID NO: 1309), (SGGS)n (SEQ ID NO: 57), SGSETPGTSESATPES (SEQ ID NO: 56) (see, e.g., Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7 (SEQ ID NO: 4039). In some embodiments, cytidine deaminase or adenosine deaminase and the Cas9 domain of any of the fusion proteins provided herein are fused via a linker comprising the amino acid sequence SGSETPGTSESATPES (SEQ ID NO: 56), which can also be referred to as the XTEN linker. In some embodiments, a linker comprises a plurality of proline residues and is 5-21, 5-14, 5-9, 5-7 amino acids in length, e.g., PAPAP (SEQ ID NO: 65), PAPAPA (SEQ ID NO: 66), PAPAPAP (SEQ ID NO: 67), PAPAPAPA (SEQ ID NO: 68), P(AP)4 (SEQ ID NO: 69), P(AP)7 (SEQ ID NO: 70), P(AP)10 (SEQ ID NO: 71) (see, e.g., Tan J, Zhang F, Karcher D, Bock R. Engineering of high-precision base editors for site-specific single nucleotide replacement. Nat Commun. 2019 January 25; 10(1):439; the entire contents are incorporated herein by reference). Such proline-rich linkers are also termed “rigid” linkers.

In another embodiment, the base editor system comprises a component (protein) that interacts non-covalently with a deaminase (DNA deaminase), e.g., an adenosine or a cytidine deaminase, and transiently attracts the adenosine or cytidine deaminase to the target nucleobase in a target polynucleotide sequence for specific editing, with minimal or reduced bystander or target-adjacent effects. Such a non-covalent system and method involving deaminase-interacting proteins serves to attract a DNA deaminase to a particular genomic target nucleobase and decouples the events of on-target and target-adjacent editing, thus enhancing the achievement of more precise single base substitution mutations. In an embodiment, the deaminase-interacting protein binds to the deaminase (e.g., adenosine deaminase or cytidine deaminase) without blocking or interfering with the active (catalytic) site of the deaminase from engaging the target nucleobase (e.g., adenosine or cytidine, respectively). Such as system, termed “MagnEdit,” involves interacting proteins tethered to a Cas9 and gRNA complex and can attract a co-expressed adenosine or cytidine deaminase (either exogenous or endogenous) to edit a specific genomic target site, and is described in McCann, J. et al., 2020, “MagnEdit—interacting factors that recruit DNA-editing enzymes to single base targets,” Life-Science-Alliance, Vol. 3, No. 4 (e201900606), (doi 10.26508/Isa.201900606), the contents of which are incorporated by reference herein in their entirety. In an embodiment, the DNA deaminase is an adenosine deaminase variant (e.g., TadA*8) as described herein.

In another embodiment, a system called “Suntag,” involves non-covalently interacting components used for recruiting protein (e.g., adenosine deaminase or cytidine deaminase) components, or multiple copies thereof, of base editors to polynucleotide target sites to achieve base editing at the site with reduced adjacent target editing, for example, as described in Tanenbaum, M. E. et al., “A protein tagging system for signal amplification in gene expression and fluorescence imaging,” Cell. 2014 Oct. 23; 159(3): 635-646. doi:10.1016/j.cell.2014.09.039; and in Huang, Y.-H. et al., 2017, “DNA epigenome editing using CRISPR-Cas SunTag-directed DNMT3A,” Genome Biol 18: 176. doi:10.1186/s13059-017-1306-z, the contents of each of which are incorporated by reference herein in their entirety. In an embodiment, the DNA deaminase is an adenosine deaminase variant (e.g., TadA*8) as described herein.

Nucleic Acid Programmable DNA Binding Proteins with Guide RNAs

Provided herein are compositions and methods for base editing in cells. Further provided herein are compositions comprising a guide polynucleic acid sequence, e.g. a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g. a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide complexes comprising any of the fusion proteins provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 6 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence. In some embodiments, the 3′ end of the target sequence is immediately adjacent to an e.g., TTN, DTTN, GTTN, ATTN, ATTC, DTTNT, WTTN, HATY, TTTN, TTTV, TTTC, TG, RTR, or YTN PAM site.

It will be understood that the numbering of the specific positions or residues in the respective sequences depends on the particular protein and numbering scheme used. Numbering might differ, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins disclosed herein, to a target site, e.g., a site comprising a mutation to be edited, it is typically necessary to co-express the fusion protein together with a guide RNA. As explained in more detail elsewhere herein, a guide RNA typically comprises a tracrRNA framework allowing for napDNAbp (e.g., Cas9 or Cas12) binding, and a guide sequence, which confers sequence specificity to the napDNAbp:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting napDNAbp:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

Distinct portions of sgRNA are predicted to form various features that interact with Cas9 (e.g., SpyCas9) and/or the DNA target. Six conserved modules have been identified within native crRNA:tracrRNA duplexes and single guide RNAs (sgRNAs) that direct Cas9 endonuclease activity (see Briner et al., Guide RNA Functional Modules Direct Cas9 Activity and Orthogonality Mol Cell. 2014 Oct. 23; 56(2):333-339). The six modules include the spacer responsible for DNA targeting, the upper stem, bulge, lower stem formed by the CRISPR repeat:tracrRNA duplex, the nexus, and hairpins from the 3′ end of the tracrRNA. The upper and lower stems interact with Cas9 mainly through sequence-independent interactions with the phosphate backbone. In some embodiments, the upper stem is dispensable. In some embodiments, the conserved uracil nucleotide sequence at the base of the lower stem is dispensable. The bulge participates in specific side-chain interactions with the Rec1 domain of Cas9. The nucleobase of U44 interacts with the side chains of Tyr 325 and His 328, while G43 interacts with Tyr 329. The nexus forms the core of the sgRNA:Cas9 interactions and lies at the intersection between the sgRNA and both Cas9 and the target DNA. The nucleobases of A51 and A52 interact with the side chain of Phe 1105; U56 interacts with Arg 457 and Asn 459; the nucleobase of U59 inserts into a hydrophobic pocket defined by side chains of Arg 74, Asn 77, Pro 475, Leu 455, Phe 446, and Ile 448; C60 interacts with Leu 455, Ala 456, and Asn 459, and C61 interacts with the side chain of Arg 70, which in turn interacts with C15. In some embodiments, one or more of these mutations are made in the bulge and/or the nexus of a sgRNA for a Cas9 (e.g., spyCas9) to optimize sgRNA:Cas9 interactions.

Moreover, the tracrRNA nexus and hairpins are critical for Cas9 pairing and can be swapped to cross orthogonality barriers separating disparate Cas9 proteins, which is instrumental for further harnessing of orthogonal Cas9 proteins. In some embodiments, the nexus and hairpins are swapped to target orthogonal Cas9 proteins. In some embodiments, a sgRNA is dispensed of the upper stem, hairpin 1, and/or the sequence flexibility of the lower stem to design a guide RNA that is more compact and conformationally stable. In some embodiments, the modules are modified to optimize multiplex editing using a single Cas9 with various chimeric guides or by concurrently using orthogonal systems with different combinations of chimeric sgRNAs. Details regarding guide functional modules and methods thereof are described, for example, in Briner et al., Guide RNA Functional Modules Direct Cas9 Activity and Orthogonality Mol Cell. 2014 Oct. 23; 56(2):333-339, the contents of which is incorporated by reference herein in its entirety.

The domains of the base editor disclosed herein can be arranged in any order. Non-limiting examples of a base editor comprising a fusion protein comprising e.g., a polynucleotide-programmable nucleotide-binding domain (e.g., Cas9 or Cas12) and a deaminase domain (e.g., cytidine or adenosine deaminase) can be arranged as follows:

NH2-[nucleobase editing domain]-Linker1-[nucleobase editing domain]-COOH;

NH2-[deaminase]-Linker1-[nucleobase editing domain]-COOH;

NH2-[deaminase]-Linker1-[nucleobase editing domain]-Linker2-[UGI]-COOH;

NH2-[deaminase]-Linker1-[nucleobase editing domain]-COOH;

NH2-[adenosine deaminase]-Linker1-[nucleobase editing domain]-COOH;

NH2-[nucleobase editing domain]-[deaminase]-COOH;

NH2-[deaminase]-[nucleobase editing domain]-[inosine BER inhibitor]-COOH;

NH2-[deaminase]-[inosine BER inhibitor]-[nucleobase editing domain]-COOH;

NH2-[inosine BER inhibitor]-[deaminase]-[nucleobase editing domain]-COOH;

NH2-[nucleobase editing domain]-[deaminase]-[inosine BER inhibitor]-COOH;

NH2-[nucleobase editing domain]-[inosine BER inhibitor]-[deaminase]-COOH;

NH2-[inosine BER inhibitor]-[nucleobase editing domain]-[deaminase]-COOH;

NH2-[nucleobase editing domain]-Linker1-[deaminase]-Linker2-[nucleobase editing domain]-COOH;

NH2-[nucleobase editing domain]-Linker1-[deaminase]-[nucleobase editing domain]-COOH;

NH2-[nucleobase editing domain]-[deaminase]-Linker2-[nucleobase editing domain]-COOH;

NH2-[nucleobase editing domain]-[deaminase]-[nucleobase editing domain]-COOH;

NH2-[nucleobase editing domain]-Linker1-[deaminase]-Linker2-[nucleobase editing domain]-[inosine BER inhibitor]-COOH;

NH2-[nucleobase editing domain]-Linker1-[deaminase]-[nucleobase editing domain]-[inosine BER inhibitor]-COOH;

NH2-[nucleobase editing domain]-[deaminase]-Linker2-[nucleobase editing domain]-[inosine BER inhibitor]-COOH;

NH2-[nucleobase editing domain]-[deaminase]-[nucleobase editing domain]-[inosine BER inhibitor]-COOH;

NH2-[inosine BER inhibitor]-[nucleobase editing domain]-Linker1-[deaminase]-Linker2-[nucleobase editing domain]-COOH;

NH2-[inosine BER inhibitor]-[nucleobase editing domain]-Linker1-[deaminase]-[nucleobase editing domain]-COOH;

NH2-[inosine BER inhibitor]-[nucleobase editing domain]-[deaminase]-Linker2-[nucleobase editing domain]-COOH; or

NH2-[inosine BER inhibitor]NH2-[nucleobase editing domain]-[deaminase]-[nucleobase editing domain]-COOH.

In some embodiments, the base editing fusion proteins provided herein need to be positioned at a precise location, for example, where a target base is placed within a defined region (e.g., a “deamination window”). In some embodiments, a target can be within a 4-base region. In some embodiments, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a napDNAbp domain. In some embodiments, an NLS of the base editor is localized C-terminal to a napDNAbp domain.

Non-limiting examples of protein domains which can be included in the fusion protein include a deaminase domain (e.g., adenosine deaminase or cytidine deaminase), a uracil glycosylase inhibitor (UGI) domain, epitope tags, reporter gene sequences, and/or protein domains having one or more of the activities described herein.

A domain may be detected or labeled with an epitope tag, a reporter protein, other binding domains. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions.

### Methods of Using Fusion Proteins Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA described herein.

In some embodiments, a fusion protein of the invention is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins comprising a Cas9 domain and a cytidine or adenosine deaminase, as disclosed herein, to a target site, e.g., a site comprising a mutation to be edited, a guide RNA, e.g., an sgRNA, may be co-expressed. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations. In some embodiments, an intended mutation is a mutation that is generated by a specific base editor (e.g., adenosine base editor or cytidine base editor) bound to a guide polynucleotide (e.g., gRNA), specifically designed to generate the intended mutation. In some embodiments, the intended mutation is in a gene associated with a target antigen associated with a disease or disorder, e.g., a neurological or ophthalmological disease or disorder. In some embodiments, the intended mutation is an adenine (A) to guanine (G) point mutation (e.g., SNP) in a gene associated with a target antigen associated with a disease or disorder, e.g a neurological or ophthalmological disease or disorder. In some embodiments, the intended mutation is an adenine (A) to guanine (G) point mutation within the coding region or non-coding region of a gene (e.g., regulatory region or element). In some embodiments, the intended mutation is a cytosine (C) to thymine (T) point mutation (e.g., SNP) in a gene associated with a target antigen associated with a disease or disorder, e.g., a neurological or ophthalmological disease or disorder. In some embodiments, the intended mutation is a cytosine (C) to thymine (T) point mutation within the coding region or non-coding region of a gene (e.g., regulatory region or element). In some embodiments, the intended mutation is a point mutation that generates a STOP codon, for example, a premature STOP codon within the coding region of a gene. In some embodiments, the intended mutation is a mutation that eliminates a stop codon.

The base editors of the invention advantageously modify a specific nucleotide base encoding a protein without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate) a specific nucleotide within a nucleic acid, without generating a large number of insertions or deletions (i.e., indels) in the nucleic acid. In some embodiments, it is desirable to generate base editors that efficiently modify (e.g. mutate or methylate) a specific nucleotide within a nucleic acid, without generating a large number of insertions or deletions (i.e., indels) in the nucleic acid. In certain embodiments, any of the base editors provided herein can generate a greater proportion of intended modifications (e.g., methylations) versus indels. In certain embodiments, any of the base editors provided herein can generate a greater proportion of intended modifications (e.g., mutations) versus indels.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation in a nucleic acid (e.g. a nucleic acid within a genome of a subject) without generating a considerable number of unintended mutations (e.g., spurious off-target editing or bystander editing). In some embodiments, an intended mutation is a mutation that is generated by a specific base editor bound to a gRNA, specifically designed to generate the intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended mutations:unintended mutations) that is greater than 1:1. In some embodiments, any of the base editors provided herein are capable of generating a ratio of intended mutations to unintended mutations that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described herein may be applied to any of the fusion proteins, or methods of using the fusion proteins provided herein.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence, and may affect the gene product. In essence therefore, the gene editing modification described herein may result in a modification of the gene, structurally and/or functionally, wherein the expression of the gene product may be modified, for example, the expression of the gene is knocked out; or conversely, enhanced, or, in some circumstances, the gene function or activity may be modified. Using the methods disclosed herein, a base editing efficiency may be determined as the knockdown efficiency of the gene in which the base editing is performed, wherein the base editing is intended to knockdown the expression of the gene. A knockdown level may be validated quantitatively by determining the expression level by any detection assay, such as assay for protein expression level, for example, by flow cytometry; assay for detecting RNA expression such as quantitative RT-PCR, northern blot analysis, or any other suitable assay such as pyrosequencing; and may be validated qualitatively by nucleotide sequencing reactions.

In some embodiments, the modification, e.g., single base edit results in at least 10% reduction of the gene targeted expression. In some embodiments, the base editing efficiency may result in at least 10% reduction of the gene targeted expression. In some embodiments, the base editing efficiency may result in at least 20% reduction of the gene targeted expression. In some embodiments, the base editing efficiency may result in at least 30% reduction of the gene targeted expression. In some embodiments, the base editing efficiency may result in at least 40% reduction of the gene targeted expression. In some embodiments, the base editing efficiency may result in at least 50% reduction of the gene targeted expression. In some embodiments, the base editing efficiency may result in at least 60% reduction of the targeted gene expression. In some embodiments, the base editing efficiency may result in at least 70% reduction of the targeted gene expression. In some embodiments, the base editing efficiency may result in at least 80% reduction of the targeted gene expression. In some embodiments, the base editing efficiency may result in at least 90% reduction of the targeted gene expression. In some embodiments, the base editing efficiency may result in at least 91% reduction of the targeted gene expression. In some embodiments, the base editing efficiency may result in at least 92% reduction of the targeted gene expression. In some embodiments, the base editing efficiency may result in at least 93% reduction of the targeted gene expression. In some embodiments, the base editing efficiency may result in at least 94% reduction of the targeted gene expression. In some embodiments, the base editing efficiency may result in at least 95% reduction of the targeted gene expression. In some embodiments, the base editing efficiency may result in at least 96% reduction of the targeted gene expression. In some embodiments, the base editing efficiency may result in at least 97% reduction of the targeted gene expression. In some embodiments, the base editing efficiency may result in at least 98% reduction of the targeted gene expression. In some embodiments, the base editing efficiency may result in at least 99% reduction of the targeted gene expression. In some embodiments, the base editing efficiency may result in knockout (100% knockdown of the gene expression) of the gene that is targeted.

In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, targeted modifications, e.g., single base editing, are used simultaneously to target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 different endogenous sequences for base editing with different guide RNAs. In some embodiments, targeted modifications, e.g. single base editing, are used to sequentially target at least 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50, or more different endogenous gene sequences for base editing with different guide RNAs.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e., at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, any of base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence. In some embodiments, any of base editor systems comprising one of the ABE8 base editor variants described herein result in less than 0.8% indel formation in the target polynucleotide sequence. In some embodiments, any of base editor systems comprising one of the ABE8 base editor variants described herein result in at most 0.8% indel formation in the target polynucleotide sequence. In some embodiments, any of base editor systems comprising one of the ABE8 base editor variants described herein result in less than 0.3% indel formation in the target polynucleotide sequence. In some embodiments, any of base editor systems comprising one of the ABE8 base editor variants described results in lower indel formation in the target polynucleotide sequence compared to a base editor system comprising one of ABE7 base editors. In some embodiments, any of base editor systems comprising one of the ABE8 base editor variants described herein results in lower indel formation in the target polynucleotide sequence compared to a base editor system comprising an ABE7.10.

In some embodiments, any of base editor systems comprising one of the ABE8 base editor variants described herein has reduction in indel frequency compared to a base editor system comprising one of the ABE7 base editors. In some embodiments, any of base editor systems comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The invention provides adenosine deaminase variants (e.g., ABE8 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE8 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 base editor variants described herein has reduced bystander editing or mutations compared to a base editor system comprising an ABE7 base editor, e.g., ABE7.10. In some embodiments, any of the base editing system comprising one of the ABE8 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE8 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 base editor variants described herein has reduced spurious editing compared to a base editor system comprising an ABE7 base editor, e.g., ABE7.10. In some embodiments, any of the base editing system comprising one of the ABE8 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA. In some embodiments, an ABE8 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 base editor delivered by an mRNA system has higher base editing efficiency compared to an ABE8 base editor delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 base editor variants described herein has lower guided off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 base editor variants described herein has at least about 2.2 fold decrease in guided off-target editing efficiency when delivered by an mRNA system compared to when delivered by a plasmid or vector system.

In some embodiments, any of the ABE8 base editor variants described herein has lower guide-independent off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 base editor variants described herein does not increase guide-independent mutation rates across the genome.

In some embodiments, a single gene delivery event (e.g., by transduction, transfection, electroporation or any other method) can be used to target base editing of 5 sequences within a cell's genome. In some embodiments, a single gene delivery event can be used to target base editing of 6 sequences within a cell's genome. In some embodiments, a single gene delivery event can be used to target base editing of 7 sequences within a cell's genome. In some embodiments, a single electroporation event can be used to target base editing of 8 sequences within a cell's genome. In some embodiments, a single gene delivery event can be used to target base editing of 9 sequences within a cell's genome. In some embodiments, a single gene delivery event can be used to target base editing of 10 sequences within a cell's genome. In some embodiments, a single gene delivery event can be used to target base editing of 20 sequences within a cell's genome. In some embodiments, a single gene delivery event can be used to target base editing of 30 sequences within a cell's genome. In some embodiments, a single gene delivery event can be used to target base editing of 40 sequences within a cell's genome. In some embodiments, a single gene delivery event can be used to target base editing of 50 sequences within a cell's genome.

In some embodiments, the method described herein, for example, the base editing methods has minimum to no off-target effects.

In some embodiments, the base editing method described herein results in at least 50% of a cell population that have been successfully edited (i.e., cells that have been successfully engineered). In some embodiments, the base editing method described herein results in at least 55% of a cell population that have been successfully edited. In some embodiments, the base editing method described herein results in at least 60% of a cell population that have been successfully edited. In some embodiments, the base editing method described herein results in at least 65% of a cell population that have been successfully edited. In some embodiments, the base editing method described herein results in at least 70% of a cell population that have been successfully edited. In some embodiments, the base editing method described herein results in at least 75% of a cell population that have been successfully edited. In some embodiments, the base editing method described herein results in at least 80% of a cell population that have been successfully edited. In some embodiments, the base editing method described herein results in at least 85% of a cell population that have been successfully edited. In some embodiments, the base editing method described herein results in at least 90% of a cell population that have been successfully edited. In some embodiments, the base editing method described herein results in at least 95% of a cell population that have been successfully edited. In some embodiments, the base editing method described herein results in about 91%, 92%, 93%, 94%, 95%, 96%, 97%, 98%, 99% or 100% of a cell population that have been successfully edited.

In some embodiments, the live cell recovery following a base editing intervention is greater than at least 60%, 70%, 80%, 90% of the starting cell population at the time of the base editing event. In some embodiments, the live cell recovery as described above is about 70%. In some embodiments, the live cell recovery as described above is about 75%. In some embodiments, the live cell recovery as described above is about 80%. In some embodiments, the live cell recovery as described above is about 85%. In some embodiments, the live cell recovery as described above is about 90%, or about 91%, 92%, 93%, 94% 95%, 96%, 97%, 98%, or 99%, or 100% of the cells in the population at the time of the base editing event.

In some embodiments the engineered cell population can be further expanded in vitro by about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

Details of base editor efficiency are described in International PCT Application Nos. PCT/2017/045381 (WO 2018/027078) and PCT/US2016/058344 (WO 2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference. In some embodiments, editing of a plurality of nucleobase pairs in one or more genes using the methods provided herein results in formation of at least one intended mutation. In some embodiments, said formation of said at least one intended mutation results in the disruption the normal function of a gene. In some embodiments, said formation of said at least one intended mutation results decreases or eliminates the expression of a protein encoded by a gene. It should be appreciated that multiplex editing can be accomplished using any method or combination of methods provided herein.

### Engineered Nucleases

In some embodiments, the gene editing system comprises an engineered nuclease (e.g., meganuclease, zinc finger nuclease (ZFN), Transcription activator-like effector nuclease (TALEN), or a Cas nuclease. In some embodiments, the gene editing system comprises a ZFN. ZFNs are fusion proteins comprising a zinc-finger DNA binding domain (“ZF”) and a nuclease domain. Each naturally-occurring ZF may bind to three consecutive base pairs (a DNA triplet), and ZF repeats are combined to recognize a DNA target sequence and provide sufficient affinity. Thus, engineered ZF repeats are combined to recognize longer DNA sequences, such as, e.g., 9 base pairs, 12 base pairs, 15 base pairs, 18 base pairs, etc. In some embodiments, the ZFN comprise ZFs fused to a nuclease domain from a restriction endonuclease (e.g., FokI). In some embodiments, the nuclease domain comprises a dimerization domain, such as when the nuclease dimerizes to be active, and a pair of ZFNs comprising the ZF repeats and the nuclease domain is designed for targeting a target sequence, which comprises two half target sequences recognized by each ZF repeats on opposite strands of the DNA molecule, with an interconnecting sequence in between (which is sometimes called a spacer in the literature). For example, the interconnecting sequence is 5 to 7 basepairs in length. When both ZFNs of the pair bind, the nuclease domain may dimerize and introduce a DSB within the interconnecting sequence. In some embodiments, the dimerization domain of the nuclease domain comprises a knob-into-hole motif to promote dimerization.

In some embodiments, the gene editing system comprises a TALEN. The DNA binding domain of TALENs usually comprises a variable number of 34 or 35 amino acid repeats (“modules” or “TAL modules”), with each module binding to a single DNA base pair, A, T, G, or C. Adjacent residues at positions 12 and 13 (the “repeat-variable di-residue” or RVD) of each module specify the single DNA base pair that the module binds to. In some embodiments, the TALEN may comprise a nuclease domain from a restriction endonuclease (e.g., FokI). In some embodiments, the nuclease domain may dimerize to be active, and a pair of TALENS is designed for targeting a target sequence, which comprises two half target sequences recognized by each DNA binding domain on opposite strands of the DNA molecule, with an interconnecting sequence in between. For example, each half target sequence is in the range of 10 to 20 base pairs, and the interconnecting sequence is 12 to 19 base pairs in length. When both TALENs of the pair bind, the nuclease domain may dimerize and introduce a double strand break within the interconnecting sequence. In some embodiments, the dimerization domain of the nuclease domain may comprise a knob-into-hole motif to promote dimerization.

In some embodiments, the gene editing system comprises a meganuclease. Naturally-occurring meganucleases recognize and cleave double-stranded DNA sequences of about 12 to 40 base pairs and are commonly grouped into five families. In some embodiments, the meganuclease is chosen from the LAGLIDADG family, the GIY-YIG family, the HNH family, the His-Cys box family, and the PD-(D/E)XK family. In some embodiments, the DNA binding domain of the meganuclease is engineered to recognize and bind to a sequence other than its cognate target sequence. In some embodiments, the DNA binding domain of the meganuclease is fused to a heterologous nuclease domain. In some embodiments, the meganuclease, such as a homing endonuclease, are fused to TAL modules to create a hybrid protein, such as a “megaTAL” protein. The megaTAL proteins can have improved DNA targeting specificity by recognizing the target sequences of both the DNA binding domain of the meganuclease and the TAL modules.

### G. PHARMACEUTICAL COMPOSITIONS AND FORMULATIONS

Provided herein are compositions (e.g., pharmaceutical compositions) comprising any of the recombinant rabies virus genomes and recombinant rabies viruses described herein. The term “pharmaceutical composition,” as used herein, refers to a composition formulated for pharmaceutical use. In certain embodiments, the pharmaceutical composition further comprises a pharmaceutically acceptable carrier. In certain embodiments, the pharmaceutical composition comprises additional agents (e.g., for specific delivery, increasing half-life, or other therapeutic compounds).

As used herein, the term “pharmaceutically-acceptable carrier” refers to a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound (e.g., a recombinant rabies virus genome or recombinant rabies virus described herein) from one site (e.g., the delivery site) of the body, to another site (e.g., a target organ, tissue, or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid, such as histidine, or a mixture of amino acids, such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g., tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In certain embodiments, the pharmaceutical composition is formulated for delivery to a subject, e.g., for gene therapy. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In certain embodiments, the pharmaceutical composition described herein is administered locally to a diseased site (e.g., tumor site). In certain embodiments, the pharmaceutical composition described herein is administered to a subject by injection, by means of a catheter, by means of a suppository, or by means of an implant, the implant being of a porous, non-porous, or gelatinous material, including a membrane, such as a silastic membrane, or a fiber.

In certain embodiments, the pharmaceutical composition described herein is delivered in a controlled release system. In certain embodiments, a pump can be used (see, e.g., Langer, 1990, Science 249: 1527-1533; Sefton, 1989, CRC Crit. Ref. Biomed. Eng. 14:201; Buchwald et al, 1980, Surgery 88:507; Saudek et al., 1989, N. Engl. J. Med. 321:574). In certain embodiments, polymeric materials can be used. See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See, also, Levy et al, 1985, Science 228: 190; During et al, 1989, Ann. Neurol. 25:351; Howard et ah, 1989, J. Neurosurg. 71: 105. Other controlled release systems are discussed, for example, in Langer, supra.

In certain embodiments, the pharmaceutical composition is formulated in accordance with routine procedures as a composition adapted for intravenous or subcutaneous administration to a subject, e.g., a human. In certain embodiments, pharmaceutical compositions for administration by injection are solutions in sterile isotonic used as solubilizing agent and a local anesthetic such as lignocaine to ease pain at the site of the injection.

Generally, the ingredients are supplied either separately or mixed together in unit dosage form, for example, as a dry lyophilized powder or water free concentrate in a hermetically sealed container such as an ampoule or sachette indicating the quantity of active agent.

Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (see, e.g., Zhang Y. P. et al., Gene Ther. 1999, 6: 1438-47). Positively charged lipids such as 1,2-dioleoyl-3-trimethylammonium-propane, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile, used for reconstitution or dilution of the lyophilized compound of the invention). Optionally associated with such containers) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In certain embodiments, the article of manufacture comprises a container and a label. Suitable containers include, for example, bottles, vials, syringes, and test tubes. The containers can be formed from a variety of materials such as glass or plastic. In certain embodiments, the container holds a composition (e.g., a recombinant rabies virus genome or a recombinant rabies virus described herein) that is effective for treating a disease and can have a sterile access port. For example, the container can be an intravenous solution bag or a vial having a stopper pierceable by a hypodermic injection needle. The active agent in the composition is a compound (e.g., a recombinant rabies virus genome or a recombinant rabies virus) of the disclosure. In certain embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the recombinant rabies virus genomes or recombinant rabies viruses described herein are provided as part of a pharmaceutical composition. In some embodiments, the pharmaceutical composition comprises any of the recombinant rabies virus genomes or recombinant rabies viruses described herein. In some embodiments, the pharmaceutical composition comprises any of the complexes provided herein.

In some embodiments, compositions provided herein are administered to a subject, for example, to a human subject, in order to effect a targeted genomic modification within the subject. In some embodiments, cells are obtained from the subject and contacted with any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated in its entirety herein by reference) discloses various excipients used in formulating pharmaceutical compositions and known techniques for the preparation thereof. See also PCT application PCT/US2010/055131 (Publication number WO2011053982 A8, filed Nov. 2, 2010), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure. In certain embodiments, compositions in accordance with the present invention may be used for treatment of any of a variety of diseases, disorders, and/or conditions.

Various aspects of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology,” and “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the various aspects of the present disclosure, and, as such, may be considered in making and practicing the same.

### H. POLYNUCLEOTIDES, VECTORS, AND CELLS

Provided herein are polynucleotides comprising: (i) a recombinant rabies virus genome described herein; (ii) an N gene encoding for a rabies virus nucleoprotein or a functional variant thereof; (iii) a P gene encoding for a rabies virus phosphoprotein or a functional variant thereof; (iv) an L gene encoding for a rabies virus polymerase (e.g., a RNA-dependent RNA polymerase) or a functional variant thereof; (v) a G gene encoding for a rabies virus glycoprotein or a functional variant thereof; and/or (vi) an M gene encoding for a rabies virus matrix protein or a functional variant thereof.

The polynucleotides described herein can be obtained by any method known in the art, such as by chemically synthesizing the DNA chain, by PCR, or by the Gibson Assembly method. The advantage of constructing a full-length DNA by chemical synthesis or a combination of PCR method or Gibson Assembly method is that the codons may be optimized to ensure that the fusion protein is expressed at a high level in a host cell. Optimized codons may be selected using the genetic code use frequency database (http://www.kazusa.or.jp/codon/index.html), which is disclosed in the home page of Kazusa DNA Research Institute. In certain embodiments, the polynucleotide is codon optimized. In certain embodimens, the polynucleotide can be obtimized by RNA optimization. Additional optimization methods can be included to increase stability for recombinant expression, including, e.g., replacement of signal sequences with exogenous signal sequences, removal of instability elements, removal of inhibitory regions, removal of potential splice sites, and other optimization methods known to those of ordinary skill in the art. See, e.g., U.S. Pat. No. 6,794,498, the disclosure of which is herein incorporated by reference in its entirety.

Once obtained, polynucleotides of the present disclosure may be incorporated into suitable expression vectors. Accordingly, the present disclosure also provides a vector comprising any of the polynucleotides disclosed herein, separately, or in combination. Suitable vectors include plasmids, viruses, artificial chromosomes, bacmids, cosmids, and others known to those of ordinary skill in the art. In certain embodiments, the vector is an expression vector.

Suitable expression vectors include Escherichia coli-derived plasmids (e.g., pBR322, pBR325, pUC12, pUC13); Bacillus subtilis-derived plasmids (e.g., pUB110, pTP5, pCI94); yeast-derived plasmids (e.g., pSH19, pSH15); plasmids suitable for expression in insect cells (e.g., pFast-Bac); plasmids suitable for expression in mammalian cells (e.g., pXTI, pRc/CMV, pRc/RSV, pcDNA1/Neo); also bacteriophages, such as lamda phage and the like; other vectors that may be used include insect viral vectors, such as baculovirus and the like (e.g., BmNPV, AcNPV); and viral vectors suitable for expression in a mammalian cell, such as retrovirus, vaccinia virus, adenovirus and the like.

The genes and/or transgenes comprises with the polynucleotides and vectors are typically expressed under the control of a transcriptional regulatory element. In certain embodiments, the transcriptional regulatory element can comprise one or more enhancer elements, intron elements, and/or promoter elements. In certain embodiments, the transcriptional regulatory element comprises a constitutive promoter. Examples of transcriptional regulatory elements include those that comprise a CMV promoter (promoter from human cytomegalovirus) optionally including a CMV enhancer, a EF1α promoter (promoter from human elongation factor 1 alpha), a CBA promoter (comprising a CMV early enhancer and a chicken β-actin promoter), a CAG promoter (comprising a CBA promoter and a rabbit β-globin intron), a CAGGS promoter (comprising a CMV enhancer, a CBA promoter, and chicken β-actin intron 1/exon 1), a PGK promoter (promoter from phosphoglycerate kinase), a U6 promoter (U6 nuclear promoter), a Ubc promoter (promoter from human ubiquitin C), a CASI promoter (comprising a CMV enhancer, a ubiquitin C enhancer, and a chicken β-actin promoter), and a CALM1 promoter (promoter from calmodulin 1). Various constitutive transcriptional regulatory elements are known to those of ordinary skill in the art.

In certain embodiments, the transcriptional regulatory element comprises an inducible promoter. For example, the transcripitional regulatory element can comprise the inducible TRE promoter (tetracyclin response element promoter). Such inducible promoters can be positive inducible, where the promoter is inactive because an activator protein cannot bind thereto, or negative inducible, wherein a repressor protein is bound thereto that prevents transcription. Examples of inducible promoters include those that are chemically inducible, e.g., a tetracycline ON (Tet-On) promoter system, a lac repressor system, a pBad prokaryotic promoter, and others such as alcohol or steroid regulated promoters. Inducible promoters can be temperature inducible, e.g., heat or cold induced promoters (e.g., Hsp70 or Hsp90-derived promoters), and light inducible, where light can be used to regulate transcription. In certain embodiments, the transcriptional regulatory element comprises a repressible promoter. Various inducible transcriptional regulatory elements are known to those of ordinary skill in the art.

In certain embodiments, the transcriptional regulatory element comprises an promoter exogenous to the gene or transgene. In certain embodiments, the transcriptional regulatory element comprises a synthetic promoter.

Suitable promoters may be chosen based on its use for expression in a desired host cell. For example, when the host is an animal cell, any one of the following promoters are used: SR-alpha promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. In certain embodiments, the promoter is CMV promoter or SR alpha promoter. In certain embodiments, the promoter is an elongation factor 1-alpha (EF1a) promoter. When the host cell is Escherichia coli, any of the following promoters may be used: trp promoter, lac promoter, recA promoter, lambdaPL promoter, Ipp promoter, T7 promoter and the like. When the host is genus Bacillus, any of the following promoters may be used: SPO1 promoter, SPO2 promoter, penP promoter and the like. When the host is a yeast, any of the following promoters may be used: Gal1/10 promoter, PHOS promoter, PGK promoter, GAP promoter, ADH promoter and the like. When the host is an insect cell, any of the following promoters may be used: polyhedrin promoter, P10 promoter and the like. When the host is a plant cell, any of the following promoters may be used: CaMV35S promoter, CaMVI9S promoter, NOS promoter and the like.

If desired, the expression vector also includes any one or more of an enhancer, splicing signal, terminator, polyadenylation signal, a selection marker (e.g., a drug resistance gene, auxotrophic complementary gene and the like), or a replication origin.

The polynucleotides of the present disclosure may be introduced into virtually any host cell of interest, including but not limited to bacteria, yeast, fungi, insects, plants, and animal cells using routine methods known to the skilled artisan.

The genus Escherichia includes Escherichia coli K12/DH1, Escherichia coli JM103, Escherichia coli JA221, Escherichia coli HB101, Escherichia coli C600 and the like. The genus Bacillus includes Bacillus subtilis MI 114, Bacillus subtilis 207-21, and the like.

Yeast useful for hosting the polynucleotides of the disclosure include Saccharomyces cerevisiae AH22, AH22 R⁻, NA87-11A, DKD-5D, 20B-12, Schizosaccharomyces pombe NCYC1913, NCYC2036, Pichia pastoris KM71, and the like.

Polynucleotides of the present disclosure may be introduced into insect cells using, for example, viral vectors, such as AcNPV. Insect host cells include any of the following cell lines: cabbage armyworm larva-derived established line (Spodoptera frugiperda cell; Sf cell), MG1 cells derived from the mid-intestine of Trichoplusiani, High Five, cells derived from an egg of Trichoplusiani, Mamestra brassicae-derived cells, Estigmena acraz-derived cells, and the like. When the virus is BmNPV, cells of a Bombyx mori-derived line (Bombyx mori N cell; BmN cell) and the like are used. Sf cells include, for example, Sf9 cells (ATCC CRL1711), Sf21s cells, and the like.

Mammalian cell lines may be used, including, without limitation monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells, human embryonic kidney (HEK) cells (e.g., HEK293, HEK293T), COS cells (e.g., COS1 or COS), BHK cells, MDCK cells, NS0 cells, PER. C6 cells, CRL7O3O cells, HsS78Bst cells, HeLa cells, NIH 3T3 cells, HepG2 cells, SP210 cells, R1.1 cells, B-W cells, L-M cells, BSC1 cells, BSC40 cells, YB/20 cells and BMT10 cells, and the like.

In certain embodiments, suitable cells are of a mammalian, a bacterial, or an insect origin. In certain embodiments, the cell is selected from the group consisting of a HEK293 cell, a HEK293T cells, a VERO cell, a BHK cell, and a BSR cell.

All the above-mentioned host cells may be haploid (monoploid), or polyploid (e.g., diploid, triploid, tetraploid and the like.

Various methods of introducing polynucleotides of the disclosure into a host cell described herein are known to those of ordinary skill in the art. For example, such methods may include the use of any transfection method known in the art (e.g., using lysozyme, PEG, CaCl2) coprecipitation, electroporation, microinjection, particle gun, lipofection, Agrobacterium and the like). The transfection method is selected based on the host cell to be transfected. Escherichia coli can be transformed according to the methods described in, for example, Proc. Natl. Acad. Sci. USA, 69, 2110 (1972), Gene, 17, 107 (1982) and the like. Methods for transducing the genus Bacillus are described in, for example, Molecular & General Genetics, 168, 111 (1979). Yeast cells are transduced using methods described in, for example, Methods in Enzymology, 194, 182-187 (1991), Proc. Natl. Acad. Sci. USA, 75, 1929 (1978) and the like. Insect cells are transfected using methods described in, for example, Bio/Technology, 6, 47-55 (1988) and the like. Mammalian cells are transfected using methods described in, for example, Cell Engineering additional volume 8, New Cell Engineering Experiment Protocol, 263-267 (1995) (published by Shujunsha), and Virology, 52, 456 (1973).

Cells comprising expression vectors of the present disclosure are cultured according to known methods, which vary depending on the host. For example, when Escherichia coli or genus Bacillus cells are cultured, a liquid medium is used. The medium preferably contains a carbon source, nitrogen source, inorganic substance and other components necessary for the growth of the transformant. Examples of the carbon source include glucose, dextrin, soluble starch, sucrose, and the like; examples of the nitrogen source include inorganic or organic substances such as ammonium salts, nitrate salts, corn steep liquor, peptone, casein, meat extract, soybean cake, potato extract, and the like; and examples of the inorganic substance include calcium chloride, sodium dihydrogen phosphate, magnesium chloride, and the like. The medium may also contain yeast extract, vitamins, growth promoting factors, and the like. The pH of the medium is preferably between about 5 to about 8. As a medium for culturing Escherichia coli, for example, M9 medium containing glucose and casamino acid (see, e.g., Journal of Experiments in Molecular Genetics, 431-433, Cold Spring Harbor Laboratory, New York 1972) is used. Escherichia coli is cultured at generally about 15 to about 43° C. Where necessary, aeration and stirring may be performed. The genus Bacillus is cultured at generally about 30 to about 40° C. Where necessary, aeration and stirring is performed.

Examples of medium suitable for culturing yeast include Burkholder minimum medium, SD medium containing 0.5% casamino acid, and the like. The pH of the medium is preferably about 5-about 8. The culture is performed at generally about 20° C. to about 35° C. Where necessary, aeration and stirring may be performed.

As a medium for culturing an insect cell or insect, Grace's Insect Medium containing an additive such as inactivated 10% bovine serum, and the like are used. The pH of the medium is preferably about 6.2 to about 6.4. Cells are cultured at about 27° C. Where necessary, aeration and stirring may be performed.

Mammalian cells are cultured, for example, in any one of minimum essential medium (MEM) containing about 5 to about 20% of fetal bovine serum, Dulbecco's modified Eagle medium (DMEM), RPMI 1640 medium, 199 medium, and the like. The pH of the medium is preferably about 6 to about 8. The culture is performed at about 30° C. to about 40° C. Where necessary, aeration and stirring may be performed.

### I. PACKAGING SYSTEMS AND METHODS THEREOF

The present disclosure provides packaging systems useful for the recombinant preparation of a rabies virus particle described herein. In particular, the packaging systems provide necessary components required for the preparation of a rabies virus particle described herein. In certain embodiments, the packaging system is useful for the recombinant preparation of a rabies virus particle comprising a recombinant rabies virus genome, wherein the genome lacks a G gene encoding for a rabies virus glycoprotein or a functional variant thereof; and/or the genome lacks an L gene encoding for a rabies virus polymerase or a functional variant thereof. In certain embodiments, the packaging system is useful for the recombinant preparation of a rabies virus particle comprising a recombinant rabies virus genome, wherein the genome lacks a G gene encoding for a rabies virus glycoprotein or a functional variant thereof. In certain embodiments, the packaging system is useful for the recombinant preparation of a rabies virus particle comprising a recombinant rabies virus genome, wherein the genome lacks a G gene encoding for a rabies virus glycoprotein or a functional variant thereof; and the genome lacks an L gene encoding for a rabies virus polymerase or a functional variant thereof.

The packaging systems described herein generally comprise or consist of: (i) an N gene encoding for a rabies virus nucleoprotein or a functional variant thereof; (ii) a P gene encoding for a rabies virus phosphoprotein or a functional variant thereof; and (iii) an L gene encoding for a rabies virus polymerase or a functional variant thereof. In certain embodiments, the packaging system further comprises an M gene encoding for a rabies virus matrix protein or a functional variant thereof. In certain embodiments, the packaging system further comprises a G gene encoding for a rabies virus glycoprotein or a functional variant thereof.

The N, P, and L genes of the packaging system can be provided in one or more vectors (e.g., transfecting plasmids). For example, the packaging system can comprise a separate transfecting plasmid for each of the N, P, and L genes, e.g., a first transfecting plasmid comprising an N gene encoding for a rabies virus nucleoprotein or a functional variant thereof; a second transfecting plasmid comprising a P gene encoding for a rabies virus phosphoprotein or a functional variant thereof; and a third transfecting plasmid comprising an L gene encoding for a rabies virus polymerase or a functional variant thereof. In certain embodiments, a single transfecting plasmid comprises two or more of the N, P, and L genes. For example, the packaging system can comprise a transfecting plasmid comprising an N gene encoding for a rabies virus nucleoprotein or a functional variant thereof, and a P gene encoding for a rabies virus phosphoprotein or a functional variant thereof; the packaging system can comprise a transfecting plasmid comprising an N gene encoding for a rabies virus nucleoprotein or a functional variant thereof, and an L gene encoding for a rabies virus polymerase or a functional variant thereof; the packaging system can comprise a transfecting plasmid comprising a P gene encoding for a rabies virus phosphoprotein or a functional variant thereof, and an L gene encoding for a rabies virus polymerase or a functional variant thereof. In certain embodiments, the packaging system can comprise a transfecting plasmid comprising an N gene encoding for a rabies virus nucleoprotein or a functional variant thereof, a P gene encoding for a rabies virus phosphoprotein or a functional variant thereof, and an L gene encoding for a rabies virus polymerase or a functional variant thereof.

The M and G genes of the packaging system can be provided in one or more transfecting plasmids. In certain embodiments, the packaging system comprises a separate transfecting plasmid for the M and G genes. For example, in certain embodiments, the packaging system can further comprise a transfecting plasmid comprising an M gene encoding for a rabies virus matrix protein or a functional variant thereof. In certain embodiments, the packaging system can further comprise a transfecting plasmid comprising a G gene encoding for a rabies virus glycoprotein or a functional variant thereof. The M and/or G gene can also be combined into a transfecting plasmid that comprises a N, P, and/or L gene as described herein. For example, a single transfecting plasmid can comprise an N gene encoding for a rabies virus nucleoprotein or a functional variant thereof, a P gene encoding for a rabies virus phosphoprotein or a functional variant thereof, an L gene encoding for a rabies virus polymerase or a functional variant thereof, an M gene encoding for a rabies virus matrix protein or a functional variant thereof, and a G gene encoding for a rabies virus glycoprotein or a functional variant thereof. Various other combinations can readily be appreciated by those of ordinary skill in the art.

The N, P, L, M, and/or G genes can all be under control of one or more transcriptional regulatory elements. In certain embodiments, the transcriptional regulatory element comprises a promoter and/or enhancer sequence. In certain embodiments, the transcriptional regulatory element comprises an EF1α promoter. Various promoters and/or enhancer sequences are known in the art and are described herein as examples, and one of ordinary skill in the art would be able to select a suitable promoter and/or enhancer sequence for their needs.

Where two or more of the N, P, L, M, and/or G genes reside on the same vector, the two or more genes may be present in one or more expression cassettes. For example, each of the N, P, L, M, and/or G genes can be within their own expression cassette each comprising a transcriptional regulatory element and/or transcriptional termination element.

Where two or more genes reside in the same expression cassette, the genes may be separated by a linker sequence. In certain embodiments, the linker sequence is a ribosomal skipping element comprising a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). In certain embodiments, the linker sequence is a ribosomal skipping element comprising a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAV0, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate linker sequence for their needs.

In certain embodiments, a single vector (e.g., transfecting plasmid) comprises a first expression cassette comprising the N and P genes, and a second expression cassette comprising the L gene. In certain embodiments, the first expression cassette comprises from 5′ to 3′: a transcriptional regulatory element; the P gene; and the N gene. In certain embodiments, the first expression cassette comprises from 5′ to 3′: a transcriptional regulatory element; the P gene; a ribosomal skipping element; and the N gene. In certain embodiments, the second expression cassette comprises from 5′ to 3′: a transcriptional regulatory element; and the L gene. In certain embodiments, the first expression cassette and the second expression cassette can be in the same orientation within the vector. In certain embodiments, the first expression cassette and the second expression cassette can be in the opposite orientation within the vector.

Accordingly, a packaging system of the present disclosure comprises: (i) a recombinant rabies virus genome vector (e.g., virus genome transfecting plasmid); and (ii) one or more transfecting plasmids comprising the N, P, L, M, and/or G genes. The one or more transfecting plasmids comprising the N, P, L, M, and/or G genes can be introduced into a host cell (e.g., a recombinant rabies virus particle packaging cell) using various methods known to those of ordinary skill in the art. For example, the one or more transfecting plasmids can be introduced into a suitable host cell by electroporation, nucleofection, or lipofection.

The present disclosure also provides a method for the recombinant preparation of a rabies virus particle, wherein the method comprises introducing a packaging system described herein into a cell under conditions operative for enveloping the recombinant rabies virus genome to form the recombinant rabies virus particle. In certain embodiments, host packaging cell can be transiently transfected with the one or more transfecting plasmids comprising the N, P, L, M, and/or G genes. In certain embodiments, the host packaging cell can be transfected with the one or more transfecting plasmids comprising the N, P, L, M, and/or G genes, wherein the host packaging cell is further made into a stable cell line. Various methods for producing stable cell lines are known to those of ordinary skill in the art. In general, the gene of interest (e.g., N, P, L, M and/or G genes) is introduced into a cell, and then into the nucleus of the cell, and finally integrated into the genome of the cell. Chromosomal integration events are rare and stably-integrated cell lines have to be selected and cultured. Various selection systems are known in the art, including resistance to antibiotics such as neomycin phosphotransferase, conferring resistance to G418, dihydrofolate reductase (DHFR), or glutamine synthetase. Other methods for producing stable cell lines include the use of the Sleeping Beauty (SB) system, as described in the Experimental Examples. Briefly, a transposon comprising the integrant of interest is designed with flanking inverted repeat/direct repeat sequences that result in precise integration into a TA dinucleotide. Methods for SB transposon based stable cell line generation is known in the art, see, e.g., Davidson et al., Cold Spring Harb Protoc. (2009) 4(8): 1018-1023. Stable cell lines can also be generated via the use of lentiviral vectors, see, e.g., Tandon et al., Bio Protoc. (2018) 8(21): e3073.

A recombinant rabies virus genome vector (e.g., virus genome transfecting plasmid) is then introduced into a host packaging cell that has the N, P, L, M, and/or G genes stably-integrated or transiently transfected therein.

As such, in certain embodiments, a method for the recombinant preparation of a rabies virus particle comprises introducing (i) a recombinant rabies virus genome vector (e.g., virus genome transfecting plasmid); and (ii) one or more transfecting plasmids comprising the N, P, L, M, and/or G genes into a host packaging cell. In certain embodiments, a method for the recombinant preparation of a rabies virus particle comprises introducing a recombinant rabies virus genome vector (e.g., virus genome transfecting plasmid) into a host packaging cell, wherein the host packaging cell comprises the N, P, L, M, and/or G genes stably integrated therein. Methods for the preparation of recombinant rabies virus particles are known in the art, see, e.g., Trabelsi et al., Vaccine (2019) 37(47): 7052-7060; Wickersham et al., Nature Protoc. (2010) 5(3): 595-606; Ghanem et al., Eur. J. Cell Biol. (2012) 91: 10-16; Osakada and Wickersham, Nature Protoc. (2013) 8(8): 1583-1601; and Sullivan and Wickersham, Cold Spring Harb Protoc. (2015) 4: 386-91, the disclosures of which are herein incorporated by reference in their entireties.

In certain embodiments, the recombinant rabies virus particle titer that is obtained using a method of production described herein is greater than about 1E8 transducing units (TU)/mL. For example, in certain embodiments, the recombinant rabies virus particle titer that is obtained is about 8E7 TU/mL, about 9E7 TU/mL, about 1E8 TU/mL, about 1.1E8 TU/mL, about 1.2E8 TU/mL, about 1.3E8 TU/mL, about 1.4E8 TU/mL, about 1.5E8 TU/mL, about 1.6E8 TU/mL, about 1.7E8 TU/mL, about 1.8E8 TU/mL, about 1.9E8 TU/mL, about 2E8 TU/mL, about 2.5E8 TU/mL, about 3E8 TU/mL, about 3.5E8 TU/mL, about 4E8 TU/mL, about 4.5E8 TU/mL, about 5E8 TU/mL, about 5.5E8 TU/mL, about 6E8 TU/mL, about 6.5E8 TU/mL, about 7E8 TU/mL, about 7.5E8 TU/mL, about 8E8 TU/mL, about 8.5E8 TU/mL, about 9E8 TU/mL, about 9.1E8 TU/mL, about 9.2E8 TU/mL, about 9.3E8 TU/mL, about 9.4E8 TU/mL, about 9.5E8 TU/mL, about 9.6E8 TU/mL, about 9.7E8 TU/mL, about 9.8E8 TU/mL, about 9.9E8 TU/mL, about 1E9 TU/mL, about 1.1E9 TU/mL, about 1.2E9 TU/mL, or any value in between the aforementioned titers. In certain embodiments, the recombinant rabies virus particle titer that is obtained is from about 1E8 TU/mL to about 1E9 TU/mL, e.g., from 8E7 TU/mL to 1.2E9 TU/mL, and any range therebetween.

### J. METHODS OF GENE THERAPY

Provided herein are methods of gene therapy using the recombinant rabies virus particles described herein. In certain embodiments, a method for expressing a theapeutic transgene in a target cell, is provided. In certain embodiments, a method for expressing a base editor in a target cell, is provided.

In certain embodiments, a method for expressing a therapeutic transgene in a target cell comprises tranducing a target cell with a recombinant rabies virus particle as described herein. For example, a method for expressing a therapeutic transgene in a target cell comprises transducing a target cell with a recombinant rabies virus particle comprising a rabies virus glycoprotein; and a recombinant rabies virus genome comprising a nucleic acid encoding a therapeutic transgene, wherein: the genome lacks a G gene encoding for a rabies virus glycoprotein or a functional variant thereof; and/or the genome lacks an L gene encoding for a rabies virus polymerase or a functional variant thereof. In certain embodiments, the method comprises transducing a target cell with a recombinant rabies virus particle comprising a rabies virus glycoprotein; and a recombinant rabies virus genome comprising a nucleic acid encoding a therapeutic transgene, wherein: the genome lacks a G gene encoding for a rabies virus glycoprotein or a functional variant thereof. In certain embodiments, the method comprises transducing a target cell with a recombinant rabies virus particle comprising a rabies virus glycoprotein; and a recombinant rabies virus genome comprising a nucleic acid encoding a therapeutic transgene, wherein: the genome lacks a G gene encoding for a rabies virus glycoprotein or a functional variant thereof; and the genome lacks an L gene encoding for a rabies virus polymerase or a functional variant thereof.

Various methods of transducing a target cell with a recombinant virus particle are known to those of ordinary skill in the art. For example, the target cell can be contacted with the recombinant virus particle, resulting in receptor-mediated attachment of the virus particle, followed by clathrin-dependent endocytosis of the virus particle into the cell.

In certain embodiments, methods are provided for expressing a nucleobase editor in a target cell. For example, such methods comprise transducing a target cell with a recombinant rabies virus particle, wherein the recombinant virus particle comprises: a rabies virus glycoprotein; and a recombinant rabies virus genome comprising a nucleic acid encoding a nucleobase editor comprising a polynucleotide programmable nucleotide binding domain and a nucleobase editing domain, wherein: the genome lacks a G gene encoding for a rabies virus glycoprotein or a functional variant thereof; and/or the genome lacks an L gene encoding for a rabies virus polymerase or a functional variant thereof. In certain embodiments, the method comprises transducing a target cell with a recombinant rabies virus particle, wherein the recombinant virus particle comprises: a rabies virus glycoprotein; and a recombinant rabies virus genome comprising a nucleic acid encoding a nucleobase editor comprising a polynucleotide programmable nucleotide binding domain and a nucleobase editing domain, wherein: the genome lacks a G gene encoding for a rabies virus glycoprotein or a functional variant thereof. In certain embodiments, the method comprises transducing a target cell with a recombinant rabies virus particle, wherein the recombinant virus particle comprises: a rabies virus glycoprotein; and a recombinant rabies virus genome comprising a nucleic acid encoding a nucleobase editor comprising a polynucleotide programmable nucleotide binding domain and a nucleobase editing domain, wherein: the genome lacks a G gene encoding for a rabies virus glycoprotein or a functional variant thereof; and the genome lacks an L gene encoding for a rabies virus polymerase or a functional variant thereof.

Where the methods are for expressing a nucleobase editor in a target cell, the polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

In certain embodiments, the gRNA is provided to the target cell in cis. For example, the gRNA can be comprised within the recombinant rabies virus genome. The gRNA can be comprised within the recombinant rabies virus genome at any location, for example, between a one or more rabies virus genes (e.g., an N gene or a P gene) and the nucleic acid encoding the nucleobase editor, or between two rabies virus genes, or at a terminal end of the recombinant rabies virus genome (e.g., the 5′ end, or the 3′ end).

In certain embodiments, the gRNA is provided to the target cell in trans (e.g., provided exogenously). For example, the gRNA can be comprises within a separate vector outside of the recombinant rabies virus particle. Suitable vectors include, without limitation, viral vectors, plasmids, and other known to those of skill in the art. In embodiments where the gRNA is provided to the target cell in trans, the gRNA vector is introduced into the target cell via various methods known to those of skill in the art, for example, without limitation, electroporation.

Methods for delivering a therapeutic transgene (e.g., a nucleobase editor) to a subject are also provided. In certain embodiments, the method comprises administering to the subject a recombinant rabies virus particle, wherein the recombinant virus particle comprises: a rabies virus glycoprotein; and a recombinant rabies virus genome comprising a nucleic acid encoding the therapeutic transgene (e.g., a nucleobase editor comprising a polynucleotide programmable nucleotide binding domain and a nucleobase editing domain), wherein: the genome lacks a G gene encoding for a rabies virus glycoprotein or a functional variant thereof; and/or the genome lacks an L gene encoding for a rabies virus polymerase or a functional variant thereof. In certain embodiments, the method comprises administering to the subject a recombinant rabies virus particle, wherein the recombinant virus particle comprises: a rabies virus glycoprotein; and a recombinant rabies virus genome comprising a nucleic acid encoding the therapeutic transgene (e.g., a nucleobase editor comprising a polynucleotide programmable nucleotide binding domain and a nucleobase editing domain), wherein: the genome lacks a G gene encoding for a rabies virus glycoprotein or a functional variant thereof. In certain embodiments, the method comprises administering to the subject a recombinant rabies virus particle, wherein the recombinant virus particle comprises: a rabies virus glycoprotein; and a recombinant rabies virus genome comprising a nucleic acid encoding the therapeutic transgene (e.g., a nucleobase editor comprising a polynucleotide programmable nucleotide binding domain and a nucleobase editing domain), wherein: the genome lacks a G gene encoding for a rabies virus glycoprotein or a functional variant thereof; and the genome lacks an L gene encoding for a rabies virus polymerase or a functional variant thereof.

The methods of delivery and/or expressing a therapeutic transgene (e.g., a nucleobase editor comprising a polynucleotide programmable nucleotide binding domain and a nucleobase editing domain) find use in the treatment of a disease or disorder. In certain embodiments, a method of treating a disease or disorder in a subject comprises administering a recombinant rabies virus particle described herein, or a pharmaceutical composition described herein. In certain embodiments, the disease or disorder is a neurologic disease or disorder. In certain embodiments, the disease or disorder is a ophthalmic disease or disorder.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### K. EXPERIMENTAL EXAMPLES

### Example 1: Generation of Stable Cell Lines

The stable cell lines described in Table 16 below were generated:

TABLE 16
Stable cell lines

| | | Integrating | Selection |
|---|---|---|---|
| Cell line name | Description | vector | marker |
| CA1.9 | HEK293T cell line | VIR120 | Blasticidin |
| (RABV-G) | stably expressing | | |
| | rabies virus G gene | | |
| CE1.13 | HEK293T cell line | VIR035 | Blasticidin |
| (RABV-G) | stably expressing | | |
| | codon-optimized rabies | | |
| | virus G gene | | |
| CA3.11 | HEK293T cell line | VIR069 | Blasticidin |
| | stably expressing | | |
| | rabies virus N, P, and L | | |
| | genes | | |
| CA4.27 | HEK293T cell line | VIR071 | Zeocin |
| | stably expressing | | |
| | rabies virus N, P, and L | | |
| | genes | | |
| CA3.10 and | HEK293T cell line | VIR121 | Blasticidin |
| CE1.10 | stably expressing | | |
| | rabies virus M gene | | |
| CE1.30 | HEK293T cell line | VIR112 | Zeocin |
| | stably expressing | | |
| | rabies virus N, P, M, | | |
| | and L genes | | |

One of the Sleeping Beauty transposase system-compatiable integrating vectors selected from VIR120, VIR035, VIR069, VIR071, VIR121, and VIR112 were co-transfected into HEK293T cells with the Sleeping Beauty transposase SB100X. VIR120 contains an expression cassette comprising a rabies virus G gene under the control of an EF1-alpha promoter; VIR035 contains an expression cassette comprising a codon-optimized rabies virus G gene under the control of an EF1-alpha promoter; VIR069 contains an expression cassette comprising from 5′ to 3′: an EF1-alpha promoter, a rabies virus N gene, a T2A peptide, a rabies virus P gene, a P2A peptide, and a rabies virus L gene; VIR071 contains a first expression cassette comprising from 5′ to 3′: an EF1-alpha promoter, a rabies virus M gene, a P2A peptide, a rabies virus P gene, an IRES, and a rabies virus N gene, and a second expression cassette comprising from 5′ to 3′: an RPBSA promoter, and a rabies virus L gene, wherein the first and the second expression cassettes are in opposite orientations; VIR121 contains an expression cassette comprising a rabies virus M gene under the control of an EF1-alpha promoter; and VIR112 contains an expression cassette comprising from 5′ to 3′: a rabies N gene, an IRES, a rabies P gene, an IRES, and a rabies M gene under the control of an EF1-alpha, and and a second expression cassette comprising from 5′ to 3′: a Zeocin, T2A peptide, and rabies L gene under the control of an RPBSA promoter, wherein the first and the second expression cassettes are in opposite orientations. Schematics of the VIR069, VIR071, and VIR112 vectors are shown in FIG. 12.

One day after co-transfection, selection was begun using blasticidin or zeocin, depending on the integrating vector used. Selection continued through days 2 to 21 after co-transfection as necessary. By day 28, all surviving cells had the stably integrated transgene.

### Example 2: Production of Recombinant Rabies Virus Particles

For primary production, on day 0, Lipofectamine 3000 was used to transfect (i) 1.5 μg of complement plasmid mix of expression vectors, and (ii) 0.5 μg of plasmid encoding the rabies replicon, into a stable cell line. Transfections were performed according to Table 17:

TABLE 17
Transfection Mixes

| Stable cell line | Complement plasmid mix | Replicon |
|---|---|---|
| RABV-G | DNA52 + VIR8, VIR9, | VIR045 “G-deleted” |
| | VIR10, VIR11, VIR12 | |
| RABV-G | DNA52 + VIR8, VIR9, | VIR092 “G/L-deleted” |
| | VIR10, VIR11, VIR12 | |
| CA3.11 | DNA52 + VIR8, VIR9, | VIR045 “G-deleted” |
| | VIR10, VIR11, VIR12 | |
| CA3.11 | DNA52 + VIR8, VIR9, | VIR092 “G/L-deleted” |
| | VIR10, VIR11, VIR12 | |
| CA4.27 | DNA52 + VIR8, VIR9, | VIR045 “G-deleted” |
| | VIR10, VIR11, VIR12 | |
| CA4.27 | DNA52 + VIR8, VIR9, | VIR092 “G/L-deleted” |
| | VIR10, VIR11, VIR12 | |

The VIR045 replicon contains rabies SAD L16 full replicon with the G gene deleted. The VIR092 replicon was derived from VIR045 with the L gene further deleted. Both VIR045 and VIR092 contains sequence encoding GFP. DNA52 is an expression vector comprising a sequence encoding T7 RNA polymerase. VIR8-12 are an expression vectors comprising a rabies virus N, P, M, G, and L genes.

On day 1, media was changed to OptiMem+5% FBS (“O5”). Day 1 media was discarded. Beginning on day 3, viral supernatant was harvested and media was replaced with fresh O5 media daily. Viral supernatants from days 3-7 were pooled and stored at 4° C.

The pooled viral supernatants were clarified to remove cellular debris by centrifugation at 4000 rpm for 15 minutes. Viral particles were precipitated and concentrated following protocol for the Lenti-X Concentrator (Takara Bio). Supernatant was removed, and the pellet was resuspended in 05 media to produce concentrated viral stock. The concentrated viral stock was used to seed subsequent amplification passages.

Secondary viral amplification was performed as follows. On day 0, viral stock was added to stable cell lines. Additional plasmids were co-transfected into the stable cell lines at the time of transduction, if necessary. For viral stock produced using the VIR045 replicon, nothing additional was required when amplified in the RABV-G stable cell line. For viral stock produced using the VIR092 replicon, amplication was performed in the following ways, with efficiency shown in parenthesis—more “+” indicates higher efficiency: (1) RABV-G stable cell line co-transfected with a plasmid containing the N, P, and L genes (+); (2) CA4.27 stable cell line co-transfected with a plasmid containing the G gene (++); and (3) CA4.27 stable cell line with the G gene further stably integrated (+++).

On day 1, media was changed to O5 media. Day 1 media was discarded. On days 2 to 7, viral supernatants were harvested and pooled.

In another experiment, GFP expression was compared between primary transfection cell lines HEK293T control cells, RABV-G, CA3.11, and CA4.27, transfected with either the VIR045 or the VIR092 replicon. Full complement plasmid mixes were co-transfected into each cell line. Table 18 shows qualitative levels of GFP expression based on images taken 8 days after primary transfection, where the more “+” indicates higher GFP expression.

TABLE 18
GFP Expression in Primary Transfection Cell Lines

| Replicon | HEK293T | RABV-G | CA3.11 | CA4.27 |
|---|---|---|---|---|
| VIR045 | + | +++++ | + | +++ |
| VIR092 | − | − | − | ++ |

Viral supernatants were collected daily on days 2 to 4, pooled, and concentrated by the Lenti-X Concentrator. The concentrated VIR045 viral supernatant was added to RABV-G cells and the concentration VIR092 viral supernatant was added to RABV-G cells transfected with a plasmid containing the N, P, and L genes. Qualitative levels of GFP expression, indicating production of recombinant rabies virus particles, based on images taken 2 days after transfection are shown in Table 19, where the more “+” indicates higher GFP expression.

TABLE 19
GFP Expression in First Amplification

| Replicon | HEK293T | RABV-G | CA3.11 | CA4.27 |
|---|---|---|---|---|
| VIR045 | + | +++++ | + | + |
| VIR092 | +++ | +++ | +++ | +++ |

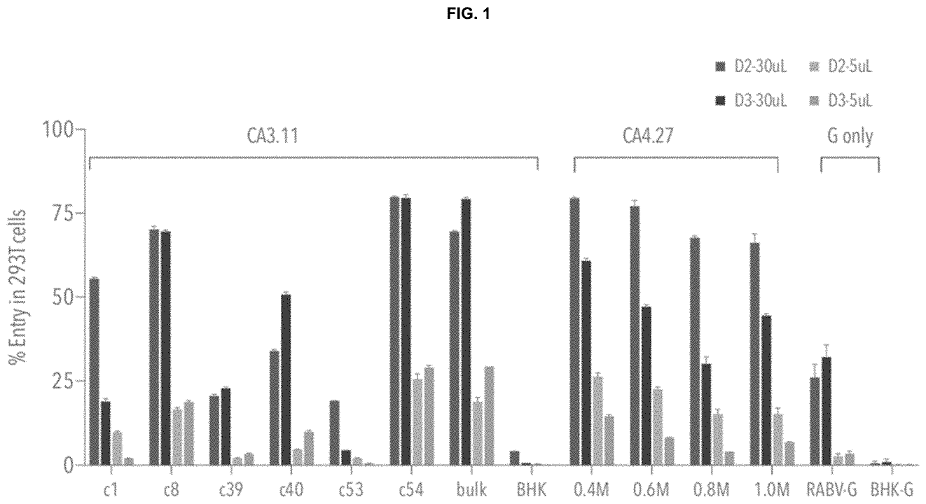

In another experiment, recombinant rabies virus relative infectivity was determined for the viral supernatant obtained from using various stable cell lines were determined (FIG. 1).

Stable cell lines c1, c8, c39, c40, c53, and c54 were clonal cell lines derived from the CA3.11 stable cell line (“bulk”). BHK cell lines using integrating vector VIR069 (“BHK”), and integrating vector VIR120 (“BHK-G”) were also generated. CA4.27 cells were plated at 0.4, 0.6, 0.8, or 1 million cells per well.

Viral supernatant was harvested on different days (D2 or D3) and subsequently used to infect naive HEK293T cells at the volumes indicates on FIG. 1 (5 uL or 30 uL). Titering was performed by flow cytometry, showing the percentage of cells that were infected as determined by expression of GFP.

### Example 3: Recombinant Rabies Virus Particle Gene Delivery

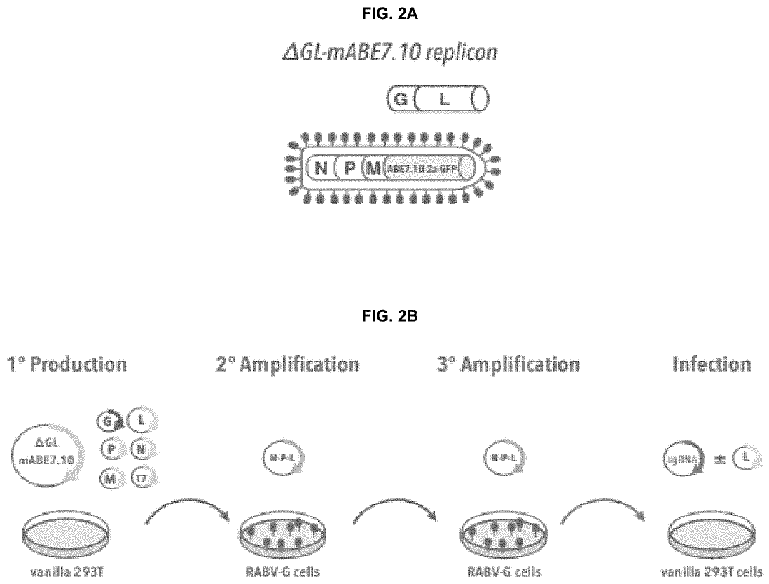

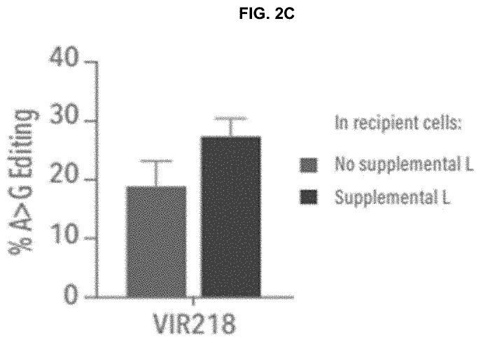

To investigate whether recombinant rabies virus particles could be used for gene delivery, replicon VIR218 was generated. VIR218 was derived from VIR092 with the addition of sequence encoding the adenosine deaminase ABE7.10; FIG. 2A is a schematic of VIR218. FIG. 2B is a schemating showing the production and amplification scheme that was followed. Primary production was performed by co-transfecting VIR218 with a full complement plasmid mix into naive HEK293T cells. Secondary and tertiary amplifications were performed with additional transfection of a plasmid containing the N, P, and L genes on the RABV-G cell line. Viral supernatants were collected and concentrated as described above to produce a viral stock. The viral stock was then added to naive 293T cells together with transfecting via lipofection a plasmid comprising a gRNA targeting HEK2-2 (gaacacaaagcatagactgc; SEQ ID NO:4011), and optionally co-transfecting with a plasmid comprising the L gene (“supplemental L”). Genomic DNA was extracted and standard PCR/library preparation was performed to amplify out the genomic target and assess editing (FIG. 2C). As shown in FIG. 2C, A>G editing was detected in infected HEK293T cells.

### Example 4: Recombinant Rabies Virus Genome

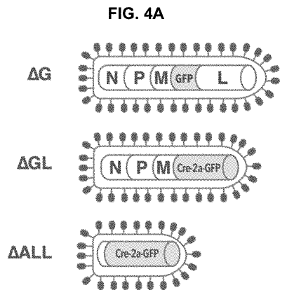

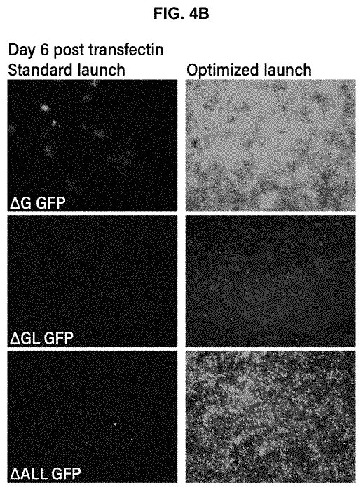

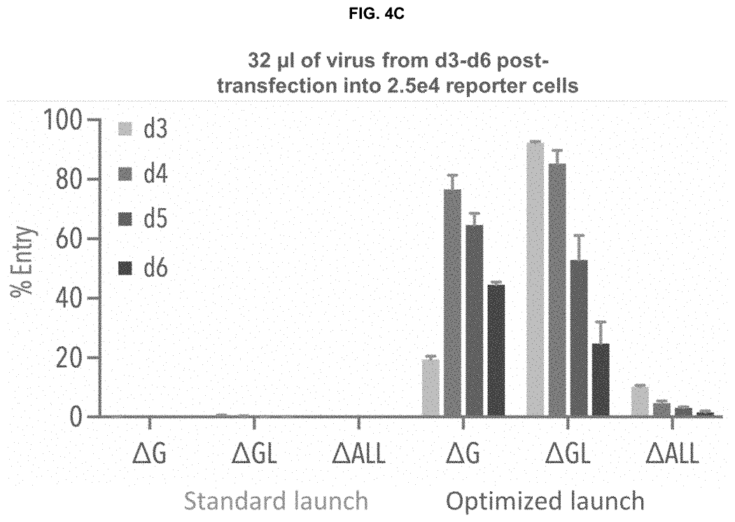

Examples 1-3 described the use of a recombinant rabies virus genome in a ΔGL background. Production of virus was next tested in a recombinant rabies virus genome in a ΔLL (i.e., ΔGLNPM) background (see depictions of the replicons in FIG. 4A). The tested rabies virus replicons encoded a Cre recombinase-2A-GFP reporter gene.

As shown in FIG. 4B, cells transfected with rabies virus vectors in a ΔG, ΔGL, or ΔLL (i.e., ΔGLNPM) background emitted flourescence from the Cre recombinase-2A-GFP reporter gene. Images were taken 6 days post-transfection under a standard transfection and an optimized transfection as depicted in FIG. 2B.

As shown in FIG. 4C, % viral entry of rabies virus produced in FIG. 4B was determined following transduction into reporter cells, as measured by GFP flourescence.

This work demonstrates that an engineered recombinant rabies virus genome that lacks all viral genes (i.e., ΔLL or ΔGLNPM) can be used to express a transgene.

### Example 5: Encoding gRNA into Rabies Genome with Cleaving tRNAs

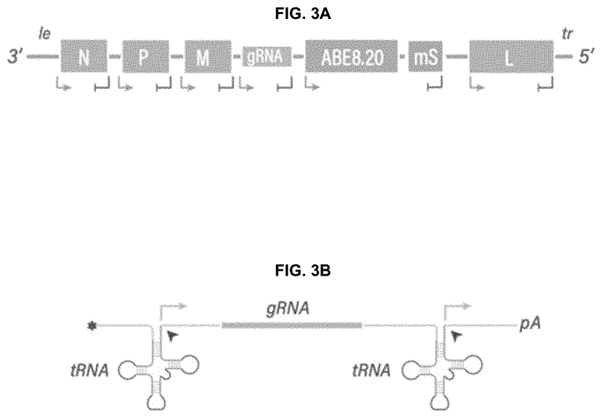

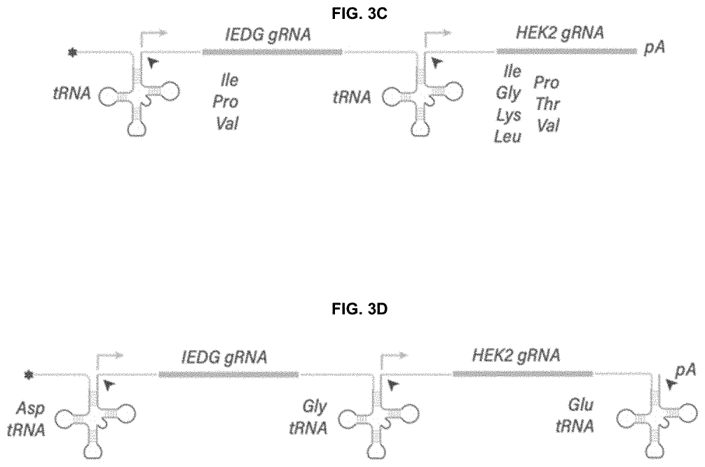

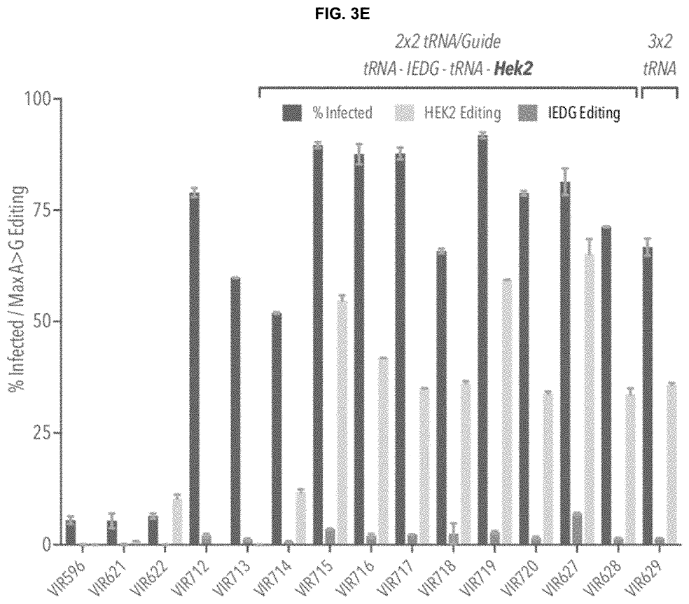

To investigate whether gRNA could be encoded in the rabies viral genome, replicon VIR621 was generated in the organization shown in FIG. 3A. VIR621 was derived from DNA538 which encoded two flanking cleaving tRNAs and an intervening gRNA (FIG. 3B) with the addition of sequences encoding the polynucleotide programmable nucleotide binding domain and adenosine deaminase contained in ABE8 and the viral genome lacking the G gene (FIG. 3A). Multiple target tRNAs were also encoded between or after different tRNA combinations allowing for multiplexing (FIG. 3C, FIG. 3D). Several combinations of tRNAs and gRNAs as listed in Table 20 were tested for editing efficiency in FIG. 3E. As shown in FIG. 3E, A>G editing of HEK2 and IEDG genes was detected in infected HEK293T cells with viral replicons containing no gRNA (VIR596), single gRNA targeting HEK2 (VIR621, VIR622), single gRNA targeting IEDG (VIR712, VIR713), or multiplexed multiple gRNAs targeting HEK2 and IEDG in the same viral replicon (VIR714, VIR715, VIR717, VIR718, VIR719, VIR720, VIR627, VIR628, VIR629).

TABLE 20
tRNA and gRNA Replicons

| | | | | |
|---|---|---|---|---|
| VIR621 | SynV ΔG | tRNA Pro | gtacaagTAAGAAGTTGAAT | 4024 |
| | ABE8-20- | 3′ | AACAAAATGCCGGAAATCTA | |
| | 2a-GFP | release | CGGATTGTGTATATCCATCA | |
| | tRNA | | TGAAAAAAACTAACACCCCT | |
| | Pro- | | CCTTTCGAACCATCCCAAAC | |
| | Hek2 | | ggctcgttggtctaggggta | |
| | gRNA | | tgattctcgcttagggtgcg | |
| | | | agaggtcccgggttcaaatc | |
| | | | ccggacgagcccGGAACACA | |
| | | | AAGCATAGACTGCgttttag | |
| | | | agctaGAAAtagcaagttaa | |
| | | | aataaggctagtccgttatc | |
| | | | aacttgaaaaagtggcaccg | |
| | | | agtcggtgcttttCGAGGAA | |
| | | | GGAGGTCTGAGGAGGTCACT | |
| | | | Gcgaaccagtttgtgtcggc | |
| | | | tcgttggtctaggggtatga | |
| | | | ttctcgcttagggtgcgaga | |
| | | | ggtcccgggttcaaatcccg | |
| | | | gacgagccctctagaagtgc | |
| | | | tgggtcatcta | |
| VIR622 | SynV ΔG | tRNA Ile | gtacaagTAAGAAGTTGAAT | 4025 |
| | ABE8-20- | 3′ | AACAAAATGCCGGAAATCTA | |
| | 2a-GFP | release | CGGATTGTGTATATCCATCA | |
| | tRNA | | TGAAAAAAACTAACACCCCT | |
| | Ile- | | CCTTTCGAACCATCCCAAAC | |
| | Hek2 | | gctccagtggcgcaatcggt | |
| | gRNA | | tagcgcgcggtacttataag | |
| | | | acagtgcacctgtgagcaat | |
| | | | gccgaggttgtgagttcaag | |
| | | | cctcacctggagcaGGAACA | |
| | | | CAAAGCATAGACTGCgtttt | |
| | | | agagctaGAAAtagcaagtt | |
| | | | aaaataaggctagtccgtta | |
| | | | tcaacttgaaaaagtggcac | |
| | | | cgagtcggtgcttCACACAC | |
| | | | ACAAgctccagtggcgcaat | |
| | | | cggttagcgcgcggtactta | |
| | | | taagacagtgcaGCCgCGAG | |
| | | | GAAGGAGGTCTGAGGAGGTC | |
| | | | ACTGcGGCcctgtgagcaat | |
| | | | gccgaggttgtgagttcaag | |
| | | | cctcacctggagcata | |
| VIR623 | SynV ΔG | tRNA | gtacaagTAAGAAGTTGAAT | 4026 |
| | ABE8-20- | apical | AACAAAATGCCGGAAATCTA | |
| | 2a-GFP | release | CGGATTGTGTATATCCATCA | |
| | tRNA | | TGAAAAAAACTAACACCCCT | |
| | Ile- | | CCTTTCGAACCATCCCAAAC | |
| | apical | | gctccagtggcgcaatcggt | |
| | Hek2 | | tagcgcgcggtacttataag | |
| | gRNA | | acagtgcaGAACACAAAGCA | |
| | | | TAGACTGCgttttagagcta | |
| | | | CCGAAAGGtagcaagttaaa | |
| | | | ataaggctagtccgttatca | |
| | | | acttgaaaaagtggcaccga | |
| | | | gtcggtgcttcacacacaca | |
| | | | caCGAGGAAGGAGGTCTGAG | |
| | | | GAGGTCACTGcgcctgtgag | |
| | | | caatgccgaggttgtgagtt | |
| | | | caagcctcacctggagcata | |
| VIR624 | SynV ΔG | tRNA | gtacaagTAAGAAGTTGAAT | 4027 |
| | ABE8-20- | apical | AACAAAATGCCGGAAATCTA | |
| | 2a-GFP | release | CGGATTGTGTATATCCATCA | |
| | tRNA | with | TGAAAAAAACTAACACCCCT | |
| | Ile- | long | CCTTTCGAACCATCCCAAAC | |
| | apical | linker | gctccagtggcgcaatcggt | |
| | Hek2 | | tagcgcgcggtacttataag | |
| | gRNA | | acagtgcagGAACACAAAGC | |
| | with | | ATAGACTGCgttttagagct | |
| | long | | aCCGAAAGGtagcaagttaa | |
| | linker | | aaCaaggctagtccgttatc | |
| | | | aacttgaaaaagtggcaccg | |
| | | | agtcggtgctttGGCCCGAG | |
| | | | GAAGGAGGTCTGAGGAGGTC | |
| | | | ACTGGGCCAAAACAACAACC | |
| | | | CAACCAACAAACCAACACCA | |
| | | | AACAACAAACCAAACCCCAA | |
| | | | CAAACAACCACCAACCCAAA | |
| | | | CAAcctgtgagcaatgccga | |
| | | | ggttgtgagttcaagcctca | |
| | | | cctggagcata | |
| VIR625 | SynV ΔG | tRNA | gtacaagTAAGAAGTTGAAT | 4028 |
| | ABE8-20- | apical | AACAAAATGCCGGAAATCTA | |
| | 2a-GFP | release | CGGATTGTGTATATCCATCA | |
| | tRNA | stabi- | TGAAAAAAACTAACACCCCT | |
| | Ile- | lized | CCTTTCGAACCATCCCAAAC | |
| | apical | | gctccagtggcgcaatcggt | |
| | Flek2 | | tagcgcgcggtacttataag | |
| | gRNA | | acagtgcaGGAGCCCGAACA | |
| | with | | CAAAGCATAGACTGCgtttt | |
| | long | | agagctaGGCCCGAGGAAGG | |
| | linker | | AGGTCTGAGGAGGTCACTGG | |
| | | | GCCtagcaagttaaaataag | |
| | | | gctagtccgttatcaacttg | |
| | | | aaaaagtggcaccgagtcgg | |
| | | | tgcttAAAACAACAACCCAA | |
| | | | CCAACAAACCAACACCAAAC | |
| | | | AACAAACCAAACCCCAACAA | |
| | | | ACAACCACCAACCCAAACAA | |
| | | | GGGCTCCcctgtgagcaatg | |
| | | | ccgaggttgtgagttcaagc | |
| | | | ctcacctggagcata | |
| VIR626 | SynV ΔG | tRNA Ile | gtacaagTAAGAAGTTGAAT | 4029 |
| | ABE8- | permuted | AACAAAATGCCGGAAATCTA | |
| | 20- | | CGGATTGTGTATATCCATCA | |
| | 2a-GFP | | TGAAAAAAACTAACACCCCT | |
| | tRNA Ile | | CCTTTCGAACCATCCCAAAC | |
| | permuted | | GGGCTCCcctgtgagcaatg | |
| | Hek2 | | ccgaggttgtgagttcaagc | |
| | gRNA | | ctcacctggagcaGAAAgct | |
| | | | ccagtggcgcaatcggttag | |
| | | | cgcgcggtacttataagaca | |
| | | | gtgcaGGAGCCCGAACACAA | |
| | | | AGCATAGACTGCgttttaga | |
| | | | gctaGGCCCGAGGAAGGAGG | |
| | | | TCTGAGGAGGTCACTGGGCC | |
| | | | tagc | |
| | | | aagttaaaataaggctagtc | |
| | | | cgttatcaacttgaaaaagt | |
| | | | ggcaccgagtcggtgcttAA | |
| | | | AACAACAACCCAACCAACAA | |
| | | | ACCAACACCAAACAACAAAC | |
| | | | CAAACCCCAACAAACAACCA | |
| | | | CCAACCCAAACAAta | |
| VIR627 | SynRV | P-IEDG- | AACATCCCTCAAAagactca | 4030 |
| | tRNA-Pro- | T- | aggaaagggctcgttggtct | |
| | Thr IEDG | Hek2 | aggggtatgattctcgctta | |
| | Hek2 AG | | gggtgcgagaggtcccgggt | |
| | Abe820m- | | tcaaatcccggacgagcccG | |
| | T2a- | | cgtGtAgggTaaccatgaac | |
| | mScarlet | | GTTTTAGAGCTAGAAATAGC | |
| | | | AAGTTAAAATAAGGCTAGTC | |
| | | | CGTTATCAACTTGAAAAAGT | |
| | | | GGCACCGAGTCGGTGCTTTT | |
| | | | TTCACACACACAAggctcca | |
| | | | tagctcaggggttagagcac | |
| | | | tggtcttgtaaaccaggggt | |
| | | | cgcgagttcaattctcgctg | |
| | | | gggcttGGAACACAAAGCAT | |
| | | | AGACTGCgttttagagctaG | |
| | | | CCgCGAGGAAGGAGGTCTGA | |
| | | | GGAGGTCACTGcGGCtagca | |
| | | | agttaaaataaggctagtcc | |
| | | | gttatcaacttgaaaaagtg | |
| | | | gcaccgagtcggtgcttttt | |
| | | | aaTTAAccgagaaaaaaa | |
| VIR628 | SynRV | V-IEDG- | AACATCCCTCAAAagactca | 4031 |
| | tRNA-Val- | K- | aggaaaggtttccgtagtgt | |
| | Lys IEDG | Hek2 | agtggttatcacgttcgcct | |
| | Hek2 AG | | cacacgcgaaaggtccccgg | |
| | Abe820m- | | ttcgaaaccgggcggaaaca | |
| | T2a- | | GcgtGtAgggTaaccatgaa | |
| | mScarlet | | cGTTTTAGAGCTAGAAATAG | |
| | | | CAAGTTAAAATAAGGCTAGT | |
| | | | CCGTTATCAACTTGAAAAAG | |
| | | | TGGCACCGAGTCGGTGCTTT | |
| | | | TTTCACACACACAAgcccgg | |
| | | | ctagctcagtcggtagagca | |
| | | | tgagactcttaatctcaggg | |
| | | | tcgtgggttcgagccccacg | |
| | | | ttgggcgGGAACACAAAGCA | |
| | | | TAGACTGCgttttagagcta | |
| | | | GCCgCGAGGAAGGAGGTCTG | |
| | | | AGGAGGTCACTGcGGCtagc | |
| | | | aagttaaaataaggctagtc | |
| | | | cgttatcaacttgaaaaagt | |
| | | | ggcaccgagtcggtgctttt | |
| | | | taaTTAAccgagaaaaaaa | |
| VIR629 | SynRV | D-IEDG- | AACATCCCTCAAAagactca | 4032 |
| | tRNA- | G- | aggaaagtcctcgttagtat | |
| | Asp- | Hek2-Q | agtggtgagtatccccgcct | |
| | Gly-Glu | | gtcacgcgggagaccggggt | |
| | IEDG | | tcgattccccgacggggagG | |
| | Hek2 | | cgtGtAgggTaaccatgaac | |
| | ΔG | | GTTTTAGAGCTAGAAATAGC | |
| | Abe820m- | | AA | |
| | T2a- | | GTTAAAATAAGGCTAGTCCG | |
| | mScarlet | | TTATCAACTTGAAAAAGTGG | |
| | | | CACCGAGTCGGTGCTTTTTT | |
| | | | CACACACACAAgcgttggtg | |
| | | | gtatagtggtgagcatagct | |
| | | | gccttccaagcagttgaccc | |
| | | | gggttcgattcccggccaac | |
| | | | gcaGG AACAC AAAGCAT | |
| | | | AG ACTGCgttttagagcta | |
| | | | GCCgCGAGGAAGGAGGTCTG | |
| | | | AGGAGGTCACTGcGGCtagc | |
| | | | aagttaaaataaggctagtc | |
| | | | cgttatcaacttgaaaaagt | |
| | | | ggcaccgagtcggtgctttC | |
| | | | ACACACACAAtccttggtgg | |
| | | | tctagtggttaggattcggc | |
| | | | gctctcaccgccgcggcccg | |
| | | | ggttcgattcccggtcaggg | |
| | | | aattaaTTAAccgagaaaaa | |
| | | | aa | |
| VIR712 | SynRV | VIR622 | AACATCCCTCAAAagactca | 4033 |
| | tRNA- | insert | aggaaaggctccagtggcgc | |
| | Ile- | | aatcggttagcgcgcggtac | |
| | Ile | | ttataagacagtgcacctgt | |
| | (corn) | | gagcaatgccgaggttgtga | |
| | IEDG | | gttcaagcctcacctggagc | |
| | Pad ΔG | | aGcgtGtAgggTaaccatga | |
| | Abe820m- | | acGTTTTAGAGCTAGAAATA | |
| | T2a- | | GCAAGTTAAAATAAGGCTAG | |
| | mScarlet | | TCCGTTATCAACTTGAAAAA | |
| | | | GTGGCACCGAGTCGGTGCTT | |
| | | | TTTTCACACACACAAgctcc | |
| | | | agtggcgcaatcggttagcg | |
| | | | cgcggtacttataagacagt | |
| | | | gcaGCCgCGAGGAAGGAGGT | |
| | | | CTGAGGAGGTCACTGcGGCc | |
| | | | ctgtgagcaatgccgaggtt | |
| | | | gtgagttcaagcctcacctg | |
| | | | gagcaTTAATTAAtccgaga | |
| | | | aaaaaa | |
| VIR713 | SynRV | 5′Ile | AACATCCCTCAAAagactca | 4034 |
| | tRNA-Ile | to Pad | aggaaaggctccagtggcgc | |
| | IEDG Pad | | aatcggttagcgcgcggtac | |
| | ΔG | | ttataagacagtgcacctgt | |
| | Abe820m- | | gagcaatgccgaggttgtga | |
| | T2a- | | gttcaagcctcacctggagc | |
| | mScarlet | | aGcgtGtAgggTaaccatga | |
| | | | acGTTTTAGAGCTAGAAATA | |
| | | | GCAAGTTAAAATAAGGCTAG | |
| | | | TCCGTTATCAACTTGAAAAA | |
| | | | GTGGCACCGAGTCGGT GCT | |
| | | | TTTTT aattaacgagaaaa | |
| | | | aaa | |
| VIR714 | SynRV | I-IEDG- | AACATCCCTCAAAagactca | 4035 |
| | tRNA- | I-Hek2 | aggaaaggctccagtggcgc | |
| | Ile-Ile | | aatcggttagcgcgcggtac | |
| | (corn) | | ttataagacagtgcacctgt | |
| | IEDG | | gagcaatgccgaggttgtga | |
| | Hek2 | | gttcaagcctcacctggagc | |
| | ΔG | | aGcgtGtAgggTaaccatga | |
| | Abe820m- | | acGTTTTAGAGCTAGAAATA | |
| | T2a- | | GCAAGTTAAAATAAGG | |
| | mScarlet | | CTAGTCCGTTATCAACTTGA | |
| | | | AAAAGTGGCACCGAGTCGGT | |
| | | | GCTTTTTTCACACACACAAg | |
| | | | ctccagtggcgcaatcggtt | |
| | | | agcgcgcggtacttataaga | |
| | | | cagtgcaGCCgCGAGGAAGG | |
| | | | AGGTCTGAGGAGGTCACTGc | |
| | | | GGCcctgtgagcaatgccga | |
| | | | ggttgtgagttcaagcctca | |
| | | | cctggagcaGGAACACAAAG | |
| | | | CATAGACTGCgttttagagc | |
| | | | taGCCgCGAGGAAGGAGGTC | |
| | | | TGAGGAGGTCACTGcGGCta | |
| | | | gcaagttaaaataaggctag | |
| | | | tccgttatcaacttgaaaaa | |
| | | | gtggcaccgagtcggtgctt | |
| | | | tttccgagaaaaaaa | |
| VIR715 | SynRV | I-IEDG- | AACATCCCTCAAAagactca | 4036 |
| | tRNA- | G-Hek2 | aggaaaggctccagtggcgc | |
| | Ile-Gly | | aatcggttagcgcgcggtac | |
| | IEDG | | ttataagacagtgcacctgt | |
| | Hek2 ΔG | | gagcaatgccgaggttgtga | |
| | Abe820m- | | gttcaagcctcacctggagc | |
| | T2a- | | aGcgtGtAgggTaaccatga | |
| | mScarlet | | acGTTTTAGAGCTAGAAATA | |
| | | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAgcgtt | |
| | | | ggtggtatagtggtgagcat | |
| | | | agctgccttccaagcagttg | |
| | | | acccgggttcgattcccggc | |
| | | | caacgcaGGAACACAAAGCA | |
| | | | TAGACTGCgttttagagcta | |
| | | | GCCgCGAGGAAGGAGGTCTG | |
| | | | AGGAGGTCACTGcGGCtagc | |
| | | | aagttaaaataaggctagtc | |
| | | | cgttatcaacttgaaaaagt | |
| | | | ggcaccgagtcggtgctttt | |
| | | | taaTTAAccgagaaaaaaa | |
| VIR716 | SynRV | I-IEDG- | AACATCCCTCAAAagactca | 4037 |
| | tRNA- | K-Hek2 | aggaaaggctccagtggcgc | |
| | Ile-Lys | | aatcggttagcgcgcggtac | |
| | IEDG | | ttataagacagtgcacctgt | |
| | Hek2 ΔG | | gagcaatgccgaggttgtga | |
| | Abe820m- | | gttcaagcctcacctggagc | |
| | T2a- | | aGcgtGtAgggTaaccatga | |
| | mScarlet | | acGTTTTAGAGCTAGAAATA | |
| | | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAgcccg | |
| | | | gctagctcagtcggtagagc | |
| | | | atgagactcttaatctcagg | |
| | | | gtcgtgggttcgagccccac | |
| | | | gttgggcgGGAACACAAAGC | |
| | | | ATAGACTGCgttttagagct | |
| | | | aGCCgCGAGGAAGGAGGTCT | |
| | | | GAGGAGGTCACTGcGGCtag | |
| | | | caagttaaaataaggctagt | |
| | | | ccgttatcaacttgaaaaag | |
| | | | tggcaccgagtcggtgcttt | |
| | | | ttaaTTAAccgagaaaaaaa | |
| VIR717 | SynRV | I-IEDG- | AACATCCCTCAAAagactca | 4038 |
| | tRNA- | L-Hek2 | aggaaaggctccagtggcgc | |
| | Ile- | | aatcggttagcgcgcggtac | |
| | Leu IEDG | | ttataagacagtgcacctgt | |
| | Hek2 ΔG | | gagcaatgccgaggttgtga | |
| | Abe820m- | | gttcaagcctcacctggagc | |
| | T2a- | | aGcgtGtAgggTaaccatga | |
| | mScarlet | | acGTTTTAGAGCTAGAAATA | |
| | | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAggtag | |
| | | | cgtggccgagcggtctaagg | |
| | | | cgctggattaaggctccagt | |
| | | | ctcttcgggggcgtgggttc | |
| | | | gaatcccaccgctgccaGGA | |
| | | | ACACAAAGCATAGACTGCgt | |
| | | | tttagagctaGCCgCGAGGA | |
| | | | AGGAGGTCTGAGGAGGTCAC | |
| | | | TGcGGCtagcaagttaaaat | |
| | | | aaggctagtccgttatcaac | |
| | | | ttgaaaaagtggcaccgagt | |
| | | | cggtgctttttaaTTAAccg | |
| | | | agaaaaaaa | |
| VIR718 | SynRV | I-IEDG- | AACATCCCTCAAAagactca | 4017 |
| | tRNA- | P-Hek2 | aggaaaggctccagtggcgc | |
| | Ile- | | aatcggttagcgcgcggtac | |
| | Pro IEDG | | ttataagacagtgcacctgt | |
| | Hek2 ΔG | | gagcaatgccgaggttgtga | |
| | Abe820m- | | gttcaagcctcacctggagc | |
| | T2a- | | aGcgtGtAgggTaaccatga | |
| | mScarlet | | acGTTTTAGAGCTAGAAATA | |
| | | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAggctc | |
| | | | gttggtctaggggtatgatt | |
| | | | ctcgcttagggtgcgagagg | |
| | | | tcccgggttcaaatcccgga | |
| | | | cgagcccGGAACACAAAGCA | |
| | | | TAGACTGCgttttagagcta | |
| | | | GCCgCGAGGAAGGAGGTCTG | |
| | | | AGGAGGTCACTGcGGCtagc | |
| | | | aagttaaaataaggctagtc | |
| | | | cgttatcaacttgaaaaagt | |
| | | | ggcaccgagtcggtgctttt | |
| | | | taaTTAA | |
| VIR719 | SynRV | I-IEDG- | AACATCCCTCAAAagactca | 4018 |
| | tRNA- | T-Hek2 | aggaaaggctccagtggcgc | |
| | Ile- | | aatcggttagcgcgcggtac | |
| | Thr | | ttataagacagtgcacctgt | |
| | IEDG | | gagcaatgccgaggttgtga | |
| | Hek2 ΔG | | gttcaagcctcacctggagc | |
| | Abe820m- | | aGcgtGtAgggTaaccatga | |
| | T2a- | | acGTTTTAGAGCTAGAAATA | |
| | mScarlet | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAggctc | |
| | | | catagctcaggggttagagc | |
| | | | actggtcttgtaaaccaggg | |
| | | | gtcgcgagttcaattctcgc | |
| | | | tggggcttGGAACACAAAGC | |
| | | | ATAGACTGCgttttagagct | |
| | | | aGCCgCGAGGAAGGAGGTCT | |
| | | | GAGGAGGTCACTGcGGCtag | |
| | | | caagttaaaataaggctagt | |
| | | | ccgttatcaacttgaaaaag | |
| | | | tggcaccgagtcggtgcttt | |
| | | | ttaaTTAAccgagaaaaaaa | |
| VIR720 | SynRV | I-IEDG- | AACATCCCTCAAAagactca | 4019 |
| | tRNA- | V-Hek2 | aggaaaggctccagtggcgc | |
| | Ile-Val | | aatcggttagcgcgcggtac | |
| | IEDG | | ttataagacagtgcacctgt | |
| | Hek2 | | gagcaatgccgaggttgtga | |
| | ΔG | | gttcaagcctcacctggagc | |
| | Abe820m- | | aGcgtGtAgggTaaccatga | |
| | T2a- | | acGTTTTAGAGCTAGAAATA | |
| | mScarlet | | GCAAGTTAAAATAAGGCTAG | |
| | | | TCCGTTATCAACTTGAAAAA | |
| | | | GTGGCACCGAGTCGGTGCTT | |
| | | | TTTTCACACACACAAgtttc | |
| | | | cgtagtgtagtggttatcac | |
| | | | gttcgcctcacacgcgaaag | |
| | | | gtccccggttcgaaaccggg | |
| | | | cggaaacaGGAACACAAAGC | |
| | | | ATAGACTGCgttttagagct | |
| | | | aGCCgCGAGGAAGGAGGTCT | |
| | | | GAGGAGGTCACTGcGGCtag | |
| | | | caagttaaaataaggctagt | |
| | | | ccgttatcaacttgaaaaag | |
| | | | tggcaccgaglcggtgcttt | |
| | | | ttaaTTAAccgagaaaaaaa | |

DNA538 Sequence:

| | | | | |
|---|---|---|---|---|
| DNA538 | EFS- | tRNA-Pro- | ggctcgttggtctaggggtat | (SEQ |
| | tRNA- | HEK2 | gattctcgcttagggtgcga | ID |
| | Pro- | gRNA | gaggtcccgggttcaaatcc | NO: |
| | HEK2 | | cggacgagcccGAACACAAA | 4020) |
| | gRNA | | GCATAGACTGCgtCttagag | |
| | | | ctaGGCCCGAGGAAGGAGGT | |
| | | | CTGAGGAGGTCACTGGGCCt | |
| | | | agcaagttaaGataaggcta | |
| | | | gtccgttatcaacttgaaaa | |
| | | | agtggcaccgagtcggtgct | |
| | | | taaccagtttgtgtcggctc | |
| | | | gttggtctaggggtatgatt | |
| | | | ctcgcttagggtgcgagagg | |
| | | | tcccgggttcaaatcccgga | |
| | | | cgagccc | |

VIR622 Sequence:

| | | | | |
|---|---|---|---|---|
| VIR622 | VIR622 | ABE8-20- | ATGtccgaagtcgagttttcc | (SEQ |
| | SynV | 2a-GFP | catgagtactggatgagaca | ID |
| | deIG | tRNA Ile- | cgcattgactctcgcaaaga | NO: |
| | ABE8-20- | Hek2 | gggctcgagatgaacgcgag | 4021) |
| | 2a-GFP | gRNA | gtgcccgtgggggcagtact | |
| | tRNA | | cgtgctcaacaatcgcgtaa | |
| | Ile- | | tcggcgaaggttggaatagg | |
| | Hek2 | | gcaatcggactccacgaccc | |
| | gRNA | | cactgcacatgcggaaatca | |
| | | | tggcccttcgacagggaggg | |
| | | | cttgtgatgcagaattatcg | |
| | | | acttatcgatgcgacgctgt | |
| | | | acgtcacgtttgaaccttgc | |
| | | | gtaatgtgcgcgggagctat | |
| | | | gattcactcccgcattggac | |
| | | | gagttgtattcggtgttcgc | |
| | | | aacgccaagacgggtgccgc | |
| | | | aggttcactgatggacgtgc | |
| | | | tgcattacccaggcatgaac | |
| | | | caccgggtagaaatcacaga | |
| | | | aggcatattggcggacgaat | |
| | | | gtgcggcgctgttgtgttac | |
| | | | ttttttcgcatgcccaggcg | |
| | | | tgtctttaacgcccagaaaa | |
| | | | aagcacaatcctctactgac | |
| | | | tctggtggttcttctggtgg | |
| | | | ttctagcggcagcgagactc | |
| | | | ccgggacctcagagtccgcc | |
| | | | acacccgaaagttctggtgg | |
| | | | ttcttctggtggttctgaca | |
| | | | agaagtacagcatcggcctg | |
| | | | gccatcggcaccaactctgt | |
| | | | gggctgggccgtgatcaccg | |
| | | | acgagtacaaggtgcccagc | |
| | | | aagaaattcaaggtgctggg | |
| | | | caacaccgaccggcacagca | |
| | | | tcaagaagaacctgatcgga | |
| | | | gccctgctgttcgacagcgg | |
| | | | cgaaacagccgaggccaccc | |
| | | | ggctgaagagaaccgccaga | |
| | | | agaagatacaccagacggaa | |
| | | | gaaccggatctgctatctgc | |
| | | | aagagatcttcagcaacgag | |
| | | | atggccaaggtggacgacag | |
| | | | cttcttccacagactggaag | |
| | | | agtccttcctggtggaagag | |
| | | | gataagaagcacgagcggca | |
| | | | ccccatcttcggcaacatcg | |
| | | | tggacgaggtggcctaccac | |
| | | | gagaagtaccccaccatcta | |
| | | | ccacctgagaaagaaactgg | |
| | | | tggacagcaccgacaaggcc | |
| | | | gacctgcggctgatctatct | |
| | | | ggccctggcccacatgatca | |
| | | | agttccggggccacttcctg | |
| | | | atcgagggcgacctgaaccc | |
| | | | cgacaacagcgacgtggaca | |
| | | | agctgttcatccagctggtg | |
| | | | cagacctacaaccagctgtt | |
| | | | cgaggaaaaccccatcaacg | |
| | | | ccagcggcgtggacgccaag | |
| | | | gccatcctgtctgccagact | |
| | | | gagcaagagcagacggctgg | |
| | | | aaaatctgatcgcccagctg | |
| | | | cccggcgagaagaagaatgg | |
| | | | cctgttcggaaacctgattg | |
| | | | ccctgagcctgggcctgacc | |
| | | | cccaacttcaagagcaactt | |
| | | | cgacctggccgaggatgcca | |
| | | | aactgcagctgagcaaggac | |
| | | | acctacgacgacgacctgga | |
| | | | caacctgctggcccagatcg | |
| | | | gcgaccagtacgccgacctg | |
| | | | tttctggccgccaagaacct | |
| | | | gtccgacgccatcctgctga | |
| | | | gcgacatcctgagagtgaac | |
| | | | accgagatcaccaaggcccc | |
| | | | cctgagcgcctctatgatca | |
| | | | agagatacgacgagcaccac | |
| | | | caggacctgaccctgctgaa | |
| | | | agctctcgtgcggcagcagc | |
| | | | tgcctgagaagtacaaagag | |
| | | | attttcttcgaccagagcaa | |
| | | | gaacggctacgccggctaca | |
| | | | ttgacggcggagccagccag | |
| | | | gaagagttctacaagttcat | |
| | | | caagcccatcctggaaaaga | |
| | | | tggacggcaccgaggaactg | |
| | | | ctcgtgaagctgaacagaga | |
| | | | ggacctgctgcggaagcagc | |
| | | | ggaccttcgacaacggcagc | |
| | | | atcccccaccagatccacct | |
| | | | gggagagctgcacgccattc | |
| | | | tgcggcggcaggaagatttt | |
| | | | tacccattcctgaaggacaa | |
| | | | ccgggaaaagatcgagaaga | |
| | | | tcctgaccttccgcatcccc | |
| | | | tactacgtgggccctctggc | |
| | | | caggggaaacagcagattcg | |
| | | | cctggatgaccagaaagagc | |
| | | | gaggaaaccatcaccccctg | |
| | | | gaacttcgaggaagtggtgg | |
| | | | acaagggcgcttccgcccag | |
| | | | agcttcatcgagcggatgac | |
| | | | caacttcgataagaacctgc | |
| | | | ccaacgagaaggtgctgccc | |
| | | | aagcacagcctgctgtacga | |
| | | | gtacttcaccgtgtataacg | |
| | | | agctgaccaaagtgaaatac | |
| | | | gtgaccgagggaatgagaaa | |
| | | | gcccgccttcctgagcggcg | |
| | | | agcagaaaaaggccatcgtg | |
| | | | gacctgctgttcaagaccaa | |
| | | | ccggaaagtgaccgtgaagc | |
| | | | agctgaaagaggactacttc | |
| | | | aagaaaatcgagtgcttcga | |
| | | | ctccgtggaaatctccggcg | |
| | | | tggaagatcggttcaacgcc | |
| | | | tccctgggcacataccacga | |
| | | | tctgctgaaaattatcaagg | |
| | | | acaaggacttcctggacaat | |
| | | | gaggaaaacgaggacattct | |
| | | | ggaagatatcgtgctgaccc | |
| | | | tgacactgtttgaggacaga | |
| | | | gagatgatcgaggaacggct | |
| | | | gaaaacctatgcccacctgt | |
| | | | tcgacgacaaagtgatgaag | |
| | | | cagctgaagcggcggagata | |
| | | | caccggctggggcaggctga | |
| | | | gccggaagctgatcaacggc | |
| | | | atccgggacaagcagtccgg | |
| | | | caagacaatcctggatttcc | |
| | | | tgaagtccgacggcttcgcc | |
| | | | aacagaaacttcatgcagct | |
| | | | gatccacgacgacagcctga | |
| | | | cctttaaagaggacatccag | |
| | | | aaagcccaggtgtccggcca | |
| | | | gggcgatagcctgcacgagc | |
| | | | acattgccaatctggccggc | |
| | | | agccccgccattaagaaggg | |
| | | | catcctgcagacagtgaagg | |
| | | | tggtggacgagctcgtgaaa | |
| | | | gtgatgggccggcacaagcc | |
| | | | cgagaacatcgtgatcgaaa | |
| | | | tggccagagagaaccagacc | |
| | | | acccagaagggacagaagaa | |
| | | | cagccgcgagagaatgaagc | |
| | | | ggatcgaagagggcatcaaa | |
| | | | gagctgggcagccagatcct | |
| | | | gaaagaacaccccgtggaaa | |
| | | | acacccagctgcagaacgag | |
| | | | aagctgtacctgtactacct | |
| | | | gcagaatgggcgggatatgt | |
| | | | acgtggaccaggaactggac | |
| | | | atcaaccggctgtccgacta | |
| | | | cgatgtggaccatatcgtgc | |
| | | | ctcagagctttctgaaggac | |
| | | | gactccatcgacaacaaggt | |
| | | | gctgaccagaagcgacaaga | |
| | | | accggggcaagagcgacaac | |
| | | | gtgccctccgaagaggtcgt | |
| | | | gaagaagatgaagaactact | |
| | | | ggcggcagctgctgaacgcc | |
| | | | aagctgattacccagagaaa | |
| | | | gttcgacaatctgaccaagg | |
| | | | ccgagagaggcggcctgagc | |
| | | | gaactggataaggccggctt | |
| | | | catcaagagacagctggtgg | |
| | | | aaacccggcagatcacaaag | |
| | | | cacgtggcacagatcctgga | |
| | | | ctcccggatgaacactaagt | |
| | | | acgacgagaatgacaagctg | |
| | | | atccgggaagtgaaagtgat | |
| | | | caccctgaagtccaagctgg | |
| | | | tgtccgatttccggaaggat | |
| | | | ttccagttttacaaagtgcg | |
| | | | cgagatcaacaactaccacc | |
| | | | acgcccacgacgcctacctg | |
| | | | aacgccgtcgtgggaaccgc | |
| | | | cctgatcaaaaagtacccta | |
| | | | agctggaaagcgagttcgtg | |
| | | | tacggcgactacaaggtgta | |
| | | | cgacgtgcggaagatgatcg | |
| | | | ccaagagcgagcaggaaatc | |
| | | | ggcaaggctaccgccaagta | |
| | | | cttcttctacagcaacatca | |
| | | | tgaactttttcaagaccgag | |
| | | | attaccctggccaacggcga | |
| | | | gatccggaagcggcctctga | |
| | | | tcgagacaaacggcgaaacc | |
| | | | ggggagatcgtgtgggataa | |
| | | | gggccgggattttgccaccg | |
| | | | tgcggaaagtgctgagcatg | |
| | | | ccccaagtgaatatcgtgaa | |
| | | | aaagaccgaggtgcagacag | |
| | | | gcggcttcagcaaagagtct | |
| | | | atcctgcccaagaggaacag | |
| | | | cgataagctgatcgccagaa | |
| | | | agaaggactgggaccctaag | |
| | | | aagtacggcggcttcgacag | |
| | | | ccccaccgtggcctattctg | |
| | | | tgctggtggtggccaaagtg | |
| | | | gaaaagggcaagtccaagaa | |
| | | | actgaagagtgtgaaagagc | |
| | | | tgctggggatcaccatcatg | |
| | | | gaaagaagcagcttcgagaa | |
| | | | gaatcccatcgactttctgg | |
| | | | aagccaagggctacaaagaa | |
| | | | gtgaaaaaggacctgatcat | |
| | | | caagctgcctaagtactccc | |
| | | | tgttcgagctggaaaacggc | |
| | | | cggaagagaatgctggcctc | |
| | | | tgccggcgaactgcagaagg | |
| | | | gaaacgaactggccctgccc | |
| | | | tccaaatatgtgaacttcct | |
| | | | gtacctggccagccactatg | |
| | | | agaagctgaagggctccccc | |
| | | | gaggataatgagcagaaaca | |
| | | | gctgtttgtggaacagcaca | |
| | | | agcactacctggacgagatc | |
| | | | atcgagcagatcagcgagtt | |
| | | | ctccaagagagtgatcctgg | |
| | | | ccgacgctaatctggacaaa | |
| | | | gtgctgtccgcctacaacaa | |
| | | | gcaccgggataagcccatca | |
| | | | gagagcaggccgagaatatc | |
| | | | atccacctgtttaccctgac | |
| | | | caatctgggagcccctgccg | |
| | | | ccttcaagtactttgacacc | |
| | | | accatcgaccggaagaggta | |
| | | | caccagcaccaaagaggtgc | |
| | | | tggacgccaccctgatccac | |
| | | | cagagcatcaccggcctgta | |
| | | | cgagacacggatcgacctgt | |
| | | | ctcagctgggaggtgacgag | |
| | | | ggagctgataagcgcaccgc | |
| | | | cgatggttccgagttcgaaa | |
| | | | gccccaagaagaagaggaaa | |
| | | | gtcGAATTCGGCAGTGGAGA | |
| | | | GGGCAGAGGgtccCTGCTAA | |
| | | | CATGCGGTGACGTCGAGGAG | |
| | | | AATCCtGGcCCaatggtgag | |
| | | | caagggcgaggagctgttca | |
| | | | ccggggtggtgcccatcctg | |
| | | | gtcgagctggacggcgacgt | |
| | | | aaacggccacaagttcagcg | |
| | | | tgtccggcgagggcgagggc | |
| | | | gatgccacctacggcaagct | |
| | | | gaccctgaagttcatctgca | |
| | | | ccaccggcaagctgcccgtg | |
| | | | ccctggcccaccctcgtgac | |
| | | | caccctgacctacggcgtgc | |
| | | | agtgcttcagccgctacccc | |
| | | | gaccacatgaagcagcacga | |
| | | | cttcttcaagtccgccatgc | |
| | | | ccgaaggctacgtccaggag | |
| | | | cgcaccatcttcttcaagga | |
| | | | cgacggcaactacaagaccc | |
| | | | gcgccgaggtgaagttcgag | |
| | | | ggcgacaccctggtgaaccg | |
| | | | catcgagctgaagggcatcg | |
| | | | acttcaaggaggacggcaac | |
| | | | atcctggggcacaagctgga | |
| | | | gtacaactacaacagccaca | |
| | | | acgtctatatcatggccgac | |
| | | | aagcagaagaacggcatcaa | |
| | | | ggtgaacttcaagatccgcc | |
| | | | acaacatcgaggacggcagc | |
| | | | gtgcagctcgccgaccacta | |
| | | | ccagcagaacacccccatcg | |
| | | | gcgacggccccgtgctgctg | |
| | | | cccgacaaccactacctgag | |
| | | | cacccagtccgccctgagca | |
| | | | aagaccccaacgagaagcgc | |
| | | | gatcacatggtcctgctgga | |
| | | | gttcgtgaccgccgccggga | |
| | | | tcactctcggcatggacgag | |
| | | | ctgtacaagTAAGAAGTTGA | |
| | | | ATAACAAAATGCCGGAAATC | |
| | | | TACGGATTGTGTATATCCAT | |
| | | | CATGAAAAAAACTAACACCC | |
| | | | CTCCTTTCGAACCATCCCAA | |
| | | | ACgctccagtggcgcaatcg | |
| | | | gttagcgcgcggtacttata | |
| | | | agacagtgcacctgtgagca | |
| | | | atgccgaggttgtgagttca | |
| | | | agcctcacctggagcaGGAA | |
| | | | CACAAAGCATAGACTGCgtt | |
| | | | ttagagctaGAAAtagcaag | |
| | | | ttaaaataaggctagtccgt | |
| | | | tatcaacttgaaaaagtggc | |
| | | | accgagtcggtgcttCACAC | |
| | | | ACACAAgctccagtggcgca | |
| | | | atcggttagcgcgcggtact | |
| | | | tataagacagtgcaGCCgCG | |
| | | | AGGAAGGAGGTCTGAGGAGG | |
| | | | TCACTGcGGCcctgtgagca | |
| | | | atgccgaggttgtgagttca | |
| | | | agcctcacctggagcata | |

tRNA-qRNA-tRNA cassette (in VIR622):
gctccagtggcgcaatcggttagcgcgcggtacttataaga
cagtgcacctgtgagcaatgccgaggttgtgagttcaagc
ctcacctggagcaGGAACACAAAGCATAGACTGCgtttta
gagctaGAAAtagcaagttaaaataaggctagtccgttat
caacttgaaaaagtggcaccgagtcggtgcttCACACACA
CAAgctccagtggcgcaatcggttagcgcgcggtacttat
aagacagtgcaGCCgCGAGGAAGGAGGTCTGAGGAGGTCA
CTGcGGCcctgtgagcaatgccgaggttgtgagttcaagc
ctcacctggagca (SEQ ID NO: 4022)

### Example 6: Recombinant Rabies Virus Particle Gene Delivery—Additional Transgenes

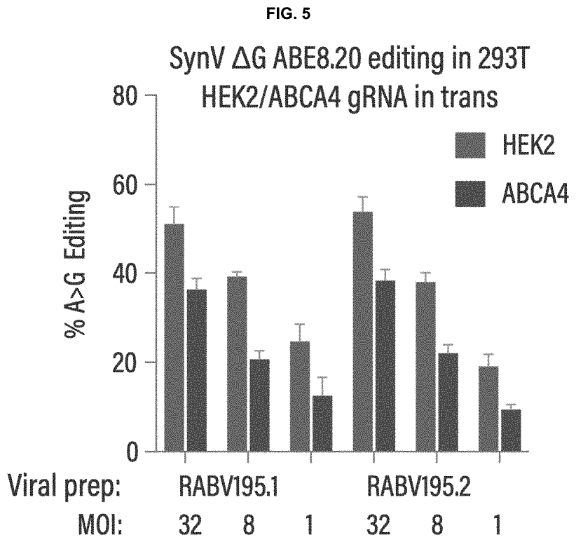

A codon-optimized version of the SAD B19 ΔG RABV genome, referred to as SynV, was used to encode the base editor ABE8.20, where the therapeutic cargo (e.g., the base editor) replaced the G protein within the viral genome. U6-driven gRNAs, targeting HEK2 and ABCA4 loci, were transfected in trans into target 293T cells at the time of infection. Multiplicity of infection (MOI) of 32, 8, and 1, based on functional viral titers, were used. As shown in FIG. 5, A to G base editing was observed in 293T cells tranduced with RABV having a RABV genome encoding the ABE 8.20 base editor.

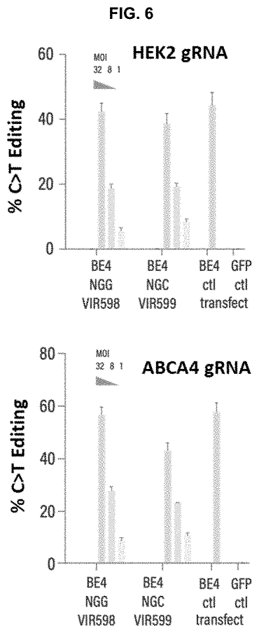

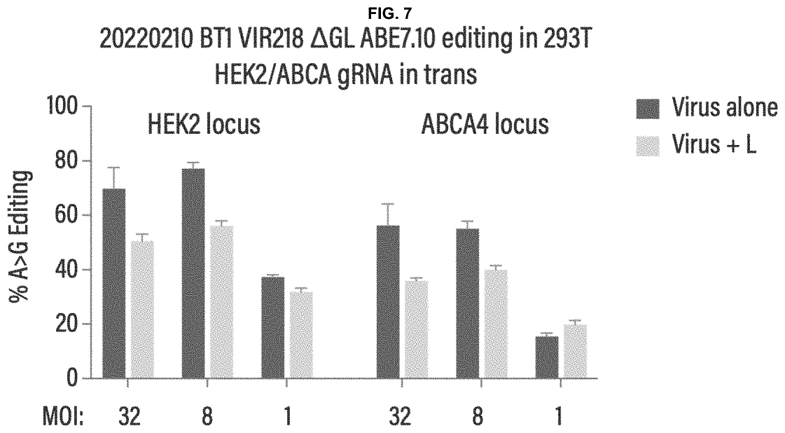

The SynV ΔG RABV genome was used to deliver several other base editors into 293T cells. As shown in FIG. 6, C to T base editing was observed in 293T cells tranduced with RABV having a RABV genome encoding one of several BE4 base editors.

In addition to the above described ΔG RABV genome, a ΔGL RABV genome was used to deliver the base editor ABE7.10. As shown in FIG. 7, A to G base editing was observed in 293T cells tranduced with RABV having a RABV genome encoding ABE7.10. One set of cells was additionally transfected by a plasmid encoding RABV L protein (virus+L).

In each of the above experiments represented by FIG. 5 to FIG. 7, robust base editing was observed in cells transduced with RABV expressing one of several different base editors. This robust activity was observed in both a ΔG and ΔGL RABV genome, at numerous MOIs, and at two distinct sites in the cell geneome (the HEK2 and ABCA4 loci).

### Example 7: Recombinant Rabies Virus Genome—Additional Genome Architectures

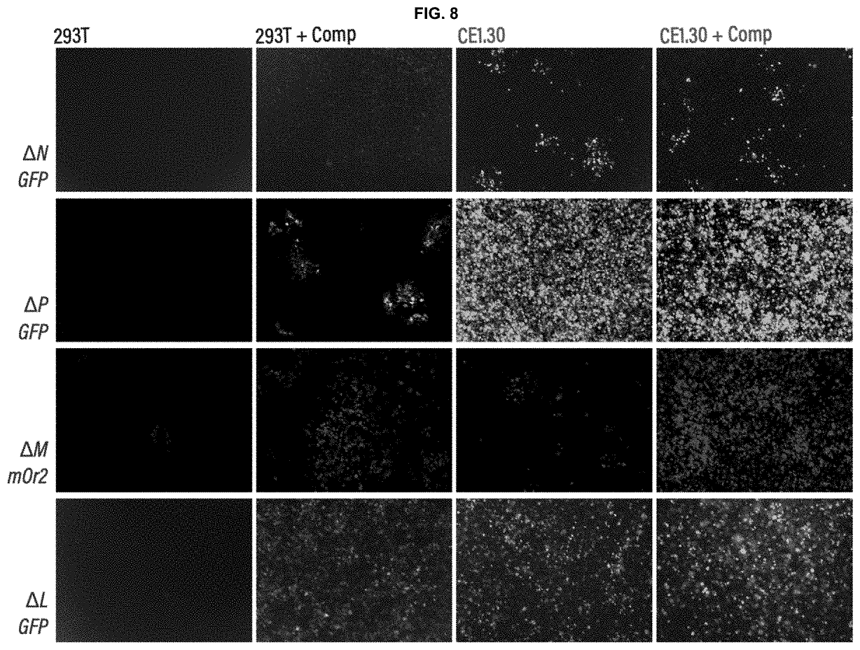

Example 4 described the use of a recombinant rabies virus genome in a ΔALL (i.e., ΔGLNPM) background. Additional RABV genome architectures were next tested to determine if transgene expression was possible. 293T cells and the cell line CE1.30 described above were used for viral production. The cells were transfected with rabies virus vectors in a ΔN, ΔP, ΔM, and ΔL background, each vector encoding a Cre recombinase-2A-GFP reporter gene. The 293T cells or CE1.30 cells were transfected with or without complementation of the missing gene (e.g., the ΔN vector was co-transfected with an N gene expressing vector, the ΔP vector was co-transfected with a P gene expressing vector, etc.). Flourescent images were taken 6 days post-transfection. As shown in FIG. 8, the CE1.30 cell line supported the growth of the ΔN, ΔP, ΔM, and ΔL replicons, with or without the additional complementation of the missing gene.

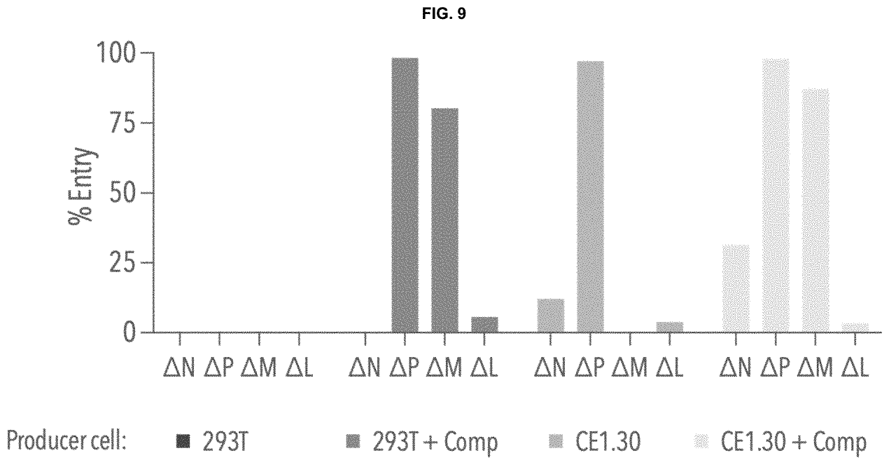

The cell supernatant of the above recited cell transfections was next applied to untranfected 293T cells to determine % viral entry of the rabies viruses. The % viral entry was measured by GFP flourescence. As shown in FIG. 9, % viral entry into cells was detected for each rabies virus vector tested in the CE1.30 cell line.

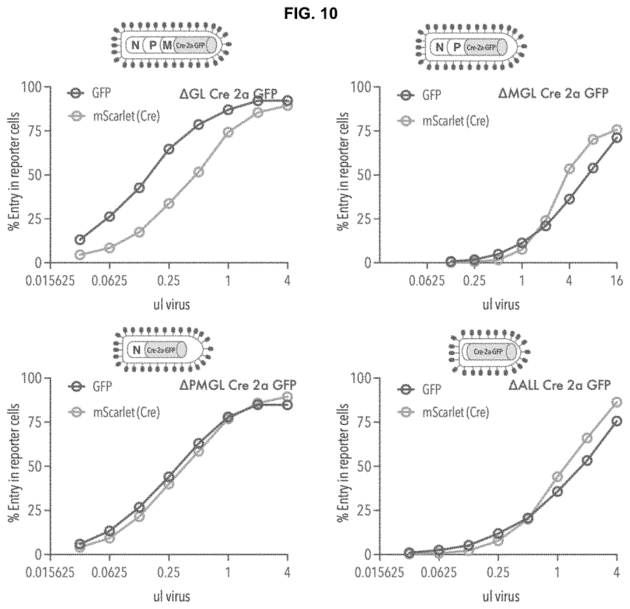

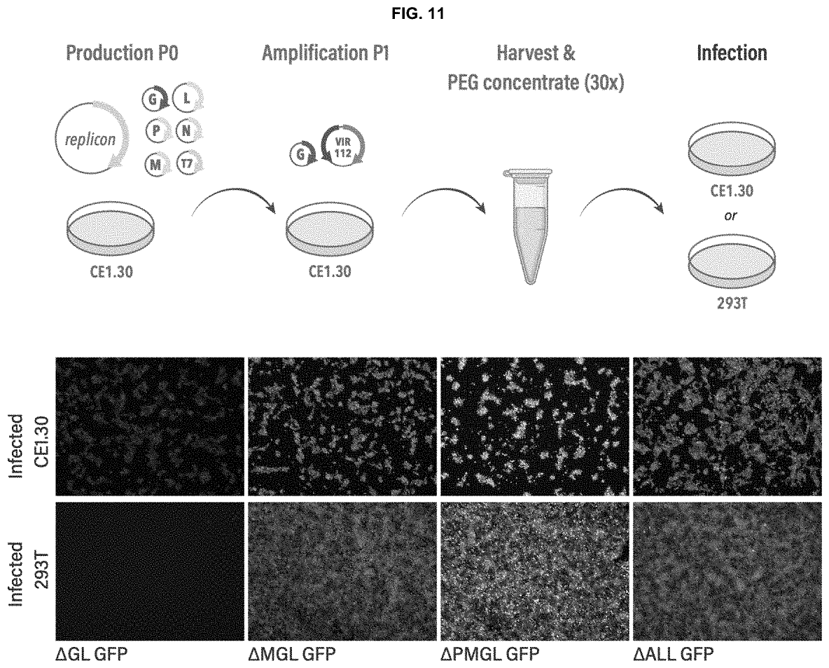

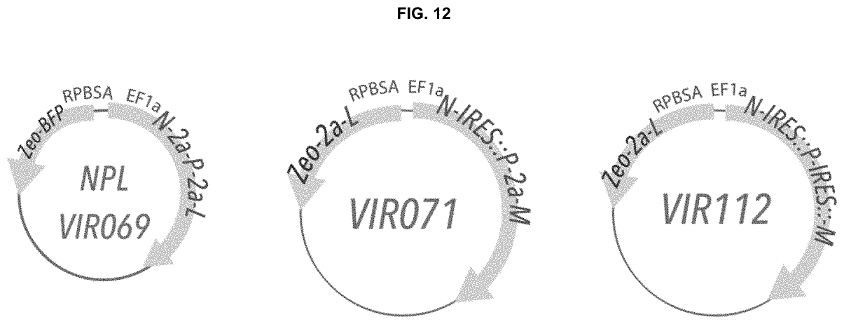

Further RABV genome architectures of ΔMGL and ΔPMGL were tested in a similar manner with viral production being done in the CE1.30 cell line. ΔGL, ΔMGL, ΔPMGL, and ΔALL vectors encoding Cre-2a-GFP as transgene were grown in CE1.30 cells, and titered on reporter cells to show functional delivery of both Cre and GFP mRNA. As shown in FIG. 10, viral entry was detetected in all RABV genome backgrounds, as measured by GFP and mScarlet flourescence. GFP transgene expression was also detected by flourescence, as shown in FIG. 11.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### L. SEQUENCE LISTING

| | SEQ | |
|---|---|---|
| Descrip- | ID | |
| tion | NO: | Sequence |
| Adenosine | 8 | MSEVEFSHEYWMRHALTLAKRARDEREVPV |
| Deaminase | | GAVLVLNNRVIGEGWNRAIGLHDPTAHAEI |
| Reference | | MALRQGGLVMQNYRLIDATLYVTFEPCVMC |
| Sequence | | AGAMIHSRIGRVVFGVRNAKTGAAGSLMDV |
| | | LHYPGMNHRVEITEGILADECAALLCYFFR |
| | | MPRQVFNAQKKAQSSTD |
| BhCas12b | 274 | GCCACCATGGCCCCAAAGAAGAAGCGGAAG |
| GGSGGS- | | GTCGGTATCCACGGAGTCCCAGCAGCCGCC |
| ABE8- | | ACCAGATCCTTCATCCTGAAGATCGAGCCC |
| Xten20 | | AACGAGGAAGTGAAGAAAGGCCTCTGGAAA |
| at P153 | | ACCCACGAGGTGCTGAACCACGGAATCGCC |
| poly- | | TACTACATGAATATCCTGAAGCTGATCCGG |
| nucleotide | | CAAGAGGCCATCTACGAGCACCACGAGCAG |
| | | GACCCCAAGAATCCCAAGAAGGTGTCCAAG |
| | | GCCGAGATCCAGGCCGAGCTGTGGGATTTC |
| | | GTGCTGAAGATGCAGAAGTGCAACAGCTTC |
| | | ACACACGAGGTGGACAAGGACGAGGTGTTC |
| | | AACATCCTGAGAGAGCTGTACGAGGAACTG |
| | | GTGCCCAGCAGCGTGGAAAAGAAGGGCGAA |
| | | GCCAACCAGCTGAGCAACAAGTTTCTGTAC |
| | | CCTCTGGTGGACCCCAACAGCCAGTCTGGA |
| | | AAGGGAACAGCCAGCAGCGGCAGAAAGCCC |
| | | AGATGGTACAACCTGAAGATTGCCGGCGAT |
| | | CCCGGAGGCTCTGGAGGAAGCTCCGAAGTC |
| | | GAGTTTTCCCATGAGTACTGGATGAGACAC |
| | | GCATTGACTCTCGCAAAGAGGGCTCGAGAT |
| | | GAACGCGAGGTGCCCGTGGGGGCAGTACTC |
| | | GTGCTCAACAATCGCGTAATCGGCGAAGGT |
| | | TGGAATAGGGCAATCGGACTCCACGACCCC |
| | | ACTGCACATGCGGAAATCATGGCCCTTCGA |
| | | CAGGGAGGGCTTGTGATGCAGAATTATCGA |
| | | CTTTATGATGCGACGCTGTACGTCACGTTT |
| | | GAACCTTGCGTAATGTGCGCGGGAGCTATG |
| | | ATTCACTCCCGCATTGGACGAGTTGTATTC |
| | | GGTGTTCGCAACGCCAAGACGGGTGCCGCA |
| | | GGTTCACTGATGGACGTGCTGCATCATCCA |
| | | GGCATGAACCACCGGGTAGAAATCACAGAA |
| | | GGCATATTGGCGGACGAATGTGCGGCGCTG |
| | | TTGTGTCGTTTTTTTCGCATGCCCAGGCGG |
| | | GTCTTTAACGCCCAGAAAAAAGCACAATCC |
| | | TCTACTGACGGCTCTTCTGGATCTGAAACA |
| | | CCTGGCACAAGCGAGAGCGCCACCCCTGAG |
| | | AGCTCTGGCTCCTGGGAAGAAGAGAAGAAG |
| | | AAGTGGGAAGAAGATAAGAAAAAGGACCCG |
| | | CTGGCCAAGATCCTGGGCAAGCTGGCTGAG |
| | | TACGGACTGATCCCTCTGTTCATCCCCTAC |
| | | ACCGACAGCAACGAGCCCATCGTGAAAGAA |
| | | ATCAAGTGGATGGAAAAGTCCCGGAACCAG |
| | | AGCGTGCGGCGGCTGGATAAGGACATGTTC |
| | | ATTCAGGCCCTGGAACGGTTCCTGAGCTGG |
| | | GAGAGCTGGAACCTGAAAGTGAAAGAGGAA |
| | | TACGAGAAGGTCGAGAAAGAGTACAAGACC |
| | | CTGGAAGAGAGGATCAAAGAGGACATCCAG |
| | | GCTCTGAAGGCTCTGGAACAGTATGAGAAA |
| | | GAGCGGCAAGAACAGCTGCTGCGGGACACC |
| | | CTGAACACCAACGAGTACCGGCTGAGCAAG |
| | | AGAGGCCTTAGAGGCTGGCGGGAAATCATC |
| | | CAGAAATGGCTGAAAATGGACGAGAACGAG |
| | | CCCTCCGAGAAGTACCTGGAAGTGTTCAAG |
| | | GACTACCAGCGGAAGCACCCTAGAGAGGCC |
| | | GGCGATTACAGCGTGTACGAGTTCCTGTCC |
| | | AAGAAAGAGAACCACTTCATCTGGCGGAAT |
| | | CACCCTGAGTACCCCTACCTGTACGCCACC |
| | | TTCTGCGAGATCGACAAGAAAAAGAAGGAC |
| | | GCCAAGCAGCAGGCCACCTTCACACTGGCC |
| | | GATCCTATCAATCACCCTCTGTGGGTCCGA |
| | | TTCGAGGAAAGAAGCGGCAGCAACCTGAAC |
| | | AAGTACAGAATCCTGACCGAGCAGCTGCAC |
| | | ACCGAGAAGCTGAAGAAAAAGCTGACAGTG |
| | | CAGCTGGACCGGCTGATCTACCCTACAGAA |
| | | TCTGGCGGCTGGGAAGAGAAGGGCAAAGTG |
| | | GACATTGTGCTGCTGCCCAGCCGGCAGTTC |
| | | TACAACCAGATCTTCCTGGACATCGAGGAA |
| | | AAGGGCAAGCACGCCTTCACCTACAAGGAT |
| | | GAGAGCATCAAGTTCCCTCTGAAGGGCACA |
| | | CTCGGCGGAGCCAGAGTGCAGTTCGACAGA |
| | | GATCACCTGAGAAGATACCCTCACAAGGTG |
| | | GAAAGCGGCAACGTGGGCAGAATCTACTTC |
| | | AACATGACCGTGAACATCGAGCCTACAGAG |
| | | TCCCCAGTGTCCAAGTCTCTGAAGATCCAC |
| | | CGGGACGACTTCCCCAAGGTGGTCAACTTC |
| | | AAGCCCAAAGAACTGACCGAGTGGATCAAG |
| | | GACAGCAAGGGCAAGAAACTGAAGTCCGGC |
| | | ATCGAGTCCCTGGAAATCGGCCTGAGAGTG |
| | | ATGAGCATCGACCTGGGACAGAGACAGGCC |
| | | GCTGCCGCCTCTATTTTCGAGGTGGTGGAT |
| | | CAGAAGCCCGACATCGAAGGCAAGCTGTTT |
| | | TTCCCAATCAAGGGCACCGAGCTGTATGCC |
| | | GTGCACAGAGCCAGCTTCAACATCAAGCTG |
| | | CCCGGCGAGACACTGGTCAAGAGCAGAGAA |
| | | GTGCTGCGGAAGGCCAGAGAGGACAATCTG |
| | | AAACTGATGAACCAGAAGCTCAACTTCCTG |
| | | CGGAACGTGCTGCACTTCCAGCAGTTCGAG |
| | | GACATCACCGAGAGAGAGAAGCGGGTCACC |
| | | AAGTGGATCAGCAGACAAGAGAACAGCGAC |
| | | GTGCCCCTGGTGTACCAGGATGAGCTGATC |
| | | CAGATCCGCGAGCTGATGTACAAGCCTTAC |
| | | AAGGACTGGGTCGCCTTCCTGAAGCAGCTC |
| | | CACAAGAGACTGGAAGTCGAGATCGGCAAA |
| | | GAAGTGAAGCACTGGCGGAAGTCCCTGAGC |
| | | GACGGAAGAAAGGGCCTGTACGGCATCTCC |
| | | CTGAAGAACATCGACGAGATCGATCGGACC |
| | | CGGAAGTTCCTGCTGAGATGGTCCCTGAGG |
| | | CCTACCGAACCTGGCGAAGTGCGTAGACTG |
| | | GAACCCGGCCAGAGATTCGCCATCGACCAG |
| | | CTGAATCACCTGAACGCCCTGAAAGAAGAT |
| | | CGGCTGAAGAAGATGGCCAACACCATCATC |
| | | ATGCACGCCCTGGGCTACTGCTACGACGTG |
| | | CGGAAGAAGAAATGGCAGGCTAAGAACCCC |
| | | GCCTGCCAGATCATCCTGTTCGAGGATCTG |
| | | AGCAACTACAACCCCTACGAGGAAAGGTCC |
| | | CGCTTCGAGAACAGCAAGCTCATGAAGTGG |
| | | TCCAGACGCGAGATCCCCAGACAGGTTGCA |
| | | CTGCAGGGCGAGATCTATGGCCTGCAAGTG |
| | | GGAGAAGTGGGCGCTCAGTTCAGCAGCAGA |
| | | TTCCACGCCAAGACAGGCAGCCCTGGCATC |
| | | AGATGTAGCGTCGTGACCAAAGAGAAGCTG |
| | | CAGGACAATCGGTTCTTCAAGAATCTGCAG |
| | | AGAGAGGGCAGACTGACCCTGGACAAAATC |
| | | GCCGTGCTGAAAGAGGGCGATCTGTACCCA |
| | | GACAAAGGCGGCGAGAAGTTCATCAGCCTG |
| | | AGCAAGGATCGGAAGTGCGTGACCACACAC |
| | | GCCGACATCAACGCCGCTCAGAACCTGCAG |
| | | AAGCGGTTCTGGACAAGAACCCACGGCTTC |
| | | TACAAGGTGTACTGCAAGGCCTACCAGGTG |
| | | GACGGCCAGACCGTGTACATCCCTGAGAGC |
| | | AAGGACCAGAAGCAGAAGATCATCGAAGAG |
| | | TTCGGCGAGGGCTACTTCATTCTGAAGGAC |
| | | GGGGTGTACGAATGGGTCAACGCCGGCAAG |
| | | CTGAAAATCAAGAAGGGCAGCTCCAAGCAG |
| | | AGCAGCAGCGAGCTGGTGGATAGCGACATC |
| | | CTGAAAGACAGCTTCGACCTGGCCTCCGAG |
| | | CTGAAAGGCGAAAAGCTGATGCTGTACAGG |
| | | GACCCCAGCGGCAATGTGTTCCCCAGCGAC |
| | | AAATGGATGGCCGCTGGCGTGTTCTTCGGA |
| | | AAGCTGGAACGCATCCTGATCAGCAAGCTG |
| | | ACCAACCAGTACTCCATCAGCACCATCGAG |
| | | GACGACAGCAGCAAGCAGTCTATGAAAAGG |
| | | CCGGCGGCCACGAAAAAGGCCGGCCAGGCA |
| | | AAAAAGAAAAAGGGATCCTACCCATACGAT |
| | | GTTCCAGATTACGCTTATCCCTACGACGTG |
| | | CCTGATTATGCATACCCATATGATGTCCCC |
| | | GACTATGCCTAA |
| BhCas12b | 275 | MAPKKKRKVGIHGVPAAATRSFILKIEPNE |
| GGSGGS- | | EVKKGLWKTHEVLNHGIAYYMNILKLIRQE |
| ABE8- | | AIYEHHEQDPKNPKKVSKAEIQAELWDFVL |
| Xten20 at | | KMQKCNSFTHEVDKDEVFNILRELYEELVP |
| P153 | | SSVEKKGEANQLSNKFLYPLVDPNSQSGKG |
| polypeptide | | TASSGRKPRWYNLKIAGDPGGSGGSSEVEF |
| | | SHEYWMRHALTLAKRARDEREVPVGAVLVL |
| | | NNRVIGEGWNRAIGLHDPTAHAEIMALRQG |
| | | GLVMQNYRLYDATLYVTFEPCVMCAGAMIH |
| | | SRIGRVVFGVRNAKTGAAGSLMDVLHHPGM |
| | | NHRVEITEGILADECAALLCRFFRMPRRVF |
| | | NAQKKAQSSTDGSSGSETPGTSESATPESS |
| | | GSWEEEKKKWEEDKKKDPLAKILGKLAEYG |
| | | LIPLFIPYTDSNEPIVKEIKWMEKSRNQSV |
| | | RRLDKDMFIQALERFLSWESWNLKVKEEYE |
| | | KVEKEYKTLEERIKEDIQALKALEQYEKER |
| | | QEQLLRDTLNTNEYRLSKRGLRGWREIIQK |
| | | WLKMDENEPSEKYLEVFKDYQRKHPREAGD |
| | | YSVYEFLSKKENHFIWRNHPEYPYLYATFC |
| | | EIDKKKKDAKQQATFTLADPINHPLWVRFE |
| | | ERSGSNLNKYRILTEQLHTEKLKKKLTVQL |
| | | DRLIYPTESGGWEEKGKVDIVLLPSRQFYN |
| | | QIFLDIEEKGKHAFTYKDESIKFPLKGTLG |
| | | GARVQFDRDHLRRYPHKVESGNVGRIYFNM |
| | | TVNIEPTESPVSKSLKIHRDDFPKWNFKPK |
| | | ELTEWIKDSKGKKLKSGIESLEIGLRVMSI |
| | | DLGQRQAAAASIFEVVDQKPDIEGKLFFPI |
| | | KGTELYAVHRASFNIKLPGETLVKSREVLR |
| | | KAREDNLKLMNQKLNFLRNVLHFQQFEDIT |
| | | EREKRVTKWISRQENSDVPLVYQDELIQIR |
| | | ELMYKPYKDWVAFLKQLHKRLEVEIGKEVK |
| | | HWRKSLSDGRKGLYGISLKNIDEIDRTRKF |
| | | LLRWSLRPTEPGEVRRLEPGQRFAIDQLNH |
| | | LNALKEDRLKKMANTIIMHALGYCYDVRKK |
| | | KWQAKNPACQIILFEDLSNYNPYEERSRFE |
| | | NSKLMKWSRREIPRQVALQGEIYGLQVGEV |
| | | GAQFSSRFHAKTGSPGIRCSWTKEKLQDNR |
| | | FFKNLQREGRLTLDKIAVLKEGDLYPDKGG |
| | | EKFISLSKDRKCVTTHADINAAQNLQKRFW |
| | | TRTHGFYKVYCKAYQVDGQTVYIPESKDQK |
| | | QKIIEEFGEGYFILKDGVYEWVNAGKLKIK |
| | | KGSSKQSSSELVDSDILKDSFDLASELKGE |
| | | KLMLYRDPSGNVFPSDKWMAAGVFFGKLER |
| | | ILISKLTNQYSISTIEDDSSKQSMKRPAAT |
| | | KKAGQAKKKKGSYPYDVPDYAYPYDVPDYA |
| | | YPYDVPDYA |
| BhCas12b | 276 | GCCACCATGGCCCCAAAGAAGAAGCGGAAG |
| GGSGGS- | | GTCGGTATCCACGGAGTCCCAGCAGCCGCC |
| ABE8- | | ACCAGATCCTTCATCCTGAAGATCGAGCCC |
| Xten20 | | AACGAGGAAGTGAAGAAAGGCCTCTGGAAA |
| at K255 | | ACCCACGAGGTGCTGAACCACGGAATCGCC |
| poly- | | TACTACATGAATATCCTGAAGCTGATCCGG |
| nucleotide | | CAAGAGGCCATCTACGAGCACCACGAGCAG |
| | | GACCCCAAGAATCCCAAGAAGGTGTCCAAG |
| | | GCCGAGATCCAGGCCGAGCTGTGGGATTTC |
| | | GTGCTGAAGATGCAGAAGTGCAACAGCTTC |
| | | ACACACGAGGTGGACAAGGACGAGGTGTTC |
| | | AACATCCTGAGAGAGCTGTACGAGGAACTG |
| | | GTGCCCAGCAGCGTGGAAAAGAAGGGCGAA |
| | | GCCAACCAGCTGAGCAACAAGTTTCTGTAC |
| | | CCTCTGGTGGACCCCAACAGCCAGTCTGGA |
| | | AAGGGAACAGCCAGCAGCGGCAGAAAGCCC |
| | | AGATGGTACAACCTGAAGATTGCCGGCGAT |
| | | CCCTCCTGGGAAGAAGAGAAGAAGAAGTGG |
| | | GAAGAAGATAAGAAAAAGGACCCGCTGGCC |
| | | AAGATCCTGGGCAAGCTGGCTGAGTACGGA |
| | | CTGATCCCTCTGTTCATCCCCTACACCGAC |
| | | AGCAACGAGCCCATCGTGAAAGAAATCAAG |
| | | TGGATGGAAAAGTCCCGGAACCAGAGCGTG |
| | | CGGCGGCTGGATAAGGACATGTTCATTCAG |
| | | GCCCTGGAACGGTTCCTGAGCTGGGAGAGC |
| | | TGGAACCTGAAAGTGAAAGAGGAATACGAG |
| | | AAGGTCGAGAAAGAGTACAAGACCCTGGAA |
| | | GAGAGGATCAAAGGAGGCTCTGGAGGAAGC |
| | | TCCGAAGTCGAGTTTTCCCATGAGTACTGG |
| | | ATGAGACACGCATTGACTCTCGCAAAGAGG |
| | | GCTCGAGATGAACGCGAGGTGCCCGTGGGG |
| | | GCAGTACTCGTGCTCAACAATCGCGTAATC |
| | | GGCGAAGGTTGGAATAGGGCAATCGGACTC |
| | | CACGACCCCACTGCACATGCGGAAATCATG |
| | | GCCCTTCGACAGGGAGGGCTTGTGATGCAG |
| | | AATTATCGACTTTATGATGCGACGCTGTAC |
| | | GTCACGTTTGAACCTTGCGTAATGTGCGCG |
| | | GGAGCTATGATTCACTCCCGCATTGGACGA |
| | | GTTGTATTCGGTGTTCGCAACGCCAAGACG |
| | | GGTGCCGCAGGTTCACTGATGGACGTGCTG |
| | | CATCATCCAGGCATGAACCACCGGGTAGAA |
| | | ATCACAGAAGGCATATTGGCGGACGAATGT |
| | | GCGGCGCTGTTGTGTCGTTTTTTTCGCATG |
| | | CCCAGGCGGGTCTTTAACGCCCAGAAAAAA |
| | | GCACAATCCTCTACTGACGGCTCTTCTGGA |
| | | TCTGAAACACCTGGCACAAGCGAGAGCGCC |
| | | ACCCCTGAGAGCTCTGGCGAGGACATCCAG |
| | | GCTCTGAAGGCTCTGGAACAGTATGAGAAA |
| | | GAGCGGCAAGAACAGCTGCTGCGGGACACC |
| | | CTGAACACCAACGAGTACCGGCTGAGCAAG |
| | | AGAGGCCTTAGAGGCTGGCGGGAAATCATC |
| | | CAGAAATGGCTGAAAATGGACGAGAACGAG |
| | | CCCTCCGAGAAGTACCTGGAAGTGTTCAAG |
| | | GACTACCAGCGGAAGCACCCTAGAGAGGCC |
| | | GGCGATTACAGCGTGTACGAGTTCCTGTCC |
| | | AAGAAAGAGAACCACTTCATCTGGCGGAAT |
| | | CACCCTGAGTACCCCTACCTGTACGCCACC |
| | | TTCTGCGAGATCGACAAGAAAAAGAAGGAC |
| | | GCCAAGCAGCAGGCCACCTTCACACTGGCC |
| | | GATCCTATCAATCACCCTCTGTGGGTCCGA |
| | | TTCGAGGAAAGAAGCGGCAGCAACCTGAAC |
| | | AAGTACAGAATCCTGACCGAGCAGCTGCAC |
| | | ACCGAGAAGCTGAAGAAAAAGCTGACAGTG |
| | | CAGCTGGACCGGCTGATCTACCCTACAGAA |
| | | TCTGGCGGCTGGGAAGAGAAGGGCAAAGTG |
| | | GACATTGTGCTGCTGCCCAGCCGGCAGTTC |
| | | TACAACCAGATCTTCCTGGACATCGAGGAA |
| | | AAGGGCAAGCACGCCTTCACCTACAAGGAT |
| | | GAGAGCATCAAGTTCCCTCTGAAGGGCACA |
| | | CTCGGCGGAGCCAGAGTGCAGTTCGACAGA |
| | | GATCACCTGAGAAGATACCCTCACAAGGTG |
| | | GAAAGCGGCAACGTGGGCAGAATCTACTTC |
| | | AACATGACCGTGAACATCGAGCCTACAGAG |
| | | TCCCCAGTGTCCAAGTCTCTGAAGATCCAC |
| | | CGGGACGACTTCCCCAAGGTGGTCAACTTC |
| | | AAGCCCAAAGAACTGACCGAGTGGATCAAG |
| | | GACAGCAAGGGCAAGAAACTGAAGTCCGGC |
| | | ATCGAGTCCCTGGAAATCGGCCTGAGAGTG |
| | | ATGAGCATCGACCTGGGACAGAGACAGGCC |
| | | GCTGCCGCCTCTATTTTCGAGGTGGTGGAT |
| | | CAGAAGCCCGACATCGAAGGCAAGCTGTTT |
| | | TTCCCAATCAAGGGCACCGAGCTGTATGCC |
| | | GTGCACAGAGCCAGCTTCAACATCAAGCTG |
| | | CCCGGCGAGACACTGGTCAAGAGCAGAGAA |
| | | GTGCTGCGGAAGGCCAGAGAGGACAATCTG |
| | | AAACTGATGAACCAGAAGCTCAACTTCCTG |
| | | CGGAACGTGCTGCACTTCCAGCAGTTCGAG |
| | | GACATCACCGAGAGAGAGAAGCGGGTCACC |
| | | AAGTGGATCAGCAGACAAGAGAACAGCGAC |
| | | GTGCCCCTGGTGTACCAGGATGAGCTGATC |
| | | CAGATCCGCGAGCTGATGTACAAGCCTTAC |
| | | AAGGACTGGGTCGCCTTCCTGAAGCAGCTC |
| | | CACAAGAGACTGGAAGTCGAGATCGGCAAA |
| | | GAAGTGAAGCACTGGCGGAAGTCCCTGAGC |
| | | GACGGAAGAAAGGGCCTGTACGGCATCTCC |
| | | CTGAAGAACATCGACGAGATCGATCGGACC |
| | | CGGAAGTTCCTGCTGAGATGGTCCCTGAGG |
| | | CCTACCGAACCTGGCGAAGTGCGTAGACTG |
| | | GAACCCGGCCAGAGATTCGCCATCGACCAG |
| | | CTGAATCACCTGAACGCCCTGAAAGAAGAT |
| | | CGGCTGAAGAAGATGGCCAACACCATCATC |
| | | ATGCACGCCCTGGGCTACTGCTACGACGTG |
| | | CGGAAGAAGAAATGGCAGGCTAAGAACCCC |
| | | GCCTGCCAGATCATCCTGTTCGAGGATCTG |
| | | AGCAACTACAACCCCTACGAGGAAAGGTCC |
| | | CGCTTCGAGAACAGCAAGCTCATGAAGTGG |
| | | TCCAGACGCGAGATCCCCAGACAGGTTGCA |
| | | CTGCAGGGCGAGATCTATGGCCTGCAAGTG |
| | | GGAGAAGTGGGCGCTCAGTTCAGCAGCAGA |
| | | TTCCACGCCAAGACAGGCAGCCCTGGCATC |
| | | AGATGTAGCGTCGTGACCAAAGAGAAGCTG |
| | | CAGGACAATCGGTTCTTCAAGAATCTGCAG |
| | | AGAGAGGGCAGACTGACCCTGGACAAAATC |
| | | GCCGTGCTGAAAGAGGGCGATCTGTACCCA |
| | | GACAAAGGCGGCGAGAAGTTCATCAGCCTG |
| | | AGCAAGGATCGGAAGTGCGTGACCACACAC |
| | | GCCGACATCAACGCCGCTCAGAACCTGCAG |
| | | AAGCGGTTCTGGACAAGAACCCACGGCTTC |
| | | TACAAGGTGTACTGCAAGGCCTACCAGGTG |
| | | GACGGCCAGACCGTGTACATCCCTGAGAGC |
| | | AAGGACCAGAAGCAGAAGATCATCGAAGAG |
| | | TTCGGCGAGGGCTACTTCATTCTGAAGGAC |
| | | GGGGTGTACGAATGGGTCAACGCCGGCAAG |
| | | CTGAAAATCAAGAAGGGCAGCTCCAAGCAG |
| | | AGCAGCAGCGAGCTGGTGGATAGCGACATC |
| | | CTGAAAGACAGCTTCGACCTGGCCTCCGAG |
| | | CTGAAAGGCGAAAAGCTGATGCTGTACAGG |
| | | GACCCCAGCGGCAATGTGTTCCCCAGCGAC |
| | | AAATGGATGGCCGCTGGCGTGTTCTTCGGA |
| | | AAGCTGGAACGCATCCTGATCAGCAAGCTG |
| | | ACCAACCAGTACTCCATCAGCACCATCGAG |
| | | GACGACAGCAGCAAGCAGTCTATGAAAAGG |
| | | CCGGCGGCCACGAAAAAGGCCGGCCAGGCA |
| | | AAAAAGAAAAAGGGATCCTACCCATACGAT |
| | | GTTCCAGATTACGCTTATCCCTACGACGTG |
| | | CCTGATTATGCATACCCATATGATGTCCCC |
| | | GACTATGCCTAA |
| BhCas12b | 277 | MAPKKKRKVGIHGVPAAATRSFILKIEPNE |
| GGSGGS- | | EVKKGLWKTHEVLNHGIAYYMNILKLIRQE |
| ABE8- | | AIYEHHEQDPKNPKKVSKAEIQAELWDFVL |
| Xten20 | | KMQKCNSFTHEVDKDEVFNILRELYEELVP |
| at K255 | | SSVEKKGEANQLSNKFLYPLVDPNSQSGKG |
| polypeptide | | TASSGRKPRWYNLKIAGDPSWEEEKKKWEE |
| | | DKKKDPLAKILGKLAEYGLIPLFIPYTDSN |
| | | EPIVKEIKWMEKSRNQSVRRLDKDMFIQAL |
| | | ERFLSWESWNLKVKEEYEKVEKEYKTLEER |
| | | IKGGSGGSSEVEFSHEYWMRHALTLAKRAR |
| | | DEREVPVGAVLVLNNRVIGEGWNRAIGLHD |
| | | PTAHAEIMALRQGGLVMQNYRLYDATLYVT |
| | | FEPCVMCAGAMIHSRIGRVVFGVRNAKTGA |
| | | AGSLMDVLHHPGMNHRVEITEGILADECAA |
| | | LLCRFFRMPRRVFNAQKKAQSSTDGSSGSE |
| | | TPGTSESATPESSGEDIQALKALEQYEKER |
| | | QEQLLRDTLNTNEYRLSKRGLRGWREIIQK |
| | | WLKMDENEPSEKYLEVFKDYQRKHPREAGD |
| | | YSVYEFLSKKENHFIWRNHPEYPYLYATFC |
| | | EIDKKKKDAKQQATFTLADPINHPLWVRFE |
| | | ERSGSNLNKYRILTEQLHTEKLKKKLTVQL |
| | | DRLIYPTESGGWEEKGKVDIVLLPSRQFYN |
| | | QIFLDIEEKGKHAFTYKDESIKFPLKGTLG |
| | | GARVQFDRDHLRRYPHKVESGNVGRIYFNM |
| | | TVNIEPTESPVSKSLKIHRDDFPKVVNFKP |
| | | KELTEWIKDSKGKKLKSGIESLEIGLRVMS |
| | | IDLGQRQAAAASIFEWDQKPDIEGKLFFPI |
| | | KGTELYAVHRASFNIKLPGETLVKSREVLR |
| | | KAREDNLKLMNQKLNFLRNVLHFQQFEDIT |
| | | EREKRVTKWISRQENSDVPLVYQDELIQIR |
| | | ELMYKPYKDWVAFLKQLHKRLEVEIGKEVK |
| | | HWRKSLSDGRKGLYGISLKNIDEIDRTRKF |
| | | LLRWSLRPTEPGEVRRLEPGQRFAIDQLNH |
| | | LNALKEDRLKKMANTIIMHALGYCYDVRKK |
| | | KWQAKNPACQIILFEDLSNYNPYEERSRFE |
| | | NSKLMKWSRREIPRQVALQGEIYGLQVGEV |
| | | GAQFSSRFHAKTGSPGIRCSVVTKEKLQDN |
| | | RFFKNLQREGRLTLDKIAVLKEGDLYPDKG |
| | | GEKFISLSKDRKCVTTHADINAAQNLQKRF |
| | | WTRTHGFYKVYCKAYQVDGQTVYIPESKDQ |
| | | KQKIIEEFGEGYFILKDGVYEWVNAGKLKI |
| | | KKGSSKQSSSELVDSDILKDSFDLASELKG |
| | | EKLMLYRDPSGNVFPSDKWMAAGVFFGKLE |
| | | RILISKLTNQYSISTIEDDSSKQSMKRPAA |
| | | TKKAGQAKKKKGSYPYDVPDYAYPYDVPDY |
| | | AYPYDVPDYA |
| BhCas12b | 278 | GCCACCATGGCCCCAAAGAAGAAGCGGAAG |
| GGSGGS- | | GTCGGTATCCACGGAGTCCCAGCAGCCGCC |
| ABE8- | | ACCAGATCCTTCATCCTGAAGATCGAGCCC |
| Xten20 | | AACGAGGAAGTGAAGAAAGGCCTCTGGAAA |
| at D306 | | ACCCACGAGGTGCTGAACCACGGAATCGCC |
| poly- | | TACTACATGAATATCCTGAAGCTGATCCGG |
| nucleotide | | CAAGAGGCCATCTACGAGCACCACGAGCAG |
| | | GACCCCAAGAATCCCAAGAAGGTGTCCAAG |
| | | GCCGAGATCCAGGCCGAGCTGTGGGATTTC |
| | | GTGCTGAAGATGCAGAAGTGCAACAGCTTC |
| | | ACACACGAGGTGGACAAGGACGAGGTGTTC |
| | | AACATCCTGAGAGAGCTGTACGAGGAACTG |
| | | GTGCCCAGCAGCGTGGAAAAGAAGGGCGAA |
| | | GCCAACCAGCTGAGCAACAAGTTTCTGTAC |
| | | CCTCTGGTGGACCCCAACAGCCAGTCTGGA |
| | | AAGGGAACAGCCAGCAGCGGCAGAAAGCCC |
| | | AGATGGTACAACCTGAAGATTGCCGGCGAT |
| | | CCCTCCTGGGAAGAAGAGAAGAAGAAGTGG |
| | | GAAGAAGATAAGAAAAAGGACCCGCTGGCC |
| | | AAGATCCTGGGCAAGCTGGCTGAGTACGGA |
| | | CTGATCCCTCTGTTCATCCCCTACACCGAC |
| | | AGCAACGAGCCCATCGTGAAAGAAATCAAG |
| | | TGGATGGAAAAGTCCCGGAACCAGAGCGTG |
| | | CGGCGGCTGGATAAGGACATGTTCATTCAG |
| | | GCCCTGGAACGGTTCCTGAGCTGGGAGAGC |
| | | TGGAACCTGAAAGTGAAAGAGGAATACGAG |
| | | AAGGTCGAGAAAGAGTACAAGACCCTGGAA |
| | | GAGAGGATCAAAGAGGACATCCAGGCTCTG |
| | | AAGGCTCTGGAACAGTATGAGAAAGAGCGG |
| | | CAAGAACAGCTGCTGCGGGACACCCTGAAC |
| | | ACCAACGAGTACCGGCTGAGCAAGAGAGGC |
| | | CTTAGAGGCTGGCGGGAAATCATCCAGAAA |
| | | TGGCTGAAAATGGACGGAGGCTCTGGAGGA |
| | | AGCTCCGAAGTCGAGTTTTCCCATGAGTAC |
| | | TGGATGAGACACGCATTGACTCTCGCAAAG |
| | | AGGGCTCGAGATGAACGCGAGGTGCCCGTG |
| | | GGGGCAGTACTCGTGCTCAACAATCGCGTA |
| | | ATCGGCGAAGGTTGGAATAGGGCAATCGGA |
| | | CTCCACGACCCCACTGCACATGCGGAAATC |
| | | ATGGCCCTTCGACAGGGAGGGCTTGTGATG |
| | | CAGAATTATCGACTTTATGATGCGACGCTG |
| | | TACGTCACGTTTGAACCTTGCGTAATGTGC |
| | | GCGGGAGCTATGATTCACTCCCGCATTGGA |
| | | CGAGTTGTATTCGGTGTTCGCAACGCCAAG |
| | | ACGGGTGCCGCAGGTTCACTGATGGACGTG |
| | | CTGCATCATCCAGGCATGAACCACCGGGTA |
| | | GAAATCACAGAAGGCATATTGGCGGACGAA |
| | | TGTGCGGCGCTGTTGTGTCGTTTTTTTCGC |
| | | ATGCCCAGGCGGGTCTTTAACGCCCAGAAA |
| | | AAAGCACAATCCTCTACTGACGGCTCTTCT |
| | | GGATCTGAAACACCTGGCACAAGCGAGAGC |
| | | GCCACCCCTGAGAGCTCTGGCGAGAACGAG |
| | | CCCTCCGAGAAGTACCTGGAAGTGTTCAAG |
| | | GACTACCAGCGGAAGCACCCTAGAGAGGCC |
| | | GGCGATTACAGCGTGTACGAGTTCCTGTCC |
| | | AAGAAAGAGAACCACTTCATCTGGCGGAAT |
| | | CACCCTGAGTACCCCTACCTGTACGCCACC |
| | | TTCTGCGAGATCGACAAGAAAAAGAAGGAC |
| | | GCCAAGCAGCAGGCCACCTTCACACTGGCC |
| | | GATCCTATCAATCACCCTCTGTGGGTCCGA |
| | | TTCGAGGAAAGAAGCGGCAGCAACCTGAAC |
| | | AAGTACAGAATCCTGACCGAGCAGCTGCAC |
| | | ACCGAGAAGCTGAAGAAAAAGCTGACAGTG |
| | | CAGCTGGACCGGCTGATCTACCCTACAGAA |
| | | TCTGGCGGCTGGGAAGAGAAGGGCAAAGTG |
| | | GACATTGTGCTGCTGCCCAGCCGGCAGTTC |
| | | TACAACCAGATCTTCCTGGACATCGAGGAA |
| | | AAGGGCAAGCACGCCTTCACCTACAAGGAT |
| | | GAGAGCATCAAGTTCCCTCTGAAGGGCACA |
| | | CTCGGCGGAGCCAGAGTGCAGTTCGACAGA |
| | | GATCACCTGAGAAGATACCCTCACAAGGTG |
| | | GAAAGCGGCAACGTGGGCAGAATCTACTTC |
| | | AACATGACCGTGAACATCGAGCCTACAGAG |
| | | TCCCCAGTGTCCAAGTCTCTGAAGATCCAC |
| | | CGGGACGACTTCCCCAAGGTGGTCAACTTC |
| | | AAGCCCAAAGAACTGACCGAGTGGATCAAG |
| | | GACAGCAAGGGCAAGAAACTGAAGTCCGGC |
| | | ATCGAGTCCCTGGAAATCGGCCTGAGAGTG |
| | | ATGAGCATCGACCTGGGACAGAGACAGGCC |
| | | GCTGCCGCCTCTATTTTCGAGGTGGTGGAT |
| | | CAGAAGCCCGACATCGAAGGCAAGCTGTTT |
| | | TTCCCAATCAAGGGCACCGAGCTGTATGCC |
| | | GTGCACAGAGCCAGCTTCAACATCAAGCTG |
| | | CCCGGCGAGACACTGGTCAAGAGCAGAGAA |
| | | GTGCTGCGGAAGGCCAGAGAGGACAATCTG |
| | | AAACTGATGAACCAGAAGCTCAACTTCCTG |
| | | CGGAACGTGCTGCACTTCCAGCAGTTCGAG |
| | | GACATCACCGAGAGAGAGAAGCGGGTCACC |
| | | AAGTGGATCAGCAGACAAGAGAACAGCGAC |
| | | GTGCCCCTGGTGTACCAGGATGAGCTGATC |
| | | CAGATCCGCGAGCTGATGTACAAGCCTTAC |
| | | AAGGACTGGGTCGCCTTCCTGAAGCAGCTC |
| | | CACAAGAGACTGGAAGTCGAGATCGGCAAA |
| | | GAAGTGAAGCACTGGCGGAAGTCCCTGAGC |
| | | GACGGAAGAAAGGGCCTGTACGGCATCTCC |
| | | CTGAAGAACATCGACGAGATCGATCGGACC |
| | | CGGAAGTTCCTGCTGAGATGGTCCCTGAGG |
| | | CCTACCGAACCTGGCGAAGTGCGTAGACTG |
| | | GAACCCGGCCAGAGATTCGCCATCGACCAG |
| | | CTGAATCACCTGAACGCCCTGAAAGAAGAT |
| | | CGGCTGAAGAAGATGGCCAACACCATCATC |
| | | ATGCACGCCCTGGGCTACTGCTACGACGTG |
| | | CGGAAGAAGAAATGGCAGGCTAAGAACCCC |
| | | GCCTGCCAGATCATCCTGTTCGAGGATCTG |
| | | AGCAACTACAACCCCTACGAGGAAAGGTCC |
| | | CGCTTCGAGAACAGCAAGCTCATGAAGTGG |
| | | TCCAGACGCGAGATCCCCAGACA |
| | | GGTTGCACTGCAGGGCGAGATCTATGGCCT |
| | | GCAAGTGGGAGAAGTGGGCGCTCAGTTCAG |
| | | CAGCAGATTCCACGCCAAGACAGGCAGCCC |
| | | TGGCATCAGATGTAGCGTCGTGACCAAAGA |
| | | GAAGCTGCAGGACAATCGGTTCTTCAAGAA |
| | | TCTGCAGAGAGAGGGCAGACTGACCCTGGA |
| | | CAAAATCGCCGTGCTGAAAGAGGGCGATCT |
| | | GTACCCAGACAAAGGCGGCGAGAAGTTCAT |
| | | CAGCCTGAGCAAGGATCGGAAGTGCGTGAC |
| | | CACACACGCCGACATCAACGCCGCTCAGAA |
| | | CCTGCAGAAGCGGTTCTGGACAAGAACCCA |
| | | CGGCTTCTACAAGGTGTACTGCAAGGCCTA |
| | | CCAGGTGGACGGCCAGACCGTGTACATCCC |
| | | TGAGAGCAAGGACCAGAAGCAGAAGATCAT |
| | | CGAAGAGTTCGGCGAGGGCTACTTCATTCT |
| | | GAAGGACGGGGTGTACGAATGGGTCAACGC |
| | | CGGCAAGCTGAAAATCAAGAAGGGCAGCTC |
| | | CAAGCAGAGCAGCAGCGAGCTGGTGGATAG |
| | | CGACATCCTGAAAGACAGCTTCGACCTGGC |
| | | CTCCGAGCTGAAAGGCGAAAAGCTGATGCT |
| | | GTACAGGGACCCCAGCGGCAATGTGTTCCC |
| | | CAGCGACAAATGGATGGCCGCTGGCGTGTT |
| | | CTTCGGAAAGCTGGAACGCATCCTGATCAG |
| | | CAAGCTGACCAACCAGTACTCCATCAGCAC |
| | | CATCGAGGACGACAGCAGCAAGCAGTCTAT |
| | | GAAAAGGCCGGCGGCCACGAAAAAGGCCGG |
| | | CCAGGCAAAAAAGAAAAAGGGATCCTACCC |
| | | ATACGATGTTCCAGATTACGCTTATCCCTA |
| | | CGACGTGCCTGATTATGCATACCCATATGA |
| | | TGTCCCCGACTATGCCTAA |
| BhCas12b | 279 | MAPKKKRKVGIHGVPAAATRSFILKIEPNE |
| GGSGGS- | | EVKKGLWKTHEVLNHGIAYYMNILKLIRQE |
| ABE8- | | AIYEHHEQDPKNPKKVSKAEIQAELWDFVL |
| Xten20 | | KMQKCNSFTHEVDKDEVFNILRELYEELVP |
| at D306 | | SSVEKKGEANQLSNKFLYPLVDPNSQSGKG |
| polypeptide | | TASSGRKPRWYNLKIAGDPSWEEEKKKWEE |
| | | DKKKDPLAKILGKLAEYGLIPLFIPYTDSN |
| | | EPIVKEIKWMEKSRNQSVRRLDKDMFIQAL |
| | | ERFLSWESWNLKVKEEYEKVEKEYKTLEER |
| | | IKEDIQALKALEQYEKERQEQLLRDTLNTN |
| | | EYRLSKRGLRGWREIIQKWLKMDGGSGGSS |
| | | EVEFSHEYWMRHALTLAKRARDEREVPVGA |
| | | VLVLNNRVIGEGWNRAIGLHDPTAHAEIMA |
| | | LRQGGLVMQNYRLYDATLYVTFEPCVMCAG |
| | | AMIHSRIGRVVFGVRNAKTGAAGSLMDVLH |
| | | HPGMNHRVEITEGILADECAALLCRFFRMP |
| | | RRVFNAQKKAQSSTDGSSGSETPGTSESAT |
| | | PESSGENEPSEKYLEVFKDYQRKHPREAGD |
| | | YSVYEFLSKKENHFIWRNHPEYPYLYATFC |
| | | EIDKKKKDAKQQATFTLADPINHPLWVRFE |
| | | ERSGSNLNKYRILTEQLHTEKLKKKLTVQL |
| | | DRLIYPTESGGWEEKGKVDIVLLPSRQFYN |
| | | QIFLDIEEKGKHAFTYKDESIKFPLKGTLG |
| | | GARVQFDRDHLRRYPHKVESGNVGRIYFNM |
| | | TVNIEPTESPVSKSLKIHRDDFPKWNFKPK |
| | | ELTEWIKDSKGKKLKSGIESLEIGLRVMSI |
| | | DLGQRQAAAASIFEVVDQKPDIEGKLFFPI |
| | | KGTELYAVHRASFNIKLPGETLVKSREVLR |
| | | KAREDNLKLMNQKLNFLRNVLHFQQFEDIT |
| | | EREKRVTKWISRQENSDVPLVYQDELIQIR |
| | | ELMYKPYKDWVAFLKQLHKRLEVEIGKEVK |
| | | HWRKSLSDGRKGLYGISLKNIDEIDRTRKF |
| | | LLRWSLRPTEPGEVRRLEPGQRFAIDQLNH |
| | | LNALKEDRLKKMANTIIMHALGYCYDVRKK |
| | | KWQAKNPACQIILFEDLSNYNPYEERSRFE |
| | | NSKLMKWSRREIPRQVALQGEIYGLQVGEV |
| | | GAQFSSRFHAKTGSPGIRCSWTKEKLQDNR |
| | | FFKNLQREGRLTLDKIAVLKEGDLYPDKGG |
| | | EKFISLSKDRKCVTTHADINAAQNLQKRFW |
| | | TRTHGFYKVYCKAYQVDGQTVYIPESKDQK |
| | | QKIIEEFGEGYFILKDGVYEWVNAGKLKIK |
| | | KGSSKQSSSELVDSDILKDSFDLASELKGE |
| | | KLMLYRDPSGNVFPSDKWMAAGVFFGKLER |
| | | ILISKLTNQYSISTIEDDSSKQSMKRPAAT |
| | | KKAGQAKKKKGSYPYDVPDYAYPYDVPDYA |
| | | YPYDVPDYA |
| BhCas12b | 280 | GCCACCATGGCCCCAAAGAAGAAGCGGAAG |
| GGSGGS- | | GTCGGTATCCACGGAGTCCCAGCAGCCGCC |
| ABE8- | | ACCAGATCCTTCATCCTGAAGATCGAGCCC |
| Xten20 | | AACGAGGAAGTGAAGAAAGGCCTCTGGAAA |
| at D980 | | ACCCACGAGGTGCTGAACCACGGAATCGCC |
| poly- | | TACTACATGAATATCCTGAAGCTGATCCGG |
| nucleotide | | CAAGAGGCCATCTACGAGCACCACGAGCAG |
| | | GACCCCAAGAATCCCAAGAAGGTGTCCAAG |
| | | GCCGAGATCCAGGCCGAGCTGTGGGATTTC |
| | | GTGCTGAAGATGCAGAAGTGCAACAGCTTC |
| | | ACACACGAGGTGGACAAGGACGAGGTGTTC |
| | | AACATCCTGAGAGAGCTGTACGAGGAACTG |
| | | GTGCCCAGCAGCGTGGAAAAGAAGGGCGAA |
| | | GCCAACCAGCTGAGCAACAAGTTTCTGTAC |
| | | CCTCTGGTGGACCCCAACAGCCAGTCTGGA |
| | | AAGGGAACAGCCAGCAGCGGCAGAAAGCCC |
| | | AGATGGTACAACCTGAAGATTGCCGGCGAT |
| | | CCCTCCTGGGAAGAAGAGAAGAAGAAGTGG |
| | | GAAGAAGATAAGAAAAAGGACCCGCTGGCC |
| | | AAGATCCTGGGCAAGCTGGCTGAGTACGGA |
| | | CTGATCCCTCTGTTCATCCCCTACACCGAC |
| | | AGCAACGAGCCCATCGTGAAAGAAATCAAG |
| | | TGGATGGAAAAGTCCCGGAACCAGAGCGTG |
| | | CGGCGGCTGGATAAGGACATGTTCATTCAG |
| | | GCCCTGGAACGGTTCCTGAGCTGGGAGAGC |
| | | TGGAACCTGAAAGTGAAAGAGGAATACGAG |
| | | AAGGTCGAGAAAGAGTACAAGACCCTGGAA |
| | | GAGAGGATCAAAGAGGACATCCAGGCTCTG |
| | | AAGGCTCTGGAACAGTATGAGAAAGAGCGG |
| | | CAAGAACAGCTGCTGCGGGACACCCTGAAC |
| | | ACCAACGAGTACCGGCTGAGCAAGAGAGGC |
| | | CTTAGAGGCTGGCGGGAAATCATCCAGAAA |
| | | TGGCTGAAAATGGACGAGAACGAGCCCTCC |
| | | GAGAAGTACCTGGAAGTGTTCAAGGACTAC |
| | | CAGCGGAAGCACCCTAGAGAGGCCGGCGAT |
| | | TACAGCGTGTACGAGTTCCTGTCCAAGAAA |
| | | GAGAACCACTTCATCTGGCGGAATCACCCT |
| | | GAGTACCCCTACCTGTACGCCACCTTCTGC |
| | | GAGATCGACAAGAAAAAGAAGGACGCCAAG |
| | | CAGCAGGCCACCTTCACACTGGCCGATCCT |
| | | ATCAATCACCCTCTGTGGGTCCGATTCGAG |
| | | GAAAGAAGCGGCAGCAACCTGAACAAGTAC |
| | | AGAATCCTGACCGAGCAGCTGCACACCGAG |
| | | AAGCTGAAGAAAAAGCTGACAGTGCAGCTG |
| | | GACCGGCTGATCTACCCTACAGAATCTGGC |
| | | GGCTGGGAAGAGAAGGGCAAAGTGGACATT |
| | | GTGCTGCTGCCCAGCCGGCAGTTCTACAAC |
| | | CAGATCTTCCTGGACATCGAGGAAAAGGGC |
| | | AAGCACGCCTTCACCTACAAGGATGAGAGC |
| | | ATCAAGTTCCCTCTGAAGGGCACACTCGGC |
| | | GGAGCCAGAGTGCAGTTCGACAGAGATCAC |
| | | CTGAGAAGATACCCTCACAAGGTGGAAAGC |
| | | GGCAACGTGGGCAGAATCTACTTCAACATG |
| | | ACCGTGAACATCGAGCCTACAGAGTCCCCA |
| | | GTGTCCAAGTCTCTGAAGATCCACCGGGAC |
| | | GACTTCCCCAAGGTGGTCAACTTCAAGCCC |
| | | AAAGAACTGACCGAGTGGATCAAGGACAGC |
| | | AAGGGCAAGAAACTGAAGTCCGGCATCGAG |
| | | TCCCTGGAAATCGGCCTGAGAGTGATGAGC |
| | | ATCGACCTGGGACAGAGACAGGCCGCTGCC |
| | | GCCTCTATTTTCGAGGTGGTGGATCAGAAG |
| | | CCCGACATCGAAGGCAAGCTGTTTTTCCCA |
| | | ATCAAGGGCACCGAGCTGTATGCCGTGCAC |
| | | AGAGCCAGCTTCAACATCAAGCTGCCCGGC |
| | | GAGACACTGGTCAAGAGCAGAGAAGTGCTG |
| | | CGGAAGGCCAGAGAGGACAATCTGAAACTG |
| | | ATGAACCAGAAGCTCAACTTCCTGCGGAAC |
| | | GTGCTGCACTTCCAGCAGTTCGAGGACATC |
| | | ACCGAGAGAGAGAAGCGGGTCACCAAGTGG |
| | | ATCAGCAGACAAGAGAACAGCGACGTGCCC |
| | | CTGGTGTACCAGGATGAGCTGATCCAGATC |
| | | CGCGAGCTGATGTACAAGCCTTACAAGGAC |
| | | TGGGTCGCCTTCCTGAAGCAGCTCCACAAG |
| | | AGACTGGAAGTCGAGATCGGCAAAGAAGTG |
| | | AAGCACTGGCGGAAGTCCCTGAGCGACGGA |
| | | AGAAAGGGCCTGTACGGCATCTCCCTGAAG |
| | | AACATCGACGAGATCGATCGGACCCGGAAG |
| | | TTCCTGCTGAGATGGTCCCTGAGGCCTACC |
| | | GAACCTGGCGAAGTGCGTAGACTGGAACCC |
| | | GGCCAGAGATTCGCCATCGACCAGCTGAAT |
| | | CACCTGAACGCCCTGAAAGAAGATCGGCTG |
| | | AAGAAGATGGCCAACACCATCATCATGCAC |
| | | GCCCTGGGCTACTGCTACGACGTGCGGAAG |
| | | AAGAAATGGCAGGCTAAGAACCCCGCCTGC |
| | | CAGATCATCCTGTTCGAGGATCTGAGCAAC |
| | | TACAACCCCTACGAGGAAAGGTCCCGCTTC |
| | | GAGAACAGCAAGCTCATGAAGTGGTCCAGA |
| | | CGCGAGATCCCCAGACAGGTTGCACTGCAG |
| | | GGCGAGATCTATGGCCTGCAAGTGGGAGAA |
| | | GTGGGCGCTCAGTTCAGCAGCAGATTCCAC |
| | | GCCAAGACAGGCAGCCCTGGCATCAGATGT |
| | | AGCGTCGTGACCAAAGAGAAGCTGCAGGAC |
| | | AATCGGTTCTTCAAGAATCTGCAGAGAGAG |
| | | GGCAGACTGACCCTGGACAAAATCGCCGTG |
| | | CTGAAAGAGGGCGATCTGTACCCAGACAAA |
| | | GGCGGCGAGAAGTTCATCAGCCTGAGCAAG |
| | | GATCGGAAGTGCGTGAC |
| | | CACACACGCCGACATCAACGCCGCTCAGAA |
| | | CCTGCAGAAGCGGTTCTGGACAAGAACCCA |
| | | CGGCTTCTACAAGGTGTACTGCAAGGCCTA |
| | | CCAGGTGGACGGAGGCTCTGGAGGAAGCTC |
| | | CGAAGTCGAGTTTTCCCATGAGTACTGGAT |
| | | GAGACACGCATTGACTCTCGCAAAGAGGGC |
| | | TCGAGATGAACGCGAGGTGCCCGTGGGGGC |
| | | AGTACTCGTGCTCAACAATCGCGTAATCGG |
| | | CGAAGGTTGGAATAGGGCAATCGGACTCCA |
| | | CGACCCCACTGCACATGCGGAAATCATGGC |
| | | CCTTCGACAGGGAGGGCTTGTGATGCAGAA |
| | | TTATCGACTTTATGATGCGACGCTGTACGT |
| | | CACGTTTGAACCTTGCGTAATGTGCGCGGG |
| | | AGCTATGATTCACTCCCGCATTGGACGAGT |
| | | TGTATTCGGTGTTCGCAACGCCAAGACGGG |
| | | TGCCGCAGGTTCACTGATGGACGTGCTGCA |
| | | TCATCCAGGCATGAACCACCGGGTAGAAAT |
| | | CACAGAAGGCATATTGGCGGACGAATGTGC |
| | | GGCGCTGTTGTGTCGTTTTTTTCGCATGCC |
| | | CAGGCGGGTCTTTAACGCCCAGAAAAAAGC |
| | | ACAATCCTCTACTGACGGCTCTTCTGGATC |
| | | TGAAACACCTGGCACAAGCGAGAGCGCCAC |
| | | CCCTGAGAGCTCTGGCGGCCAGACCGTGTA |
| | | CATCCCTGAGAGCAAGGACCAGAAGCAGAA |
| | | GATCATCGAAGAGTTCGGCGAGGGCTACTT |
| | | CATTCTGAAGGACGGGGTGTACGAATGGGT |
| | | CAACGCCGGCAAGCTGAAAATCAAGAAGGG |
| | | CAGCTCCAAGCAGAGCAGCAGCGAGCTGGT |
| | | GGATAGCGACATCCTGAAAGACAGCTTCGA |
| | | CCTGGCCTCCGAGCTGAAAGGCGAAAAGCT |
| | | GATGCTGTACAGGGACCCCAGCGGCAATGT |
| | | GTTCCCCAGCGACAAATGGATGGCCGCTGG |
| | | CGTGTTCTTCGGAAAGCTGGAACGCATCCT |
| | | GATCAGCAAGCTGACCAACCAGTACTCCAT |
| | | CAGCACCATCGAGGACGACAGCAGCAAGCA |
| | | GTCTATGAAAAGGCCGGCGGCCACGAAAAA |
| | | GGCCGGCCAGGCAAAAAAGAAAAAGGGATC |
| | | CTACCCATACGATGTTCCAGATTACGCTTA |
| | | TCCCTACGACGTGCCTGATTATGCATACCC |
| | | ATATGATGTCCCCGACTATGCCTAA |
| BhCas12b | 281 | MAPKKKRKVGIHGVPAAATRSFILKIEPNE |
| GGSGGS- | | EVKKGLWKTHEVLNHGIAYYMNILKLIRQE |
| ABE8- | | AIYEHHEQDPKNPKKVSKAEIQAELWDFVL |
| Xten20 | | KMQKCNSFTHEVDKDEVFNILRELYEELVP |
| at D980 | | SSVEKKGEANQLSNKFLYPLVDPNSQSGKG |
| polypeptide | | TASSGRKPRWYNLKIAGDPSWEEEKKKWEE |
| | | DKKKDPLAKILGKLAEYGLIPLFIPYTDSN |
| | | EPIVKEIKWMEKSRNQSVRRLDKDMFIQAL |
| | | ERFLSWESWNLKVKEEYEKVEKEYKTLEER |
| | | IKEDIQALKALEQYEKERQEQLLRDTLNTN |
| | | EYRLSKRGLRGWREIIQKWLKMDENEPSEK |
| | | YLEVFKDYQRKHPREAGDYSVYEFLSKKEN |
| | | HFIWRNHPEYPYLYATFCEIDKKKKDAKQQ |
| | | ATFTLADPINHPLWVRFEERSGSNLNKYRI |
| | | LTEQLHTEKLKKKLTVQLDRLIYPTESGGW |
| | | EEKGKVDIVLLPSRQFYNQIFLDIEEKGKH |
| | | AFTYKDESIKFPLKGTLGGARVQFDRDHLR |
| | | RYPHKVESGNVGRIYFNMTVNIEPTESPVS |
| | | KSLKIHRDDFPKVVNFKPKELTEWIKDSKG |
| | | KKLKSGIESLEIGLRVMSIDLGQRQAAAAS |
| | | IFEWDQKPDIEGKLFFPIKGTELYAVHRAS |
| | | FNIKLPGETLVKSREVLRKAREDNLKLMNQ |
| | | KLNFLRNVLHFQQFEDITEREKRVTKWISR |
| | | QENSDVPLVYQDELIQIRELMYKPYKDWVA |
| | | FLKQLHKRLEVEIGKEVKHWRKSLSDGRKG |
| | | LYGISLKNIDEIDRTRKFLLRWSLRPTEPG |
| | | EVRRLEPGQRFAIDQLNHLNALKEDRLKKM |
| | | ANTIIMHALGYCYDVRKKKWQAKNPACQII |
| | | LFEDLSNYNPYEERSRFENSKLMKWSRREI |
| | | PRQVALQGEIYGLQVGEVGAQFSSRFHAKT |
| | | GSPGIRCSWTKEKLQDNRFFKNLQREGRLT |
| | | LDKIAVLKEGDLYPDKGGEKFISLSKDRKC |
| | | VTTHADINAAQNLQKRFWTRTHGFYKVYCK |
| | | AYQVDGGSGGSSEVEFSHEYWMRHALTLAK |
| | | RARDEREVPVGAVLVLNNRVIGEGWNRAIG |
| | | LHDPTAHAEIMALRQGGLVMQNYRLYDATL |
| | | YVTFEPCVMCAGAMIHSRIGRWFGVRNAKT |
| | | GAAGSLMDVLHHPGMNHRVEITEGILADEC |
| | | AALLCRFFRMPRRVFNAQKKAQSSTDGSSG |
| | | SETPGTSESATPESSGGQTVYIPESKDQKQ |
| | | KIIEEFGEGYFILKDGVYEWVNAGKLKIKK |
| | | GSSKQSSSELVDSDILKDSFDLASELKGEK |
| | | LMLYRDPSGNVFPSDKWMAAGVFFGKLERI |
| | | LISKLTNQYSISTIEDDSSKQSMKRPAATK |
| | | KAGQAKKKKGSYPYDVPDYAYPYDVPDYAY |
| | | PYDVPDYA |
| BhCas12b | 282 | GCCACCATGGCCCCAAAGAAGAAGCGGAAG |
| GGSGGS- | | GTCGGTATCCACGGAGTCCCAGCAGCCGCC |
| ABE8- | | ACCAGATCCTTCATCCTGAAGATCGAGCCC |
| Xten20 | | AACGAGGAAGTGAAGAAAGGCCTCTGGAAA |
| atK1019 | | ACCCACGAGGTGCTGAACCACGGAATCGCC |
| poly- | | TACTACATGAATATCCTGAAGCTGATCCGG |
| nucleotide | | CAAGAGGCCATCTACGAGCACCACGAGCAG |
| | | GACCCCAAGAATCCCAAGAAGGTGTCCAAG |
| | | GCCGAGATCCAGGCCGAGCTGTGGGATTTC |
| | | GTGCTGAAGATGCAGAAGTGCAACAGCTTC |
| | | ACACACGAGGTGGACAAGGACGAGGTGTTC |
| | | AACATCCTGAGAGAGCTGTACGAGGAACTG |
| | | GTGCCCAGCAGCGTGGAAAAGAAGGGCGAA |
| | | GCCAACCAGCTGAGCAACAAGTTTCTGTAC |
| | | CCTCTGGTGGACCCCAACAGCCAGTCTGGA |
| | | AAGGGAACAGCCAGCAGCGGCAGAAAGCCC |
| | | AGATGGTACAACCTGAAGATTGCCGGCGAT |
| | | CCCTCCTGGGAAGAAGAGAAGAAGAAGTGG |
| | | GAAGAAGATAAGAAAAAGGACCCGCTGGCC |
| | | AAGATCCTGGGCAAGCTGGCTGAGTACGGA |
| | | CTGATCCCTCTGTTCATCCCCTACACCGAC |
| | | AGCAACGAGCCCATCGTGAAAGAAATCAAG |
| | | TGGATGGAAAAGTCCCGGAACCAGAGCGTG |
| | | CGGCGGCTGGATAAGGACATGTTCATTCAG |
| | | GCCCTGGAACGGTTCCTGAGCTGGGAGAGC |
| | | TGGAACCTGAAAGTGAAAGAGGAATACGAG |
| | | AAGGTCGAGAAAGAGTACAAGACCCTGGAA |
| | | GAGAGGATCAAAGAGGACATCCAGGCTCTG |
| | | AAGGCTCTGGAACAGTATGAGAAAGAGCGG |
| | | CAAGAACAGCTGCTGCGGGACACCCTGAAC |
| | | ACCAACGAGTACCGGCTGAGCAAGAGAGGC |
| | | CTTAGAGGCTGGCGGGAAATCATCCAGAAA |
| | | TGGCTGAAAATGGACGAGAACGAGCCCTCC |
| | | GAGAAGTACCTGGAAGTGTTCAAGGACTAC |
| | | CAGCGGAAGCACCCTAGAGAGGCCGGCGAT |
| | | TACAGCGTGTACGAGTTCCTGTCCAAGAAA |
| | | GAGAACCACTTCATCTGGCGGAATCACCCT |
| | | GAGTACCCCTACCTGTACGCCACCTTCTGC |
| | | GAGATCGACAAGAAAAAGAAGGACGCCAAG |
| | | CAGCAGGCCACCTTCACACTGGCCGATCCT |
| | | ATCAATCACCCTCTGTGGGTCCGATTCGAG |
| | | GAAAGAAGCGGCAGCAACCTGAACAAGTAC |
| | | AGAATCCTGACCGAGCAGCTGCACACCGAG |
| | | AAGCTGAAGAAAAAGCTGACAGTGCAGCTG |
| | | GACCGGCTGATCTACCCTACAGAATCTGGC |
| | | GGCTGGGAAGAGAAGGGCAAAGTGGACATT |
| | | GTGCTGCTGCCCAGCCGGCAGTTCTACAAC |
| | | CAGATCTTCCTGGACATCGAGGAAAAGGGC |
| | | AAGCACGCCTTCACCTACAAGGATGAGAGC |
| | | ATCAAGTTCCCTCTGAAGGGCACACTCGGC |
| | | GGAGCCAGAGTGCAGTTCGACAGAGATCAC |
| | | CTGAGAAGATACCCTCACAAGGTGGAAAGC |
| | | GGCAACGTGGGCAGAATCTACTTCAACATG |
| | | ACCGTGAACATCGAGCCTACAGAGTCCCCA |
| | | GTGTCCAAGTCTCTGAAGATCCACCGGGAC |
| | | GACTTCCCCAAGGTGGTCAACTTCAAGCCC |
| | | AAAGAACTGACCGAGTGGATCAAGGACAGC |
| | | AAGGGCAAGAAACTGAAGTCCGGCATCGAG |
| | | TCCCTGGAAATCGGCCTGAGAGTGATGAGC |
| | | ATCGACCTGGGACAGAGACAGGCCGCTGCC |
| | | GCCTCTATTTTCGAGGTGGTGGATCAGAAG |
| | | CCCGACATCGAAGGCAAGCTGTTTTTCCCA |
| | | ATCAAGGGCACCGAGCTGTATGCCGTGCAC |
| | | AGAGCCAGCTTCAACATCAAGCTGCCCGGC |
| | | GAGACACTGGTCAAGAGCAGAGAAGTGCTG |
| | | CGGAAGGCCAGAGAGGACAATCTGAAACTG |
| | | ATGAACCAGAAGCTCAACTTCCTGCGGAAC |
| | | GTGCTGCACTTCCAGCAGTTCGAGGACATC |
| | | ACCGAGAGAGAGAAGCGGGTCACCAAGTGG |
| | | ATCAGCAGACAAGAGAACAGCGACGTGCCC |
| | | CTGGTGTACCAGGATGAGCTGATCCAGATC |
| | | CGCGAGCTGATGTACAAGCCTTACAAGGAC |
| | | TGGGTCGCCTTCCTGAAGCAGCTCCACAAG |
| | | AGACTGGAAGTCGAGATCGGCAAAGAAGTG |
| | | AAGCACTGGCGGAAGTCCCTGAGCGACGGA |
| | | AGAAAGGGCCTGTACGGCATCTCCCTGAAG |
| | | AACATCGACGAGATCGATCGGACCCGGAAG |
| | | TTCCTGCTGAGATGGTCCCTGAGGCCTACC |
| | | GAACCTGGCGAAGTGCGTAGACTGGAACCC |
| | | GGCCAGAGATTCGCCATCGACCAGCTGAAT |
| | | CACCTGAACGCCCTGAAAGAAGATCGGCTG |
| | | AAGAAGATGGCCAACACCATCATCATGCAC |
| | | GCCCTGGGCTACTGCTACGACGTGCGGAAG |
| | | AAGAAATGGCAGGCTAAGAACCCCGCCTGC |
| | | CAGATCATCCTGTTCGAGGATCTGAGCAAC |
| | | TAC |
| | | AACCCCTACGAGGAAAGGTCCCGCTTCGAG |
| | | AACAGCAAGCTCATGAAGTGGTCCAGACGC |
| | | GAGATCCCCAGACAGGTTGCACTGCAGGGC |
| | | GAGATCTATGGCCTGCAAGTGGGAGAAGTG |
| | | GGCGCTCAGTTCAGCAGCAGATTCCACGCC |
| | | AAGACAGGCAGCCCTGGCATCAGATGTAGC |
| | | GTCGTGACCAAAGAGAAGCTGCAGGACAAT |
| | | CGGTTCTTCAAGAATCTGCAGAGAGAGGGC |
| | | AGACTGACCCTGGACAAAATCGCCGTGCTG |
| | | AAAGAGGGCGATCTGTACCCAGACAAAGGC |
| | | GGCGAGAAGTTCATCAGCCTGAGCAAGGAT |
| | | CGGAAGTGCGTGACCACACACGCCGACATC |
| | | AACGCCGCTCAGAACCTGCAGAAGCGGTTC |
| | | TGGACAAGAACCCACGGCTTCTACAAGGTG |
| | | TACTGCAAGGCCTACCAGGTGGACGGCCAG |
| | | ACCGTGTACATCCCTGAGAGCAAGGACCAG |
| | | AAGCAGAAGATCATCGAAGAGTTCGGCGAG |
| | | GGCTACTTCATTCTGAAGGACGGGGTGTAC |
| | | GAATGGGTCAACGCCGGCAAGGGAGGCTCT |
| | | GGAGGAAGCTCCGAAGTCGAGTTTTCCCAT |
| | | GAGTACTGGATGAGACACGCATTGACTCTC |
| | | GCAAAGAGGGCTCGAGATGAACGCGAGGTG |
| | | CCCGTGGGGGCAGTACTCGTGCTCAACAAT |
| | | CGCGTAATCGGCGAAGGTTGGAATAGGGCA |
| | | ATCGGACTCCACGACCCCACTGCACATGCG |
| | | GAAATCATGGCCCTTCGACAGGGAGGGCTT |
| | | GTGATGCAGAATTATCGACTTTATGATGCG |
| | | ACGCTGTACGTCACGTTTGAACCTTGCGTA |
| | | ATGTGCGCGGGAGCTATGATTCACTCCCGC |
| | | ATTGGACGAGTTGTATTCGGTGTTCGCAAC |
| | | GCCAAGACGGGTGCCGCAGGTTCACTGATG |
| | | GACGTGCTGCATCATCCAGGCATGAACCAC |
| | | CGGGTAGAAATCACAGAAGGCATATTGGCG |
| | | GACGAATGTGCGGCGCTGTTGTGTCGTTTT |
| | | TTTCGCATGCCCAGGCGGGTCTTTAACGCC |
| | | CAGAAAAAAGCACAATCCTCTACTGACGGC |
| | | TCTTCTGGATCTGAAACACCTGGCACAAGC |
| | | GAGAGCGCCACCCCTGAGAGCTCTGGCCTG |
| | | AAAATCAAGAAGGGCAGCTCCAAGCAGAGC |
| | | AGCAGCGAGCTGGTGGATAGCGACATCCTG |
| | | AAAGACAGCTTCGACCTGGCCTCCGAGCTG |
| | | AAAGGCGAAAAGCTGATGCTGTACAGGGAC |
| | | CCCAGCGGCAATGTGTTCCCCAGCGACAAA |
| | | TGGATGGCCGCTGGCGTGTTCTTCGGAAAG |
| | | CTGGAACGCATCCTGATCAGCAAGCTGACC |
| | | AACCAGTACTCCATCAGCACCATCGAGGAC |
| | | GACAGCAGCAAGCAGTCTATGAAAAGGCCG |
| | | GCGGCCACGAAAAAGGCCGGCCAGGCAAAA |
| | | AAGAAAAAGGGATCCTACCCATACGATGTT |
| | | CCAGATTACGCTTATCCCTACGACGTGCCT |
| | | GATTATGCATACCCATATGATGTCCCCGAC |
| | | TATGCCTAA |
| BhCas12b | 283 | MAPKKKRKVGIHGVPAAATRSFILKIEPNE |
| GGSGGS- | | EVKKGLWKTHEVLNHGIAYYMNILKLIRQE |
| ABE8- | | AIYEHHEQDPKNPKKVSKAEIQAELWDFVL |
| Xten20 | | KMQKCNSFTHEVDKDEVFNILRELYEELVP |
| at K1019 | | SSVEKKGEANQLSNKFLYPLVDPNSQSGKG |
| polypeptide | | TASSGRKPRWYNLKIAGDPSWEEEKKKWEE |
| | | DKKKDPLAKILGKLAEYGLIPLFIPYTDSN |
| | | EPIVKEIKWMEKSRNQSVRRLDKDMFIQAL |
| | | ERFLSWESWNLKVKEEYEKVEKEYKTLEER |
| | | IKEDIQALKALEQYEKERQEQLLRDTLNTN |
| | | EYRLSKRGLRGWREIIQKWLKMDENEPSEK |
| | | YLEVFKDYQRKHPREAGDYSVYEFLSKKEN |
| | | HFIWRNHPEYPYLYATFCEIDKKKKDAKQQ |
| | | ATFTLADPINHPLWVRFEERSGSNLNKYRI |
| | | LTEQLHTEKLKKKLTVQLDRLIYPTESGGW |
| | | EEKGKVDIVLLPSRQFYNQIFLDIEEKGKH |
| | | AFTYKDESIKFPLKGTLGGARVQFDRDHLR |
| | | RYPHKVESGNVGRIYFNMTVNIEPTESPVS |
| | | KSLKIHRDDFPKVVNFKPKELTEWIKDSKG |
| | | KKLKSGIESLEIGLRVMSIDLGQRQAAAAS |
| | | IFEVVDQKPDIEGKLFFPIKGTELYAVHRA |
| | | SFNIKLPGETLVKSREVLRKAREDNLKLMN |
| | | QKLNFLRNVLHFQQFEDITEREKRVTKWIS |
| | | RQENSDVPLVYQDELIQIRELMYKPYKDWV |
| | | AFLKQLHKRLEVEIGKEVKHWRKSLSDGRK |
| | | GLYGISLKNIDEIDRTRKFLLRWSLRPTEP |
| | | GEVRRLEPGQRFAIDQLNHLNALKEDRLKK |
| | | MANTIIMHALGYCYDVRKKKWQAKNPACQI |
| | | ILFEDLSNYNPYEERSRFENSKLMKWSRRE |
| | | IPRQVALQGEIYGLQVGEVGAQFSSRFHAK |
| | | TGSPGIRCSWTKEKLQDNRFFKNLQREGRL |
| | | TLDKIAVLKEGDLYPDKGGEKFISLSKDRK |
| | | CVTTHADINAAQNLQKRFWTRTHGFYKVYC |
| | | KAYQVDGQTVYIPESKDQKQKIIEEFGEGY |
| | | FILKDGVYEWVNAGKGGSGGSSEVEFSHEY |
| | | WMRHALTLAKRARDE |
| | | REVPVGAVLVLNNRVTGEGWNRATGLHDPT |
| | | AHAETMALRQGGLVMQNYRLYDATLYVTFE |
| | | PCVMCAGAMTHSRTGRWFGVRNAKTGAAGS |
| | | LMDVLHHPGMNHRVETTEGTLADECAALLC |
| | | RFFRMPRRVFNAQKKAQSSTDGSSGSETPG |
| | | TSESATPESSGLKTKKGSSKQSSSELVDSD |
| | | TLKDSFDLASELKGEKLMLYRDPSGNVFPS |
| | | DKWMAAGVFFGKLERTLTSKLTNQYSTSTT |
| | | EDDSSKQSMKRPAATKKAGQAKKKKGSYPY |
| | | DVPDYAYPYDVPDYAYPYDVPDYA |
| tr\|A5H718\| | 41 | MTDAEYVRTHEKLDTYTFKKQFFNNKKSVS |
| A5H718 | | HRCYVLFELKRRGERRACFWGYAVNKPQSG |
| _PETMA | | TERGTHAETFSTRKVEEYLRDNPGQFTTNW |
| Cytosine | | YSSWSPCADCAEKTLEWYNQELRGNGHTLK |
| deaminase | | TWACKLYYEKNARNQTGLWNLRDNGVGLNV |
| OS = | | MVSEHYQCCRKTFTQSSHNQLNENRWLEKT |
| Petromyzon | | LKRAEKRRSELSTMTQVKTLHTTKSPAV |
| marinus | | |
| OX = 7757 | | |
| PE = 2 | | |
| SV = 1 | | |
| amino | | |
| acid | | |
| sequence; | | |
| PmCDA1 | | |
| amino | | |
| acid | | |
| sequence | | |
| EF094822.1 | 42 | TGACACGACACAGCCGTGTATATGAGGAAG |
| Petromyzon | | GGTAGCTGGATGGGGGGGGGGGGAATACGT |
| marinus | | TCAGAGAGGACATTAGCGAGCGTCTTGTTG |
| isolate | | GTGGCCTTGAGTCTAGACACCTGCAGACAT |
| PmCDA.21 | | GACCGACGCTGAGTACGTGAGAATCCATGA |
| cytosine | | GAAGTTGGACATCTACACGTTTAAGAAACA |
| deaminase | | GTTTTTCAACAACAAAAAATCCGTGTCGCA |
| mRNA, | | TAGATGCTACGTTCTCTTTGAATTAAAACG |
| complete cds; | | ACGGGGTGAACGTAGAGCGTGTTTTTGGGG |
| PmCDA1 | | CTATGCTGTGAATAAACCACAGAGCGGGAC |
| amino | | AGAACGTGGAATTCACGCCGAAATCTTTAG |
| acid | | CATTAGAAAAGTCGAAGAATACCTGCGCGA |
| sequence | | CAACCCCGGACAATTCACGATAAATTGGTA |
| | | CTCATCCTGGAGTCCTTGTGCAGATTGCGC |
| | | TGAAAAGATCTTAGAATGGTATAACCAGGA |
| | | GCTGCGGGGGAACGGCCACACTTTGAAAAT |
| | | CTGGGCTTGCAAACTCTATTACGAGAAAAA |
| | | TGCGAGGAATCAAATTGGGCTGTGGAACCT |
| | | CAGAGATAACGGGGTTGGGTTGAATGTAAT |
| | | GGTAAGTGAACACTACCAATGTTGCAGGAA |
| | | AATATTCATCCAATCGTCGCACAATCAATT |
| | | GAATGAGAATAGATGGCTTGAGAAGACTTT |
| | | GAAGCGAGCTGAAAAACGACGGAGCGAGTT |
| | | GTCCATTATGATTCAGGTAAAAATACTCCA |
| | | CACCACTAAGAGTCCTGCTGTTTAAGAGGC |
| | | TATGCGGATGGTTTTC |
| tr\|Q6QJ80\| | 43 | MDSLLMNRRKFLYQFKNVRWAKGRRETYLC |
| Q6QJ80_ | | YWKRRDSATSFSLDFGYLRNKNGCHVELLF |
| HUMAN | | LRYTSDWDLDPGRCYRVTWFTSWSPCYDCA |
| Activation- | | RHVADFLRGNPNLSLRTFTARLYFCEDRKA |
| induced | | EPEGLRRLHRAGVQTATMTFKAPV |
| cytidine | | |
| deaminase | | |
| OS = Homo | | |
| sapiens | | |
| OX = 9606 | | |
| GN = AICDA | | |
| PE = 2 | | |
| SV = 1; | | |
| AID amino | | |
| acid | | |
| sequence | | |
| NG_011588.1: | 44 | AGAGAACCATCATTAATTGAAGTGAGATTT |
| 5001 | | TTCTGGCCTGAGACTTGCAGGGAGGCAAGA |
| -15681 Homo | | AGACACTCTGGACACCACTATGGACAGGTA |
| sapiens | | AAGAGGCAGTCTTCTCGTGGGTGATTGCAC |
| activation | | TGGCCTTCCTCTCAGAGCAAATCTGAGTAA |
| induced | | TGAGACTGGTAGCTATCCCTTTCTCTCATG |
| cytidine | | TAACTGTCTGACTGATAAGATCAGCTTGAT |
| deaminase | | CAATATGCATATATATTTTTTGATCTGTCT |
| (AICDA), | | CCTTTTCTTCTATTCAGATCTTATACGCTG |
| RefSeqGene | | TCAGCCCAATTCTTTCTGTTTCAGACTTCT |
| (LRG_17) on | | CTTGATTTCCCTCTTTTTCATGTGGCAAAA |
| chromosome | | GAAGTAGTGCGTACAATGTACTGATTCGTC |
| 12; nucleic | | CTGAGATTTGTACCATGGTTGAAACTAATT |
| acid | | TATGGTAATAATATTAACATAGCAAATCTT |
| sequence | | TAGAGACTCAAATCATGAAAAGGTAATAGC |
| of the | | AGTACTGTACTAAAAACGGTAGTGCTAATT |
| CDS of | | TTCGTAATAATTTTGTAAATATTCAACAGT |
| human AID | | AAAACAACTTGAAGACACACTTTCCTAGGG |
| | | AGGCGTTACTGAAATAATTTAGCTATAGTA |
| | | AGAAAATTTGTAATTTTAGAAATGCCAAGC |
| | | ATTCTAAATTAATTGCTTGAAAGTCACTAT |
| | | GATTGTGTCCATTATAAGGAGACAAATTCA |
| | | TTCAAGCAAGTTATTTAATGTTAAAGGCCC |
| | | AATTGTTAGGCAGTTAATGGCACTTTTACT |
| | | ATTAACTAATCTTTCCATTTGTTCAGACGT |
| | | AGCTTAACTTACCTCTTAGGTGTGAATTTG |
| | | GTTAAGGTCCTCATAATGTCTTTATGTGCA |
| | | GTTTTTGATAGGTTATTGTCATAGAACTTA |
| | | TTCTATTCCTACATTTATGATTACTATGGA |
| | | TGTATGAGAATAACACCTAATCCTTATACT |
| | | TTACCTCAATTTAACTCCTTTATAAAGAAC |
| | | TTACATTACAGAATAAAGATTTTTTAAAAA |
| | | TATATTTTTTTGTAGAGACAGGGTCTTAGC |
| | | CCAGCCGAGGCTGGTCTCTAAGTCCTGGCC |
| | | CAAGCGATCCTCCTGCCTGGGCCTCCTAAA |
| | | GTGCTGGAATTATAGACATGAGCCATCACA |
| | | TCCAATATACAGAATAAAGATTTTTAATGG |
| | | AGGATTTAATGTTCTTCAGAAAATTTTCTT |
| | | GAGGTCAGACAATGTCAAATGTCTCCTCAG |
| | | TTTACACTGAGATTTTGAAAACAAGTCTGA |
| | | GCTATAGGTCCTTGTGAAGGGTCCATTGGA |
| | | AATACTTGTTCAAAGTAAAATGGAAAGCAA |
| | | AGGTAAAATCAGCAGTTGAAATTCAGAGAA |
| | | AGACAGAAAAGGAGAAAAGATGAAATTCAA |
| | | CAGGACAGAAGGGAAATATATTATCATTAA |
| | | GGAGGACAGTATCTGTAGAGCTCATTAGTG |
| | | ATGGCAAAATGACTTGGTCAGGATTATTTT |
| | | TAACCCGCTTGTTTCTGGTTTGCACGGCTG |
| | | GGGATGCAGCTAGGGTTCTGCCTCAGGGAG |
| | | CACAGCTGTCCAGAGCAGCTGTCAGCCTGC |
| | | AAGCCTGAAACACTCCCTCGGTAAAGTCCT |
| | | TCCTACTCAGGACAGAAATGACGAGAACAG |
| | | GGAGCTGGAAACAGGCCCCTAACCAGAGAA |
| | | GGGAAGTAATGGATCAACAAAGTTAACTAG |
| | | CAGGTCAGGATCACGCAATTCATTTCACTC |
| | | TGACTGGTAACATGTGACAGAAACAGTGTA |
| | | GGCTTATTGTATTTTCATGTAGAGTAGGAC |
| | | CCAAAAATCCACCCAAAGTCCTTTATCTAT |
| | | GCCACATCCTTCTTATCTATACTTCCAGGA |
| | | CACTTTTTCTTCCTTATGATAAGGCTCTCT |
| | | CTCTCTCCACACACACACACACACACACAC |
| | | ACACACACACACACACACACACAAACACAC |
| | | ACCCCGCCAACCAAGGTGCATGTAAAAAGA |
| | | TGTAGATTCCTCTGCCTTTCTCATCTACAC |
| | | AGCCCAGGAGGGTAAGTTAATATAAGAGGG |
| | | ATTTATTGGTAAGAGATGATGCTTAATCTG |
| | | TTTAACACTGGGCCTCAAAGAGAGAATTTC |
| | | TTTTCTTCTGTACTTATTAAGCACCTATTA |
| | | TGTGTTGAGCTTATATATACAAAGGGTTAT |
| | | TATATGCTAATATAGTAATAGTAATGGTGG |
| | | TTGGTACTATGGTAATTACCATAAAAATTA |
| | | TTATCCTTTTAAAATAAAGCTAATTATTAT |
| | | TGGATCTTTTTTAGTATTCATTTTATGTTT |
| | | TTTATGTTTTTGATTTTTTAAAAGACAATC |
| | | TCACCCTGTTACCCAGGCTGGAGTGCAGTG |
| | | GTGCAATCATAGCTTTCTGCAGTCTTGAAC |
| | | TCCTGGGCTCAAGCAATCCTCCTGCCTTGG |
| | | CCTCCCAAAGTGTTGGGATACAGTCATGAG |
| | | CCACTGCATCTGGCCTAGGATCCATTTAGA |
| | | TTAAAATATGCATTTTAAATTTTAAAATAA |
| | | TATGGCTAATTTTTACCTTATGTAATGTGT |
| | | ATACTGGCAATAAATCTAGTTTGCTGCCTA |
| | | AAGTTTAAAGTGCTTTCCAGTAAGCTTCAT |
| | | GTACGTGAGGGGAGACATTTAAAGTGAAAC |
| | | AGACAGCCAGGTGTGGTGGCTCACGCCTGT |
| | | AATCCCAGCACTCTGGGAGGCTGAGGTGGG |
| | | TGGATCGCTTGAGCCCTGGAGTTCAAGACC |
| | | AGCCTGAGCAACATGGCAAAACGCTGTTTC |
| | | TATAACAAAAATTAGCCGGGCATGGTGGCA |
| | | TGTGCCTGTGGTCCCAGCTACTAGGGGGCT |
| | | GAGGCAGGAGAATCGTTGGAGCCCAGGAGG |
| | | TCAAGGCTGCACTGAGCAGTGCTTGCGCCA |
| | | CTGCACTCCAGCCTGGGTGACAGGACCAGA |
| | | CCTTGCCTCAAAAAAATAAGAAGAAAAATT |
| | | AAAAATAAATGGAAACAACTACAAAGAGCT |
| | | GTTGTCCTAGATGAGCTACTTAGTTAGGCT |
| | | GATATTTTGGTATTTAACTTTTAAAGTCAG |
| | | GGTCTGTCACCTGCACTACATTATTAAAAT |
| | | ATCAATTCTCAATGTATATCCACACAAAGA |
| | | CTGGTACGTGAATGTTCATAGTACCTTTAT |
| | | TCACAAAACCCCAAAGTAGAGACTATCCAA |
| | | ATATCCATCAACAAGTGAACAAATAAACAA |
| | | AATGTGCTATATCCATGCAATGGAATACCA |
| | | CCCTGCAGTACAAAGAAGCTACTTGGGGAT |
| | | GAATCCCAAAGTCATGACGCTAAATGAAAG |
| | | AGTCAGACATGAAGGAGGAGATAATGTATG |
| | | CCATACGAAATTCTAGAAAATGAAAGTAAC |
| | | TTATAGTTACAGAAAGCAAATCAGGGCAGG |
| | | CATAGAGGCTCACACCTGTAATCCCAGCAC |
| | | TTTGAGAGGCCACGTGGGAAGATTGCTAGA |
| | | ACTCAGGAGTTCAAGACCAGCCTGGGCAAC |
| | | ACAGTGAAACTCCATTCTCCACAAAAATGG |
| | | GAAAAAAAGAAAGCAAATCAGTGGTTGTCC |
| | | TGTGGGGAGGGGAAGGACTGCAAAGAGGGA |
| | | AGAAGCTCTGGTGGGGTGAGGGTGGTGATT |
| | | CAGGTTCTGTATCCTGACTGTGGTAGCAGT |
| | | TTGGGGTGTTTACATCCAAAAATATTCGTA |
| | | GAATTATGCATCTTAAATGGGTGGAGTTTA |
| | | CTGTATGTAAATTATACCTCAATGTAAGAA |
| | | AAAATAATGTGTAAGAAAACTTTCAATTCT |
| | | CTTGCCAGCAAACGTTATTCAAATTCCTGA |
| | | GCCCTTTACTTCGCAAATTCTCTGCACTTC |
| | | TGCCCCGTACCATTAGGTGACAGCACTAGC |
| | | TCCACAAATTGGATAAATGCATTTCTGGAA |
| | | AAGACTAGGGACAAAATCCAGGCATCACTT |
| | | GTGCTTTCATATCAACCATGCTGTACAGCT |
| | | TGTGTTGCTGTCTGCAGCTGCAATGGGGAC |
| | | TCTTGATTTCTTTAAGGAAACTTGGGTTAC |
| | | CAGAGTATTTCCACAAATGCTATTCAAATT |
| | | AGTGCTTATGATATGCAAGACACTGTGCTA |
| | | GGAGCCAGAAAACAAAGAGGAGGAGAAATC |
| | | AGTCATTATGTGGGAACAACATAGCAAGAT |
| | | ATTTAGATCATTTTGACTAGTTAAAAAAGC |
| | | AGCAGAGTACAAAATCACACATGCAATCAG |
| | | TATAATCCAAATCATGTAAATATGTGCCTG |
| | | TAGAAAGACTAGAGGAATAAACACAAGAAT |
| | | CTTAACAGTCATTGTCATTAGACACTAAGT |
| | | CTAATTATTATTATTAGACACTATGATATT |
| | | TGAGATTTAAAAAATCTTTAATATTTTAAA |
| | | ATTTAGAGCTCTTCTATTTTTCCATAGTAT |
| | | TCAAGTTTGACAATGATCAAGTATTACTCT |
| | | TTCTTTTTTTTTTTTTTTTTTTTTTTTTGA |
| | | GATGGAGTTTTGGTCTTGTTGCCCATGCTG |
| | | GAGTGGAATGGCATGACCATAGCTCACTGC |
| | | AACCTCCACCTCCTGGGTTCAAGCAAAGCT |
| | | GTCGCCTCAGCCTCCCGGGTAGATGGGATT |
| | | ACAGGCGCCCACCACCACACTCGGCTAATG |
| | | TTTGTATTTTTAGTAGAGATGGGGTTTCAC |
| | | CATGTTGGCCAGGCTGGTCTCAAACTCCTG |
| | | ACCTCAGAGGATCCACCTGCCTCAGCCTCC |
| | | CAAAGTGCTGGGATTACAGATGTAGGCCAC |
| | | TGCGCCCGGCCAAGTATTGCTCTTATACAT |
| | | TAAAAAACAGGTGTGAGCCACTGCGCCCAG |
| | | CCAGGTATTGCTCTTATACATTAAAAAATA |
| | | GGCCGGTGCAGTGGCTCACGCCTGTAATCC |
| | | CAGCACTTTGGGAAGCCAAGGCGGGCAGAA |
| | | CACCCGAGGTCAGGAGTCCAAGGCCAGCCT |
| | | GGCCAAGATGGTGAAACCCCGTCTCTATTA |
| | | AAAATACAAACATTACCTGGGCATGATGGT |
| | | GGGCGCCTGTAATCCCAGCTACTCAGGAGG |
| | | CTGAGGCAGGAGGATCCGCGGAGCCTGGCA |
| | | GATCTGCCTGAGCCTGGGAGGTTGAGGCTA |
| | | CAGTAAGCCAAGATCATGCCAGTATACTTC |
| | | AGCCTGGGCGACAAAGTGAGACCGTAACAA |
| | | AAAAAAAAAAATTTAAAAAAAGAAATTTAG |
| | | ATCAAGATCCAACTGTAAAAAGTGGCCTAA |
| | | ACACCACATTAAAGAGTTTGGAGTTTATTC |
| | | TGCAGGCAGAAGAGAACCATCAGGGGGTCT |
| | | TCAGCATGGGAATGGCATGGTGCACCTGGT |
| | | TTTTGTGAGATCATGGTGGTGACAGTGTGG |
| | | GGAATGTTATTTTGGAGGGACTGGAGGCAG |
| | | ACAGACCGGTTAAAAGGCCAGCACAACAGA |
| | | TAAGGAGGAAGAAGATGAGGGCTTGGACCG |
| | | AAGCAGAGAAGAGCAAACAGGGAAGGTACA |
| | | AATTCAAGAAATATTGGGGGGTTTGAATCA |
| | | ACACATTTAGATGATTAATTAAATATGAGG |
| | | ACTGAGGAATAAGAAATGAGTCAAGGATGG |
| | | TTCCAGGCTGCTAGGCTGCTTACCTGAGGT |
| | | GGCAAAGTCGGGAGGAGTGGCAGTTTAGGA |
| | | CAGGGGGCAGTTGAGGAATATTGTTTTGAT |
| | | CATTTTGAGTTTGAGGTACAAGTTGGACAC |
| | | TTAGGTAAAGACTGGAGGGGAAATCTGAAT |
| | | ATACAATTATGGGACTGAGGAACAAGTTTA |
| | | TTTTATTTTTTGTTTCGTTTTCTTGTTGAA |
| | | GAACAAATTTAATTGTAATCCCAAGTCATC |
| | | AGCATCTAGAAGACAGTGGCAGGAGGTGAC |
| | | TGTCTTGTGGGTAAGGGTTTGGGGTCCTTG |
| | | ATGAGTATCTCTCAATTGGCCTTAAATATA |
| | | AGCAGGAAAAGGAGTTTATGATGGATTCCA |
| | | GGCTCAGCAGGGCTCAGGAGGGCTCAGGCA |
| | | GCCAGCAGAGGAAGTCAGAGCATCTTCTTT |
| | | GGTTTAGCCCAAGTAATGACTTCCTTAAAA |
| | | AGCTGAAGGAAAATCCAGAGTGACCAGATT |
| | | ATAAACTGTACTCTTGCATTTTCTCTCCCT |
| | | CCTCTCACCCACAGCCTCTTGATGAACCGG |
| | | AGGAAGTTTCTTTACCAATTCAAAAATGTC |
| | | CGCTGGGCTAAGGGTCGGCGTGAGACCTAC |
| | | CTGTGCTACGTAGTGAAGAGGCGTGACAGT |
| | | GCTACATCCTTTTCACTGGACTTTGGTTAT |
| | | CTTCGCAATAAGGTATCAATTAAAGTCGGC |
| | | TTTGCAAGCAGTTTAATGGTCAACTGTGAG |
| | | TGCTTTTAGAGCCACCTGCTGATGGTATTA |
| | | CTTCCATCCTTTTTTGGCATTTGTGTCTCT |
| | | ATCACATTCCTCAAATCCTTTTTTTTATTT |
| | | CTTTTTCCATGTCCATGCACCCATATTAGA |
| | | CATGGCCCAAAATATGTGATTTAATTCCTC |
| | | CCCAGTAATGCTGGGCACCCTAATACCACT |
| | | CCTTCCTTCAGTGCCAAGAACAACTGCTCC |
| | | CAAACTGTTTACCAGCTTTCCTCAGCATCT |
| | | GAATTGCCTTTGAGATTAATTAAGCTAAAA |
| | | GCATTTTTATATGGGAGAATATTATCAGCT |
| | | TGTCCAAGCAAAAATTTTAAATGTGAAAAA |
| | | CAAATTGTGTCTTAAGCATTTTTGAAAATT |
| | | AAGGAAGAAGAATTTGGGAAAAAATTAACG |
| | | GTGGCTCAATTCTGTCTTCCAAATGATTTC |
| | | TTTTCCCTCCTACTCACATGGGTCGTAGGC |
| | | CAGTGAATACATTCAACATGGTGATCCCCA |
| | | GAAAACTCAGAGAAGCCTCGGCTGATGATT |
| | | AATTAAATTGATCTTTCGGCTACCCGAGAG |
| | | AATTACATTTCCAAGAGACTTCTTCACCAA |
| | | AATCCAGATGGGTTTACATAAACTTCTGCC |
| | | CACGGGTATCTCCTCTCTCCTAACACGCTG |
| | | TGACGTCTGGGCTTGGTGGAATCTCAGGGA |
| | | AGCATCCGTGGGGTGGAAGGTCATCGTCTG |
| | | GCTCGTTGTTTGATGGTTATATTACCATGC |
| | | AATTTTCTTTGCCTACATTTGTATTGAATA |
| | | CATCCCAATCTCCTTCCTATTCGGTGACAT |
| | | GACACATTCTATTTCAGAAGGCTTTGATTT |
| | | TATCAAGCACTTTCATTTACTTCTCATGGC |
| | | AGTGCCTATTACTTCTCTTACAATACCCAT |
| | | CTGTCTGCTTTACCAAAATCTATTTCCCCT |
| | | TTTCAGATCCTCCCAAATGGTCCTCATAAA |
| | | CTGTCCTGCCTCCACCTAGTGGTCCAGGTA |
| | | TATTTCCACAATGTTACATCAACAGGCACT |
| | | TCTAGCCATTTTCCTTCTCAAAAGGTGCAA |
| | | AAAGCAACTTCATAAACACAAATTAAATCT |
| | | TCGGTGAGGTAGTGTGATGCTGCTTCCTCC |
| | | CAACTCAGCGCACTTCGTCTTCCTCATTCC |
| | | ACAAAAACCCATAGCCTTCCTTCACTCTGC |
| | | AGGACTAGTGCTGCCAAGGGTTCAGCTCTA |
| | | CCTACTGGTGTGCTCTTTTGAGCAAGTTGC |
| | | TTAGCCTCTCTGTAACACAAGGACAATAGC |
| | | TGCAAGCATCCCCAAAGATCATTGCAGGAG |
| | | ACAATGACTAAGGCTACCAGAGCCGCAATA |
| | | AAAGTCAGTGAATTTTAGCGTGGTCCTCTC |
| | | TGTCTCTCCAGAACGGCTGCCACGTGGAAT |
| | | TGCTCTTCCTCCGCTACATCTCGGACTGGG |
| | | ACCTAGACCCTGGCCGCTGCTACCGCGTCA |
| | | CCTGGTTCACCTCCTGGAGCCCCTGCTACG |
| | | ACTGTGCCCGACATGTGGCCGACTTTCTGC |
| | | GAGGGAACCCCAACCTCAGTCTGAGGATCT |
| | | TCACCGCGCGCCTCTACTTCTGTGAGGACC |
| | | GCAAGGCTGAGCCCGAGGGGCTGCGGCGGC |
| | | TGCACCGCGCCGGGGTGCAAATAGCCATCA |
| | | TGACCTTCAAAGGTGCGAAAGGGCCTTCCG |
| | | CGCAGGCGCAGTGCAGCAGCCCGCATTCGG |
| | | GATTGCGATGCGGAATGAATGAGTTAGTGG |
| | | GGAAGCTCGAGGGGAAGAAGTGGGCGGGGA |
| | | TTCTGGTTCACCTCTGGAGCCGAAATTAAA |
| | | GATTAGAAGCAGAGAAAAGAGTGAATGGCT |
| | | CAGAGACAAGGCCCCGAGGAAATGAGAAAA |
| | | TGGGGCCAGGGTTGCTTCTTTCCCCTCGAT |
| | | TTGGAACCTGAACTGTCTTCTACCCCCATA |
| | | TCCCCGCCTTTTTTTCCTTTTTTTTTTTTT |
| | | GAAGATTATTTTTACTGCTGGAATACTTTT |
| | | GTAGAAAACCACGAAAGAACTTTCAAAGCC |
| | | TGGGAAGGGCTGCATGAAAATTCAGTTCGT |
| | | CTCTCCAGACAGCTTCGGCGCATCCTTTTG |
| | | GTAAGGGGCTTCCTCGCTTTTTAAATTTTC |
| | | TTTCTTTCTCTACAGTCTTTTTTGGAGTTT |
| | | CGTATATTTCTTATATTTTCTTATTGTTCA |
| | | ATCACTCTCAGTTTTCATCTGATGAAAACT |
| | | TTATTTCTCCTCCACATCAGCTTTTTCTTC |
| | | TGCTGTTTCACCATTCAGAGCCCTCTGCTA |
| | | AGGTTCCTTTTCCCTCCCTTTTCTTTCTTT |
| | | TGTTGTTTCACATCTTTAAATTTCTGTCTC |
| | | TCCCCAGGGTTGCGTTTCCTTCCTGGTCAG |
| | | AATTCTTTTCTCCTTTTTTTTTTTTTTTTT |
| | | TTTTTTTTTTAAACAAACAAACAAAAAACC |
| | | CAAAAAAACTCTTTCCCAATTTACTTTCTT |
| | | CCAACATGTTACAAAGCCATCCACTCAGTT |
| | | TAGAAGACTCTCCGGCCCCACCGACCCCCA |
| | | ACCTCGTTTTGAAGCCATTCACTCAATTTG |
| | | CTTCTCTCTTTCTCTACAGCCCCTGTATGA |
| | | GGTTGATGACTTACGAGACGCATTTCGTAC |
| | | TTTGGGACTTTGATAGCAACTTCCAGGAAT |
| | | GTCACACACGATGAAATATCTCTGCTGAAG |
| | | ACAGTGGATAAAAAACAGTCCTTCAAGTCT |
| | | TCTCTGTTTTTATTCTTCAACTCTCACTTT |
| | | CTTAGAGTTTACAGAAAAAATATTTATATA |
| | | CGACTCTTTAAAAAGATCTATGTCTTGAAA |
| | | ATAGAGAAGGAACACAGGTCTGGCCAGGGA |
| | | CGTGCTGCAATTGGTGCAGTTTTGAATGCA |
| | | ACATTGTCCCCTACTGGGAATAACAGAACT |
| | | GCAGGACCTGGGAGCATCCTAAAGTGTCAA |
| | | CGTFTTTCTATGACTTTTAGGTAGGATGAG |
| | | AGCAGAAGGTAGATCCTAAAAAGCATGGTG |
| | | AGAGGATCAAATGTTTTTATATCAACATCC |
| | | TTTATTATTTGATTCATTTGAGTTAACAGT |
| | | GGTGTTAGTGATAGATTTTTCTATTCTTTT |
| | | CCCTTGACGTTTACTTTCAAGTAACACAAA |
| | | CTCTTCCATCAGGCCATGATCTATAGGACC |
| | | TCCTAATGAGAGTATCTGGGTGATTGTGAC |
| | | CCCAAACCATCTCTCCAAAGCATTAATATC |
| | | CAATCATGCGCTGTATGTTTTAATCAGCAG |
| | | AAGCATGTTTTTATGTTTGTACAAAAGAAG |
| | | ATTGTTATGGGTGGGGATGGAGGTATAGAC |
| | | CATGCATGGTCACCTTCAAGCTACTTTAAT |
| | | AAAGGATCTTAAAATGGGCAGGAGGACTGT |
| | | GAACAAGACACCCTAATAATGGGTTGATGT |
| | | CTGAAGTAGCAAATCTTCTGGAAACGCAAA |
| | | CTCTTTTAAGGAAGTCCCTAATTTAGAAAC |
| | | ACCCACAAACTTCACATATCATAATTAGCA |
| | | AACAATTGGAAGGAAGTTGCTTGAATGTTG |
| | | GGGAGAGGAAAATCTATTGGCTCTCGTGGG |
| | | TCTCTTCATCTCAGAAATGCCAATCAGGTC |
| | | AAGGTTTGCTACATTTTGTATGTGTGTGAT |
| | | GCTTCTCCCAAAGGTATATTAACTATATAA |
| | | GAGAGTTGTGACAAAACAGAATGATAAAGC |
| | | TGCGAACCGTGGCACACGCTCATAGTTCTA |
| | | GCTGCTTGGGAGGTTGAGGAGGGAGGATGG |
| | | CTTGAACACAGGTGTTCAAGGCCAGCCTGG |
| | | GCAACATAACAAGATCCTGTCTCTCAAAAA |
| | | AAAAAAAAAAAAAAAGAAAGAGAGAGGGCC |
| | | GGGCGTGGTGGCTCACGCCTGTAATCCCAG |
| | | CACTTTGGGAGGCCGAGCCGGGCGGATCAC |
| | | CTGTGGTCAGGAGTTTGAGACCAGCCTGGC |
| | | CAACATGGCAAAACCCCGTCTGTACTCAAA |
| | | ATGCAAAAATTAGCCAGGCGTGGTAGCAGG |
| | | CACCTGTAATCCCAGCTACTTGGGAGGCTG |
| | | AGGCAGGAGAATCGCTTGAACCCAGGAGGT |
| | | GGAGGTTGCAGTAAGCTGAGATCGTGCCGT |
| | | TGCACTCCAGCCTGGGCGACAAGAGCAAGA |
| | | CTCTGTCTCAGAAAAAAAAAAAAAAAAGAG |
| | | AGAGAGAGAGAAAGAGAACAATATTTGGGA |
| | | GAGAAGGATGGGGAAGCATTGCAAGGAAAT |
| | | TGTGCTTTATCCAACAAAATGTAAGGAGCC |
| | | AATAAGGGATCCCTATTTGTCTCTTTTGGT |
| | | GTCTATTTGTCCCTAACAACTGTCTTTGAC |
| | | AGTGAGAAAAATATTCAGAATAACCATATC |
| | | CCTGTGCCGTTATTACCTAGCAACCCTTGC |
| | | AATGAAGATGAGCAGATCCACAGGAAAACT |
| | | TGAATGCACAACTGTCTTATTTTAATCTTA |
| | | TTGTACATAAGTTTGTAAAAGAGTTAAAAA |
| | | TTGTTACTTCATGTATTCATTTATATTTTA |
| | | TATTATTTTGCGTCTAATGATTTTTTATTA |
| | | ACATGATTTCCTTTTCTGATATATTGAAAT |
| | | GGAGTCTCAAAGCTTCATAAATTTATAACT |
| | | TTAGAAATGATTCTAATAACAACGTATGTA |
| | | ATTGTAACATTGCAGTAATGGTGCTACGAA |
| | | GCCATTTCTCTTGATTTTTAGTAAACTTTT |
| | | ATGACAGCAAATTTGCTTCTGGCTCACTTT |
| | | CAATCAGTTAAATAAATGATAAATAATTTT |
| | | GGAAGCTGTGAAGATAAAATACCAAATAAA |
| | | ATAATATAAAAGTGATTTATATGAAGTTAA |
| | | AATAAAAAATCAGTATGATGGAATAAACTT |
| | | G |
| Canine | 1374 | MDSLLMKQRKFLYHFKNVRWAKGRHETYLC |
| AID | | YVVKRRDSATSFSLDFGHLRNKSGCHVELL |
| (clAID) | | FLRYISDWDLDPGRCYRVTWFTSWSPCYDC |
| polypeptide | | ARHVADFLRGYPNLSLRIFAARLYFCEDRK |
| sequence | | AEPEGLRRLHRAGVQIAIMTFKDYFYCWNT |
| | | FVENREKTFKAWEGLHENSVRLSRQLRRIL |
| | | LPLYEVDDLRDAFRTLGL |
| Bovine | 1375 | MDSLLKKQRQFLYQFKNVRWAKGRHETYLC |
| AID | | YVVKRRDSPTSFSLDFGHLRNKAGCHVELL |
| (btAID) | | FLRYISDWDLDPGRCYRVTWFTSWSPCYDC |
| polypeptide | | ARHVADFLRGYPNLSLRIFTARLYFCDKER |
| sequence | | KAEPEGLRRLHRAGVQIAIMTFKDYFYCWN |
| | | TFVENHERTFKAWEGLHENSVRLSRQLRRI |
| | | LLPLYEVDDLRDAFRTLGL |
| Rat AID | 1376 | MAVGSKPKAALVGPHWERERIWCFLCSTGL |
| polypeptide | | GTQQTGQTSRWLRPAATQDPVSPPRSLLMK |
| sequence | | QRKFLYHFKNVRWAKGRHETYLCYVVKRRD |
| | | SATSFSLDFGYLRNKSGCHVELLFLRYISD |
| | | WDLDPGRCYRVTWFTSWSPCYDCARHVADF |
| | | LRGNPNLSLRIFTARLTGWGALPAGLMSPA |
| | | RPSDYFYCWNTFVENHERTFKAWEGLHENS |
| | | VRLSRRLRRILLPLYEVDDLRDAFRTLGL |
| Mouse | 1377 | MDSLLMNRRKFLYQFKNVRWAKGRRETYLC |
| (mAID) AID | | YVVKRRDSATSFSLDFGYLRNKNGCHVELL |
| polypeptide | | FLRYISDWDLDPGRCYRVTWFTSWSPCYDC |
| sequence | | ARHVADFLRGNPNLSLRIFTARLYFCEDRK |
| | | AEPEGLRRLHRAGVQIAIMTFKDYFYCWNT |
| | | FVENHERTFKAWEGLHENSVRLSRQLRRIL |
| | | LPLYEVDDLRDAFRTLGL |
| rAPOBEC-1 | 1378 | MSSETGPVAVDPTLRRRIEPHEFEVFFDPR |
| polypeptide | | ELRKETCLLYEINWGGRHSIWRHTSQNTNK |
| sequence | | HVEVNFIEKFTTERYFCPNTRCSITWFLSW |
| | | SPCGECSRAITEFLSRYPHVTLFIYIARLY |
| | | HHADPRNRQGLRDLISSGVTIQIMTEQESG |
| | | YCWRNFVNYSPSNEAHWPRYPHLWVRLYVL |
| | | ELYCIILGLPPCLNILRRKQPQLTFFTIAL |
| | | QSCHYQRLPPHILWATGLK |
| maAPOBEC-1 | 1379 | MSSETGPVVVDPTLRRRIEPHEFDAFFDQG |
| polypeptide | | ELRKETCLLYEIRWGGRHNIWRHTGQNTSR |
| sequence | | HVEINFIEKFTSERYFYPSTRCSIVWFLSW |
| | | SPCGECSKAITEFLSGHPNVTLFIYAARLY |
| | | HHTDQRNRQGLRDLISRGVTIRIMTEQEYC |
| | | YCWRNFVNYPPSNEVYWPRYPNLWMRLYAL |
| | | ELYCIHLGLPPCLKIKRRHQYPLTFFRLNL |
| | | QSCHYQRIPPHILWATGFI |
| ppAPOBEC-1 | 1380 | MTSEKGPSTGDPTLRRRIESWEFDVFYDPR |
| polypeptide | | ELRKETCLLYEIKWGMSRKIWRSSGKNTTN |
| sequence | | HVEVNFIKKFTSERRFHSSISCSITWFLSW |
| | | SPCWECSQAIREFLSQHPGVTLVIYVARLF |
| | | WHMDQRNRQGLRDLVNSGVTIQIMRASEYY |
| | | HCWRNFVNYPPGDEAHWPQYPPLWMMLYAL |
| | | ELHCIILSLPPCLKISRRWQNHLAFFRLHL |
| | | QNCHYQTIPPHILLATGLIHPSVTWR |
| ocAPOBECI | 1381 | MASEKGPSNKDYTLRRRIEPWEFEVFFDPQ |
| polypeptide | | ELRKEACLLYEIKWGASSKTWRSSGKNTTN |
| sequence | | HVEVNFLEKLTSEGRLGPSTCCSITWFLSW |
| | | SPCWECSMAIREFLSQHPGVTLIIFVARLF |
| | | QHMDRRNRQGLKDLVTSGVTVRVMSVSEYC |
| | | YCWENFVNYPPGKAAQWPRYPPRWMLMYAL |
| | | ELYCIILGLPPCLKISRRHQKQLTFFSLTP |
| | | QYCHYKMIPPYILLATGLLQPSVPWR |
| mdAPOBEC-1 | 1382 | MNSKTGPSVGDATLRRRIKPWEFVAFFNPQ |
| polypeptide | | ELRKETCLLYEIKWGNQNIWRHSNQNTSQH |
| sequence | | AEINFMEKFTAERHFNSSVRCSITWFLSWS |
| | | PCWECSKAIRKFLDHYPNVTLAIFISRLYW |
| | | HMDQQHRQGLKELVHSGVTIQIMSYSEYHY |
| | | CWRNFVDYPQGEEDYWPKYPYLWIMLYVLE |
| | | LHCIILGLPPCLKISGSHSNQLALFSLDLQ |
| | | DCHYQKIPYNVLVATGLVQPFVTWR |
| ppAPOBEC-2 | 1383 | MAQKEEAAAATEAASQNGEDLENLDDPEKL |
| polypeptide | | KELIELPPFEIVTGERLPANFFKFQFRNVE |
| sequence | | YSSGRNKTFLCYVVEAQGKGGQVQASRGYL |
| | | EDEHAAAHAEEAFFNTILPAFDPALRYNVT |
| | | WYVSSSPCAACADRIIKTLSKTKNLRLLIL |
| | | VGRLFMWEELEIQDALKKLKEAGCKLRIMK |
| | | PQDFEYVWQNFVEQEEGESKAFQPWEDIQE |
| | | NFLYYEEKLADILK |
| btAPOBEC-2 | 1384 | MAQKEEAAAAAEPASQNGEEVENLEDPEKL |
| polypeptide | | KELIELPPFEIVTGERLPAHYFKFQFRNVE |
| sequence | | YSSGRNKTFLCYVVEAQSKGGQVQASRGYL |
| | | EDEHATNHAEEAFFNSIMPTFDPALRYMVT |
| | | WYVSSSPCAACADRIVKTLNKTKNLRLLIL |
| | | VGRLFMWEEPEIQAALRKLKEAGCRLRIMK |
| | | PQDFEYIWQNFVEQEEGESKAFEPWEDIQE |
| | | NFLYYEEKLADILK |
| mAPOBEC- | 1385 | MQPQRLGPRAGMGPFCLGCSHRKCYSPIRN |
| 3-(1) | | LISQETFKFHFKNLGYAKGRKDTFLCYEVT |
| polypeptide | | RKDCDSPVSLHHGVFKNKDNIHAEICFLYW |
| sequence | | FHDKVLKVLSPREEFKITWYMSWSPCFECA |
| | | EQIVRFLATHHNLSLDIFSSRLYNVQDPET |
| | | QQNLCRLVQEGAQVAAMDLYEFKKCWKKFV |
| | | DNGGRRFRPWKRLLTNFRYQDSKLQEILRP |
| | | CYISVPSSSSSTLSNICLTKGLPETRFWVE |
| | | GRRMDPLSEEEFYSQFYNQRVKHLCYYHRM |
| | | KPYLCYQLEQFNGQAPLKGCLLSEKGKQHA |
| | | EILFLDKIRSMELSQVTITCYLTWSPCPNC |
| | | AWQLAAFKRDRPDLILHIYTSRLYFHWKRP |
| | | FQKGLCSLWQSGILVDVMDLPQFTDCWTNF |
| | | VNPKRPFWPWKGLEIISRRTQRRLRRIKES |
| | | WGLQDLVNDFGNLQLGPPMS |
| APOBEC- | 1386 | MGPFCLGCSHRKCYSPIRNLISQETFKFHF |
| 3-(2) | | KNLGYAKGRKDTFLCYEVTRKDCDSPVSLH |
| (Mouse | | HGVFKNKDNIHAEICFLYWFHDKVLKVLSP |
| APOBEC-3) | | REEFKITWYMSWSPCFECAEQIVRFLATHH |
| polypeptide | | NLSLDIFSSRLYNVQDPETQQNLCRLVQEG |
| sequence | | AQVAAMDLYEFKKCWKKFVDNGGRRFRPWK |
| | | RLLTNFRYQDSKLQEILRPCYIPVPSSSSS |
| | | TLSNICLTKGLPETRFCVEGRRMDPLSEEE |
| | | FYSQFYNQRVKHLCYYHRMKPYLCYQLEQF |
| | | NGQAPLKGCLLSEKGKQHAEILFLDKIRSM |
| | | ELSQVTITCYLTWSPCPNCAWQLAAFKRDR |
| | | PDLILHIYTSRLYFHWKRPFQKGLCSLWQS |
| | | GILVDVMDLPQFTDCWTNFVNPKRPFWPWK |
| | | GLEIISRRTQRRLRRIKESWGLQDLVNDFG |
| | | NLQLGPPMS |
| APOBEC-3 | 1387 | MGPFCLGCSHRKCYSPIRNLISQETFKFHF |
| polypeptide | | KNRLRYAIDRKDTFLCYEVTRKDCDSPVSL |
| sequence | | HHGVFKNKDNIHAEICFLYWFHDKVLKVLS |
| | | PREEFKITWYMSWSPCFECAEQVLRFLATH |
| | | HNLSLDIFSSRLYNIRDPENQQNLCRLVQE |
| | | GAQVAAMDLYEFKKCWKKFVDNGGRRFRPW |
| | | KKLLTNFRYQDSKLQEILRPCYIPVPSSSS |
| | | STLSNICLTKGLPETRFCVERRRVHLLSEE |
| | | EFYSQFYNQRVKHLCYYHGVKPYLCYQLEQ |
| | | FNGQAPLKGCLLSEKGKQHAEILFLDKIRS |
| | | MELSQVIITCYLTWSPCPNCAWQLAAFKRD |
| | | RPDLILHIYTSRLYFHWKRPFQKGLCSLWQ |
| | | SGILVDVMDLPQFTDCWTNFVNPKRPFWPW |
| | | KGLEIISRRTQRRLHRIKESWGLQDLVNDF |
| | | GNLQLGPPMS |
| hAPOBEC-3A | 1388 | MEASPASGPRHLMDPHIFTSNFNNGIGRHK |
| polypeptide | | TYLCYEVERLDNGTSVKMDQHRGFLHNQAK |
| sequence | | NLLCGFYGRHAELRFLDLVPSLQLDPAQIY |
| | | RVTWFISWSPCFSWGCAGEVRAFLQENTHV |
| | | RLRIFAARIYDYDPLYKEALQMLRDAGAQV |
| | | SIMTYDEFKHCWDTFVDHQGCPFQPWDGLD |
| | | EHSQALSGRLRAILQNQGN |
| hAPOBEC-3F | 1389 | MKPHFRNTVERMYRDTFSYNFYNRPILSRR |
| polypeptide | | NTVWLCYEVKTKGPSRPRLDAKIFRGQVYS |
| sequence | | QPEHHAEMCFLSWFCGNQLPAYKCFQITWF |
| | | VSWTPCPDCVAKLAEFLAEHPNVTLTISAA |
| | | RLYYYWERDYRRALCRLSQAGARVKIMDDE |
| | | EFAYCWENFVYSEGQPFMPWYKFDDNYAFL |
| | | HRTLKEILRNPMEAMYPHIFYFHFKNLRKA |
| | | YGRNESWLCFTMEWKHHSPVSWKRGVFRNQ |
| | | VDPETHCHAERCFLSWFCDDILSPNTNYEV |
| | | TWYTSWSPCPECAGEVAEFLARHSNVNLTI |
| | | FTARLYYFWDTDYQEGLRSLSQEGASVEIM |
| | | GYKDFKYCWENFVYNDDEPFKPWKGLKYNF |
| | | LFLDSKLQEILE |
| Rhesus | 1390 | MVEPMDPRTFVSNFNNRPILSGLNTVWLCC |
| macaque | | EVKTKDPSGPPLDAKIFQGKVYSKAKYHPE |
| APOBEC-3G | | MRFLRWFHKWRQLHHDQEYKVTWYVSWSPC |
| polypeptide | | TRCANSVATFLAKDPKVTLTIFVARLYYFW |
| sequence | | KPDYQQALRILCQKRGGPHATMKIMNYNEF |
| | | QDCWNKFVDGRGKPFKPRNNLPKHYTLLQA |
| | | TLGELLRHLMDPGTFTSNFNNKPWVSGQHE |
| | | TYLCYKVERLHNDTWVPLNQHRGFLRNQAP |
| | | NIHGFPKGRHAELCFLDLIPFWKLDGQQYR |
| | | VTCFTSWSPCFSCAQEMAKFISNNEHVSLC |
| | | IFAARIYDDQGRYQEGLRALHRDGAKIAMM |
| | | NYSEFEYCWDTFVDRQGRPFQPWDGLDEHS |
| | | QALSGRLRAI |
| Chimpanzee | 1391 | MKPHFRNPVERMYQDTFSDNFYNRPILSHR |
| APOBEC-3G | | NTVWLCYEVKTKGPSRPPLDAKIFRGQVYS |
| polypeptide | | KLKYHPEMRFFHWFSKWRKLHRDQEYEVTW |
| sequence | | YISWSPCTKCTRDVATFLAEDPKVTLTIFV |
| | | ARLYYFWDPDYQEALRSLCQKRDGPRATMK |
| | | IMNYDEFQHCWSKFVYSQRELFEPWNNLPK |
| | | YYILLHIMLGEILRHSMDPPTFTSNFNNEL |
| | | WVRGRHETYLCYEVERLHNDTWVLLNQRRG |
| | | FLCNQAPHKHGFLEGRHAELCFLDVIPFWK |
| | | LDLHQDYRVTCFTSWSPCFSCAQEMAKFIS |
| | | NNKHVSLCIFAARIYDDQGRCQEGLRTLAK |
| | | AGAKISIMTYSEFKHCWDTFVDHQGCPFQP |
| | | WDGLEEHSQALSGRLRAILQNQGN |
| Green | 1392 | MNPQIRNMVEQMEPDIFVYYFNNRPILSGR |
| monkey | | NTVWLCYEVKTKDPSGPPLDANIFQGKLYP |
| APOBEC-3G | | EAKDHPEMKFLHWFRKWRQLHRDQEYEVTW |
| polypeptide | | YVSWSPCTRCANSVATFLAEDPKVTLTIFV |
| sequence | | ARLYYFWKPDYQQALRILCQERGGPHATMK |
| | | IMNYNEFQHCWNEFVDGQGKPFKPRKNLPK |
| | | HYTLLHATLGELLRHVMDPGTFTSNFNNKP |
| | | WVSGQRETYLCYKVERSHNDTWVLLNQHRG |
| | | FLRNQAPDRHGFPKGRHAELCFLDLIPFWK |
| | | LDDQQYRVTCFTSWSPCFSCAQKMAKFISN |
| | | NKHVSLCIFAARIYDDQGRCQEGLRTLHRD |
| | | GAKIAVMNYSEFEYCWDTFVDRQGRPFQPW |
| | | DGLDEHSQALSGRLRAI |
| Human | 1393 | MKPHFRNTVERMYRDTFSYNFYNRPILSRR |
| APOBEC-3G | | NTVWLCYEVKTKGPSRPPLDAKIFRGQVYS |
| polypeptide | | ELKYHPEMRFFHWFSKWRKLHRDQEYEVTW |
| sequence | | YISWSPCTKCTRDMATFLAEDPKVTLTIFV |
| | | ARLYYFWDPDYQEALRSLCQKRDGPRATMK |
| | | IMNYDEFQHCWSKFVYSQRELFEPWNNLPK |
| | | YYILLHIMLGEILRHSMDPPTFTFNFNNEP |
| | | WVRGRHETYLCYEVERMHNDTVWLLNQRRG |
| | | FLCNQAPHKHGFLEGRHAELCFLDVIPFWK |
| | | LDLDQDYRVTCFTSWSPCFSCAQEMAKFIS |
| | | KNKHVSLCIFTARIYDDQGRCQEGLRTLAE |
| | | AGAKISIMTYSEFKHCWDTFVDHQGCPFQP |
| | | WDGLDEHSQDLSGRLRAILQNQEN |
| Human | 1394 | MNPQIRNPMERMYRDTFYDNFENEPILYGR |
| APOBEC-3B | | SYTWLCYEVKIKRGRSNLLWDTGVFRGQVY |
| polypeptide | | FKPQYHAEMCFLSWFCGNQLPAYKCFQITW |
| sequence | | FVSWTPCPDCVAKLAEFLSEHPNVTLTISA |
| | | ARLYYYWERDYRRALCRLSQAGARVTIMDY |
| | | EEFAYCWENFVYNEGQQFMPWYKFDENYAF |
| | | LHRTLKEILRYLMDPDTFTFNFNNDPLVLR |
| | | RRQTYLCYEVERLDNGTVWLMDQHMGFLCN |
| | | EAKNLLCGFYGRHAELRFLDLVPSLQLDPA |
| | | QIYRVTWFISWSPCFSWGCAGEVRAFLQEN |
| | | THVRLRIFAARIYDYDPLYKEALQMLRDAG |
| | | AQVSIMTYDEFEYCWDTFVYRQGCPFQPWD |
| | | GLEEHSQALSGRLRAILQNQGN |
| Rat | 1395 | MQPQGLGPNAGMGPVCLGCSHRRPYSPIRN |
| APOBEC-3B | | PLKKLYQQTFYFHFKNVRYAWGRKNNFLCY |
| polypeptide | | EVNGMDCALPVPLRQGVFRKQGHIHAELCF |
| sequence | | IYWFHDKVLRVLSPMEEFKVTWYMSWSPCS |
| | | KCAEQVARFLAAHRNLSLAIFSSRLYYYLR |
| | | NPNYQQKLCRLIQEGVHVAAMDLPEFKKCW |
| | | NKFVDNDGQPFRPWMRLRINFSFYDCKLQE |
| | | IFSRMNLLREDVFYLQFNNSHRVKPVQNRY |
| | | YRRKSYLCYQLERANGQEPLKGYLLYKKGE |
| | | QHVEILFLEKMRSMELSQVRITCYLTWSPC |
| | | PNCARQLAAFKKDHPDLILRIYTSRLYFWR |
| | | KKFQKGLCTLWRSGIHVDVMDLPQFADCWT |
| | | NFVNPQRPFRPWNELEKNSWRIQRRLRRIK |
| | | ESWGL |
| Bovine | 1396 | DGWEVAFRSGTVLKAGVLGVSMTEGWAGSG |
| APOBEC- | | HPGQGACVWTPGTRNTMNLLREVLFKQQFG |
| 3B | | NQPRVPAPYYRRKTYLCYQLKQRNDLTLDR |
| polypeptide | | GCFRNKKQRHAERFIDKINSLDLNPSQSYK |
| sequence | | IICYITWSPCPNCANELVNFITRNNHLKLE |
| | | IFASRLYFHWIKSFKMGLQDLQNAGISVAV |
| | | MTHTEFEDCWEQFVDNQSRPFQPWDKLEQY |
| | | SASIRRRLQRILTAPI |
| Chimpanzee | 1397 | MNPQIRNPMEWMYQRTFYYNFENEPILYGR |
| APOBEC-3B | | SYTWLCYEVKIRRGHSNLLWDTGVFRGQMY |
| polypeptide | | SQPEHHAEMCFLSWFCGNQLSAYKCFQITW |
| sequence | | FVSWTPCPDCVAKLAKFLAEHPNVTLTIS |
| | | AARLYYYWERDYRRAL |
| | | CRLSQAGARVKIMDDEEFAYCWENFVYNEG |
| | | QPFMPWYKFDDNYAFLHRTLKEIIRHLMDP |
| | | DTFTFNFNNDPLVLRRHQTYLCYEVERLDN |
| | | GTVWLMDQHMGFLCNEAKNLLCGFYGRHAE |
| | | LRFLDLVPSLQLDPAQIYRVTWFISWSPCF |
| | | SWGCAGQVRAFLQENTHVRLRIFAARIYDY |
| | | DPLYKEALQMLRDAGAQVSIMTYDEFEYCW |
| | | DTFVYRQGCPFQPWDGLEEHSQALSGRLRA |
| | | ILQVRASSLCMVPHRPPPPPQSPGPCLPLC |
| | | SEPPLGSLLPTGRPAPSLPFLLTASFSFPP |
| | | PASLPPLPSLSLSPGHLPVPSFHSLTSCSI |
| | | QPPCSSRIRETEGWASVSKEGRDLG |
| Human | 1398 | MNPQIRNPMKAMYPGTFYFQFKNLWEANDR |
| APOBEC- | | NETWLCFTVEGIKRRSVVSWKTGVFRNQVD |
| 3C | | SETHCHAERCFLSWFCDDILSPNTKYQVTW |
| polypeptide | | YTSWSPCPDCAGEVAEFLARHSNVNLTIFT |
| sequence | | ARLYYFQYPCYQEGLRSLSQEGVAVEIMDY |
| | | EDFKYCWENFVYNDNEPFKPWKGLKTNFRL |
| | | LKRRLRESLQ |
| Gorilla | 1399 | MNPQIRNPMKAMYPGTFYFQFKNLWEANDR |
| APOBEC- | | NETWLCFTVEGIKRRSVVSWKTGVFRNQVD |
| 3C | | SETHCHAERCFLSWECDDILSPNTNYQVTW |
| polypeptide | | YTSWSPCPECAGEVAEFLARHSNVNLTIFT |
| sequence | | ARLYYFQDTDYQEGLRSLSQEGVAVKIMDY |
| | | KDFKYCWENFVYNDDEPFKPWKGLKYNFRF |
| | | LKRRLQEILE |
| Rhesus | 1400 | MDGSPASRPRHLMDPNTFTFNFNNDLSVRG |
| macaque | | RHQTYLCYEVERLDNGTWVPMDERRGFLCN |
| APOBEC-3A | | KAKNVPCGDYGCHVELRFLCEVPSWQLDPA |
| polypeptide | | QTYRVTWFISWSPCFRRGCAGQVRVFLQEN |
| sequence | | KHVRLRIFAARIYDY |
| | | DPLYQEALRTLRDAGAQVSIMTYEEFKHCW |
| | | DTFVDRQGRPFQPWDGLDEHSQALSGRLRA |
| | | ILQNQGN |
| Bovine | 1401 | MDEYTFTENFNNQGWPSKTYLCYEMERLDG |
| APOBEC- | | DATIPLDEYKGFVRNKGLDQPEKPCHAELY |
| 3A | | FLGKIHSWNLDRNQHYRLTCFISWSPCYDC |
| polypeptide | | AQKLTTFLKENHHISLHILASRIYTHNRFG |
| sequence | | CHQSGLCELQAAGARITIMTFEDFKHCWET |
| | | FVDHKGKPFQPWEGLNVKSQALCTELQAIL |
| | | KTQQN |
| Human | 1402 | MALLTAETFRLQFNNKRRLRRPYYPRKALL |
| APOBEC- | | CYQLTPQNGSTPTRGYFENKKKCHAEICFI |
| 3H | | NEIKSMGLDETQCYQVTCYLTWSPCSSCAW |
| polypeptide | | ELVDFIKAHDHLNLGIFASRLYYHWCKPQQ |
| sequence | | KGLRLLCGSQVPVEVMGFPKFADCWENFVD |
| | | HEKPLSFNPYKMLEELDKNSRAIKRRLERI |
| | | KIPGVRAQGRYMDILCDAEV |
| Rhesus | 1403 | MALLTAKTFSLQFNNKRRVNKPYYPRKALL |
| macaque | | CYQLTPQNGSTPTRGHLKNKKKDHAEIRFI |
| APOBEC-3H | | NKIKSMGLDETQCYQVTCYLTWSPCPSCAG |
| polypeptide | | ELVDFIKAHRHLNLRIFASRLYYHWRPNYQ |
| sequence | | EGLLLLCGSQVPVEVMGLPEFTDCWENFVD |
| | | HKEPPSFNPSEKLEELDKNSQAIKRRLERI |
| | | KSRSVDVLENGLRSLQLGPVTPSSSIRNSR |
| Human | 1404 | MNPQIRNPMERMYRDTFYDNFENEPILYGR |
| APOBEC- | | SYTWLCYEVKIKRGRSNLLWDTGVFRGPVL |
| 3D | | PKRQSNHRQEVYFRFENHAEMCFLSWFCGN |
| polypeptide | | RLPANRRFQITWFVSWNPCLPCWKVTKFLA |
| sequence | | EHPNVTLTISAARLYYYRDRDWRWVLLRLH |
| | | KAGARVKIMDYEDFAYCWENFVCNEGQPFM |
| | | PWYKFDDNYASLHRTLKEILRNPMEAMYPH |
| | | IFYFHFKNLLKACGRNESWLCFTMEVTKHH |
| | | SAVFRKRGVFRNQVDPETHCHAERCFLSWF |
| | | CDDILSPNTNYEVTWYTSWSPCPECAGEVA |
| | | EFLARHSNVNLTIFTARLCYFWDTDYQEGL |
| | | CSLSQEGASVKIMGYKDFVSCWKNFVYSDD |
| | | EPFKPWKGLQTNFRLLKRRLREILQ |
| Human | 1405 | MTSEKGPSTGDPTLRRRIEPWEFDVFYDPR |
| APOBEC-1 | | ELRKEACLLYEIKWGMSRKIWRSSGKNTTN |
| polypeptide | | HVEVNFIKKFTSERDFHPSMSCSITWFLSW |
| sequence | | SPCWECSQAIREFLSRHPGVTLVIYVARLF |
| | | WHMDQQNRQGLRDLVNSGVTIQIMRASEYY |
| | | HCWRNFVNYPPGDEAHWPQYPPLWMMLYAL |
| | | ELHCIILSLPPCLKISRRWQNHLTFFRLHL |
| | | QNCHYQTIPPHILLATGLIHPSVAWR |
| Mouse | 1406 | MSSETGPVAVDPTLRRRIEPHEFEVFFDPR |
| APOBEC-1 | | ELRKETCLLYEINWGGRHSVWRHTSQNTSN |
| polypeptide | | HVEVNFLEKFTTERYFRPNTRCSITWFLSW |
| sequence | | SPCGECSRAITEFLSRHPYVTLFIYIARLY |
| | | HHTDQRNRQGLRDLISSGVTIQIMTEQEYC |
| | | YCWRNFVNYPPSNEAYWPRYPHLWVKLYVL |
| | | ELYCIILGLPPCLKILRRKQPQLTFFTITL |
| | | QTCHYQRIPPHLLWATGLK |
| Human | 1407 | MAQKEEAAVATEAASQNGEDLENLDDPEKL |
| APOBEC-2 | | KELIELPPFEIVTGERLPANFFKFQFRNVE |
| polypeptide | | YSSGRNKTFLCYVVEAQGKGGQVQASRGYL |
| sequence | | EDEHAAAHAEEAFFNTILPAFDPALRYNVT |
| | | WYVSSSPCAACADRIIKTLSKTKNLRLLIL |
| | | VGRLFMWEEPEIQAALKKLKEAGCKLRIMK |
| | | PQDFEYVWQNFVEQEEGESKAFQPWEDIQE |
| | | NFLYYEEKLADILK |
| Mouse | 1408 | MAQKEEAAEAAAPASQNGDDLENLEDPEKL |
| APOBEC-2 | | KELIDLPPFEIVTGVRLPVNFFKFQFRNVE |
| polypeptide | | YSSGRNKTFLCYWEVQSKGGQAQATQGYLE |
| sequence | | DEHAGAHAEEAFFNTILPAFDPALKYNVTW |
| | | YVSSSPCAACADRILKTLSKTKNLRLLILV |
| | | SRLFMWEEPEVQAALKKLKEAGCKLRIMKP |
| | | QDFEYIWQNFVEQEEGESKAFEPWEDIQEN |
| | | FLYYEEKLADILK |
| Rat | 1409 | MAQKEEAAEAAAPASQNGDDLENLEDPEKL |
| APOBEC-2 | | KELIDLPPFEIVTGVRLPVNFFKFQFRNVE |
| polypeptide | | YSSGRNKTFLCYWEAQSKGGQVQATQGYLE |
| sequence | | DEHAGAHAEEAFFNTILPAFDPALKYNVTW |
| | | YVSSSPCAACADRILKTLSKTKNLRLLILV |
| | | SRLFMWEEPEVQAALKKLKEAGCKLRIMKP |
| | | QDFEYLWQNFVEQEEGESKAFEPWEDIQEN |
| | | FLYYEEKLADILK |
| Petromyzon | 1410 | MTDAEYVRIHEKLDIYTFKKQFFNNKKSVS |
| marinus | | HRCYVLFELKRRGERRACFWGYAVNKPQSG |
| CDA1 | | TERGIHAEIFSIRKVEEYLRDNPGQFTINW |
| (pmCDAI) | | YSSWSPCADCAEKILEWYNQELRGNGHTLK |
| polypeptide | | IWACKLYYEKNARNQIGLWNLRDNGVGLNV |
| sequence | | MVSEHYQCCRKIFIQSSHNQLNENRWLEKT |
| | | LKRAEKRRSELSFMIQVKILHTTKSPAV |
| Human | 1411 | MKPHFRNTVERMYRDTFSYNFYNRPILSRR |
| APOBEC3G | | NTVWLCYEVKTKGPSRPPLDAKIFRGQVYS |
| D316R | | ELKYHPEMRFFHWFSKWRKLHRDQEYEVT |
| D317R | | WYISWSPCTKCTRDMATFLAEDPKVTLTIF |
| polypeptide | | VARLYYFWDPDYQEALRSLCQKRDGPRATM |
| sequence | | KFNYDEFQHCWSKFVYSQRELFEPWNNLPK |
| | | YYILLHFMLGEILRHSMDPPTFTFNFNNEP |
| | | WVRGRHETYLCYEVERMHNDTWVLLNQRRG |
| | | FLCNQAPHKHGFLEGRHAELCFLDVIPFWK |
| | | LDLDQDYRVTCFTSWSPCFSCAQEMAKFIS |
| | | KKHVSLCIFTARIYRRQGRCQEGLRTLAEA |
| | | GAKISFTYSEFKHCWDTFVDHQGCPFQPWD |
| | | GLDEHSQDLSGRLRAILQNQEN |
| Human | 1412 | MDPPTFTFNFNNEPWWGRHETYLCYEVERM |
| APOBEC3G | | HNDTWVLLNQRRGFLCNQAPHKHGFLEGRH |
| chain | | AELCFLDVIPFWKLDLDQDYRVTCFTSWSP |
| A polypeptide | | CFSCAQEMAKFISKNKHVSLCIFTARIYDD |
| sequence | | QGRCQEGLRTLAEAGAKISFTYSEFKHCWD |
| | | TFVDHQGCPFQPWDGLDEHSQDLSGRLRAI |
| | | LQ |
| Human | 1414 | MDPPTFTFNFNNEPWVRGRHETYLCYEVER |
| APOBEC3G | | MHNDTWVLLNQRRGFLCNQAPHKHGFLEGR |
| chain | | HAELCFLDVIPFWKLDLDQDYRVTCFTSWS |
| A D120R | | PCFSCAQEMAKFISKNKHVSLCIFTARIYR |
| D121R | | RQGRCQEGLRTLAEAGAKISFMTYSEFKHC |
| polypeptide | | WDTFVDHQGCPFQPWDGLDEHSQDLSGRLR |
| sequence | | AILQ |
| hAPOBEC-4 | 1415 | MEPIYEEYLANHGTIVKPYYWLSFSLDCSN |
| polypeptide | | CPYHIRTGEEARVSLTEFCQIFGFPYGTTF |
| sequence | | PQTKHLTFYELKTSSGSLVQKGHASSCTGN |
| | | YIHPESMLFEMNGYLDSAIYNNDSIRHIIL |
| | | YSNNSPCNEANHCCISKMYNFLITYPGITL |
| | | SIYFSQLYHTEMDFPASAWNREALRSLASL |
| | | WPRVVLSPISGGIWHSVLHSFISGVSGSHV |
| | | FQPILTGRALADRHNAYEINAITGVKPYFT |
| | | DVLLQTKRNPNTKAQEALESYPLNNAFPGQ |
| | | FFQMPSGQLQPNLPPDLRAPVVFVLVPLRD |
| | | LPPMHMGQNPNKPRNIVRHLNMPQMSFQET |
| | | KDLGRLPTGRSVEIVEITEQFASSKEADEK |
| | | KKKKGKK |
| rAPOBEC-4 | 1416 | MEPLYEEYLTHSGTIVKPYYWLSVSLNCTN |
| polypeptide | | CPYHIRTGEEARVPYTEFHQTFGFPWSTYP |
| sequence | | QTKHLTFYELRSSSGNLIQKGLASNCTGSH |
| | | THPESMLFERDGYLDSLIFHDSNIRHIILY |
| | | SNNSPCDEANHCCISKMYNFLMNYPEVTLS |
| | | VFFSQLYHTENQFPTSAWNREALRGLASLW |
| | | PQVTLSAISGGIWQSILETFVSGISEGLTA |
| | | VRPFTAGRTLTDRYNAYEINCITEVKPYFT |
| | | DALHSWQKENQDQKVWAASENQPLHNTTPA |
| | | QWQPDMSQDCRTPAVFMLVPYRDLPPIHVN |
| | | PSPQKPRTVVRHLNTLQLSASKVKALRKSP |
| | | SGRPVKKEEARKGSTRSQEANETNKSKWKK |
| | | QTLFIKSNICHLLEREQKKIGILSSWSV |
| rAPOBEC-1 | 1421 | MSSETGPVAVDPTLRRRIEPHEFEVFFDPR |
| (delta | | ELRKETCLLYEINWGGRHSIWRHTSQNTNK |
| 177-186) | | HVEVNFIEKFTTERYFCPNTRCSITWFLSW |
| polypeptide | | SPCGECSRAITEFLSRYPHVTLFIYIARLY |
| sequence | | HHADPRNRQGLRDLISSGVTIQIMTEQESG |
| | | YCWRNFVNYSPSNEAHWPRYPHLWVRGLPP |
| | | CLNILRRKQPQLTFFTIALQSCHYQRLPPH |
| | | ILWATGLK |
| rAPOBEC-1 | 1422 | MSSETGPVAVDPTLRRRIEPHEFEVFFDPR |
| (delta | | ELRKETCLLYEINWGGRHSIWRHTSQNTNK |
| 202-213) | | HVEVNFIEKFTTERYFCPNTRCSITWFLSW |
| polypeptide | | SPCGECSRAITEFLSRYPHVTLFIYIARLY |
| sequence | | HHADPRNRQGLRDLISSGVTIQIMTEQESG |
| | | YCWRNFVNYSPSNEAHWPRYPHLWVRLYVL |
| | | ELYCIILGLPPCLNILRRKQPQHYQRLPPH |
| | | ILWATGLK |
| mouse AID | 1373 | MDSLLMKQKKFLYHFKNVRWAKGRHETYLC |
| (mAPOBEC-4) | | YVVKRRDSATSCSLDFGHLRNKSGCHVELL |
| polypeptide | | FLRYISDWDLDPGRCYRVTWFTSWSPCYDC |
| sequence | | ARHVAEFLRWNPNLSLRIFTARLYFCEDRK |
| | | AEPEGLRRLHRAGVQIGIMTFKDYFYCWNT |
| | | FVENRERTFKAWEGLHENSVRLTRQLRRIL |
| | | LPLYEVDDLRDAFRMLGF |
| pmCDA-1 | 1417 | MAGYECVRVSEKLDFDTFEFQFENLHYATE |
| polypeptide | | RHRTYVIFDVKPQSAGGRSRRLWGYIINNP |
| sequence | | NVCHAELILMSMIDRHLESNPGVYAMTWYM |
| | | SWSPCANCSSKLNPWLKNLLEEQGHTLTMH |
| | | FSRIYDRDREGDHRGLRGLKHVSNSFRMGV |
| | | VGRAEVKECLAEYVEASRRTLTWLDTTESM |
| | | AAKMRRKLFCILVRCAGMRESGIPLHLFTL |
| | | QTPLLSGRVVWWRV |
| pmCDA-2 | 1418 | MELREVVDCALASCVRHEPLSRVAFLRCFA |
| polypeptide | | APSQKPRGTVILFYVEGAGRGVTGGHAVNY |
| sequence | | NKQGTSIHAEVLLLSAVRAALLRRRRCEDG |
| | | EEATRGCTLHCYSTYSPCRDCVEYIQEFGA |
| | | STGVRWIHCCRLYELDVNRRRSEAEGVLRS |
| | | LSRLGRDFRLMGPRDAIALLLGGRLANTAD |
| | | GESGASGNAWVTETNVVEPLVDMTGFGDED |
| | | LHAQVQRNKQIREAY |
| | | ANYASAVSLMLGELHVDPDKFPFLAEFLAQ |
| | | TSVEPSGTPRETRGRPRGASSRGPEIGRQR |
| | | PADFERALGAYGLFLHPRIVSREADREEIK |
| | | RDLIVVMRKHNYQGP |
| pmCDA-5 | 1419 | MAGDENVRVSEKLDFDTFEFQFENLHYATE |
| polypeptide | | RHRTYVIFDVKPQSAGGRSRRLWGYIINNP |
| sequence | | NVCHAELILMSMIDRHLESNPGVYAMTWYM |
| | | SWSPCANCSSKLNPWLKNLLEEQGHTLMMH |
| | | FSRIYDRDREGDHRGLRGLKHVSNSFRMGW |
| | | GRAEVKECLAEYVEASRRTLTWLDTTESMA |
| | | AKMRRKLFCILVRCAGMRESGMPLHLFT |
| yCD | 1420 | MVTGGMASKWDQKGMDIAYEEAALGYKEGG |
| polypeptide | | VPIGGCLINNKDGSVLGRGHNMRFQKGSAT |
| sequence | | LHGEISTLENCGRLEGKVYKDTTLYTTLSP |
| | | CDMCTGAIIMYGIPRCVVGENVNFKSKGEK |
| | | YLQTRGHEVVVVDDERCKKIMKQFIDERPQ |
| | | DWFEDIGE |
| NLS | 84 | KRTADGSEFESPKKKRKV |
| NLS | 85 | KRPAATKKAGQAKKKK |
| NLS | 86 | KKTELQTTNAENKTKKL |
| NLS | 87 | KRGINDRNFWRGENGRKTR |
| NLS | 88 | RKSGKIAAIWKRPRK |
| NLS | 89 | PKKKRKV |
| NLS | 90 | MDSLLMNRRKFLYQFKNVRWAKGRRETYLC |
| WT cas9 | 223 | MDKKYSIGLDIGTNSVGWAVITDEYKVPSK |
| domain | | KFKVLGNTDRHSIKKNLIGALLFDSGETAE |
| | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSPEDNEQKQLFVEQ |
| | | HKHYLDEIIEQISEFSKRVILADANLDKVL |
| | | SAYNKHRDKPIREQAENIIHLFTLTNLGAP |
| | | AAFKYFDTTIDRKRYTSTKEVLDATLIHQS |
| | | ITGLYETRIDLSQLGGD |
| gRNA | 230 | GUUUUAGAGCUAGAAAUAGCAAGUUAAAAU |
| scaffold | | AAGGCUAGUCCGUUAUCAACUUGAAAAAGU |
| nucleotide | | GGGACCGAGUCGGUGCUUUU |
| sequence | | |
| wild type | 231 | ATGGATAAGAAATACTCAATAGGCTTAGAT |
| spCas9 | | ATCGGCACAAATAGCGTCGGATGGGCGGTG |
| poly- | | ATCACTGATGATTATAAGGTTCCGTCTAAA |
| nucleotide | | AAGTTCAAGGTTCTGGGAAATACAGACCGC |
| sequence | | CACAGTATCAAAAAAAATCTTATAGGGGCT |
| | | CTTTTATTTGGCAGTGGAGAGACAGCGGAA |
| | | GCGACTCGTCTCAAACGGACAGCTCGTAGA |
| | | AGGTATACACGTCGGAAGAATCGTATTTGT |
| | | TATCTACAGGAGATTTTTTCAAATGAGATG |
| | | GCGAAAGTAGATGATAGTTTCTTTCATCGA |
| | | CTTGAAGAGTCTTTTTTGGTGGAAGAAGAC |
| | | AAGAAGCATGAACGTCATCCTATTTTTGGA |
| | | AATATAGTAGATGAAGTTGCTTATCATGAG |
| | | AAATATCCAACTATCTATCATCTGCGAAAA |
| | | AAATTGGCAGATTCTACTGATAAAGCGGAT |
| | | TTGCGCTTAATCTATTTGGCCTTAGCGCAT |
| | | ATGATTAAGTTTCGTGGTCATTTTTTGATT |
| | | GAGGGAGATTTAAATCCTGATAATAGTGAT |
| | | GTGGACAAACTATTTATCCAGTTGGTACAA |
| | | ATCTACAATCAATTATTTGAAGAAAACCCT |
| | | ATTAACGCAAGTAGAGTAGATGCTAAAGCG |
| | | ATTCTTTCTGCACGATTGAGTAAATCAAGA |
| | | CGATTAGAAAATCTCATTGCTCAGCTCCCC |
| | | GGTGAGAAGAGAAATGGCTTGTTTGGGAAT |
| | | CTCATTGCTTTGTCATTGGGATTGACCCCT |
| | | AATTTTAAATCAAATTTTGATTTGGCAGAA |
| | | GATGCTAAATTACAGCTTTCAAAAGATACT |
| | | TACGATGATGATTTAGATAATTTATTGGCG |
| | | CAAATTGGAGATCAATATGCTGATTTGTTT |
| | | TTGGCAGCTAAGAATTTATCAGATGCTATT |
| | | TTACTTTCAGATATCCTAAGAGTAAATAGT |
| | | GAAATAACTAAGGCTCCCCTATCAGCTTCA |
| | | ATGATTAAGCGCTACGATGAACATCATCAA |
| | | GACTTGACTCTTTTAAAAGCTTTAGTTCGA |
| | | CAACAACTTCCAGAAAAGTATAAAGAAATC |
| | | TTTTTTGATCAATCAAAAAACGGATATGCA |
| | | GGTTATATTGATGGGGGAGCTAGCCAAGAA |
| | | GAATTTTATAAATTTATCAAACCAATTTTA |
| | | GAAAAAATGGATGGTACTGAGGAATTATTG |
| | | GTGAAACTAAATCGTGAAGATTTGCTGCGC |
| | | AAGCAACGGACCTTTGACAACGGCTCTATT |
| | | CCCCATCAAATTCACTTGGGTGAGCTGCAT |
| | | GCTATTTTGAGAAGACAAGAAGACTTTTAT |
| | | CCATTTTTAAAAGACAATCGTGAGAAGATT |
| | | GAAAAAATCTTGACTTTTCGAATTCCTTAT |
| | | TATGTTGGTCCATTGGCGCGTGGCAATAGT |
| | | CGTTTTGCATGGATGACTCGGAAGTCTGAA |
| | | GAAACAATTACCCCATGGAATTTTGAAGAA |
| | | GTTGTCGATAAAGGTGCTTCAGCTCAATCA |
| | | TTTATTGAACGCATGACAAACTTTGATAAA |
| | | AATCTTCCAAATGAAAAAGTACTACCAAAA |
| | | CATAGTTTGCTTTATGAGTATTTTACGGTT |
| | | TATAACGAATTGACAAAGGTCAAATATGTT |
| | | ACTGAGGGAATGCGAAAACCAGCATTTCTT |
| | | TCAGGTGAACAGAAGAAAGCCATTGTTGAT |
| | | TTACTCTTCAAAACAAATCGAAAAGTAACC |
| | | GTTAAGCAATTAAAAGAAGATTATTTCAAA |
| | | AAAATAGAATGTTTTGATAGTGTTGAAATT |
| | | TCAGGAGTTGAAGATAGATTTAATGCTTCA |
| | | TTAGGCGCCTACCATGATTTGCTAAAAATT |
| | | ATTAAAGATAAAGATTTTTTGGATAATGAA |
| | | GAAAATGAAGATATCTTAGAGGATATTGTT |
| | | TTAACATTGACCTTATTTGAAGATAGGGGG |
| | | ATGATTGAGGAAAGACTTAAAACATATGCT |
| | | CACCTCTTTGATGATAAGGTGATGAAACAG |
| | | CTTAAACGTCGCCGTTATACTGGTTGGGGA |
| | | CGTTTGTCTCGAAAATTGATTAATGGTATT |
| | | AGGGATAAGCAATCTGGCAAAACAATATTA |
| | | GATTTTTTGAAATCAGATGGTTTTGCCAAT |
| | | CGCAATTTTATGCAGCTGATCCATGATGAT |
| | | AGTTTGACATTTAAAGAAGATATTCAAAAA |
| | | GCACAGGTGTCTGGACAAGGCCATAGTTTA |
| | | CATGAACAGATTGCTAACTTAGCTGGCAGT |
| | | CCTGCTATTAAAAAAGGTATTTTACAGACT |
| | | GTAAAAATTGTTGATGAACTGGTCAAAGTA |
| | | ATGGGGCATAAGCCAGAAAATATCGTTATT |
| | | GAAATGGCACGTGAAAATCAGACAACTCAA |
| | | AAGGGCCAGAAAAATTCGCGAGAGCGTATG |
| | | AAACGAATCGAAGAAGGTATCAAAGAATTA |
| | | GGAAGTCAGATTCTTAAAGAGCATCCTGTT |
| | | GAAAATACTCAATTGCAAAATGAAAAGCTC |
| | | TATCTCTATTATCTACAAAATGGAAGAGAC |
| | | ATGTATGTGGACCAAGAATTAGATATTAAT |
| | | CGTTTAAGTGATTATGATGTCGATCACATT |
| | | GTTCCACAAAGTTTCATTAAAGACGATTCA |
| | | ATAGACAATAAGGTACTAACGCGTTCTGAT |
| | | AAAAATCGTGGTAAATCGGATAACGTTCCA |
| | | AGTGAAGAAGTAGTCAAAAAGATGAAAAAC |
| | | TATTGGAGACAACTTCTAAACGCCAAGTTA |
| | | ATCACTCAACGTAAGTTTGATAATTTAACG |
| | | AAAGCTGAACGTGGAGGTTTGAGTGAACTT |
| | | GATAAAGCTGGTTTTATCAAACGCCAATTG |
| | | GTTGAAACTCGCCAAATCACTAAGCATGTG |
| | | GCACAAATTTTGGATAGTCGCATGAATACT |
| | | AAATACGATGAAAATGATAAACTTATTCGA |
| | | GAGGTTAAAGTGATTACCTTAAAATCTAAA |
| | | TTAGTTTCTGACTTCCGAAAAGATTTCCAA |
| | | TTCTATAAAGTACGTGAGATTAACAATTAC |
| | | CATCATGCCCATGATGCGTATCTAAATGCC |
| | | GTCGTTGGAACTGCTTTGATTAAGAAATAT |
| | | CCAAAACTTGAATCGGAGTTTGTCTATGGT |
| | | GATTATAAAGTTTATGATGTTCGTAAAATG |
| | | ATTGCTAAGTCTGAGCAAGAAATAGGCAAA |
| | | GCAACCGCAAAATATTTCTTTTACTCTAAT |
| | | ATCATGAACTTCTTCAAAACAGAAATTACA |
| | | CTTGCAAATGGAGAGATTCGCAAACGCCCT |
| | | CTAATCGAAACTAATGGGGAAACTGGAGAA |
| | | ATTGTCTGGGATAAAGGGCGAGATTTTGCC |
| | | ACAGTGCGCAAAGTATTGTCCATGCCCCAA |
| | | GTCAATATTGTCAAGAAAACAGAAGTACAG |
| | | ACAGGCGGATTCTCCAAGGAGTCAATTTTA |
| | | CCAAAAAGAAATTCGGACAAGCTTATTGCT |
| | | CGTAAAAAAGACTGGGATCCAAAAAAATAT |
| | | GGTGGTTTTGATAGTCCAACGGTAGCTTAT |
| | | TCAGTCCTAGTGGTTGCTAAGGTGGAAAAA |
| | | GGGAAATCGAAGAAGTTAAAATCCGTTAAA |
| | | GAGTTACTAGGGATCACAATTATGGAAAGA |
| | | AGTTCCTTTGAAAAAAATCCGATTGACTTT |
| | | TTAGAAGCTAAAGGATATAAGGAAGTTAAA |
| | | AAAGACTTAATCATTAAACTACCTAAATAT |
| | | AGTCTTTTTGAGTTAGAAAACGGTCGTAAA |
| | | CGGATGCTGGCTAGTGCCGGAGAATTACAA |
| | | AAAGGAAATGAGCTGGCTCTGCCAAGCAAA |
| | | TATGTGAATTTTTTATATTTAGCTAGTCAT |
| | | TATGAAAAGTTGAAGGGTAGTCCAGAAGAT |
| | | AACGAACAAAAACAATTGTTTGTGGAGCAG |
| | | CATAAGCATTATTTAGATGAGATTATTGAG |
| | | CAAATCAGTGAATTTTCTAAGCGTGTTATT |
| | | TTAGCAGATGCCAATTTAGATAAAGTTCTT |
| | | AGTGCATATAACAAACATAGAGACAAACCA |
| | | ATACGTGAACAAGCAGAAAATATTATTCAT |
| | | TTATTTACGTTGACGAATCTTGGAGCTCCC |
| | | GCTGCTTTTAAATATTTTGATACAACAATT |
| | | GATCGTAAACGATATACGTCTACAAAAGAA |
| | | GTTTTAGATGCCACTCTTATCCATCAATCC |
| | | ATCACTGGTCTTTATGAAACACGCATTGAT |
| | | TTGAGTCAGCTAGGAGGTGACTGA |
| spCas9 | 232 | MDKKYSIGLDIGTNSVGWAVITDDYKVPSK |
| polypeptide | | KFKVLGNTDRHSIKKNLIGALLFGSGETAE |
| sequence | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLADSTDKAD |
| | | LRLIYLALAHMIKFRGHFLIEGDLNPDNSD |
| | | VDKLFIQLVQIYNQLFEENPINASRVDAKA |
| | | ILSARLSKSRRLENLIAQLPGEKRNGLFGN |
| | | LIALSLGLTPNFKSNFDLAEDAKLQLSKDT |
| | | YDDDLDNLLAQIGDQYADLFLAAKNLSDAI |
| | | LLSDILRVNSEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGAYHDLLKI |
| | | IKDKDFLDNEENEDILEDIVLTLTLFEDRG |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGHSL |
| | | HEQIANLAGSPAIKKGILQTVKIVDELVKV |
| | | MGHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFIKDDSIDNKVLTRSDKNRGKSDNVP |
| | | SEEWKKMKNYWRQLLNAKLITQRKFDNLTK |
| | | AERGGLSELDKAGFIKRQLVETRQITKHVA |
| | | QILDSRMNTKYDENDKLIREVKVITLKSKL |
| | | VSDFRKDFQFYKVREINNYHHAHDAYLNAV |
| | | VGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFDSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| wild- | 235 | ATGGATAAAAAGTATTCTATTGGTTTAGAC |
| type | | ATCGGCACTAATTCCGTTGGATGGGCTGTC |
| Cas9 | | ATAACCGATGAATACAAAGTACCTTCAAAG |
| poly- | | AAATTTAAGGTGTTGGGGAACACAGACCGT |
| nucleotide | | CATTCGATTAAAAAGAATCTTATCGGTGCC |
| sequence | | CTCCTATTCGATAGTGGCGAAACGGCAGAG |
| | | GCGACTCGCCTGAAACGAACCGCTCGGAGA |
| | | AGGTATACACGTCGCAAGAACCGAATATGT |
| | | TACTTACAAGAAATTTTTAGCAATGAGATG |
| | | GCCAAAGTTGACGATTCTTTCTTTCACCGT |
| | | TTGGAAGAGTCCTTCCTTGTCGAAGAGGAC |
| | | AAGAAACATGAACGGCACCCCATCTTTGGA |
| | | AACATAGTAGATGAGGTGGCATATCATGAA |
| | | AAGTACCCAACGATTTATCACCTCAGAAAA |
| | | AAGCTAGTTGACTCAACTGATAAAGCGGAC |
| | | CTGAGGTTAATCTACTTGGCTCTTGCCCAT |
| | | ATGATAAAGTTCCGTGGGCACTTTCTCATT |
| | | GAGGGTGATCTAAATCCGGACAACTCGGAT |
| | | GTCGACAAACTGTTCATCCAGTTAGTACAA |
| | | ACCTATAATCAGTTGTTTGAAGAGAACCCT |
| | | ATAAATGCAAGTGGCGTGGATGCGAAGGCT |
| | | ATTCTTAGCGCCCGCCTCTCTAAATCCCGA |
| | | CGGCTAGAAAACCTGATCGCACAATTACCC |
| | | GGAGAGAAGAAAAATGGGTTGTTCGGTAAC |
| | | CTTATAGCGCTCTCACTAGGCCTGACACCA |
| | | AATTTTAAGTCGAACTTCGACTTAGCTGAA |
| | | GATGCCAAATTGCAGCTTAGTAAGGACACG |
| | | TACGATGACGATCTCGACAATCTACTGGCA |
| | | CAAATTGGAGATCAGTATGCGGACTTATTT |
| | | TTGGCTGCCAAAAACCTTAGCGATGCAATC |
| | | CTCCTATCTGACATACTGAGAGTTAATACT |
| | | GAGATTACCAAGGCGCCGTTATCCGCTTCA |
| | | ATGATCAAAAGGTACGATGAACATCACCAA |
| | | GACTTGACACTTCTCAAGGCCCTAGTCCGT |
| | | CAGCAACTGCCTGAGAAATATAAGGAAATA |
| | | TTCTTTGATCAGTCGAAAAACGGGTACGCA |
| | | GGTTATATTGACGGCGGAGCGAGTCAAGAG |
| | | GAATTCTACAAGTTTATCAAACCCATATTA |
| | | GAGAAGATGGATGGGACGGAAGAGTTGCTT |
| | | GTAAAACTCAATCGCGAAGATCTACTGCGA |
| | | AAGCAGCGGACTTTCGACAACGGTAGCATT |
| | | CCACATCAAATCCACTTAGGCGAATTGCAT |
| | | GCTATACTTAGAAGGCAGGAGGATTTTTAT |
| | | CCGTTCCTCAAAGACAATCGTGAAAAGATT |
| | | GAGAAAATCCTAACCTTTCGCATACCTTAC |
| | | TATGTGGGACCCCTGGCCCGAGGGAACTCT |
| | | CGGTTCGCATGGATGACAAGAAAGTCCGAA |
| | | GAAACGATTACTCCATGGAATTTTGAGGAA |
| | | GTTGTCGATAAAGGTGCGTCAGCTCAATCG |
| | | TTCATCGAGAGGATGACCAACTTTGACAAG |
| | | AATTTACCGAACGAAAAAGTATTGCCTAAG |
| | | CACAGTTTACTTTACGAGTATTTCACAGTG |
| | | TACAATGAACTCACGAAAGTTAAGTATGTC |
| | | ACTGAGGGCATGCGTAAACCCGCCTTTCTA |
| | | AGCGGAGAACAGAAGAAAGCAATAGTAGAT |
| | | CTGTTATTCAAGACCAACCGCAAAGTGACA |
| | | GTTAAGCAATTGAAAGAGGACTACTTTAAG |
| | | AAAATTGAATGCTTCGATTCTGTCGAGATC |
| | | TCCGGGGTAGAAGATCGATTTAATGCGTCA |
| | | CTTGGTACGTATCATGACCTCCTAAAGATA |
| | | ATTAAAGATAAGGACTTCCTGGATAACGAA |
| | | GAGAATGAAGATATCTTAGAAGATATAGTG |
| | | TTGACTCTTACCCTCTTTGAAGATCGGGAA |
| | | ATGATTGAGGAAAGACTAAAAACATACGCT |
| | | CACCTGTTCGACGATAAGGTTATGAAACAG |
| | | TTAAAGAGGCGTCGCTATACGGGCTGGGGA |
| | | CGATTGTCGCGGAAACTTATCAACGGGATA |
| | | AGAGACAAGCAAAGTGGTAAAACTATTCTC |
| | | GATTTTCTAAAGAGCGACGGCTTCGCCAAT |
| | | AGGAACTTTATGCAGCTGATCCATGATGAC |
| | | TCTTTAACCTTCAAAGAGGATATACAAAAG |
| | | GCACAGGTTTCCGGACAAGGGGACTCATTG |
| | | CACGAACATATTGCGAATCTTGCTGGTTCG |
| | | CCAGCCATCAAAAAGGGCATACTCCAGACA |
| | | GTCAAAGTAGTGGATGAGCTAGTTAAGGTC |
| | | ATGGGACGTCACAAACCGGAAAACATTGTA |
| | | ATCGAGATGGCACGCGAAAATCAAACGACT |
| | | CAGAAGGGGCAAAAAAACAGTCGAGAGCGG |
| | | ATGAAGAGAATAGAAGAGGGTATTAAAGAA |
| | | CTGGGCAGCCAGATCTTAAAGGAGCATCCT |
| | | GTGGAAAATACCCAATTGCAGAACGAGAAA |
| | | CTTTACCTCTATTACCTACAAAATGGAAGG |
| | | GACATGTATGTTGATCAGGAACTGGACATA |
| | | AACCGTTTATCTGATTACGACGTCGATCAC |
| | | ATTGTACCCCAATCCTTTTTGAAGGACGAT |
| | | TCAATCGACAATAAAGTGCTTACACGCTCG |
| | | GATAAGAACCGAGGGAAAAGTGACAATGTT |
| | | CCAAGCGAGGAAGTCGTAAAGAAAATGAAG |
| | | AACTATTGGCGGCAGCTCCTAAATGCGAAA |
| | | CTGATAACGCAAAGAAAGTTCGATAACTTA |
| | | ACTAAAGCTGAGAGGGGTGGCTTGTCTGAA |
| | | CTTGACAAGGCCGGATTTATTAAACGTCAG |
| | | CTCGTGGAAACCCGCCAAATCACAAAGCAT |
| | | GTTGCACAGATACTAGATTCCCGAATGAAT |
| | | ACGAAATACGACGAGAACGATAAGCTGATT |
| | | CGGGAAGTCAAAGTAATCACTTTAAAGTCA |
| | | AAATTGGTGTCGGACTTCAGAAAGGATTTT |
| | | CAATTCTATAAAGTTAGGGAGATAAATAAC |
| | | TACCACCATGCGCACGACGCTTATCTTAAT |
| | | GCCGTCGTAGGGACCGCACTCATTAAGAAA |
| | | TACCCGAAGCTAGAAAGTGAGTTTGTGTAT |
| | | GGTGATTACAAAGTTTATGACGTCCGTAAG |
| | | ATGATCGCGAAAAGCGAACAGGAGATAGGC |
| | | AAGGCTACAGCCAAATACTTCTTTTATTCT |
| | | AACATTATGAATTTCTTTAAGACGGAAATC |
| | | ACTCTGGCAAACGGAGAGATACGCAAACGA |
| | | CCTTTAATTGAAACCAATGGGGAGACAGGT |
| | | GAAATCGTATGGGATAAGGGCCGGGACTTC |
| | | GCGACGGTGAGAAAAGTTTTGTCCATGCCC |
| | | CAAGTCAACATAGTAAAGAAAACTGAGGTG |
| | | CAGACCGGAGGGTTTTCAAAGGAATCGATT |
| | | CTTCCAAAAAGGAATAGTGATAAGCTCATC |
| | | GCTCGTAAAAAGGACTGGGACCCGAAAAAG |
| | | TACGGTGGCTTCGATAGCCCTACAGTTGCC |
| | | TATTCTGTCCTAGTAGTGGCAAAAGTTGAG |
| | | AAGGGAAAATCCAAGAAACTGAAGTCAGTC |
| | | AAAGAATTATTGGGGATAACGATTATGGAG |
| | | CGCTCGTCTTTTGAAAAGAACCCCATCGAC |
| | | TTCCTTGAGGCGAAAGGTTACAAGGAAGTA |
| | | AAAAAGGATCTCATAATTAAACTACCAAAG |
| | | TATAGTCTGTTTGAGTTAGAAAATGGCCGA |
| | | AAACGGATGTTGGCTAGCGCCGGAGAGCTT |
| | | CAAAAGGGGAACGAACTCGCACTACCGTCT |
| | | AAATACGTGAATTTCCTGTATTTAGCGTCC |
| | | CATTACGAGAAGTTGAAAGGTTCACCTGAA |
| | | GATAACGAACAGAAGCAACTTTTTGTTGAG |
| | | CAGCACAAACATTATCTCGACGAAATCATA |
| | | GAGCAAATTTCGGAATTCAGTAAGAGAGTC |
| | | ATCCTAGCTGATGCCAATCTGGACAAAGTA |
| | | TTAAGCGCATACAACAAGCACAGGGATAAA |
| | | CCCATACGTGAGCAGGCGGAAAATATTATC |
| | | CATTTGTTTACTCTTACCAACCTCGGCGCT |
| | | CCAGCCGCATTCAAGTATTTTGACACAACG |
| | | ATAGATCGCAAACGATACACTTCTACCAAG |
| | | GAGGTGCTAGACGCGACACTGATTCACCAA |
| | | TCCATCACGGGATTATATGAAACTCGGATA |
| | | GATTTGTCACAGCTTGGGGGTGACGGATCC |
| | | CCCAAGAAGAAGAGGAAAGTCTCGAGCGAC |
| | | TACAAAGACCATGACGGTGATTATAAAGAT |
| | | CATGACATCGATTACAAGGATGACGATGAC |
| | | AAGGCTGCAGGA |
| wild-type | 236 | MDKKYSIGLAIGTNSVGWAVITDEYKVPSK |
| Cas9 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFDSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| Cas9 from | 237 | ATGGATAAGAAATACTCAATAGGCTTAGAT |
| Strepto- | | ATCGGCACAAATAGCGTCGGATGGGCGGTG |
| coccus | | ATCACTGATGAATATAAGGTTCCGTCTAAA |
| pyogenes | | AAGTTCAAGGTTCTGGGAAATACAGACCGC |
| (NCBI | | CACAGTATCAAAAAAAATCTTATAGGGGCT |
| Ref. Seq.: | | CTTTTATTTGACAGTGGAGAGACAGCGGAA |
| NC_002737.2) | | GCGACTCGTCTCAAACGGACAGCTCGTAGA |
| poly- | | AGGTATACACGTCGGAAGAATCGTATTTGT |
| nucleotide | | TATCTACAGGAGATTTTTTCAAATGAGATG |
| sequence | | GCGAAAGTAGATGATAGTTTCTTTCATCGA |
| | | CTTGAAGAGTCTTTTTTGGTGGAAGAAGAC |
| | | AAGAAGCATGAACGTCATCCTATTTTTGGA |
| | | AATATAGTAGATGAAGTTGCTTATCATGAG |
| | | AAATATCCAACTATCTATCATCTGCGAAAA |
| | | AAATTGGTAGATTCTACTGATAAAGCGGAT |
| | | TTGCGCTTAATCTATTTGGCCTTAGCGCAT |
| | | ATGATTAAGTTTCGTGGTCATTTTTTGATT |
| | | GAGGGAGATTTAAATCCTGATAATAGTGAT |
| | | GTGGACAAACTATTTATCCAGTTGGTACAA |
| | | ACCTACAATCAATTATTTGAAGAAAACCCT |
| | | ATTAACGCAAGTGGAGTAGATGCTAAAGCG |
| | | ATTCTTTCTGCACGATTGAGTAAATCAAGA |
| | | CGATTAGAAAATCTCATTGCTCAGCTCCCC |
| | | GGTGAGAAGAAAAATGGCTTATTTGGGAAT |
| | | CTCATTGCTTTGTCATTGGGTTTGACCCCT |
| | | AATTTTAAATCAAATTTTGATTTGGCAGAA |
| | | GATGCTAAATTACAGCTTTCAAAAGATACT |
| | | TACGATGATGATTTAGATAATTTATTGGCG |
| | | CAAATTGGAGATCAATATGCTGATTTGTTT |
| | | TTGGCAGCTAAGAATTTATCAGATGCTATT |
| | | TTACTTTCAGATATCCTAAGAGTAAATACT |
| | | GAAATAACTAAGGCTCCCCTATCAGCTTCA |
| | | ATGATTAAACGCTACGATGAACATCATCAA |
| | | GACTTGACTCTTTTAAAAGCTTTAGTTCGA |
| | | CAACAACTTCCAGAAAAGTATAAAGAAATC |
| | | TTTTTTGATCAATCAAAAAACGGATATGCA |
| | | GGTTATATTGATGGGGGAGCTAGCCAAGAA |
| | | GAATTTTATAAATTTATCAAACCAATTTTA |
| | | GAAAAAATGGATGGTACTGAGGAATTATTG |
| | | GTGAAACTAAATCGTGAAGATTTGCTGCGC |
| | | AAGCAACGGACCTTTGACAACGGCTCTATT |
| | | CCCCATCAAATTCACTTGGGTGAGCTGCAT |
| | | GCTATRTGAGAAGACAAGAAGACTTTTATC |
| | | CATTTTTAAAAGACAATCGTGAGAAGATTG |
| | | AAAAAATCTTGACTTTTCGAATTCCTTATT |
| | | ATGTTGGTCCATTGGCGCGTGGCAATAGTC |
| | | GTTTTGCATGGATGACTCGGAAGTCTGAAG |
| | | AAACAATTACCCCATGGAATTTTGAAGAAG |
| | | TTGTCGATAAAGGTGCTTCAGCTCAATCAT |
| | | TTATTGAACGCATGACAAACTTTGATAAAA |
| | | ATCTTCCAAATGAAAAAGTACTACCAAAAC |
| | | ATAGTTTGCTTTATGAGTATTTTACGGTTT |
| | | ATAACGAATTGACAAAGGTCAAATATGTTA |
| | | CTGAAGGAATGCGAAAACCAGCATTTCTTT |
| | | CAGGTGAACAGAAGAAAGCCATTGTTGATT |
| | | TACTCTTCAAAACAAATCGAAAAGTAACCG |
| | | TTAAGCAATTAAAAGAAGATTATTTCAAAA |
| | | AAATAGAATGTTTTGATAGTGTTGAAATTT |
| | | CAGGAGTTGAAGATAGATTTAATGCTTCAT |
| | | TAGGTACCTACCATGATTTGCTAAAAATTA |
| | | TTAAAGATAAAGATTTTTTGGATAATGAAG |
| | | AAAATGAAGATATCTTAGAGGATATTGTTT |
| | | TAACATTGACCTTATTTGAAGATAGGGAGA |
| | | TGATTGAGGAAAGACTTAAAACATATGCTC |
| | | ACCTCTTTGATGATAAGGTGATGAAACAGC |
| | | TTAAACGTCGCCGTTATACTGGTTGGGGAC |
| | | GTTTGTCTCGAAAATTGATTAATGGTATTA |
| | | GGGATAAGCAATCTGGCAAAACAATATTAG |
| | | ATTTTTTGAAATCAGATGGTTTTGCCAATC |
| | | GCAATTTTATGCAGCTGATCCATGATGATA |
| | | GTTTGACATTTAAAGAAGACATTCAAAAAG |
| | | CACAAGTGTCTGGACAAGGCGATAGTTTAC |
| | | ATGAACATATTGCAAATTTAGCTGGTAGCC |
| | | CTGCTATTAAAAAAGGTATTTTACAGACTG |
| | | TAAAAGTTGTTGATGAATTGGTCAAAGTAA |
| | | TGGGGCGGCATAAGCCAGAAAATATCGTTA |
| | | TTGAAATGGCACGTGAAAATCAGACAACTC |
| | | AAAAGGGCCAGAAAAATTCGCGAGAGCGTA |
| | | TGAAACGAATCGAAGAAGGTATCAAAGAAT |
| | | TAGGAAGTCAGATTCTTAAAGAGCATCCTG |
| | | TTGAAAATACTCAATTGCAAAATGAAAAGC |
| | | TCTATCTCTATTATCTCCAAAATGGAAGAG |
| | | ACATGTATGTGGACCAAGAATTAGATATTA |
| | | ATCGTTTAAGTGATTATGATGTCGATCACA |
| | | TTGTTCCACAAAGTTTCCTTAAAGACGATT |
| | | CAATAGACAATAAGGTCTTAACGCGTTCTG |
| | | ATAAAAATCGTGGTAAATCGGATAACGTTC |
| | | CAAGTGAAGAAGTAGTCAAAAAGATGAAAA |
| | | ACTATTGGAGACAACTTCTAAACGCCAAGT |
| | | TAATCACTCAACGTAAGTTTGATAATTTAA |
| | | CGAAAGCTGAACGTGGAGGTTTGAGTGAAC |
| | | TTGATAAAGCTGGTTTTATCAAACGCCAAT |
| | | TGGTTGAAACTCGCCAAATCACTAAGCATG |
| | | TGGCACAAATTTTGGATAGTCGCATGAATA |
| | | CTAAATACGATGAAAATGATAAACTTATTC |
| | | GAGAGGTTAAAGTGATTACCTTAAAATCTA |
| | | AATTAGTTTCTGACTTCCGAAAAGATTTCC |
| | | AATTCTATAAAGTACGTGAGATTAACAATT |
| | | ACCATCATGCCCATGATGCGTATCTAAATG |
| | | CCGTCGTTGGAACTGCTTTGATTAAGAAAT |
| | | ATCCAAAACTTGAATCGGAGTTTGTCTATG |
| | | GTGATTATAAAGTTTATGATGTTCGTAAAA |
| | | TGATTGCTAAGTCTGAGCAAGAAATAGGCA |
| | | AAGCAACCGCAAAATATTTCTTTTACTCTA |
| | | ATATCATGAACTTCTTCAAAACAGAAATTA |
| | | CACTTGCAAATGGAGAGATTCGCAAACGCC |
| | | CTCTAATCGAAACTAATGGGGAAACTGGAG |
| | | AAATTGTCTGGGATAAAGGGCGAGATTTTG |
| | | CCACAGTGCGCAAAGTATTGTCCATGCCCC |
| | | AAGTCAATATTGTCAAGAAAACAGAAGTAC |
| | | AGACAGGCGGATTCTCCAAGGAGTCAATTT |
| | | TACCAAAAAGAAATTCGGACAAGCTTATTG |
| | | CTCGTAAAAAAGACTGGGATCCAAAAAAAT |
| | | ATGGTGGTTTTGATAGTCCAACGGTAGCTT |
| | | ATTCAGTCCTAGTGGTTGCTAAGGTGGAAA |
| | | AAGGGAAATCGAAGAAGTTAAAATCCGTTA |
| | | AAGAGTTACTAGGGATCACAATTATGGAAA |
| | | GAAGTTCCTTTGAAAAAAATCCGATTGACT |
| | | TTTTAGAAGCTAAAGGATATAAGGAAGTTA |
| | | AAAAAGACTTAATCATTAAACTACCTAAAT |
| | | ATAGTCTTTTTGAGTTAGAAAACGGTCGTA |
| | | AACGGATGCTGGCTAGTGCCGGAGAATTAC |
| | | AAAAAGGAAATGAGCTGGCTCTGCCAAGCA |
| | | AATATGTGAATFTTTTATATTTAGCTAGTC |
| | | ATTATGAAAAGTTGAAGGGTAGTCCAGAAG |
| | | ATAACGAACAAAAACAATTGTTTGTGGAGC |
| | | AGCATAAGCATTATTTAGATGAGATTATTG |
| | | AGCAAATCAGTGAATTTTCTAAGCGTGTTA |
| | | TTTTAGCAGATGCCAATTTAGATAAAGTTC |
| | | TTAGTGCATATAACAAACATAGAGACAAAC |
| | | CAATACGTGAACAAGCAGAAAATATTATTC |
| | | ATTTATTTACGTTGACGAATCTTGGAGCTC |
| | | CCGCTGCTTTTAAATATTTTGATACAACAA |
| | | TTGATCGTAAACGATATACGTCTACAAAAG |
| | | AAGTTTTAGATGCCACTCTTATCCATCAAT |
| | | CCATCACTGGTCTTTATGAAACACGCATTG |
| | | ATTTGAGTCAGCTAGGAGGTGACTGA |
| catalyt- | 238 | MDKKYSIGLAIGTNSVGWAVITDEYKVPSK |
| ically | | KFKVLGNTDRHSIKKNLIGALLFDSGETAE |
| inactive | | ATRLKRTARRRYTRRKNRICYLQEIFSNEM |
| Cas9 | | AKVDDSFFHRLEESFLVEEDKKHERHPIFG |
| (dCas9) | | NIVDEVAYHEKYPTIYHLRKKLVDSTDKAD |
| polypeptide | | LRLIYLALAHMIKFRGHFLIEGDLNPDNSD |
| sequence | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDA |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSPEDNEQKQLFVEQ |
| | | HKHYLDEIIEQISEFSKRVILADANLDKVL |
| | | SAYNKHRDKPIREQAENIIHLFTLTNLGAP |
| | | AAFKYFDTTIDRKRYTSTKEVLDATLIHQS |
| | | ITGLYETRIDLSQLGGD |
| tr\|F0NN87\| | 239 | MEVPLYNIFGDNYIIQVATEAENSTIYNNK |
| F0NN87_ | | VEIDDEELRNVLNLAYKIAKNNEDAAAERR |
| SULIHCRISPR- | | GKAKKKKGEEGETTTSNIILPLSGNDKNPW |
| associated | | TETLKCYNFPTTVALSEVFKNFSQVKECEE |
| Casx protein | | VSAPSFVKPEFYEFGRSPGMVERTRRVKLE |
| OS = | | VEPHYLIIAAAGWVLTRLGKAKVSEGDYVG |
| Sulfolobus | | VNVFTPTRGILYSLIQNVNGIVPGIKPETA |
| islandicus | | FGLWIARKWSSVTNPNVSVVRIYTISDAVG |
| (strain | | QNPTTINGGFSIDLTKLLEKRYLLSERLEA |
| HVE10/4) | | IARNALSISSNMRERYIVLANYIYEYLTG |
| GN = | | SKRLEDLLYFANRDLIMNLNSDDGKVRDLK |
| SiH 0402 | | LISAYVNGELIRGEG |
| PE = 4 | | |
| SV = 1); | | |
| CasX | | |
| polypeptide | | |
| sequence | | |
| tr\|F0NH53\| | 240 | MEVPLYNIFGDNYIIQVATEAENSTIYNNK |
| F0NH53_ | | VEIDDEELRNVLNLAYKIAKNNEDAAAERR |
| SULIR | | GKAKKKKGEEGETTTSNIILPLSGNDKNPW |
| CRISPR | | TETLKCYNFPTTVALSEVFKNFSQVKECEE |
| associated | | VSAPSFVKPEFYKFGRSPGMVERTRRVKLE |
| protein, | | VEPHYLIMAAAGWVLTRLGKAKVSEGDYVG |
| Casx OS = | | VNVFTPTRGILYSLIQNVNGIVPGIKPETA |
| Sulfolobus | | FGLWIARKVVSSVTNPNVSVVSIYTISDAV |
| islandicus | | GQNPTTINGGFSIDLTKLLEKRDLLSERLE |
| (strain | | AIARNALSISSNMRERYIVLANYIYEYLTG |
| REY15A) | | SKRLEDLLYFANRDLIMNLNSDDGKVRDLK |
| GN = SiRe | | LISAYVNGELIRGEG |
| 0771 | | |
| PE = 4 | | |
| SV = 1); | | |
| CasX | | |
| polypeptide | | |
| sequence | | |
| CasX | 241 | MEKRINKIRKKLSADNATKPVSRSGPMKTL |
| polypeptide | | LVRVMTDDLKKRLEKRRKKPEVMPQVISNN |
| sequence | | AANNLRMLLDDYTKMKEAILQVYWQEFKDD |
| | | HVGLMCKFAQPASKKIDQNKLKPEMDEKGN |
| | | LTTAGFACSQCGQPLFVYKLEQVSEKGKAY |
| | | TNYFGRCNVAEHEKLILLAQLKPVKDSDEA |
| | | VTYSLGKFGQRALDFYSIHVTKESTHPVKP |
| | | LAQIAGNRYASGPVGKALSDACMGTIASFL |
| | | SKYQDIIIEHQKVVKGNQKRLESLRELAGK |
| | | ENLEYPSVTLPPQPHTKEGVDFAYNEVIAR |
| | | VRMWVNLNLWQKLKLSRDDAKPLLRLKGFP |
| | | SFPVVERRENEVDWWNTINEVKKLIDAKRD |
| | | MGRVFWSGVTAEKRNTILEGYNYLPNENDH |
| | | KKREGSLENPKKPAKRQFGDLLLYLEKKYA |
| | | GDWGKVFDEAWERIDKKIAGLTSHIEREEA |
| | | RNAEDAQSKAVLTDWLRAKASFVLERLKEM |
| | | DEKEFYACEIQLQKWYGDLRGNPFAVEAEN |
| | | RVVDISGFSIGSDGHSIQYRNLLAWKYLEN |
| | | GKREFYLLMNYGKKGRIRFTDGTDIKKSGK |
| | | WQGLLYGGGKAKVIDLTFDPDDEQLIILPL |
| | | AFGTRQGREFIWNDLLSLETGLIKLANGRV |
| | | IEKTIYNKKIGRDEPALFVALTFERREWDP |
| | | SNIKPVNLIGVARGENIPAVIALTDPEGCP |
| | | LPEFKDSSGGPTDILRIGEGYKEKQRAIQA |
| | | AKEVEQRRAGGYSRKFASKSRNLADDMVRN |
| | | SARDLFYHAVTHDAVLVFANLSRGFGRQGK |
| | | RTFMTERQYTKMEDWLTAKLAYEGLTSKTY |
| | | LSKTLAQYTSKTCSNCGFTITYADMDVMLV |
| | | RLKKTSDGWATTLNNKELKAEYQITYYNRY |
| | | KRQTVEKELSAELDRLSEESGNNDISKWTK |
| | | GRRDEALFLLKKRFSHRPVQEQFVCLDCGH |
| | | EVHAAEQAALNIARSWLFLNSNSTEFKSYK |
| | | SGKQPFVGAWQAFYKRRLKEVWKPNA |
| APG80656.1 | 242 | MSKRHPRISGVKGYRLHAQRLEYTGKSGAM |
| CRISPR- | | RTIKYPLYSSPSGGRTVPREIVSAINDDYV |
| associated | | GLYGLSNFDDLYNAEKRNEEKVYSVLDFWY |
| protein | | DCVQYGAVFSYTAPGLLKNVAEVRGGSYEL |
| CasY | | TKTLKGSHLYDELQIDKVIKFLNKKEISRA |
| [uncultured | | NGSLDKLKKDIIDCFKAEYRERHKDQCNKL |
| Parcubacteria | | ADDIKNAKKDAGASLGERQKKLFRDFFGIS |
| group | | EQSENDKPSFTNPLNLTCCLLPFDTVNNNR |
| Bacterium]; | | NRGEVLFNKLKEYAQKLDKNEGSLEMWEYI |
| CasY | | GIGNSGTAFSNFLGEGFLGRLRENKITELK |
| polypeptide | | KAMMDITDAWRGQEQEEELEKRLRILAALT |
| sequence | | IKLREPKFDNHWGGYRSDINGKLSSWLQNY |
| | | INQTVKIKEDLKGHKKDLKKAKEMINRFGE |
| | | SDTKEEAWSSLLESIEKIVPDDSADDEKPD |
| | | IPAIAIYRRFLSDGRLTLNRFVQREDVQEA |
| | | LIKERLEAEKKKKPKKRKKKSDAEDEKETI |
| | | DFKELFPHLAKPLKLVPNFYGDSKRELYKK |
| | | YKNAAIYTDALWKAVEKIYKSAFSSSLKNS |
| | | FFDTDFDKDFFIKRLQKIFSVYRRFNTDKW |
| | | KPIVKNSFAPYCDIVSLAENEVLYKPKQSR |
| | | SRKSAAIDKNRVRLPSTENIAKAGIALARE |
| | | LSVAGFDWKDLLKKEEHEEYIDLIELHKTA |
| | | LALLLAVTETQLDISALDFVENGTVKDFMK |
| | | TRDGNLVLEGRFLEMFSQSIVFSELRGLAG |
| | | LMSRKEFITRSAIQTMNGKQAELLYIPHEF |
| | | QSAKITTPKEMSRAFLDLAPAEFATSLEPE |
| | | SLSEKSLLKLKQMRYYPHYFGYELTRTGQG |
| | | IDGGVAENALRLEKSPVKKREIKCKQYKTL |
| | | GRGQNKIVLYVRSSYYQTQFLEWFLHRPKN |
| | | VQTDVAVSGSFLIDEKKVKTRWNYDALTVA |
| | | LEPVSGSERVFVSQPFTIFPEKSAEEEGQR |
| | | YLGIDIGEYGIAYTALEITGDSAKILDQNF |
| | | ISDPQLKTLREEVKGLKLDQRRGTFAMPST |
| | | KIARIRESLVHSLRNRIHHLALKHKAKIVY |
| | | ELEVSRFEEGKQKIKKVYATLKKADVYSEI |
| | | DADKNLQTTVWGKLAVASEISASYTSQFCG |
| | | ACKKLWRAEMQVDETITTQELIGTVRVIKG |
| | | GTLIDAIKDFMRPPIFDENDTPFPKYRDFC |
| | | DKHHISKKMRGNSCLFICPFCRANADADIQ |
| | | ASQTIALLRYVKEEKKVEDYFERFRKLKNI |
| | | KVLGQMKKI |
| wild type | 246 | MSIYQEFVNKYSLSKTLRFELIPQGKTLEN |
| Cpf1 | | IKARGLILDDEKRAKDYKKAKQIIDKYHQF |
| polypeptide | | FIEEILSSVCISEDLLQNYSDVYFKLKKSD |
| sequence | | DDNLQKDFKSAKDTIKKQISEYIKDSEKFK |
| | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDVVTTMQSFYEQIA |
| | | AFKTVEEKSIKETLSLLFDDLKAQKLDLSK |
| | | IYFKNDKSLTDLSQQVFDDYSVIGTAVLEY |
| | | ITQQIAPKNLDNPSKKEQELIAKKTEKAKY |
| | | LSLETIKLALEEFNKHRDIDKQCRFEEILA |
| | | NFAAIPMIFDEIAQNKDNLAQISIKYQNQG |
| | | KKDLLQASAEDDVKAIKDLLDQTNNLLHKL |
| | | KIFHISQSEDKANILDKDEHFYLVFEECYF |
| | | ELANIVPLYNKIRNYITQKPYSDEKFKLNF |
| | | ENSTLANGWDKNKEPDNTAILFIKDDKYYL |
| | | GVMNKKNNKIFDDKAIKENKGEGYKKIVYK |
| | | LLPGANKMLPKVFFSAKSIKFYNPSEDILR |
| | | IRNHSTHTKNGSPQKGYEKFEFNIEDCRKF |
| | | IDFYKQSISKHPEWKDFGFRFSDTQRYNSI |
| | | DEFYREVENQGYKLTFENISESYIDSVVNQ |
| | | GKLYLFQIYNKDFSAYSKGRPNLHTLYWKA |
| | | LFDERNLQDWYKLNGEAELFYRKQSIPKKI |
| | | THPAKEAIANKNKDNPKKESVFEYDLIKDK |
| | | RFTEDKFFFHCPITINFKSSGANKFNDEIN |
| | | LLLKEKANDVHILSIDRGERHLAYYTLVDG |
| | | KGNIIKQDTFNIIGNDRMKTNYHDKLAAIE |
| | | KDRDSARKDWKKINNIKEMKEGYLSQVVHE |
| | | IAKLVIEYNAIVVFEDLNFGFKRGRFKVEK |
| | | QVYQKLEKMLIEKLNYLVFKDNEFDKTGGV |
| | | LRAYQLTAPFETFKKMGKQTGIIYYVPAGF |
| | | TSKICPVTGFVNQLYPKYESVSKSQEFFSK |
| | | FDKICYNLDKGYFEFSFDYKNFGDKAAKGK |
| | | WTIASFGSRLINFRNSDKNHNWDTREVYPT |
| | | KELEKLLKDYSIEYGHGECIKAAICGESDK |
| | | KFFAKLTSVLNTILQMRNSKTGTELDYLIS |
| | | PVADVNGNFFDSRQAPKNMPQDADANGAYH |
| | | IGLKGLMLLGRIKNNQEGKKLNLVIKNEEY |
| | | FEFVQNRNN |
| Cpf1 D917A | 247 | MSIYQEFVNKYSLSKTLRFELIPQGKTLEN |
| polypeptide | | IKARGLILDDEKRAKDYKKAKQIIDKYHQF |
| sequence | | FIEEILSSVCISEDLLQNYSDVYFKLKKSD |
| | | DDNLQKDFKSAKDTIKKQISEYIKDSEKFK |
| | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDWTTMQSFYEQIAA |
| | | FKTVEEKSIKETLSLLFDDLKAQKLDLSKI |
| | | YFKNDKSLTDLSQQVFDDYSVIGTAVLEYI |
| | | TQQIAPKNLDNPSKKEQELIAKKTEKAKYL |
| | | SLETIKLALEEFNKHRDIDKQCRFEEILAN |
| | | FAAIPMIFDEIAQNKDNLAQISIKYQNQGK |
| | | KDLLQASAEDDVKAIKDLLDQTNNLLHKLK |
| | | IFHISQSEDKANILDKDEHFYLVFEECYFE |
| | | LANIVPLYNKIRNYITQKPYSDEKFKLNFE |
| | | NSTLANGWDKNKEPDNTAILFIKDDKYYLG |
| | | VMNKKNNKIFDDKAIKENKGEGYKKIVYKL |
| | | LPGANKMLPKVFFSAKSIKFYNPSEDILRI |
| | | RNHSTHTKNGSPQKGYEKFEFNIEDCRKFI |
| | | DFYKQSISKHPEWKDFGFRFSDTQRYNSID |
| | | EFYREVENQGYKLTFENISESYIDSVVNQG |
| | | KLYLFQIYNKDFSAYSKGRPNLHTLYWKAL |
| | | FDERNLQDWYKLNGEAELFYRKQSIPKKIT |
| | | HPAKEAIANKNKDNPKKESVFEYDLIKDKR |
| | | FTEDKFFFHCPITINFKSSGANKFNDEINL |
| | | LLKEKANDVHILSIARGERHLAYYTLVDGK |
| | | GNIIKQDTFNIIGNDRMKTNYHDKLAAIEK |
| | | DRDSARKDWKKINNIKEMKEGYLSQVVHEI |
| | | AKLVIEYNAIVVFEDLNFGFKRGRFKVEKQ |
| | | VYQKLEKMLIEKLNYLVFKDNEFDKTGGVL |
| | | RAYQLTAPFETFKKMGKQTGIIYYVPAGFT |
| | | SKICPVTGFVNQLYPKYESVSKSQEFFSKF |
| | | DKICYNLDKGYFEFSFDYKNFGDKAAKGKW |
| | | TIASFGSRLINFRNSDKNHNWDTREVYPTK |
| | | ELEKLLKDYSIEYGHGECIKAAICGESDKK |
| | | FFAKLTSVLNTILQMRNSKTGTELDYLISP |
| | | VADVNGNFFDSRQAPKNMPQDADANGAYHI |
| | | GLKGLMLLGRIKNNQEGKKLNLVIKNEEYF |
| | | EFVQNRNN |
| Cpf1 | 248 | MSIYQEFVNKYSLSKTLRFELIPQGKTLEN |
| E1006A | | IKARGLILDDEKRAKDYKKAKQIIDKYHQF |
| polypeptide | | FIEEILSSVCISEDLLQNYSDVYFKLKKSD |
| sequence | | DDNLQKDFKSAKDTIKKQISEYIKDSEKFK |
| | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDVVTTMQSFYEQIA |
| | | AFKTVEEKSIKETLSLLFDDLKAQKLDLSK |
| | | IYFKNDKSLTDLSQQVFDDYSVIGTAVLEY |
| | | ITQQIAPKNLDNPSKKEQELIAKKTEKAKY |
| | | LSLETIKLALEEFNKHRDIDKQCRFEEILA |
| | | NFAAIPMIFDEIAQNKDNLAQISIKYQNQG |
| | | KKDLLQASAEDDVKAIKDLLDQTNNLLHKL |
| | | KIFHISQSEDKANILDKDEHFYLVFEECYF |
| | | ELANIVPLYNKIRNYITQKPYSDEKFKLNF |
| | | ENSTLANGWDKNKEPDNTAILFIKDDKYYL |
| | | GVMNKKNNKIFDDKAIKENKGEGYKKIVYK |
| | | LLPGANKMLPKVFFSAKSIKFYNPSEDILR |
| | | IRNHSTHTKNGSPQKGYEKFEFNIEDCRKF |
| | | IDFYKQSISKHPEWKDFGFRFSDTQRYNSI |
| | | DEFYREVENQGYKLTFENISESYIDSVVNQ |
| | | GKLYLFQIYNKDFSAYSKGRPNLHTLYWKA |
| | | LFDERNLQDWYKLNGEAELFYRKQSIPKKI |
| | | THPAKEAIANKNKDNPKKESVFEYDLIKDK |
| | | RFTEDKFFFHCPITINFKSSGANKFNDEIN |
| | | LLLKEKANDVHILSIDRGERHLAYYTLVDG |
| | | KGNIIKQDTFNIIGNDRMKTNYHDKLAAIE |
| | | KDRDSARKDWKKINNIKEMKEGYLSQVVHE |
| | | IAKLVIEYNAIVVFADLNFGFKRGRFKVEK |
| | | QVYQKLEKMLIEKLNYLVFKDNEFDKTGGV |
| | | LRAYQLTAPFETFKKMGKQTGIIYYVPAGF |
| | | TSKICPVTGFVNQLYPKYESVSKSQEFFSK |
| | | FDKICYNLDKGYFEFSFDYKNFGDKAAKGK |
| | | WTIASFGSRLINFRNSDKNHNWDTREVYPT |
| | | KELEKLLKDYSIEYGHGECIKAAICGESDK |
| | | KFFAKLTSVLNTILQMRNSKTGTELDYLIS |
| | | PVADVNGNFFDSRQAPKNMPQDADANGAYH |
| | | IGLKGLMLLGRIKNNQEGKKLNLVIKNEEY |
| | | FEFVQNRNN |
| Cpf1 | 249 | MSIYQEFVNKYSLSKTLRFELIPQGKTLEN |
| D1255A | | IKARGLILDDEKRAKDYKKAKQIIDKYHQF |
| polypeptide | | FIEEILSSVCISEDLLQNYSDVYFKLKKSD |
| sequence | | DDNLQKDFKSAKDTIKKQISEYIKDSEKFK |
| | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDWTTMQSFYEQIAA |
| | | FKTVEEKSIKETLSLLFDDLKAQKLDLSKI |
| | | YFKNDKSLTDLSQQVFDDYSVIGTAVLEYI |
| | | TQQIAPKNLDNPSKKEQELIAKKTEKAKYL |
| | | SLETIKLALEEFNKHRDIDKQCRFEEILAN |
| | | FAAIPMIFDEIAQNKDNLAQISIKYQNQGK |
| | | KDLLQASAEDDVKAIKDLLDQTNNLLHKLK |
| | | IFHISQSEDKANILDKDEHFYLVFEECYFE |
| | | LANIVPLYNKIRNYITQKPYSDEKFKLNFE |
| | | NSTLANGWDKNKEPDNTAILFIKDDKYYLG |
| | | VMNKKNNKIFDDKAIKENKGEGYKKIVYKL |
| | | LPGANKMLPKVFFSAKSIKFYNPSEDILRI |
| | | RNHSTHTKNGSPQKGYEKFEFNIEDCRKFI |
| | | DFYKQSISKHPEWKDFGFRFSDTQRYNSID |
| | | EFYREVENQGYKLTFENISESYIDSVVNQG |
| | | KLYLFQIYNKDFSAYSKGRPNLHTLYWKAL |
| | | FDERNLQDWYKLNGEAELFYRKQSIPKKIT |
| | | HPAKEAIANKNKDNPKKESVFEYDLIKDKR |
| | | FTEDKFFFHCPITINFKSSGANKFNDEINL |
| | | LLKEKANDVHILSIDRGERHLAYYTLVDGK |
| | | GNIIKQDTFNIIGNDRMKTNYHDKLAAIEK |
| | | DRDSARKDWKKINNIKEMKEGYLSQWHEIA |
| | | KLVIEYNAIVVFEDLNFGFKRGRFKVEKQV |
| | | YQKLEKMLIEKLNYLVFKDNEFDKTGGVLR |
| | | AYQLTAPFETFKKMGKQTGIIYYVPAGFTS |
| | | KICPVTGFVNQLYPKYESVSKSQEFFSKFD |
| | | KICYNLDKGYFEFSFDYKNFGDKAAKGKWT |
| | | IASFGSRLINFRNSDKNHNWDTREVYPTKE |
| | | LEKLLKDYSIEYGHGECIKAAICGESDKKF |
| | | FAKLTSVLNTILQMRNSKTGTELDYLISPV |
| | | ADVNGNFFDSRQAPKNMPQDAAANGAYHIG |
| | | LKGLMLLGRIKNNQEGKKLNLVIKNEEYFE |
| | | FVQNRNN |
| Cpf1 | 250 | MSIYQEFVNKYSLSKTLRFELIPQGKTLEN |
| D917A/ | | IKARGLILDDEKRAKDYKKAKQIIDKYHQF |
| E1006A | | FIEEILSSVCISEDLLQNYSDVYFKLKKSD |
| polypeptide | | DDNLQKDFKSAKDTIKKQISEYIKDSEKFK |
| sequence | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDVVTTMQSFYEQIA |
| | | AFKTVEEKSIKETLSLLFDDLKAQKLDLSK |
| | | IYFKNDKSLTDLSQQVFDDYSVIGTAVLEY |
| | | ITQQIAPKNLDNPSKKEQELIAKKTEKAKY |
| | | LSLETIKLALEEFNKHRDIDKQCRFEEILA |
| | | NFAAIPMIFDEIAQNKDNLAQISIKYQNQG |
| | | KKDLLQASAEDDVKAIKDLLDQTNNLLHKL |
| | | KIFHISQSEDKANILDKDEHFYLVFEECYF |
| | | ELANIVPLYNKIRNYITQKPYSDEKFKLNF |
| | | ENSTLANGWDKNKEPDNTAILFIKDDKYYL |
| | | GVMNKKNNKIFDDKAIKENKGEGYKKIVYK |
| | | LLPGANKMLPKVFFSAKSIKFYNPSEDILR |
| | | IRNHSTHTKNGSPQKGYEKFEFNIEDCRKF |
| | | IDFYKQSISKHPEWKDFGFRFSDTQRYNSI |
| | | DEFYREVENQGYKLTFENISESYIDSVVNQ |
| | | GKLYLFQIYNKDFSAYSKGRPNLHTLYWKA |
| | | LFDERNLQDVVYKLNGEAELFYRKQSIPKK |
| | | ITHPAKEAIANKNKDNPKKESVFEYDLIKD |
| | | KRFTEDKFFFHCPITINFKSSGANKFNDEI |
| | | NLLLKEKANDVHILSIARGERHLAYYTLVD |
| | | GKGNIIKQDTFNIIGNDRMKTNYHDKLAAI |
| | | EKDRDSARKDWKKINNIKEMKEGYLSQVVH |
| | | EIAKLVIEYNAIVVFADLNFGFKRGRFKVE |
| | | KQVYQKLEKMLIEKLNYLVFKDNEFDKTGG |
| | | VLRAYQLTAPFETFKKMGKQTGIIYYVPAG |
| | | FTSKICPVTGFVNQLYPKYESVSKSQEFFS |
| | | KFDKICYNLDKGYFEFSFDYKNFGDKAAKG |
| | | KWTIASFGSRLINFRNSDKNHNWDTREVYP |
| | | TKELEKLLKDYSIEYGHGECIKAAICGESD |
| | | KKFFAKLTSVLNTILQMRNSKTGTELDYLI |
| | | SPVADVNGNFFDSRQAPKNMPQDADANGAY |
| | | HIGLKGLMLLGRIKNNQEGKKLNLVIKNEE |
| | | YFEFVQNRNN |
| Cpf1 | 251 | MSIYQEFVNKYSLSKTLRFELIPQGKTLEN |
| D917A/ | | IKARGLILDDEKRAKDYKKAKQIIDKYHQF |
| D1255A | | FIEEILSSVCISEDLLQNYSDVYFKLKKSD |
| polypeptide | | DDNLQKDFKSAKDTIKKQISEYIKDSEKFK |
| sequence | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDWTTMQSFYEQIAA |
| | | FKTVEEKSIKETLSLLFDDLKAQKLDLSKI |
| | | YFKNDKSLTDLSQQVFDDYSVIGTAVLEYI |
| | | TQQIAPKNLDNPSKKEQELIAKKTEKAKYL |
| | | SLETIKLALEEFNKHRDIDKQCRFEEILAN |
| | | FAAIPMIFDEIAQNKDNLAQISIKYQNQGK |
| | | KDLLQASAEDDVKAIKDLLDQTNNLLHKLK |
| | | IFHISQSEDKANILDKDEHFYLVFEECYFE |
| | | LANIVPLYNKIRNYITQKPYSDEKFKLNFE |
| | | NSTLANGWDKNKEPDNTAILFIKDDKYYLG |
| | | VMNKKNNKIFDDKAIKENKGEGYKKIVYKL |
| | | LPGANKMLPKVFFSAKSIKFYNPSEDILRI |
| | | RNHSTHTKNGSPQKGYEKFEFNIEDCRKFI |
| | | DFYKQSISKHPEWKDFGFRFSDTQRYNSID |
| | | EFYREVENQGYKLTFENISESYIDSVVNQG |
| | | KLYLFQIYNKDFSAYSKGRPNLHTLYWKAL |
| | | FDERNLQDVVYKLNGEAELFYRKQSIPKKI |
| | | THPAKEAIANKNKDNPKKESVFEYDLIKDK |
| | | RFTEDKFFFHCPITINFKSSGANKFNDEIN |
| | | LLLKEKANDVHILSIARGERHLAYYTLVDG |
| | | KGNIIKQDTFNIIGNDRMKTNYHDKLAAIE |
| | | KDRDSARKDWKKINNIKEMKEGYLSQVVHE |
| | | IAKLVIEYNAIVVFEDLNFGFKRGRFKVEK |
| | | QVYQKLEKMLIEKLNYLVFKDNEFDKTGGV |
| | | LRAYQLTAPFETFKKMGKQTGIIYYVPAGF |
| | | TSKICPVTGFVNQLYPKYESVSKSQEFFSK |
| | | FDKICYNLDKGYFEFSFDYKNFGDKAAKGK |
| | | WTIASFGSRLINFRNSDKNHNWDTREVYPT |
| | | KELEKLLKDYSIEYGHGECIKAAICGESDK |
| | | KFFAKLTSVLNTILQMRNSKTGTELDYLIS |
| | | PVADVNGNFFDSRQAPKNMPQDAAANGAYH |
| | | IGLKGLMLLGRIKNNQEGKKLNLVIKNEEY |
| | | FEFVQNRNN |
| Cpf1 | 252 | MSIYQEFVNKYSLSKTLRFELIPQGKTLEN |
| E1006A/ | | IKARGLILDDEKRAKDYKKAKQIIDKYHQF |
| D1255A | | FIEEILSSVCISEDLLQNYSDVYFKLKKSD |
| polypeptide | | DDNLQKDFKSAKDTIKKQISEYIKDSEKFK |
| sequence | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDVVTTMQSFYEQIA |
| | | AFKTVEEKSIKETLSLLFDDLKAQKLDLSK |
| | | IYFKNDKSLTDLSQQVFDDYSVIGTAVLEY |
| | | ITQQIAPKNLDNPSKKEQELIAKKTEKAKY |
| | | LSLETIKLALEEFNKHRDIDKQCRFEEILA |
| | | NFAAIPMIFDEIAQNKDNLAQISIKYQNQG |
| | | KKDLLQASAEDDVKAIKDLLDQTNNLLHKL |
| | | KIFHISQSEDKANILDKDEHFYLVFEECYF |
| | | ELANIVPLYNKIRNYITQKPYSDEKFKLNF |
| | | ENSTLANGWDKNKEPDNTAILFIKDDKYYL |
| | | GVMNKKNNKIFDDKAIKENKGEGYKKIVYK |
| | | LLPGANKMLPKVFFSAKSIKFYNPSEDILR |
| | | IRNHSTHTKNGSPQKGYEKFEFNIEDCRKF |
| | | IDFYKQSISKHPEWKDFGFRFSDTQRYNSI |
| | | DEFYREVENQGYKLTFENISESYIDSVVNQ |
| | | GKLYLFQIYNKDFSAYSKGRPNLHTLYWKA |
| | | LFDERNLQDVVYKLNGEAELFYRKQSIPKK |
| | | ITHPAKEAIANKNKDNPKKESVFEYDLIKD |
| | | KRFTEDKFFFHCPITINFKSSGANKFNDEI |
| | | NLLLKEKANDVHILSIDRGERHLAYYTLVD |
| | | GKGNIIKQDTFNIIGNDRMKTNYHDKLAAI |
| | | EKDRDSARKDWKKINNIKEMKEGYLSQVVH |
| | | EIAKLVIEYNAIVVFADLNFGFKRGRFKVE |
| | | KQVYQKLEKMLIEKLNYLVFKDNEFDKTGG |
| | | VLRAYQLTAPFETFKKMGKQTGIIYYVPAG |
| | | FTSKICPVTGFVNQLYPKYESVSKSQEFFS |
| | | KFDKICYNLDKGYFEFSFDYKNFGDKAAKG |
| | | KWTIASFGSRLINFRNSDKNHNWDTREVYP |
| | | TKELEKLLKDYSIEYGHGECIKAAICGESD |
| | | KKFFAKLTSVLNTILQMRNSKTGTELDYLI |
| | | SPVADVNGNFFDSRQAPKNMPQDAAANGAY |
| | | HIGLKGLMLLGRIKNNQEGKKLNLVIKNEE |
| | | YFEFVQNRNN |
| Cpf1 | 253 | MSIYQEFVNKYSLSKTLRFELIPQGKTLEN |
| D917A/ | | IKARGLILDDEKRAKDYKKAKQIIDKYHQF |
| E1006A/ | | FIEEILSSVCISEDLLQNYSDVYFKLKKSD |
| D1255A | | DDNLQKDFKSAKDTIKKQISEYIKDSEKFK |
| polypeptide | | NLFNQNLIDAKKGQESDLILWLKQSKDNGI |
| sequence | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDWTTMQSFYEQIAA |
| | | FKTVEEKSIKETLSLLFDDLKAQKLDLSKI |
| | | YFKNDKSLTDLSQQVFDDYSVIGTAVLEYI |
| | | TQQIAPKNLDNPSKKEQELIAKKTEKAKYL |
| | | SLETIKLALEEFNKHRDIDKQCRFEEILAN |
| | | FAAIPMIFDEIAQNKDNLAQISIKYQNQGK |
| | | KDLLQASAEDDVKAIKDLLDQTNNLLHKLK |
| | | IFHISQSEDKANILDKDEHFYLVFEECYFE |
| | | LANIVPLYNKIRNYITQKPYSDEKFKLNFE |
| | | NSTLANGWDKNKEPDNTAILFIKDDKYYLG |
| | | VMNKKNNKIFDDKAIKENKGEGYKKIVYKL |
| | | LPGANKMLPKVFFSAKSIKFYNPSEDILRI |
| | | RNHSTHTKNGSPQKGYEKFEFNIEDCRKFI |
| | | DFYKQSISKHPEWKDFGFRFSDTQRYNSID |
| | | EFYREVENQGYKLTFENISESYIDSVVNQG |
| | | KLYLFQIYNKDFSAYSKGRPNLHTLYWKAL |
| | | FDERNLQDWYKLNGEAELFYRKQSIPKKIT |
| | | HPAKEAIANKNKDNPKKESVFEYDLIKDKR |
| | | FTEDKFFFHCPITINFKSSGANKFNDEINL |
| | | LLKEKANDVHILSIARGERHLAYYTLVDGK |
| | | GNIIKQDTFNIIGNDRMKTNYHDKLAAIEK |
| | | DRDSARKDWKKINNIKEMKEGYLSQVVHEI |
| | | AKLVIEYNAIVVFADLNFGFKRGRFKVEKQ |
| | | VYQKLEKMLIEKLNYLVFKDNEFDKTGGVL |
| | | RAYQLTAPFETFKKMGKQTGIIYYVPAGFT |
| | | SKICPVTGFVNQLYPKYESVSKSQEFFSKF |
| | | DKICYNLDKGYFEFSFDYKNFGDKAAKGKW |
| | | TIASFGSRLINFRNSDKNHNWDTREVYPTK |
| | | ELEKLLKDYSIEYGHGECIKAAICGESDKK |
| | | FFAKLTSVLNTILQMRNSKTGTELDYLISP |
| | | VADVNGNFFDSRQAPKNMPQDAAANGAYHI |
| | | GLKGLMLLGRIKNNQEGKKLNLVIKNEEYF |
| | | EFVQNRNN |
| synthetic | 254 | KRNYILGLDIGITSVGYGIIDYETRDVIDA |
| polypeptide | | GVRLFKEANVENNEGRRSKRGARRLKRRRR |
| | | HRIQRVKKLLFDYNLLTDHSELSGINPYEA |
| | | RVKGLSQKLSEEEFSAALLHLAKRRGVHNV |
| | | NEVEEDTGNELSTKEQISRNSKALEEKYVA |
| | | ELQLERLKKDGEVRGSINRFKTSDYVKEAK |
| | | QLLKVQKAYHQLDQSFIDTYIDLLETRRTY |
| | | YEGPGEGSPFGWKDIKEWYEMLMGHCTYFP |
| | | EELRSVKYAYNADLYNALNDLNNLVITRDE |
| | | NEKLEYYEKFQIIENVFKQKKKPTLKQIAK |
| | | EILVNEEDIKGYRVTSTGKPEFTNLKVYHD |
| | | IKDITARKEIIENAELLDQIAKILTIYQSS |
| | | EDIQEELTNLNSELTQEEIEQISNLKGYTG |
| | | THNLSLKAINLILDELWHTNDNQIAIFNRL |
| | | KLVPKKVDLSQQKEIPTTLVDDFILSPVVK |
| | | RSFIQSIKVINAIIKKYGLPNDIIIELARE |
| | | KNSKDAQKMINEMQKRNRQTNERIEEIIRT |
| | | TGKENAKYLIEKIKLHDMQEGKCLYSLEAI |
| | | PLEDLLNNPFNYEVDHIIPRSVSFDNSFNN |
| | | KVLVKQEENSKKGNRTPFQYLSSSDSKISY |
| | | ETFKKHILNLAKGKGRISKTKKEYLLEERD |
| | | INRFSVQKDFINRNLVDTRYATRGLMNLLR |
| | | SYFRVNNLDVKVKSINGGFTSFLRRKWKFK |
| | | KERNKGYKHHAEDALIIANADFIFKEWKKL |
| | | DKAKKVMENQMFEEKQAESMPEIETEQEYK |
| | | EIFITPHQIKHIKDFKDYKYSHRVDKKPNR |
| | | ELINDTLYSTRKDDKGNTLIVNNLNGLYDK |
| | | DNDKLKKLINKSPEKLLMYHHDPQTYQKLK |
| | | LIMEQYGDEKNPLYKYYEETGNYLTKYSKK |
| | | DNGPVIKKIKYYGNKLNAHLDITDDYPNSR |
| | | NKVVKLSLKPYRFDVYLDNGVYKFVTVKNL |
| | | DVIKKENYYEVNSKCYEEAKKLKKISNQAE |
| | | FIASFYNNDLIKINGELYRVIGVNNDLLNR |
| | | IEVNMIDITYREYLENMNDKRPPRIIKTIA |
| | | SKTQSIKKYSTDILGNLYEVKSKKHPQIIK |
| | | KG |
| SaCas9n | 255 | KRNYILGLDIGITSVGYGIIDYETRDVIDA |
| polypeptide | | GVRLFKEANVENNEGRRSKRGARRLKRRRR |
| sequence | | HRIQRVKKLLFDYNLLTDHSELSGINPYEA |
| | | RVKGLSQKLSEEEFSAALLHLAKRRGVHNV |
| | | NEVEEDTGNELSTKEQISRNSKALEEKYVA |
| | | ELQLERLKKDGEVRGSINRFKTSDYVKEAK |
| | | QLLKVQKAYHQLDQSFIDTYIDLLETRRTY |
| | | YEGPGEGSPFGWKDIKEWYEMLMGHCTYFP |
| | | EELRSVKYAYNADLYNALNDLNNLVITRDE |
| | | NEKLEYYEKFQIIENVFKQKKKPTLKQIAK |
| | | EILVNEEDIKGYRVTSTGKPEFTNLKVYHD |
| | | IKDITARKEIIENAELLDQIAKILTIYQSS |
| | | EDIQEELTNLNSELTQEEIEQISNLKGYTG |
| | | THNLSLKAINLILDELWHTNDNQIAIFNRL |
| | | KLVPKKVDLSQQKEIPTTLVDDFILSPWKR |
| | | SFIQSIKVINAIIKKYGLPNDIIIELAREK |
| | | NSKDAQKMINEMQKRNRQTNERIEEIIRTT |
| | | GKENAKYLIEKIKLHDMQEGKCLYSLEAIP |
| | | LEDLLNNPFNYEVDHIIPRSVSFDNSFNNK |
| | | VLVKQEEASKKGNRTPFQYLSSSDSKISYE |
| | | TFKKHILNLAKGKGRISKTKKEYLLEERDI |
| | | NRFSVQKDFINRNLVDTRYATRGLMNLLRS |
| | | YFRVNNLDVKVKSINGGFTSFLRRKWKFKK |
| | | ERNKGYKHHAEDALIIANADFIFKEWKKLD |
| | | KAKKVMENQMFEEKQAESMPEIETEQEYKE |
| | | IFITPHQIKHIKDFKDYKYSHRVDKKPNRE |
| | | LINDTLYSTRKDDKGNTLIVNNLNGLYDKD |
| | | NDKLKKLINKSPEKLLMYHHDPQTYQKLKL |
| | | IMEQYGDEKNPLYKYYEETGNYLTKYSKKD |
| | | NGPVIKKIKYYGNKLNAHLDITDDYPNSRN |
| | | KVVKLSLKPYRFDVYLDNGVYKFVTVKNLD |
| | | VIKKENYYEVNSKCYEEAKKLKKISNQAEF |
| | | IASFYNNDLIKINGELYRVIGVNNDLLNRI |
| | | EVNMIDITYREYLENMNDKRPPRIIKTIAS |
| | | KTQSIKKYSTDILGNLYEVKSKKHPQIIKK |
| | | G |
| SaKKH | 256 | KRNYILGLDIGITSVGYGIIDYETRDVIDA |
| Cas9 | | GVRLFKEANVENNEGRRSKRGARRLKRRRR |
| polypeptide | | HRIQRVKKLLFDYNLLTDHSELSGINPYEA |
| sequence | | RVKGLSQKLSEEEFSAALLHLAKRRGVHNV |
| | | NEVEEDTGNELSTKEQISRNSKALEEKYVA |
| | | ELQLERLKKDGEVRGSINRFKTSDYVKEAK |
| | | QLLKVQKAYHQLDQSFIDTYIDLLETRRTY |
| | | YEGPGEGSPFGWKDIKEWYEMLMGHCTYFP |
| | | EELRSVKYAYNADLYNALNDLNNLVITRDE |
| | | NEKLEYYEKFQIIENVFKQKKKPTLKQIAK |
| | | EILVNEEDIKGYRVTSTGKPEFTNLKVYHD |
| | | IKDITARKEIIENAELLDQIAKILTIYQSS |
| | | EDIQEELTNLNSELTQEEIEQISNLKGYTG |
| | | THNLSLKAINLILDELWHTNDNQIAIFNRL |
| | | KLVPKKVDLSQQKEIPTTLVDDFILSPVVK |
| | | RSFIQSIKVINAIIKKYGLPNDIIIELARE |
| | | KNSKDAQKMINEMQKRNRQTNERIEEIIRT |
| | | TGKENAKYLIEKIKLHDMQEGKCLYSLEAI |
| | | PLEDLLNNPFNYEVDHIIPRSVSFDNSFNN |
| | | KVLVKQEEASKKGNRTPFQYLSSSDSKISY |
| | | ETFKKHILNLAKGKGRISKTKKEYLLEERD |
| | | INRFSVQKDFINRNLVDTRYATRGLMNLLR |
| | | SYFRVNNLDVKVKSINGGFTSFLRRKWKFK |
| | | KERNKGYKHHAEDALIIANADFIFKEWKKL |
| | | DKAKKVMENQMFEEKQAESMPEIETEQEYK |
| | | EIFITPHQIKHIKDFKDYKYSHRVDKKPNR |
| | | KLINDTLYSTRKDDKGNTLIVNNLNGLYDK |
| | | DNDKLKKLINKSPEKLLMYHHDPQTYQKLK |
| | | LIMEQYGDEKNPLYKYYEETGNYLTKYSKK |
| | | DNGPVIKKIKYYGNKLNAHLDITDDYPNSR |
| | | NKVVKLSLKPYRFDVYLDNGVY |
| | | KFVTVKNLDVIKKENYYEVNSKCYEEAKKL |
| | | KKISNQAEFIASFYKNDLIKINGELYRVIG |
| | | VNNDLLNRIEVNMIDITYREYLENMNDKRP |
| | | PHIIKTIASKTQSIKKYSTDILGNLYEVKS |
| | | KKHPQIIKKG |
| Casphi-1 | 285 | MADTPTLFTQFLRHHLPGQRFRKDILKQAG |
| polypeptide | | RILANKGEDATIAFLRGKSEESPPDFQPPV |
| sequence | | KCPIIACSRPLTEWPIYQASVAIQGYVYGQ |
| | | SLAEFEASDPGCSKDGLLGWFDKTGVCTDY |
| | | FSVQGLNLIFQNARKRYIGVQTKVTNRNEK |
| | | RHKKLKRINAKRIAEGLPELTSDEPESALD |
| | | ETGHLIDPPGLNTNIYCYQQVSPKPLALSE |
| | | VNQLPTAYAGYSTSGDDPIQPMVTKDRLSI |
| | | SKGQPGYIPEHQRALLSQKKHRRMRGYGLK |
| | | ARALLVIVRIQDDWAVIDLRSLLRNAYWRR |
| | | IVQTKEPSTITKLLKLVTGDPVLDATRMVA |
| | | TFTYKPGIVQVRSAKCLKNKQGSKLFSERY |
| | | LNETVSVTSIDLGSNNLVAVATYRLVNGNT |
| | | PELLQRFTLPSHLVKDFERYKQAHDTLEDS |
| | | IQKTAVASLPQGQQTEIRMWSMYGFREAQE |
| | | RVCQELGLADGSIPWNVMTATSTILTDLFL |
| | | ARGGDPKKCMFTSEPKKKKNSKQVLYKIRD |
| | | RAWAKMYRTLLSKETREAWNKALWGLKR |
| | | GSPDYARLSKRKEELARRCVNYTISTAEKR |
| | | AQCGRTIVALEDLNIGFFHGRGKQEPGWVG |
| | | LFTRKKENRWLMQALHKAFLELAHHRGYHV |
| | | IEVNPAYTSQTCPVCRHCDPDNRDQHNREA |
| | | FHCIGCGFRGNADLDVATHNIAMVAITGES |
| | | LKRARGSVASKTPQPLAAE |
| Casphi-2 | 286 | MPKPAVESEFSKVLKKHFPGERFRSSYMKR |
| polypeptide | | GGKILAAQGEEAWAYLQGKSEEEPPNFQPP |
| sequence | | AKCHWTKSRDFAEWPIMKASEAIQRYIYAL |
| | | STTERAACKPGKSSESHAAWFAATGVSNHG |
| | | YSHVQGLNLIFDHTLGRYDGVLKKVQLRNE |
| | | KARARLESINASRADEGLPEIKAEEEEVAT |
| | | NETGHLLQPPGINPSFYVYQTISPQAYRPR |
| | | DEIVLPPEYAGYVRDPNAPIPLGWRNRCDI |
| | | QKGCPGYIPEWQREAGTAISPKTGKAVTVP |
| | | GLSPKKNKRMRRYWRSEKEKAQDALLVTVR |
| | | IGTDWWIDVRGLLRNARWRTIAPKDISLNA |
| | | LLDLFTGDPVIDVRRNIVTFTYTLDACGTY |
| | | ARKWTLKGKQTKATLDKLTATQTVALVAID |
| | | LGQTNPISAGISRVTQENGALQCEPLDRFT |
| | | LPDDLLKDISAYRIAWDRNEEELRARSVEA |
| | | LPEAQQAEVRALDGVSKETARTQLCADFGL |
| | | DPKRLPWDKMSSNTTFISEALLSNSVSRDQ |
| | | VFFTPAPKKGAKKKAPVEVMRKDRTWARAY |
| | | KPRLSVEAQKLKNEALWALKRTSPEYLKLS |
| | | RRKEELCRRSINYVIEKTRRRTQCQIVIPV |
| | | IEDLNVRFFHGSGKRLPGWDNFFTAKKENR |
| | | WFIQGLHKAFSDLRTHRSFYVFEVRPERTS |
| | | ITCPKCGHCEVGNRDGEAFQCLSCGKTCNA |
| | | DLDVATHNLTQVALTGKTMPKREEPRDAQG |
| | | TAPARKTKKASKSKAPPAEREDQTPAQEPS |
| | | QTS |
| Casphi-3 | 287 | MEKEITELTKIRREFPNKKFSSTDMKKAGK |
| polypeptide | | LLKAEGPDAVRDFLNSCQEIIGDFKPPVKT |
| sequence | | NIVSISRPFEEWPVSMVGRAIQEYYFSLTK |
| | | EELESVHPGTSSEDHKSFFNITGLSNYNYT |
| | | SVQGLNLIFKNAKAIYDGTLVKANNKNKKL |
| | | EKKFNEINHKRSLEGLPIITPDFEEPFDEN |
| | | GHLNNPPGINRNIYGYQGCAAKVFVPSKHK |
| | | MVSLPKEYEGYNRDPNLSLAGFRNRLEIPE |
| | | GEPGHVPWFQRMDIPEGQIGHVNKIQRFNF |
| | | VHGKNSGKVKFSDKTGRVKRYHHSKYKDAT |
| | | KPYKFLEESKKVSALDSILAIITIGDDWVV |
| | | FDIRGLYRNVFYRELAQKGLTAVQLLDLFT |
| | | GDPVIDPKKGVVTFSYKEGVVPVFSQKIVP |
| | | RFKSRDTLEKLTSQGPVALLSVDLGQNEPV |
| | | AARVCSLKNINDKITLDNSCRISFLDDYKK |
| | | QIKDYRDSLDELEIKIRLEAINSLETNQQV |
| | | EIRDLDVFSADRAKANTVDMFDIDPNLISW |
| | | DSMSDARVSTQISDLYLKNGGDESRVYFEI |
| | | NNKRIKRSDYNISQLVRPKLSDSTRKNLND |
| | | SIWKLKRTSEEYLKLSKRKLELSRAVVNYT |
| | | IRQSKLLSGINDIVIILEDLDVKKKFNGRG |
| | | IRDIGWDNFFSSRKENRWFIPAFHKAFSEL |
| | | SSNRGLCVIEVNPAWTSATCPDCGFCSKEN |
| | | RDGINFTCRKCGVSYHADIDVATLNIARVA |
| | | VLGKPMSGPADRERLGDTKKPRVARSRKTM |
| | | KRKDISNSTVEAMVTA |
| Casphi-4 | 288 | MYSLEMADLKSEPSLLAKLLRDRFPGKYWL |
| polypeptide | | PKYWKLAEKKRLTGGEEAACEYMADKQLDS |
| sequence | | PPPNFRPPARCVILAKSRPFEDWPVHRVAS |
| | | KAQSFVIGLSEQGFAALRAAPPSTADARRD |
| | | WLRSHGASEDDLMALEAQLLETIMGNAISL |
| | | HGGVLKKIDNANVKAAKRLSGRNEARLNKG |
| | | LQELPPEQEGSAYGADGLLVNPPGLNLNIY |
| | | CRKSCCPKPVKNTARFVGHYPGYLRDSDSI |
| | | LISGTMDRLTIIEGMPGHIPAWQREQGLVK |
| | | PGGRRRRLSGSESNMRQKVDPSTGPRRSTR |
| | | SGTVNRSNQRTGRNGDPLLVEIRMKEDWVL |
| | | LDARGLLRNLRWRESKRGLSCDHEDLSLSG |
| | | LLALFSGDPVIDPVRNEVVFLYGEGIIPVR |
| | | STKPVGTRQSKKLLERQASMGPLTLISCDL |
| | | GQTNLIAGRASAISLTHGSLGVRSSVRIEL |
| | | DPEIIKSFERLRKDADRLETEILTAAKETL |
| | | SDEQRGEVNSHEKDSPQTAKASLCRELGLH |
| | | PPSLPWGQMGPSTTFIADMLISHGRDDDAF |
| | | LSHGEFPTLEKRKKFDKRFCLESRPLLSSE |
| | | TRKALNESLWEVKRTSSEYARLSQRKKEMA |
| | | RRAVNFVVEISRRKTGLSNVIVNIEDLNVR |
| | | IFHGGGKQAPGWDGFFRPKSENRWFIQAIH |
| | | KAFSDLAAHHGIPVIESDPQRTSMTCPECG |
| | | HCDSKNRNGVRFLCKGCGASMDADFDAACR |
| | | NLERVALTGKPMPKPSTSCERLLSATTGKV |
| | | CSDHSLSHDAIEKAS |
| Casphi-5 | 289 | MSSLPTPLELLKQKHADLFKGLQFSSKDNK |
| polypeptide | | MAGKVLKKDGEEAALAFLSERGVSRGELPN |
| sequence | | FRPPAKTLVVAQSRPFEEFPIYRVSEAIQL |
| | | YVYSLSVKELETVPSGSSTKKEHQRFFQDS |
| | | SVPDFGYTSVQGLNKIFGLARGIYLGVITR |
| | | GENQLQKAKSKHEALNKKRRASGEAETEFD |
| | | PTPYEYMTPERKLAKPPGVNHSIMCYVDIS |
| | | VDEFDFRNPDGIVLPSEYAGYCREINTAIE |
| | | KGTVDRLGHLKGGPGYIPGHQRKESTTEGP |
| | | KINFRKGRIRRSYTALYAKRDSRRVRQGKL |
| | | ALPSYRHHMMRLNSNAESAILAVIFFGKDW |
| | | WFDLRGLLRNVRWRNLFVDGSTPSTLLGMF |
| | | GDPVIDPKRGVVAFCYKEQIVPVVSKSITK |
| | | MVKAPELLNKLYLKSEDPLVLVAIDLGQTN |
| | | PVGVGVYRVMNASLDYEWTRFALESELLRE |
| | | IESYRQRTNAFEAQIRAETFDAMTSEEQEE |
| | | ITRVRAFSASKAKENVCHRFGMPVDAVDWA |
| | | TMGSNTIHIAKWVMRHGDPSLVEVLEYRKD |
| | | NEIKLDKNGVPKKVKLTDKRIANLTSIRLR |
| | | FSQETSKHYNDTMWELRRKHPVYQKLSKSK |
| | | ADFSRRVVNSIIRRVNHLVPRARIVFIIED |
| | | LKNLGKVFHGSGKRELGWDSYFEPKSENRW |
| | | FIQVLHKAFSETGKHKGYYIIECWPNWTSC |
| | | TCPKCSCCDSENRHGEVFRCLACGYTCNTD |
| | | FGTAPDNLVKIATTGKGLPGPKKRCKGSSK |
| | | GKNPKIARSSETGVSVTESGAPKVKKSSPT |
| | | QTSQSSSQSAP |
| Casphi-6 | 290 | MNKIEKEKTPLAKLMNENFAGLRFPFAIIK |
| polypeptide | | QAGKKLLKEGELKTIEYMTGKGSIEPLPNF |
| sequence | | KPPVKCLIVAKRRDLKYFPICKASCEIQSY |
| | | VYSLNYKDFMDYFSTPMTSQKQHEEFFKKS |
| | | GLNIEYQNVAGLNLIFNNVKNTYNGVILKV |
| | | KNRNEKLKKKAIKNNYEFEEIKTFNDDGCL |
| | | INKPGINNVIYCFQSISPKILKNITHLPKE |
| | | YNDYDCSVDRNIIQKYVSRLDIPESQPGHV |
| | | PEWQRKLPEFNNTNNPRRRRKWYSNGRNIS |
| | | KGYSVDQVNQAKIEDSLLAQIKIGEDWIIL |
| | | DIRGLLRDLNRRELISYKNKLTIKDVLGFF |
| | | SDYPIIDIKKNLVTFCYKEGVIQVVSQKSI |
| | | GNKKSKQLLEKLIENKPIALVSIDLGQTNP |
| | | VSVKISKLNKINNKISIESFTYRFLNEEIL |
| | | KEIEKYRKDYDKLELKLINEA |
| Casphi-7 | 291 | MSNTAVSTREHMSNKTTPPSPLSLLLRAHF |
| polypeptide | | PGLKFESQDYKIAGKKLRDGGPEAVISYLT |
| sequence | | GKGQAKLKDVKPPAKAFVIAQSRPFIEWDL |
| | | VRVSRQIQEKIFGIPATKGRPKQDGLSETA |
| | | FNEAVASLEVDGKSKLNEETRAAFYEVLGL |
| | | DAPSLHAQAQNALIKSAISIREGVLKKVEN |
| | | RNEKNLSKTKRRKEAGEEATFVEEKAHDER |
| | | GYLIHPPGVNQTIPGYQAWIKSCPSDFIGL |
| | | PSGCLAKESAEALTDYLPHDRMTIPKGQPG |
| | | YVPEWQHP |
| | | LLNRRKNRRRRDWYSASLNKPKATCSKRSG |
| | | TPNRKNSRTDQIQSGRFKGAIPVLMRFQDE |
| | | WVIIDIRGLLRNARYRKLLKEKSTIPDLLS |
| | | LFTGDPSIDMRQGVCTFIYKAGQACSAKMV |
| | | KTKNAPEILSELTKSGPWLVSIDLGQTNPI |
| | | AAKVSRVTQLSDGQLSHETLLRELLSNDSS |
| | | DGKEIARYRVASDRLRDKLANLAVERLSPE |
| | | HKSEILRAKNDTPALCKARVCAALGLNPEM |
| | | IAWDKMTPYTEFLATAYLEKGGDRKVATLK |
| | | PKNRPEMLRRDIKFKGTEGVRIEVSPEAAE |
| | | AYREAQWDLQRTSPEYLRLSTWKQELTKRI |
| | | LNQLRHKAAKSSQCEVVVMAFEDLNIKMMH |
| | | GNGKWADGGWDAFFIKKRENRWFMQAFHKS |
| | | LTELGAHKGVPTIEVTPHRTSITCTKCGHC |
| | | DKANRDGERFACQKCGFVAHADLEIATDNI |
| | | ERVALTGKPMPKPESERSGDAKKSVGARKA |
| | | AFKPEEDAEAAE |
| Casphi-8 | 292 | MIKPTVSQFLTPGFKLIRNHSRTAGLKLKN |
| polypeptide | | EGEEACKKFVRENEIPKDECPNFQGGPAIA |
| sequence | | NIIAKSREFTEWEIYQSSLAIQEVIFTLPK |
| | | DKLPEPILKEEWRAQWLSEHGLDTVPYKEA |
| | | AGLNLIIKNAVNTYKGVQVKVDNKNKNNLA |
| | | KINRKNEIAKLNGEQEISFEEIKAFDDKGY |
| | | LLQKPSPNKSIYCYQSVSPKPFITSKYHNV |
| | | NLPEEYIGYYRKSNEPIVSPYQFDRLRIPI |
| | | GEPGYVPKWQYTFLSKKENKRRKLSKRIKN |
| | | VSPILGIICIKKDWCVFDMRGLLRTNHWKK |
| | | YHKPTDSINDLFDYFTGDPVIDTKANWRFR |
| | | YKMENGIVNYKPVREKKGKELLENICDQNG |
| | | SCKLATVDVGQNNPVAIGLFELKKVNGELT |
| | | KTLISRHPTPIDFCNKITAYRERYDKLESS |
| | | IKLDAIKQLTSEQKIEVDNYNNNFTPQNTK |
| | | QIVCSKLNINPNDLPWDKMISGTHFISEKA |
| | | QVSNKSEIYFTSTDKGKTKDVMKSDYKWFQ |
| | | DYKPKLSKEVRDALSDIEWRLRRESLEFNK |
| | | LSKSREQDARQLANWISSMCDVIGIENLVK |
| | | KNNFFGGSGKREPGWDNFYKPKKENRWWIN |
| | | AIHKALTELSQNKGKRVILLPAMRTSITCP |
| | | KCKYCDSKNRNGEKFNCLKCGIELNADIDV |
| | | ATENLATVAITAQSMPKPTCERSGDAKKPV |
| | | RARKAKAPEFHDKLAPSYTVVLREAV |
| Casphi-9 | 293 | MRSSREIGDKILMRQPAEKTAFQVFRQEVI |
| polypeptide | | GTQKLSGGDAKTAGRLYKQGKMEAAREWLL |
| sequence | | KGARDDVPPNFQPPAKCLWAVSHPFEEWDI |
| | | SKTNHDVQAYIYAQPLQAEGHLNGLSEKWE |
| | | DTSADQHKLWFEKTGVPDRGLPVQAINKIA |
| | | KAAVNRAFGVVRKVENRNEKRRSRDNRIAE |
| | | HNRENGLTEVVREAPEVATNADGFLLHPPG |
| | | IDPSILSYASVSPVPYNSSKHSFVRLPEEY |
| | | QAYNVEPDAPIPQFWEDRFAIPPGQPGYVP |
| | | EWQRLKCSTNKHRRMRQWSNQDYKPKAGRR |
| | | AKPLEFQAHLTRERAKGALLVVMRIKEDWV |
| | | VFDVRGLLRNVEWRKVLSEEAREKLTLKGL |
| | | LDLFTGDPVIDTKRGIVTFLYKAEITKILS |
| | | KRTVKTKNARDLLLRLTEPGEDGLRREVGL |
| | | VAVDLGQTHPIAAAIYRIGRTSAGALESTV |
| | | LHRQGLREDQKEKLKEYRKRHTALDSRLRK |
| | | EAFETLSVEQQKEIVTVSGSGAQITKDKVC |
| | | NYLGVDPSTLPWEKMGSYTHFISDDFLRRG |
| | | GDPNIVHFDRQPKKGKVSKKSQRIKRSDSQ |
| | | WVGRMRPRLSQETAKARMEADWAAQNENEE |
| | | YKRLARSKQELARWCVNTLLQNTRCITQCD |
| | | EIVVVIEDLNVKSLHGKGAREPGWDNFFTP |
| | | KTENRWFIQILHKTFSELPKHRGEHVIEGC |
| | | PLRTSITCPACSYCDKNSRNGEKFVCVACG |
| | | ATFHADFEVATYNLVRLATTGMPMPKSLER |
| | | QGGGEKAGGARKARKKAKQVEKIVVQANAN |
| | | VTMNGASLHSP |
| Casphi-10 | 294 | MDMLDTETNYATETPAQQQDYSPKPPKKAQ |
| polypeptide | | RAPKGFSKKARPEKKPPKPITLFTQKHFSG |
| sequence | | VRFLKRVIRDASKILKLSESRTITFLEQAI |
| | | ERDGSAPPDVTPPVHNTIMAVTRPFEEWPE |
| | | VILSKALQKHCYALTKKIKIKTWPKKGPGK |
| | | KCLAAWSARTKIPLIPGQVQATNGLFDRIG |
| | | SIYDGVEKKVTNRNANKKLEYDEAIKEGRN |
| | | PAVPEYETAYNIDGTLINKPGYNPNLYITQ |
| | | SRTPRLITEADRPLVEKILWQMVEKKTQSR |
| | | NQARRARLEKAAHLQGLPVPKFVPEKVDRS |
| | | QKIEIRIIDPLDKIEPYMPQDRMAIKASQD |
| | | GHVPYWQRPFLSKRRNRRVRAGWGKQVSSI |
| | | QAWLTGALLVIVRLGNEAFLADIRGALRNA |
| | | QWRKLLKPDATYQSLFNLFTGDPVVNTRTN |
| | | HLTMAYREGVVNIVKSRSFKGRQTREHLLT |
| | | LLGQGKTVAGVSFDLGQKHAAGLLAAHFGL |
| | | GEDGNPVFTPIQACFLPQRYLDSLTNYRNR |
| | | YDALTLDMRRQSLLALTPAQQQEFADAQRD |
| | | PGGQAKRACCLKLNLNPDEIRWDLVSGIST |
| | | MISDLYIERGGDPRDVHQQVETKPKGKRKS |
| | | EIRILKIRDGKWAYDFRPKIADETRKAQRE |
| | | QLWKLQKASSEFERLSRYKINI |
| | | ARAIANWALQWGRELSGCDIVIPVLEDLNV |
| | | GSKFFDGKGKWLLGWDNRFTPKKENRWFIK |
| | | VLHKAVAELAPHRGVPVYEVMPHRTSMTCP |
| | | ACHYCHPTNREGDRFECQSCHVVKNTDRDV |
| | | APYNILRVAVEGKTLDRWQAEKKPQAEPDR |
| | | PMILIDNQES |
| >sp\|P14739\| | 106 | MTNLSDIIEKETGKQLVIQESILMLPEEVE |
| UNGI_ | | EVIGNKPESDILVHTAYDESTDENVMLLTS |
| BPPB2 | | D APEYKPWALVIQDSNGENKIKML |
| Uracil- | | |
| DNA | | |
| glycosylase | | |
| inhibitor | | |
| Cas12b/ | 258 | MAVKSIKVKLRLDDMPEIRAGLWKLHKEVN |
| C2c1 | | AGVRYYTEWLSLLRQENLYRRSPNGDGEQE |
| | | CDKTAEECKAELLERLRARQVENGHRGPAG |
| | | SDDELLQLARQLYELLVPQAIGAKGDAQQI |
| | | ARKFLSPLADKDAVGGLGIAKAGNKPRWVR |
| | | MREAGEPGWEEEKEKAETRKSADRTADVLR |
| | | ALADFGLKPLMRVYTDSEMSSVEWKPLRKG |
| | | QAVRTWDRDMFQQAIERMMSWESWNQRVGQ |
| | | EYAKLVEQKNRFEQKNFVGQEHLVHLVNQL |
| | | QQDMKEASPGLESKEQTAHYVTGRALRGSD |
| | | KVFEKWGKLAPDAPFDLYDAEIKNVQRRNT |
| | | RRFGSHDLFAKLAEPEYQALWREDASFLTR |
| | | YAVYNSILRKLNHAKMFATFTLPDATAHPI |
| | | WTRFDKLGGNLHQYTFLFNEFGERRHAIRF |
| | | HKLLKVENGVAREVDDVTVPISMSEQLDNL |
| | | LPRDPNEPIALYFRDYGAEQHFTGEFGGAK |
| | | IQCRRDQLAHMHRRRGARDVYLNVSVRVQS |
| | | QSEARGERRPPYAAVFRLVGDNHRAFVHFD |
| | | KLSDYLAEHPDDGKLGSEGLLSGLRVMSVD |
| | | LGLRTSASISVFRVARKDELKPNSKGRVPF |
| | | FFPIKGNDNLVAVHERSQLLKLPGETESKD |
| | | LRAIREERQRTLRQLRTQLAYLRLLVRCGS |
| | | EDVGRRERSWAKLIEQPVDAANHMTPDWRE |
| | | AFENELQKLKSLHGICSDKEWMDAVYESVR |
| | | RVWRHMGKQVRDWRKDVRSGERPKIRGYAK |
| | | DVVGGNSIEQIEYLERQYKFLKSWSFFGKV |
| | | SGQVIRAEKGSRFAITLREHIDHAKEDRLK |
| | | KLADRIIMEALGYVYALDERGKGKWVAKYP |
| | | PCQLILLEELSEYQFNNDRPPSENNQLMQW |
| | | SHRGVFQELINQAQVHDLLVGTMYAAFSSR |
| | | FDARTGAPGIRCRRVPARCTQEHNPEPFPV |
| | | WVLNKFWEHTLDACPLRADDLIPTGEGEIF |
| | | VSPFSAEEGDFHQIHADLNAAQNLQQRLWS |
| | | DFDISQIRLRCDWGEVDGELVLIPRLTGKR |
| | | TADSYSNKVFYTNTGVTYYERERGKKRRKV |
| | | FAQEKLSEEEAELLVEADEAREKSWLMRDP |
| | | SGIINRGNWTRQKEFWSMVNQRIEGYLVKQ |
| | | IRSRVPLQDSACENTGDI |
| high | 1423 | MDKKYSIGLAIGTNSVGWAVITDEYKVPSK |
| fidelity | | KFKVLGNTDRHSIKKNLIGALLFDSGETAE |
| Cas9 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTAFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | ALSRKLINGIRDKQSGKTILDFLKSDGFAN |
| | | RNFMALIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRAITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFDSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| Wt Cas9 | 233 | ATGGATAAAAAGTATTCTATTGGTTTAGAC |
| domain | | ATCGGCACTAATTCCGTTGGATGGGCTGTC |
| | | ATAACCGATGAATACAAAGTACCTTCAAAG |
| | | AAATTTAAGGTGTTGGGGAACACAGACCGT |
| | | CATTCGATTAAAAAGAATCTTATCGGTGCC |
| | | CTCCTATTCGATAGTGGCGAAACGGCAGAG |
| | | GCGACTCGCCTGAAACGAACCGCTCGGAGA |
| | | AGGTATACACGTCGCAAGAACCGAATATGT |
| | | TACTTACAAGAAATTTTTAGCAATGAGATG |
| | | GCCAAAGTTGACGATTCTTTCTTTCACCGT |
| | | TTGGAAGAGTCCTTCCTTGTCGAAGAGGAC |
| | | AAGAAACATGAACGGCACCCCATCTTTGGA |
| | | AACATAGTAGATGAGGTGGCATATCATGAA |
| | | AAGTACCCAACGATTTATCACCTCAGAAAA |
| | | AAGCTAGTTGACTCAACTGATAAAGCGGAC |
| | | CTGAGGTTAATCTACTTGGCTCTTGCCCAT |
| | | ATGATAAAGTTCCGTGGGCACTTTCTCATT |
| | | GAGGGTGATCTAAATCCGGACAACTCGGAT |
| | | GTCGACAAACTGTTCATCCAGTTAGTACAA |
| | | ACCTATAATCAGTTGTTTGAAGAGAACCCT |
| | | ATAAATGCAAGTGGCGTGGATGCGAAGGCT |
| | | ATTCTTAGCGCCCGCCTCTCTAAATCCCGA |
| | | CGGCTAGAAAACCTGATCGCACAATTACCC |
| | | GGAGAGAAGAAAAATGGGTTGTTCGGTAAC |
| | | CTTATAGCGCTCTCACTAGGCCTGACACCA |
| | | AATTTTAAGTCGAACTTCGACTTAGCTGAA |
| | | GATGCCAAATTGCAGCTTAGTAAGGACACG |
| | | TACGATGACGATCTCGACAATCTACTGGCA |
| | | CAAATTGGAGATCAGTATGCGGACTTATTT |
| | | TTGGCTGCCAAAAACCTTAGCGATGCAATC |
| | | CTCCTATCTGACATACTGAGAGTTAATACT |
| | | GAGATTACCAAGGCGCCGTTATCCGCTTCA |
| | | ATGATCAAAAGGTACGATGAACATCACCAA |
| | | GACTTGACACTTCTCAAGGCCCTAGTCCGT |
| | | CAGCAACTGCCTGAGAAATATAAGGAAATA |
| | | TTCTTTGATCAGTCGAAAAACGGGTACGCA |
| | | GGTTATATTGACGGCGGAGCGAGTCAAGAG |
| | | GAATTCTACAAGTTTATCAAACCCATATTA |
| | | GAGAAGATGGATGGGACGGAAGAGTTGCTT |
| | | GTAAAACTCAATCGCGAAGATCTACTGCGA |
| | | AAGCAGCGGACTTTCGACAACGGTAGCATT |
| | | CCACATCAAATCCACTTAGGCGAATTGCAT |
| | | GCTATACTTAGAAGGCAGGAGGATTTTTAT |
| | | CCGTTCCTCAAAGACAATCGTGAAAAGATT |
| | | GAGAAAATCCTAACCTTTCGCATACCTTAC |
| | | TATGTGGGACCCCTGGCCCGAGGGAACTCT |
| | | CGGTTCGCATGGATGACAAGAAAGTCCGAA |
| | | GAAACGATTACTCCATGGAATTTTGAGGAA |
| | | GTTGTCGATAAAGGTGCGTCAGCTCAATCG |
| | | TTCATCGAGAGGATGACCAACTTTGACAAG |
| | | AATTTACCGAACGAAAAAGTATTGCCTAAG |
| | | CACAGTTTACTTTACGAGTATTTCACAGTG |
| | | TACAATGAACTCACGAAAGTTAAGTATGTC |
| | | ACTGAGGGCATGCGTAAACCCGCCTTTCTA |
| | | AGCGGAGAACAGAAGAAAGCAATAGTAGAT |
| | | CTGTTATTCAAGACCAACCGCAAAGTGACA |
| | | GTTAAGCAATTGAAAGAGGACTACTTTAAG |
| | | AAAATTGAATGCTTCGATTCTGTCGAGATC |
| | | TCCGGGGTAGAAGATCGATTTAATGCGTCA |
| | | CTTGGTACGTATCATGACCTCCTAAAGATA |
| | | ATTAAAGATAAGGACTTCCTGGATAACGAA |
| | | GAGAATGAAGATATCTTAGAAGATATAGTG |
| | | TTGACTCTTACCCTCTTTGAAGATCGGGAA |
| | | ATGATTGAGGAAAGACTAAAAACATACGCT |
| | | CACCTGTTCGACGATAAGGTTATGAAACAG |
| | | TTAAAGAGGCGTCGCTATACGGGCTGGGGA |
| | | CGATTGTCGCGGAAACTTATCAACGGGATA |
| | | AGAGACAAGCAAAGTGGTAAAACTATTCTC |
| | | GATTTTCTAAAGAGCGACGGCTTCGCCAAT |
| | | AGGAACTTTATGCAGCTGATCCATGATGAC |
| | | TCTTTAACCTTCAAAGAGGATATACAAAAG |
| | | GCACAGGTTTCCGGACAAGGGGACTCATTG |
| | | CACGAACATATTGCGAATCTTGCTGGTTCG |
| | | CCAGCCATCAAAAAGGGCATACTCCAGACA |
| | | GTCAAAGTAGTGGATGAGCTAGTTAAGGTC |
| | | ATGGGACGTCACAAACCGGAAAACATTGTA |
| | | ATCGAGATGGCACGCGAAAATCAAACGACT |
| | | CAGAAGGGGCAAAAAAACAGTCGAGAGCGG |
| | | ATGAAGAGAATAGAAGAGGGTATTAAAGAA |
| | | CTGGGCAGCCAGATCTTAAAGGAGCATCCT |
| | | GTGGAAAATACCCAATTGCAGAACGAGAAA |
| | | CTTTACCTCTATTACCTACAAAATGGAAGG |
| | | GACATGTATGTTGATCAGGAACTGGACATA |
| | | AACCGTTTATCTGATTACGACGTCGATCAC |
| | | ATTGTACCCCAATCCTTTTTGAAGGACGAT |
| | | TCAATCGACAATAAAGTGCTTACACGCTCG |
| | | GATAAGAACCGAGGGAAAAGTGACAATGTT |
| | | CCAAGCGAGGAAGTCGTAAAGAAAATGAAG |
| | | AACTATTGGCGGCAGCTCCTAAATGCGAAA |
| | | CTGATAACGCAAAGAAAGTTCGATAACTTA |
| | | ACTAAAGCTGAGAGGGGTGGCTTGTCTGAA |
| | | CTTGACAAGGCCGGATTTATTAAACGTCAG |
| | | CTCGTGGAAACCCGCCAAATCACAAAGCAT |
| | | GTTGCACAGATACTAGATTCCCGAATGAAT |
| | | ACGAAATACGACGAGAACGATAAGCTGATT |
| | | CGGGAAGTCAAAGTAATCACTTTAAAGTCA |
| | | AAATTGGTGTCGGACTTCAGAAAGGATTTT |
| | | CAATTCTATAAAGTTAGGGAGATAAATAAC |
| | | TACCACCATGCGCACGACGCTTATCTTAAT |
| | | GCCGTCGTAGGGACCGCACTCATTAAGAAA |
| | | TACCCGAAGCTAGAAAGTGAGTTTGTGTAT |
| | | GGTGATTACAAAGTTTATGACGTCCGTAAG |
| | | ATGATCGCGAAAAGCGAACAGGAGATAGGC |
| | | AAGGCTACAGCCAAATACTTCTTTTATTCT |
| | | AACATTATGAATTTCTTTAAGACGGAAATC |
| | | ACTCTGGCAAACGGAGAGATACGCAAACGA |
| | | CCTTTAATTGAAACCAATGGGGAGACAGGT |
| | | GAAATCGTATGGGATAAGGGCCGGGACTTC |
| | | GCGACGGTGAGAAAAGTTTTGTCCATGCCC |
| | | CAAGTCAACATAGTAAAGAAAACTGAGGTG |
| | | CAGACCGGAGGGTTTTCAAAGGAATCGATT |
| | | CTTCCAAAAAGGAATAGTGATAAGCTCATC |
| | | GCTCGTAAAAAGGACTGGGACCCGAAAAAG |
| | | TACGGTGGCTTCGATAGCCCTACAGTTGCC |
| | | TATTCTGTCCTAGTAGTGGCAAAAGTTGAG |
| | | AAGGGAAAATCCAAGAAACTGAAGTCAGTC |
| | | AAAGAATTATTGGGGATAACGATTATGGAG |
| | | CGCTCGTCTTTTGAAAAGAACCCCATCGAC |
| | | TTCCTTGAGGCGAAAGGTTACAAGGAAGTA |
| | | AAAAAGGATCTCATAATTAAACTACCAAAG |
| | | TATAGTCTGTTTGAGTTAGAAAATGGCCGA |
| | | AAACGGATGTTGGCTAGCGCCGGAGAGCTT |
| | | CAAAAGGGGAACGAACTCGCACTACCGTCT |
| | | AAATACGTGAATTTCCTGTATTTAGCGTCC |
| | | CATTACGAGAAGTTGAAAGGTTCACCTGAA |
| | | GATAACGAACAGAAGCAACTTTTTGTTGAG |
| | | CAGCACAAACATTATCTCGACGAAATCATA |
| | | GAGCAAATTTCGGAATTCAGTAAGAGAGTC |
| | | ATCCTAGCTGATGCCAATCTGGACAAAGTA |
| | | TTAAGCGCATACAACAAGCACAGGGATAAA |
| | | CCCATACGTGAGCAGGCGGAAAATATTATC |
| | | CATTTGTTTACTCTTACCAACCTCGGCGCT |
| | | CCAGCCGCATTCAAGTATTTTGACACAACG |
| | | ATAGATCGCAAACGATACACTTCTACCAAG |
| | | GAGGTGCTAGACGCGACACTGATTCACCAA |
| | | TCCATCACGGGATTATATGAAACTCGGATA |
| | | GATTTGTCACAGCTTGGGGGTGACGGATCC |
| | | CCCAAGAAGAAGAGGAAAGTCTCGAGCGAC |
| | | TACAAAGACCATGACGGTGATTATAAAGAT |
| | | CATGACATCGATTACAAGGATGACGATGAC |
| | | AAGGCTGCAGGA |
| wild-type | 234 | MDKKYSIGLAIGTNSVGWAVITDEYKVPSK |
| Cas9 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEWKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFDSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| PAM- | 1304 | MDKKYSIGLAIGTNSVGWAVITDEYKVPSK |
| binding | | KFKVLGNTDRHSIKKNLIGALLFDSGETAE |
| SpEQR Cas9 | | ATRLKRTARRRYTRRKNRICYLQEIFSNEM |
| polypeptide | | AKVDDSFFHRLEESVLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFESPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKQYRSTKEVLDATLIHQSI |
| | | TGLYETRIDLSQLGGD |
| PAM- | 1305 | MDKKYSIGLAIGTNSVGWAVITDEYKVPSK |
| binding | | KFKVLGNTDRHSIKKNLIGALLFDSGETAE |
| SpVQR Cas9 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFVSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSPEDNEQKQLFVEQ |
| | | HKHYLDEIIEQISEFSKRVILADANLDKVL |
| | | SAYNKHRDKPIREQAENIIHLFTLTNLGAP |
| | | AAFKYFDTTIDRKQYRSTKEVLDATLIHQS |
| | | ITGLYETRIDLSQLGGD |
| SpVQR Cas9 | 1306 | MDKKYSIGLAIGTNSVGWAVITDEYKVPSK |
| polypeptide | | KFKVLGNTDRHSIKKNLIGALLFDSGETAE |
| sequence | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEWKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFVSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASARELQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKEYRSTKEVLDATLIHQSI |
| | | TGLYETRIDLSQLGGD |
| SpyMacCas9 | 1307 | MDKKYSIGLDIGTNSVGWAVITDDYKVPSK |
| polypeptide | | KFKVLGNTDRHSIKKNLIGALLFGSGETAE |
| sequence | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLADSTDKAD |
| | | LRLIYLALAHMIKFRGHFLIEGDLNPDNSD |
| | | VDKLFIQLVQIYNQLFEENPINASRVDAKA |
| | | ILSARLSKSRRLENLIAQLPGEKRNGLFGN |
| | | LIALSLGLTPNFKSNFDLAEDAKLQLSKDT |
| | | YDDDLDNLLAQIGDQYADLFLAAKNLSDAI |
| | | LLSDILRVNSEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | WDKGASAQSFIERMTNFDKNLPNEKVLPKH |
| | | SLLYEYFTVYNELTKVKYVTEGMRKPAFLS |
| | | GEQKKAIVDLLFKTNRKVTVKQLKEDYFKK |
| | | IECFDSVEISGVEDRFNASLGAYHDLLKII |
| | | KDKDFLDNEENEDILEDIVLTLTLFEDRGM |
| | | IEERLKTYAHLFDDKVMKQLKRRRYTGWGR |
| | | LSRKLINGIRDKQSGKTILDFLKSDGFANR |
| | | NFMQLIHDDSLTFKEDIQKAQVSGQGHSLH |
| | | EQIANLAGSPAIKKGILQTVKIVDELVKVM |
| | | GHKPENIVIEMARENQTTQKGQKNSRERMK |
| | | RIEEGIKELGSQILKEHPVENTQLQNEKLY |
| | | LYYLQNGRDMYVDQELDINRLSDYDVDHIV |
| | | PQSFIKDDSIDNKVLTRSDKNRGKSDNVPS |
| | | EEWKKMKNYWRQLLNAKLITQRKFDNLTKA |
| | | ERGGLSELDKAGFIKRQLVETRQITKHVAQ |
| | | ILDSRMNTKYDENDKLIREVKVITLKSKLV |
| | | SDFRKDFQFYKVREINNYHHAHDAYLNAVV |
| | | GTALIKKYPKLESEFVYGDYKVYDVRKMIA |
| | | KSEQEIGKATAKYFFYSNIMNFFKTEITLA |
| | | NGEIRKRPLIETNGETGEIVWDKGRDFATV |
| | | RKVLSMPQVNIVKKTEIQTVGQNGGLFDDN |
| | | PKSPLEVTPSKLVPLKKELNPKKYGGYQKP |
| | | TTAYPVLLITDTKQLIPISVMNKKQFEQNP |
| | | VKFLRDRGYQQVGKNDFIKLPKYTLVDIGD |
| | | GIKRLWASSKEIHKGNQLVVSKKSQILLYH |
| | | AHHLDSDLSNDYLQNHNQQFDVLFNEIISF |
| | | SKKCKLGKEHIQKIENVYSNKKNSASIEEL |
| | | AESFIKLLGFTQLGATSPFNFLGVKLNQKQ |
| | | YKGKKDYILPCTEGTLIRQSITGLYETRVD |
| | | LSKIGED |
| CP5 | 257 | EIGKATAKYFFYSNIMNFFKTEITLANGEI |
| polypeptide | | RKRPLIETNGETGEIVWDKGRDFATVRKVL |
| sequence | | SMPQVNIVKKTEVQTGGFSKESILPKRNSD |
| | | KLIARKKDWDPKKYGGFMQPTVAYSVLVVA |
| | | KVEKGKSKKLKSVKELLGITIMERSSFEKN |
| | | PIDFLEAKGYKEVKKDLIIKLPKYSLFELE |
| | | NGRKRMLASAKFLQKGNELALPSKYVNFLY |
| | | LASHYEKLKGSPEDNEQKQLFVEQHKHYLD |
| | | EIIEQISEFSKRVILADANLDKVLSAYNKH |
| | | RDKPIREQAENIIHLFTLTNLGAPRAFKYF |
| | | DTTIARKEYRSTKEVLDATLIHQSITGLYE |
| | | TRIDLSQLGGDGGSGGSGGSGGSGGSGGSG |
| | | GMDKKYSIGLAIGTNSVGWAVITDEYKVPS |
| | | KKFKVLGNTDRHSIKKNLIGALLFDSGETA |
| | | EATRLKRTARRRYTRRKNRICYLQEIFSNE |
| | | MAKVDDSFFHRLEESFLVEEDKKHERHPIF |
| | | GNIVDEVAYHEKYPTIYHLRKKLVDSTDKA |
| | | DLRLIYLALAHMIKFRGHFLIEGDLNPDNS |
| | | DVDKLFIQLVQTYNQLFEENPINASGVDAK |
| | | AILSARLSKSRRLENLIAQLPGEKKNGLFG |
| | | NLIALSLGLTPNFKSNFDLAEDAKLQLSKD |
| | | TYDDDLDNLLAQIGDQYADLFLAAKNLSDA |
| | | ILLSDILRVNTEITKAPLSASMIKRYDEHH |
| | | QDLTLLKALVRQQLPEKYKEIFFDQSKNGY |
| | | AGYIDGGASQEEFYKFIKPILEKMDGTEEL |
| | | LVKLNREDLLRKQRTFDNGSIPHQIHLGEL |
| | | HAILRRQEDFYPFLKDNREKIEKILTFRIP |
| | | YYVGPLARGNSRFAWMTRKSEETITPWNFE |
| | | EVVDKGASAQSFIERMTNFDKNLPNEKVLP |
| | | KHSLLYEYFTVYNELTKVKYVTEGMRKPAF |
| | | LSGEQKKAIVDLLFKTNRKVTVKQLKEDYF |
| | | KKIECFDSVEISGVEDRFNASLGTYHDLLK |
| | | IIKDKDFLDNEENEDILEDIVLTLTLFEDR |
| | | EMIEERLKTYAHLFDDKVMKQLKRRRYTGW |
| | | GRLSRKLINGIRDKQSGKTILDFLKSDGFA |
| | | NRNFMQLIHDDSLTFKEDIQKAQVSGQGDS |
| | | LHEHIANLAGSPAIKKGILQTVKWDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEWKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLESEFVYGDYKVYDVRKM |
| | | IAKSEQEGADKRTADGSEFESPKKKRKV |
| Cas12c1 | 266 | MQTKKTHLHLISAKASRKYRRTIACLSDTA |
| polypeptide | | KKDLERRKQSGAADPAQELSCLKTIKFKLE |
| sequence | | VPEGSKLPSFDRISQIYNALETIEKGSLSY |
| | | LLFALILSGFRIFPNSSAAKTFASSSCYKN |
| | | DQFASQIKEIFGEMVKNFIPSELESILKKG |
| | | RRKNNKDWTEENIKRVLNSEFGRKNSEGSS |
| | | ALFDSFLSKFSQELFRKFDSWNEVNKKYLE |
| | | AAELLDSMLASYGPFDSVCKMIGDSDSRNS |
| | | LPDKSTIAFTNNAEITVDIESSVMPYMAIA |
| | | ALLREYRQSKSKAAPVAYVQSHLTTTNGNG |
| | | LSWFFKFGLDLIRKAPVSSKQSTSDGSKSL |
| | | QELFSVPDDKLDGLKFIKEACEALPEASLL |
| | | CGEKGELLGYQDFRTSFAGHIDSWVANYVN |
| | | RLFELIELVNQLPESIKLPSILTQKNHNLV |
| | | ASLGLQEAEVSHSLELFEGLVKNVRQTLKK |
| | | LAGIDISSSPNEQDIKEFYAFSDVLNRLGS |
| | | IRNQIENAVQTAKKDKIDLESAIEWKEWKK |
| | | LKKLPKLNGLGGGVPKQQELLDKALESVKQ |
| | | IRHYQRIDFERVIQWAVNEHCLETVPKFLV |
| | | DAEKKKINKESSTDFAAKENAVRFLLEGIG |
| | | AAARGKTDSVSKAAYNWFVVNNFLAKKDLN |
| | | RYFINCQGCIYKPPYSKRRSLAFALRSDNK |
| | | DTIEVVWEKFETFYKEISKEIEKFNIFSQE |
| | | FQTFLHLENLRMKLLL |
| | | RRIQKPIPAEIAFFSLPQEYYDSLPPNVAF |
| | | LALNQEITPSEYITQFNLYSSFLNGNLILL |
| | | RRSRSYLRAKFSWVGNSKLIYAAKEARLWK |
| | | IPNAYWKSDEWKMILDSNVLVFDKAGNVLP |
| | | APTLKKVCEREGDLRLFYPLLRQLPHDWCY |
| | | RNPFVKSVGREKNVIEVNKEGEPKVASALP |
| | | GSLFRLIGPAPFKSLLDDCFFNPLDKDLRE |
| | | CMLIVDQEISQKVEAQKVEASLESCTYSIA |
| | | VPIRYHLEEPKVSNQFENVLAIDQGEAGLA |
| | | YAVFSLKSIGEAETKPIAVGTIRIPSIRRL |
| | | IHSVSTYRKKKQRLQNFKQNYDSTAFIMRE |
| | | NVTGDVCAKIVGLMKEFNAFPVLEYDVKNL |
| | | ESGSRQLSAVYKAVNSHFLYFKEPGRDALR |
| | | KQLWYGGDSWTIDGIEIVTRERKEDGKEGV |
| | | EKIVPLKVFPGRSVSARFTSKTCSCCGRNV |
| | | FDWLFTEKKAKTNKKFNVNSKGELTTADGV |
| | | IQLFEADRSKGPKFYARRKERTPLTKPIAK |
| | | GSYSLEEIERRVRTNLRRAPKSKQSRDTSQ |
| | | SQYFCVYKDCALHFSGMQADENAAINIGRR |
| | | FLTALRKNRRSDFPSNVKISDRLLDN |
| Cas12c2 | 267 | MTKHSIPLHAFRNSGADARKWKGRIALLAK |
| polypeptide | | RGKETMRTLQFPLEMSEPEAAAINTTPFAV |
| sequence | | AYNAIEGTGKGTLFDYWAKLHLAGFRFFPS |
| | | GGAATIFRQQAVFEDASWNAAFCQQSGKDW |
| | | PWLVPSKLYERFTKAPREVAKKDGSKKSIE |
| | | FTQENVANESHVSLVGASITDKTPEDQKEF |
| | | FLKMAGALAEKFDSWKSANEDRIVAMKVID |
| | | EFLKSEGLHLPSLENIAVKCSVETKPDNAT |
| | | VAWHDAPMSGVQNLAIGVFATCASRIDNIY |
| | | DLNGGKLSKLIQESATTPNVTALSWLFGKG |
| | | LEYFRTTDIDTIMQDFNIPASAKESIKPLV |
| | | ESAQAIPTMTVLGKKNYAPFRPNFGGKIDS |
| | | WIANYASRLMLLNDILEQIEPGFELPQALL |
| | | DNETLMSGIDMTGDELKELIEAVYAWVDAA |
| | | KQGLATLLGRGGNVDDAVQTFEQFSAMMDT |
| | | LNGTLNTISARYVRAVEMAGKDEARLEKLI |
| | | ECKFDIPKWCKSVPKLVGISGGLPKVEEEI |
| | | KVMNAAFKDVRARMFVRFEEIAAYVASKGA |
| | | GMDVYDALEKRELEQIKKLKSAVPERAHIQ |
| | | AYRAVLHRIGRAVQNCSEKTKQLFSSKVIE |
| | | MGVFKNPSHLNNFIFNQKGAIYRSPFDRSR |
| | | HAPYQLHADKLLKNDWLELLAEISATLMAS |
| | | ESTEQMEDALRLERTRLQLQLSGLPDWEYP |
| | | ASLAKPDIEVEIQTALKMQLAKDTVTSDVL |
| | | QRAFNLYSSVLSGLTFKLLRRSFSLKMRFS |
| | | VADTTQLIYVPKVCDWAIPKQYLQAEGEIG |
| | | IAARWTESSPAKMVTEVEMKEPKALGHFMQ |
| | | QAPHDWYFDASLGGTQVAGRIVEKGKEVGK |
| | | ERKLVGYRMRGNSAYKTVLDKSLVGNTELS |
| | | QCSMIIEIPYTQTVDADFRAQVQAGLPKVS |
| | | INLPVKETITASNKDEQMLFDRFVAIDLGE |
| | | RGLGYAVFDAKTLELQESGHRPIKAITNLL |
| | | NRTHHYEQRPNQRQKFQAKFNVNLSELREN |
| | | TVGDVCHQINRICAYYNAFPVLEYMVPDRL |
| | | DKQLKSVYESVTNRYIWSSTDAHKSARVQF |
| | | WLGGETWEHPYLKSAKDKKPLVLSPGRGAS |
| | | GKGTSQTCSCCGRNPFDLIKDMKPRAKIAV |
| | | VDGKAKLENSELKLFERNLESKDDMLARRH |
| | | RNERAGMEQPLTPGNYTVDEIKALLRANLR |
| | | RAPKNRRTKDTTVSEYHCVFSDCGKTMHAD |
| | | ENAAVNIGGKFIADIEK |
| OspCas12c | 268 | MTKLRHRQKKLTHDWAGSKKREVLGSNGKL |
| polypeptide | | QNPLLMPVKKGQVTEFRKAFSAYARATKGE |
| sequence | | MTDGRKNMFTHSFEPFKTKPSLHQCELADK |
| | | AYQSLHSYLPGSLAHFLLSAHALGFRIFSK |
| | | SGEATAFQASSKIEAYESKLASELACVDLS |
| | | IQNLTISTLFNALTTSVRGKGEETSADPLI |
| | | ARFYTLLTGKPLSRDTQGPERDLAEVISRK |
| | | IASSFGTWKEMTANPLQSLQFFEEELHALD |
| | | ANVSLSPAFDVLIKMNDLQGDLKNRTIVFD |
| | | PDAPVFEYNAEDPADIIIKLTARYAKEAVI |
| | | KNQNVGNYVKNAITTTNANGLGWLLNKGLS |
| | | LLPVSTDDELLEFIGVERSHPSCHALIELI |
| | | AQLEAPELFEKNVFSDTRSEVQGMIDSAVS |
| | | NHIARLSSSRNSLSMDSEELERLIKSFQIH |
| | | TPHCSLFIGAQSLSQQLESLPEALQSGVNS |
| | | ADILLGSTQYMLTNSLVEESIATYQRTLNR |
| | | INYLSGVAGQINGAIKRKAIDGEKIHLPAA |
| | | WSELISLPFIGQPVIDVESDLAHLKNQYQT |
| | | LSNEFDTLISALQKNFDLNFNKALLNRTQH |
| | | FEAMCRSTKKNALSKPEIVSYRDLLARLTS |
| | | CLYRGSLVLRRAGIEVLKKHKIFESNSELR |
| | | EHVHERKHFVFVSPLDRKAKKLLRLTDSRP |
| | | DLLHVIDEILQHDNLENKDRESLWLVRSGY |
| | | LLAGLPDQLSSSFINLPIITQKGDRRLIDL |
| | | IQYDQINRDAFVMLVTSAFKSNLSGLQYRA |
| | | NKQSFVVTRTLSPYLGSKLVYVPKDKDWLV |
| | | PSQMFEGRFADILQSDYMVWKDAGRLCVID |
| | | TAKHLSNIKKSVFSSEEVLAFLRELPHRTF |
| | | IQTEVRGLGVNVDGIAFNNGDIPSLKTFSN |
| | | CVQVKVSRTNTSLVQTLNRWFEGGKVSPPS |
| | | IQFERAYYKKDDQIHEDAAKRKIRFQMPAT |
| | | ELVHASDDAGWTPSYLLGIDPGEYGMGLSL |
| | | VSINNGEVLDSGFIHINSLINFASKKSNHQ |
| | | TKVVPRQQYKSPYANYLEQSKDSAAGDIAH |
| | | ILDRLIYKLNALPVFEALSGNSQSAADQVW |
| | | TKVLSFYTWGDNDAQNSIRKQHWFGASHWD |
| | | IKGMLRQPPTEKKPKPYIAFPGSQVSSYGN |
| | | SQRCSCCGRNPIEQLREMAKDTSIKELKIR |
| | | NSEIQLFDGTIKLFNPDPSTVIERRRHNLG |
| | | PSRIPVADRTFKNISPSSLEFKELITIVSR |
| | | SIRHSPEFIAKKRGIGSEYFCAYSDCNSSL |
| | | NSEANAAANVAQKFQKQLFFEL |
| Cas12g1 | 269 | MAQASSTPAVSPRPRPRYREERTLVRKLLP |
| polypeptide | | RPGQSKQEFRENVKKLRKAFLQFNADVSGV |
| sequence | | CQWAIQFRPRYGKPAEPTETFWKFFLEPET |
| | | SLPPNDSRSPEFRRLQAFEAAAGINGAAAL |
| | | DDPAFTNELRDSILAVASRPKTKEAQRLFS |
| | | RLKDYQPAHRMILAKVAAEWIESRYRRAHQ |
| | | NWERNYEEWKKEKQEWEQNHPELTPEIREA |
| | | FNQIFQQLEVKEKRVRICPAARLLQNKDNC |
| | | QYAGKNKHSVLCNQFNEFKKNHLQGKAIKF |
| | | FYKDAEKYLRCGLQSLKPNVQGPFREDWNK |
| | | YLRYMNLKEETLRGKNGGRLPHCKNLGQEC |
| | | EFNPHTALCKQYQQQLSSRPDLVQHDELYR |
| | | KWRREYWREPRKPVFRYPSVKRHSIAKIFG |
| | | ENYFQADFKNSVVGLRLDSMPAGQYLEFAF |
| | | APWPRNYRPQPGETEISSVHLHFVGTRPRI |
| | | GFRFRVPHKRSRFDCTQEELDELRSRTFPR |
| | | KAQDQKFLEAARKRLLETFPGNAEQELRLL |
| | | AVDLGTDSARAAFFIGKTFQQAFPLKIVKI |
| | | EKLYEQWPNQKQAGDRRDASSKQPRPGLSR |
| | | DHVGRHLQKMRAQASEIAQKRQELTGTPAP |
| | | ETTTDQAAKKATLQPFDLRGLTVHTARMIR |
| | | DWARLNARQIIQLAEENQVDLIVLESLRGF |
| | | RPPGYENLDQEKKRRVAFFAHGRIRRKVTE |
| | | KAVERGMRVVTVPYLASSKVCAECRKKQKD |
| | | NKQWEKNKKRGLFKCEGCGSQAQVDENAAR |
| | | VLGRVFWGEIELPTAIP |
| Cas12h1 | 270 | MKVHEIPRSQLLKIKQYEGSFVEWYRDLQE |
| polypeptide | | DRKKFASLLFRWAAFGYAAREDDGATYISP |
| sequence | | SQALLERRLLLGDAEDVAIKFLDVLFKGGA |
| | | PSSSCYSLFYEDFALRDKAKYSGAKREFIE |
| | | GLATMPLDKIIERIRQDEQLSKIPAEEWLI |
| | | LGAEYSPEEIWEQVAPRIVNVDRSLGKQLR |
| | | ERLGIKCRRPHDAGYCKILMEVVARQLRSH |
| | | NETYHEYLNQTHEMKTKVANNLTNEFDLVC |
| | | EFAEVLEEKNYGLGWYVLWQGVKQALKEQK |
| | | KPTKIQIAVDQLRQPKFAGLLTAKWRALKG |
| | | AYDTWKLKKRLEKRKAFPYMPNWDNDYQIP |
| | | VGLTGLGVFTLEVKRTEVWDLKEHGKLFCS |
| | | HSHYFGDLTAEKHPSRYHLKFRHKLKLRKR |
| | | DSRVEPTIGPWIEAALREITIQKKPNGVFY |
| | | LGLPYALSHGIDNFQIAKRFFSAAKPDKEV |
| | | INGLPSEMWGAADLNLSNIVAPVKARIGKG |
| | | LEGPLHALDYGYGELIDGPKILTPDGPRCG |
| | | ELISLKRDIVEIKSAIKEFKACQREGLTMS |
| | | EETTTWLSEVESPSDSPRCMIQSRIADTSR |
| | | RLNSFKYQMNKEGYQDLAEALRLLDAMDSY |
| | | NSLLESYQRMHLSPGEQSPKEAKFDTKRAS |
| | | FRDLLRRRVAHTIVEYFDDCDIVFFEDLDG |
| | | PSDSDSRNNALVKLLSPRTLLLYIRQALEK |
| | | RGIGMVEVAKDGTSQNNPISGHVGWRNKQN |
| | | KSEIYFYEDKELLVMDADEVGAMNILCRGL |
| | | NHSVCPYSFVTKAPEKKNDEKKEGDYGKRV |
| | | KRFLKDRYGSSNVRFLVASMGFVTVTTKRP |
| | | KDALVGKRLYYHGGELVTHDLHNRMKDEIK |
| | | YLVEKEVLARRVSLSDSTIKSYKSFAHV |
| Cas12i1 | 271 | MSNKEKNASETRKAYTTKMIPRSHDRMKLL |
| polypeptide | | GNFMDYLMDGTPIFFELWNQFGGGIDRDII |
| sequence | | SGTANKDKISDDLLLAVNWFKVMPINSKPQ |
| | | GVSPSNLANLFQQYSGSEPDIQAQEYFASN |
| | | FDTEKHQWKDMRVEYERLLAELQLSRSDMH |
| | | HDLKLMYKEKCIGLSLSTAHYITSVMFGTG |
| | | AKNNRQTKHQFYSKVIQLLEESTQINSVEQ |
| | | LASIILKAGDCDSYRKLRIRCSRKGATPSI |
| | | LKIVQDYELGTNHDDEVNVPSLIANLKEKL |
| | | GRFEYECEWKCMEKIKAFLASKVGPYYLGS |
| | | YSAMLENALSPIKGMTTKNCKFVLKQIDAK |
| | | NDIKYENEPFGKIVEGFFDSPYFESDTNVK |
| | | WVLHPHHIGESNIKTLWEDLNAIHSKYEED |
| | | IASLSEDKKEKRIKVYQGDVCQTINTYCEE |
| | | VGKEAKTPLVQLLRYLYSRKDDIAVDKIID |
| | | GITFLSKKHKVEKQKINPVIQKYPSFNFGN |
| | | NSKLLGKIISPKDKLKHNLKCNRNQVDNYI |
| | | WIEIKVLNTKTMRWEKHHYALSSTRFLEEV |
| | | YYPATSENPPDALAARFRTKTNGYEGKPAL |
| | | SAEQIEQIRSAPVGLRKVKKRQMRLEAARQ |
| | | QNLLPRYTWGKDFNINICKRGNNFEVTLAT |
| | | KVKKKKEKNYKVVLGYDANIVRKNTYAAIE |
| | | AHANGDGVIDYNDLPVKPIESGFVTVESQV |
| | | RDKSYDQLSYNGVKLLYCKPHVESRRSFLE |
| | | KYRNGTMKDNRGNNIQIDFMKDFEAIADDE |
| | | TSLYYFNMKYCKLLQSSIRNHSSQAKEYRE |
| | | EIFELLRDGKLSVLKLSSLSNLSFVMFKVA |
| | | KSLIGTYFGHLLKKPKNSKSDVKAPPITDE |
| | | DKQKADPEMFALRLALEEKRLNKVKSKKEV |
| | | IANKIVAKALELRDKYGPVLIKGENISDTT |
| | | KKGKKSSTNSFLMDWLARGVANKVKEMVMM |
| | | HQGLEFVEVNPNFTSHQDPFVHKNPENTFR |
| | | ARYSRCTPSELTEKNRKEILSFLSDKPSKR |
| | | PTNAYYNEGAMAFLATYGLKKNDVLGVSLE |
| | | KFKQIMANILHQRSEDQLLFPSRGGMFYLA |
| | | TYKLDADATSVNWNGKQFWVCNADLVAAYN |
| | | VGLVDIQKDFKKK |
| Cas12i2 | 272 | MSSAIKSYKSVLRPNERKNQLLKSTIQCLE |
| polypeptide | | DGSAFFFKMLQGLFGGITPEIVRFSTEQEK |
| sequence | | QQQDIALWCAVNWFRPVSQDSLTHTIASDN |
| | | LVEKFEEYYGGTASDAIKQYFSASIGESYY |
| | | WNDCRQQYYDLCRELGVEVSDLTHDLEILC |
| | | REKCLAVATESNQNNSIISVLFGTGEKEDR |
| | | SVKLRITKKILEAISNLKEIPKNVAPIQEI |
| | | ILNVAKATKETFRQVYAGNLGAPSTLEKFI |
| | | AKDGQKEFDLKKLQTDLKKVIRGKSKERDW |
| | | CCQEELRSYVEQNTIQYDLWAWGEMFNKAH |
| | | TALKIKSTRNYNFAKQRLEQFKEIQSLNNL |
| | | LWKKLNDFFDSEFFSGEETYTICVHHLGGK |
| | | DLSKLYKAWEDDPADPENAIVVLCDDLKNN |
| | | FKKEPIRNILRYIFTIRQECSAQDILAAAK |
| | | YNQQLDRYKSQKANPSVLGNQGFTWTNAVI |
| | | LPEKAQRNDRPNSLDLRIWLYLKLRHPDGR |
| | | WKKHHIPFYDTRFFQEIYAAGNSPVDTCQF |
| | | RTPRFGYHLPKLTDQTAIRVNKKHVKAAKT |
| | | EARIRLAIQQGTLPVSNLKITEISATINSK |
| | | GQVRIPVKFDVGRQKGTLQIGDRFCGYDQN |
| | | QTASHAYSLWEVVKEGQYHKELGCFVRFIS |
| | | SGDIVSITENRGNQFDQLSYEGLAYPQYAD |
| | | WRKKASKFVSLWQITKKNKKKEIVTVEAKE |
| | | KFDAICKYQPRLYKFNKEYAYLLRDIVRGK |
| | | SLVELQQIRQEIFRFIEQDCGVTRLGSLSL |
| | | STLETVKAVKGIIYSYFSTALNASKNNPIS |
| | | DEQRKEFDPELFALLEKLELIRTRKKKQKV |
| | | ERIANSLIQTCLENNIKFIRGEGDLSTTNN |
| | | ATKKKANSRSMDWLARGVFNKIRQLAPMHN |
| | | ITLFGCGSLYTSHQDPLVHRNPDKAMKCRW |
| | | AAIPVKDIGDWVLRKLSQNLRAKNIGTGEY |
| | | YHQGVKEFLSHYELQDLEEELLKWRSDRKS |
| | | NIPCWVLQNRLAEKLGNKEAWYIPVRGGRI |
| | | YFATHKVATGAVSIVFDQKQVWVCNADHVA |
| | | AANIALTVKGIGEQSSDEENPDGSRIKLQL |
| | | TS |
| Linker | 1308 | (GGGS)N |
| Linker | 109 | (GGGGS)N |
| Linker | 1309 | (EAAAK)N |
| Linker | 56 | SGSETPGTSESATPES |
| | 57 | (SGGS)N |
| Linker | 273 | GGSGGS |
| Linker | 1310 | GSSGSETPGTSESATPESSG |
| Linker | 1311 | GGAGGCTCTGGAGGAAGC |
| Linker | 1312 | GGCTCTTCTGGATCTGAAACACCTGGCACA |
| | | AGCGAGAGCGCCACCCCTGAGAGCTCTGGC |
| AacCas12b | 259 | MAVKSMKVKLRLDNMPEIRAGLWKLHTEVN |
| polypeptide | | AGVRYYTEWLSLLRQENLYRRSPNGDGEQE |
| sequence | | CYKTAEECKAELLERLRARQVENGHCGPAG |
| | | SDDELLQLARQLYELLVPQAIGAKGDAQQI |
| | | ARKFLSPLADKDAVGGLGIAKAGNKPRWVR |
| | | MREAGEPGWEEEKAKAEARKSTDRTADVLR |
| | | ALADFGLKPLMRVYTDSDMSSVQWKPLRKG |
| | | QAVRTWDRDMFQQAIERMMSWESWNQRVGE |
| | | AYAKLVEQKSRFEQKNFVGQEHLVQLVNQL |
| | | QQDMKEASHGLESKEQTAHYLTGRALRGSD |
| | | KVFEKWEKLDPDAPFDLYDTEIKNVQRRNT |
| | | RRFGSHDLFAKLAEPKYQALWREDASFLTR |
| | | YAVYNSIVRKLNHAKMFATFTLPDATAHPI |
| | | WTRFDKLGGNLHQYTFLFNEFGEGRHAIRF |
| | | QKLLTVEDGVAKEVDDVTVPISMSAQLDDL |
| | | LPRDPHELVALYFQDYGAEQHLAGEFGGAK |
| | | IQYRRDQLNHLHARRGARDVYLNLSVRVQS |
| | | QSEARGERRPPYAAVFRLVGDNHRAFVHFD |
| | | KLSDYLAEHPDDGKLGSEGLLSGLRVMSVD |
| | | LGLRTSASISVFRVARKDELKPNSEGRVPF |
| | | CFPIEGNENLVAVHERSQLLKLPGETESKD |
| | | LRAIREERQRTLRQLRTQLAYLRLLVRCGS |
| | | EDVGRRERSWAKLIEQPMDANQMTPDWREA |
| | | FEDELQKLKSLYGICGDREWTEAVYESVRR |
| | | VWRHMGKQVRDWRKDVRSGERPKIRGYQKD |
| | | VVGGNSIEQIEYLERQYKFLKSWSFFGKVS |
| | | GQVIRAEKGSRFAITLREHIDHAKEDRLKK |
| | | LADRIIMEALGYVYALDDERGKGKWVAKYP |
| | | PCQLILLEELSEYQFNNDRPPSENNQLMQW |
| | | SHRGVFQELLNQAQVHDLLVGTMYAAFSSR |
| | | FDARTGAPGIRCRRVPARCAREQNPEPFPW |
| | | WLNKFVAEHKLDGCPLRADDLIPTGEGEFF |
| | | VSPFSAEEGDFHQIHADLNAAQNLQRRLWS |
| | | DFDISQIRLRCDWGEVDGEPVLIPRTTGKR |
| | | TADSYGNKVFYTKTGVTYYERERGKKRRKV |
| | | FAQEELSEEEAELLVEADEAREKSVVLMRD |
| | | PSGIINRGDWTRQKEFWSMVNQRIEGYLVK |
| | | QIRSRVRLQESACENTGDI |
| BhCas12b | 260 | MAPKKKRKVGIHGVPAAATRSFILKIEPNE |
| polypeptide | | EVKKGLWKTHEVLNHGIAYYMNILKLIRQE |
| sequence | | AIYEHHEQDPKNPKKVSKAEIQAELWDFVL |
| | | KMQKCNSFTHEVDKDEVFNILRELYEELVP |
| | | SSVEKKGEANQLSNKFLYPLVDPNSQSGKG |
| | | TASSGRKPRWYNLKIAGDPSWEEEKKKWEE |
| | | DKKKDPLAKILGKLAEYGLIPLFIPYTDSN |
| | | EPIVKEIKWMEKSRNQSVRRLDKDMFIQAL |
| | | ERFLSWESWNLKVKEEYEKVEKEYKTLEER |
| | | IKEDIQALKALEQYEKERQEQLLRDTLNTN |
| | | EYRLSKRGLRGWREIIQKWLKMDENEPSEK |
| | | YLEVFKDYQRKHPREAGDYSVYEFLSKKEN |
| | | HFIWRNHPEYPYLYATFCEIDKKKKDAKQQ |
| | | ATFTLADPINHPLWVRFEERSGSNLNKYRI |
| | | LTEQLHTEKLKKKLTVQLDRLIYPTESGGW |
| | | EEKGKVDIVLLPSRQFYNQIFLDIEEKGKH |
| | | AFTYKDESIKFPLKGTLGGARVQFDRDHLR |
| | | RYPHKVESGNVGRIYFNMTVNIEPTESPVS |
| | | KSLKIHRDDFPKVVNFKPKELTEWIKDSKG |
| | | KKLKSGIESLEIGLRVMSIDLGQRQAAAAS |
| | | IFEVVDQKPDIEGKLFFPIKGTELYAVHRA |
| | | SFNIKLPGETLVKSREVLRKAREDNLKLMN |
| | | QKLNFLRNVLHFQQFEDITEREKRVTKWIS |
| | | RQENSDVPLVYQDELIQIRELMYKPYKDWV |
| | | AFLKQLHKRLEVEIGKEVKHWRKSLSDGRK |
| | | GLYGISLKNIDEIDRTRKFLLRWSLRPTEP |
| | | GEVRRLEPGQRFAIDQLNHLNALKEDRLKK |
| | | MANTIIMHALGYCYDVRKKKWQAKNPACQI |
| | | ILFEDLSNYNPYEERSRFENSKLMKWSRRE |
| | | IPRQVALQGEIYGLQVGEVGAQFSSRFHAK |
| | | TGSPGIRCSWTKEKLQDNRFFKNLQREGRL |
| | | TLDKIAVLKEGDLYPDKGGEKFISLSKDRK |
| | | CVTTHADINAAQNLQKRFWTRTHGFYKVYC |
| | | KAYQVDGQTVYIPESKDQKQKIIEEFGEGY |
| | | FILKDGVYEWVNAGKLKIKKGSSKQSSSEL |
| | | VDSDILKDSFDLASELKGEKLMLYRDPSGN |
| | | VFPSDKWMAAGVFFGKLERILISKLTNQYS |
| | | ISTIEDDSSKQSMKRPAATKKAGQAKKKK |
| BvCas12b | 264 | MAIRSIKLKMKTNSGTDSIYLRKALWRTHQ |
| (Bacillus | | LINEGIAYYMNLLTLYRQEAIGDKTKEAYQ |
| sp. V3-13) | | AELINIIRNQQRNNGSSEEHGSDQEILALL |
| poly- | | RQLYELIIPSSIGESGDANQLGNKFLYPLV |
| nucleotide | | DPNSQSGKGTSNAGRKPRWKRLKEEGNPDW |
| sequence | | ELEKKKDEERKAKDPTVKIFDNLNKYGLLP |
| | | LFPLFTNIQKDIEWLPLGKRQSVRKWDKDM |
| | | FIQAIERLLSWESWNRRVADEYKQLKEKTE |
| | | SYYKEHLTGGEEWIEKIRKFEKERNMELEK |
| | | NAFAPNDGYFITSRQIRGWDRVYEKWSKLP |
| | | ESASPEELWKWAEQQNKMSEGFGDPKVFSF |
| | | LANRENRDIWRGHSERIYHIAAYNGLQKKL |
| | | SRTKEQATFTLPDAIEHPLWIRYESPGGTN |
| | | LNLFKLEEKQKKNYYVTLSKIIWPSEEKWI |
| | | EKENIEIPLAPSIQFNRQIKLKQHVKGKQE |
| | | ISFSDYSSRISLDGVLGGSRIQFNRKYIKN |
| | | HKELLGEGDIGPVFFNLWDVAPLQETRNGR |
| | | LQSPIGKALKVISSDFSKVIDYKPKELMDW |
| | | MNTGSASNSFGVASLLEGMRVMSIDMGQRT |
| | | SASVSIFEVVKELPKDQEQKLFYSINDTEL |
| | | FAIHKRSFLLNLPGEVVTKNNKQQRQERRK |
| | | KRQFVRSQIRMLANVLRLETKKTPDERKKA |
| | | IHKLMEIVQSYDSWTASQKEVWEKELNLLT |
| | | NMAAFNDEIWKESLVELHHRIEPYVGQIVS |
| | | KWRKGLSEGRKNLAGISMWNIDELEDTRRL |
| | | LISWSKRSRTPGEANRIETDEPFGSSLLQH |
| | | IQNVKDDRLKQMANLIIMTALGFKYDKEEK |
| | | DRYKRWKETYPACQIILFENLNRYLFNLDR |
| | | SRRENSRLMKWAHRSIPRTVSMQGEMFGLQ |
| | | VGDVRSEYSSRFHAKTGAPGIRCHALTEED |
| | | LKAGSNTLKRLIEDGFINESELAYLKKGDI |
| | | IPSQGGELFVTLSKRYKKDSDNNELTVIHA |
| | | DINAAQNLQKRFWQQNSEVYRVPCQLARMG |
| | | EDKLYIPKSQTETIKKYFGKGSFVKNNTEQ |
| | | EVYKWEKSEKMKIKTDTTFDLQDLDGFEDI |
| | | SKTIELAQEQQKKYLTMFRDPSGYFFNNET |
| | | WRPQKEYWSIVNNIIKSCLKKKILSNKVEL |
| BTCas12b. | 265 | MATRSFILKIEPNEEVKKGLWKTHEVLNHG |
| BTCas1 | | IAYYMNILKLIRQEAIYEHHEQDPKNPKKV |
| 2b | | SKAEIQAELWDFVLKMQKCNSFTHEVDKDV |
| polypeptide | | VFNILRELYEELVPSSVEKKGEANQLSNKF |
| sequence | | LYPLVDPNSQSGKGTASSGRKPRWYNLKIA |
| | | GDPSWEEEKKKWEEDKKKDPLAKILGKLAE |
| | | YGLIPLFIPFTDSNEPIVKEIKWMEKSRNQ |
| | | SVRRLDKDMFIQALERFLSWESWNLKVKEE |
| | | YEKVEKEHKTLEERIKEDIQAFKSLEQYEK |
| | | ERQEQLLRDTLNTNEYRLSKRGLRGWREII |
| | | QKWLKMDENEPSEKYLEVFKDYQRKHPREA |
| | | GDYSVYEFLSKKENHFIWRNHPEYPYLYAT |
| | | FCEIDKKKKDAKQQATFTLADPINHPLWVR |
| | | FEERSGSNLNKYRILTEQLHTEKLKKKLTV |
| | | QLDRLIYPTESGGWEEKGKVDIVLLPSRQF |
| | | YNQIFLDIEEKGKHAFTYKDESIKFPLKGT |
| | | LGGARVQFDRDHLRRYPHKVESGNVGRIYF |
| | | NMTVNIEPTESPVSKSLKIHRDDFPKFVNF |
| | | KPKELTEWIKDSKGKKLKSGIESLEIGLRV |
| | | MSIDLGQRQAAAASIFEWDQKPDIEGKLFF |
| | | PIKGTELYAVHRASFNIKLPGETLVKSREV |
| | | LRKAREDNLKLMNQKLNFLRNVLHFQQFED |
| | | ITEREKRVTKWISRQENSDVPLVYQDELIQ |
| | | IRELMYKPYKDWVAFLKQLHKRLEVEIGKE |
| | | VKHWRKSLSDGRKGLYGISLKNIDEIDRTR |
| | | KFLLRWSLRPTEPGEVRRLEPGQRFAIDQL |
| | | NHLNALKEDRLKKMANTIIMHALGYCYDVR |
| | | KKKWQAKNPACQIILFEDLSNYNPYEERSR |
| | | FENSKLMKWSRREIPRQVALQGEIYGLQVG |
| | | EVGAQFSSRFHAKTGSPGIRCSVVTKEKLQ |
| | | DNRFFKNLQREGRLTLDKIAVLKEGDLYPD |
| | | KGGEKFISLSKDRKLVTTHADINAAQNLQK |
| | | RFWTRTHGFYKVYCKAYQVDGQTVYIPESK |
| | | DQKQKIIEEFGEGYFILKDGVYEWGNAGKL |
| | | KIKKGSSKQSSSELVDSDILKDSFDLASEL |
| | | KGEKLMLYRDPSGNVFPSDKWMAAGVFFGK |
| | | LERILISKLTNQYSISTIEDDSSKQSM |
| 5′UTR | 261 | GGGAAATAAGAGAGAAAAGAAGAGTAAGAA |
| | | GAAATATAAGAGCCACC |
| 3′UTR | 262 | GCTGGAGCCTCGGTGGCCATGCTTCTTGCC |
| (TriLink | | CCTTGGGCCTCCCCCCAGCCCCTCCTCCCC |
| standard | | TTCCTGCACCCGTACCCCCGTGGTCTTTGA |
| UTR) | | ATAAAGTCTGA |
| bhCas12b | 263 | ATGGCCCCAAAGAAGAAGCGGAAGGTCGGT |
| (V4) | | ATCCACGGAGTCCCAGCAGCCGCCACCAGA |
| poly- | | TCCTTCATCCTGAAGATCGAGCCCAACGAG |
| nucleotide | | GAAGTGAAGAAAGGCCTCTGGAAAACCCAC |
| sequence | | GAGGTGCTGAACCACGGAATCGCCTACTAC |
| | | ATGAATATCCTGAAGCTGATCCGGCAAGAG |
| | | GCCATCTACGAGCACCACGAGCAGGACCCC |
| | | AAGAATCCCAAGAAGGTGTCCAAGGCCGAG |
| | | ATCCAGGCCGAGCTGTGGGATTTCGTGCTG |
| | | AAGATGCAGAAGTGCAACAGCTTCACACAC |
| | | GAGGTGGACAAGGACGAGGTGTTCAACATC |
| | | CTGAGAGAGCTGTACGAGGAACTGGTGCCC |
| | | AGCAGCGTGGAAAAGAAGGGCGAAGCCAAC |
| | | CAGCTGAGCAACAAGTTTCTGTACCCTCTG |
| | | GTGGACCCCAACAGCCAGTCTGGAAAGGGA |
| | | ACAGCCAGCAGCGGCAGAAAGCCCAGATGG |
| | | TACAACCTGAAGATTGCCGGCGATCCCTCC |
| | | TGGGAAGAAGAGAAGAAGAAGTGGGAAGAA |
| | | GATAAGAAAAAGGACCCGCTGGCCAAGATC |
| | | CTGGGCAAGCTGGCTGAGTACGGACTGATC |
| | | CCTCTGTTCATCCCCTACACCGACAGCAAC |
| | | GAGCCCATCGTGAAAGAAATCAAGTGGATG |
| | | GAAAAGTCCCGGAACCAGAGCGTGCGGCGG |
| | | CTGGATAAGGACATGTTCATTCAGGCCCTG |
| | | GAACGGTTCCTGAGCTGGGAGAGCTGGAAC |
| | | CTGAAAGTGAAAGAGGAATACGAGAAGGTC |
| | | GAGAAAGAGTACAAGACCCTGGAAGAGAGG |
| | | ATCAAAGAGGACATCCAGGCTCTGAAGGCT |
| | | CTGGAACAGTATGAGAAAGAGCGGCAAGAA |
| | | CAGCTGCTGCGGGACACCCTGAACACCAAC |
| | | GAGTACCGGCTGAGCAAGAGAGGCCTTAGA |
| | | GGCTGGCGGGAAATCATCCAGAAATGGCTG |
| | | AAAATGGACGAGAACGAGCCCTCCGAGAAG |
| | | TACCTGGAAGTGTTCAAGGACTACCAGCGG |
| | | AAGCACCCTAGAGAGGCCGGCGATTACAGC |
| | | GTGTACGAGTTCCTGTCCAAGAAAGAGAAC |
| | | CACTTCATCTGGCGGAATCACCCTGAGTAC |
| | | CCCTACCTGTACGCCACCTTCTGCGAGATC |
| | | GACAAGAAAAAGAAGGACGCCAAGCAGCAG |
| | | GCCACCTTCACACTGGCCGATCCTATCAAT |
| | | CACCCTCTGTGGGTCCGATTCGAGGAAAGA |
| | | AGCGGCAGCAACCTGAACAAGTACAGAATC |
| | | CTGACCGAGCAGCTGCACACCGAGAAGCTG |
| | | AAGAAAAAGCTGACAGTGCAGCTGGACCGG |
| | | CTGATCTACCCTACAGAATCTGGCGGCTGG |
| | | GAAGAGAAGGGCAAAGTGGACATTGTGCTG |
| | | CTGCCCAGCCGGCAGTTCTACAACCAGATC |
| | | TTCCTGGACATCGAGGAAAAGGGCAAGCAC |
| | | GCCTTCACCTACAAGGATGAGAGCATCAAG |
| | | TTCCCTCTGAAGGGCACACTCGGCGGAGCC |
| | | AGAGTGCAGTTCGACAGAGATCACCTGAGA |
| | | AGATACCCTCACAAGGTGGAAAGCGGCAAC |
| | | GTGGGCAGAATCTACTTCAACATGACCGTG |
| | | AACATCGAGCCTACAGAGTCCCCAGTGTCC |
| | | AAGTCTCTGAAGATCCACCGGGACGACTTC |
| | | CCCAAGGTGGTCAACTTCAAGCCCAAAGAA |
| | | CTGACCGAGTGGATCAAGGACAGCAAGGGC |
| | | AAGAAACTGAAGTCCGGCATCGAGTCCCTG |
| | | GAAATCGGCCTGAGAGTGATGAGCATCGAC |
| | | CTGGGACAGAGACAGGCCGCTGCCGCCTCT |
| | | ATTTTCGAGGTGGTGGATCAGAAGCCCGAC |
| | | ATCGAAGGCAAGCTGTTTTTCCCAATCAAG |
| | | GGCACCGAGCTGTATGCCGTGCACAGAGCC |
| | | AGCTTCAACATCAAGCTGCCCGGCGAGACA |
| | | CTGGTCAAGAGCAGAGAAGTGCTGCGGAAG |
| | | GCCAGAGAGGACAATCTGAAACTGATGAAC |
| | | CAGAAGCTCAACTTCCTGCGGAACGTGCTG |
| | | CACTTCCAGCAGTTCGAGGACATCACCGAG |
| | | AGAGAGAAGCGGGTCACCAAGTGGATCAGC |
| | | AGACAAGAGAACAGCGACGTGCCCCTGGTG |
| | | TACCAGGATGAGCTGATCCAGATCCGCGAG |
| | | CTGATGTACAAGCCTTACAAGGACTGGGTC |
| | | GCCTTCCTGAAGCAGCTCCACAAGAGACTG |
| | | GAAGTCGAGATCGGCAAAGAAGTGAAGCAC |
| | | TGGCGGAAGTCCCTGAGCGACGGAAGAAAG |
| | | GGCCTGTACGGCATCTCCCTGAAGAACATC |
| | | GACGAGATCGATCGGACCCGGAAGTTCCTG |
| | | CTGAGATGGTCCCTGAGGCCTACCGAACCT |
| | | GGCGAAGTGCGTAGACTGGAACCCGGCCAG |
| | | AGATTCGCCATCGACCAGCTGAATCACCTG |
| | | AACGCCCTGAAAGAAGATCGGCTGAAGAAG |
| | | ATGGCCAACACCATCATCATGCACGCCCTG |
| | | GGCTACTGCTACGACGTGCGGAAGAAGAAA |
| | | TGGCAGGCTAAGAACCCCGCCTGCCAGATC |
| | | ATCCTGTTCGAGGATCTGAGCAACTACAAC |
| | | CCCTACGAGGAAAGGTCCCGCTTCGAGAAC |
| | | AGCAAGCTCATGAAGTGGTCCAGACGCGAG |
| | | ATCCCCAGACAGGTTGCACTGCAGGGCGAG |
| | | ATCTATGGCCTGCAAGTGGGAGAAGTGGGC |
| | | GCTCAGTTCAGCAGCAGATTCCACGCCAAG |
| | | ACAGGCAGCCCTGGCATCAGATGTAGCGTC |
| | | GTGACCAAAGAGAAGCTGCAGGACAATCGG |
| | | TTCTTCAAGAATCTGCAGAGAGAGGGCAGA |
| | | CTGACCCTGGACAAAATCGCCGTGCTGAAA |
| | | GAGGGCGATCTGTACCCAGACAAAGGCGGC |
| | | GAGAAGTTCATCAGCCTGAGCAAGGATCGG |
| | | AAGTGCGTGACCACACACGCCGACATCAAC |
| | | GCCGCTCAGAACCTGCAGAAGCGGTTCTGG |
| | | ACAAGAACCCACGGCTTCTACAAGGTGTAC |
| | | TGCAAGGCCTACCAGGTGGACGGCCAGACC |
| | | GTGTACATCCCTGAGAGCAAGGACCAGAAG |
| | | CAGAAGATCATCGAAGAGTTCGGCGAGGGC |
| | | TACTTCATTCTGAAGGACGGGGTGTACGAA |
| | | TGGGTCAACGCCGGCAAGCTGAAAATCAAG |
| | | AAGGGCAGCTCCAAGCAGAGCAGCAGCGAG |
| | | CTGGTGGATAGCGACATCCTGAAAGACAGC |
| | | TTCGACCTGGCCTCCGAGCTGAAAGGCGAA |
| | | AAGCTGATGCTGTACAGGGACCCCAGCGGC |
| | | AATGTGTTCCCCAGCGACAAATGGATGGCC |
| | | GCTGGCGTGTTCTTCGGAAAGCTGGAACGC |
| | | ATCCTGATCAGCAAGCTGACCAACCAGTAC |
| | | TCCATCAGCACCATCGAGGACGACAGCAGC |
| | | AAGCAGTCTATGAAAAGGCCGGCGGCCACG |
| | | AAAAAGGCCGGCCAGGCAAAAAAGAAAAAG |
| NLS | 1313 | MAPKKKRKVGTHGVPAA |
| NLS | 1314 | ATGGCCCCAAAGAAGAAGCGGAAGGTCGGT |
| | | ATCCACGGAGTCCCAGCAGCC |
| 101 Cas9 | 1315 | MDKKYSTGLATGTNSVGWAVTTDEYKVPSK |
| TadAins | | KFKVLGNTDRHSTKKNLTGALLFDSGETAE |
| 1015 | | ATRLKRTARRRYTRRKNRTCYLQETFSNEM |
| polypeptide | | AKVDDSFFHRLEESFLVEEDKKHERHPTFG |
| sequence | | NTVDEVAYHEKYPTTYHLRKKLVDSTDKAD |
| | | LRLTYLALAHMTKFRGHFLTEGDLNPDNSD |
| | | VDKLFTQLVQTYNQLFEENPTNASGVDAKA |
| | | TLSARLSKSRRLENLTAQLPGEKKNGLFGN |
| | | LTALSLGLTPNFKSNFDLAEDAKLQLSKDT |
| | | YDDDLDNLLAQTG |
| | | DQYADLFLAAKNLSDAILLSDILRVNTEIT |
| | | KAPLSASMIKRYDEHHQDLTLLKALVRQQL |
| | | PEKYKEIFFDQSKNGYAGYIDGGASQEEFY |
| | | KFIKPILEKMDGTEELLVKLNREDLLRKQR |
| | | TFDNGSIPHQIHLGELHAILRRQEDFYPFL |
| | | KDNREKIEKILTFRIPYYVGPLARGNSRFA |
| | | WMTRKSEETITPWNFEEVVDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKWDELVKVMGRHKPENIVIEMA |
| | | RENQTTQKGQKNSRERMKRIEEGIKELGSQ |
| | | ILKEHPVENTQLQNEKLYLYYLQNGRDMYV |
| | | DQELDINRLSDYDVDHIVPQSFLKDDSIDN |
| | | KVLTRSDKNRGKSDNVPSEEVVKKMKNYWR |
| | | QLLNAKLITQRKFDNLTKAERGGLSELDKA |
| | | GFIKRQLVETRQITKHVAQILDSRMNTKYD |
| | | ENDKLIREVKVITLKSKLVSDFRKDFQFYK |
| | | VREINNYHHAHDAYLNAVVGTALIKKYPKLE |
| | | SEFVYGDYKVGSSGSETPGTSESATPESSG |
| | | SEVEFSHEYWMRHALTLAKRARDEREVPVG |
| | | AVLVLNNRVIGEGWNRAIGLHDPTAHAEIM |
| | | ALRQGGLVMQNYRLIDATLYVTFEPCVMCA |
| | | GAMIHSRIGRWFGVRNAKTGAAGSLMDVLH |
| | | YPGMNHRVEITEGILADECAALLCYFFRMP |
| | | RQVFNAQKKAQSSTDYDVRKMIAKSEQEIG |
| | | KATAKYFFYSNIMNFFKTEITLANGEIRKR |
| | | PLIETNGETGEIWVDKGRDFATVRKVLSMP |
| | | QVNIVKKTEVQTGGFSKESILPKRNSDKLI |
| | | ARKKDWDPKKYGGFDSPTVAYSVLVVAKVE |
| | | KGKSKKLKSVKELLGITIMERSSFEKNPID |
| | | FLEAKGYKEVKKDLIIKLPKYSLFELENGR |
| | | KRMLASAGELQKGNELALPSKYVNFLYLAS |
| | | HYEKLKGSPEDNEQKQLFVEQHKHYLDEII |
| | | EQISEFSKRVILADANLDKVLSAYNKHRDK |
| | | PIREQAENIIHLFTLTNLGAPAAFKYFDTT |
| | | IDRKRYTSTKEVLDATLIHQSITGLYETRI |
| | | DLSQLGGD |
| 102 Cas9 | 1316 | MDKKYSIGLAIGTNSVGWAVITDEYKVPSK |
| TadAins | | KFKVLGNTDRHSIKKNLIGALLFDSGETAE |
| 1022 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | GSSGSETPGTSESATPESSGSEVEFSHEYW |
| | | MRHALTLAKRARDEREVPVGAVLVLNNRVI |
| | | GEGWNRAIGLHDPTAHAEIMALRQGGLVMQ |
| | | NYRLIDATLYVTFEPCVMCAGAMIHSRIGR |
| | | VVFGVRNAKTGAAGSLMDVLHYPGMNHRVE |
| | | ITEGILADECAALLCYFFRMPRQVFNAQKK |
| | | AQSSTDAKSEQEIGKATAKYFFYSNIMNFF |
| | | KTEITLANGEIRKRPLIETNGETGEIVWDK |
| | | GRDFATVRKVLSMPQVNIVKKTEVQTGGFS |
| | | KESILPKRNSDKLIARKKDWDPKKYGGFDS |
| | | PTVAYSVLWAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGD |
| 103 Cas9 | 1317 | MDKKYSIGLAIGTNSVGWAVITDEYKVPSK |
| TadAins | | KFKVLGNTDRHSIKKNLIGALLFDSGETAE |
| 1029 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGSSGSETPGTSESATPESSGSE |
| | | VEFSHEYWMRHALTLAKRARDEREVPVGAV |
| | | LVLNNRVIGEGWNRAIGLHDPTAHAEIMAL |
| | | RQGGLVMQNYRLIDATLYVTFEPCVMCAGA |
| | | MIHSRIGRVVFGVRNAKTGAAGSLMDVLHY |
| | | PGMNHRVEITEGILADECAALLCYFFRMPR |
| | | QVFNAQKKAQSSTDGKATAKYFFYSNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLVVAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 103 Cas9 | 1318 | MDKKYSIGLAIGTNSVGWAVITDEYKVPSK |
| TadAins | | KFKVLGNTDRHSIKKNLIGALLFDSGETAE |
| 1040 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGKATAKLDEIIEQISEFSKRVI |
| | | LADANLDKVLSAYNKHRDKPIREQAENIIH |
| | | LFTLTNLGAPAAFKYFDTTIDRKRYTSTKE |
| | | VLDATLIHQSITGLYETRIDLSQLGGD |
| 103 Cas9 | 1317 | MDKKYSIGLAIGTNSVGWAVITDEYKVPSK |
| TadAins | | KFKVLGNTDRHSIKKNLIGALLFDSGETAE |
| 1029 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGSSGSETPGTSESATPESSGSE |
| | | VEFSHEYWMRHALTLAKRARDEREVPVGAV |
| | | LVLNNRVIGEGWNRAIGLHDPTAHAEIMAL |
| | | RQGGLVMQNYRLIDATLYVTFEPCVMCAGA |
| | | MIHSRIGRVVFGVRNAKTGAAGSLMDVLHY |
| | | PGMNHRVEITEGILADECAALLCYFFRMPR |
| | | QVFNAQKKAQSSTDGKATAKYFFYSNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLVVAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 103 Cas9 | 1318 | MDKKYSIGLAIGTNSVGWAVITDEYKVPSK |
| TadAins | | KFKVLGNTDRHSIKKNLIGALLFDSGETAE |
| 1040 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | IAKSEQEIGKATAKYFFYSGSSGSETPGTS |
| | | ESATPESSGSEVEFSHEYWMRHALTLAKRA |
| | | RDEREVPVGAVLVLNNRVIGEGWNRAIGLH |
| | | DPTAHAEIMALRQGGLVMQNYRLIDATLYV |
| | | TFEPCVMCAGAMIHSRIGRVVFGVRNAKTG |
| | | AAGSLMDVLHYPGMNHRVEITEGILADECA |
| | | ALLCYFFRMPRQVFNAQKKAQSSTDNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLWAKVEKGKSKKLKSVKELLGI |
| | | TIMERSSFEKNPIDFLEAKGYKEVKKDLII |
| | | KLPKYSLFELENGRKRMLASAGELQKGNEL |
| | | ALPSKYVNFLYLASHYEKLKGSPEDNEQKQ |
| | | LFVEQHKHYLDEIIEQISEFSKRVILADAN |
| | | LDKVLSAYNKHRDKPIREQAENIIHLFTLT |
| | | NLGAPAAFKYFDTTIDRKRYTSTKEVLDAT |
| | | LIHQSITGLYETRIDLSQLGGD |
| 105 Cas9 | 1319 | MDKKYSIGLAIGTNSVGWAVITDEYKVPSK |
| TadAins | | KFKVLGNTDRHSIKKNLIGALLFDSGETAE |
| 1068 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGEIRKRPLIETNGEGSSGSETPGTSESA |
| | | TPESSGSEVEFSHEYWMRHALTLAKRARDE |
| | | REVPVGAVLVLNNRVIGEGWNRAIGLHDPT |
| | | AHAEIMALRQGGLVMQNYRLIDATLYVTFE |
| | | PCVMCAGAMIHSRIGRVVFGVRNAKTGAAG |
| | | SLMDVLHYPGMNHRVEITEGILADECAALL |
| | | CYFFRMPRQVFNAQKKAQSSTDTGEIVWDK |
| | | GRDFATVRKVLSMPQVNIVKKTEVQTGGFS |
| | | KESILPKRNSDKLIARKKDWDPKKYGGFDS |
| | | PTVAYSVLWAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGD |
| 106 Cas9 | 1320 | MDKKYSIGLAIGTNSVGWAVITDEYKVPSK |
| TadAins | | KFKVLGNTDRHSIKKNLIGALLFDSGETAE |
| 1247 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | iAKSEQEIGKATAKYFFYSNIMNFFKTEITL |
| | | ANGEIRKRPLIETNGETGEIVWDKGRDFAT |
| | | VRKVLSMPQVNIVKKTEVQTGGFSKESILP |
| | | KRNSDKLIARKKDWDPKKYGGFDSPTVAYS |
| | | VLVVAKVEKGKSKKLKSVKELLGITIMERS |
| | | SFEKNPIDFLEAKGYKEVKKDLIIKLPKYS |
| | | LFELENGRKRMLASAGELQKGNELALPSKY |
| | | VNFLYLASHYEKLKGGSSGSETPGTSESAT |
| | | PESSGSEVEFSHEYWMRHALTLAKRARDER |
| | | EVPVGAVLVLNNRVIGEGWNRAIGLHDPTA |
| | | HAEIMALRQGGLVMQNYRLIDATLYVTFEP |
| | | CVMC |
| | | AGAMIHSRIGRWFGVRNAKTGAAGSLMDVL |
| | | HYPGMNHRVEITEGILADECAALLCYFFRM |
| | | PRQVFNAQKKAQSSTDSPEDNEQKQLFVEQ |
| | | HKHYLDEIIEQISEFSKRVILADANLDKVL |
| | | SAYNKHRDKPIREQAENIIHLFTLTNLGAP |
| | | AAFKYFDTTIDRKRYTSTKEVLDATLIHQS |
| | | ITGLYETRIDLSQLGGD |
| 107 Cas9 | 1321 | MDKKYSIGLAIGTNSVGWAVITDEYKVPSK |
| TadAins | | KFKVLGNTDRHSIKKNLIGALLFDSGETAE |
| 1054 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | WGTALIKKYPKLESEFVYGDYKVYDVRKMI |
| | | AKSEQEIGKATAKYFFYSNIMNFFKTEITL |
| | | ANGSSGSETPGTSESATPESSGSEVEFSHE |
| | | YWMRHALTLAKRARDEREVPVGAVLVLNNR |
| | | VIGEGWNRAIGLHDPTAHAEIMALRQGGLV |
| | | MQNYRLIDATLYVTFEPCVMCAGAMIHSRI |
| | | GRVVFGVRNAKTGAAGSLMDVLHYPGMNHR |
| | | VEITEGILADECAALLCYFFRMPRQVFNAQ |
| | | KKAQSSTDGEIRKRPLIETNGETGEIVWDK |
| | | GRDFATVRKVLSMPQVNIVKKTEVQTGGFS |
| | | KESILPKRNSDKLIARKKDWDPKKYGGFDS |
| | | PTVAYSVLVVAKVEKGKSKKLKSVKELLGI |
| | | TIMERSSFEKNPIDFLEAKGYKEVKKDLII |
| | | KLPKYSLFELENGRKRMLASAGELQKGNEL |
| | | ALPSKYVNFLYLASHYEKLKGSPEDNEQKQ |
| | | LFVEQHKHYLDEIIEQISEFSKRVILADAN |
| | | LDKVLSAYNKHRDKPIREQAENIIHLFTLT |
| | | NLGAPAAFKYFDTTIDRKRYTSTKEVLDAT |
| | | LIHQSITGLYETRIDLSQLGGD |
| 108 Cas9 | 1322 | MDKKYSIGLAIGTNSVGWAVITDEYKVPSK |
| TadAins | | KFKVLGNTDRHSIKKNLIGALLFDSGETAE |
| 1026 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AWGTALIKKYPKLESEFVYGDYKVYDVRKM |
| | | iAKSEGSSGSETPGTSESATPESSGSEVEFS |
| | | HEYWMRHALTLAKRARDEREVPVGAVLVLN |
| | | NRVIGEGWNRAIGLHDPTAHAEIMALRQGG |
| | | LVMQNYRLIDATLYVTFEPCVMCAGAMIHS |
| | | RIGRVVFGVRNAKTGAAGSLMDVLHYPGMN |
| | | HRVEITEGILADECAALLCYFFRMPRQVFN |
| | | AQKKAQSSTDQEIGKATAKYFFYSNIMNFF |
| | | KTEITLANGEIRKRPLIETNGETGEIVWDK |
| | | GRDFATVRKVLSMPQVNIVKKTEVQTGGFS |
| | | KESILPKRNSDKLIARKKDWDPKKYGGFDS |
| | | PTVAYSVLWAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGD |
| 109 Cas9 | 1323 | MDKKYSIGLAIGTNSVGWAVITDEYKVPSK |
| TadAins | | KFKVLGNTDRHSIKKNLIGALLFDSGETAE |
| 768 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQGSSGSETPGTSE |
| | | SATPESSGSEVEFSHEYWMRHALTLAKRAR |
| | | DEREVPVGAVLVLNNRVIGEGWNRAIGLHD |
| | | PTAHAEIMALRQGGLVMQNYRLIDATLYVT |
| | | FEPCVMCAGAMIHSRIGRWFGVRNAKTGAA |
| | | GSLMDVLHYPGMNHRVEITEGILADECAAL |
| | | LCYFFRMPRTTQKGQKNSRERMKRIEEGIK |
| | | ELGSQILKEHPVENTQLQNEKLYLYYLQNG |
| | | RDMYVDQELDINRLSDYDVDHIVPQSFLK |
| | | DDSIDNKVLTRSDKNRGKSDNVP |
| | | SEEWKKMKNYWRQLLNAKLITQRKFDNLTK |
| | | AERGGLSELDKAGFIKRQLVETRQITKHVA |
| | | QILDSRMNTKYDENDKLIREVKVITLKSKL |
| | | VSDFRKDFQFYKVREINNYHHAHDAYLNAVV |
| | | GTALIKKYPKLESEFVYGDYKVYDVRKMIA |
| | | KSEQEIGKATAKYFFYSNIMNFFKTEITLA |
| | | NGEIRKRPLIETNGETGEIVWDKGRDFATV |
| | | RKVLSMPQVNIVKKTEVQTGGFSKESILPK |
| | | RNSDKLIARKKDWDPKKYGGFDSPTVAYSV |
| | | LVVAKVEKGKSKKLKSVKELLGITIMERSS |
| | | FEKNPIDFLEAK |
| | | GYKEVKKDLIIKLPKYSLFELENGRKRMLA |
| | | SAGELQKGNELALPSKYVNFLYLASHYEKL |
| | | KGSPEDNEQKQLFVEQHKHYLDEIIEQISE |
| | | FSKRVILADANLDKVLSAYNKHRDKPIREQ |
| | | AENIIHLFTLTNLGAPAAFKYFDTTIDRKR |
| | | YTSTKEVLDATLIHQSITGLYETRIDLSQL |
| | | GGD |
| 110.1 Cas9 | 1324 | MDKKYSIGLAIGTNSVGWAVITDEYKVPSK |
| TadAins 1250 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLAISAGELQKGNELALP |
| | | SKYVNFLYLASHYEKLKGSPGSSGSETPGT |
| | | SESATPESSGSEVEFSHEYWMRHALTLAKR |
| | | ARDEREVPVGAVLVLNNRVIGEGWNRAIGL |
| | | HDPTAHAEIMALRQGGLVMQNYRLIDATLY |
| | | VTFEPCVMCAGAMIHSRIGRVVFGVRNAKT |
| | | GAAGSLMDVLHYPGMNHRVEITEGILADEC |
| | | AALLCYFFRMPREDNEQKQLFVEQHKHYLD |
| | | EIIEQISEFSKRVILADANLDKVLSAYNKH |
| | | RDKPIREQAENIIHLFTLTNLGAPAAFKYF |
| | | DTTIDRKRYTSTKEVLDATLIHQSITGLYE |
| | | TRIDLSQLGGD |
| 110.2 Cas9 | 1325 | MDKKYSIGLAIGTNSVGWAVITDEYKVPSK |
| TadAins 1250 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPGSSGSSGSETP |
| | | GTSESATPESSGSEVEFSHEYWMRHALTLA |
| | | KRARDEREVPVGAVLVLNNRVIGEGWNRAI |
| | | GLHDPTAHAEIMALRQGGLVMQNYRLIDAT |
| | | LYVTFEPCVMCAGAMIHSRIGRVVFGVRNA |
| | | KTGAAGSLMDVLHYPGMNHRVEITEGILAD |
| | | ECAALLCYFFRMPREDNEQKQLFVEQHKHY |
| | | LDEIIEQISEFSKRVILADANLDKVLSAYN |
| | | KHRDKPIREQAENIIHLFTLTNLGAPAAFK |
| | | YFDTTIDRKRYTSTKEVLDATLIHQSITGL |
| | | YETRIDLSQLGGD |
| 110.3 Cas9 | 1326 | MDKKYSIGLAIGTNSVGWAVITDEYKVPSK |
| TadAins 1250 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSPGSSGSSGSETPG |
| | | TSESATPESGSSSGSEVEFSHEYWMRHALT |
| | | LAKRARDEREVPVGAVLVLNNRVIGEGWNR |
| | | AIGLHDPTAHAEIMALRQGGLVMQNYRLID |
| | | ATLYVTFEPCVMCAGAMIHSRIGRVVFGVR |
| | | NAKTGAAGSLMDVLHYPGMNHRVEITEGIL |
| | | ADECAALLCYFFRMPREDNEQKQLFVEQHK |
| | | HYLDEIIEQISEFSKRVILADANLDKVLSA |
| | | YNKHRDKPIREQAENIIHLFTLTNLGAPAA |
| | | FKYFDTTIDRKRYTSTKEVLDATLIHQSIT |
| | | GLYETRIDLSQLGGD |
| 110.4 Cas9 | 1327 | MDKKYSIGLAIGTNSVGWAVITDEYKVPSK |
| TadAins 1250 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMU\SAGELQKGNELALPS |
| | | KYVNFLYLASHYEKLKGSPGSSGSSGSETP |
| | | GTSESATPESGSSSGSEVEFSHEYWMRHAL |
| | | TLAKRARDEREVPVGAVLVLNNRVIGEGWN |
| | | RAIGLHDPTAHAEIMALRQGGLVMQNYRLI |
| | | DATLYVTFEPCVMCAGAMIHSRIGRVVFGV |
| | | RNAKTGAAGSLMDVLHYPGMNHRVEITEGI |
| | | LADECAALLCYFFRMRREDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| 110.5 Cas9 | 1328 | MDKKYSIGLAIGTNSVGWAVITDEYKVPSK |
| TadAins 1249 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSGSSGSSGSETPGT |
| | | SESATPESGSSSGSEVEFSHEYWMRHALTL |
| | | AKRARDEREVPVGAVLVLNNRVIGEGWNRA |
| | | IGLHDPTAHAEIMALRQGGLVMQNYRLIDA |
| | | TLYVTFEPCVMCAGAMIHSRIGRVVFGVRN |
| | | AKTGAAGSLMDVLHYPGMNHRVEITEGILA |
| | | DECAALLCYFFRMRRPEDNEQKQLFVEQHK |
| | | HYLDEIIEQISEFSKRVILADANLDKVLSA |
| | | YNKHRDKPIREQAENIIHLFTLTNLGAPAA |
| | | FKYFDTTIDRKRYTSTKEVLDATLIHQSIT |
| | | GLYETRIDLSQLGGD |
| 110.5 Cas9 | 1329 | MDKKYSIGLAIGTNSVGWAVITDEYKVPSK |
| TadAins | | KFKVLGNTDRHSIKKNLIGALLFDSGETAE |
| delta 59- | | ATRLKRTARRRYTRRKNRICYLQEIFSNEM |
| 66 1250 | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPGSSGSSGSETP |
| | | GTSESATPESGSSGSEVEFSHEYWMRHALT |
| | | LAKRARDEREVPVGAVLVLNNRVIGEGWNR |
| | | AHAEIMALRQGGLVMQNYRLIDATLYVTFE |
| | | PCVMCAGAMIHSRIGRWFGVRNAKTGAAGS |
| | | LMDVLHYPGMNHRVEITEGILADECAALLC |
| | | YFFRMPRQVFNAQKKAQSSTDEDNEQKQLF |
| | | VEQHKHYLDEIIEQISEFSKRVILADANLD |
| | | KVLSAYNKHRDKPIREQAENIIHLFTLTNL |
| | | GAPAAFKYFDTTIDRKRYTSTKEVLDATLI |
| | | HQSITGLYETRIDLSQLGGD |
| 110.6 Cas9 | 1330 | MDKKYSIGLAIGTNSVGWAVITDEYKVPSK |
| TadAins 1251 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEGSSGSSGSET |
| | | PGTSESATPESGSSSGSEVEFSHEYWMRHA |
| | | LTLAKRARDEREVPVGAVLVLNNRVIGEGW |
| | | NRAIGLHDPTAHAEIMALRQGGLVMQNYRL |
| | | IDATLYVTFEPCVMCAGAMIHSRIGRVVFG |
| | | VRNAKTGAAGSLMDVLHYPGMNHRVEITEG |
| | | ILADECAALLCYFFRMRRDNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| 110.7 Cas9 | 1331 | MDKKYSIGLAIGTNSVGWAVITDEYKVPSK |
| TadAins 1252 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDGSSGSSGSE |
| | | TPGTSESATPESGSSSGSEVEFSHEYWMRH |
| | | ALTLAKRARDEREVPVGAVLVLNNRVIGEG |
| | | WNRAIGLHDPTAHAEIMALRQGGLVMQNYR |
| | | LIDATLYVTFEPCVMCAGAMIHSRIGRVVF |
| | | GVRNAKTGAAGSLMDVLHYPGMNHRVEITE |
| | | GILADECAALLCYFFRMRRNEQKQLFVEQH |
| | | KHYLDEIIEQISEFSKRVILADANLDKVLS |
| | | AYNKHRDKPIREQAENIIHLFTLTNLGAPA |
| | | AFKYFDTTIDRKRYTSTKEVLDATLIHQSI |
| | | TGLYETRIDLSQLGGD |
| 110.8 Cas9 | 1332 | MDKKYSIGLAIGTNSVGWAVITDEYKVPSK |
| TadAins | | KFKVLGNTDRHSIKKNLIGALLFDSGETAE |
| delta 59-66 | | ATRLKRTARRRYTRRKNRICYLQEIFSNEM |
| C-truncate | | AKVDDSFFHRLEESFLVEEDKKHERHPIFG |
| 1250 | | NIVDEVAYHEKYPTIYHLRKKLVDSTDKAD |
| polypeptide | | LRLIYLALAHMIKFRGHFLIEGDLNPDNSD |
| sequence | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEKNPIDFLEAKGYKEVKKDLIIKLPKY |
| | | SLFELENGRKRMLASAGELQKGNELALPSK |
| | | YVNFLYLASHYEKLKGSPGSSGSETPGTSE |
| | | SATPESSGSEVEFSHEYWMRHALTLAKRAR |
| | | DEREVPVGAVLVLNNRVIGEGWNRAHAEIM |
| | | ALRQGGLVMQNYRLIDATLYVTFEPCVMCA |
| | | GAMIHSRIGRVVFGVRNAKTGAAGSLMDVL |
| | | HYPGMNHRVEITEGILADECAALLCYFFRM |
| | | PRQEDNEQKQLFVEQHKHYLDEIIEQISEF |
| | | SKRVILADANLDKVLSAYNKHRDKPIREQA |
| | | ENIIHLFTLTNLGAPAAFKYFDTTIDRKRY |
| | | TSTKEVLDATLIHQSITGLYETRIDLSQLG |
| | | GD |
| 111.1 Cas9 | 1333 | MDKKYSIGLAIGTNSVGWAVITDEYKVPSK |
| TadAins 997 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRVVFGVRNAKTGAAGSLMDV |
| | | LHYPGMNHRVEITEGILADECAALLCYFFR |
| | | MPRQVFNAQKKAQSSTDGSSGSETPGTSES |
| | | ATPESSGIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDNEQKQLFVE |
| | | QHKHYLDEIIEQISEFSKRVILADANLDKV |
| | | LSAYNKHRDKPIREQAENIIHLFTLTNLGA |
| | | PAAFKYFDTTIDRKRYTSTKEVLDATLIHQ |
| | | SITGLYETRIDLSQLGGD |
| 111.2 Cas9 | 1334 | MDKKYSIGLAIGTNSVGWAVITDEYKVPSK |
| TadAins 997 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEWKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRWFGVRNAKTGAAGSLMDVL |
| | | HYPGMNHRVEITEGILADECAALLCYFFRM |
| | | PRQVFNAQKKAQSSTDGSSGSSGSETPGTS |
| | | ESATPESSGGSSIKKYPKLESEFVYGDYKV |
| | | YDVRKMIAKSEQEIGKATAKYFFYSNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLWAKVEKGKSKKLKSVKELLGI |
| | | TIMERSSFEKNPIDFLEAKGYKEVKKDLII |
| | | KLPKYSLFELENGRKRMLASAGELQKGNEL |
| | | ALPSKYVNFLYLASHYEKLKGSPEDNEQKQ |
| | | LFVEQHKHYLDEIIEQISEFSKRVILADAN |
| | | LDKVLSAYNKHRDKPIREQAENIIHLFTLT |
| | | NLGAPAAFKYFDTTIDRKRYTSTKEVLDAT |
| | | LIHQSITGLYETRIDLSQLGGD |
| 112 delta | 1335 | MDKKYSIGLAIGTNSVGWAVITDEYKVPSK |
| HNH | | KFKVLGNTDRHSIKKNLIGALLFDSGETAE |
| TadA | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSEVEFSHEYWMRHALTLA |
| | | KRARDEREVPVGAVLVLNNRVIGEGWNRAI |
| | | GLHDPTAHAEIMALRQGGLVMQNYRLIDAT |
| | | LYVTFEPCVMCAGAMIHSRIGRVVFGVRNA |
| | | KTGAAGSLMDVLHYPGMNHRVEITEGILAD |
| | | ECAALLCYFFRMPRQVFNAQKKAQSSTDGG |
| | | LSELDKAGFIKRQLVETRQITKHVAQILDS |
| | | RMNTKYDENDKLIREVKVITLKSKLVSDFR |
| | | KDFQFYKVREINNYHHAHDAYLNAVVGTAL |
| | | IKKYPKLESEFVYGDYKVYDVRKMIAKSEQ |
| | | EIGKATAKYFFYSNIMNFFKTEITLANGEI |
| | | RKRPLIETNGETGEIVWDKGRDFATVRKVL |
| | | SMPQVNIVKKTEVQTGGFSKESILPKRNSD |
| | | KLIARKKDWDPKKYGGFDSPTVAYSVLVVA |
| | | KVEKGKSKKLKSVKELLGITIMERSSFEKN |
| | | PIDFLEAKGYKEVKKDLIIKLPKYSLFELE |
| | | NGRKRMLASAGELQKGNELALPSKYVNFLY |
| | | LASHYEKLKGSPEDNEQKQLFVEQHKHYLD |
| | | EIIEQISEFSKRVILADANLDKVLSAYNKH |
| | | RDKPIREQAENIIHLFTLTNLGAPAAFKYF |
| | | DTTIDRKRYTSTKEVLDATLIHQSITGLYE |
| | | TRIDLSQLGGD |
| 113 N-term | 1336 | MSEVEFSHEYWMRHALTLAKRARDEREVPV |
| single | | GAVLVLNNRVIGEGWNRAIGLHDPTAHAEI |
| TadA helix | | MALRQGGLVMQNYRLIDATLYVTFEPCVMC |
| trunc | | AGAMIHSRIGRVVFGVRNAKTGAAGSLMDV |
| 165-end | | LHYPGMNHRVEITEGILADECAALLCYFFR |
| polypeptide | | MPRSGGSSGGSSGSETPGTSESATPESSGG |
| sequence | | SSGGSDKKYSIGLAIGTNSVGWAVITDEYK |
| | | VPSKKFKVLGNTDRHSIKKNLIGALLFDSG |
| | | ETAEATRLKRTARRRYTRRKNRICYLQEIF |
| | | SNEMAKVDDSFFHRLEESFLVEEDKKHERH |
| | | PIFGNIVDEVAYHEKYPTIYHLRKKLVDST |
| | | DKADLRLIYLALAHMIKFRGHFLIEGDLNP |
| | | DNSDVDKLFIQLVQTYNQLFEENPINASGV |
| | | DAKAILSARLSKSRRLENLIAQLPGEKKNG |
| | | LFGNLIALSLGLTPNFKSNFDLAEDAKLQL |
| | | SKDTYDDDLDNLLAQIGDQYADLFLAAKNL |
| | | SDAILLSDILRVNTEITKAPLSASMIKRYD |
| | | EHHQDLTLLKALVRQQLPEKYKEIFFDQSK |
| | | NGYAGYIDGGASQEEFYKFIKPILEKMDGT |
| | | EELLVKLNREDLLRKQRTFDNGSIPHQIHL |
| | | GELHAILRRQEDFYPFLKDNREKIEKILTF |
| | | RIPYYVGPLARGNSRFAWMTRKSEETITPW |
| | | NFEEVVDKGASAQSFIERMTNFDKNLPNEK |
| | | VLPKHSLLYEYFTVYNELTKVKYVTEGMRK |
| | | PAFLSGEQKKAIVDLLFKTNRKVTVKQLKE |
| | | DYFKKIECFDSVEISGVEDRFNASLGTYHD |
| | | LLKIIKDKDFLDNEENEDILEDIVLTLTLF |
| | | EDREMIEERLKTYAHLFDDKVMKQLKRRRY |
| | | TGWGRLSRKLINGIRDKQSGKTILDFLKSD |
| | | GFANRNFMQLIHDDSLTFKEDIQKAQVSGQ |
| | | GDSLHEHIANLAGSPAIKKGILQTVKVVDE |
| | | LVKVMGRHKPENIVIEMARENQTTQKGQKN |
| | | SRERMKRIEEGIKELGSQILKEHPVENTQL |
| | | QNEKLYLYYLQNGRDMYVDQELDINRLSDY |
| | | DVDHIVPQSFLKDDSIDNKVLTRSDKNRGK |
| | | SDNVPSEEWKKMKNYWRQLLNAKLITQRKF |
| | | DNLTKAERGGLSELDKAGFIKRQLVETRQI |
| | | TKHVAQILDSRMNTKYDENDKLIREVKVIT |
| | | LKSKLVSDFRKDFQFYKVREINNYHHAHDA |
| | | YLNAVVGTALIKKYPKLESEFVYGDYKVYD |
| | | VRKMIAKSEQEIGKATAKYFFYSNIMNFFK |
| | | TEITLANGEIRKRPLIETNGETGEIVWDKG |
| | | RDFATVRKVLSMPQVNIVKKTEVQTGGFSK |
| | | ESILPKRNSDKLIARKKDWDPKKYGGFDSP |
| | | TVAYSVLWAKVEKGKSKKLKSVKELLGITI |
| | | MERSSFEKNPIDFLEAKGYKEVKKDLIIKL |
| | | PKYSLFELENGRKRMLASAGELQKGNELAL |
| | | PSKYVNFLYLASHYEKLKGSPEDNEQKQLF |
| | | VEQHKHYLDEIIEQISEFSKRVILADANLD |
| | | KVLSAYNKHRDKPIREQAENIIHLFTLTNL |
| | | GAPAAFKYFDTTIDRKRYTSTKEVLDATLI |
| | | HQSITGLYETRIDLSQLGGD |
| 114 N-term | 1337 | MSEVEFSHEYWMRHALTLAKRARDEREVPV |
| single | | GAVLVLNNRVIGEGWNRTAHAEIMALRQGG |
| TadA helix | | LVMQNYRLIDATLYVTFEPCVMCAGAMIHS |
| trunc | | RIGRWFGVRNAKTGAAGSLMDVLHYPGMNH |
| 165-end | | RVEITEGILADECAALLCYFFRMPRSGGSS |
| delta | | GGSSGSETPGTSESATPESSGGSSGGSDKK |
| 59-65 | | YSIGLAIGTNSVGWAVITDEYKVPSKKFKV |
| polypeptide | | LGNTDRHSIKKNLIGALLFDSGETAEATRL |
| sequence | | KRTARRRYTRRKNRICYLQEIFSNEMAKVD |
| | | DSFFHRLEESFLVEEDKKHERHPIFGNIVD |
| | | EVAYHEKYPTIYHLRKKLVDSTDKADLRLI |
| | | YLALAHMIKFRGHFLIEGDLNPDNSDVDKL |
| | | FIQLVQTYNQLFEENPINASGVDAKAILSA |
| | | RLSKSRRLENLIAQLPGEKKNGLFGNLIAL |
| | | SLGLTPNFKSNFDLAEDAKLQLSKDTYDDD |
| | | LDNLLAQIGDQYADLFLAAKNLSDAILLSD |
| | | ILRVNTEITKAPLSASMIKRYDEHHQDLTL |
| | | LKALVRQQLPEKYKEIFFDQSKNGYAGYID |
| | | GGASQEEFYKFIKPILEKMDGTEELLVKLN |
| | | REDLLRKQRTFDNGSIPHQIHLGELHAILR |
| | | RQEDFYPFLKDNREKIEKILTFRIPYYVGP |
| | | LARGNSRFAWMTRKSEETITPWNFEEVVDK |
| | | GASAQSFIERMTNFDKNLPNEKVLPKHSLL |
| | | YEYFTVYNELTKVKYVTEGMRKPAFLSGEQ |
| | | KKAIVDLLFKTNRKVTVKQLKEDYFKKIEC |
| | | FDSVEISGVEDRFNASLGTYHDLLKIIKDK |
| | | DFLDNEENEDILEDIVLTLTLFEDREMIEE |
| | | RLKTYAHLFDDKVMKQLKRRRYTGWGRLSR |
| | | KLINGIRDKQSGKTILDFLKSDGFANRNFM |
| | | QLIHDDSLTFKEDIQKAQVSGQGDSLHEHI |
| | | ANLAGSPAIKKGILQTVKWDELVKVMGRHK |
| | | PENIVIEMARENQTTQKGQKNSRERMKRIE |
| | | EGIKELGSQILKEHPVENTQLQNEKLYLYY |
| | | LQNGRDMYVDQELDINRLSDYDVDHIVPQS |
| | | FLKDDSIDNKVLTRSDKNRGKSDNVPSEEW |
| | | KKMKNYWRQLLNAKLITQRKFDNLTKAERG |
| | | GLSELDKAGFIKRQLVETRQITKHVAQILD |
| | | SRMNTKYDENDKLIREVKVITLKSKLVSDF |
| | | RKDFQFYKVREINNYHHAHDAYLNAVVGTA |
| | | LIKKYPKLESEFVYGDYKVYDVRKMIAKSE |
| | | QEIGKATAKYFFYSNIMNFFKTEITLANGE |
| | | IRKRPLIETNGETGEIVWDKGRDFATVRKV |
| | | LSMPQVNIVKKTEVQTGGFSKESILPKRNS |
| | | DKLIARKKDWDPKKYGGFDSPTVAYSVLVV |
| | | AKVEKGKSKKLKSVKELLGITIMERSSFEK |
| | | NPIDFLEAKGYKEVKKDLIIKLPKYSLFEL |
| | | ENGRKRMLASAGELQKGNELALPSKYVNFL |
| | | YLASHYEKLKGSPEDNEQKQLFVEQHKHYL |
| | | DEIIEQISEFSKRVILADANLDKVLSAYNK |
| | | HRDKPIREQAENIIHLFTLTNLGAPAAFKY |
| | | FDTTIDRKRYTSTKEVLDATLIHQSITGLY |
| | | ETRIDLSQLGGD |
| 115.1 Cas9 | 1338 | MDKKYSIGLAIGTNSVGWAVITDEYKVPSK |
| TadAins1004 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLV |
| | | QTYNQLFEENPINASGVDAKAILSARLSKS |
| | | RRLENLIAQLPGEKKNGLFGNLIALSLGLT |
| | | PNFKSNFDLAEDAKLQLSKDTYDDDLDNLL |
| | | AQIGDQYADLFLAAKNLSDAILLSDILRVN |
| | | TEITKAPLSASMIKRYDEHHQDLTLLKALV |
| | | RQQLPEKYKEIFFDQSKNGYAGYIDGGASQ |
| | | EEFYKFIKPILEKMDGTEELLVKLNREDLL |
| | | RKQRTFDNGSIPHQIHLGELHAILRRQEDF |
| | | YPFLKDNREKIEKILTFRIPYYVGPLARGN |
| | | SRFAWMTRKSEETITPWNFEEVVDKGASAQ |
| | | SFIERMTNFDKNLPNEKVLPKHSLLYEYFT |
| | | VYNELTKVKYVTEGMRKPAFLSGEQKKAIV |
| | | DLLFKTNRKVTVKQLKEDYFKKIECFDSVE |
| | | ISGVEDRFNASLGTYHDLLKIIKDKDFLDN |
| | | EENEDILEDIVLTLTLFEDREMIEERLKTY |
| | | AHLFDDKVMKQLKRRRYTGWGRLSRKLING |
| | | IRDKQSGKTILDFLKSDGFANRNFMQLIHD |
| | | DSLTFKEDIQKAQVSGQGDSLHEHIANLAG |
| | | SPAIKKGILQTVKVVDELVKVMGRHKPENI |
| | | VIEMARENQTTQKGQKNSRERMKRIEEGIK |
| | | ELGSQILKEHPVENTQLQNEKLYLYYLQNG |
| | | RDMYVDQELDINRLSDYDVDHIVPQSFLKD |
| | | DSIDNKVLTRSDKNRGKSDNVPSEEVVKKM |
| | | KNYWRQLLNAKLITQRKFDNLTKAERGGLS |
| | | ELDKAGFIKRQLVETRQITKHVAQILDSRM |
| | | NTKYDENDKLIREVKVITLKSKLVSDFRKD |
| | | FQFYKVREINNYHHAHDAYLNAVVGTALIK |
| | | KYPKGSSGSETPGTSESATPESSGSEVEFS |
| | | HEYWMRHALTLAKRARDEREVPVGAVLVLN |
| | | NRVIGEGWNRAIGLHDPTAHAEIMALRQGG |
| | | LVMQNYRLIDATLYVTFEPCVMCAGAMIHS |
| | | RIGRVVFGVRNAKTGAAGSLMDVLHYPGMN |
| | | HRVEITEGILADECAALLCYFFRMPRQLES |
| | | EFVYGDYKVYDVRKMIAKSEQEIGKATAKY |
| | | FFYSNIMNFFKTEITLANGEIRKRPLIETN |
| | | GETGEIVWDKGRDFATVRKVLSMPQVNIVK |
| | | KTEVQTGGFSKESILPKRNSDKLIARKKDW |
| | | DPKKYGGFDSPTVAYSVLVVAKVEKGKSKK |
| | | LKSVKELLGITIMERSSFEKNPIDFLEAKG |
| | | YKEVKKDLIIKLPKYSLFELENGRKRMLAS |
| | | AGELQKGNELALPSKYVNFLYLASHYEKLK |
| | | GSPEDNEQKQLFVEQHKHYLDEIIEQISEF |
| | | SKRVILADANLDKVLSAYNKHRDKPIREQA |
| | | ENIIHLFTLTNLGAPAAFKYFDTTIDRKRY |
| | | TSTKEVLDATLIHQSITGLYETRIDLSQLG |
| | | GD |
| 115.2 Cas9 | 1339 | MDKKYSIGLMGTNSVGWAVITDEYKVPSKK |
| TadAins1005 | | FKVLGNTDRHSIKKNLIGALLFDSGETAEA |
| polypeptide | | TRLKRTARRRYTRRKNRICYLQEIFSNEMA |
| sequence | | KVDDSFFHRLEESFLVEEDKKHERHPIFGN |
| | | IVDEVAYHEKYPTIYHLRKKLVDSTDKADL |
| | | RLIYLALAHMIKFRGHFLIEGDLNPDNSDV |
| | | DKLFIQLVQTYNQLFEENPINASGVDAKAI |
| | | LSARLSKSRRLENLIAQLPGEKKNGLFGNL |
| | | IALSLGLTPNFKSNFDLAEDAKLQLSKDTY |
| | | DDDLDNLLAQIGDQYADLFLAAKNLSDAIL |
| | | LSDILRVNTEITKAPLSASMIKRYDEHHQD |
| | | LTLLKALVRQQLPEKYKEIFFDQSKNGYAG |
| | | YIDGGASQEEFYKFIKPILEKMDGTEELLV |
| | | KLNREDLLRKQRTFDNGSIPHQIHLGELHA |
| | | ILRRQEDFYPFLKDNREKIEKILTFRIPYY |
| | | VGPLARGNSRFAWMTRKSEETITPWNFEEV |
| | | VDKGASAQSFIERMTNFDKNLPNEKVLPKH |
| | | SLLYEYFTVYNELTKVKYVTEGMRKPAFLS |
| | | GEQKKAIVDLLFKTNRKVTVKQLKEDYFKK |
| | | IECFDSVEISGVEDRFNASLGTYHDLLKII |
| | | KDKDFLDNEENEDILEDIVLTLTLFEDREM |
| | | IEERLKTYAHLFDDKVMKQLKRRRYTGWGR |
| | | LSRKLINGIRDKQSGKTILDFLKSDGFANR |
| | | NFMQLIHDDSLTFKEDIQKAQVSGQGDSLH |
| | | EHIANLAGSPAIKKGILQTVKVVDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLGSSGSETPGTSESATPE |
| | | SSGSEVEFSHEYWMRHALTLAKRARDEREV |
| | | PVGAVLVLNNRVIGEGWNRAIGLHDPTAHA |
| | | EIMALRQGGLVMQNYRLIDATLYVTFEPCV |
| | | MCAGAMIHSRIGRVVFGVRNAKTGAAGSLM |
| | | DVLHYPGMNHRVEITEGILADECAALLCYF |
| | | FRMPRQESEFVYGDYKVYDVRKMIAKSEQE |
| | | IGKATAKYFFYSNIMNFFKTEITLANGEIR |
| | | KRPLIETNGETGEIVWDKGRDFATVRKVLS |
| | | MPQVNIVKKTEVQTGGFSKESILPKRNSDK |
| | | LIARKKDWDPKKYGGFDSPTVAYSVLWAKV |
| | | EKGKSKKLKSVKELLGITIMERSSFEKNPI |
| | | DFLEAKGYKEVKKDLIIKLPKYSLFELENG |
| | | RKRMLASAGELQKGNELALPSKYVNFLYLA |
| | | SHYEKLKGSPEDNEQKQLFVEQHKHYLDEI |
| | | IEQISEFSKRVILADANLDKVLSAYNKHRD |
| | | KPIREQAENIIHLFTLTNLGAPAAFKYFDT |
| | | TIDRKRYTSTKEVLDATLIHQSITGLYETR |
| | | IDLSQLGGD |
| 115.3 Cas9 | 1340 | MDKKYSIGLAIGTNSVGWAVITDEYKVPSK |
| TadAins1006 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLEGSSGSETPGTSESAT |
| | | PESSGSEVEFSHEYWMRHALTLAKRARDER |
| | | EVPVGAVLVLNNRVIGEGWNRAIGLHDPTA |
| | | HAEIMALRQGGLVMQNYRLIDATLYVTFEP |
| | | CVMCAGAMIHSRIGRVVFGVRNAKTGAAGS |
| | | LMDVLHYPGMNHRVEITEGILADECAALLC |
| | | YFFRMPRQSEFVYGDYKVYDVRKMIAKSEQ |
| | | EIGKATAKYFFYSNIMNFFKTEITLANGEI |
| | | RKRPLIETNGETGEIVWDKGRDFATVRKVL |
| | | SMPQVNIVKKTEVQTGGFSKESILPKRNSD |
| | | KLIARKKDWDPKKYGGFDSPTVAYSVLWAK |
| | | VEKGKSKKLKSVKELLGITIMERSSFEKNP |
| | | IDFLEAKGYKEVKKDLIIKLPKYSLFELEN |
| | | GRKRMLASAGELQKGNELALPSKYVNFLYL |
| | | ASHYEKLKGSPEDNEQKQLFVEQHKHYLDE |
| | | IIEQISEFSKRVILADANLDKVLSAYNKHR |
| | | DKPIREQAENIIHLFTLTNLGAPAAFKYFD |
| | | TTIDRKRYTSTKEVLDATLIHQSITGLYET |
| | | RIDLSQLGGD |
| 115.4 Cas9 | 1341 | MDKKYSIGLAIGTNSVGWAVITDEYKVPSK |
| TadAins1007 | | KFKVLGNTDRHSIKKNLIGALLFDSGETAE |
| polypeptide | | ATRLKRTARRRYTRRKNRICYLQEIFSNEM |
| sequence | | AKVDDSFFHRLEESFLVEEDKKHERHPIFG |
| | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESGSSGSETPGTSESA |
| | | TPESSGSEVEFSHEYWMRHALTLAKRARDE |
| | | REVPVGAVLVLNNRVIGEGWNRAIGLHDP |
| | | TAHAEIMALRQGGLVMQNYRLIDATLYVTF |
| | | EPCVMCAGAMIHSRIGRVVFGVRNAKTGAA |
| | | GSLMDVLHYPGMNHRVEITEGILADECAAL |
| | | LCYFFRMPRQEFVYGDYKVYDVRKMIAKSE |
| | | QEIGKATAKYFFYSNIMNFFKTEITLANGE |
| | | IRKRPLIETNGETGEIVWDKGRDFATVRKV |
| | | LSMPQVNIVKKTEVQTGGFSKESILPKRNS |
| | | DKLIARKKDWDPKKYGGFDSPTVAYSVLVV |
| | | AKVEKGKSKKLKSVKELLGITIMERSSFEK |
| | | NPIDFLEAKGYKEVKKDLIIKLPKYSLFEL |
| | | ENGRKRMLASAGELQKGNELALPSKYVNFL |
| | | YLASHYEKLKGSPEDNEQKQLFVEQHKHYL |
| | | DEIIEQISEFSKRVILADANLDKVLSAYNK |
| | | HRDKPIREQAENIIHLFTLTNLGAPAAFKY |
| | | FDTTIDRKRYTSTKEVLDATLIHQSITGLY |
| | | ETRIDLSQLGGD |
| 116.1 Cas9 | 1342 | MDKKYSIGLAIGTNSVGWAVITDEYKVPSK |
| TadAins | | KFKVLGNTDRHSIKKNLIGALLFDSGETAE |
| C-term | | ATRLKRTARRRYTRRKNRICYLQEIFSNEM |
| truncate2 | | AKVDDSFFHRLEESFLVEEDKKHERHPIFG |
| 792 | | NIVDEVAYHEKYPTIYHLRKKLVDSTDKAD |
| polypeptide | | LRLIYLALAHMIKFRGHFLIEGDLNPDNSD |
| sequence | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGGSSGSETPGTSESATPES |
| | | SGSEVEFSHEYWMRHALTLAKRARDEREVP |
| | | VGAVLVLNNRVIGEGWNRAIGLHDPTAHAE |
| | | IMALRQGGLVMQNYRLIDATLYVTFEPCVM |
| | | CAGAMIHSRIGRVVFGVRNAKTGAAGSLMD |
| | | VLHYPGMNHRVEITEGILADECAALLCYFF |
| | | RMPRQSQILKEHPVENTQLQNEKLYLYYLQ |
| | | NGRDMYVDQELDINRLSDYDVDHIVPQSFL |
| | | KDDSIDNKVLTRSDKNRGKSDNVPSEEWKK |
| | | MKNYWRQLLNAKLITQRKFDNLTKAERGGL |
| | | SELDKAGFIKRQLVETRQITKHVAQILDSR |
| | | MNTKYDENDKLIREVKVITLKSKLVSDFRK |
| | | DFQFYKVREINNYHHAHDAYLNAVVGTALI |
| | | KKYPKLESEFVYGDYKVYDVRKMIAKSEQE |
| | | IGKATAKYFFYSNIMNFFKTEITLANGEIR |
| | | KRPLIETNGETGEIVWDKGRDFATVRKVLS |
| | | MPQVNIVKKTEVQTGGFSKESILPKRNSDK |
| | | LIARKKDWDPKKYGGFDSPTVAYSVLVVAK |
| | | VEKGKSKKLKSVKELLGITIMERSSFEKNP |
| | | IDFLEAKGYKEVKKDLIIKLPKYSLFELEN |
| | | GRKRMLASAGELQKGNELALPSKYVNFLYL |
| | | ASHYEKLKGSPEDNEQKQLFVEQHKHYLDE |
| | | IIEQISEFSKRVILADANLDKVLSAYNKHR |
| | | DKPIREQAENIIHLFTLTNLGAPAAFKYFD |
| | | TTIDRKRYTSTKEVLDATLIHQSITGLYET |
| | | RIDLSQLGGD |
| 116.2 Cas9 | 1343 | MDKKYSIGLAIGTNSVGWAVITDEYKVPSK |
| TadAins | | KFKVLGNTDRHSIKKNLIGALLFDSGETAE |
| C-term | | ATRLKRTARRRYTRRKNRICYLQEIFSNEM |
| truncate2 | | AKVDDSFFHRLEESFLVEEDKKHERHPIFG |
| 791 | | NIVDEVAYHEKYPTIYHLRKKLVDSTDKAD |
| polypeptide | | LRLIYLALAHMIKFRGHFLIEGDLNPDNSD |
| sequence | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSSGSETPGTSESATPESS |
| | | GSEVEFSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRWFGVRNAKTGAAGSLMDVL |
| | | HYPGMNHRVEITEGILADECAALLCYFFRM |
| | | PRQGSQILKEHPVENTQLQNEKLYLYYLQN |
| | | GRDMYVDQELDINRLSDYDVDHIVPQSFLK |
| | | DDSIDNKVLTRSDKNRGKSDNVPSEEWKKM |
| | | KNYWRQLLNAKLITQRKFDNLTKAERGGLS |
| | | ELDKAGFIKRQLVETRQITKHVAQILDSRM |
| | | NTKYDENDKLIREVKVITLKSKLVSDFRKD |
| | | FQFYKVREINNYHHAHDAYLNAVVGTALIK |
| | | KYPKLESEFVYGDYKVYDVRKMIAKSEQEI |
| | | GKATAKYFFYSNIMNFFKTEITLANGEIRK |
| | | RPLIETNGETGEIVWDKGRDFATVRKVLSM |
| | | PQVNIVKKTEVQTGGFSKESILPKRNSDKL |
| | | IARKKDWDPKKYGGFDSPTVAYSVLVVAKV |
| | | EKGKSKKLKSVKELLGITIMERSSFEKNPI |
| | | DFLEAKGYKEVKKDLIIKLPKYSLFELENG |
| | | RKRMLASAGELQKGNELALPSKYVNFLYLA |
| | | SHYEKLKGSPEDNEQKQLFVEQHKHYLDEI |
| | | IEQISEFSKRVILADANLDKVLSAYNKHRD |
| | | KPIREQAENIIHLFTLTNLGAPAAFKYFDT |
| | | TIDRKRYTSTKEVLDATLIHQSITGLYETR |
| | | IDLSQLGGD |
| 116.3 Cas9 | 1344 | MDKKYSIGLAIGTNSVGWAVITDEYKVPSK |
| TadAins | | KFKVLGNTDRHSIKKNLIGALLFDSGETAE |
| C-term | | ATRLKRTARRRYTRRKNRICYLQEIFSNEM |
| truncate2 | | AKVDDSFFHRLEESFLVEEDKKHERHPIFG |
| 790 | | NIVDEVAYHEKYPTIYHLRKKLVDSTDKAD |
| polypeptide | | LRLIYLALAHMIKFRGHFLIEGDLNPDNSD |
| sequence | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKEGSSGSETPGTSESATPESSG |
| | | SEVEFSHEYWMRHALTLAKRARDEREVPVG |
| | | AVLVLNNRVIGEGWNRAIGLHDPTAHAEIM |
| | | ALRQGGLVMQNYRLIDATLYVTFEPCVMCA |
| | | GAMIHSRIGRVVFGVRNAKTGAAGSLMDVL |
| | | HYPGMNHRVEITEGILADECAALLCYFFRM |
| | | PRQLGSQILKEHPVENTQLQNEKLYLYYLQ |
| | | NGRDMYVDQELDINRLSDYDVDHIVPQSFL |
| | | KDDSIDNKVLTRSDKNRGKSDNVPSEEWKK |
| | | MKNYWRQLLNAKLITQRKFDNLTKAERGGL |
| | | SELDKAGFIKRQLVETRQITKHVAQILDSR |
| | | MNTKYDENDKLIREVKVITLKSKLVSDFRK |
| | | DFQFYKVREINNYHHAHDAYLNAVVGTALI |
| | | KKYPKLESEFVYGDYKVYDVRKMIAKSEQE |
| | | IGKATAKYFFYSNIMNFFKTEITLANGEIR |
| | | KRPLIETNGETGEIVWDKGRDFATVRKVLS |
| | | MPQVNIVKKTEVQTGGFSKESILPKRNSDK |
| | | LIARKKDWDPKKYGGFDSPTVAYSVLVVAK |
| | | VEKGKSKKLKSVKELLGITIMERSSFEKNP |
| | | IDFLEAKGYKEVKKDLIIKLPKYSLFELEN |
| | | GRKRMLASAGELQKGNELALPSKYVNFLYL |
| | | ASHYEKLKGSPEDNEQKQLFVEQHKHYLDE |
| | | IIEQISEFSKRVILADANLDKVLSAYNKHR |
| | | DKPIREQAENIIHLFTLTNLGAPAAFKYFD |
| | | TTIDRKRYTSTKEVLDATLIHQSITGLYET |
| | | RIDLSQLGGD |
| 117 Cas9 | 1345 | MDKKYSIGLAIGTNSVGWAVITDEYKVPSK |
| delta | | KFKVLGNTDRHSIKKNLIGALLFDSGETAE |
| 1017-1069 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYSSGS |
| | | EVEFSHEYWMRHALTLAKRARDEREVPVGA |
| | | VLVLNNRVIGEGWNRAIGLHDPTAHAEIMA |
| | | LRQGGLVMQNYRLIDATLYVTFEPCVMCAG |
| | | AMIHSRIGRVVFGVRNAKTGAAGSLMDVLH |
| | | YPGMNHRVEITEGILADECAALLCYFFRMP |
| | | RQVFNAQKKAQSSTDGEIVWDKGRDFATVR |
| | | KVLSMPQVNIVKKTEVQTGGFSKESILPKR |
| | | NSDKLIARKKDWDPKKYGGFDSPTVAYSVL |
| | | WAKVEKGKSKKLKSVKELLGITIMERSSFE |
| | | KNPIDFLEAKGYKEVKKDLIIKLPKYSLFE |
| | | LENGRKRMLASAGELQKGNELALPSKYVNF |
| | | LYLASHYEKLKGSPEDNEQKQLFVEQHKHY |
| | | LDEIIEQISEFSKRVILADANLDKVLSAYN |
| | | KHRDKPIREQAENIIHLFTLTNLGAPAAFK |
| | | YFDTTIDRKRYTSTKEVLDATLIHQSITGL |
| | | YETRIDLSQLGGD |
| 118 Cas9 | 1346 | MDKKYSIGLAIGTNSVGWAVITDEYKVPSK |
| TadA- | | KFKVLGNTDRHSIKKNLIGALLFDSGETAE |
| CP116ins | | ATRLKRTARRRYTRRKNRICYLQEIFSNEM |
| 1067 | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNMNHRVEITEGILAD |
| | | ECAALLCYFFRMPRQVFNAQKKAQSSTDGS |
| | | SGSETPGTSESATPESSGSEVEFSHEYWMR |
| | | HALTLAKRARDEREVPVGAVLVLNNRVIGE |
| | | GWNRAIGLHDPTAHAEIMALRQGGLVMQNY |
| | | RLIDATLYVTFEPCVMCAGAMIHSRIGRVV |
| | | FGVRNAKTGAAGSLMDVLHYPGGETGEIVW |
| | | DKGRDFATVRKVLSMPQVNIVKKTEVQTGG |
| | | FSKESILPKRNSDKLIARKKDWDPKKYGGF |
| | | DSPTVAYSVLVVAKVEKGKSKKLKSVKELL |
| | | GITIMERSSFEKNPIDFLEAKGYKEVKKDL |
| | | IIKLPKYSLFELENGRKRMLASAGELQKGN |
| | | ELALPSKYVNFLYLASHYEKLKGSPEDNEQ |
| | | KQLFVEQHKHYLDEIIEQISEFSKRVILAD |
| | | ANLDKVLSAYNKHRDKPIREQAENIIHLFT |
| | | LTNLGAPAAFKYFDTTIDRKRYTSTKEVLD |
| | | ATLIHQSITGLYETRIDLSQLGGD |
| 119 Cas9 | 1347 | MDKKYSIGLAIGTNSVGWAVITDEYKVPSK |
| TadAins | | KFKVLGNTDRHSIKKNLIGALLFDSGETAE |
| 701 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSGSSGSETPGTSESATPESS |
| | | GSEVEFSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRVVFGVRNAKTGAAGSLMDV |
| | | LHYPGMNHRVEITEGILADECAALLCYFFR |
| | | MPRQVFNAQKKAQSSTDLTFKEDIQKAQVS |
| | | GQGDSLHEHIANLAGSPAIKKGILQTVKVV |
| | | DELVKVMGRHKPENIVIEMARENQTTQKGQ |
| | | KNSRERMKRIEEGIKELGSQILKEHPVENT |
| | | QLQNEKLYLYYLQNGRDMYVDQELDINRLS |
| | | DYDVDHIVPQSFLKDDSIDNKVLTRSDKNR |
| | | GKSDNVPSEEWKKMKNYWRQLLNAKLITQR |
| | | KFDNLTKAERGGLSELDKAGFIKRQLVETR |
| | | QITKHVAQILDSRMNTKYDENDKLIREVKV |
| | | ITLKSKLVSDFRKDFQFYKVREINNYHHAH |
| | | DAYLNAVVGTALIKKYPKLESEFVYGDYKV |
| | | YDVRKMIAKSEQEIGKATAKYFFYSNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLVVAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 120 Cas9 | 1348 | MDKKYSIGLAIGTNSVGWAVITDEYKVPSK |
| TadACP136 | | KFKVLGNTDRHSIKKNLIGALLFDSGETAE |
| ins | | ATRLKRTARRRYTRRKNRICYLQEIFSNEM |
| 1248 | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKWDELVKVM |
| | | GRHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFLKDDSIDNKVLTRSDKNRGKSDNVP |
| | | SEEVVKKMKNYWRQLLNAKLITQRKFDNLT |
| | | KAERGGLSELDKAGFIKRQLVETRQITKHV |
| | | AQILDSRMNTKYDENDKLIREVKVITLKSK |
| | | LVSDFRKDFQFYKVREINNYHHAHDAYLNA |
| | | VVGTALIKKYPKLESEFVYGDYKVYDVRKM |
| | | IAKSEQEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFDSPTVAY |
| | | SVLVVAKVEKGKSKKLKSVKELLGITIMER |
| | | SSFEK |
| | | NPIDFLEAKGYKEVKKDLIIKLPKYSLFEL |
| | | ENGRKRMLASAGELQKGNELALPSKYVNFL |
| | | YLASHYEKLKGSMNHRVEITEGILADECAA |
| | | LLCYFFRMPRQVFNAQKKAQSSTDGSSGSE |
| | | TPGTSESATPESSGSEVEFSHEYWMRHALT |
| | | LAKRARDEREVPVGAVLVLNNRVIGEGWNR |
| | | AIGLHDPTAHAEIMALRQGGLVMQNYRLID |
| | | ATLYVTFEPCVMCAGAMIHSRIGRWFGVRN |
| | | AKTGAAGSLMDVLHYPGPEDNEQKQLFVEQ |
| | | HKHYLDEIIEQISEFSKRVILADANLDKVL |
| | | SAYNKHRDKPIREQAENIIHLFTLTNLGAP |
| | | AAFKYFDTTIDRKRYTSTKEVLDATLIHQS |
| | | ITGLYETRIDLSQLGGD |
| 121 Cas9 | 1349 | MDKKYSIGLAIGTNSVGWAVITDEYKVPSK |
| TadACP136ins | | KFKVLGNTDRHSIKKNLIGALLFDSGETAE |
| 1052 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLAMNHRVEITEGILADECAALLCYFFRMP |
| | | RQVFNAQKKAQSSTDGSSGSETPGTSESAT |
| | | PESSGSEVEFSHEYWMRHALTLAKRARDER |
| | | EVPVGAVLVLNNRVIGEGWNRAIGLHDPTA |
| | | HAEIMALRQGGLVMQNYRLIDATLYVTFEP |
| | | CVMCAGAMIHSRIGRWFGVRNAKTGAAGSL |
| | | MDVLHYPGNGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLVVAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 122 Cas9 | 1350 | MDKKYSIGLAIGTNSVGWAVITDEYKVPSK |
| TadACP136 | | KFKVLGNTDRHSIKKNLIGALLFDSGETAE |
| ins | | ATRLKRTARRRYTRRKNRICYLQEIFSNEM |
| 1041 | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHK |
| | | PENIVIEMARENQTTQKGQKNSRERMKRIE |
| | | EGIKELGSQILKEHPVENTQLQNEKLYLYY |
| | | LQNGRDMYVDQELDINRLSDYDVDHIVPQS |
| | | FLKDDSIDNKVLTRSDKNRGKSDNVPSEEV |
| | | VKKMKNYWRQLLNAKLITQRKFDNLTKAER |
| | | GGLSELDKAGFIKRQLVETRQITKHVAQIL |
| | | DSRMNTKYDENDKLIREVKVITLKSKLVSD |
| | | FRKDFQFYKVREINNYHHAHDAYLNAVVGT |
| | | ALIKKYPKLESEFVYGDYKVYDVRKMIAKS |
| | | EQEIGKATAKYFFYSMNHRVEITEGILADE |
| | | CAALLCYFFRMPRQVFNAQKKAQSSTDGSS |
| | | GSETPGTSESATPESSGSEVEFSHEYWMRH |
| | | ALTLAKRARDEREVPVGAVLVLNNRVIGEG |
| | | WNRAIGLHDPTAHAEIMALRQGGLVMQNYR |
| | | LIDATLYVTFEPCVMCAGAMIHSRIGRVVF |
| | | GVRNAKTGAAGSLMDVLHYPGNIMNFFKTE |
| | | ITLANGEIRKRPLIETNGETGEIVWDKGRD |
| | | FATVRKVLSMPQVNIVKKTEVQTGGFSKES |
| | | ILPKRNSDKLIARKKDWDPKKYGGFDSPTV |
| | | AYSVLWAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDNEQKQLFVE |
| | | QHKHYLDEIIEQISEFSKRVILADANLDKV |
| | | LSAYNKHRDKPIREQAENIIHLFTLTNLGA |
| | | PAAFKYFDTTIDRKRYTSTKEVLDATLIHQ |
| | | SITGLYETRIDLSQLGGD |
| 123 Cas9 | 1351 | MDKKYSIGLAIGTNSVGWAVITDEYKVPSK |
| TadACP139 | | KFKVLGNTDRHSIKKNLIGALLFDSGETAE |
| ins | | ATRLKRTARRRYTRRKNRICYLQEIFSNEM |
| 1299 | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGSPEDNEQKQLFVE |
| | | QHKHYLDEIIEQISEFSKRVILADANLDKV |
| | | LSAYNKHRMNHRVEITEGILADECAALLCY |
| | | FFRMPRQVFNAQKKAQSSTDGSSGSETPGT |
| | | SESATPESSGSEVEFSHEYWMRHALTLAKR |
| | | ARDEREVPVGAVLVLNNRVIGEGWNRAIGL |
| | | HDPTAHAEIMALRQGGLVMQNYRLIDATLY |
| | | VTFEPCVMCAGAMIHSRIGRVVFGVRNAKT |
| | | GAAGSLMDVLHYPGDKPIREQAENIIHLFT |
| | | LTNLGAPAAFKYFDTTIDRKRYTSTKEVLD |
| | | ATLIHQSITGLYETRIDLSQLGGD |
| 124 Cas9 | 1352 | MDKKYSIGLAIGTNSVGWAVITDEYKVPSK |
| delta | | KFKVLGNTDRHSIKKNLIGALLFDSGETAE |
| 792-872 | | ATRLKRTARRRYTRRKNRICYLQEIFSNEM |
| TadAins | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | D |
| | | LTLLKALVRQQLPEKYKEIFFDQSKNGYAG |
| | | YIDGGASQEEFYKFIKPILEKMDGTEELLV |
| | | KLNREDLLRKQRTFDNGSIPHQIHLGELHA |
| | | ILRRQEDFYPFLKDNREKIEKILTFRIPYY |
| | | VGPLARGNSRFAWMTRKSEETITPWNFEEV |
| | | VDKGASAQSFIERMTNFDKNLPNEKVLPKH |
| | | SLLYEYFTVYNELTKVKYVTEGMRKPAFLS |
| | | GEQKKAIVDLLFKTNRKVTVKQLKEDYFKK |
| | | IECFDSVEISGVEDRFNASLGTYHDLLKII |
| | | KDKDFLDNEENEDILEDIVLTLTLFEDREM |
| | | IEERLKTYAHLFDDKVMKQLKRRRYTGWGR |
| | | LSRKLINGIRDKQSGKTILDFLKSDGFANR |
| | | NFMQLIHDDSLTFKEDIQKAQVSGQGDSLH |
| | | EHIANLAGSPAIKKGILQTVKVVDELVKVM |
| | | GRHKPENIVIEMARENQTTQKGQKNSRERM |
| | | KRIEEGIKELGSEVEFSHEYWMRHALTLAK |
| | | RARDEREVPVGAVLVLNNRVIGEGWNRAIG |
| | | LHDPTAHAEIMALRQGGLVMQNYRLIDATL |
| | | YVTFEPCVMCAGAMIHSRIGRVVFGVRNAK |
| | | TGAAGSLMDVLHYPGMNHRVEITEGILADE |
| | | CAALLCYFFRMPRQVFNAQKKAQSSTDEEV |
| | | VKKMKNYWRQLLNAKLITQRKFDNLTKAER |
| | | GGLSELDKAGFIKRQLVETRQITKHVAQIL |
| | | DSRMNTKYDENDKLIREVKVITLKSKLVSD |
| | | FRKDFQFYKVREINNYHHAHDAYLNAVVGT |
| | | ALIKKYPKLESEFVYGDYKVYDVRKMIAKS |
| | | EQEIGKATAKYFFYSNIMNFFKTEITLANG |
| | | EIRKRPLIETNGETGEIVWDKGRDFATVRK |
| | | VLSMPQVNIVKKTEVQTGGFSKESILPKRN |
| | | SDKLIARKKDWDPKKYGGFDSPTVAYSVLV |
| | | VAKVEKGKSKKLKSVKELLGITIMERSSFE |
| | | KNPIDFLEAKGYKEVKKDLIIKLPKYSLFE |
| | | LENGRKRMLASAGELQKGNELALPSKYVNF |
| | | LYLASHYEKLKGSPEDNEQKQLFVEQHKHY |
| | | LDEIIEQISEFSKRVILADANLDKVLSAYN |
| | | KHRDKPIREQAENIIHLFTLTNLGAPAAFK |
| | | YFDTTIDRKRYTSTKEVLDATLIHQSITGL |
| | | YETRIDLSQLGGD |
| 125 Cas9 | 1353 | MDKKYSIGLAIGTNSVGWAVITDEYKVPSK |
| delta | | KFKVLGNTDRHSIKKNLIGALLFDSGETAE |
| 792-906 | | ATRLKRTARRRYTRRKNRICYLQEIFSNEM |
| TadAins | | AKVDDSFFHRLEESFLVEEDKKHERHPIFG |
| polypeptide | | NIVDEVAYHEKYPTIYHLRKKLVDSTDKAD |
| sequence | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSEVEFSHEYWMRHALTLA |
| | | KRARDEREVPVGAVLVLNNRVIGEGWNRAI |
| | | GLHDPTAHAEIMALRQGGLVMQNYRLIDAT |
| | | LYVTFEPCVMCAGAMIHSRIGRVVFGVRNA |
| | | KTGAAGSLMDVLHYPGMNHRVEITEGILAD |
| | | ECAALLCYFFRMPRQVFNAQKKAQSSTDGL |
| | | SELDKAGFIKRQLVETRQITKHVAQILDSR |
| | | MNTKYDENDKLIREVKVITLKSKLVSDFRK |
| | | DFQFYKVREINNYHHAHDAYLNAVVGTALI |
| | | KKYPKLESEFVYGDYKVYDVRKMIAKSEQE |
| | | IGKATAKYFFYSNIMNFFKTEITLANGEIR |
| | | KRPLIETNGETGEIVWDKGRDFATVRKVLS |
| | | MPQVNIVKKTEVQTGGFSKESILPKRNSDK |
| | | LIARKKDWDPKKYGGFDSPTVAYSVLWAKV |
| | | EKGKSKKLKSVKELLGITIMERSSFEKNPI |
| | | DFLEAKGYKEVKKDLIIKLPKYSLFELENG |
| | | RKRMLASAGELQKGNELALPSKYVNFLYLA |
| | | SHYEKLKGSPEDNEQKQLFVEQHKHYLDEI |
| | | IEQISEFSKRVILADANLDKVLSAYNKHRD |
| | | KPIREQAENIIHLFTLTNLGAPAAFKYFDT |
| | | TIDRKRYTSTKEVLDATLIHQSITGLYETR |
| | | IDLSQLGGD |
| 126 TadA | 1354 | MDKKYSIGLAIGTNSVGWAVITDEYKVPSK |
| CP65ins | | KFKVLGNTDRHSIKKNLIGALLFDSGETAE |
| 1003 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAFMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKTAHAEIMALRQGGLVMQ |
| | | NYRLIDATLYVTFEPCVMCAGAMIHSRIGR |
| | | VVFGVRNAKTGAAGSLMDVLHYPGMNHRVE |
| | | ITEGILADECAALLCYFFRMPRQVFNAQKK |
| | | AQSSTDGSSGSETPGTSESATPESSGSEVE |
| | | FSHEYWMRHALTLAKRARDEREVPVGAVLV |
| | | LNNRVIGEGWNRAIGLHDPLESEFVYGDYK |
| | | VYDVRKMIAKSEQEIGKATAKYFFYSNIMN |
| | | FFKTEITLANGEIRKRPLIETNGETGEIVW |
| | | DKGRDFATVRKVLSMPQVNIVKKTEVQTGG |
| | | FSKESILPKRNSDKLIARKKDWDPKKYGGF |
| | | DSPTVAYSVLWAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 127 TadA | 1355 | MDKKYSIGLAIGTNSVGWAVITDEYKVPSK |
| CP65ins | | KFKVLGNTDRHSIKKNLIGALLFDSGETAE |
| 1016 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVTAHAE |
| | | IMALRQGGLVMQNYRLIDATLYVTFEPCVM |
| | | CAGAMIHSRIGRVVFGVRNAKTGAAGSLMD |
| | | VLHYPGMNHRVEITEGILADECAALLCYFF |
| | | RMPRQVFNAQKKAQSSTDGSSGSETPGTSE |
| | | SATPESSGSEVEFSHEYWMRHALTLAKRAR |
| | | DEREVPVGAVLVLNNRVIGEGWNRAIGLHD |
| | | PYDVRKMIAKSEQEIGKATAKYFFYSNIMN |
| | | FFKTEITLANGEIRKRPLIETNGETGEIVW |
| | | DKGRDFATVRKVLSMPQVNIVKKTEVQTGG |
| | | FSKESILPKRNSDKLIARKKDWDPKKYGGF |
| | | DSPTVAYSVLWAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 128 TadA | 1356 | MDKKYSIGLAIGTNSVGWAVITDEYKVPSK |
| CP65ins | | KFKVLGNTDRHSIKKNLIGALLFDSGETAE |
| 1022 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVIMTEITKAPLSASMIKRYDEHH |
| | | QDLTLLKALVRQQLPEKYKEIFFDQSKNGY |
| | | AGYIDGGASQEEFYKFIKPILEKMDGTEEL |
| | | LVKLNREDLLRKQRTFDNGSIPHQIHLGEL |
| | | HAILRRQEDFYPFLKDNREKIEKILTFRIP |
| | | YYVGPLARGNSRFAWMTRKSEETITPWNFE |
| | | EVVDKGASAQSFIERMTNFDKNLPNEKVLP |
| | | KHSLLYEYFTVYNELTKVKYVTEGMRKPAF |
| | | LSGEQKKAIVDLLFKTNRKVTVKQLKEDYF |
| | | KKIECFDSVEISGVEDRFNASLGTYHDLLK |
| | | IIKDKDFLDNEENEDILEDIVLTLTLFEDR |
| | | EMIEERLKTYAHLFDDKVMKQLKRRRYTGW |
| | | GRLSRKLINGIRDKQSGKTILDFLKSDGFA |
| | | NRNFMQLIHDDSLTFKEDIQKAQVSGQGDS |
| | | LHEHIANLAGSPAIKKGILQTVKVVDELVK |
| | | VMGRHKPENIVIEMARENQTTQKGQKNSRE |
| | | RMKRIEEGIKELGSQILKEHPVENTQLQNE |
| | | KLYLYYLQNGRDMYVDQELDINRLSDYDVD |
| | | HIVPQSFLKDDSIDNKVLTRSDKNRGKSDN |
| | | VPSEEVVKKMKNYWRQLLNAKLITQRKFDN |
| | | LTKAERGGLSELDKAGFIKRQLVETRQITK |
| | | HVAQILDSRMNTKYDENDKLIREVKVITLK |
| | | SKLVSDFRKDFQFYKVREINNYHHAHDAYL |
| | | NAVVGTALIKKYPKLESEFVYGDYKVYDVR |
| | | KMITAHAEIMALRQGGLVMQNYRLIDATLY |
| | | VTFEPCVMCAGAMIHSRIGRWFGVRNAKTG |
| | | AAGSLMDVLHYPGMNHRVEITEGILADECA |
| | | ALLCYFFRMPRQVFNAQKKAQSSTDGSSGS |
| | | ETPGTSESATPESSGSEVEFSHEYWMRHAL |
| | | TLAKRARDEREVPVGAVLVLNNRVIGEGWN |
| | | RAIGLHDPAKSEQEIGKATAKYFFYSNIMN |
| | | FFKTEITLANGEIRKRPLIETNGETGEIVW |
| | | DKGRDFATVRKVLSMPQVNIVKKTEVQTGG |
| | | FSKESILPKRNSDKLIARKKDWDPKKYGGF |
| | | DSPTVAYSVLVVAKVEKGKSKKLKSVKELL |
| | | GITIMERSSFEKNPIDFLEAKGYKEVKKDL |
| | | IIKLPKYSLFELENGRKRMLASAGELQKGN |
| | | ELALPSKYVNFLYLASHYEKLKGSPEDNEQ |
| | | KQLFVEQHKHYLDEIIEQISEFSKRVILAD |
| | | ANLDKVLSAYNKHRDKPIREQAENIIHLFT |
| | | LTNLGAPAAFKYFDTTIDRKRYTSTKEVLD |
| | | ATLIHQSITGLYETRIDLSQLGGD |
| 129 TadA | 1357 | MDKKYSIGLAIGTNSVGWAVITDEYKVPSK |
| CP65ins | | KFKVLGNTDRHSIKKNLIGALLFDSGETAE |
| 1029 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEITAHAEIMALRQGGLVMQNYRL |
| | | IDATLYVTFEPCVMCAGAMIHSRIGRVVFG |
| | | VRNAKTGAAGSLMDVLHYPGMNHRVEITEG |
| | | ILADECAALLCYFFRMPRQVFNAQKKAQSS |
| | | TDGSSGSETPGTSESATPESSGSEVEFSHE |
| | | YWMRHALTLAKRARDEREVPVGAVLVLNNR |
| | | VIGEGWNRAIGLHDPGKATAKYFFYSNIMN |
| | | FFKTEITLANGEIRKRPLIETNGETGEIVW |
| | | DKGRDFATVRKVLSMPQVNIVKKTEVQTGG |
| | | FSKESILPKRNSDKLIARKKDWDPKKYGGF |
| | | DSPTVAYSVLWAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 130 TadA | 1358 | MDKKYSIGLAIGTNSVGWAVITDEYKVPSK |
| CP65ins | | KFKVLGNTDRHSIKKNLIGALLFDSGETAE |
| 1041 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSTAHAEIMALR |
| | | QGGLVMQNYRLIDATLYVTFEPCVMCAGAM |
| | | IHSRIGRWFGVRNAKTGAAGSLMDVLHYPG |
| | | MNHRVEITEGILADECAALLCYFFRMPRQV |
| | | FNAQKKAQSSTDGSSGSETPGTSESATPES |
| | | SGSEVEFSHEYWMRHALTLAKRARDEREVP |
| | | VGAVLVLNNRVIGEGWNRAIGLHDPNIMNF |
| | | FKTEITLANGEIRKRPLIETNGETGEIVWD |
| | | KGRDFATVRKVLSMPQVNIVKKTEVQTGGF |
| | | SKESILPKRNSDKLIARKKDWDPKKYGGFD |
| | | SPTVAYSVLVVAKVEKGKSKKLKSVKELLG |
| | | ITIMERSSFEKNPIDFLEAKGYKEVKKDLI |
| | | IKLPKYSLFELENGRKRMLASAGELQKGNE |
| | | LALPSKYVNFLYLASHYEKLKGSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| 131 TadA | 1359 | MDKKYSIGLAIGTNSVGWAVITDEYKVPSK |
| CP65ins | | KFKVLGNTDRHSIKKNLIGALLFDSGETAE |
| 1054 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWKFEE |
| | | WDKGASAQSFIERMTNFDKNLPKEKVLPKH |
| | | SLLYEYFTVYNELTKVKYVTEGMRKPAFLS |
| | | GEQKKAIVDLLFKTNRKVTVKQLKEDYFKK |
| | | IECFDSVEISGVEDRFNASLGTYHDLLKII |
| | | KDKDFLDNEENEDILEDIVLTLTLFEDREM |
| | | IEERLKTYAHLFDDKVMKQLKRRRYTGWGR |
| | | LSRKLINGIRDKQSGKTILDFLKSDGFANR |
| | | NFMQLIHDDSLTFKEDIQKAQVSGQGDSLH |
| | | EHIANLAGSPAIKKGILQTVKWDELVKVMG |
| | | RHKPENIVIEMARENQTTQKGQKNSRERMK |
| | | RIEEGIKELGSQILKEHPVENTQLQNEKLY |
| | | LYYLQNGRDMYVDQELDINRLSDYDVDHIV |
| | | PQSFLKDDSIDNKVLTRSDKNRGKSDNVPS |
| | | EEVVKKMKNYWRQLLNAKLITQRKFDNLTK |
| | | AERGGLSELDKAGFIKRQLVETRQITKHVA |
| | | QILDSRMNTKYDENDKLIREVKVITLKSKL |
| | | VSDFRKDFQFYKVREINNYHHAHDAYLNAV |
| | | VGTALIKKYPKLESEFVYGDYKVYDVRKMI |
| | | AKSEQEIGKATAKYFFYSNIMNFFKTEITL |
| | | ANTAHAEIMALRQGGLVMQNYRLIDATLYV |
| | | TFEPCVMCAGAMIHSRIGRWFGVRNAKTGA |
| | | AGSLMDVLHYPGMNHRVEITEGILADECAA |
| | | LLCYFFRMPRQVFNAQKKAQSSTDGSSGSE |
| | | TPGTSESATPESSGSEVEFSHEYWMRHALT |
| | | LAKRARDEREVPVGAVLVLNNRVIGEGWNR |
| | | AIGLHDPGEIRKRPLIETNGETGEIVWDKG |
| | | RDFATVRKVLSMPQVNIVKKTEVQTGGFSK |
| | | ESILPKRNSDKLIARKKDWDPKKYGGFDSP |
| | | TVAYSVLVVAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGD |
| 132 TadA | 1360 | MDKKYSIGLAIGTNSVGWAVITDEYKVPSK |
| CP65ins | | KFKVLGNTDRHSIKKNLIGALLFDSGETAE |
| 1246 | | ATRLKRTARRRYTRRKNRICYLQEIFSNEM |
| polypeptide | | AKVDDSFFHRLEESFLVEEDKKHERHPIFG |
| sequence | | NIVDEVAYHEKYPTIYHLRKKLVDSTDKAD |
| | | LRLIYLALAHMIKFRGHFLIEGDLNPDNSD |
| | | VDKLFIQLVQTYNQLFEENPINASGVDAKA |
| | | ILSARLSKSRRLENLIAQLPGEKKNGLFGN |
| | | LIALSLGLTPNFKSNFDLAEDAKLQLSKDT |
| | | YDDDLDNLLAQIGDQYADLFLAAKNLSDAI |
| | | LLSDILRVNTEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGTYHDLLKI |
| | | IKDKDFLDNEENEDILEDIVLTLTLFEDRE |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGDSL |
| | | HEHIANLAGSPAIKKGILQTVKVVDELVKV |
| | | MGRHKPENIVIEMARENQTTQKGQKNSRER |
| | | MKRIEEGIKELGSQILKEHPVENTQLQNEK |
| | | LYLYYLQNGRDMYVDQELDINRLSDYDVDH |
| | | IVPQSFLKDDSIDNKVLTRSDKNRGKSDNV |
| | | PSEEVVKKMKNYWRQLLNAKLITQRKFDNL |
| | | TKAERGGLSELDKAGFIKRQLVETRQITKH |
| | | VAQILDSRMNTKYDENDKLIREVKVITLKS |
| | | KLVSDFRKDFQFYKVREINNYHHAHDAYLN |
| | | AVVGTALIKKYPKLESEFVYGDYKVYDVRK |
| | | MIAKSEQEIGKATAKYFFYSNIMNFFKTEI |
| | | TLANGEIRKRPLIETNGETGEIVWDKGRDF |
| | | ATVRKVLSMPQVNIVKKTEVQTGGFSKESI |
| | | LPKRNSDKLIARKKDWDPKKYGGFDSPTVA |
| | | YSVLVVAKVEKGKSKKLKSVKELLGITIME |
| | | RSSFEKNPIDFLEAKGYKEVKKDLIIKLPK |
| | | YSLFELENGRKRMLASAGELQKGNELALPS |
| | | KYVNFLYLASHYEKLKGTAHAEIMALRQGG |
| | | LVMQNYRLIDATLYVTFEPCVMCAGAMIHS |
| | | RIGRWFGVRNAKTGAAGSLMDVLHYPGMNH |
| | | RVEITEGILADECAALLCYFFRMPRQVFNA |
| | | QKKAQSSTDGSSGSETPGTSESATPESSGS |
| | | EVEFSHEYWMRHALTLAKRARDEREVPVGA |
| | | VLVLNNRVIGEGWNRAIGLHDPSPEDNEQK |
| | | QLFVEQHKHYLDEIIEQISEFSKRVILADA |
| | | NLDKVLSAYNKHRDKPIREQAENIIHLFTL |
| | | TNLGAPAAFKYFDTTIDRKRYTSTKEVLDA |
| | | TLIHQSITGLYETRIDLSQLGGD |
| TadA | 1363 | MGSHMTNDIYFMTLAIEEAKKAAQLGEVPI |
| polypeptide | | GAIITKDDEVIARAHNLRETLQQPTAHAEH |
| sequence | | IAIERAAKVLGSWRLEGCTLYVTLEPCVMC |
| | | AGTIV |
| | | MSRIPRWYGADDPKGGCSGSLMNLLQQSNF |
| | | NHRAIVDKGVLKEACSTLLTTFFKNLRANK |
| | | KSTN |
| TadA | 1364 | MTQDELYMKEAIKEAKKAEEKGEVPIGAVL |
| polypeptide | | VINGEIIARAHNLRETEQRSIAHAEMLVID |
| sequence | | EACKALGTWRLEGATLYVTLEPCPMCAGAV |
| | | VLSRVEKWFGAFDPKGGCSGTLMNLLQEER |
| | | FNHQAEVVSGVLEEECGGMLSAFFRELRKK |
| | | KKAARKNLSE |
| TadA | 1365 | MPPAFITGVTSLSDVELDHEYWMRHALTLA |
| polypeptide | | KRAWDEREVPVGAVLVHNHRVIGEGWNRPI |
| sequence | | GRHDPTAHAEIMALRQGGLVLQNYRLLDTT |
| | | LYVTLEPCVMCAGAMVHSRIGRWFGARDAK |
| | | TGAAGSLIDVLHHPGMNHRVEIIEGVLRDE |
| | | CATLLSDFFRMRRQEIKALKKADRAEGAGP |
| | | AV |
| TadA | 1366 | MDEYWMQVAMQMAEKAEAAGEVPVGAVLVK |
| polypeptide | | DGQQIATGYNLSISQHDPTAHAEILCLRSA |
| sequence | | GKKLENYRLLDATLYITLEPCAMCAGAMVH |
| | | SRIARVVYGARDEKTGAAGTVVNLLQHPAF |
| | | NHQVEVTSGVLAEACSAQLSRFFKRRRDEK |
| | | KALKLAQRAQQGIE |
| TadA | 1367 | MDAAKVRSEFDEKMMRYALELADKAEALGE |
| polypeptide | | IPVGAVLVDDARNIIGEGWNLSIVQSDPTA |
| sequence | | HAEIIALRNGAKNIQNYRLLNSTLYVTLEP |
| | | CTMCAGAILHSRIKRLVFGASDYKTGAIGS |
| | | RFHFFDDYKMNHTLEITSGVLAEECSQKLS |
| | | TFFQKRREEKKIEKALLKSLSDK |
| TadA | 1368 | MRTDESEDQDHRMMRLALDAARAAAEAGET |
| polypeptide | | PVGAVILDPSTGEVIATAGNGPIAAHDPTA |
| sequence | | HAEIAAMRAAAAKLGNYRLTDLTLWTLEPC |
| | | AMCAGAISHARIGRVVFGADDPKGGAVVHG |
| | | PKFFAQPTCHWRPEVTGGVLADESADLLRG |
| | | FFRARRKAKI |
| TadA | 1369 | MSSLKKTPIRDDAYWMGKAIREAAKAAARD |
| polypeptide | | EVPIGAVIVRDGAVIGRGHNLREGSNDPSA |
| sequence | | HAEMIAIRQAARRSANWRLTGATLYVTLEP |
| | | CLMCMGAIILARLERVVFGCYDPKGGAAGS |
| | | LYDLSADPRLNHQVRLSPGVCQEECGTMLS |
| | | DFFRDLRRRKKAKATPALFIDERKVPPEP |
| ecTadA | 1370 | MSEVEFSHEYWMRHALTLAKRARDEREVPV |
| polypeptide | | GAVLVLNNRVIGEGWNRAIGLHDPTAHAEI |
| sequence | | MALRQGGLVMQNYRLIDATLYVTFEPCVMC |
| | | AGAMIHSRIGRVVFGVRNAKTGAAGSLMDV |
| | | LHYPGMNHRVEITEGILADECAALLCYFFR |
| | | MPRQVFNAQKKAQSSTD |
| TadA*7.10 | 8 | MSEVEFSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRVVFGVRNAKTGAAGSLMDV |
| | | LHYPGMNHRVEITEGILADECAALLCYFFR |
| | | MPRQVFNAQKKAQSSTD |
| TadA*8 | 12 | MSEVEFSHEYWMRHALTLAKRARDEREVPV |
| | | GAVLVLNNRVIGEGWNRAIGLHDPTAHAEI |
| | | MALRQGGLVMQNYRLIDATLYVTFEPCVMC |
| | | AGAMIHSRIGRWFGVRNAKTGAAGSLMDVL |
| | | HYPGMNHRVEITEGILADECAALLCTFFRM |
| | | PRQVFNAQKKAQSSTD |
| gRNA | 224 | GUUUUAGAGCUAGAAAUAGCAAGUUAAAAU |
| scaffold | | AAGGCUAGUCCGUUAUCAACUUGAAAAAGU |
| nucleotide | | GGCACCGAGUCGGUGCUUUU |
| sequence | | |
| gRNA | 225 | GUUUUUGUACUCUCAAGAUUUAAGUAACUG |
| scaffold | | UACAACGAAACUUACACAGUUACUUAAAUC |
| nucleotide | | UUGCAGAAGCUACAAAGAUAAGGCUUCAUG |
| sequence | | CCGAAAUCAACACCCUGUCAUUUUAUGGCA |
| | | GGGUG |
| S. pyogenes | 226 | GUUUUAGAGCUAGAAAUAGCAAGUUAAAAU |
| gRNA | | AAGGCUAGUCCGUUAUCAACUUGAAAAAGU |
| scaffold | | GGCACCGAGUCGGUGC |
| nucleotide | | |
| sequence | | |
| S. aureus | 227 | GUUUUAGUACUCUGUAAUGAAAAUUACAGA |
| gRNA | | AUCUACUAAAACAAGGCAAAAUGCCGUGUU |
| scaffold | | UAUCUCGUCAACUUGUUGGCGAGA |
| nucleotide | | |
| sequence | | |
| BhCas12b | 228 | GUUCUGUCUUUUGGUCAGGACAACCGUCUA |
| gRNA | | GCUAUAAGUGCUGCAGGGUGUGAGAAACUC |
| scaffold | | CUAUUGCUGGACGAUGUCUCUUACGAGGCA |
| nucleotide | | UUAGCAC |
| sequence | | |
| BvCas12b | 229 | GACCUAUAGGGUCAAUGAAUCUGUGCGUGU |
| gRNA | | GCCAUAAGUAAUUAAAAAUUACCCACCACA |
| scaffold | | GGAGCACCUGAAAACAGGUGCUUGGCAC |
| nucleotide | | |
| sequence | | |
| gRNA | 230 | GUUUUAGAGCUAGAAAUAGCAAGUUAAAAU |
| scaffold | | AAGGCUAGUCCGUUAUCAACUUGAAAAAGU |
| nucleotide | | GGGACCGAGUCGGUGCUUUU |
| sequence | | |
| gRNA | 3000 | GUUUUAGAGCUAGAAAUAGCAAGUUAAAAU |
| scaffold | | AAGGCUAGUCCGUUAUCAACUUGAAAAAGU |
| nucleotide | | GGCACCGAGUCGGUGCUUUU |
| sequence | | |
| BhCas12b | 243 | GUUCUGUCUUUUGGUCAGGACAACCGUCUA |
| gRNA | | GCUAUAAGUGCUGCAGGGUGUGAGAAACUC |
| scaffold + | | CUAUUGCUGGACGAUGUCUCUUACGAGGCA |
| guide | | UUAGCACNNNNNNNNNNNNNNNNNNNN |
| sequence | | |
| BvCas12b | 244 | GACCUAUAGGGUCAAUGAAUCUGUGCGUGU |
| gRNA | | GCCAUAAGUAAUUAAAAAUUACCCACCACA |
| scaffold + | | GGAGCACCUGAAAACAGGUGCUUGGCACNN |
| guide | | NNNNNNNNNNNNNNNNNN |
| sequence | | |
| AaCas12b | 245 | GUCUAAAGGACAGAAUUUUUCAACGGGUGU |
| gRNA | | GCCAAUGGCCACUUUCCAGGUGGCAAAGCC |
| scaffold + | | CGUUGAACUUCUCAAAAAGAACGAUCUGAG |
| guide | | AAGUGGCACNNNNNNNNNNNNNNNNNNNN |
| sequence | | |
| SpyMacCas9 | 1307 | MDKKYSIGLDIGTNSVGWAVITDDYKVPSK |
| polypeptide | | KFKVLGNTDRHSIKKNLIGALLFGSGETAE |
| sequence | | ATRLKRTARRRYTRRKNRICYLQEIFSNEM |
| | | AKVDDSFFHRLEESFLVEEDKKHERHPIFG |
| | | NIVDEVAYHEKYPTIYHLRKKLADSTDKAD |
| | | LRLIYLALAHMIKFRGHFLIEGDLNPDNSD |
| | | VDKLFIQLVQIYNQLFEENPINASRVDAKA |
| | | ILSARLSKSRRLENLIAQLPGEKRNGLFGN |
| | | LIALSLGLTPNFKSNFDLAEDAKLQLSKDT |
| | | YDDDLDNLLAQIGDQYADLFLAAKNLSDAI |
| | | LLSDILRVNSEITKAPLSASMIKRYDEHHQ |
| | | DLTLLKALVRQQLPEKYKEIFFDQSKNGYA |
| | | GYIDGGASQEEFYKFIKPILEKMDGTEELL |
| | | VKLNREDLLRKQRTFDNGSIPHQIHLGELH |
| | | AILRRQEDFYPFLKDNREKIEKILTFRIPY |
| | | YVGPLARGNSRFAWMTRKSEETITPWNFEE |
| | | VVDKGASAQSFIERMTNFDKNLPNEKVLPK |
| | | HSLLYEYFTVYNELTKVKYVTEGMRKPAFL |
| | | SGEQKKAIVDLLFKTNRKVTVKQLKEDYFK |
| | | KIECFDSVEISGVEDRFNASLGAYHDLLKI |
| | | IKDKDFLDNEENEDILEDIVLTLTLFEDRG |
| | | MIEERLKTYAHLFDDKVMKQLKRRRYTGWG |
| | | RLSRKLINGIRDKQSGKTILDFLKSDGFAN |
| | | RNFMQLIHDDSLTFKEDIQKAQVSGQGHSL |
| | | HEQIANLAGSPAIKKGILQTVKIVDELVKV |
| | | MGHKPENIVIEMARENQTTQKGQKNSRERM |
| | | KRIEEGIKELGSQILKEHPVENTQLQNEKL |
| | | YLYYLQNGRDMYVDQELDINRLSDYDVDHI |
| | | VPQSFIKDDSIDNKVLTRSDKNRGKSDNVP |
| | | SEEWKKMKNYWRQLLNAKLITQRKFDNLTK |
| | | AERGGLSELDKAGFIKRQLVETRQITKHVA |
| | | QILDSRMNTKYDENDKLIREVKVITLKSKL |
| | | VSDFRKDFQFYKVREINNYHHAHDAYLNAV |
| | | VGTALIKKYPKLESEFVYGDYKVYDVRKMI |
| | | AKSEQEIGKATAKYFFYSNIMNFFKTEITL |
| | | ANGEIRKRPLIETNGETGEIVWDKGRDFAT |
| | | VRKVLSMPQVNIVKKTEIQTVGQNGGLFDD |
| | | NPKSPLEVTPSKLVPLKKELNPKKYGGYQK |
| | | PTTAYPVLLITDTKQLIPISVMNKKQFEQN |
| | | PVKFLRDRGYQQVGKNDFIKLPKYTLVDIG |
| | | DGIKRLWASSKEIHKGNQLVVSKKSQILLY |
| | | HAHHLDSDLSNDYLQNHNQQFDVLFNEIIS |
| | | FSKKCKLGKEHIQKIENVYSNKKNSASIEE |
| | | LAESFIKLLGFTQLGATSPFNFLGVKLNQK |
| | | QYKGKKDYILPCTEGTLIRQSITGLYETRV |
| | | DLSKIGED |
| NLS | 83 | PKKKRKVEGADKRTADGSEFESPKKKRKV |
| NLS | 84 | KRTADGSEFESPKKKRKV |
| NLS | 85 | KRPAATKKAGQAKKKK |
| NLS | 86 | KKTELQTTNAENKTKKL |
| NLS | 87 | KRGINDRNFWRGENGRKTR |
| NLS | 1424 | RKSGKIAAIVVKRPRKPKKKRKV |
| NLS | 90 | MDSLLMNRRKFLYQFKNVRWAKGRRETYLC |
| Linker | 1425 | (SGGS)2 |
| pNMG-B335 | 1426 | MSEVEFSHEYWMRHALTLAKRARDEREVPV |
| ABE8.1_ | | GAVLVLNNRVIGEGWNRAIGLHDPTAHAEI |
| Y147T+C | | MALRQGGLVMQNYRLIDATLYVTFEPCVMC |
| P5_NGC | | AGAMIHSRIGRVVFGVRNAKTGAAGSLMDV |
| PAM_monomer | | LHYPGMNHRVEITEGILADECAALLCTFFR |
| polypeptide | | MPRQVFNAQKKAQSSTDSGGSSGGSSGSET |
| sequence | | PGTSESATPESSGGSSGGSEIGKATAKYFF |
| | | YSNIMNFFKTEITLANGEIRKRPLIETNGE |
| | | TGEIVWDKGRDFATVRKVLSMPQVNIVKKT |
| | | EVQTGGFSKESILPKRNSDKLIARKKDWDP |
| | | KKYGGFMQPTVAYSVLVVAKVEKGKSKKLK |
| | | SVKELLGITIMERSSFEKNPIDFLEAKGYK |
| | | EVKKDLIIKLPKYSLFELENGRKRMLASAK |
| | | FLQKGNELALPSKYVNFLYLASHYEKLKGS |
| | | PEDNEQKQLFVEQHKHYLDEIIEQISEFSK |
| | | RVILADANLDKVLSAYNKHRDKPIREQAEN |
| | | IIHLFTLTNLGAPRAFKYFDTTIARKEYRS |
| | | TKEVLDATLIHQSITGLYETRIDLSQLGGD |
| | | GGSGGSGGSGGSGGSGGSGGMDKKYSIGLA |
| | | IGTNSVGWAVITDEYKVPSKKFKVLGNTDR |
| | | HSIKKNLIGALLFDSGETAEATRLKRTARR |
| | | RYTRRKNRICYLQEIFSNEMAKVDDSFFHR |
| | | LEESFLVEEDKKHERHPIFGNIVDEVAYHE |
| | | KYPTIYHLRKKLVDSTDKADLRLIYLALAH |
| | | MIKFRGHFLIEGDLNPDNSDVDKLFIQLVQ |
| | | TYNQLFEENPINASGVDAKAILSARLSKSR |
| | | RLENLIAQLPGEKKNGLFGNLIALSLGLTP |
| | | NFKSNFDLAEDAKLQLSKDTYDDDLDNLLA |
| | | QIGDQYADLFLAAKNLSDAILLSDILRVNT |
| | | EITKAPLSASMIKRYDEHHQDLTLLKALVR |
| | | QQLPEKYKEIFFDQSKNGYAGYIDGGASQE |
| | | EFYKFIKPILEKMDGTEELLVKLNREDLLR |
| | | KQRTFDNGSIPHQIHLGELHAILRRQEDFY |
| | | PFLKDNREKIEKILTFRIPYYVGPLARGNS |
| | | RFAWMTRKSEETITPWNFEEVVDKGASAQS |
| | | FIERMTNFDKNLPNEKVLPKHSLLYEYFTV |
| | | YNELTKVKYVTEGMRKPAFLSGEQKKAIVD |
| | | LLFKTNRKVTVKQLKEDYFKKIECFDSVEI |
| | | SGVEDRFNASLGTYHDLLKIIKDKDFLDNE |
| | | ENEDILEDIVLTLTLFEDREMIEERLKTYA |
| | | HLFDDKVMKQLKRRRYTGWGRLSRKLINGI |
| | | RDKQSGKTILDFLKSDGFANRNFMQLIHDD |
| | | SLTFKEDIQKAQVSGQGDSLHEHIANLAGS |
| | | PAIKKGILQTVKVVDELVKVMGRHKPENIV |
| | | IEMARENQTTQKGQKNSRERMKRIEEGIKE |
| | | LGSQILKEHPVENTQLQNEKLYLYYLQNGR |
| | | DMYVDQELDINRLSDYDVDHIVPQSFLKDD |
| | | SIDNKVLTRSDKNRGKSDNVPSEEVVKKMK |
| | | NYWRQLLNAKLITQRKFDNLTKAERGGLSE |
| | | LDKAGFIKRQLVETRQITKHVAQILDSRMN |
| | | TKYDENDKLIREVKVITLKSKLVSDFRKDF |
| | | QFYKVREINNYHHAHDAYLNAWGTALIKKY |
| | | PKLESEFVYGDYKVYDVRKMIAKSEQEGAD |
| | | KRTADGSEFESPKKKRKV |
| pNMG- | 1427 | MSEVEFSHEYWMRHALTLAKRAWDEREVPV |
| 357_ | | GAVLVHNNRVIGEGWNRPIGRHDPTAHAEI |
| ABE8.14 | | MALRQGGLVMQNYRLIDATLYVTLEPCVMC |
| with | | AGAMIHSRIGRVVFGARDAKTGAAGSLMDV |
| NGC PAM | | LHHPGMNHRVEITEGILADECAALLSDFFR |
| CP5 | | MRRQEIKAQKKAQSSTDGGSSGGSSGSETP |
| polypeptide | | GTSESATPESSGGSSGGSMSEVEFSHEYWM |
| sequence | | RHALTLAKRARDEREVPVGAVLVLNNRVIG |
| | | EGWNRAIGLHDPTAHAEIMALRQGGLVMQN |
| | | YRLIDATLYVTFEPCVMCAGAMIHSRIGRW |
| | | FGVRNAKTGAAGSLMDVLHYPGMNHRVEIT |
| | | EGILADECAALLCTFFRMPRQVFNAQKKAQ |
| | | SSTDSGGSSGGSSGSETPGTSESATPESSG |
| | | GSSGGSEIGKATAKYFFYSNIMNFFKTEIT |
| | | LANGEIRKRPLIETNGETGEIVWDKGRDFA |
| | | TVRKVLSMPQVNIVKKTEVQTGGFSKESIL |
| | | PKRNSDKLIARKKDWDPKKYGGFMQPTVAY |
| | | SVLWAKVEKGKSKKLKSVKELLGITIMERS |
| | | SFEKNPIDFLEAKGYKEVKKDLIIKLPKYS |
| | | LFELENGRKRMLASAKFLQKGNELALPSKY |
| | | VNFLYLASHYEKLKGSPEDNEQKQLFVEQH |
| | | KHYLDEIIEQISEFSKRVILADANLDKVLS |
| | | AYNKHRDKPIREQAENIIHLFTLTNLGAPR |
| | | AFKYFDTTIARKEYRSTKEVLDATLIHQSI |
| | | TGLYETRIDLSQLGGDGGSGGSGGSGGSGG |
| | | SGGSGGMDKKYSIGLAIGTNSVGWAVITDE |
| | | YKVPSKKFKVLGNTDRHSIKKNLIGALLFD |
| | | SGETAEATRLKRTARRRYTRRKNRICYLQE |
| | | IFSNEMAKVDDSFFHRLEESFLVEEDKKHE |
| | | RHPIFGNIVDEVAYHEKYPTIYHLRKKLVD |
| | | STDKADLRLIYLALAHMIKFRGHFLIEGDL |
| | | NPDNSDVDKLFIQLVQTYNQLFEENPINAS |
| | | GVDAKAILSARLSKSRRLENLIAQLPGEKK |
| | | NGLFGNLIALSLGLTPNFKSNFDLAEDAKL |
| | | QLSKDTYDDDLDNLLAQIGDQYADLFLAAK |
| | | NLSDAILLSDILRVNTEITKAPLSASMIKR |
| | | YDEHHQDLTLLKALVRQQLPEKYKEIFFDQ |
| | | SKNGYAGYIDGGASQEEFYKFIKPILEKMD |
| | | GTEELLVKLNREDLLRKQRTFDNGSIPHQI |
| | | HLGELHAILRRQEDFYPFLKDNREKIEKIL |
| | | TFRIPYYVGPLARGNSRFAWMTRKSEETIT |
| | | PWNFEEVVDKGASAQSFIERMTNFDKNLPN |
| | | EKVLPKHSLLYEYFTVYNELTKVKYVTEGM |
| | | RKPAFLSGEQKKAIVDLLFKTNRKVTVKQL |
| | | KEDYFKKIECFDSVEISGVEDRFNASLGTY |
| | | HDLLKIIKDKDFLDNEENEDILEDIVLTLT |
| | | LFEDREMIEERLKTYAHLFDDKVMKQLKRR |
| | | RYTGWGRLSRKLINGIRDKQSGKTILDFLK |
| | | SDGFANRNFMQLIHDDSLTFKEDIQKAQVS |
| | | GQGDSLHEHIANLAGSPAIKKGILQTVKVV |
| | | DELVKVMGRHKPENIVIEMARENQTTQKGQ |
| | | KNSRERMKRIEEGIKELGSQILKEHPVENT |
| | | QLQNEKLYLYYLQNGRDMYVDQELDINRLS |
| | | DYDVDHIVPQSFLKDDSIDNKVLTRSDKNR |
| | | GKSDNVPSEEWKKMKNYWRQLLNAKLITQR |
| | | KFDNLTKAERGGLSELDKAGFIKRQLVETR |
| | | QITKHVAQILDSRMNTKYDENDKLIREVKV |
| | | ITLKSKLVSDFRKDFQFYKVREINNYHHAH |
| | | DAYLNAVVGTALIKKYPKLESEFVYGDYKV |
| | | YDVRKMIAKSEQEGADKRTADGSEFESPKK |
| | | KRKV |
| ABE8.8-m | 1428 | MSEVEFSHEYWMRHALTLAKRARDEREVPV |
| polypeptide | | GAVLVLNNRVIGEGWNRAIGLHDPTAHAEI |
| sequence | | MALRQGGLVMQNYRLIDATLYVTFEPCVMC |
| | | AGAMIHSRIGRVVFGVRNAKTGAAGSLMDV |
| | | LHHPGMNHRVEITEGILADECAALLCRFFR |
| | | MPRRVFNAQKKAQSSTDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFDSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASAGELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KRYTSTKEVLDATLIHQSITGLYETRIDLS |
| | | QLGGDEGADKRTADGSEFESPKKKRKV |
| ABE8.8-d | 1429 | MSEVEFSHEYWMRHALTLAKRAWDEREVPV |
| polypeptide | | GAVLVHNNRVIGEGWNRPIGRHDPTAHAEI |
| sequence | | MALRQGGLVMQNYRLIDATLYVTLEPCVMC |
| | | AGAMIHSRIGRVVFGARDAKTGAAGSLMDV |
| | | LHHPGMNHRVEITEGILADECAALLSDFFR |
| | | MRRQEIKAQKKAQSSTDSGGSSGGSSGSET |
| | | PGTSESATPESSGGSSGGSSEVEFSHEYWM |
| | | RHALTLAKRARDEREVPVGAVLVLNNRVIG |
| | | EGWNRAIGLHDPTAHAEIMALRQGGLVMQN |
| | | YRLIDATLYVTFEPCVMCAGAMIHSRIGRW |
| | | FGVRNAKTGAAGSLMDVLHHPGMNHRVEIT |
| | | EGILADECAALLCRFFRMPRRVFNAQKKAQ |
| | | SSTDSGGSSGGSSGSETPGTSESATPESSG |
| | | GSSGGSDKKYSIGLAIGTNSVGWAVITDEY |
| | | KVPSKKFKVLGNTDRHSIKKNLIGALLFDS |
| | | GETAEATRLKRTARRRYTRRKNRICYLQEI |
| | | FSNEMAKVDDSFFHRLEESFLVEEDKKHER |
| | | HPIFGNIVDEVAYHEKYPTIYHLRKKLVDS |
| | | TDKADLRLIYLALAHMIKFRGHFLIEGDLN |
| | | PDNSDVDKLFIQLVQTYNQLFEENPINASG |
| | | VDAKAILSARLSKSRRLENLIAQLPGEKKN |
| | | GLFGNLIALSLGLTPNFKSNFDLAEDAKLQ |
| | | LSKDTYDDDLDNLLAQIGDQYADLFLAAKN |
| | | LSDAILLSDILRVNTEITKAPLSASMIKRY |
| | | DEHHQDLTLLKALVRQQLPEKYKEIFFDQS |
| | | KNGYAGYIDGGASQEEFYKFIKPILEKMDG |
| | | TEELLVKLNREDLLRKQRTFDNGSIPHQIH |
| | | LGELHAILRRQEDFYPFLKDNREKIEKILT |
| | | FRIPYYVGPLARGNSRFAWMTRKSEETITP |
| | | WNFEEWDKGASAQSFIERMTNFDKNLPNEK |
| | | VLPKHSLLYEYFTVYNELTKVKYVTEGMRK |
| | | PAFLSGEQKKAIVDLLFKTNRKVTVKQLKE |
| | | DYFKKIECFDSVEISGVEDRFNASLGTYHD |
| | | LLKIIKDKDFLDNEENEDILEDIVLTLTLF |
| | | EDREMIEERLKTYAHLFDDKVMKQLKRRRY |
| | | TGWGRLSRKLINGIRDKQSGKTILDFLKSD |
| | | GFANRNFMQLIHDDSLTFKEDIQKAQVSGQ |
| | | GDSLHEHIANLAGSPAIKKGILQTVKWDEL |
| | | VKVMGRHKPENIVIEMARENQTTQKGQKNS |
| | | RERMKRIEEGIKELGSQILKEHPVENTQLQ |
| | | NEKLYLYYLQNGRDMYVD |
| | | QELDINRLSDYDVDHIVPQSFLKDDSIDNK |
| | | VLTRSDKNRGKSDNVPSEEWKKMKNYWRQL |
| | | LNAKLITQRKFDNLTKAERGGLSELDKAGF |
| | | IKRQLVETRQITKHVAQILDSRMNTKYDEN |
| | | DKLIREVKVITLKSKLVSDFRKDFQFYKVR |
| | | EINNYHHAHDAYLNAWGTALIKKYPKLESE |
| | | FVYGDYKVYDVRKMIAKSEQEIGKATAKYF |
| | | FYSNIMNFFKTEITLANGEIRKRPLIETNG |
| | | ETGEIVWDKGRDFATVRKVLSMPQVNIVKK |
| | | TEVQTGGFSKESILPKRNSDKLIARKKDWD |
| | | PKKYGGFDSPTVAYSVLVVAKVEKGKSKKL |
| | | KSVKELLGITIMERSSFEKNPIDFLEAKGY |
| | | KEVKKDLIIKLPKYSLFELENGRKRMLASA |
| | | GELQKGNELALPSKYVNFLYLASHYEKLKG |
| | | SPEDNEQKQLFVEQHKHYLDEIIEQISEFS |
| | | KRVILADANLDKVLSAYNKHRDKPIREQAE |
| | | NIIHLFTLTNLGAPAAFKYFDTTIDRKRYT |
| | | STKEVLDATLIHQSITGLYETRIDLSQLGG |
| | | DEGADKRTADGSEFESPKKKRKV |
| ABE8.13-m | 1430 | MSEVEFSHEYWMRHALTLAKRARDEREVPV |
| polypeptide | | GAVLVLNNRVIGEGWNRAIGLHDPTAHAEI |
| sequence | | MALRQGGLVMQNYRLYDATLYVTFEPCVMC |
| | | AGAMIHSRIGRVVFGVRNAKTGAAGSLMDV |
| | | LHHPGMNHRVEITEGILADECAALLCRFFR |
| | | MPRRVFNAQKKAQSSTDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFDSPTVAYSVLWAKVEKGKS |
| | | KKLKSVKELLGITIMERSSFEKNPIDFLEA |
| | | KGYKEVKKDLIIKLPKYSLFELENGRKRML |
| | | ASAGELQKGNELALPSKYVNFLYLASHYEK |
| | | LKGSPEDNEQKQLFVEQHKHYLDEIIEQIS |
| | | EFSKRVILADANLDKVLSAYNKHRDKPIRE |
| | | QAENIIHLFTLTNLGAPAAFKYFDTTIDRK |
| | | RYTSTKEVLDATLIHQSITGLYETRIDLSQ |
| | | LGGDEGADKRTADGSEFESPKKKRKV |
| ABE8.13-d | 1431 | MSEVEFSHEYWMRHALTLAKRAWDEREVPV |
| polypeptide | | GAVLVHNNRVIGEGWNRPIGRHDPTAHAEI |
| sequence | | MALRQGGLVMQNYRLIDATLYVTLEPCVMC |
| | | AGAMIHSRIGRVVFGARDAKTGAAGSLMDV |
| | | LHHPGMNHRVEITEGILADECAALLSDFFR |
| | | MRRQEIKAQKKAQSSTDSGGSSGGSSGSET |
| | | PGTSESATPESSGGSSGGSSEVEFSHEYWM |
| | | RHALTLAKRARDEREVPVGAVLVLNNRVIG |
| | | EGWNRAIGLHDPTAHAEIMALRQGGLVMQN |
| | | YRLYDATLYVTFEPCVMCAGAMIHSRIGRV |
| | | VFGVRNAKTGAAGSLMDVLHHPGMNHRVEI |
| | | TEGILADECAALLCRFFRMPRRVFNAQKKA |
| | | QSSTDSGGSSGGSSGSETPGTSESATPESS |
| | | GGSSGGSDKKYSIGLAIGTNSVGWAVITDE |
| | | YKVPSKKFKVLGNTDRHSIKKNLIGALLFD |
| | | SGETAEATRLKRTARRRYTRRKNRICYLQE |
| | | IFSNEMAKVDDSFFHRLEESFLVEEDKKHE |
| | | RHPIFGNIVDEVAYHEKYPTIYHLRKKLVD |
| | | STDKADLRLIYLALAHMIKFRGHFLIEGDL |
| | | NPDNSDVDKLFIQLVQTYNQLFEENPINAS |
| | | GVDAKAILSARLSKSRRLENLIAQLPGEKK |
| | | NGLFGNLIALSLGLTPNFKSNFDLAEDAKL |
| | | QLSKDTYDDDLDNLLAQIGDQYADLFLAAK |
| | | NLSDAILLSDILRVNTEITKAPLSASMIKR |
| | | YDEHHQDLTLLKALVRQQLPEKYKEIFFDQ |
| | | SKNGYAGYIDGGASQEEFYKFIKPILEKMD |
| | | GTEELLVKLNREDLLRKQRTFDNGSIPHQI |
| | | HLGELHAILRRQEDFYPFLKDNREKIEKIL |
| | | TFRIPYYVGPLARGNSRFAWMTRKSEETIT |
| | | PWNFEEWDKGASAQSFIERMTNFDKNLPNE |
| | | KVLPKHSLLYEYFTVYNELTKVKYVTEGMR |
| | | KPAFLSGEQKKAIVDLLFKTNRKVTVKQLK |
| | | EDYFKKIECFDSVEISGVEDRFNASLGTYH |
| | | DLLKIIKDKDFLDNEENEDILEDIVLTLTL |
| | | FEDREMIEERLKTYAHLFDDKVMKQLKRRR |
| | | YTGWGRLSRKLINGIRDKQSGKTILDFLKS |
| | | DGFANRNFMQLIHDDSLTFKEDIQKAQVSG |
| | | QGDSLHEHIANLAGSPAIKKGILQTVKVVD |
| | | ELVKVMGRHKPENIVIEMARENQTTQKGQK |
| | | NSRERMKRIEEGIKELGSQILKEHPVENTQ |
| | | LQNEKLYLYYLQNGRDMYVDQELDINRLSD |
| | | YDVDHIVPQSFLKDDSIDNKVLTRSDKNRG |
| | | KSDNVPSEEWKKMKNYWRQLLNAKLITQRK |
| | | FDNLTKAERGGLSELDKAGFIKRQLVETRQ |
| | | ITKHVAQILDSRMNTKYDENDKLIREVKVI |
| | | TLKSKLVSDFRKDFQFYKVREINNYHHAHD |
| | | AYLNAWGTALIKKYPKLESEFVYGDYKVYD |
| | | VRKMIAKSEQEIGKATAKYFFYSNIMNFFK |
| | | TEITLANGEIRKRPLIETNGETGEIVWDKG |
| | | RDFATVRKVLSMPQVNIVKKTEVQTGGFSK |
| | | ESILPKRNSDKLIARKKDWDPKKYGGFDSP |
| | | TVAYSVLVVAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGDEGADKRTAD |
| | | GSEFESPKKKRKV |
| ABE8.17-m | 1432 | MSEVEFSHEYWMRHALTLAKRARDEREVPV |
| polypeptide | | GAVLVLNNRVIGEGWNRAIGLHDPTAHAEI |
| sequence | | MALRQGGLVMQNYRLIDATLYSTFEPCVMC |
| | | AGAMIHSRIGRVVFGVRNAKTGAAGSLMDV |
| | | LHYPGMNHRVEITEGILADECAALLCYFFR |
| | | MPRRVFNAQKKAQSSTDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFDSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASAGELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KRYTSTKEVLDATLIHQSITGLYETRIDLS |
| | | QLGGDEGADKRTADGSEFESPKKKRKV |
| ABE8.17-d | 1433 | MSEVEFSHEYWMRHALTLAKRAWDEREVPV |
| polypeptide | | GAVLVHNNRVIGEGWNRPIGRHDPTAHAEI |
| sequence | | MALRQGGLVMQNYRLIDATLYVTLEPCVMC |
| | | AGAMIHSRIGRVVFGARDAKTGAAGSLMDV |
| | | LHHPGMNHRVEITEGILADECAALLSDFFR |
| | | MRRQEIKAQKKAQSSTDSGGSSGGSSGSET |
| | | PGTSESATPESSGGSSGGSSEVEFSHEYWM |
| | | RHALTLAKRARDEREVPVGAVLVLNNRVIG |
| | | EGWNRAIGLHDPTAHAEIMALRQGGLVMQN |
| | | YRLIDATLYSTFEPCVMCAGAMIHSRIGRW |
| | | FGVRNAKTGAAGSLMDVLHYPGMNHRVEIT |
| | | EGILADECAALLCYFFRMPRRVFNAQKKAQ |
| | | SSTDSGGSSGGSSGSETPGTSESATPESSG |
| | | GSSGGSDKKYSIGLAIGTNSVGWAVITDEY |
| | | KVPSKKFKVLGNTDRHSIKKNLIGALLFDS |
| | | GETAEATRLKRTARRRYTRRKNRICYLQEI |
| | | FSNEMAKVDDSFFHRLEESFLVEEDKKHER |
| | | HPIFGNIVDEVAYHEKYPTIYHLRKKLVDS |
| | | TDKADLRLIYLALAHMIKFRGHFLIEGDLN |
| | | PDNSDVDKLFIQLVQTYNQLFEENPINASG |
| | | VDAKAILSARLSKSRRLENLIAQLPGEKKN |
| | | GLFGNLIALSLGLTPNFKSNFDLAEDAKLQ |
| | | LSKDTYDDDLDNLLAQIGDQYADLFLAAKN |
| | | LSDAILLSDILRVNTEITKAPLSASMIKRY |
| | | DEHHQDLTLLKALVRQQLPEKYKEIFFDQS |
| | | KNGYAGYIDGGASQEEFYKFIKPILEKMDG |
| | | TEELLVKLNREDLLRKQRTFDNGSIPHQIH |
| | | LGELHAILRRQEDFYPFLKDNREKIEKILT |
| | | FRIPYYVGPLARGNSRFAWMTRKSEETITP |
| | | WNFEEWDKGASAQSFIERMTNFDKNLPNEK |
| | | VLPKHSLLYEYFTVYNELTKVKYVTEGMRK |
| | | PAFLSGEQKKAIVDLLFKTNRKVTVKQLKE |
| | | DYFKKIECFDSVEISGVEDRFNASLGTYHD |
| | | LLKIIKDKDFLDNEENEDILEDIVLTLTLF |
| | | EDREMIEERLKTYAHLFDDKVMKQLKRRRY |
| | | TGWGRLSRKLINGIRDKQSGKTILDFLKSD |
| | | GFANRNFMQLIHDDSLTFKEDIQKAQVSGQ |
| | | GDSLHEHIANLAGSPAIKKGILQTVKVVDE |
| | | LVKVMGRHKPENIVIEMARENQTTQKGQKN |
| | | SRERMKRIEEGIKELGSQILKEHPVENTQL |
| | | QNEKLYLYYLQNGRDMYVDQELDINRLSDY |
| | | DVDHIVPQSFLKDDSIDNKVLTRSDKNRGK |
| | | SDNVPSEEWKKMKNYWRQLLNAKLITQRKF |
| | | DNLTKAERGGLSELDKAGFIKRQLVETRQI |
| | | TKHVAQILDSRMNTKYDENDKLIREVKVIT |
| | | LKSKLVSDFRKDFQFYKVREINNYHHAHDA |
| | | YLNAVVGTALIKKYPKLESEFVYGDYKVYD |
| | | VRKMIAKSEQEIGKATAKYFFYSNIMNFFK |
| | | TEITLANGEIRKRPLIETNGETGEIVWDKG |
| | | RDFATVRKVLSMPQVNIVKKTEVQTGGFSK |
| | | ESILPKRNSDKLIARKKDWDPKKYGGFDSP |
| | | TVAYSVLVVAKVEKGKSKKLKSVKELLGIT |
| | | IMERSSFEKNPIDFLEAKGYKEVKKDLIIK |
| | | LPKYSLFELENGRKRMLASAGELQKGNELA |
| | | LPSKYVNFLYLASHYEKLKGSPEDNEQKQL |
| | | FVEQHKHYLDEIIEQISEFSKRVILADANL |
| | | DKVLSAYNKHRDKPIREQAENIIHLFTLTN |
| | | LGAPAAFKYFDTTIDRKRYTSTKEVLDATL |
| | | IHQSITGLYETRIDLSQLGGDEGADKRTAD |
| | | GSEFESPKKKRKV |
| ABE8.20-m | 1434 | MSEVEFSHEYWMRHALTLAKRARDEREVPV |
| polypeptide | | GAVLVLNNRVIGEGWNRAIGLHDPTAHAEI |
| sequence | | MALRQGGLVMQNYRLYDATLYSTFEPCVMC |
| | | AGAMIHSRIGRWFGVRNAKTGAAGSLMDVL |
| | | HHPGMNHRVEITEGILADECAALLCRFFRM |
| | | PRRVFNAQKKAQSSTDSGGSSGGSSGSETP |
| | | GTSESATPESSGGSSGGSDKKYSIGLAIGT |
| | | NSVGWAVITDEYKVPSKKFKVLGNTDRHSI |
| | | KKNLIGALLFDSGETAEATRLKRTARRRYT |
| | | RRKNRICYLQEIFSNEMAKVDDSFFHRLEE |
| | | SFLVEEDKKHERHPIFGNIVDEVAYHEKYP |
| | | TIYHLRKKLVDSTDKADLRLIYLALAHMIK |
| | | FRGHFLIEGDLNPDNSDVDKLFIQLVQTYN |
| | | QLFEENPINASGVDAKAILSARLSKSRRLE |
| | | NLIAQLPGEKKNGLFGNLIALSLGLTPNFK |
| | | SNFDLAEDAKLQLSKDTYDDDLDNLLAQIG |
| | | DQYADLFLAAKNLSDAILLSDILRVNTEIT |
| | | KAPLSASMIKRYDEHHQDLTLLKALVRQQL |
| | | PEKYKEIFFDQSKNGYAGYIDGGASQEEFY |
| | | KFIKPILEKMDGTEELLVKLNREDLLRKQR |
| | | TFDNGSIPHQIHLGELHAILRRQEDFYPFL |
| | | KDNREKIEKILTFRIPYYVGPLARGNSRFA |
| | | WMTRKSEETITPWNFEEWDKGASAQSFIER |
| | | MTNFDKNLPNEKVLPKHSLLYEYFTVYNEL |
| | | TKVKYVTEGMRKPAFLSGEQKKAIVDLLFK |
| | | TNRKVTVKQLKEDYFKKIECFDSVEISGVE |
| | | DRFNASLGTYHDLLKIIKDKDFLDNEENED |
| | | ILEDIVLTLTLFEDREMIEERLKTYAHLFD |
| | | DKVMKQLKRRRYTGWGRLSRKLINGIRDKQ |
| | | SGKTILDFLKSDGFANRNFMQLIHDDSLTF |
| | | KEDIQKAQVSGQGDSLHEHIANLAGSPAIK |
| | | KGILQTVKVVDELVKVMGRHKPENIVIEMA |
| | | RENQTTQKGQKNSRERMKRIEEGIKELGSQ |
| | | ILKEHPVENTQLQNEKLYLYYLQNGRDMYV |
| | | DQELDINRLSDYDVDHIVPQSFLKDDSIDN |
| | | KVLTRSDKNRGKSDNVPSEEVVKKMKNYWR |
| | | QLLNAKLITQRKFDNLTKAERGGLSELDKA |
| | | GFIKRQLVETRQITKHVAQILDSRMNTKYD |
| | | ENDKLIREVKVITLKSKLVSDFRKDFQFYK |
| | | VREINNYHHAHDAYLNAVVGTALIKKYPKL |
| | | ESEFVYGDYKVYDVRKMIAKSEQEIGKATA |
| | | KYFFYSNIMNFFKTEITLANGEIRKRPLIE |
| | | TNGETGEIVWDKGRDFATVRKVLSMPQVNI |
| | | VKKTEVQTGGFSKESILPKRNSDKLIARKK |
| | | DWDPKKYGGFDSPTVAYSVLVVAKVEKGKS |
| | | KKLKSVKELLGITIMERSSFEKNPIDFLEA |
| | | KGYKEVKKDLIIKLPKYSLFELENGRKRML |
| | | ASAGELQKGNELALPSKYVNFLYLASHYEK |
| | | LKGSPEDNEQKQLFVEQHKHYLDEIIEQIS |
| | | EFSKRVILADANLDKVLSAYNKHRDKPIRE |
| | | QAENIIHLFTLTNLGAPAAFKYFDTTIDRK |
| | | RYTSTKEVLDATLIHQSITGLYETRIDLSQ |
| | | LGGDEGADKRTADGSEFESPKKKRKV |
| ABE8.20-d | 1435 | MSEVEFSHEYWMRHALTLAKRAWDEREVPV |
| polypeptide | | GAVLVHNNRVIGEGWNRPIGRHDPTAHAEI |
| sequence | | MALRQGGLVMQNYRLIDATLYVTLEPCVMC |
| | | AGAMIHSRIGRVVFGARDAKTGAAGSLMDV |
| | | LHHPGMNHRVEITEGILADECAALLSDFFR |
| | | MRRQEIKAQKKAQSSTDSGGSSGGSSGSET |
| | | PGTSESATPESSGGSSGGSSEVEFSHEYWM |
| | | RHALTLAKRARDEREVPVGAVLVLNNRVIG |
| | | EGWNRAIGLHDPTAHAEIMALRQGGLVMQN |
| | | YRLYDATLYSTFEPCVMCAGAMIHSRIGRV |
| | | VFGVRNAKTGAAGSLMDVLHHPGMNHRVEI |
| | | TEGILADECAALLCRFFRMPRRVFNAQKKA |
| | | QSSTDSGGSSGGSSGSETPGTSESATPESS |
| | | GGSSGGSDKKYSIGLAIGTNSVGWAVITDE |
| | | YKVPSKKFKVLGNTDRHSIKKNLIGALLFD |
| | | SGETAEATRLKRTARRRYTRRKNRICYLQE |
| | | IFSNEMAKVDDSFFHRLEESFLVEEDKKHE |
| | | RHPIFGNIVDEVAYHEKYPTIYHLRKKLVD |
| | | STDKADLRLIYLALAHMIKFRGHFLIEGDL |
| | | NPDNSDVDKLFIQLVQTYNQLFEENPINAS |
| | | GVDAKAILSARLSKSRRLENLIAQLPGEKK |
| | | NGLFGNLIALSLGLTPNFKSNFDLAEDAKL |
| | | QLSKDTYDDDLDNLLAQIGDQYADLFLAAK |
| | | NLSDAILLSDILRVNTEITKAPLSASMIKR |
| | | YDEHHQDLTLLKALVRQQLPEKYKEIFFDQ |
| | | SKNGYAGYIDGGASQEEFYKFIKPILEKMD |
| | | GTEELLVKLNREDLLRKQRTFDNGSIPHQI |
| | | HLGELHAILRRQEDFYPFLKDNREKIEKIL |
| | | TFRIPYYVGPLARGNSRFAWMTRKSEETIT |
| | | PWNFEEWDKGASAQSFIERMTNFDKNLPNE |
| | | KVLPKHSLLYEYFTVYNELTKVKYVTEGMR |
| | | KPAFLSGEQKKAIVDLLFKTNRKVTVKQLK |
| | | EDYFKKIECFDSVEISGVEDRFNASLGTYH |
| | | DLLKIIKDKDFLDNEENEDILEDIVLTLTL |
| | | FEDREMIEERLKTYAHLFDDKVMKQLKRRR |
| | | YTGWGRLSRKLINGIRDKQSGKTILDFLKS |
| | | DGFANRNFMQLIHDDSLTFKEDIQKAQVSG |
| | | QGDSLHEHIANLAGSPAIKKGILQTVKWDE |
| | | LVKVMGRHKPENIVIEMARENQTTQKGQKN |
| | | SRERMKRIEEGIKELGSQILKEHPVENTQL |
| | | QNEKLYLYYLQNGRDMYVDQELDINRLSDY |
| | | DVDHIVPQSFLKDDSIDNKVLTRSDKNRGK |
| | | SDNVPSEEWKKMKNYWRQLLNAKLITQRKF |
| | | DNLTKAERGGLSELDKAGFIKRQLVETRQI |
| | | TKHVAQILDSRMNTKYDENDKLIREVKVIT |
| | | LKSKLVSDFRKDFQFYKVREINNYHHAHDA |
| | | YLNAWGTALIKKYPKLESEFVYGDYKVYDV |
| | | RKMIAKSEQEIGKATAKYFFYSNIMNFFKT |
| | | EITLANGEIRKRPLIETNGETGEIVWDKGR |
| | | DFATVRKVLSMPQVNIVKKTEVQTGGFSKE |
| | | SILPKRNSDKLIARKKDWDPKKYGGFDSPT |
| | | VAYSVLVVAKVEKGKSKKLKSVKELLGITI |
| | | MERSSFEKNPIDFLEAKGYKEVKKDLIIKL |
| | | PKYSLFELENGRKRMLASAGELQKGNELAL |
| | | PSKYVNFLYLASHYEKLKGSPEDNEQKQLF |
| | | VEQHKHYLDEIIEQISEFSKRVILADANLD |
| | | KVLSAYNKHRDKPIREQAENIIHLFTLTNL |
| | | GAPAAFKYFDTTIDRKRYTSTKEVLDATLI |
| | | HQSITGLYETRIDLSQLGGDEGADKRTADG |
| | | SEFESPKKKRKV |
| 01. | 1436 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + | | AGAMIHSRIGRVVFGVRNAKTGAAGSLMDV |
| Y147T | | LHYPGMNHRVEITEGILADECAALLCTFFR |
| polypeptide | | MPRQVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 02. | 1437 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + | | AGAMIHSRIGRVVFGVRNAKTGAAGSLMDV |
| Y147R | | LHYPGMNHRVEITEGILADECAALLCRFFR |
| polypeptide | | MPRQVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 03. | 1438 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + Q154S | | AGAMIHSRIGRVVFGVRNAKTGAAGSLMDV |
| polypeptide | | LHYPGMNHRVEITEGILADECAALLCYFFR |
| sequence | | MPRSVFNAQKKAQSSTDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKWDELVKVMGRHKPENIVIEMA |
| | | RENQTTQKGQKNSRERMKRIEEGIKELGSQ |
| | | ILKEHPVENTQLQNEKLYLYYLQNGRDMYV |
| | | DQELDINRLSDYDVDHIVPQSFLKDDSIDN |
| | | KVLTRSDKNRGKSDNVPSEEWKKMKNYWRQ |
| | | LLNAKLITQRKFDNLTKAERGGLSELDKAG |
| | | FIKRQLVETRQITKHVAQILDSRMNTKYDE |
| | | NDKLIREVKVITLKSKLVSDFRKDFQFYKV |
| | | REINNYHHAHDAYLNAVVGTALIKKYPKLE |
| | | SEFVYGDYKVYDVRKMIAKSEQEIGKATAK |
| | | YFFYSNIMNFFKTEITLANGEIRKRPLIET |
| | | NGETGEIVWDKGRDFATVRKVLSMPQVNIV |
| | | KKTEVQTGGFSKESILPKRNSDKLIARKKD |
| | | WDPKKYGGFVSPTVAYSVLWAKVEKGKSKK |
| | | LKSVKELLGITIMERSSFEKNPIDFLEAKG |
| | | YKEVKKDLIIKLPKYSLFELENGRKRMLAS |
| | | ARELQKGNELALPSKYVNFLYLASHYEKLK |
| | | GSPEDNEQKQLFVEQHKHYLDEIIEQISEF |
| | | SKRVILADANLDKVLSAYNKHRDKPIREQA |
| | | ENIIHLFTLTNLGAPAAFKYFDTTIDRKQY |
| | | RSTKEVLDATLIHQSITGLYETRIDLSQLG |
| | | GDEGADKRTADGSEFESPKKKRKV |
| 04. | 1439 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + | | AGAMIHSRIGRVVFGVRNAKTGAAGSLMDV |
| Y123H | | LHHPGMNHRVEITEGILADECAALLCYFFR |
| polypeptide | | MPRQVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 05. | 1440 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYSTFEPCVMC |
| LS + | | AGAMIHSRIGRVVFGVRNAKTGAAGSLMDV |
| V82S | | LHYPGMNHRVEITEGILADECAALLCYFFR |
| polypeptide | | MPRQVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 06. | 1441 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + T166R | | AGAMIHSRIGRVVFGVRNAKTGAAGSLMDV |
| polypeptide | | LHYPGMNHRVEITEGILADECAALLCYFFR |
| sequence | | MPRQVFNAQKKAQSSRDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 07. | 1442 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + Q154R | | AGAMIHSRIGRVVFGVRNAKTGAAGSLMDV |
| polypeptide | | LHYPGMNHRVEITEGILADECAALLCYFFR |
| sequence | | MPRRVFNAQKKAQSSTDSGGSSGGSSGSET |
| | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 08. | 1443 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS | | AGAMIHSRIGRVVFGVRNAKTGAAGSLMDV |
| Y147R_ | | LHHPGMNHRVEITEGILADECAALLCRFFR |
| Q154R_ | | MPRRVFNAQKKAQSSTDSGGSSGGSSGSET |
| Y123H | | PGTSESATPESSGGSSGGSDKKYSIGLAIG |
| polypeptide | | TNSVGWAVITDEYKVPSKKFKVLGNTDRHS |
| sequence | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKWDELVKVMGRHKPENIVIEMA |
| | | RENQTTQKGQKNSRERMKRIEEGIKELGSQ |
| | | ILKEHPVENTQLQNEKLYLYYLQNGRDMYV |
| | | DQELDINRLSDYDVDHIVPQSFLKDDSIDN |
| | | KVLTRSDKNRGKSDNVPSEEVVKKMKNYWR |
| | | QLLNAKLITQRKFDNLTKAERGGLSELDKA |
| | | GFIKRQLVETRQITKHVAQILDSRMNTKYD |
| | | ENDKLIREVKVITLKSKLVSDFRKDFQFYK |
| | | VREINNYHHAHDAYLNAVVGTALIKKYPKL |
| | | ESEFVYGDYKVYDVRKMIAKSEQEIGKATA |
| | | KYFFYSNIMNFFKTEITLANGEIRKRPLIE |
| | | TNGETGEIVWDKGRDFATVRKVLSMPQVNI |
| | | VKKTEVQTGGFSKESILPKRNSDKLIARKK |
| | | DWDPKKYGGFVSPTVAYSVLVVAKVEKGKS |
| | | KKLKSVKELLGITIMERSSFEKNPIDFLEA |
| | | KGYKEVKKDLIIKLPKYSLFELENGRKRML |
| | | ASARELQKGNELALPSKYVNFLYLASHYEK |
| | | LKGSPEDNEQKQLFVEQHKHYLDEIIEQIS |
| | | EFSKRVILADANLDKVLSAYNKHRDKPIRE |
| | | QAENIIHLFTLTNLGAPAAFKYFDTTIDRK |
| | | QYRSTKEVLDATLIHQSITGLYETRIDLSQ |
| | | LGGDEGADKRTADGSEFESPKKKRKV |
| 09. | 1444 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLYDATLYVTFEPCVMC |
| LS | | AGAMIHSRIGRVVFGVRNAKTGAAGSLMDV |
| Y147R_ | | LHYPGMNHRVEITEGILADECAALLCRFFR |
| Q154R_ | | MPRRVFNAQKKAQSSTDSGGSSGGSSGSET |
| I76Y | | PGTSESATPESSGGSSGGSDKKYSIGLAIG |
| polypeptide | | TNSVGWAVITDEYKVPSKKFKVLGNTDRHS |
| sequence | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 10. | 1445 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + Y147R_ | | AGAMIHSRIGRVVFGVRNAKTGAAGSLMDV |
| Q154R_ | | LHYPGMNHRVEITEGILADECAALLCRFFR |
| T166R | | MPRRVFNAQKKAQSSRDSGGSSGGSSGSET |
| polypeptide | | PGTSESATPESSGGSSGGSDKKYSIGLAIG |
| sequence | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAWGTALIKKYPKL |
| | | ESEFVYGDYKVYDVRKMIAKSEQEIGKATA |
| | | KYFFYSNIMNFFKTEITLANGEIRKRPLIE |
| | | TNGETGEIVWDKGRDFATVRKVLSMPQVNI |
| | | VKKTEVQTGGFSKESILPKRNSDKLIARKK |
| | | DWDPKKYGGFVSPTVAYSVLVVAKVEKGKS |
| | | KKLKSVKELLGITIMERSSFEKNPIDFLEA |
| | | KGYKEVKKDLIIKLPKYSLFELENGRKRML |
| | | ASARELQKGNELALPSKYVNFLYLASHYEK |
| | | LKGSPEDNEQKQLFVEQHKHYLDEIIEQIS |
| | | EFSKRVILADANLDKVLSAYNKHRDKPIRE |
| | | QAENIIHLFTLTNLGAPAAFKYFDTTIDRK |
| | | QYRSTKEVLDATLIHQSITGLYETRIDLSQ |
| | | LGGDEGADKRTADGSEFESPKKKRKV |
| 11. | 1446 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + Y147T_ | | AGAMIHSRIGRVVFGVRNAKTGAAGSLMDV |
| Q154R | | LHYPGMNHRVEITEGILADECAALLCTFFR |
| polypeptide | | MPRRVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 12. | 1447 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYVTFEPCVMC |
| LS + Y147T_ | | AGAMIHSRIGRVVFGVRNAKTGAAGSLMDV |
| Q154S | | LHYPGMNHRVEITEGILADECAALLCTFFR |
| polypeptide | | MPRSVFNAQKKAQSSTDSGGSSGGSSGSET |
| sequence | | PGTSESATPESSGGSSGGSDKKYSIGLAIG |
| | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEWDKGASAQSFIE |
| | | RMTNFDKNLPNEKVLPKHSLLYEYFTVYNE |
| | | LTKVKYVTEGMRKPAFLSGEQKKAIVDLLF |
| | | KTNRKVTVKQLKEDYFKKIECFDSVEISGV |
| | | EDRFNASLGTYHDLLKIIKDKDFLDNEENE |
| | | DILEDIVLTLTLFEDREMIEERLKTYAHLF |
| | | DDKVMKQLKRRRYTGWGRLSRKLINGIRDK |
| | | QSGKTILDFLKSDGFANRNFMQLIHDDSLT |
| | | FKEDIQKAQVSGQGDSLHEHIANLAGSPAI |
| | | KKGILQTVKVVDELVKVMGRHKPENIVIEM |
| | | ARENQTTQKGQKNSRERMKRIEEGIKELGS |
| | | QILKEHPVENTQLQNEKLYLYYLQNGRDMY |
| | | VDQELDINRLSDYDVDHIVPQSFLKDDSID |
| | | NKVLTRSDKNRGKSDNVPSEEVVKKMKNYW |
| | | RQLLNAKLITQRKFDNLTKAERGGLSELDK |
| | | AGFIKRQLVETRQITKHVAQILDSRMNTKY |
| | | DENDKLIREVKVITLKSKLVSDFRKDFQFY |
| | | KVREINNYHHAHDAYLNAVVGTALIKKYPK |
| | | LESEFVYGDYKVYDVRKMIAKSEQEIGKAT |
| | | AKYFFYSNIMNFFKTEITLANGEIRKRPLI |
| | | ETNGETGEIVWDKGRDFATVRKVLSMPQVN |
| | | IVKKTEVQTGGFSKESILPKRNSDKLIARK |
| | | KDWDPKKYGGFVSPTVAYSVLVVAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| 13. | 1448 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLYDATLYVTFEPCVMC |
| LS | | AGAMIHSRIGRWFGVRNAKTGAAGSLMDVL |
| H123Y123H_ | | HHPGMNHRVEITEGILADECAALLCRFFRM |
| Y147R_ | | PRRVFNAQKKAQSSTDSGGSSGGSSGSETP |
| Q154R_ | | GTSESATPESSGGSSGGSDKKYSIGLAIGT |
| I76Y | | NSVGWAVITDEYKVPSKKFKVLGNTDRHSI |
| polypeptide | | KKNLIGALLFDSGETAEATRLKRTARRRYT |
| sequence | | RRKNRICYLQEIFSNEMAKVDDSFFHRLEE |
| | | SFLVEEDKKHERHPIFGNIVDEVAYHEKYP |
| | | TIYHLRKKLVDSTDKADLRLIYLALAHMIK |
| | | FRGHFLIEGDLNPDNSDVDKLFIQLVQTYN |
| | | QLFEENPINASGVDAKAILSARLSKSRRLE |
| | | NLIAQLPGEKKNGLFGNLIALSLGLTPNFK |
| | | SNFDLAEDAKLQLSKDTYDDDLDNLLAQIG |
| | | DQYADLFLAAKNLSDAILLSDILRVNTEIT |
| | | KAPLSASMIKRYDEHHQDLTLLKALVRQQL |
| | | PEKYKEIFFDQSKNGYAGYIDGGASQEEFY |
| | | KFIKPILEKMDGTEELLVKLNREDLLRKQR |
| | | TFDNGSIPHQIHLGELHAILRRQEDFYPFL |
| | | KDNREKIEKILTFRIPYYVGPLARGNSRFA |
| | | WMTRKSEETITPWNFEEWDKGASAQSFIER |
| | | MTNFDKNLPNEKVLPKHSLLYEYFTVYNEL |
| | | TKVKYVTEGMRKPAFLSGEQKKAIVDLLFK |
| | | TNRKVTVKQLKEDYFKKIECFDSVEISGVE |
| | | DRFNASLGTYHDLLKIIKDKDFLDNEENED |
| | | ILEDIVLTLTLFEDREMIEERLKTYAHLFD |
| | | DKVMKQLKRRRYTGWGRLSRKLINGIRDKQ |
| | | SGKTILDFLKSDGFANRNFMQLIHDDSLTF |
| | | KEDIQKAQVSGQGDSLHEHIANLAGSPAIK |
| | | KGILQTVKVVDELVKVMGRHKPENIVIEMA |
| | | RENQTTQKGQKNSRERMKRIEEGIKELGSQ |
| | | ILKEHPVENTQLQNEKLYLYYLQNGRDMYV |
| | | DQELDINRLSDYDVDHIVPQSFLKDDSIDN |
| | | KVLTRSDKNRGKSDNVPSEEVVKKMKNYWR |
| | | QLLNAKLITQRKFDNLTKAERGGLSELDKA |
| | | GFIKRQLVETRQITKHVAQILDSRMNTKYD |
| | | ENDKLIREVKVITLKSKLVSDFRKDFQFYK |
| | | VREINNYHHAHDAYLNAWGTALIKKYPKLE |
| | | SEFVYGDYKVYDVRKMIAKSEQEIGKATAK |
| | | YFFYSNIMNFFKTEITLANGEIRKRPLIET |
| | | NGETGEIVWDKGRDFATVRKVLSMPQVNIV |
| | | KKTEVQTGGFSKESILPKRNSDKLIARKKD |
| | | WDPKKYGGFVSPTVAYSVLVVAKVEKGKSK |
| | | KLKSVKELLGITIMERSSFEKNPIDFLEAK |
| | | GYKEVKKDLIIKLPKYSLFELENGRKRMLA |
| | | SARELQKGNELALPSKYVNFLYLASHYEKL |
| | | KGSPEDNEQKQLFVEQHKHYLDEIIEQISE |
| | | FSKRVILADANLDKVLSAYNKHRDKPIREQ |
| | | AENIIHLFTLTNLGAPAAFKYFDTTIDRKQ |
| | | YRSTKEVLDATLIHQSITGLYETRIDLSQL |
| | | GGDEGADKRTADGSEFESPKKKRKV |
| 14. | 1449 | MSEVEFSHEYWMRHALTLAKRARDEREVPV |
| monoABE8.1_ | | GAVLVLNNRVIGEGWNRAIGLHDPTAHAEI |
| bpN | | MALRQGGLVMQNYRLIDATLYSTFEPCVMC |
| LS + | | AGAMIHSRIGRVVFGVRNAKTGAAGSLMDV |
| V82S + | | LHYPGMNHRVEITEGILADECAALLCYFFR |
| Q154R | | MPRRVFNAQKKAQSSTDSGGSSGGSSGSET |
| polypeptide | | PGTSESATPESSGGSSGGSDKKYSIGLAIG |
| sequence | | TNSVGWAVITDEYKVPSKKFKVLGNTDRHS |
| | | IKKNLIGALLFDSGETAEATRLKRTARRRY |
| | | TRRKNRICYLQEIFSNEMAKVDDSFFHRLE |
| | | ESFLVEEDKKHERHPIFGNIVDEVAYHEKY |
| | | PTIYHLRKKLVDSTDKADLRLIYLALAHMI |
| | | KFRGHFLIEGDLNPDNSDVDKLFIQLVQTY |
| | | NQLFEENPINASGVDAKAILSARLSKSRRL |
| | | ENLIAQLPGEKKNGLFGNLIALSLGLTPNF |
| | | KSNFDLAEDAKLQLSKDTYDDDLDNLLAQI |
| | | GDQYADLFLAAKNLSDAILLSDILRVNTEI |
| | | TKAPLSASMIKRYDEHHQDLTLLKALVRQQ |
| | | LPEKYKEIFFDQSKNGYAGYIDGGASQEEF |
| | | YKFIKPILEKMDGTEELLVKLNREDLLRKQ |
| | | RTFDNGSIPHQIHLGELHAILRRQEDFYPF |
| | | LKDNREKIEKILTFRIPYYVGPLARGNSRF |
| | | AWMTRKSEETITPWNFEEVVDKGASAQSFI |
| | | ERMTNFDKNLPNEKVLPKHSLLYEYFTVYN |
| | | ELTKVKYVTEGMRKPAFLSGEQKKAIVDLL |
| | | FKTNRKVTVKQLKEDYFKKIECFDSVEISG |
| | | VEDRFNASLGTYHDLLKIIKDKDFLDNEEN |
| | | EDILEDIVLTLTLFEDREMIEERLKTYAHL |
| | | FDDKVMKQLKRRRYTGWGRLSRKLINGIRD |
| | | KQSGKTILDFLKSDGFANRNFMQLIHDDSL |
| | | TFKEDIQKAQVSGQGDSLHEHIANLAGSPA |
| | | IKKGILQTVKVVDELVKVMGRHKPENIVIE |
| | | MARENQTTQKGQKNSRERMKRIEEGIKELG |
| | | SQILKEHPVENTQLQNEKLYLYYLQNGRDM |
| | | YVDQELDINRLSDYDVDHIVPQSFLKDDSI |
| | | DNKVLTRSDKNRGKSDNVPSEEVVKKMKNY |
| | | WRQLLNAKLITQRKFDNLTKAERGGLSELD |
| | | KAGFIKRQLVETRQITKHVAQILDSRMNTK |
| | | YDENDKLIREVKVITLKSKLVSDFRKDFQF |
| | | YKVREINNYHHAHDAYLNAVVGTALIKKYP |
| | | KLESEFVYGDYKVYDVRKMIAKSEQEIGKA |
| | | TAKYFFYSNIMNFFKTEITLANGEIRKRPL |
| | | IETNGETGEIVWDKGRDFATVRKVLSMPQV |
| | | NIVKKTEVQTGGFSKESILPKRNSDKLIAR |
| | | KKDWDPKKYGGFVSPTVAYSVLWAKVEKGK |
| | | SKKLKSVKELLGITIMERSSFEKNPIDFLE |
| | | AKGYKEVKKDLIIKLPKYSLFELENGRKRM |
| | | LASARELQKGNELALPSKYVNFLYLASHYE |
| | | KLKGSPEDNEQKQLFVEQHKHYLDEIIEQI |
| | | SEFSKRVILADANLDKVLSAYNKHRDKPIR |
| | | EQAENIIHLFTLTNLGAPAAFKYFDTTIDR |
| | | KQYRSTKEVLDATLIHQSITGLYETRIDLS |
| | | QLGGDEGADKRTADGSEFESPKKKRKV |
| Linker | 65 | PAPAP |
| Linker | 66 | PAPAPA |
| Linker | 67 | PAPAPAP |
| Linker | 68 | PAPAPAPA |
| Linker | 69 | P(AP)4 |
| Linker | 70 | P(AP)7 |
| Linker | 71 | P(AP)10 |
| N gene | 4001 | atggatgccgacaagattgtattcaaagtc |
| (nucleic | | aataatcaggtggtctctttgaagcctgag |
| acid) | | attatcgtggatcaatatgagtacaagtac |
| | | cctgccatcaaagatttgaaaaagccctgt |
| | | ataaccctaggaaaggctcccgatttaaat |
| | | aaagcatacaagtcagttttgtcaggcatg |
| | | agcgccgccaaacttaatcctgacgatgta |
| | | tgttcctatttggcagcggcaatgcagttt |
| | | tttgaggggacatgtccggaagactggacc |
| | | agctatggaattgtgattgcacgaaaagga |
| | | gataagatcaccccaggttctctggtggag |
| | | ataaaacgtactgatgtagaagggaattgg |
| | | gctctgacaggaggcatggaactgacaaga |
| | | gaccccactgtccctgagcatgcgtcctta |
| | | gtcggtcttctcttgagtctgtataggttg |
| | | agcaaaatatccgggcaaaacactggtaac |
| | | tataagacaaacattgcagacaggatagag |
| | | cagatttttgagacagccccttttgttaaa |
| | | atcgtggaacaccatactctaatgacaact |
| | | cacaaaatgtgtgctaattggagtactata |
| | | ccaaacttcagatttttggccggaacctat |
| | | gacatgtttttctcccggattgagcatcta |
| | | taltcagcaatcagagtgggcacagttgtc |
| | | actgcttatgaagactgttcaggactggta |
| | | tcatttactgggttcataaaacaaatcaat |
| | | ctcaccgctagagaggcaatactatatttc |
| | | ttccacaagaactttgaggaagagataaga |
| | | agaatgttlgagccagggcaggagacagct |
| | | gttcctcactcttatttcatccacttccgt |
| | | tcactaggcttgagtgggaaatctccttat |
| | | tcatcaaatgctgttggtcacgtgttcaat |
| | | ctcattcactttgtaggatgctatatgggt |
| | | caagtcagatccctaaatgcaacggttatt |
| | | gctgcatgtgctcctcatgaaatgtctgtt |
| | | ctagggggctatctgggagaggaattcttc |
| | | gggaaagggacatttgaaagaagattcttc |
| | | agagatgagaaagaacttcaagaatacgag |
| | | gcggctgaactgacaaagactgacgtagca |
| | | ctggcagatgatggaactgtcaactctgac |
| | | gacgaggactacttttcaggtgaaaccaga |
| | | agtccggaggctgtttatactcgaatcatg |
| | | atgaatggaggtcgactaaagagatctcac |
| | | atacggagatatgtctcagtcagttccaat |
| | | catcaagcccgtccaaactcattcgccgag |
| | | tttctaaacaagacatattcgagtgactca |
| N gene | 4002 | MDADKIVFKVNNQVVSLKPEIIVDQYEYKY |
| (amino | | PAIKDLKKPCITLGKAPDLNKAYKSVLSGM |
| acid) | | SAAKLNPDDVCSYLAAAMQFFEGTCPEDWT |
| | | SYGIVIARKGDKITPGSLVEIKRTDVEGNW |
| | | ALTGGMELTRDPTVPEHASLVGLLLSLYRL |
| | | SKISGQNTGNYKTNIADRIEQIFETAPFVK |
| | | IVEHHTLMTTHKMCANWSTIPNFRFLAGTY |
| | | DMFFSRIEHLYSAIRVGTWTAYEDCSGLVS |
| | | FTGFIKQINLTAREAILYFFHKNFEEEIRR |
| | | MFEPGQETAVPHSYFIHFRSLGLSGKSPYS |
| | | SNAVGHVFNLIHFVGCYMGQVRSLNATVIA |
| | | ACAPHEMSVLGGYLGEEFFGKGTFERRFFR |
| | | DEKELQEYEAAELTKTDVALADDGTVNSDD |
| | | EDYFSGETRSPEAVYTRIMMNGGRLKRSHI |
| | | RRYVSVSSNHQARPNSFAEFLNKTYSSDS |
| L gene | 4003 | ctcgatcctggagaggtctatgatgaccct |
| (nucleic | | attgacccaatcgagttagaggctgaaccc |
| acid) | | agaggaacccccattgtccccaacatcttg |
| | | aggaactctgactacaatctcaactctcct |
| | | ttgatagaagatcctgctagactaatgtta |
| | | gaatggttaaaaacagggaatagaccttat |
| | | cggatgactctaacagacaattgctccagg |
| | | tctttcagagttttgaaagattatttcaag |
| | | aaggtagatttgggttctctcaaggtgggc |
| | | ggaatggctgcacagtcaatgatttctctc |
| | | tggttatatggtgcccactctgaatccaac |
| | | aggagccggagatgtataacagacttggcc |
| | | catttctattccaagtcgtcccccatagag |
| | | aagctgttgaatctcacgctaggaaataga |
| | | gggctgagaatccccccagagggagtgtta |
| | | agttgccttgagagggttgattatgataat |
| | | gcatttggaaggtatcttgccaacacgtat |
| | | tcctcttacttgttcttccatgtaatcacc |
| | | ttatacatgaacgccctagactgggatgaa |
| | | gaaaagaccatcctagcattatggaaagat |
| | | ttaacctcagtggacatcgggaaggacttg |
| | | gtaaagttcaaagaccaaatatggggactg |
| | | ctgatcgtgacaaaggactttgtttactcc |
| | | caaagttccaattgtctttttgacagaaac |
| | | tacacacttatgctaaaagatcttttcttg |
| | | tctcgcttcaactccttaatggtcttgctc |
| | | tctcccccagagccccgatactcagatgac |
| | | ttgatatctcaactatgccagctgtacatt |
| | | gctggggatcaagtcttgtctatgtgtgga |
| | | aactccggctatgaagtcatcaaaatattg |
| | | gagccatatgtcgtgaatagtttagtccag |
| | | agagcagaaaagtttaggcctctcattcat |
| | | tccttgggagactttcctgtatttataaaa |
| | | gacaaggtaagtcaacttgaagagacgttc |
| | | ggtccctgtgcaagaaggttctttagggct |
| | | ctggatcaattcgacaacatacatgacttg |
| | | gtttttgtgtttggctgltacaggcattgg |
| | | gggcacccatatatagattatcgaaagggt |
| | | ctgtcaaaactatatgatcaggttcacctt |
| | | aaaaaaatgatagataagtcctaccaggag |
| | | tgcttagcaagcgacctagccaggaggatc |
| | | cttagatggggttttgataagtactccaag |
| | | tggtatctggattcaagattcctagcccga |
| | | gaccaccccttgactccttatatcaaaacc |
| | | caaacatggccacccaaacatattgtagac |
| | | ttggtgggggatacatggcacaagctcccg |
| | | atcacgcagatctttgagattcctgaatca |
| | | atggatccgtcagaaatattggatgacaaa |
| | | tcacattctttcaccagaacgagactagct |
| | | tcttggctgtcagaaaaccgaggggggcct |
| | | gttcctagcgaaaaagttattatcacggcc |
| | | ctgtctaagccgcctgtcaatccccgagag |
| | | tttctgaggtctatagacctcggaggattg |
| | | ccagatgaagacttgataattggcctcaag |
| | | ccaaaggaacgggaattgaagattgaaggt |
| | | cgattctttgctctaatgtcatggaatcta |
| | | agattgtattttgtcatcactgaaaaactc |
| | | ttggccaactacatcttgccactttttgac |
| | | gcgctgactatgacagacaacctgaacaag |
| | | gtgtttaaaaagctgatcgacagggtcacc |
| | | gggcaagggcttttggactattcaagggtc |
| | | acatatgcatttcacctggactatgaaaag |
| | | tggaacaaccatcaaagattagagtcaaca |
| | | gaggatgtattttctgtcctagatcaagtg |
| | | tttggattgaagagagtgttttctagaaca |
| | | cacgagttttttcaaaaggcctggatctat |
| | | tattcagacagatcagacctcatcgggtta |
| | | cgggaggatcaaatatactgcttagatgcg |
| | | tccaacggcccaacctgttggaatggccag |
| | | gatggcgggctagaaggcttacggcagaag |
| | | ggctggagtctagtcagcttattgatgata |
| | | gatagagaatctcaaatcaggaacacaaga |
| | | accaaaatactagctcaaggagacaaccag |
| | | gttttatgtccgacatacatgttgtcgcca |
| | | gggctatctcaagaggggctcctctatgaa |
| | | ttggagagaatatcaaggaatgcactttcg |
| | | atatacagagccgtcgaggaaggggcatct |
| | | aagctagggctgatcatcaagaaagaagag |
| | | accatgtgtagttatgacttcctcatctat |
| | | ggaaaaacccctttgtttagaggtaacata |
| | | ttggtgcctgagtccaaaagatgggccaga |
| | | gtctcttgcgtctctaatgaccaaatagtc |
| | | aacctcgccaatataatgtcgacagtgtcc |
| | | accaatgcgctaacagtggcacaacactct |
| | | caatctttgatcaaaccgatgagggatttt |
| | | ctgctcatgtcagtacaggcagtctttcac |
| | | tacctgctatttagcccaatcttaaaggga |
| | | agagtttacaagattctgagcgctgaaggg |
| | | gagagctttctcctagccatglcaaggata |
| | | atctatctagatccttctttgggagggata |
| | | tctggaatgtccctcggaagattccatata |
| | | cgacagttctcagaccctgtctctgaaggg |
| | | ttatccttctggagagagatctggttaagc |
| | | tcccaagagtcctggattcacgcgttgtgt |
| | | caagaggctggaaacccagatcttggagag |
| | | agaacactcgagagcttcactcgccttcta |
| | | gaagatccgaccaccttaaatatcagagga |
| | | ggggccagtcctaccattctactcaaggat |
| | | gcaatcagaaaggctttatatgacgaggtg |
| | | gacaaggtggaaaatlcagagtttcgagag |
| | | gcaatcctgttgtccaagacccatagagat |
| | | aattttatactcttcttaatatctgttgag |
| | | cctctgtttcctcgatttctcagtgagcta |
| | | ttcagttcgtcttttttgggaatccccgag |
| | | tcaatcattggattgatacaaaactcccga |
| | | acgataagaaggcagtttagaaagagtctc |
| | | tcaaaaactttagaagaatccttctacaac |
| | | tcagagatccacgggattagtcggatgacc |
| | | cagacacctcagagggttgggggggtgtgg |
| | | ccttgctcttcagagagggcagatctactt |
| | | agggagatctcttggggaagaaaagtggta |
| | | ggcacgacagttcctcacccttctgagatg |
| | | ttgggattacttcccaagtcctctatttct |
| | | tgcacttgtggagcaacaggaggaggcaat |
| | | cctagagtttctgtatcagtactcccgtcc |
| | | tttgatcagtcatttttttcacgaggcccc |
| | | ctaaagggatacttgggctcgtccacctct |
| | | atgtcgacccagctattccatgcatgggaa |
| | | aaagtcactaatgttcatgtggtgaagaga |
| | | gctctatcgttaaaagaatctataaactgg |
| | | ttcattactagagattccaacttggctcaa |
| | | gctctaattaggaacattatgtctctgaca |
| | | ggccctgatttccctctagaggaggcccct |
| | | gtcttcaaaaggacggggtcagccttgcat |
| | | aggttcaagtctgccagatacagcgaagga |
| | | gggtattcttctgtctgcccgaacctcctc |
| | | tctcatatttctgttagtacagacaccatg |
| | | tctgatttgacccaagacgggaagaactac |
| | | gatttcatgttccagccattgatgctttat |
| | | gcacagacatggacatcagagctggtacag |
| | | agagacacaaggctaagagactclacgttt |
| | | cattggcacctccgatgcaacaggtgtgtg |
| | | agacccattgacgacgtgaccctggagacc |
| | | tctcagatcttcgagtttccggatgtgtcg |
| | | aaaagaatatccagaatggtttctggggct |
| | | gtgcctcacttccagaggcttcccgatatc |
| | | cgtctgagaccaggagattttgaatctcta |
| | | agcggtagagaaaagtctcaccatatcgga |
| | | tcagctcaggggctcttatactcaatctta |
| | | gtggcaattcacgactcaggatacaatgat |
| | | ggaaccatcttccctgtcaacatatacggc |
| | | aaggtttcccctagagactatttgagaggg |
| | | ctcgcaaggggagtattgataggatcctcg |
| | | atttgcttcttgacaagaatgacaaatatc |
| | | aatattaatagacctcttgaattggtctca |
| | | ggggtaatctcatatattctcctgaggcta |
| | | gataaccatccctccttgtacataatgctc |
| | | agagaaccgtctcttagaggagagatattt |
| | | tctatccctcagaaaatccccgccgcttat |
| | | ccaaccactatgaaagaaggcaacagatca |
| | | atcttgtgttatctccaacatgtgctacgc |
| | | tatgagcgagagataatcacggcgtctcca |
| | | gagaatgactggctatggatcttttcagac |
| | | tttagaagtgccaaaatgacgtacctatcc |
| | | ctcattacttaccagt |
| | | ctcatcttctactccagagggttgagagaa |
| | | acctatctaagagtatgagagataacctgc |
| | | gacaattgagttctttgatgaggcaggtgc |
| | | tgggcgggcacggagaagataccttagagt |
| | | cagacgacaacattcaacgactgctaaaag |
| | | actctttacgaaggacaagatgggtggatc |
| | | aagaggtgcgccatgcagctagaaccatga |
| | | ctggagattacagccccaacaagaaggtgt |
| | | cccgtaaggtaggatgttcagaatgggtct |
| | | gctctgctcaacaggttgcagtctctacct |
| | | cagcaaacccggcccctgtctcggagcttg |
| | | acataagggccctctctaagaggttccaga |
| | | accctttgatctcgggcttgagagtggttc |
| | | agtgggcaaccggtgctcattataagctta |
| | | agcctattctagatgatctcaatgttttcc |
| | | catctctctgccttgtagttggggacgggt |
| | | caggggggatatcaagggcagtcctcaaca |
| | | tgtttccagatgccaagcttgtgttcaaca |
| | | gtcttttagaggtgaatgacctgatggctt |
| | | ccggaacacatccactgcctccttcagcaa |
| | | tcatgaggggaggaaatgatatcgtctcca |
| | | gagtgatagatcttgactcaatctgggaaa |
| | | aaccgtccgacttgagaaacttggcaacct |
| | | ggaaatacttccagtcagtccaaaagcagg |
| | | tcaacatgtcctatgacctcattatttgcg |
| | | atgcagaagttactgacattgcatctatca |
| | | accggatcaccctgttaatgtccgattttg |
| | | cattgtctatagatggaccactctatttgg |
| | | tcttcaaaacttatgggactatgctagtaa |
| | | atccaaactacaaggctattcaacacctgt |
| | | caagagcgttcccctcggtcacagggttta |
| | | tcacccaagtaacttcgtctttttcatctg |
| | | agctctacctccgattctccaaacgaggga |
| | | agtttttcagagatgctgagtacttgacct |
| | | cttccacccttcgagaaatgagccttgtgt |
| | | tattcaattgtagcagccccaagagtgaga |
| | | tgcagagagctcgttccttgaactatcagg |
| | | atcttgtgagaggatttcctgaagaaatca |
| | | tatcaaatccttacaatgagatgatcataa |
| | | ctctgattgacagtgatgtagaatcttttc |
| | | tagtccacaagatggttgatgatcttgagt |
| | | tacagaggggaactctgtctaaagtggcta |
| | | tcattatagccatcatgatagttttctcca |
| | | acagagtcttcaacgtttccaaacccctaa |
| | | ctgacccctcgttctatccaccgtctgatc |
| | | ccaaaatcctgaggcacttcaacatatgtt |
| | | gcaglactatgatgtatctatctactgctt |
| | | taggtgacgtccctagcttcgcaagacttc |
| | | acgacctgtataacagacctataacttatt |
| | | acttcagaaagcaagtcattcgagggaacg |
| | | tttatctatcttggagttggtccaacgaca |
| | | cctcagtgttcaaaagggtagcctgtaatt |
| | | ctagcctgagtctgtcatctcactggatca |
| | | ggttgatttacaagatagtgaagactacca |
| | | gactcgttggcagcatcaaggatctatcca |
| | | gagaagtggaaagacaccttcataggtaca |
| | | acaggtggatcaccctagaggatatcagat |
| | | ctagatcatccctactagactacagttgcc |
| | | tg |
| L gene | 4004 | LDPGEVYDDPIDPIELEAEPRGTPIVPNIL |
| (amino | | RNSDYNLNSPLIEDPARLMLEWLKTGNRPY |
| acid) | | RMTLTDNCSRSFRVLKDYFKKVDLGSLKVG |
| | | GMAAQSMISLWLYGAHSESNRSRRCITDLA |
| | | HFYSKSSPIEKLLNLTLGNRGLRIPPEGVL |
| | | SCLERVDYDNAFGRYLANTYSSYLFFHVIT |
| | | LYMNALDWDEEKTILALWKDLTSVDIGKDL |
| | | VKFKDQIWGLLIVTKDFVYSQSSNCLFDRN |
| | | YTLMLKDLFLSRFNSLMVLLSPPEPRYSDD |
| | | LISQLCQLYIAGDQVLSMCGNSGYEVIKIL |
| | | EPYVVNSLVQRAEKFRPLIHSLGDFPVFIK |
| | | DKVSQLEETFGPCARRFFRALDQFDNIHDL |
| | | VFVFGCYRHWGHPYIDYRKGLSKLYDQVHL |
| | | KKMIDKSYQECLASDLARRILRWGFDKYSK |
| | | WYLDSRFLARDHPLTPYIKTQTWPPKHIVD |
| | | LVGDTWHKLPITQIFEIPESMDPSEILDDK |
| | | SHSFTRTRLASWLSENRGGPVPSEKVIITA |
| | | LSKPPVNPREFLRSIDLGGLPDEDLIIGLK |
| | | PKERELKIEGRFFALMSWNLRLYFVITEKL |
| | | LANYILPLFDALTMTDNLNKVFKKLIDRVT |
| | | GQGLLDYSRVTYAFHLDYEKWNNHQRLEST |
| | | EDVFSVLDQVFGLKRVFSRTHEFFQKAWIY |
| | | YSDRSDLIGLREDQIYCLDASNGPTCWNGQ |
| | | DGGLEGLRQKGWSLVSLLMIDRESQIRNTR |
| | | TKILAQGDNQVLCPTYMLSPGLSQEGLLYE |
| | | LERISRNALSIYRAVEEGASKLGLIIKKEE |
| | | TMCSYDFLIYGKTPLFRGNILVPESKRWAR |
| | | VSCVSNDQIVNLANIMSTVSTNALTVAQHS |
| | | QSLIKPMRDFLLMSVQAVFHYLLFSPILKG |
| | | RVYKILSAEGESFLLAMSRIIYLDPSLGGI |
| | | SGMSLGRFHIRQFSDPVSEGLSFWREIWLS |
| | | SQESWIHALCQEAGNPDLGERTLESFTRLL |
| | | EDPTTLNIRGGASPTILLKDAIRKALYDEV |
| | | DKVENSEFREAILLSKTHRDNFILFLISVE |
| | | PLFPRFLSELFSSSFLGIPESIIGLIQNSR |
| | | TIRRQFRKSLSKTLEESFYNSEIHGISRMT |
| | | QTPQRVGGVWPCSSERADLLREISWGRKWG |
| | | TTVPHPSEMLGLLPKSSISCTCGATGGGNP |
| | | RVSVSVLPSFDQSFFSRGPLKGYLGSSTSM |
| | | STQLFHAWEKVTNVHVVKRALSLKESINWF |
| | | ITRDSNLAQALIRNIMSLTGPDFPLEEAPV |
| | | FKRTGSALHRFKSARYSEGGYSSVCPNLLS |
| | | HISVSTDTMSDLTQDGKNYDFMFQPLMLYA |
| | | QTWTSELVQRDTRLRDSTFHWHLRCNRCVR |
| | | PIDDVTLETSQIFEFPDVSKRISRMVSGAV |
| | | PHFQRLPDIRLRPGDFESLSGREKSHHIGS |
| | | AQGLLYSILVAIHDSGYNDGTIFPVNIYGK |
| | | VSPRDYLRGLARGVLIGSSICFLTRMTNIN |
| | | INRPLELVSGVISYILLRLDNHPSLYIMLR |
| | | EPSLRGEIFSIPQKIPAAYPTTMKEGNRSI |
| | | LC |
| | | YLQHVLRYEREIITASPENDWLWIFSDFRS |
| | | AKMTYLSLITYQSHLLLQRVERNLSKSMRD |
| | | NLRQLSSLMRQVLGGHGEDTLESDDNIQRL |
| | | LKDSLRRTRWVDQEVRHAARTMTGDYSPNK |
| | | KVSRKVGCSEWVCSAQQVAVSTSANPAPVS |
| | | ELDIRALSKRFQNPLISGLRWQWATGAHYK |
| | | LKPILDDLNVFPSLCLWGDGSGGISRAVLN |
| | | MFPDAKLVFNSLLEVNDLMASGTHPLPPSA |
| | | IMRGGNDIVSRVIDLDSIWEKPSDLRNLAT |
| | | WKYFQSVQKQVNMSYDLIICDAEVTDIASI |
| | | NRITLLMSDFALSIDGPLYLVFKTYGTMLV |
| | | NPNYKAIQHLSRAFPSVTGFITQVTSSFSS |
| | | ELYLRFSKRGKFFRDAEYLTSSTLREMSLV |
| | | LFNCSSPKSEMQRARSLNYQDLVRGFPEEI |
| | | ISNPYNEMIITLIDSDVESFLVHKMVDDLE |
| | | LQRGTLSKVAIIIAIMIVFSNRVFNVSKPL |
| | | TDPSFYPPSDPKILRHFNICCSTMMYLSTA |
| | | LGDVPSFARLHDLYNRPITYYFRKQVIRGN |
| | | VYLSWSWSNDTSVFKRVACNSSLSLSSHWI |
| | | RLIYKIVKTTRLVGSIKDLSREVERHLHRY |
| | | NRWITLEDIRSRSSLLDYSCL |
| M gene | 4005 | ttctagaagcagagaggaatctttgtcctc |
| (nucleic | | ttcggacctttgtgtctgaagagacatgtc |
| acid) | | agaccatagttgacatgctctcgggttcat |
| | | gttgatacaccagactctgccctggatatg |
| | | acactgttttgcaatcactcttatttgcaa |
| | | tccgacgaactcagtatcatcatcccaagt |
| | | gatctcctgagagtattccaactcctcccc |
| | | ltcaagagggcccctggaatcagcccactg |
| | | gaagataaaggttctcctcaatttgtatac |
| | | ccagttcaggccctcagggactggagatcc |
| | | tgacaaagccagtccaataaccactttgac |
| | | taacccgatcatcctatgattcccagaata |
| | | tatctcgtcgaatgatttcagaatgtgccg |
| | | caggatcctgaacgagtaaccattcgggct |
| | | acacactttaacccttccgttgatacaaaa |
| | | gttcctcatgttcttcttgcctgtaagttc |
| | | tttcagcgggacgtattcagggggtggaag |
| | | ccacaagtcatcgtcatccagaggggctga |
| | | cgcgggagaggatttttgagtgtcctcgtc |
| | | cctgcggtttttcactatcttacgtaggag |
| | | gtt |
| M gene | 4006 | NLLRKIVKNRRDEDTQKSSPASAPLDDDDL |
| (amino | | WLPPPEYVPLKELTGKKNMRNFCINGRVKV |
| acid) | | CSPNGYSFRILRHILKSFDEIYSGNHRMIG |
| | | LVKVVIGLALSGSPVPEGLNWVYKLRRTFI |
| | | FQWADSRGPLEGEELEYSQEITWDDDTEFV |
| | | GLQIRVIAKQCHIQGRVWCINMNPRACQLW |
| | | SDMSLQTQRSEEDKDSSLLLE |
| P gene | 4007 | agcaagalctttgtcaatcctagtgctatt |
| (nucleic | | agagccggtctggccgatcttgagatggct |
| acid) | | gaagaaactgttgatctgatcaatagaaat |
| | | atcgaagacaatcaggctcatctccaaggg |
| | | gaacccatagaggtggacaatctccctgag |
| | | gatatggggcgacttcacctggatgatgga |
| | | aaatcgcccaaccatggtgagatagccaag |
| | | gtgggagaaggcaagtatcgagaggacttt |
| | | cagatggatgaaggagaggatcctagcttc |
| | | ctgttccagtcatacctggaaaatgttgga |
| | | gtccaaatagtcagacaaatgaggtcagga |
| | | gagagatttctcaagatatggtcacagacc |
| | | gtagaagagattatatcctatgtcgcggtc |
| | | aactttcccaaccctccaggaaagtcttca |
| | | gaggataaatcaacccagactactggccga |
| | | gagctcaagaaggagacaacacccactcct |
| | | tctcagagagaaagccaatcatcgaaagcc |
| | | aggatggcggctcaaattgcttctggccct |
| | | ccagcccttgaatggtcggctaccaatgaa |
| | | gaggatgatctatcagtggaggctgagatc |
| | | gctcaccagattgcagaaagtttctccaaa |
| | | aaatataagtttccctctcgatcctcaggg |
| | | atactcttgtataattttgagcaattgaaa |
| | | atgaaccttgatgatatagttaaagaggca |
| | | aaaaatgtaccaggtgtgacccgtttagcc |
| | | catgacgggtccaaactccccctaagatgt |
| | | gtactgggatgggtcgctttggccaactct |
| | | aagaaattccagttgttagtcgaatccgac |
| | | aagctgagtaaaatcatgcaagatgacttg |
| | | aatcgctatacatcttgc |
| P gene | 4008 | SKIFVNPSAIRAGLADLEMAEETVDLINRN |
| (amino | | IEDNQAHLQGEPIEVDNLPEDMGRLHLDDG |
| acid) | | KSPNHGEIAKVGEGKYREDFQMDEGEDPSF |
| | | LFQSYLENVGVQIVRQMRSGERFLKIWSQT |
| | | VEEIISYVAVNFPNPPGKSSEDKSTQTTGR |
| | | ELKKETTPTPSQRESQSSKARMAAQIASGP |
| | | PALEWSATNEEDDLSVEAEIAHQIAESFSK |
| | | KYKFPSRSSGILLYNFEQLKMNLDDIVKEA |
| | | KNVPGVTRLAHDGSKLPLRCVLGWVALANS |
| | | KKFQLLVESDKLSKIMQDDLNRYTSC |
| G gene | 4009 | atggttcctcaggctctcctgtttgtaccc |
| (nucleic | | cttctggtttttccattgtgttttgggaaa |
| acid) | | ttccctatttacacgataccagacaagctt |
| | | ggtccctggagtccgattgacatacatcac |
| | | ctcagctgcccaaacaatttggtagtggag |
| | | gacgaaggatgcaccaacctgtcagggttc |
| | | tcctacatggaacttaaagttggatacatc |
| | | ttagccataaaagtgaacgggttcacttgc |
| | | acaggcgttgtgacggaggctgaaacctac |
| | | actaacttcgttggttatgtcacaaccacg |
| | | ttcaaaagaaagcatttccgcccaacacca |
| | | gatgcatgtagagccgcgtacaactggaag |
| | | atggccggtgaccccagatatgaagagtct |
| | | ctacacaatccgtaccctgactaccgctgg |
| | | cttcgaactgtaaaaaccaccaaggagtct |
| | | ctcgttatcatatctccaagtgtggcagat |
| | | ttggacccatatgacagatcccttcactcg |
| | | agggtcttccctagcgggaagtgctcagga |
| | | gtagcggtgtcttctacctactgctccact |
| | | aaccacgattacacca |
| | | tttggatgcccgagaatccgagactaggga |
| | | tgtcttgtgacatttttaccaatagtagag |
| | | ggaagagagcatccaaagggagtgagactt |
| | | gcggctttgtagatgaaagaggcctatata |
| | | agtctttaaaaggagcatgcaaactcaagt |
| | | tatgtggagttctaggacttagacttatgg |
| | | atggaacatgggtctcgatgcaaacatcaa |
| | | atgaaaccaaatggtgccctcccgataagt |
| | | tggtgaacctgcacgactttcgctcagacg |
| | | aaattgagcaccttgttgtagaggagttgg |
| | | tcaggaagagagaggagtgtctggatgcac |
| | | tagagtccatcatgacaaccaagtcagtga |
| | | gtttcagacgtctcagtcatttaagaaaac |
| | | ttgtccctgggtttggaaaagcatatacca |
| | | tattcaacaagaccttgatggaagccgatg |
| | | ctcactacaagtcagtcagaacttggaatg |
| | | agatcctcccttcaaaagggtgtttaagag |
| | | ttggggggaggtgtcatcctcatgtgaacg |
| | | gggtgtttttcaatggtataatattaggac |
| | | ctgacggcaatgtcttaatcccagagatgc |
| | | aatcatccctcctccagcaacatatggagt |
| | | tgttggaatcctcggttatcccccttgtgc |
| | | accccctggcagacccgtctaccgttttca |
| | | aggacggtgacgaggctgaggattttgttg |
| | | aagttcaccttcccgatgtgcacaatcagg |
| | | tctcaggagttgacttgggtctcccgaact |
| | | gggggaagtatgtattactgagtgcagggg |
| | | ccctgactgccttgatgttgataattttcc |
| | | tgatgacatgttgtagaagagtcaatcgat |
| | | cagaacctacgcaacacaatctcagaggga |
| | | cagggagggaggtgtcagtcactccccaaa |
| | | gcgggaagatcatatcttcatgggaatcac |
| | | acaagagtgggggtgagaccagactg |
| G gene | 4010 | MVPQALLFVPLLVFPLCFGKFPIYTIPDKL |
| (amino | | GPWSPIDIHHLSCPNNLVVEDEGCTNLSGF |
| acid) | | SYMELKVGYILAiKVNGFTCTGVVTEAETY |
| | | TNFVGYVTTTFKRKHFRPTPDACRAAYNWK |
| | | MAGDPRYEESLHNPYPDYRWLRTVKTTKES |
| | | LVIISPSVADLDPYDRSLHSRVFPSGKCSG |
| | | VAVSSTYCSTNHDYTIWMPENPRLGMSCDI |
| | | FTNSRGKRASKGSETCGFVDERGLYKSLKG |
| | | ACKLKLCGVLGLRLMDGTWVSMQTSNETKW |
| | | CPPDKLVNLHDFRSDEIEHLVVEELVRKRE |
| | | ECLDALESIMTTKSVSFRRLSHLRKLVPGF |
| | | GKAYTIFNKTLMEADAHYKSVRTWNEILPS |
| | | KGCLRVGGRCHPHVNGVFFNGIILGPDGNV |
| | | LIPEMQSSLLQQHMELLESSVIPLVHPLAD |
| | | PSTVFKDGDEAEDFVEVHLPDVHNQVSGVD |
| | | LGLPNWGKYVLLSAGALTALMLIIFLMTCC |
| | | RRVNRSEPTQHNLRGTGREVSVTPQSGKII |
| | | SSWESHKSGGETRL |
| HEK2-2 | 4011 | gaacacaaagcatagactgc |
| target | | |
| ABCA4 | 4012 | tgtcggagttcgccctggag |
| target | | |

1. A recombinant rabies virus genome, comprising a nucleic acid encoding a therapeutic transgene, wherein:
the genome lacks a G gene encoding for a rabies virus glycoprotein or a functional variant thereof; and/or
the genome lacks an L gene encoding for a rabies virus polymerase or a functional variant thereof.

2. The recombinant genome of claim 1, wherein the genome lacks a G gene encoding for a rabies virus glycoprotein or a functional variant thereof, optionally wherein the genome further lacks an L gene encoding for a rabies virus polymerase or a functional variant thereof.

3. (canceled)

4. The recombinant genome of claim 1, wherein:
the genome comprises:
an N gene encoding for a rabies virus nucleoprotein or a functional variant thereof;
a P gene encoding for a rabies virus phosphoprotein or a functional variant thereof; and
an M gene encoding for a rabies virus matrix protein or a functional variant thereof or the genome lacks;
an N gene encoding for a rabies virus nucleoprotein or a functional variant thereof;
a P gene encoding for a rabies virus phosphoprotein or a functional variant thereof; and/or
an M gene encoding for a rabies virus matrix protein or a functional variant thereof.

5. (canceled)

6. A recombinant rabies virus particle, comprising a rabies virus glycoprotein and the recombinant rabies virus genome of claim 1.

7. A recombinant rabies virus particle, comprising:
a rabies virus glycoprotein; and
a recombinant rabies virus genome comprising a nucleic acid encoding a therapeutic transgene, wherein:
the genome lacks a G gene encoding for a rabies virus glycoprotein or a functional variant thereof; and/or
the genome lacks an L gene encoding for a rabies virus polymerase or a functional variant thereof.

8-11. (canceled)

12. The recombinant virus particle of claim 7, wherein the recombinant rabies virus is replication incompetent and/or replication deficient.

13. (canceled)

14. The recombinant genome of claim 1, wherein each of the genes are operably linked to a transcriptional regulatory element, optionally wherein:
the transcriptional regulatory element comprises a transcription initiation signal, optionally wherein the transcription initiation signal is exogenous or endogenous to the rabies virus; and/or
each of the genes are operably linked to a transcription termination polyadenylation signal.

15-18. (canceled)

19. The recombinant genome of claim 1, wherein the therapeutic transgene comprises a sequence that encodes a nucleic acid editing system or a component thereof, optionally wherein the nucleic acid editing system or component thereof comprises a Clustered Regulatory Interspaced Short Palindromic Repeat (CRISPR) system, a zinc finger nuclease (ZFN), a meganuclease, and a Transcription Activator-Like Effector-based Nucleases (TALEN).

20-21. (canceled)

22. The recombinant genome of claim 19, wherein the CRISPR-system comprises a nucleobase editor comprising a polynucleotide programmable nucleotide binding domain and a nucleobase editing domain, optionally wherein the nucleobase editing domain comprises an adenosine deaminase, cytidine deaminase, or a functional variant thereof, optionally wherein:
the adenosine deaminase comprises a TadA deaminase from any of the adenosine base editors recited in Table 10, Table 11, Table 12, Table 13, Table 14, or Table 15;
the nucleobase editing domain comprises a adenosine deaminase from any one of the adenosine base editors of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58; or
the cytidine deaminase is selected from the group consisting of: Petromyzon marinus cytosine deaminase 1 (PmCDA1), Activation-induced cytidine deaminase (AID), and APOBEC.

23-38. (canceled)

39. The recombinant genome of claim 1, wherein the nucleic acid encoding the therapeutic transgene is greater than: about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, about 2,000 bp, about 2,100 bp, about 2,200 bp, about 2,300 bp, about 2,400 bp, about 2,500 bp, about 2,600 bp, about 2,700 bp, about 2,800 bp, about 2,900 bp, or about 3,000 bp.

40-42. (canceled)

43. The recombinant genome or claim 1, wherein the nucleic acid encoding the therapeutic transgene is greater than about 3,000 bp, greater than about 4,500 bp, greater than about 8,500 bp, or greater than about 10,000 bp.

44-51. (canceled)

52. A pharmaceutical composition comprising the recombinant virus particle of claim 6.

53. A method for expressing a therapeutic transgene in a target cell, comprising transducing a target cell with the recombinant virus particle claim 6.

54. A method for expressing a nucleobase editor in a target cell, comprising transducing a target cell with a recombinant rabies virus particle, wherein the recombinant virus particle comprises:
a rabies virus glycoprotein or variant thereof; and
a recombinant rabies virus genome comprising a nucleic acid encoding a nucleobase editor comprising a polynucleotide programmable nucleotide binding domain and a nucleobase editing domain, wherein:
the genome lacks a G gene encoding for a rabies virus glycoprotein or a functional variant thereof; and/or
the genome lacks an L gene encoding for a rabies virus polymerase or a functional variant thereof.

55. The method of claim 54, wherein:
the genome comprises:
an N gene encoding for a rabies virus nucleoprotein or a functional variant thereof;
a P gene encoding for a rabies virus phosphoprotein or a functional variant thereof; and
an M gene encoding for a rabies virus matrix protein or a functional variant thereof; or
the genome lacks:
an N gene encoding for a rabies virus nucleoprotein or a functional variant thereof;
a P gene encoding for a rabies virus phosphoprotein or a functional variant thereof; and/or
an M gene encoding for a rabies virus matrix protein or a functional variant thereof.

56-61. (canceled)

62. The method of claim 54, wherein the nucleobase editing domain comprises an adenosine deaminase, cytidine deaminase, or a functional variant thereof, optionally wherein:
the adenosine deaminase comprises a TadA deaminase from any of the adenosine base editors recited in Table 10, Table 11, Table 12, Table 13, Table 14, or Table 15;
the nucleobase editing domain comprises a adenosine deaminase from any one of the adenosine base editors of: ABE 0.1, ABE 0.2, ABE 1.1, ABE 1.2, ABE2.1, ABE2.2, ABE2.3, ABE2.4, ABE2.5, ABE2.6, ABE2.7, ABE2.8, ABE2.9, ABE2.10, ABE2.11, ABE2.12, ABE3.1, ABE3.2, ABE3.3, ABE3.4, ABE3.5, ABE3.6, ABE3.7, ABE3.8, ABE4.1, ABE4.2, ABE4.3, ABE5.1, ABE5.2, ABE5.3, ABE5.4, ABE5.5, ABE5.6, ABE5.7, ABE5.8, ABE5.9, ABE5.10, ABE5.11, ABE5.12, ABE5.13, ABE5.14, ABE6.1, ABE6.2, ABE6.3, ABE6.4, ABE6.5, ABE6.6, ABE7.1, ABE7.2, ABE7.3, ABE7.4, ABE7.5, ABE7.6, ABE7.7, ABE7.8, ABE 7.9, ABE7.10, ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, ABE8.24-d, ABE8a-m, ABE8b-m, ABE8c-m, ABE8d-m, ABE8e-m, ABE8a-d, ABE8b-d, ABE8c-d, ABE8d-d, ABE8e-d, ABE9.1, ABE9.2, ABE9.3, ABE9.4, ABE9.5, ABE9.6, ABE9.7, ABE9.8, ABE9.9, ABE9.10, ABE9.11, ABE9.12, ABE9.13, ABE9.14, ABE9.15, ABE9.16, ABE9.17, ABE9.18, ABE9.19, ABE9.2, ABE9.21, ABE9.22, ABE9.23, ABE9.24, ABE9.25, ABE9.26, ABE9.27, ABE9.28, ABE9.29, ABE9.30, ABE9.31, ABE9.32, ABE9.33, ABE9.34, ABE9.35, ABE9.36, ABE9.37, ABE9.38, ABE9.39, ABE9.40, ABE9.41, ABE9.42, ABE9.43, ABE9.44, ABE9.45, ABE9.46, ABE9.47, ABE9.48, ABE9.49, ABE9.50, ABE9.51, ABE9.52, ABE9.53, ABE9.54, ABE9.55, ABE9.56, ABE9.57, and ABE9.58; or
the cytidine deaminase is selected from the group consisting of: Petromyzon marinus cytosine deaminase 1 (PmCDA1), Activation-induced cytidine deaminase (AID), and APOBEC.

63-69. (canceled)

70. The method of claim 54, wherein:
the polynucleotide programmable nucleotide binding domain comprises a Cas9 polypeptide, a Cas12 polypeptide, or a functional variant thereof; and/or
the recombinant genome further comprises a nucleic acid sequence encoding a guide RNA (gRNA) or a guide RNA (gRNA) is provided to the target cell exogenously.

71-82. (canceled)

83. A method for delivering a therapeutic transgene to a subject, comprising administering to the subject the recombinant virus particle of claim 6.

84-100. (canceled)

101. A packaging system for the recombinant preparation of a rabies virus particle, wherein the packaging system comprises:
an N gene encoding for a rabies virus nucleoprotein or a functional variant thereof;
a P gene encoding for a rabies virus phosphoprotein or a functional variant thereof;
an L gene encoding for a rabies virus polymerase or a functional variant thereof; and
a recombinant rabies virus genome, wherein:
the genome lacks a G gene encoding for a rabies virus glycoprotein or a functional variant thereof; and/or
the genome lacks an L gene encoding for a rabies virus polymerase or a functional variant thereof.

102-103. (canceled)

104. The packaging system of claim 101, wherein the N, P, and L genes are each comprised within a separate vector, optionally wherein each of the N, P, and L genes are operably linked to a transcriptional regulatory element.

105-109. (canceled)

110. The packaging system of claim 101, wherein the N, P, and L genes are comprised within a single vector, optionally wherein the single vector comprises a first expression cassette comprising the N and P genes, and a second expression cassette comprising the L gene.

111. (canceled)

112. The packaging system of claim 110, wherein:
the first expression cassette comprises from 5′ to 3′:
a transcriptional regulatory element;
the P gene; and
the N gene; or
the first expression cassette comprises from 5′ to 3′:
a transcriptional regulatory element;
the P gene;
a ribosomal skipping element; and
the N gene.

113-115. (canceled)

116. The packaging system of claim 110, wherein the second expression cassette comprises from 5′ to 3′:
a transcriptional regulatory element; and
the L gene.

117-131. (canceled)

132. A method for producing a recombinant rabies virus particle, the method comprising introducing the packaging system of claim 101 into a cell under conditions operative for enveloping the recombinant rabies virus genome to form the recombinant rabies virus particle.

133. (canceled)

134. A recombinant rabies virus particle packaging cell comprising the packaging system of claim 101.

135. A recombinant rabies virus particle packaging cell, comprising:
an N gene encoding for a rabies virus nucleoprotein or a functional variant thereof;
a P gene encoding for a rabies virus phosphoprotein or a functional variant thereof; and
an L gene encoding for a rabies virus polymerase or a functional variant thereof.

136. The packaging cell of claim 135, wherein a first expression cassette comprises the N and P genes, optionally wherein:
the first expression cassette comprises from 5′ to 3′:
a transcriptional regulatory element;
the P gene; and
the N gene; or
the first expression cassette comprises from 5′ to 3′:
a transcriptional regulatory element;
the P gene;
a ribosomal skipping element; and
the N gene.

137-152. (canceled)

153. A method for producing a recombinant rabies virus, the method comprising introducing into the packaging cell of claim 134, a nucleic acid comprising a recombinant rabies virus genome comprising a nucleic acid comprising a therapeutic transgene, under conditions operative for enclosing the recombinant rabies virus genome in the glycoprotein to form the recombinant rabies virus, wherein:
the genome lacks an endogenous G gene encoding for a rabies virus glycoprotein; and
the genome lacks an endogenous L gene encoding for a rabies virus polymerase.

154-159. (canceled)

160. The method of claim 153, wherein the recombinant rabies virus titer is greater than about 1E8 TU/mL, optionally wherein the recombinant rabies virus titer is from about 1E8 TU/mL to about 1E9 TU/mL.

161. (canceled)

162. A method of treating a disease or disorder in a subject, the method comprising administering the recombinant rabies virus particle of claim 6 to the subject, optionally wherein the disease or disorder is a neurologic disease or disorder or an ophthalmic disease or disorder.

163-165. (canceled)
